SEQUENCE LISTING (36 sequences)

SEQ ID NO: 1 - DNA, 3339 bp, synthetic construct
  misc_feature 1..3339: note = Description of sequence: Gene: HLA-A
  source 1..3339: mol_type = other DNA
agattctccccagacgccgaggatggccgtcatggcgccccgaaccctcctcctgctactctcgggggccctggccctgacccagacctgggcgggtgagtgcggggtcgggagggaaaccgcctctgcggggagaagcaaggggccctcctggcgggggcgcaggaccgggggagccgcgccgggacgagggtcgggcaggtctcagccactgctcgcccccaggctcccactccatgaggtatttcttcacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctactacaaccagagcgaggccggtgagtgaccccggccgggggcgcaggtcaggacccctcatcccccacggacgggccaggtcgcccacagtctccgggtccgagatccaccccgaagccgcgggaccccgagacccttgccccgggagaggcccaggcgcctttacccggtttcattttcagtttaggccaaaaatccccccgggttggtcggggctgggcggggctcgggggactgggctgaccgcggggtcggggccaggttctcacaccatccagataatgtatggctgcgacgtggggtcggacgggcgcttcctccgcgggtaccggcaggacgcctacgacggcaaggattacatcgccctgaacgaggacctgcgctcttggaccgcggcggacatggcggctcagatcaccaagcgcaagtgggaggcggcccatgaggcggagcagttgagagcctacctggatggcacgtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcaccctccctctggtcctgagggagaggaatcctcctgggttccagatcctgtaccagagagtgactctgaggttccgccctgctctctgacacaattaagggataaaatctctgaaggagtgacgggaagacgatccctcgaatactgatgagtggttccctttgacaccggcagcagccttgggcccgtgacttttcctctcaggccttgttctctgcttcacactcaatgtgtgtgggggtctgagtccagcacttctgagtccctcagcctccactcaggtcaggaccagaagtcgctgttcccttctcagggaatagaagattatcccaggtgcctgtgtccaggctggtgtctgggttctgtgctctcttccccatcccgggtgtcctgtccattctcaagatggccacatgcgtgctggtggagtgtcccatgacagatgcaaaatgcctgaattttctgactcttcccgtcagacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatggggtaaggagggagatgggggtgtcatgtctcttagggaaagcaggagcctctctggagacctttagcagggtcagggcccctcaccttcccctcttttcccagagctgtcttcccagcccaccatccccatcgtgggcatcattgctggcctggttctccttggagctgtgatcactggagctgtggtcgctgccgtgatgtggaggaggaagagctcaggtggagaaggggtgaagggtggggtctgagatttcttgtctcactgagggttccaagccccagctagaaatgtgccctgtctcattactgggaagcaccgtccacaatcatgggcctacccagtctgggccctgtgtgccagcacttactcttttgtaaagcacctgttaaaatgaaggacagatttatcaccttgattacggcggtgatgggacctgatcccagcagtcacaagtcacaggggaaggtccctgaggacagacctcaggagggctattggtccaggacccacacctgctttcttcatgtttcctgatcccgccctgggtctgcagtcacacatttctggaaacttctctggggtccaagactaggaggttcctctaggaccttaaggccctggctcctttctggtatctcacaggacattttcttctcacagatagaaaaggagggagttacactcaggctgcaagtaagtatgaaggaggctgatgcctgaggtccttgggatattgtgtttgggagcccatgggggagcccacccacctcacaattcctcctctagccacatcttctgtgggatctgaccaggttctgtttttgttctaccccaggcagtgacagtgcccagggctctgatgtgtccctcacagcttgtaaaggtgagagcttggaggacctaatgtgtgttgggtgttgggcggaacagtggacacagctgtgctatggggtttctttgcattggatgtattgagcatgcgatgggctgtttaaggtgtgacccctcactgtgatggatatgaatttgttcatgaatatttttttctatagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagaccaccccacccccatgtccaccatgaccctcttcccacgctgacctgtgctccctccccaatcatctttcctgttccagagaggtggggctgaggtgtctccatctctgtctcaacttcatggtgcactgagctgtaacttcttccttccctattaaaattagaaccttagtataaatttactttctcaaattcttgccatgagaggttgatgagttaattaaaggagaagattcctaaaatttgagagacaaaataaatggaagacatgagaa

SEQ ID NO: 2 - DNA, 787 bp, synthetic construct
  misc_feature 1..787: note = Description of sequence: Gene: HLA-A
  source 1..787: mol_type = other DNA
  variation 270..514: note = n is any one of a, c, g and t
gctcccactccatgaggtatttcttcacatccgtgtcccggcccggccgcggggagccccgcttcatcgccgtgggctacgtggactacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctactacaaccagagcgaggccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggttctcacaccatccagataatgtatggctgcgacgtggggtcggacgggcgcttcctccgcgggtaccggcaggacgcctacgacggcaaggattacatcgccctgaacgaggacctgcgctcttggaccgcggcggacatggcggctcagatcaccaagcgcaagtgggaggcggcccatgaggcggagcagttgagagcctacctggatggcacgtgcgtggagtggctccgcagatacctggagaacgggaaggagacgctgcagcgcacgg

SEQ ID NO: 3 - DNA, 787 bp, synthetic construct
  misc_feature 1..787: note = Description of sequence: Gene: HLA-A
  source 1..787: mol_type = other DNA
  variation 270..514: note = n is any one of a, c, g and t
gctcccactccatgaggtatttctacacctccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccggaacacacggaaagtgaaggcccagtcacagactgaccgagtggacctggggaccctgcgcggctactacaaccagagcgaggacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggttctcacaccatccagaggatgtatggctgcgacgtggggccggacgggcgcttcctccgcgggtaccagcaggacgcttacgacggcaaggattacatcgccctgaacgaggacctgcgctcttggaccgcggcggacatggcggctcagatcacccagcgcaagtgggagacggcccatgaggcggagcagtggagagcctacctggagggcgcgtgcgtggagtggctccgcagatacctggagaacgggaaggagacgctgcagcgcacgg

SEQ ID NO: 4 - DNA, 787 bp, synthetic construct
  misc_feature 1..787: note = Description of sequence: Gene: HLA-A
  source 1..787: mol_type = other DNA
  variation 270..514: note = n is any one of a, c, g and t
gctcccactccatgaggtatttctacacttccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccggaacacacggaatgtgaaggcccagtcacagactgaccgagtggacctggggaccctgcgcggctactacaaccagagcgaggccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggttctcacaccatccagatgatgtatggctgcgacgtggggtcggacgggcgcttcctccgcgggtaccggcaggacgcctacgacggcaaggattacatcgccctgaaagaggacctgcgctcttggaccgcggcggacatggcagctcagaccaccaagcacaagtgggaggcggcccctgtggcggagcagtggagagcctacctggagggcacgtgcgtggagtggctccgcagatacctggagaacgggaaggagacgctgcagcgcacgg

SEQ ID NO: 5 - DNA, 787 bp, synthetic construct
  misc_feature 1..787: note = Description of sequence: Gene: HLA-A
  source 1..787: mol_type = other DNA
  variation 270..514: note = n is any one of a, c, g and t
gctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggaggcgctgcagcgcacgg

SEQ ID NO: 6 - DNA, 1088 bp, synthetic construct
  misc_feature 1..1088: note = Description of sequence: Gene: HLA-B
  source 1..1088: mol_type = other DNA
atgcgggtcacggcgccccgaaccgtcctcctgctgctctggggggcagtggccctgaccgagacctgggccggctcccactccatgaggtatttctacaccgccatgtcccggcccggccgcggggagccccgcttcattgcagtgggctacgtggacgacacccagttcgtgaggttcgacagcgacgccgcgagtccgaggacggagccccgggcgccatggatagagcaggaggggccggagtattgggaccggaacacacagatcttcaagaccaacacacagacttaccgagagaacctgcggatcgcgctccgctactacaaccagagcgaggccgggtctcacacttggcagacgatgtatggctgcgacgtggggccggacgggcgcctcctccgcgggcataaccagtacgcctacgacggcaaagattacatcgccctgaacgaggacctgagctcctggaccgcggcggacaccgcggctcagatcacccagcgcaagtgggaggcggcccgtgaggcggagcagctgagagcctacctggagggcctgtgcgtggaggggctccgcagacacctggagaacgggaaggagacgctgcagcgcgcggaccccccaaagacacacgtgacccaccaccccgtctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccatcttcccagtccaccatccccatcgtgggcattgttgctggcctggctgtcctagcagttgtggtcatcggagctgtggtcgctactgtgatgtgtaggaggaagagctcaggtggaaaaggagggagctactctcaggctgcgtccagcgacagtgcccagggctctgatgtgtctctcacagcttg

SEQ ID NO: 7 - DNA, 789 bp, synthetic construct
  misc_feature 1..789: note = Description of sequence: Gene: HLA-B
  source 1..789: mol_type = other DNA
  variation 271..516: note = n is any one of a, c, g and t
gctcccactccatgaggtatttccacacctccgtgtcccggcccggccgcggggagccccgcttcatcaccgtgggctacgtggacgacacgctgttcgtgaggttcgacagcgacgccacgagtccgaggaaggagccgcgggcgccatggatagagcaggaggggccggagtattgggaccgggagacacagatctccaagaccaacacacagacttaccgagagagcctgcggaacctgcgcggctactacaaccagagcgaggccgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggtctcaccccctccagagcatgtacggctgcgacgtggggccggacgggcgcctcctccgcgggcataaccagtacgcctacgacggcaaggattacatcgccctgaacgaggacctgcgctcctggaccgccgcggacacggcggctcagatcacccagcgcaagtgggaggcggcccgtgtggcggagcagctgagagcctacctggagggcgagtgcgtggagtggctccgcagatacctggagaacgggaaggagacgctgcagcgcgcgg

SEQ ID NO: 8 - DNA, 3457 bp, synthetic construct
  misc_feature 1..3457: note = Description of sequence: Gene: HLA-B
  source 1..3457: mol_type = other DNA
  variation 2782: note = n is any one of a, c, g and t
tggtgtaggagaagagggatcaggacgaagtcccatgtcccggacggggctctcagggtctcaggctccgagggccgcgtctgcattggggaggcgcagcgttggggattccccactcccacgagtttcacttcttctcccaacctatgtcgggtccttcttccaggatactcgtgacgcgtccccatttcccactcccattgggtgtcgggtgtctagagaagccaatcagtgtcgccggggtcccagttctaaagtccccacgcacccacccggactcagagtctcctcagacaccgagatgcgggtcacggcaccccgaaccctcctcctgctgctctggggggccctggccctgaccgagacctgggccggtgagtgcgggtcgggagggaaatggcctctgtggggaggagcgaggggaccgcaggcgggggcgcaggacccggggagccgcgccgggaggagggtctggcgggtctcagcccctcctcgcccccaggctcccactccatgaggtatttctacaccgccatgtcccggcccggccgcggggagccccgcttcatcgcagtgggctacgtggacgacacgcagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctactacaaccagagcgaggccggtgagtgaccccggcccggggcgcaggtcacgactccccatcccccacgtacggcccgggtcgccccgagtctccgggtccgagatccgcctccctgaggccgcgggacccgcccagaccctcgaccggcgagagccccaggcgcgtttacccggtttcattttcagttgaggccaaaatccccgcgggttggtcggggcggggcggggctcgggggacggggctgaccgcggggccggggccagggtctcacacttggcagacgatgtatggctgcgacctggggccggacgggcgcctcctccgcgggcataaccagttagcctacgacggcaaggattacatcgccctgaacgaggacctgagctcctggaccgcggcggacaccgcggctcagatcacccagcgcaagtgggaggcggcccgtgaggcggagcagctgagagcctacctggagggcacgtgcgtggagtggctccgcagatacctggagaacgggaaggagacgctgcagcgcgcgggtaccaggggcagtggggagccttccccatctcctataggtcgccggggatggcctcccacgagaagaggaggaaaatgggatcagcgctagaatgtcgccctcccttgaatggagaatggcatgagttttcctgagtttcctctgagggccccctcttctctctaggacaattaagggatgacgtctctgaggaaatggaggggaagacagtccctagaatactgatcaggggtcctctttgacccctgcagcagccttgggaaccgtgacttttcctctcaggccttgttctctgcctcacactcagtgtgtttggggctctgattccagcacttctgagtcactttacctccactcagatcaggagcagaagtccctgttccccgctcagagactcgaactttccaatgaataggagattatcccaggtgcctgcgtccaggctggtgtctgggttctgtgccccttccccaccccaggtgtcctgtccattctcaggctggtcacatgggtggtcctagggtgtcctatgagagatgcaaagcgcctgaattttctgactcttcccatcagaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatggggtaaggagggggatgaggggtcatatctcttctcagggaaagcaggagcccttctggagcccttcagcagggtcagggcccctcgtcttcccctcctttcccagagccatcttcccagtccaccatccccatcgtgggcattgttgctggcctggctgtcctagcagttgtggtcatcggagctgtggtcgctactgtgatgtgtaggaggaagagctcaggtagggaaggggtgaggggtggggtctgggttttcttgtcccactgggggtttcaagccccaggtagaagtgttccctgcctcattactgggaagcagcatccacacaggggctaatgcagcctgggaccctgtgtgccagcacttactcttttgtgcagcacatgtgacaatgaaggacggatgtatcaccttgatggttgtggtgttggggtcctgatttcagcattcatgagtcaggggaaggtccctgctaaggacagaccttaggagggcagttggtccaggacccacacttgctttcctcgtgtttcctgatcctgccttgggtctgtagtcatacttctggaaattccttttgggtccaagacgaggaggttcctctaagatctcatggccctgcttcctcccagtcccctcacagnacattttcttcccacaggtggaaaaggagggagctactctcaggctgcgtgtaagtggtgggggtgggagtgtggaggagctcacccaccccataattcctcctgtcccacgtctcctgcgggctctgaccaggtcctgtttttgttctactccagccagcgacagtgcccagggctctgatgtgtctctcacagcttgaaaaggtgagattcttggggtctagagtgggcgggggggggtggggtggggagggggcagaggggaaaggcctgggtaatggagattctttgattgggatgtttcgcgtgtgtggtgggctgtttagagtgtcatcacttaccatgactaaccagaatttgttcatgactgttgttttctgtagcctgagacagctgtcttgtgagggactgagatgcaggatttcttcactcctcccctttgtgacttcaagagcctctggcatctctttctgcaaaggcacctgaatgtgtctgcgtccctgttagcataatgtgaggaggtggagagacagcccacccccgtgtccactgtgacccctgttcgcatgctgacctgtgtttcctccccagaagcttgcggaagggcgaattccagcacactggcggccgttactagtggatccgagctcggtaccaagcttgatgca

SEQ ID NO: 9 - DNA, 4070 bp, synthetic construct
  misc_feature 1..4070: note = Description of sequence: Gene: HLA-B
  source 1..4070: mol_type = other DNA
  variation 3012..3016: note = n is any one of a, c, g and t
gatcaggacgaagtcccaggtcccggacggggctctcagggtctcaggctccgagggccgcgtctgcaatggggaggcgcagcgttggggattccccactcccctgagtttcacttcttctcccaacttgtgtcgggtccttcttccaggatactcgtgacgcatccccacttcccactcccattgggtgtcggatatctagagaagccaatcagcgtcgccggggtcccagttctaaagtccccacgcacccacccggactcagagtctcctcagacgccaagatgctggtcatggcgccccgaaccgtcctcctgctgctctcggcggccctggccctgaccgagacctgggccggtgagtgcgggtcgggagggaaatggcctctgccgggaggagcgaggggaccgcaggcgggggcgcaggacctgaggagccgcgccgggaggagggtcgggcgggtttcagcccctcctcgcccccaggctcccactccatgaggtatttctacacctccgtgtcccggcccggccgcggggagccccgcttcatctcagtgggctacgtggacgacacgcagttcgtgaggttcgacagcgacgccgcgagtccgagagaggagccgcgggcgccgtggatagagcaggaggggccggaatattgggaccggaacacacagatctgcaagaccaacacacagactgaccgagagagcctgcggaacctgcgcggctactacaaccagagcgaggccggtgagtgaccccggcccggggcgcaggtcacgactccccatcccccacggacggcccgggtcgccccgagtctccgggtccgagatccgcctccctgaggccgcgggacccgcccagaccctcgaccggcgagagccccaggcgcgtttacccggtttcattttcagttgaggccaaaatccccgcgggttggtcggggcggggcggggctcggggggactgggctgaccgcgggggcggggccagggtctcacaccctccagtggatgtatggctgcgacgtggggccggacgggcgcctcctccgcgggtataaccagttcgcctacgacggcaaggattacatcgccctgaacgaggacctgagctcctggaccgcggcggacaccgcggctcagatcacccagcgcaagtgggaggcggcccgtgaggcggagcagcggagagcctacctggagggcacgtgcgtggagtggctccgcagacacctggagaacgggaaggagacgctgcagcgcgcgggtaccaggggcagtggggagccttccccatctcctataggtcgccggggatggcctcccacgagaagaagaggaaaatgggatcagcgctagaatgtcgccctcccttgaatggagaatggcatgagttttcctgagtttcctctgagggccccctcttctctctaggacaattaagggatgacgtctctgaggaaatggaggggaagacagtccctagaatactgatcaggggtcccctttgacccctgcagcagccttgggaaccatgacttttcttctcaggccttgttctctgcctcacactcagtgtgtttggggctctgattccagcacttctgagtcactttacctccactcagatcaggagcagaagtctctgttccccgctcagagactcgaactttccaatgaatagattatcccaggtgcctgcgtccaggctggtgtctgggttctgtgtcccttccccaccccaggtgtcctgtccattctcaggctggtcacatgggtggtcctagggtgtcccatgagagatgcaaagcgcctgaattttctgactcttcccatcagaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagacagaaccttccagaagtgggcagctgtggtggtgccttctggagaagagcagagatacacatgccatgtacagcatgaggggctgccgaagcccctcaccctgagatggggtaaggagggggatgaggggtcatatctcttctcagggaaagcaggagcccttcagcagggtcagggcccctcatcttcccctcctttcccagagccatcttcccagtccaccgtccccatcgtgggcattgttgctggcctggctgtcctagcagttgtggtcatcggagctgtggtcgctgctgtgatgtgtaggaggaagagttcaggtagggaaggggtgaggggtggggtctgggttttcttgtcccactgggggtttcaagccccaggtagaagtgttccctgcatcattactgggaagcagcatgcacacaggggctaacgcagcctgggaccctgtgtgccagcacttactcttttgtgcagcacatgtgacaatgaaggacggatgtatcaccttgatggttgtggtgttggggtcctgattccagcattcatgagtcaggggaaggtccctgctaaggacagaccttaggagggcagttggtccaggacccacacttgctttcctcgtgtttcctgatcctgccctgggtctgtagtcatacttctggaaattccttttgggtccaagactaggaggttcctctaagatctcatggccctgcttcctcccagtcccctcacaggacattttcttcccacaggtggaaaaggagggagctactctcaggctgcgtgtaagtggtgggggtgggagtgtggaggagctcacccaccccataattcctcctgtcccacgtctcctgtgggctctgaccaggtcctgtttttgttctactccagccagcgacagtgcccagggctctgatgtgtctctcacagcttgaaaaggtgagattcttggggtctagagtgggcgggggggcggggagggggcagnggggaaaggcctgggtaatggagattctttgattgggatgtttcgcgtgtgtgatgggctgttcagagtgtcatcacttaccatgactaaccagaatttgttcatgactgttgttttctgtagcctgagacagctgtcttgtgagggactgagatgcaggatttcttcacgcctcccctttgtgacttcaagagcctctggcatctctttctgcaaaggcacctgaatgtgtctgcgtccctgttagcataatgtgaggaggtggagagacagcccacccttgtgtccactgtgacccctgttcccatgctgacctgtgtttcctccccagtcatctttcttgttccagagaggtggggctggatgtctccatctctgtctcaactttatatgcactgagctgcaacttcttacttccctactgaaaataagaatctgaatataaatttgttttctcaaatatttgctatgagaggttgatggattaattaaataagtcaattcctggaatttgagagagcaaataaagacctgagaaccttccagaatctgcatgttcgctgtgctgagtctgttgcaggtggggtgtggagaaggctgtggggggccaagtgtggacggggcctgtgcccatttggtgttgagtccatcatgggctttatgtggttagtcctcagttgggtcaccttcactgctccattgtccttgtcccttcagtggaaacttgtccagtgggagctgtgaccacagaggctcacacatcgcccagggcggcccctgcacacgggggtctctgtgcattctgagacaaattttcagagccattcacctcctgccctgcttctagagctccttttctgctctgctcttctgccctctctccctgccctggttctagtgatcttggtgctgaatccaatcccaactcatgaatctgtaaagcagagtctaatttagacttacatttgtctgtgaaattggacccatcatcaaggactgttctttcctgaagagagaacctgattgtgtgctgcagtgtgctggggcagggggtgcgg

SEQ ID NO: 10 - DNA, 2065 bp, synthetic construct
  misc_feature 1..2065: note = Description of sequence: Gene: HLA-B
  source 1..2065: mol_type = other DNA
atgcgggtcacggcgccccgaaccgtcctcctgctgctctggggggcagtggccctgaccgagacctgggccggtgagtgcggggtcgggagggaaatggcctctgtggggaggagcgaggggaccgcaggcgggggcgcaggacctgaggagccgcgccgggaggagggtcgggcgggtctcagcccctcctcgcccccaggctcccactccatgaggtatttctacaccgccatgtcccggcccggccgcggggagccccgcttcatcgcagtgggctacgtggacgacacccagttcgtgaggttcgacagcgacgccgcgagtccgaggacggagccccgggcgccatggatagagcaggaggggccggagtattgggacggggagacgcggaacatgaaggcctccgcgcagacttaccgagagaacctgcggatcgcgctccgctactacaaccagagcgaggccggtgagtgaccccggcccggggcgcaggtcacgactccccatcccccacgtacggcccgggtcgccccgagtctccgggtccgagatccgcctccctgaggccgcgggacccgcccagaccctcgaccggcgagagccccaggcgcgtttacccggtttcattttcagttgaggccaaaatccccgcgggttggtcggggcggggcggggctcgggggacggggctgaccgcggggccggggccagggtctcacatcatccagaggatgtatggctgcgacctggggcccgacgggcgcctcctccgcgggcatgaccagtccgcctacgacggcaaggattacatcgccctgaacgaggacctgagctcctggaccgcggcggacaccgcggctcagatcacccagcgcaagtgggaggcggcccgtgtggcggagcagctgagagcctacctggagggcctgtgcgtggagtggctccgcagatacctggagaacgggaaggagacgctgcagcgcgcgggtaccaggggcagtggggagccttccccatctcctataggtcgccggggatggcctcccacgagaagaggaggaaaatgggatcagcgctagaatgtcgccctcccttgaatggagaatggcatgagttttcctgagtttcctctgagggccccctcttctctctaggacaattaagggatgacgtctctgaggaaatggaggggaagacagtccctagaatactgatcaggggtcccctttgacccctgcagcagccttgggaaccgtgacttttcctctcaggccttgttctctgcctcacactcagtgtgtttggggctctgattccagcacttctgagtcactttacctccactcagatcaggagcagaagtccctgttccccgctcagagactcgaactttccaatgaataggagattatcccaggtgcctgcgtccaggctggtgtctgggttctgtgccccttccccacaccaggtgtcctgtccattctcaggctggtcacatgggtggtcctagggtgtcccatgagagatgcaaagcgcctgaattttctgactcttcccatcagaccccccaaagacacacgtgacccaccaccccgtctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatggggtaaggagggggatgaggggtcatatctcttctcagggaaagcaggagcccttctggagcccttcagcagggtcagggcccctcgtcttcccctcctttcccagagccatcttcccagtccaccatccccatcgtgggcattgttgctggcctggctgtcctagcagttgtggtcatcggagctgtggtcgctactgtgatgtgtaggaggaagagctcag

SEQ ID NO: 11 - DNA, 1621 bp, synthetic construct
  misc_feature 1..1621: note = Description of sequence: Gene: HLA-C
  source 1..1621: mol_type = other DNA
  variation 74..174: note = n is any one of a, c, g and t
  variation 444..543: note = n is any one of a, c, g and t
  variation 820..919: note = n is any one of a, c, g and t
  variation 1196..1295: note = n is any one of a, c, g and t
  variation 1496..1515: note = n is any one of a, c, g and t
  variation 1549..1558: note = n is any one of a, c, g and t
  variation 1607..1616: note = n is any one of a, c, g and t
atgcgggtcatggcgccccgaaccctcctcctgctgctctcgggagccctggccctgaccgagacctgggcctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngctcccactccatgaggtatttctacaccgctgtgtcccggcccggccgcggagagccccacttcatcgcagtgggctacgtggacgacacgcagttcgtgcggttcgacagcgacgccgcgagtccaagaggggagccgcgggcgccgtgggtggagcaggaggggccggagtattgggaccgggagacacagaactacaagcgccaggcacagactgaccgagtgaacctgcggaaactgcgcggctactacaaccagagcgaggccgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggtctcacatcatccagaggatgtatggctgcgacctggggcccgacgggcgcctcctccgcgggcatgaccagttagcctacgacggcaaggattacatcgccctgaacgaggacctgcgctcctggaccgccgcggacacggcggctcagatcacccagcgcaagtgggaagcggcccgtgaggcggagcagctgagagcctacctggagggcacgtgcgtggagtggctccgcagatacctggagaacgggaaggagacgctgcagcgcgcggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaacacccaaagacacacgtgacccaccatcccgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccggagcccctcaccctgagatgggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagccatcttcccagcccaccatccccatcgtgggcatcgttgctggcctggctgtcctggctgtcctagctgtcctaggagctgtgatggctgttgtgatgtgtaggaggaagagctcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtggaaaaggagggagctgctctcaggctgcgtnnnnnnnnnnccagcaacagtgcccagggctctgatgagtctctcatcgcttgtaaagnnnnnnnnnncctga

SEQ ID NO: 12 - DNA, 3350 bp, synthetic construct
  misc_feature 1..3350: note = Description of sequence: Gene: HLA-C
  source 1..3350: mol_type = other DNA
aggagaagagggatcaggacgaagtcccaggtcccgggcggggctctcagggtctcaggctccaagggccgtgtctgcactggggaggcgccgcgttgaggattctccactcccctgagtttcacttcttctcccaacctgcgtcgggtccttcttcctgaatactcatgacgcgtccccaattcccactcccattgggtgtcgggttctagagaagccaatcagcgtctccgcagtcccggttctaaagtccccagtcacccacccggactcggattctccccagacgccgagatgcgggtcatggcgccccgaaccctcatcctgctgctctcgggagccctggccctgaccgagacctgggcctgtgagtgcggggttgggagggaaacggcctctgcggagaggagcgaggggcccgcccggcgagggcgcaggacccggggagccgcgcagggaggagggtcgggcgggtctcagcccctcctcgcccccaggctcccactccatgaggtatttctacaccgccgtgtcccggcccggccgcggagagccccgcttcatcgcagtgggctacgtggacgacacgcagttcgtgcggttcgacagcgacgccgcgagtccaagaggggagccgcgggcgccgtgggtggagcaggaggggccggagtattgggaccgggagacacagaagtacaagcgccaggcacagactgaccgagtgaacctgcggaaactgcgcggctactacaaccagagcgaggccggtgagtgaccccggcccggggcgcaggtcacgacccctccccatcccccacggacggcccgggtcgccccgagtctcccggtctgagatccaccccgaggctgcggaacccgcccagaccctcggccggagagagccccagtcacctttacccggtttcattttcagtttaggccaaaatccccgcgggttggtcggggctggggcggggctcgcgggacggggctgaccacgggggcggggccagggtctcacaccctccagtggatgtatggctgcgacctggggcccgacgggcgcctcctccgcgggtatgaccagtccgcctacgacggcaaggattacatcgccctgaacgaggacctgcgctcctggaccgccgcggacacggcggctcagatcacccagcgcaagtgggaggcggcccgtgcggcggagcagcagagagcctacctggagggcacgtgcgtggagtggctccgcagatacctggagaacgggaaggagacgctgcagcgcgcgggtaccaggggcagtggggagccttccccatctcctgtagatctcccgggatggcctcccacgaggaggggaggaaaatgggatcagcgctagaatatcgccctcccttgaatggagaatgggatgagttttcctgagtttcctctgagggccccctctgctctctaggacaattaagggatgaagtccttgaggaaatggaggggaagacagtccctggaatactgatcaggggtcccctttgaccactttgaccactgcagcagctgtggtcaggctgctgacctttctctcaggccttgttctctgcctcatgctcaatgtgtttgaaggtttgattccagcttttctgagttcttcagcctccactcaggtcaggaccagaagtcgctgttcctccctcagagactagaactttccaatgaataggagattatcccaggtgcctgtgtccaggctggcgtctgggttctgtgcccccttccccaccccaggtgtcctgtccattctcaggatggtcacatgggcgctgttggagtgtcgcaagagagatacaaagtgtctgaattttctgactcttcccgtcagaacacccaaagacacacgtgacccaccatctcgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccggagcccctcaccctgagatggggtaaggagggggatgaggggtcatgtgtcttctcagggaaagcagaagtcctggagcccttcagccgggtcagggctgaggcttgggggtcagggcccctcaccttcccctcctttcccagagccatcttcccagcccaccatccccatcgtgggcatcgttgctggcctggctgtcctggctgtcctagctgtcctaggagctgtggtggctgttgttatgtgtaggaggaagagctcaggtagggaaggggtgaggagtggggtctgggttttcttgtcccactgggagtttcaagccccaggtagaagtgtgccccacctcgttactggaagcaccatccacacatgggccatcccagcctgggaccctgtgtgccagcacttactctgttgtgaagcacatgacaatgaaggacagatgtatcaccttgatgattatggtgttggggtccttgattccagcattcatgagtcaggggaaggtccctgctaaggacagaccttaggagggcagttgctccagaacccacagctgctttccctgtgtttcctgatcctgccctgggtctgcagtcatagttctggaaacttctcttgggtccaagactaggaggttcccctaagatcgcatggccctgcctcctccctgtcccctcacagggcattttcttcccacaggtggaaaaggagggagctgctctcaggctgcgtgtaagtgatggcagtgggcgtgtggaggagctcacccaccccataattcctcttgtcccacatctcctgcgggctctgaccaggtctttttttttgttctaccccagccagcaacagtgcccagggctctgatgagtctctcatcgcttgtaaaggtgagattctggggagctgaagtggtcgggggtggggcagagggaaaaggcctaggtaatggggatcctttgattgggacgtttcgaatgtgtggtgagctgttcagagtgtcatcacttaccatgactgacctgaatttgttcatgactattgtgttctgtagcctgagacagctgcctgtgtgggactgagatgcaggatttcttcacacctctcctttgtgacttcaagagcctctggcatctctttctgcaaaggcacctgaatgcgtctgcgttcctgttagcataatgtgaggaggtggagagacagcccacccccgtgtccac

SEQ ID NO: 13 - DNA, 3350 bp, synthetic construct
  misc_feature 1..3350: note = Description of sequence: Gene: HLA-C
  source 1..3350: mol_type = other DNA
aggagaagagggatcaggacgaagtcccaggtcccgggcggggctctcagggtctcaggctccaagggccgtgtctgcactggggaggcgccgcgttgaggattctccactcccctgagtttcacttcttctcccaacctgcgacgggtccttcttcctgaatactcatgacgcgtccccaattcccactcccattgggtgtcgggttctagagaagccaatcagcgtctccgcagtcccggttctaaagtccccagtcacccacccggactcggattctccccagacgccgagatgcgggtcatggcgccccgaaccctcctcctgctgctctcgggagccctggccctgaccgagacctgggcctgtgagtgcggggttgggagggaaacggcctctgcggagaggagcgaggggcccgcccggcgagggcgcaggacccggggagccgcgcagggaggagggtcgggcgggtctcagcccctcctcgcccccaggctcccactccatgaggtatttctacaccgctgtgtcccggcccggccgcggagagccccacttcatcgcagtgggctacgtggacgacacgcagttcgtgcggttcgacagcgacgccgcgagtccaagaggggagccgcgggcgccgtgggtggagcaggaggggccggagtattgggaccgggagacacagaactacaagcgccaggcacagactgaccgagtgaacctgcggaaactgcgcggctactacaaccagagcgaggccggtgagtgaccccggcccggggcgcaggtcacgacccctccccatcccccacggacggcccgggtcgccccgagtctcccggtctgagatccaccccgaggctgcggaacccgcccagaccctcgaccggagagagccccagtcacctttacccggtttcattttcagtttaggccaaaatccccgcgggttggtcggggctggggcggggctcgggggacggggctgaccacgggggcggggccagggtctcacatcatccagaggatgtatggctgcgacctggggcccgacgggcgcctcctccgcgggcatgaccagttagcctacgacggcaaggattacatcgccctgaacgaggacctgcgctcctggaccgccgcggacacggcggctcagatcacccagcgcaagtgggaggcggcccgtgaggcggagcagctgagagcctacctggagggcacgtgcgtggagtggctccgcagatacctggagaacgggaaggagacgctgcagcgcgcgggtaccaggggcagtggggagccttccctatctcctgtagatctcccgggatggccttccacgaggaggggaggaaaatgggatcagcgctagaatatcgccctcccttgaatggagaatgggatgagttttcctgagtttcctctgagggccccctctgctctctaggacaattaagggatgaagtccttgaggaaatggaggggaagacagtccctggaatactgatcaggggtcccctttgaccactttgaccactgcagcagctgtggtcaggctgctgacctttctctcaggccttgttctctgcctcacgctcaatgtgtttgaaggtttgattccagcttttctgagtccttcggcctccactcaggtcaggaccagaagtcgctgttcctccctcagagactagaactttccaatgaataggagattatcccaggtgcctgtgtccaggctggcgtctgggttctgtgcccccttccccaccccaggtgtcctgtccattctcaggatggtcacatgggcgctgttggagtgtcgcaagagagatacaaagtgtctgaattttctgactcttcccgtcagaacacccaaagacacacgtgacccaccatcccgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccggagcccctcaccctgagatggggtaaggagggggatgaggggtcatgtgtcttctcagggaaagcagaagtcctggagcccttcagctgggtcagggctgaggcttgggggtcagggcccctcaccttcccctcctttcccagagccatcttcccagcccaccatccccatcgtgggcatcgttgctggcctggctgtcctggctgtcctagctgtcctaggagctgtgatggctgttgtgatgtgtaggaggaagagctcaggtagggaaggggtgaggagtggggtctgggttttcttgtcccactgggagtttcaagccccaggtagaagtgtgccccacctcgttactggaagcaccatccacacatgggccatcccagcctgggaccctgtgtgccagcacttactctgttgtgaagcacatgacaatgaaggacagatgtatcaccttgatgattatggtgttggggtccttgattccagcattcatgagtcaggggaaggtccctgctaaggacagaccttaggagggcagttgctccagaacccacagctgctttccccgtgtttcctgatcctgccctgggtctgcagtcatagttctggaaacttctcttgggtccaagactaggaggttcccctaagatcgcatggccctgcctcctccctgtcccctcacagggcattttcttcccacaggtggaaaaggagggagctgctctcaggctgcgtgtaagtgatggcggtgggcgtgtggaggagcttacccaccccataattcctcttgtcccacatctcctgcaggctctgaccaggtctttttttttgttctaccccagccagcaacagtgcccagggctctgatgagtctctcatcgcttgtaaaggtgagattctggggagctgaagtggtcgggggtggggcagagggaaaaggcctaggtaatggggatcctttgattgggacgtttcgaatgtgtggtgagctgttcagagtgtcatcacttaccatgactgacctgaatttgttcatgactattgtgttctgtagcctgagacagctgcctgtgtgggactgagatgcaggatttcttcacacctctcctttgtgacttcaagagcctctggcatctctttctgcaaaggcatctgaatgtgtctgcgttcctgttagcataatgtgaggaggtggagagacagcccacccccgtgtccac

SEQ ID NO: 14 - DNA, 3355 bp, synthetic construct
  misc_feature 1..3355: note = Description of sequence: Gene: HLA-C
  source 1..3355: mol_type = other DNA
aggagaagagggatcaggacgaagtcccaggtcccgggcggggctctcagggtctcaggctccaagggccgtgtctgcattggggaggcgccgcgttggggattctccactcccctgagtttcacttctcccaacctgcgtcgggtccttcttcctgaatactcatgacgcgtccccaattcccactcccattgggtgtcgggttctagagaagccaatcagcgtctccgcagtcccggttctaaagtccccagtcacccacccggactcacattctccccagaggccgagatgcgggtcatggcgccccgagccctcctcctgctgctctcgggaggcctggccctgaccgagacctgggcctgtgagtgcggggttgggagggaagcggcctctgcggagaggagcgaggggccctcccggcgagggcgcaggacccggggagccgcgcagggaggtgggtcgggcgggtctcagcccctcctcgcccccaggctcccactccatgaggtatttcgacaccgccgtgtcccggcccggccgcggagagccccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacaggctgaccgagtgagcctgcggaacctgcgcggctactacaaccagagcgaggacggtgagtgaccccggcccggggcgcaggtcacgacccctccccatcccccacggacggcccgggtcgcccagagtctccccgtctgagatccaccccaaggtggatctgcggaacccgcccagaccctcgaccggagagagccccagtcgcctttacccggtttcattttcggtttaggccaaaatccccgcgggttggtcggggcggggcggggctcgggggactgggctgaccgcgggggcggggccagggtctcacaccctccagaggatgtctggctgcgacctggggcccgacgggcgcctcctccgcgggtatgaccagtccgcctacgacggcaaggattacatcgccctgaacgaggacctgcgctcctggaccgccgcggacaccgcggctcagatcacccagcgcaagttggaggcggcccgtgcggcggagcagctgagagcctacctggagggcacgtgcgtggagtggctccgcagatacctggagaacgggaaggagacgctgcagcgcgcaggtaccaggggcagtggggagccttccccatctcctatagatctcccgggatggcctcccacgaggaggggaggaaaatgggatcagcactggaatatcgccctcccttgaatggagaatggcatgagttttcctgagtttcctctgagggccccctctgctctctaggacaattaagggatgaagtctctgaggaaatggaggggaagacagtccctggaatactgatcaggggtctcctttgaccactttgaccactgcagcagctgtggtcaggctgctgacctttctctcaggccttgttctctgcctcacactcaatgtgtctgaaggtttgattccagcttttctgagtcctgcagcctccactcaggtcaggaccagaagtcgctgttcctccctcagagactagaactttccaatgaataggagattatcccaggtgcctgtgtccaggctggcgtctgggttctgtgccgccttccccaccccaggtgtcctgtccattctcaggatggtcacatgggcgctgctggagtgtcccaagagagatgcaaagtgtctgaattttctgactcttcccgtcagaacccccaaagacacacgtgacccaccaccccctctctgaccatgaggccaccctgaggtgctgggccctgggcttctaccctgcggagatcacactgacctggcagcgggatggggaggaccagacccaggacaccgagcttgtggagaccaggccagcaggagatggaaccttccagaagtgggcagctgtggtggtgccttctggacaagagcagagatacacgtgccatatgcagcacgaggggctgcaagagcccctcaccctgagctggggtaaggaggggaatggggggtcacatctcttatcagagaaagcagaagtccttctggagcccttcagccgggtcagggctgaggcttgggggtcagggcccctcaccttctcctcctttcccagagccatcttcccagcccaccatccccatcatgggcatcgttgctggcctggctgtcctggttgtcctagctgtccttggagctgtggtcaccgctatgatgtgtaggaggaagagctcaggtagggaaggggtgaagagcggggtctgggttttcttgtcccactgggagtttcaagccccaggtagaagtgtgccccgccttgttactggaagcaccatccacacatgggccatcccagcctgggaccctgtgtgccagcacttactcttttgtgaagcacatgtgacaatgaaggacggatgtatcaccttgatgattatggtgttggggtcctgattccagcattcatgagtcaggggaaggtccctgctaaggacagaccttaggagggcagttggtccagaacccacaactgctttccccatgtttcctgatcctgccctgggtctgcagtcgtagttctggaaacttctcttgggtccaagactaggaggttcccctaagatcacatggccctgcctcctcccagtcccctcatagggcattttcttcccacaggtggaaaaggagggagctgctctcaggctgcgtgtaagtgatggcggcgggcgtgtggaggagctcacctactccataattcctcttgtcccacatctcctgcgggctctgaccaggtctttttttttgttctaccccaggcagcaacagtgcccagggctctgatgagtctctcatcacttgtaaaggtgagattctggggagctgaagtggtcgggggtggggcagagggaaaaggcctgggtaatggggattctttgattgggacgtttcgagtgtgtggtgggccgttcagagtgtcatcgcttaccatgactgacctgaatttgttcatgactattgtgttctgtagcctgagacagctgcctgtgtgggactgagatgcaggatttcttcacacctctcctttgtgacttcaagagcctctggcatctctttctgcaaaggcacctgaatgtgtctgcgtccctgttagcataatgtgaggaggtggagagacagcccacccccgtgtccac

SEQ ID NO: 15 - DNA, 3421 bp, synthetic construct
  misc_feature 1..3421: note = Description of sequence: Gene: HLA-C
  source 1..3421: mol_type = other DNA
tcaggcacacagtgtgacaaagatgcttggtgtaggagaagaggaatcaggacgaagtcccaggtcccgggcggggctctcagggtctcaggctccaagggccgtgtctgcactggggaggcgccgcgttggggattctccactcccctgagtttcacttcttctcccaacctgcgtcgggtccttcttcctgaatactcatgacgcgtccccaattcccactcccattgggtgtcgggttctagagaagccaatcagcgtctccgcagtcccggttctaaagtccccagtcacccacccggactcagattctccccagacgccgagatgcgggtcatggcgccccgaaccctcatcctgctgctctcgggagccctggccctgaccgagacctgggccggtgagtgcggggttgggagggaaacggcctctggggagaggagcgaggggcccgcccggcgagggcgcaggacccggggagccgcgcagggaggagggtcgggcgggtctcagccactcctcgtccccaggctcccactccatgaggtatttctccacatccgtgtcctggcccggccgcggggagccccgcttcatcgcagtgggctacgtggacgacacgcagttcgtgcggttcgacagcgacgccgcgagtccaagaggggagccgcgggagccgtgggtggagcaggaggggccggagtattgggaccgggagacacagaagtacaagcgccaggcacaggctgaccgagtgaacctgcggaaactgcgcggctactacaaccagagcgaggacggtgagtgaccccggcccggggcgcaggtcacgacccctccccatcccccacggacggcccgggtcgccccgagtctccccgtctgagatccaccccgaggctgcggaacccgcccagaccctcgaccggagagagccccagtcacctttacccggtttcattttcagtttaggccaaaatccccgcgggttggtcgggactggggcggggctcgggggaccgggctgaccacgggggcggggccagggtctcacaccctccagaggatgtttggctgcgacctggggccggacgggcgcctcctccgcgggtataaccagttcgcctacgacggcaaggattacatcgccctgaacgaggatctgcgctcctggaccgccgcggacacggcggctcagatcacccagcgcaagtgggaggcggcccgtgaggcggagcagcggagagcctacctggagggcacgtgcgtggagtggctccgcagatacctggagaacgggaaggagacgctgcagcgcgcgggtaccaggggcagtggggagccttccccatctcccgtagatctcccgggatggcctcccacgaggaggggaggaaaatgggatcagcgctagaatatcgccctcccttgaatggagaatgggatgagttttcctgagtttcctctgagggccccctctgctctctaggacaattaagggatgaagtccttgaggaaatggaggggaagacagtccctggaatactgatcaggggtcccctttgaccactttgaccactgcagcagctgtggtcaggctgctgacctttctctcaggccttgttctctgcctgacgctcaatgtgtttgaaggtttgattccagcttttctgagtccttcggcctccactcaggtcaggaccagaagtcgctgttcctccctcagagactagaactttccaatgaataggagattatcccaggtgcctgtgtccaggctggcgtctgggttctgtgcccccttccccaccccaggtgtcctgtccattctcaggatggtcacatgggcgctgttggagtgtcgcaagagagatacaaagtgtctgaattttctgactcttcccatcagaacacccaaagacacacgtgacccaccatcccgtctctgaccatgaggccaccctgaggtgctgggccctgggcttctaccctgcggagatcacactgacctggcagtgggatggggaggaccaaactcaggacaccgagcttgtggagaccaggccagcaggagatggaaccttccagaagtgggcagctgtggtggtgccttctggagaagagcagagatacacgtgccatgttcagcacgaggggctgccggagcccctcaccctgagatggagtaaggagggggatgaggggtgatgtgtcttctcagggaaagcagaagtcctggagcccttcagccgggtcagggctgaggcttggaggtcagggcccctcaccttcccctcctttcccagagccgtcttcccagcccaccatccccatcgtgggcatcgttgctggcctggctgtcctggctgtcctagctgtcctaggagctatggtggctgttgtgatgtgtaggaggaagagctcaggtagggaaggggtgaggagtggggtctgggttttcttgttccactgggagtttcaagccccaggtagaagtgtgccccacctcgttactggaagcaccatccacacatgggccatcccagcctgggaccctgtgtgccagcacttactctgttgtgaagcacatgacaatgaaggacagatgtatcaccttgatgattatggtgttggggtccttgattccagcattcatgagtcaggggaaggtccctgctaaggacagaccttaggagggcagttgcttcagaacccacagctgctttccccgtgtttcctgatcctgccctgggtctgcagtcatagttctggaaacttctcttgggtccaagactaggaggttcccctaagatcgcatggccctgcctcctccctgtcccctcacagggcattttcttcccacaggtggaaaaggagggagctgctctcaggctgcgtgtaagtgatggcggtgggcgtgtggaggagctcacccaccccataattcctcttgtcccacatctcctgcgggctctgaccaggtctttttttttgttctaccccagccagcaacagtgcccagggctctgatgagtctctcatcgcttgtaaaggtgagattctggggagctgaagtggtcgggggtggggcagagggaaaaggcctaggtaatggggatcctttgattgggacgtttcgaatgtgtggtgagctgttcagagtgtcatcacttaccatgactgacctgaatttgttcatgactattgtgttctgtagcctgagacagctgcctgtgtgggactgagatgcaggatttcttcacacctctcctttgtgacttcaagagcctctggcatctctttctgcaaaggcatctgaatgtgtctgcgttcctgttagcataatgtgaggaggtggagagacagcccaccccggtgtccaccgtgacccctgtccccacactgacctgtgttccctccc

SEQ ID NO: 16 - DNA, 5396 bp, synthetic construct
  misc_feature 1..5396: note = Description of sequence: Gene: Beta-2M
  source 1..5396: mol_type = other DNA
tttcagtggctgctactcggcgcttcagtcgcggtcgcttcagtcgtcagcatggctcgctcggtgaccctggtctttctggtgcttgtctcactgaccggcctgtatgctatccagagtgagtgcctctttcccctctcttggcattaaatttttagttctccttagttctccttcccgacgggattggccagggctgcgtctctcgggaaaggagtgggcgtcccgctgcttacagctttgaggctacagggtggatgcgcctgtttgcgagtgtgctgacggtcagcgctgaagatggggaaggcttctctttttctcctctgctggcgggagcccctagactccctgagcgcaaaaggagggtggggctgcctggaaagtgcagggtggagggctatgggcacgcggaagctgagccccgaccacgggaagtcagaccgccgctgcgcagcttcatttgcactcgcagagaggctcgatccaaccgaagtcgagagaaaatgggcttgggtcccagactctgcgatgtttccaaagctttctgatcattaactttgtatctagtaataaccttaaaggtcgccgggcagtggtggctcacgcctttcatgccagcacttgggaggcagaggtaggcggatttctgaattcgaggccagcctggtctacagagtgagttccaggacagccagggttacacaaagaaaccctgtctcgaaaaaataaataaaggtctctgggtatgaggcttattgcaatgctgaggaccttgtgagcttcttaagtttgaaagtacgtctacaatgtaaatgttatggttccccgtgtattaagacagggaactagaggaggcatcgagatttaaagtaacttattcacacgatagactaatggcagtcggagctggaattgaggccgatcaatgcccaggcctttggactgtaaccgccatttgtttctagaagtgtctaggccttcctgggtcatggtctgtgaagcagtctgagatgccttggacccttggtacctcacacagcgcttcctttttggcacactgcctggttctttccgcgatagagcctctgctttcagttttgagacaactaagactttgccttaagtcaaggtggttatgaactctggagtgaggattatttcagtgtggtgaccaagactcgtgaggataactagtaactaccaaagtcgtggaggtagaaatatggcagagaccagatttaaacctgacccaacactggacttaattgtggaagggatggttcttgtccagagaattccaggcatgatgaacctgtggcatttagcatgttcttgtttccaaaacctcaaatgcaaggaaaaatgtggtttatgctaatttaaatttatggggacaaaaagaattcaaagctttactgagaccttcattctagctggccacagttttcaggttttcctctccctactgccttccatgggtgaataagaggctgctcttcagagaccgtgcacactgaatagatgtgtatctgggagtatattatggctgggcatagttgtgcacatctataatctcagtatttaggagactgaggcaggtggatctctgaatttgagaccaacctgatctacaaagtgcatccaggacagaaaaacgctgtcttgaaaaaccaaagaaagaaagaaagaaagaaaagaaagtgttttataagccaagtgtgttgagtcatgctgtgacctcagagctggggaggctgagacaggcgttcaaggccagcctggaagacagtcagactgtatctcaaattaaataataagcatataagtaaatgaaggggaaaacacaaaaagagccagtgaaatggctcagcaggtacaggcgcttaccactaagcctgagtgtatctcagatcccacttggtggacggagagaactgacttttaaatccacacatgagctacgtttcgtgcatgtgcacacacacacacacacacacacacacacacacacacacatatatactctctcagtaaaacaataatttttttaagaaagtagatatatacattagtgataccttggtatcagatctgcatattctacacagtagtactccttgtcatgttggttgagaagcagaaacagagttcaaagatggcagtggtatggctcagcagctaaaagtacctgccatgcaacctgaccacctgagttcatagaacccacgacagaaggaggagaaaactgtctcctgaaagccatccaatgtcttcacaagtacacctttgggaacttgactgtgtgtatgtgtgtgtgcacatacaaataacaatggaaagagtttacattattttctttccatgtccctatagcgtgcagtagctattcccaacatgttgagcgacttcagctatctgaaagccccatatccccttgcacacactgcttacttccagcagagtgcattctcaattgtcatggtcctcacatctccctctggtcctttacccatcccaactcagttgcttaaagctttcctagcttctgtggtattttaaattttgtttgttttctagttcatgaaaccagaggtttggtttcagaatgcaaactctgggctggaacagagaaaccctgggttgactcgcataggaacctcattagggaggagccaatgctaacacctgccacctgaggggtaattgctcagctctcagcactggatcagacatatgtgttgggaagtctagggaggagcaataagcaaaaacgaaaggggatgaaaataagaaagaaaagtgagaggggctgacacagcgcacaagcactgcgggctttgatgtggatactgtgaagggtgtgcagaatgggatgtgacgtttggaaagttggtgggatttattaagggaagggaggggcacagttcttgagagcttccaataataaaagctccttaaaaatccactgacagaagacactgctaaaagccaggtagggaaaacagaaggtactcgtaggattttgaggaaaagatactacgttttcaaaatgtgggtagactttgggggaagcagatcacttatccagagtagaaatggaacagggagaaatagaggaacaaatgtaagatggtgcacggtgcagactgagctctgttttcatctgtcttcccctgtggccctcagaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactggggtaagcctcaagttcttccttactttctggacgctccatctgtgtggacttaaaactgctttgctattttaaaaacctgcatactgagattgttagagaatacccccaaaagactgatctgtggtgccagcagagacttacacagcaggccaggaaccagcagttccctacccactgtcccagactgcttccctctttcctgagcttcctccagctttcctggcagcctggtgcatactaagtgtcaaagggcatgccaggtaggacaggccttggtctgtaacccctgccttcaggtacctacaattctggactgttccagggccttggcaagaagctagtagacaggagtggatctctggaaagttctcaacaagtactcaaggctagcaacagcaacctagtctatctgccttactgcttacaacccctggatgtctgttaatatgtcgtagtataaaagtaaagttttgtctccattttcttttttcatagatcgagacatgtgatcaagcatcatgatggtaggtttcttgactttaaataatgggcctttgggggtggggtggagctggcggtagagcacatgcctagcgtgcataaggccttgagtcctggtcccagcactgcaggaagagaattgtttgattcactatcctaggagctgtttatagacagctcaaacatgataagcatcactgtatgaaagacattaattagggtggcttgatcagctacagaaggatcctttggggtcacattcttttatcctgtggactggcaggaaggaggaacgtagccatgtcactggccctctaaagggagaacagcacctttgtgcatgcagcaaggcaggtaggcaggtaggcaggtaggcaggcgggtggtcagttacacagcctgcatgggtttgccgaccttctgttttaaccctgccagttctgctgagaaggcctgggacgatgagcttgctgtctttgtacagcagtggccttgctcctcacccagaggagccgagtgacagagctctgcgggtacatcttagccctttcagtctctagttggtaggcactagatttaccttctggaggcttccggacactcagggaaagaaatggacagggttgtaacttcatgtaaggcaccgtcactgatgtgtcagaaggaagttgaggagcgtgagagggaacgtgggtgtctctgtcaggtggagtctagtggtagaaaatccagcttttcggtgaaatccagggcccttgaggccaaaagctcactcaaatcttgtattatatttatttctaggaaaggggaattgatgaagggggtggggatgggtgatctgcccagacaagcagttaccaaatggtggagccagaactcacctcctttaagaaggtgattgtcctacaaactcatttgatggtgaggtctggaatgtaaataatgtaatgttcggctaaccttctccttctctccctcccttccccttttcttttcagctctgaagattcatttgaacctgcttaattacaaatccagtttctaatatgctatacaatttatgcacgcagaaagaaatagcaatgtacacatcaccttctttatatcttactttaaatattttatgcatgttttcaaaaaattggaaatatcctagatagctgagcaataaatcttcaataagtattttgatcagaataataaatataattttaagaacaatagttgatcatatgccaaaccctctgtacttctcattacttggatgcagttactcatctttggtctatcacaacataagtgacatactttccttttggtaaagcaaagaggcctaattgaagtctgtcactgtgcccaatgcttagcaattctcacccccaaccctgtggctacttctgcttttgttacttttactaaaaataaaaaac

SEQ ID NO: 17 - DNA, 1289 bp, synthetic construct
  misc_feature 1..1289: note = Description of sequence: Gene: HLA-DP
  source 1..1289: mol_type = other DNA
tccagcctaaggtgaacgtttccccctccaagaaggggcccctgcagcaccacaacctgcttgtctgccacgtgacagatttctacccaggcagcattcaagtccgatggttcctgaatggacaggaggaaacagctggggtcgtgtccaccaacctgatccgtaatggagactggaccttccagatcctggtgatgctggaaatgaccccccagcagggagacgtctacatctgccaagtggagcacaccagcctggacagtcctgtcaccgtggagtggagtgagtctctgatgaccrtctagaccccacctctgaagagcaggggactctctggctctggggtccactcatcttatcttctgcatctataccctggggccatgtccaaaccccatctttcttctataccagctcctgagcatagtttgaagccagggcaatggagacttcctgaccttggcttaggggttcctgaagattcatagttctcccccttgtcagagaatctagggacactggctgatctcgaaaccctcacacacaggaactgacctcacacataggaacagttctcttctttcagcattttagcctcttctcaggcattttgagaggcaacttctagaatcagcatttgccaccttgttgaggtcacacccctgttccggacatgagggtggctctttctgaatttcctcttagcaagctttttcccctgcactgtcttcatcccgatattctgcatcacgctccagaatctcagacaggacatgagtagggatgcagctggtggaggtgacactaaacctgggtctgtccttcccagaggcacagtctgattctgcccggagtaagacattgacgggagctgggggcttcgtgctggggctcatcatctgtggagtgggcatcttcatgcacaggaggagcaagaaaggtgagaaagcctgcaaggtgagcgggacttaccttcccctggcatattcacacttactccatgatgagggttcagacagaaaagaaatgtcagaaagctctagagaccactgaaatcagatagtcggggaacaaacatgacctatggcgagagggggatcccaggctgggatcttaatgcagccagatgcatgaggtcccaggtgctcagactcctgcggggcatccattgagtggtggtcaatggaatttggtgggatggaaatgtttctctaactatctgaggtggtttcaatggctgaatacataacctttcctctttcatttcagttcaacgaggatctgcataa

SEQ ID NO: 18 - DNA, 249 bp, synthetic construct
  misc_feature 1..249: note = Description of sequence: Gene: HLA-DP
  source 1..249: mol_type = other DNA
cttttccagggacggcaggaatgctacgcgtttaatgggacacagcgcttcctggagagatacatctacaaccgggaggagttcgtgcgcttcgacagcgacgtgggggagttccgggcggtgacggagctggggcggcctgaggcggagtactggaacagccagaaggacatcctggaggaggagcgggcagtgccggacaggatgtgcagacacaactacgagctgggcgggcccatgaccctgcag

SEQ ID NO: 19 - DNA, 283 bp, synthetic construct
  misc_feature 1..283: note = Description of sequence: Gene: HLA-DM
  source 1..283: mol_type = other DNA
ggtggcttcgtggcccatgtggaaagcacctgtctgttggatgatgctgggactccaaaggatttcacatactgcatcttcttcaacaaggatctgctgacctgctgggatccagaggagaataagatggccccttgcgaatttggggtgctgaatagcttggcgaatgtcctctcacagcacctcaaccaaaaagacaccctgatgcagcgcttgcgcaatgggcttcagaattgtgccacacacacccagcccttctggggatcactgaccaacaggacac

SEQ ID NO: 20 - DNA, 564 bp, synthetic construct
  misc_feature 1..564: note = Description of sequence: Gene: HLA-DM
  source 1..564: mol_type = other DNA
ctcctactccaatgtggccagatgacctgcaaaaccacacattcctgcacacagtgtactgccaggatgggagtcccagtgtgggactctctgaggcctacgacgaggaccagcttttcttcttcgacttttcccagaacactcgggtgcctcgcctgcccgaatttgctgactgggctcaggaacagggagatgctcctgccattttatttgacaaagagttctgcgagtggatgatccagcaaatagggccaaaacttgatgggaaaatcccggtgtccagagggtttcctatcgctgaagtgttcacgctgaagcccctggagtttggcaagcccaacactttggtctgttttgtcagtaatctcttcccacccatgctgacagtgaactggcacgatcattccatccctgtggaaggatttgggcctacttttgtctcagctgtcgatggactcagcttccaggccttttcttacttaaacttcacaccagaaccttctgacattttctcctgcattgtgactcacgaaattgaccgctacacagcaattgcctattggg

SEQ ID NO: 21 - DNA, 12431 bp, synthetic construct
  misc_feature 1..12431: note = Description of sequence: Gene: HLA-DOA
  source 1..12431: mol_type = other DNA
gggcattttatacttagagttttttttggcttaatagctacaggtggcttgtggctactgcacaggacagcacagtccttgaggaaggggatcatgtcctgctcttctgtgctcccagagcagcacacagtgcctaagtcacatcaggtgtcctataaacatgtatttaatgaagaatatcccccaacacctacctgaacttgtcatgctccaaagaacatgacagatacaactttacctcaaagtcagcattttagacaatttagaacaactggatttgtgtcatttttaaaggacactttagtaagtttttgtcgaatctgtgtttgcctcattattagactctagtcccaataacagtacctctccctagagtcagggttatccactttattcacttttatttgttatcattgacttatttatttaataacgtgctagcgtttagggggaaaaggaagattgtgtaacaaagattcaatcatccatgatcccttcttacaggccctttccatctaggagggaatgtggaccatacatatacattccaagaaaacaaataatgaggaagccctggtgagactgaaaggtgttctgaactgggttgcttgctggctgtggctatgagaaggttcctggaaccttctgctcttgcctccccggtgttggccttgagcaatcactgcctgctccctgctgcaaaaaacgatggcaagatcctgattgcctgtccagctgttgaggggtgggtgagaaaatccaagctgaaatcctgctggagtgggaggagctggggactgccctagtcattgcctcctctgccaccccagctatcagtgctccctacgcctcctagtgctcccaccctgaagtcctgacctgaagtgacacccaaagggtagtgactatggttttgcgctggcctaggactccagctggccctcctttctctttacacacatgacaccaacacagatggccactagtgggtcaaaataatacctttagggagtgccgacacctcaaaagaaaaacagcaaacaggaccatctatgtccttgaaagaagtggtagaaaatatagaccagccgcttaaaatgagcattaaaagtgcactttaaaagtcagaagctattataatatgaaaaaatagagaatatagggaaaacacctagaagttctaggtgtaaaaaatatatataatagtcaaagtaaaagccacattaatgggataaatagtgggagagatgcaactgaaaaacaaaacagaaagatggaggactacactgaggagctagttcagaacaaaagagggaagaattgagaagtcatcaatagaaaagtcaggggaaaaggaggagagaattatccaaatatagattatatgagtttcacaaagacaaaaaatagaggagaaaaatagttaaagaaataaaaagatgaattcccagaattaaaagatagagaagatccataatgtcttacagagagaaggaaaaacccactctagacacactagcaaaatttaaaaatatcaaagacaatgagaaaattctagaagattccagcatgaaagactgacatgggattttctaactgtgacattggatgcaaaaaaaaaaatttggaatgatgttatttagaatattaaaggggaaaatacttcaaatagtatgtaatacacagctaaactttattcaaaagtgctggtataattttaaaaaattatcaagcatacaagactttagaaggtttgctatgcaaattcccacatctgaaagtttttggaagaagaattctaatcagagaacgcattctagaaggtgccaaaagatatgaaaagtaatgttgaccaaataatttggtgaaggagaaaatatgtgaataaatttgaaagcatgctaaagttcttatccagtcagagggaagatggagaaggtatcggtaacacttaaaaaaaaaaaaaggcaatgaagattaaaaaagaaaacttggccgggtgtgatggctcacgcctgtaatcctagcagtttgtgaggccgagccaggcagattgcctgagctcaggaggataagcctgggcaacacggtgaaaccccgtctctactaaaatacaaaaaaaaaaaaaaaatagccaagcatggtggcatgcacctgtagtcccagccactcgggaagctgaggcaggagaatcgcttgaacccgggaagcagaggttgcagtgagccagcccagatcatgccactacactccagcctggcaacagagcgagactccgtctcaaaaacaaacaaacaaacaaacaaaaaaactttaaaaaatagaaatattgaaataaataaggtattacaaacaagcccaagtgcatcaatattcacaataaacacaatcagaatgcagcaaagacagtgccatgtgctcaccaaaccattatatcttcctgcacacataggtaacgtacatttttccatccccgtgcgcctatgtggggtcattgtttacttcttgccaatggaatgtgagcagagatggcaaatagtttctggtccaaaaccatggaaacaatgtgcctttcccaggttctctctcctacctgcaaagctaggagttcatgaagtgcttttcaataatagagatgcaaagtggagcttagccaccttgaggcatgttttggaaaagagctacagagtgcccaacctgccttgaactacacgtggctaaaaaataaacctgtgttttgttaaaccactgaggcttcagtgtttgtctcttgcagaaacaagtttgctcactttgactaccatatgaattgaacataggttagaagacagagatcgtcagattgaatcagaagcaaaaatctatatttatgacatttcaagggctacatctaaagcataagtacaaaggaagagtaaaaacaaaagagtagaaaaagatacagtgaacaaatacccaaagacagctgtagaacttattactatcagacaagacagagtaggacagaaagattctcaggacaaattgggaaaaggctcagttcaccaagactgtaataattcccaagagtaaacaccttaataacctaaatgcagagagggcaaaagttgatttaaaaattagaaacaagttgaaaaacctacagtcacagttagagatttaaatataccattcataggccaggaattgtggggcatgcctatagtcccagctactctgggaggctgaggcaggaggatcgcttaagcccagctgtttgaggctgcagcgagccatgattacttcactgccctccagcctgagcaacagagcaaggccctgtctctaaaaaataaataaataaatgcaccactcttagttatgtataggccagtccaaagataaagaaaaccgtaataaattagaatgacaaaatccacatgcttgatccaataaacatgtacatgtataaaattctgcatcacatttattagagcccatgtgaaatatttacagaagttcaccacactccaagccatagaggattatcatgtaattaaattataagttaataacaaaaaacatactttaaaaaatctatgtatgttttcaaataaaaaaaacacccgatactcgactgttattaaccaaagaagcaacattctttatcatgagtttttcatggctttcccgtctttcatctcatggctccagctcccacctcacttggggggaggtgaaggtggcaagggtcctaaccagggcaggaccaaagcagtgaagacactgttcattcatgcgcaggtgtctcctaggcgcctatgatgtgctaggcattacgtagacactgtggactagaaacgaaaaaactaagattagggctccccataaaccgggccatgcaaatcaattactagctgtcatttgattaatttgaagtcagaagctgtgtctgccccacttaaaataggcatcccttattcattgaggaaagagcattggagcaggttttaccagtccacagactacccgagaaccacctggggagcatgctgaaaacacaggttccctggccctgccttggaaaggctaatctaacccattaagtaaagaggaaactcaggaacacgtattataacgaaccccccaggtgattctcatttggaaccagagacccgcatgatttcctagctccctccaagttgaaaatcatcgatttcatggtctttgacacatggcctaatttggttagatttctctttacacagacgttagctttacgttgttgttgttttgttttgttttgttttttaaatatatatactggactaaattctcccagtgcctcttattagccgagcccaaccagaagccaaaggcaagggtgatgccgtcctccagcctgtgtcagcctggaggaggtgcccgggtcctggagggaccaacagagaggaccagcacctctgccctcctcggggagtccacgggagctctttacactttgctttagaccaaacaactccaacaattacacattttttcttacacaatgaaattaaataataataaaagaaaaataaaataacatttttgaaagtgatttaaagttttaagaggtttttattccattgtgtagccaatgttttccttttaaagattattggtaaagtttattttactttctaactcaaaatttgtccacggaaacttcttgggaaagtaggattccctgcatcgcagatggacatagagagaggatttgttggagctcagctgcgcggtgctctgacggccttttctctctcttcagggggtcccttcttggaggacgtggtaatagttggtccaagcccctcccatcctccaaaacttcctctggcctctcctgggggcccagtgaatccttccaccttctcaccccggctctttccctctttacccagcaacagatacattcactcagagaatttctgtgattggctgaagacagcaggggtcgcccccatcctcgaatctgttttcttcttctttacctccgccttgttcctgtcctcaccacacggactgagactgatttgattaaagcaccagagtgtaatggccctcagagcagggctggtcctggggttccacaccctgatgaccctcctgagcccgcaggaggcaggggccaccaagggtgagtgcgagggcgaggagggtgcggcggggagcagagatttacggagttgggttacatgaggaggtggcatggaggatgcttgttcctctcgctctctggtttatgggcaacttccttcaccaagagacacccaatccccctcatctctgtcacatccactctggacctaaatgaagatgcagctcggtcagctgcgcaggtgccccagtcagcctttgctgacgttcagatttctcctcattccttcctccttcctgagacccaaacctccacccaacagatgccagcaagcaccctgatttctctaccacccctggccgggaatgtgcccgatcaagtccagttctgttgcagtatttatgcccatgccggtagttaactatttacctgtctttgttcttcgggagacatgagctggggtgcgggtctacagatggttcatcttttttttcttttatttccctggccccccattgtgctgggtgcatgctagttcctcaataactgttgctcaaacaacttcatagagttctacaagaattaaaacttaatccctaacttccagaaaactagacaacagttatggaagagccacactcagtcataatgctctgagatggaggaattgggacatgaaccttgacttctgactcctcgtccagtgctctttgtaatgccttgagttgcctctccctatccccttggtctctgggtcactaaccttaattcttacccctgccagccgtggcctgtttcccctcacacccacctgcactgcatctctgtgcaaagctccatactctcctgtccttaatcattcctcctcatcccacccccacagtcccaccagctcacaacacagggcctgacaacacagccagggcagatgaccacagccaagatttaaattctggaaccccaagcatgattttaggcagtccctcctcttcccatcctgatatgccagactgcactgtctctgtgccgactgcagtgtgctgggatgagcctctttcttcctgttctctctccctttctctctcccagggccagcccagtgtcagaacaggactctgtccccacacagaacccaggacggggcccaggctcagggactcaacaatcacatattgtggatgagacagacacatttttttctctctccttgaccctgaactcgccaaacacagctgaccacatgggctcctacggacccgccttctaccagtcttacggcgcctcgggccagttcacccatgaatttgatgaggaacagctgttctctgtggacctgaagaaaagcgaggccgtgtggcgtctgcctgagtttggtgactttgcccgctttgacccgcagggcgggctggccggcatcgccgcaatcaaagcccatctggacatcctggtggagcgctccaaccgcagcagagccatcaacggtaccggccctccctctgcccacccagtcaggcgggaaggtccagagaaacttcctcccagttcctaggctcccatcactctggggcgcgctctcagcgcccgcgcctgtcatgccctgttcctttctttcccaggaggctccaggtcttcccagacccctttggcacccctctccttgaggaatgacacctctcacccggactcccgcccagggaccagtcaaatataggagctcctggcgtccccactccctccccagtctcctctccctctgttttcctcctctcctgccccagtggataccccagagcatcccctgcccacagatggctacaaagggggaacgtcccttaatcccagtcctagtaaggccctggggtgagggatgagcctgtggactcagggcctgttcctcctagtgcctccacgggtgaccgtgctccccaagtctcgggtggagctgggccagcccaacatcctcatctgcatcgtggacaacatcttcccccctgtgatcaatatcacctggctgcgcaacggccaaactgtcactgagggagtggcccagaccagcttctattcccagcctgaccatttgttccgcaagttccactacctgcccttcgtgccctcagccgaggacgtctatgactgccaggtggagcactggggcctggatgcgccactcctcaggcattggggtacggagccccctccccatgcaccctcctggccccaggtttcctttactctagaatcctttcatataccaccaactccttcctttctctcctagagctccaggtgcctattccaccaccagatgccatggagaccctggtctgtgccctgggcctggccatcggcctggtgggcttcctcgtgggcaccgtcctcatcatcatgggcacatatgtgtccagtgtccccaggtgcagaggccccgggagtctggggggtgggggaggaaagtggatgactctgaacaggacgtgggtggagaatcagagattctgttgtggggaaagaagtcagaaaagaaatgggcagggagaaaagaagcagaggtggggtgagagagtgaggttttgggggaggtgggcactcagagataggatcccagcatattgaaattgagcaacctcgatcgtatgttttctgctattttaggtaatgatccttctgagagaaatgacttgtgggagacaccctgcagatcctcatgggtttgtgacagcccctgcgtgctcagtgccctttaagtgcatcccgctgtgctgactttgagtgggatcaacatctgtcctacgggtcccctcttttttggccccagtattcatggcagggtttgttggacacctactagcttcccttcccattcaacacacacacacattcttgctctacccaaagctctggctggcagcactaaatgctttggtggtgtttgcactgtgtcctttccaggccttggccagttcttccaggggtgaggcatgtggtgctggggattggcagccatcctggggcccacacaggtgtgtcttgctccatttggcccattgtgtgttactttgtgaatgagccatttcacatggacttcatgaaatttgcctcctgagttcaggtttaccctgaaagggatgcagattatcctgttcctcacgaccccctcagctaacaacagttctgaagggtgctgggacaggacaggctcatggggactccactcctgcctgggtttactctgtatgaagaggccactggtatcctgccatgatgttatctcctttttctacttttccctagagtcccatgcatgataaagagaggcccaaggcttggataaggtggccacttccctcagtggagtcagtcatgttaggtaggaggtggtagagtcggtctgcgaggtatctcgtaagaggggaggtccacctagacacactctaaatatgtggcctagaagattttggtctacttttctgtgaacagaatttaaaacatacaaagagataaatcaccataccacatagtttatgtcaggaccaaaatgagcaatacagattacggttttcaaaccagaatgcacataagaactgcttgggatccttttaaaagtacaggcattggcctggtgcagtggctcattcctgtaatcccagcactttgggaggccaaggggacaggactgcttgaggccaagaggtggaaaccatcttgggctacatagagagaccccatctctacaaagaaagatttaaaaattaaccaggcatggtggctcgcacctgtattcccagccactggggaggctgaggccggaggagtgcttgagcccaggagttcaaggctgcagtgagccaagattgcgccactgcactccagcctaggtgacagagtgagaccctgtctctaaataaataaataaataaaatataaaaataacagtcatcacccagacctactgaattagaatctcgggagtgcagggggcagcaacagggaggctgtcttttctgagatggggtctcactctgtcaccaggctggagtgccatggcatgatctcagctcactgcaacctccacctcctgagttcaagccattctcctgcctcagcctcctgagtagctgggactacaggtgtgcgccactacactcagctaatttttgtattttaagtagagacggggtttcatcatgttggccaggatggcctccatctcttgacctcgtgatccacccaccttccctcccaaagtactggaattacaggcattagccactgtgcccagccgaggctgtcatttttaaccggctctggatgactctgatgcagccatcctggaccttggctgtggtctggtaactggaacccagtgacgtaatcaggtgccatcgggggtcatgggaaagggggatccccaaggtctgaggtggactaggaaggctttctgaagaacctgggtctgttagggcatcagccaatcaaggtacaagtaaatagaggcaaaatgagggtttgaactgtgagcagttggtcctggaaaagaaagaaaccaagagattatggggactcaatgggcttcttaagagagaataagttgaaatcaatgaccagaagaccctgatggaagtggaggagaatcatctcaggcaaactttttgtgtgccagtaacagaaaccctctttgtgtgatcacatgcaaagtataggatatttgcaatatagccatggggaggagtgcagggcccaagggtagattttagccaggcctcccaggaacagaactcggatccgaaaagcccagagaagctagagctgcccctccaacactctcggatccacatggtctgtgttctctagacccccctgcatgttagcggtgttctctctctgtggactgactgtccttctcagtgaacatgtccacccgacagctcctgagtttatatcatctcaaccctcacaacccacagaggctgtgtctcctagtcacagctttaaattactggaaaaataaatgactggccaaacttggagcaggtgtccatcccagccctgtgtagttagagcaggaatcaagatctcaacacaaatgtggctgccaagcactcagccccggggcgaggggtcaagttcttctcagagaaagaggaataagttggttctcagaagacatcacaagatacgtgtgtacccaacaatctctgatctctgctgatcttttgcttagacgttaacttgatgcatcattggaaaggtgtttctctcatctctgtcctaaggcttgataaagtcattaaaattgtgttcttttgactaaagaaatatgctttttttttactgttgcatatactaccctgaagtcactggaacttctaggagtaattccagagcttttagatttatgcacctgcgtgtatactcacatttgtttctagtctcaaggtacgtagtcttttattttaaaaaacaagttgtcctcctgcattcttgatctttcactctcttttagaacagacatccagtagtctcctgtcctttgtatccaaactctccctttttgcagggtcaccctacaaatcataagcatgtcatctctcctatataacaagaacatcaaatcctctgtctgtccttgacccagtctcccttccacccaagctcctggtcacgctctctgagagtgtctacgtggactgcctccagatcccctcttcccatccactctctttcggttttaatttttaaccaaattatgttatagtttaaagagtcaaatatttctacaatatttgctacagaaataccagtttccagccccatctcccaccatatcctcacccttacagaaaataactttcaattgttctaatatgttttgttggtattaacctcccatctctaaataacatgtttgtgttgctacatctagatttttcagctttaggctttatctctttacctcctgctgtggaagagggggatttagatttttttcatcctcaaagaacatcatgccccctttcccatgccctttcttttaatgtgactatattgtaatttttacaatatagtaaaagttactgtgtttactatgtttctatcaatatgaccatgtaatggcaaaccatagagcaagccatgctcactcttccttttctagacgacttcgttttctctggagtatatagttgtcctgttttcctttgtgtggctgtatttgtacttattattaatgaatcacaaaattcctcacagtacaatcaaacacatcaggtattctgtcaattttatcaccagatataactctcccagaaccttctgtctgctccattcttaactccttgcccttgatgatacagcggtcactctgagatcactcttcgccatcctcgtcaggatttacctatcctccgagttggatcttctgcttcttgtatccagtcattgcccttttgtggtctgccttattttaatgaggcacctcttctttccacttcctgagaaacagtgcatatgaggaaaactttttatgcttcgtatgtcggaaaatatatttattctactctcacacttgaataatagcttccacggttataaaattcaagattggaaatcatttccatcagaatttttaaggcatttctctataatcccttagcttcctgcgttattgttgataatccaaaagatattgtgtcatgaaactttgtacgtaatcgttgttgtttttttctctcagaagcttataggatatctgctttttcccttcctcctttaaattcttataggtgctccattacctacaagatgaagacccttctcaaaccgtcctcagggtatttctttttctttttttctttccttttttttttttttttgaggtagagtctcactctgttacccaagctggagtgcagtggtgcaatctcagctcactgcaacctctgtctcccaggttcaagcaattctcctgcctcaccctcctgagtagctggaattacaggcaccccccaccatgcccggctaattttattatttttagtagagaccgggtttcaccatgttggccaggctggtctcgaacttctgacctcaggtgctccgcctgccttggcctcccaaagtgctgggattacaggcatgagccaccgcacctggcctattttaaccatataatagctcctcctgaaaatgatcaatgcttaagtcacagaaaactgttgtcgcatgtttactcctccctgcctttgcacaaactattctctgtgctggacctgccttcctccaccaaccagtcaaaattctacctgtccttcca

SEQ ID NO: 22 - DNA, 11286 bp, synthetic construct
  misc_feature 1..11286: note = Description of sequence: Gene: HLA-DOB
  source 1..11286: mol_type = other DNA
cactaggtgggatcatgagtttgctttggacaccccaaattctaactatttcttttgtttcttacatcctttccctcttccccagccccttcccctcatgttacacctcttgctggtttgagacgtcaatcaccactgagaaagaattaaaccagtattttgagctggcaaaattcttagcctagtacaattccttcaattaaactgtagctcaacaatgtgttctctaaaagtatgatttaatattctacctagaaactcagaatatttttcataactcctccagggcagtgtggattatgttgatgtgttaggaggaagtagaaggaagaaaatgaattgagatgtacgttttacttccatgtcaaagtcaccaaaatagcagttgaagggatatttgtgtcaggcaatagggagaataataattttatgccaccctcaaaaaacaaccacacatacacccaaattctctcactcctcagagaaaggggaaggaaagaaaagagagaagcaaaatatgagcttgagtgaaaaatcacagaggagcctgtagctatttagggagggctgctggctgaagtcaggcaggaaaaaaagatcaaaacaccctgccctatttttcaggctcgttcaagtagaagacaaaaacataaatacaggagaaaggaagaaaaccacatctttttcctcttgtctccccagaactgaacagtgtctctgataagcccaggcccttctatcgtagacactgacactatgcacagaaaatgactcaaaaacgcttctaatgggggtgaatctgatgcttcagtttatttaagatgtaccagaggccatctaaggagatccatagcttgctaactgaagcttattgctttctcttcttagttcccatgccaggctctatgccattccttgcttgtcatgtgagatcactgatttcctttggttaggtaggatatgatttccttacagagatctgtcctgacaccaaacagctatgatgaaattccttggttattttccctttttgtgtaccttatcattaccggaatgctaaagctgtaggaataaagtttccctggcttcccaagaaatacagtgtgacagaaaaagtatagcctagggacttactagttatgtgaactttggttcattccatccttacctcatctgtaaaagggagatcatgatagtatctactgcaaagggttttgtgtgatgattctctctctttctctctctctcatacacacacacgcacacatttatataatgcttagaaaatgaactcataataagcaattgacaaacattagctattattatgtaggcaagtcagattttagagtttgtgagccttagacacatttacagagaagaaagagcagccctcctaactttctggtccagcgccatatcctccacttcctccccatccccacatcccttgccatttatcaaccccctctctcctctaaatctaaatacaggcccccgtttgatccatattgtctaggcccttcctctcctctccacatggcccttccttcagctctgagggaagctgcagaagccagccatggtgctgtctacaaagaaggggacacacgcctcttccacccgctcatgctgttactgcatctgatcatctccgtgccgtgtccttgttcacttagcctgtgttgagtgtttgtctccatttccaaatgcaataaaacatctgggaaagactaaggtaggtgtgggcaggaagaagggaggaagttagacccagtggcttgagtgccctctgatgcctccttatcctcggctccacacaagccctcgccagtgtgagctccacagccatccacctggaggaggagtactcaaaaccagggtcaaatgccttgtactcgggggtctaccagtaagcctgtggccagccttccacttcatgaatcgtcacatttacatgagagatgtggagggagaggggtcagcctcctagctcctgtcctgtagcagttaagtcaagtcaggccaggctgtggtaaccaagccagtaccttctttaaagaacagttgtttctcattcatacagagtgtgttgtaggtgtgagtgattccctggggcagctgctcttcatggtgactcagctaattagactgcttttctactgggattgtgccatctcaacacaaggctgtcttggttgacttggtaggggaggaggagaagagtcatgcacgaggagttaaattctttgaccaggaaccttttgttcactttccattggtgagaacgagtcacacagctccaggagagtaggaagtctagtttctcgttttccatgggcctaggaattagaggatgaacactagcgatgtcttccacgtcttgctctctttttctttacctgttataatctcccatggagaagaaattatttttctgattggtctgatccaggcctctaaccaagggcatttactaagggtcatagaacagtaaaaggttgcacaattgcatctcacctctcttttctgaatcctgcttaattaaaactttaaaaactcacttacagaccacttcttgctcttcttagttatcaaatatattttcttccaactcctttttgacagatcttgccttgttctcagctgactcccttgcaaccatctaaatggaacttcctcattcccctttatcccctgctaatcgtcctgtctttattcttttatctgaagaagtgtacccttcttcccccaagccttatgctctctgtcttagtccatttgtgttagtgtaacaagatacacgagatagggtaatttatgaagaacagaaatttatttctcacagttctggaagctagaagttcaaggtcatggccctggaagatttgatgccttggccatcgacctgaaaaaagtcactgaagtgaacttgtggttaccatgataccctgggttcattcttaacatgcacagtataggcaaatcaggggcacatttcatgctgaaagatgatgtgaaatgatgagtaaacattgccctttaaataataataacatagatggtgaattacaatttgcacttttggttttatttgcttaaagatagtgttttactgtctaggttttactgaaagaatacttaactaaacacttgagtcaaatcattccagtgtaacagtcctcacaaggaggaaggcagaagggcaaaagtgccaagctattgcctccagcccttctatttttattgattgactgattgattgattgagacagggcctcgttctgttacccatgctggagtacagtggtgcattcatggttcactgcaactttgaacttctgggctcaagtagtcttcccacctcagcctccaaagtaactaagaccacaggtgcatgccaccatgtttatttctttaattttttaaattttatttggacatgggggtctcactatgttttccaggcaggtctctaactcctagcctcaagcaatcttcctcctcagcctcccaaagtgctgggattacaggcatgagccatcatccctgctctctctagccctttttaaagtctctaatcccattcaggaggcctctgcattcatgacttaatcacctcctaaagggcccaccttttgatactatcacattggtgattaaattgcaacacttgaattttgagtgacattcagaccacagcattttggagaaaatgtcccctttcttctcttttttctcttcagtttctcctttctacagataccttgcacttcacttgcaaccttgttgaagtctctcctgtcttaaaagtttcttttcccaaaacaccagaactcattccttctgtctaactgtaactttgtacttgttaccaacctctacccatttctacccctgactccccagcctctggtaactcctaacgtactctttacttcgatgaggtcaacttttttagattccacatatgagtgaaatcacgtaatatagtccttctgttcttggcttatttcacttaacatagagccctccaggttcacccatgttgttgaaaatgacaggatttcatgcttttttatggcggaatagtattccatcatgtatatatatcacatactaattaccctgatttgatcaatacacaatgtataggtgtgttggaacatcatgctgtaccccataaatatgtacaattattacgtgtcaatttaaaaaacccaagaaaagtattttttcttagtgttgtttttttctaaagtgtatcccattttttcctttgttttcactttcaaaattcttgaaagactgttttaatctgcggcctttgttttcttgtttccaattcattcttttgaaatatggcttctacctttaacactctgctgtcctcttgaaggttattggtgccctactaaccataaaacacaataatgctctcttagattgtatcctggtgagcctttttgcaaaattttattctttgagaattctctttccatgttttcttttcttttctttttttgtggtcattttggcttataggaaatcgtactctccaaatgttctctatgtcttctatttcttctctgataatccctttactaaatcttcttgaggcatatgcttcagaatgcttaatttacatactcttaattccttcttaatttacacactgcctgtcggcaatgtcaacacccaataagaagggagacctattggtcagaacaggccagggaatagaagaatacataataaacagtctgcctagttcttctagggccccataatatgtcaaacatatatttttacttcttctcccagcccatttttagtatacctaaattactgtcagtgattctgcaggcaaacaatggttgagttgtatgacacaactttgtgaaagtatcctacccagtacctgatgatgcaaaactcttctatcttgattggttgtcaatctgaggagtttccaatcctggggaagccagaaaaacagcgatttatactcttaatgggtactttctgactgaattttatgagctcattctgaagaggctgacgattttactatctcatttttttcctttctccagaatgggttctgggtgggtcccctgggtggtggctctgctagtgaatctgacccgactggattcctccatgactcaaggcacagactctccaggtaagaacagagcaattgtttttttccagtgtgtatgcaagaattggcatgggggagtgatgcctttctttgtaagtccaggccacagaccagactggaagtggcttttggtttcaaagaacagtgttcttccctttggcagaaaggtacgccttgcctctttacatgggatggacttcatataccagagccacctattcaaggggtagggaggcaggaagagggaaacattgtgtcttgtttaggatccttattgtgtgtatcaacctcagtcagtgcctgggcgtgttgaaggccttggcttgggttcgagcctgctgggagaaacaacctgcagtaggctgggtcacagaggcaatctgtgattttttggtcaggacacggaaacaaatctcagttggggtatatgtggacaaatgaaactggaaacaaaggttgctccttctgtcatttattaagccactattatattgtcagaattgtactaaacagttttgagaagtaagagaagttgaatagaatacattgtccttgtcctccggctaccaggtacaagttacttgtcactgttatttttctagcacaggtgacagaatatgcagccatgaagcaatgtgagatgaaagcacatattaatgagcagaaacaggatgtaatgtgctaagaacagaatcccctttgcatgttagtttcattaaatacaaaagaggaacaaacctggccaggagagatcattattctcagagaatagaaaccgccctgagtttataatgtccattaaacaatacaactgaaaaaaaaatcagcacagatgttaaatgatgatgaaaaattcagatttcccccctggtttagactactagaggaaatagagaagagtatacatgctgagaaattacaggctggaacttcatctgaaattagctactgagtgagggataagtggggttcacccaggaaggtcattcttatggctcagttcagagttggaggaggcttctgaacttagaaaggaaagtaaattacaacccaacattaatagcaattatctttcaagtcttgacttagatgcaatgtcttcaggacgtccttcctgacttacctacattattaactccatttgaatttcctttttattgtagttgttgttcttaagtgcataggattggtttaattttacccaatgagttcacagcacattgtaattattggcagtagtgcaagactctctcgtctcttctcttgcctccgttctcattctctcccctccctagagaatccattctaaacgtgtctggtaagtctctaagtatgagagtggcttttagaaatatgtagcattattgctctttatgtgtttttaaaatttaataaatgtcattctctgttgaatcccattctgtttcttttctcacttgacactgtgctttttgagcatactgaggtcaaaggcctcctcttagatccatgtggtctgacgtaatttaccaggcatgggttttcccagaggagggggctggttcatggttttggttttggttttccagaagattttgtgattcaggcaaaggctgactgttacttcaccaacgggacagaaaaggtgcagtttgtggtcagattcatctttaacttggaggagtatgtacgtttcgacagtgatgtggggatgtttgtggcattgaccaagctggggcagccagatgctgagcagtggaacagccggctggatctcttggagaggagcagacaggccgtggatggggtctgtagacacaactacaggctgggcgcacccttcactgtggggagaaaaggtgagctggaagctgaggtctggcggggctcaggaatgtcccccatgtgaacctggccatggctcttctttcttacaagcaattttctgctttaggataaatggttgtctgtgtagatgttctggccccagctgtgatatattatcctcacaagtcagccactgtgatcttggtctcagacccccaaggttctcagggacttcgagggctattgtgccctcaaagagaagcagtaattgtgggagtacctcagaaagtctaaatcctcctgacaggcattgacataccctgttactgatcttgggggctgagacttgcctatactttgtgttcacttgggtgatctgggaaagagattagacatagtgatagtccctaaagaatctcctgtcccagcttggtggttttctttcacggtgtctcatttttcctcccttcctagtgcaaccagaggtgacagtgtacccagagaggaccccactcctgcaccagcataatctgctgcactgctctgtgacaggcttctatccaggggatatcaagatcaagtggttcctgaatgggcaggaggagagagctggggtcatgtccactggccctatcaggaatggagactggacctttcagactgtggtgatgctagaaatgactcctgaacttggacatgtctacacctgccttgtcgatcactccagcctgctgagccctgtttctgtggagtggagtgagaattagtttctagtactctctgggcctgactcaggactatactgactcaatacagagcctgtgtcacttctgcgtttatcttggtcacaacatgaattattctttcccttgatctgggacagtcacagaaaccagagtccttgggttagggtgggagaaaacatggcagatatctatcctcatatcttccaagaaatgaggagatctaatcacctcattatgtgcttccaaccctatgaactggtgtcctctaattctttggtcttagtatttaggaggcattcttatgggctgtgagaatctgtaaccgatggggggtaactccatgggtgccaactttggtttcgaagaaccttttctaaatttatttatttttctctagctagcattggatttggtgtctagtacagattctgggattccaagaaagtgctttaaatattgggatatttttactaatttaaagacctgtttcccataggagctcagtctgaatattcttggagaaagatgctgagtggcattgcagccttcctacttgggctaatcttccttctggtgggaatcgtcatccagctaagggctcagaaaggtaatgagcctgtgaggagtgccctgccacctgtcccagaccttccccactcccaccttccctaacgtcaatgatctgaggcaaggaaagctgattgtgcctctcagggatcaccgggataatttttttctgaagctagaaatgggataagcagagagagtgctgaccttgccagccatttgttcttccctcgggataatcatattgggtcctaattggggcaatccattcttttctcgatttctttccaggatatgtgaggacgcagatgtctggtaatgaggtaatgtctctttttccttgtctttgagtggcagatcattctcccggttctttggccagagggagatgacatgggggtagggaggagtaaggttgctgctgtctggatgggactgtcccctgagtctctggaacggctgtggggggtggtgaggctgcctcctgagaccttcatcactgtgcctccaggtctcaagagctgttctgctccctcagtcatgctaaggtcctcactgaagcttctctctctggagcctgaagtagtgatgagtagtctgggccctgggtgaggtaaaggacattcatgaggtcaatgttctgggaataactctcttccctgatccttggaggagcccgaactgattctggagctctgtgttctgagatcatgcatctcccacccatctgcccttctcccttctacgtgtacatcattaatccccattgccaagggcattgtccagaaactcccctgagaccttactccttccagccccaaatcatttacttttctgtggtccagccctactcctataagtcatgatctccaaagctttctgtcttccaactgcagtctccacagtcttcagaagacaaatgctcaggtagtcactgtttccttttcactgtttttaaaaaccttttattgtcaaataaaatggagatacaaaaaatgtacattttagtgaattatttaagaaaaacccctgtaatcaagtcaaggaacaggactttgccagctccagcggaagtctctgtacgtcaggccaatcaaagcctctccttcccctcgaaagtgaccatatcctgattttattgtaacctctttcatgtctttgtagtctagtcccccaggtatgtgttcctggacgccacagcttagttgtcttttacgtctctcatacttcactggtttctcccccatcagcctttttttcccccttatgattaatctgttgaaaagctcaatccatttgacctgtagagtttcccacactctggattttgctggctgcgtacacatggtgcagtgtaacacatcctctgccttctgtgcctcccgcaaattggcatctggatgcagaggctggatcagactctggttcaatcttttcctttggaaaatctatacgtggtgttgtggcctttcaccatgagagacataatttccagctgtttctttttgtgatgttagcaaccattgatactaattgcttaggtctgttaatttattggggattgctaaatggtggtattctgtcattttttcttcatttatatgctttattaatgctacaaagagacagttcccttcatttactagtgaagagacacttccctccacttactagtgggctacttagtggtacagtggaaaggcaggatcagtgtttgactctccttttatttaccagttttcaagttagcaaattggtttcctactattaataaaacctacagatggccaatcaggttttacttttcattctttaaataattataaacagaaggatttaaatgtttgataggttttactctattgcaatttttatgcttgttaaagcttatatcctacatttttggtgagtgaaaacttcttcaagtggctaaaaatgaaactgagttcttttgacgcaaatttcttttgataatttctttgttatctgggatgacaacatgtttctggttcatcttgttcatttcttggccagacctggaggtagccatttttccagaaatccctggtttagtttagtgggaaatgacatttaagactataatccagcgttagggtgttttgtcccactgaacccgctgctggagtcacctcaaaagtggcgtcaagggagttaccacaaggaagttcctgaaaccattctgagatggtgtgggttaggattcaaagaaagaagcactaaatgccagggtgatcagtccaaaacatttgtttagggaatttacctacagagggcttcaggagtccttgcagacagcaagagaaaagggatgttctgcctaggtatgtctgcagcgagggggtcagggtatggagcttatatgaaggtttaaggaacctggctcagggctgggacaaagtttcagtgtttagagaaacaacctagatacgtttatcagtgcctgggagtgttcaaggccttggcttgggttcgagcctgctgggaaaaacctgcagctggctgggtcacagaggcattctgtgatttttcggtcaggacatggaaagaaagcagggagaggggcagtgggggagcctaaatagataggaatggtcattgtgtccagcctgttcaatggcaataaaacatttatacatgtatacatttaaaaaaatcagtcatccaggcacggtggctcacgcctgtaatcccagcactttgagaggccgaggcgggcgaatcacgaggtcaagagattgagaccatcctgggcaacacggtgaaaccccgtctctactaaaaatacaaaaattagctgggtgtggtggcacgcgcctgtagtcccagctactcgggaggctgaggcaagagaagcgcttgaacttgggaggtggaggttacagtgagccaagattgtaccactgcactgcggcctggtgacagagcaatactccatcttaaaaaaaaatcagtcttctctatattacttctgcatctcct

SEQ ID NO: 23 - DNA, 897 bp, synthetic construct
  misc_feature 1..897: note = Description of sequence: Gene: HLA-DQA1
  source 1..897: mol_type = other DNA
gttcgttgcaacaaattgatgagcaatgcttttttataatgccaactttgtacaaaaaagttggcaccatgatcctaaacaaagctctgatgctggggacccttgccctgaccaccgtgatgagcccctgtggaggtgaagacattgtggctgaccacgtcgcctcttatggtgtaaacttgtaccagtcttacggtccctctggccagtacacccatgaatttgatggagatgagcagttctacgtggacctggggaggaaggagactgtctggtgtttgcctgttctcagacaatttagatttgacccgcaatttgcactgacaaacatcgctgtcctaaaacataacttgaacatcctgattaaacgctccaactctactgctgctaccaatgaggttcctgaggtcacagtgttttccaagtctcccgtgacgctgggtcagcccaacaccctcatctgtcttgtggacaacatctttcctcctgtggtcaacatcacatggctgagcaatgggcactcagtcacagaaggtgtttctgagaccagcttcctctccaagagtgatcattccttcttcaagatcagttacctcaccttcctcccttctgctgatgagatttatgactgcaaggtggagcactggggcctggacgagcctcttctgaaacactgggagcctgagattccagcccctatgtcagagctcacagagactgtggtctgcgccctgggattgtctgtgggcctcgtgggcattgtggtgggcactgtcttcatcatccgaggcctgcgttcagttggtgcttccagacaccaagggcccttgttgccaactttcttgtacaaagttggcattataagaaagcattgcttatcaatttgttgcaacgaac

SEQ ID NO: 24 - DNA, 5639 bp, synthetic construct
  misc_feature 1..5639: note = Description of sequence: Gene: HLA-DQA1
  source 1..5639: mol_type = other DNA
gacatgcacacaccagagaagattccaatttagtgtcttccctctcttcatagaacaattcctcaagtccactctgagtagaggctgcatcacaacaaggggattgccctgtctccttccagggctcttaatagaaactcttcaactagtaactgagatgtcaccatgggggatttttctaattggccaaaacctgacttggcagggtttggtttgggtgtcttcagatttccttgtcttgaggtcctcacaattactctacagctcagaacagcaactgctgaggctgccttgggaagaagatgatcctaaacaaagctctgatgctgggggccctcgccctgaccaccgtgatgagcccttgtggaggtgaagacattgtgggtgagtgcgtgagtgaggaatgttctctggagctgaaaaacagtaaattgaaggaaaagagagaaagcgatttgcagagaaattgtagagatttcctaagacccctttcagtattaagagaattaaaaattatagctgttcctccttcaggaaaccagagccccaacctactctttttgttatgtatgcttttgtgttcactaaggatgctattctgtttatattatattcagtgactacagcctggaggtctctatgtcattccatcatgattgcctcaaaaattagtgaggtttccatcagtggataattttttattattaaaaatgtatgaagtgtcattctcaaatttccctgaacaacttttgaagattttcggatgtctcctgtagtagatcttggggtcgttccatcaattatatactctatagatattaaaaaagttgcccgtttctttctctcagacttactcacatttccacatgggaactggcacaggtggggagtaggtaaaggagtccagcaggctgaatgccttcaacaatcattttaccacatggtcctcacttactctcagctgcctcatatgtgtcacctcacaaataatcaaataaaatgggcatgtagctaagctttgtaaatagtgaaaacatggatgtcaattgtttttacatatttctattacaggtatagcttcacatttcttttctttagcaaaataagggatccttttagtttaaaattgagaagtagaaaaaattggtaaattaaatcattttattctcaaattatcaacccaaattacctgttcttcacctcatctaataaagtcctataaaaagaaaagtgggccagacatggtggctcatgcctgtaatcccagcactttgggaggccgaagcaggaggatcatttgagcctgggagtttgagaccagcctgggcaacacagcaagacctcatctctaccaaaaaataaaataaaaattagccaggcatggtagtgcatgcctgtggtgccagctactcagaaggctgcagtgggaggagcacttgagtccaggaggtggaagctgcagtgagccatgatggcaccactacactccagccagggcaacagagagagaccctgtctcaaaaagaaagcggaaagaaagagagaaaggaaggaaagaaggaaagaaggaagcaaggaaggagaaagggaagggaagaaagaagaaagaaagaaagaaaacagaaggaaggaagcacagattaattatttggtctcttagtctcctctgcctttgtcgtccatctcttcccacctctcttcatgcattcctttctccctcttccctttcaggatccatctctgactccctgctcctttatagagatggacatgagtttgtaaaacaaaagttgaaaagtcagatagttaaaaggggaagtaaactggaaggtactctaaactttcacaaccttattaaccgtggcagctcccattctgattttgttcagcagtggaagtttcaccctctcctccagagcgcttggcttctttgttccaaatttcctttcttcagcctcacaccagagtgccctggtcaggctcagctcatccattaggcacaatgtgggcagtgcaggggaacctccatactgtaaagccacatgagaatgttttaactccttttaaaattataaaaaaatgaaattgtagagcctaagaaaatgttttaacttttaattcagcctatattatattgtctttataccaattcagtcataaaatataattttccatatttttatggaggaaggcgtccacacaagcaagagtgcttggggctcacatgtcagaacgcatccctgatcatggctgatcctgaccttcgtgtggttctgctaactatgtgcctgtcagtcttccccaaaatctatgtggtcctcaaatataacaactgtcattcaatacacatgtttgagcacccagtgagctaagttttaaggattcaaagatgaaaagtcatgctgtctcccctgcagagggtgctcagactagtgatggaaacagtatgggatgaaagaaagcagaaggccattgctgagcaggcagtggactcagcagaggctgaaactatacaagtgacttggttccagctgggccagcaggataaccagacgaaaagaaggattgcatatattccatatatatttatgtttgaacaaagagtcaaggtttattgcaaggataaggaggctttgttggtggcctgttaagaccatccagcgtggtcatactggatagggaagaaggtgagctggaagagggatagacaaacttggatggccagatgttgagatggaggagctggaggtcataacgtggtcaaaaacatgttgatgagaggacttagctacaaagttgttaacttaagcagaaacctcaaggattgattttatgatttctccaggaagtcctaaaagataatttcatttcagggaggaaaacaacagaccactgcaaagaccaggaacatgaaaggataatgtagtttggtttgcttggcagatacttgtgaaagatgttggactgtaaggctgtcaatatcctcctcgcagaacttactacagtacattgtatctgctcccttacctacctgactctcccactattcagtttgttccttaatggtagaccatgcctgattggtgttttacacttcccctgctatgtctgatacttgtggatgctcagaaagtggggaaggaaggaaagatacgatggtaaaaggcttacacatgtcttgaccagaatgttcagtttggctcatttggctggagtcatactgcatggctgccattctgctctggcatcctcagagaagcacactgcccattagaggaaaaagggtgaatataaatgttgagtcagaacactgcagacatttagtaacctccttcagaggaaaaaaagggtggggggaatgacagaaatccaaaaactagtagagcttccactttttcatttcagaagaaatcagttactctcctctaaggaccattactattaacaaaacagagaccttagaaggaagcattatttatttatcatatattttgtaatgttattaccgttcttgttatactctttcttataccctaccattgttagcagaaattattttaaattaataagatcctgcatgcttttcctttttctaaaaaaagaaagatctctgtgtagaatgtcctgttctgagccagtcctgagaggaaaggaagtataatcaatttgttattaactgatgaaagaattaagtgaaagataaaccttaggaagcagagggaagttaatctatgactaagaaagttaagtactctgataactcattcattccttcttctgttcatttacattatttaatcacaagtccatgatgtgccaggcactcaggaaatagtgaaaatcggacacgcgatattctgcccttgtgtagcacacactgtagtgggaaagaaagtgcacttttaactggacaactatcaacacgaagaggggaggaagcaggggctggaaatgtccacagactttgccaaagacaaagcccataatatttgaaagtcagtttcttccatcattttgtgtattaaggttttttattctcctgttctctgccttcctgcttgtcatcttcactcatcagctgaccacgttgcctcttacggtgtaaacttgtaccagtcttacggtccctctggccagttcacccatgaatttgatggagacgaggagttctatgtggacctggagaggaaggagactgtctggaagttgcctctgttccacagacttagatttgacccgcaatttgcactgacaaacatcgctgtgctaaaacataacttgaacatcctgattaaacgctccaactctaccgctgctaccaatggtatgtgtccaccattctgcctttctttactgatctatccctttataccaagtttcattattttctttccaagaggtccccagatcttctcatggcaattgctgaaattttatcatttctcatctctaaaatcacatatccccatgtaatacaagggtctttccattatgcattcattaaatcattctaggagatgtctcatcaacctcctactttattaaacatgcccacagagagaagggcacaggagtaaagcagaggcaatgtgtcattgctcccaagtagaaggtaaataaggcctctttgaccagcaggagaggaaatgctggtaggaagactcttccaggatgtaatgcagaagctcagggcagagctattcacacttcacaccagtgctgtttcctcaccatagaggttcctgaggtcacagtgttttccaagtctcccgtgacactgggtcagcccaacaccctcatctgtcttgtggacaacatctttcctcctgtggtcaacatcacctggctgagcaatgggcactcagtcacagaaggtgtttctgagaccagcttcctctccaagagtgatcattccttcttcaagatcagttacctcaccttcctcccttctgctgatgagatttatgactgcaaggtggagcactggggcctggatgagcctcttctgaaacactggggtaaggatgagtttcaccattttttgatgctttcttgtctgtcaagttcagaacttcctgccttttactctatgtcccaaaacttgttttccacacttcatgagtttcttttatctttttttttttttgaaagaattaagcaacaaaagcacagatttattaaaaaagaaagtacactccacagggtgggagcaggcctgccacttcatgggtttctaataacagacttcactctcctccctgagccaggggccttgagtctttgcagagccaaccctccaccccatcccatccctcacacatgcacatgagcacactctgcattctgacctcaacaacttcacttccacagagcctgagattccagcacctatgtcagagctcacagagactgtggtctgtgccctggggttgtctgtgggcctcgtgggcattgtggtggggaccgtcttgatcatccgaggcctgcgttcagttggtgcttccagacaccaagggcccttgtgaatcccatcctgaaaaagaaggtaagtttgagatttgttagagctgaagctgcaggaaggaaagtgggaggaggctgtggacatgaatgtggttgaaagttgtaggggaattggg

SEQ ID NO: 25 - DNA, 5081 bp, synthetic construct
  misc_feature 1..5081: note = Description of sequence: Gene: HLA-DQB1
  source 1..5081: mol_type = other DNA
cacgggccggcgggaactggaggtcgcgcgggcggttccacagctccgggccgggtcagggcggcggctgcggggtggccgggctggggccgggccggggccggactgaccggccggtgattccccgcagaggatttcgtgtaccagtttaaggccatgtgctacttcaccaacgggacggagcgcgtgcgttatgtgaccagatacatctataaccgagaggagtacgcacgcttcgacagcgacgtggaggtgtaccgggcggtgacgccgctggggccgcctgacgccgagtactggaacagccagaaggaagtcctggagaggacccgggcggagttggacacggtgtgcagacacaactaccagttggagctccgcacgaccttgcagcggcgaggtgagcgtcgtcgccccgctgcgaggcccactcttggccggggccccgagtctctgcgctaggagggacgaggggggcgaagcctctggaacctgagccgcgttcgttccaccccaggggacaggagttggcggcgtgggtggtggggcaggtgcataggaggggcagggacctaggggcagagcaggggtacaggtagagttggtcaaactgcctagttttgccccagccttcccgtccgtcagcctcgccctctgctgtgcacgttctcgcctcgtgccttatgcgtttgcctcctcgtgccttgcctttgctaagcagtcctctctgcccccaatttctgccctcttcccctgcccgcccgccccgctagcactgccccacccagcaaggtccacgtgcactgctcgcgccgcaggaagcttcaggcttggcctggtggagttagggctgctccacaactgtgcgcagggcatccagcaattacagttgtgaaataagatattttgacttttggcttcaaattattattcatcgtaattccgttttcttaaatggctctcattcatggcggagctctttgaggtgagagtgttttaatcattttatggccggtacctgacacattgactggcatgtggtataagctcaataatcttctgttaaattaatgaataaatgtgctcagctgccaatccacttaggctcaagaaaaaccagaggtaaacagaaccttaaaaatggacttttattaattattttctatcattttgcttaattctttaaagtaaactcttattgacttggatctaaatagtttgtgaatacaaagtctgaggaaaaaagtgtttgctaaaaataaaaacaacacttgaatgatgtttgtaaggcagttttaatttcttagaaaagctgaacaaatggcacaatgcaaagagcagaagttttggaataaatagattgaagccattaaattattggataaaaatagtttcaggttgcttttggcctaggttctcccctccccccatcactatccacttcaggaataaacattctgaaagttaattttacccatatagtgagcacttatttctaagttgccttatcaaataccatctatgttatgtcatttaatctcgcagttgcctgtgcattagagattagcatcaccactttatatatcctaatattagtacatgataaacactttaagtaatcagcccacaagtactcaccaagaccttaagcctcccaaagtacacaacattcttacgttcctcactacacatctgtagagtcaaagggacataaagccttgttaaagccagttttgaccagaagcagcaatggtctcttcctgtttgatctccatgttaatgggacaaaatgatactttcaaggcattgaaaatttatgattaatcaatcaaatttatgattaatcaatccctagtctgactccagtgagattcacaaaactttcagtttactttatactcccttgccttcttttgactcacatcatagtgccggcaagtacttatatttttgctatttcagttctatttccataaaatttattttatcatcttttctcataaatttgtgccctctatttttactcccaatctgtgtaagatgaacaaatcttataagaccacatagctgactgtgatttcaggtggactccaggaaggagaaccaaagaaaactttcagtctttttaagatgaacaaatcttgtaagtccccacatagctgactgtgatttcaggtggactccaggaaggagaaccaaagaaaagttcaagtccaagcagaaaccgtgattccttccggatgatggctcatgagtgccatttaattggggtgccacctgctgtcctcagcaaatcccagctatatgtatatgttcgcattacaggctcattaacctaggctgacctctgcaaggatctcagaatattttctacagagaacatacatgataatatctgattttaggacaaaaaagtaattctcaatagcaagggaatggagtagggtagacagctagtaattaaactcacttgtgtgttaaaaataaattaaggaaaaaagaaaatgagagaacatattactaaataaagaaagcatacattaaatatttactatagttttacactaagagaataaaggaaatgcaataaagtggcctgaaaggtaaaggatgagatgtgtaaaagaggcggggaaagatgtgtcattttttttttactatgagcagcaatctgagaagataaaggaatcgagttatgggcagacatgatgtttgatcagtgttatttgttttcaaggcctgcctacttttttttcaaatattacaaacttttgaagtcacattctttttgttttttgctgtctgttactagatcgcacattctgtaaaggcagggaccatggtatgttgtttatctttggattctcagtgattgtcatatttatgtttgttgaatgaatcttaatccaagacttgggctccaggtatctttccattctggttccaaggagggaccttcctcacagcaggcgtgctgtgtggtctcacatctcactcctatatctttccctgtctgttactgccctcagtggagcccacagtgaccatctccccatccaggacagaggccctcaaccaccacaacctgctggtctgctcagtgacagatttctatccagcccagatcaaagtccggtggtttcggaatgaccaggaggagacaaccggagttgtgtccaccccccttattaggaacggtgactggaccttccagatcctggtgatgctggaaatgactccccagcatggagacgtctacacctgccacgtggagcaccccagcctccagaaccccatcaccgtggagtggcgtaaggggatattgagtttctgttactatgggccccacaagacaaagggcagagctcattctgacccatcccttcccatctcttatccctgatgtcactactgagctgggaatcacaggagactagagcacttcttcctccatggcaagtgcatcagaagaatcctgatctcatcacctttccagatgctagggaaattactctacatactgtttctctggatcccagtcctgatagctcggagggactgatcattagggctggtgactgggatcttagggtttaaggttatggatgagctcctgaggagtggagatctgcttctccactctctcacctactcactgtacccaaggacctattggctggcctttcccctcccttaggggtggtctgaatggaggactagtttcctttgacgccttcacctcctgaatctcagactggacttcagctcctcagcagggatgctatggggtgtggggacaaacactgacactcaggctctgcttcttaggggctcagtctgaatctgcccagagcaagatgctgagtggcattggaggcttcgtgctggggctcatcttcctcgggctgggccttattatccatcacaggagtcagaaaggtgaggaaccccaagggaaaaggggaagatgggctgtgacccagaccctctgttcagagaggtcctgtctctagatgtggctctttcctcctgaccccgagaggaagaaagctgagctggaggtgggaggagacaggacaagattggaggaggcattggaatctgattttactagttgaaaggtagccctgtcacacagctcactgatagagcttattgaaggacatccttacctttcatcattgtctcattggctcctttccaaaagcttcctccattaagagggtcagagccttggcctccttgccttctagtgacaatttcctttgttttaggggattttaaattagggtgcttaaggccttgaaggacatgggtgggaagagaatataactctaattaagtcacatgtgtcattttccattggggtgagagagtggctgtttgtgtaatgagacctttctctgcataacttccttttgtaagacctcaagggcctccaccagcaggtaatatttcagccgtgatccagtgtggggagggcacagatgtaagagggaagagcatgagctgagtgtacctgaccacagtggtctccgttcatggtatatttgctgctatgaggatcaagacttaggggcgaagtttgccagtttctaggaatctccagaggctgttccccagaaccaagccttaactttggtggcatcttcctgtgaaatgtgaagccagaaccacagcttaaatgttagacactaggatgatgcccactttgtgccacatgttggtggctactgcctgtaggcattttccagtgactgaaagaggctgctagtggtagggatgaggtatcatccaatttcctaaaaagaccgaacccttcatattccccagaagagtaacagctgttcccccacctccctcacatctgcatcaagctgaagttctgtgtcttcatgagctgatttcacctttgcacagatcttgggggaggtgatgacaatacactctggacctcagctttgtctgtctgaagctgcagggggcccctgaggggtgggggagatggcaggcccaccagcgtaccctgtgctgatcatccctcttctctcttctccagggctcctgcactgactcctgag

SEQ ID NO: 26 - DNA, 227 bp, synthetic construct
  misc_feature 1..227: note = Description of sequence: Gene: HLA-DQB1
  source 1..227: mol_type = other DNA
tgtaccagtttaaggccatgtgctacttcaccaacgggacggagcgcgtgcgttatgtgaccagatacatctataaccgagaggagtacgcacgcttcgacagcgacgtgggggtgtatcgggcggtgacgccgctggggccgcctgacgccgagtactggaacagccagaaggaagtcctggagaggacccgggcggagttggacacggtgtgcagacacaactac

SEQ ID NO: 27 - DNA, 5711 bp, synthetic construct
  misc_feature 1..5711: note = Description of sequence: Gene: HLA-DRA
  source 1..5711: mol_type = other DNA
tcactaatgtgcttcaggtatatccctgtctagaagtcagattggggttaaagagtctgtccgtgattgactaacagtcttaaatacttgatttgttgttgttgttgtcctgtttgtttaagaactttacttctttatccaatgaacggagtatcttgtgtcctggaccctttgcaagaacccttcccctagcaacagatgcgtcatctcaaaatatttttctgattggccaaagagtaattgatttgcattttaatggtcagactctattacaccccacattctcttttcttttattcttgtctgttctgcctcactcccgagctctactgactcccaaaagagcgcccaagaagaaaatggccataagtggagtccctgtgctaggatttttcatcatagctgtgctgatgagcgctcaggaatcatgggctatcaaaggtaggtgctgagggaatgaaatctgggacgatagactacgaagcattggagaaaagacctatggacatttggaagataatgtgtggagtgaaagaatagtgtgacaggtattatgtggtctcgacagaaagtataacaaattgtggtttggtggagttcttccctcaccacaaactgaagtaagtcaaatttggtttagagggtcaaaactgagttgtgtattgatgaatagcacggtcctgctacaagccaaactgggggtgggggtgggggtgggggaggaagaatattttctggcaagcattaacaagttatatttctgggctttaattattctttctggaaaattagtaaaattaaaaactaaaaaccacacatagttttgctagaattaaatgaaaaaaaaagttattagccctgttcttatctgaatacatgatacagtagttattttttggagtgtaaatcctgtcggtatatattgagcacatatattgtgttgaagattactagaaggaaaagtcatcaaaaagcaacaatttaccccaggaaaaggggagggaaggcatgctgatatgagttgcctcatgggacagtgatagccattccctgccttcccatctccatggtacagcagatcttatatcatgttaacttagtaatatttccaagagagtagaaaaataagtaaggaaatggggaatctgatattattgtctctcatctccagagcaacattggtgctgttgtaaagatgtactgtagaaaagtattcttcacccagcgtgacccccacagaaggtgtcaggtagacttgaaataagcaaagtaataacccagctcccatacccatagtggcaattgtagatttctattgccccaaaagagccatacatagggatacttacctagaaagacagaggatcttcccttggtttgtgaagaggcagctagtatatttgtgtgtgtttgcatagatgcaaacggtaaataaattcctaggtttatcaatacacagtcaaacattaaaatctctcatcttggctgggcacggtggctcacgcctgtaatcccagcactttgggaggccgaggcgggcggatcacgaggtcaagagatcgagaccgtcctgggcaacatggtgaaaccccgtctctactaaaaatacaaaaaattagctgggtatggtggcacacgcctgtagtcccagctactcgggaggctgaggcaggaggattgcttgagcccgggaggcggaggttgcagtgagctgagatggtgccactgcactccagcctggcgatagagcaagactccgtctcaaacaaccaaaccaaaacaaaacaaaatatctcaccttatctttgaagactaaggaaaaaaaaaatctcccactcatcgatacactccacagaggcagcatactctcccagtgtagctttctcttttcatgttcattattcccttggtgttggttattctcaatgtcaatcgtaacagaacatcttccataataacagtcccaatttaaggagcattaagataaaaggtggaattgccaaggtcaatccagacgagaaccttctcatagaggtaaccaccgtgtgggtttggatgctgggaagcagggggactatgacgctacaaggtctcagtcttaatttttggagtacttcagtccccaggtatattttccatagatttggcccttaaataaaaagaagcttctgactctaaaatgtaaacagtgcttgttacagtcttgttgatatattaagaaattactcaccttatctcatttaatcttaaaaacaaacccctgacaggatcaaaaccacagcaggactacataataggaaaactatacataaataggtagaataatctgctcaggatcactaggtaagttgctgaataagaattcaagatgtttttgatcccagagtttaaaacccaacctttcaaacagtgtttccttcttcttagagtacaatgttctgagaaagagatcctctggaattctggcctaagtgtatttaatgcccgggtaaagaaagtgagagaacatttctctttaggggctgctgctggatttctaaaaagaaaataatttctcagctagtaacatggagccaaacaacagcttcacaagactctgggttctttagccctcatctccttcaatccaccctctttataaccagtccttcttgtttttcccctcccagctttgttcagcagcatgcccttcacccagaccttgtcttgtcactcatccctactcgccatcattctttcattcctcttggcccaatctctctccaccacttcctgcctacatgtatgtaggttattcatttccctctcttgattccccccacccaactctctttctccatttcttgcctttcagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatggtacctccctctctgctgcactcctggacatgggaatccatagtttgaaagtagttgcttcagctctttgtgttagattattgtaactgattttccctccaagggcctaaccttgccattaacaagccccaaattctcatgccagaggtctgagaactttatgggtttgatcctatcttgttgtgctcaagtcttgtctctgtcatccatggtctcctacgaagtcattgccctaagttcatgctaggggagccagaagggaagtccttggatatcttatacctcaatattggctcaatttcttggggagggggtgctgtcagagattgttatctgaggatgtgacatagatttctcagggcacaatttcaactactttttcagctttagggtttttagatacgtttgtaccacaattgagcatgggagggagaggggtgagcctaagcagtgatggctgatttctgtcacgtctgtcatgtgtcccccagtacctccagaggtaactgtgctcacgaacagccctgtggaactgagagagcccaacgtcctcatctgtttcatcgacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactggggtatggaccaacactcaatctcctttatttcaaggtttcctcctatgatgcttgtgtgaaactcggtgttctaactgtttcataatatctgctacaattaatataactgtcttctcctactatccagcttcctcctttttttaatctgtaattctctcaatacatcattctgtcttcctcttctttaatctatgaataacttttctctttattaagaaccctacatttgattctgagtgttacttcttcccacactcattaccatgtactctgccttatctccccccagagtttgatgctccaagccctctcccagagactacagagaacgtggtgtgtgccctgggcctgactgtgggtctggtgggcatcattattgggaccatcttcatcatcaagggagtgcgcaaaagcaatgcagcagaacgcagggggcctctgtaaggcacatggaggtgagttaggtgtggtcagaggaagacatatatggagatatctgagggaggaaaacagggtggggaaaggaaatgtaatgcatttaagagacaaggtaggaacagatgtggctcttgatttctctttgctagaatgaatcagacattggtatcatctggtatcccaaagcttcagggtctgtcatccctttctatagacgggcaccttgatcacggctccagtcttagaaatcatctccagtacctaaaaccattgtttcacattagaatactgagtctagggatctagaaaatacattagaatatggagtctagggatctagaaaatactgagtctagggatctagaaaaataagcctcaagatttgggcacatcctagcttgtatttcctggggcaggtcatcagttcagaagcatttccagatcctggctcctttcaggttagggtcaattcattgcatgaaatgggaatctcttagaggccaatgcctgcttttgcttctttagtctcaaatgtagtatgagaaactctaaaaaaaggtaaagcatggttgcttattatgttcagttggagagtaggaactaactgtatacagttagttcatgttggaaaggttagatgaacattgaaagaattttgcaaagtcaaaggattaagagagaagaggaaggaatctgaagcaaggagctcaaaacggatcttaaattccttggtaactatgtgtgtcttgctataggtgatggtgtttcttagagagaagatcactgaagaaacttctgctttaatgactttacaaagctggcaatattacaatccttgacctcagtgaaagcagtcatcttcagcgttttccagccctatagccaccccaagtgtggttatgcctcctcgattgctccgtactctaacatctagctggcttccctgtctattgccttttcctgtatctattttcctctatttcctatcattttattatcaccatgcaatgcctctggaataaaacatacaggagtctgtctctgctatggaatgccccatggggcatctcttgtgtacttattgtttaaggtttcctcaaactgtgatttttctgaacacaataaactattttgatgatcttgggtggaatttttggtgtttaagccagttctttgggtggcggtggggggtggggagtcggtcctggggaatatatgtgatcctttcccggtaaaatatctgaatgttgaatttatcttataaattctagaattcatcagacatatcccggttcatttgggcttggtctcattttgtgcatctgcaggcaaccctcttgttgtggtctagtcctcatcaggaaaacctaaagtggggttggtttgttgggagatctcta

SEQ ID NO: 28 - DNA, 5141 bp, synthetic construct
  misc_feature 1..5141: note = Description of sequence: Gene: HLA-DRB1
  source 1..5141: mol_type = other DNA
tacgtctgagtgtcatttcttcaatgggacggagcgggtgcggttcctggacagatacttccataaccaggaggagttcgtgcgcttcgacagcgacgtgggggagtaccgggcggtgacggagctggggcggcctgctgcggagcactggaacagccagaaggacctcctggagcggaggcgggccgaggtggacacctattgcagacacaactacggggttgtggagagcttcacagtgcagcggcgaggtgagcgcggcgcggggcggggcctgagtccctgtgagctgggaatctgagtgtgtgtgtgtgtgtgtgtgtgtgtgtgtgtgtgtgtgtgtgtgtgtgtgtgtgtgagagagagagagagagagagagacagagagagagagagcgccatctgtgagcatttagaatcctctctatcctgagcaaggagttctgagggcacaggtgtgtgtgtagagtgtggatttgtctgtgtctgtgaggctgttgtgggaggggaggcaggagggggctgcttcttattcttggaggactctgtggggaggtgacaagggaggtgggtgcgggcggctggagagagaggtgaccttgattgtcctgggtccttagagatgcagggaagggaaatgtaaggggtgtgtggttggggtgaaggtttaggggaggagagctgaggggtaaggaaggtttgggataatgtgaggaggccggttccagactgtccctggcacacacccttcatgtaatctctgaaataaaagtgtgtgctgtttgtttgtaaaagcattagattaatttctaggggaattgaggagacctctgaggcatctctgaagcttctttaggtctaaatttcttgctagttttttgttttttattgtgtatatttttacatagtagaaatgactgtgaaactaactttttgaattaaagttttaacacagttactattttattataatgctaatagttttctagtagttacatattattcttttatatataatagttgtgacacaacttacctcactttcccctttgttgacctttattatgacattcaccaaaagttgaaaatgtatgtttctggttaatttttaatttatatttttttcatttataattcttttgaattattttgacctatttattggccagttttaataactgctgtaagaattccctattgtatttggtagggaatggacaatgatctactgcctaatatctcgagggcttagtatttttctcagtgactttgtgggttctttgtactgtgagattattaacactttattgatatttgattcagcatttgctccagtttgtggtttgtatgttgattttgaaaattcttttccatgttaagaatttgaacatttttatataataaaatatgttgcaaaatttttattaatgatttacaatccatcttaaatctgccattttgtggtattgttgtctccaggtttctccttacttctaaaaaaaattgcatttattgagagtctgctagtgttagggattttcctgggcataagcaccccaagtgacgagtcccagacactgccttaatccaaatgtgattctggaaagaaaaatcattttacaatgataggcctaataataattaagcttgtgttgcatgggagatgcattgatcagctaaatgtaaatataagaactttcaaaactaaaatgacgttccttaatccttctctctgctttatgaatcatgcttttctgggaaagtaaaaatttggagaatcatttctgtctgtcccaccttcccaggggcagaaccatttctgtagtgttctaaggtgtgagtgcatggcagtagtattcctaaaaattcatattcggtttcgtcatgtacccaactctgtcccgttatctatccacattgttttaaatcatatatttctgtcaaggtgtacaaggatgataaataggtgccaagtggagcacccaagtgtgatgagccccctcacagtggaatggagtgtgaagctttatgacctcataaattgaaggttatcttcagtcattgttttatatattttacatgcattaatcctcatataatcccaagaggtaaattagtataattatccttcattataggtgacaaagttgagacacagaagaatcaaactcttaaggcagaccttggatttgaaccaggcaacctggctcagatatccgttttaattactacactctgtactttcaaagatttgtaaacactttgacaatgcatgacaatttcaagctatgaagaaacaaacacaatttttcacaatatctctcaaatctaatgggtcctcactatcaagattaagttccaggctgatgacactgtaaggccacatggccagctgtgctggaggcctggtcaaggtcagagcctgggtttgcagagaagcagacaaacagccaaacaaggagacttactctgtcttcatgactcattccctctaccttttttctcctagtccatcctaaggtgactgtgtatccttcaaagacccagcccctgcagcaccacaacctgctggtctgttctgtgagtggtttctatccaggcagcattgaagtcaggtggttccggaatggccaggaagagaagactggggtggtgtccacaggcctgatccacaatggagactggaccttccagaccctggtgatgctggaaacagttcctcggagtggagaggtttacacctgccaagtggagcacccaagcgtgacaagccctctcacagtggaatggagtgagcagctttctgacttcataaatttctcacccaccaagaaggggactgtgctcatccctgagtgtcaggtttctcctctccgacatcctattttcatttgctccatgttctcatctccatcagcacaggtcactgggggtagccctgtaggtgtttctagaaacacctgtacctcctggagaagcagtctcgcctgccaggcaggagaggctgtccctcttttgaacctccccatgatgtcacaggtcagggtcacccaccctccccgggctccaggcactgcctctgggtctgagactgagtttctggtgctgttgatctgagttatttgttgtgatctgggaagaggagaagtgtaggggccttcctgacatgaggggagtccaatctcagctctgccttttattagctctgtcactctagacaaactacttagcctcattgagtctcaggctttctgtggatcagatgttgaactcttgccttacatcaaggctgtaatatttgaatgagtttgatgtctgaaccttgtaactgttcagtgtgatttgaaatcctttttttctccagaaatggctagttattttagttcttgtggggcagacttcttccccattttcaaagctctgaatcttagagtctcaattaaagaggttcaatttggaataagcatcactaaacctggcttcctctctcaggagcacggtctgaatctgcacagagcaagatgctgagtggagtcgggggctttgtgctgggcctgctcttccttggggccgggctgttcatctacttcaggaatcagaaaggtgaggagcctttggtagctggctctctccataggcttttctggaggaggaactatggctttgctgaggttagttctcagtatatgagtggccctgaataaagcctttctttccccaaacggctctaatgtcctgctaatccagaaatcatcagtgcatggttactatgtgaaagcataatagcttgtggcctgcagagacaagaggaaggttaacaagtaggggtcctttggtttgagatcttggagcagattaaggaagagccactaagactaatggaattacactggatcctgtgacagacacttcacccttcatgggtcacatggtctgtttctgctcctctctgccctggctggtgtgggttgtagtgacagagaactctccggtgggagatctggggctgggacattgtgttggaagacagatttgcttccataaattttaagtgtatatattttcctctttttcccaggacactctggacttcagccaagaggtaataccttttaatcctcttttagaaacagatacggtttccctagtgagaggtgaagccagctggacttctgggtcgggtagggacttgcagaacttttctgtcttaggagaggtttctaaatgcaccaatcagtgctctgtaaaaacacacccattggcactctgtggctagatagatgtttgtaaaatggactaatcagcactctgtaaaatggagcaatccacactctgtaaaatgggccaatcaatgctctttaaaatggaccaatcagcaggacatgggcggggacaaataagggaatacaagctggccaccccagccagcagcagcaacccgctcaggtcgccttccatgctgtggaagctttgttcttttgctcttcacaataaatcttgctgttgctcactcttcgggtctgtgccacctttaagagctgtaacactcactgtgaagattcgcggcttcattcttgaagtcagcaaaaccacgaacccaccggaaggaacaaactctggacacactagaattgatggtagaggtgataaggcatgagacagaaataataggaaagactttggatccaaatttctgatcaggcaatttacaccaaaactcctcctctccacttagaaaaggcctgtgctctgtgggactattggctctgggagactcaggaacttgtttttcttcttcctgcagtgctctcatctgagtccctgaaagagaggaaaagaaactgttagtagagtcaggttgaaaacaacactctcctctgtcttttgcaggattcctgagctgaagtgcagatgacacattcaaagaagaactttctgccccagctttgcaggatgaaaagcttttcctcctggctgttattcttccacaagagagggctttctcaggacctggttgctactggttcagcaactgcagaaaatgtcctcccttgtggcttcctcagctcctgcccttggcctgaagtcccagcattggtggcagcgcctcatcttcaacttttgtgctcccctttgcctaa

SEQ ID NO: 29 - DNA, 5474 bp, synthetic construct
  misc_feature 1..5474: note = Description of sequence: Gene: HLA-DRB1
  source 1..5474: mol_type = other DNA
gagtgtcatttcttcaatgggacggagcgggtgcggttcctggacagatacttctataaccaggaggagtccgtgcgcttcgacagcgacgtgggggagtaccgggcggtgacggagctggggcggcctgacgctgagtactggaacagccagaaggacctcctggaagacaggcgcgccgcggtggacacctactgcagacacaactacggggttggtgagagcttcacagtgcagcggcgaggtgagcatggtggggggcggggcctgggtccttgtgagctgggaatctgagtgtgtgtgtgtgtgtgtgtgtgtgtgtgtgtgtgtgagagagagagagagaggaagagagagagagagcgccatctgtgagcatttagaatcctctcagtccggagcaagcagttctgagagcacaggtgtgtgtgtaaagtgtggatttgtctgtgtctgtgtgtctgttgtgggaggggaggcaggagggggctgtttcttatccttggagacctctgtggggaggtgacaagggaggtgggtgctgggggctggagagagaggagaccttgattatctcggtccttagagatgcagggaagggaaatgtaaggggtgtgtggttggggtgaaggtttaggggaggactgctgaggggtaaggcaggtttgggataatgtgaggaggccagttccagactctccctggcatacacccttcgtgtaatctctgaattaaaagtgtgtgctgtttgtttgtaaaagcaatagattaatttctagaggaactgagtagacctctgaggcacctctgaagcttcattagatctaaatttcttcctagttttttgtttttttcagtgtgtatatttttacatagtagaaatgactgtgaaactaactttttgaattaaagttttgacacggttactattttattataatgctggtagttttctagtagttacatattattcttttatatataatatttgtgacacaactcacctcactttcccgtttgttgacctttattatgacattcaccagaagttgaaattgtgtgtttctggttaatttttaatttatattttttatttgtaattcctttgaattattttgccctatttattggccgattataattattgctctaagaattccctattgtatttggtaggtaatggacaatgatctacggtctaatatcttgagggcttagtatttttctcagtgactttctgcgttctttgtactataagattattaacactttattgatatttgatccagcatttgctccagtttgtggtttgtatgtggattttgaaaattcttttccatgttaagaatttgaacatttttatttaataaaatatattgcaacatttttattaatgatctacaatccatctgaaatctgccattttgtggcattgttgtctccaggtttctccttacttctaaaaaaatagttgtatttattgagagtatgctagtgtcggggattttcctgggcataagcaccccaagtaacgagtcccagacactgccttaatccaaatgtgattctggaaagaaaaatcattttacaatgataggcctaataataattatgcttgtgttgcacgggagatgcattgatcagctaaatgtaaatataagaactttcaaaacttaaatgacgttccctaatccttctctctgcttcaggactcatgcttttctaggaaagtaaaaatttggagaatcatttctgtctgtcccaccttcccaggggcagaacaatttctgttgtgttctaaggtgtgagtgcatgggagtagtattcctaaaattcatacttggtttcctcatgtacccaactctgtccctttatctatgcatattgctttaaatcatatttttctgtcaaggtgtacaaggatgataaataggtgccaagtggagcactcaagtgtgatgagtgccctcacagtggaatggagtgagaagctttctgacctcataaactgaaggctatcttcagtcattgttttatatattttacgtgcattaatcctcatataaccccaagaggtaaattagtataattatccttcattgtaggtgacaaagttgagacacagaagaatcaaaaaactcttccaggatcaaccagtaaaaggcagaccttggattcgaaccaggcaacctggctcaaatatcagttttaattactacactctgtactttccaagatttgtaaacagtttgacaatgcatgccgatttaaaactatgaagaaacaaacacaatttttcacaacacctctcaaatctaatgggtcctcactgtcaagattaaattccaggctgatgacactgtaaggtcacatggccagctgtgctataggcctggtcaaggccagagcctgggtttgcagagaagcagacacacagccaaaccaggagacttactctgtcttcctgactcattccctctacgttgtttttctcctagtccaacctaaggtgactgtatatccttcaaagacccagcccctgcagcaccacaacctcctggtctgctctgtgagtggtttctatccaggcagcattgaagtcaggtggttcctgaacggccaggaagagaaggctgggatggtgtccacaggcctgatccagaatggagactggaccttccagaccctggtgatgctggaaacagttcctcgaagtggagaggtttacacctgccaagtggagcacccaagcgtgacaagccctctcacagtggaatggagtgagcagctttctgacttcctaaatttctcacccactaagaaggggaccgtgctaatccctgagtgtcaggttcctcctctcccacatcctattttaatttgctccatgttctcatctccatcagcacaggtcactgggggtagccctgtaggtgtttctagaaacacctgtacctcctggagaagcagtctcgcctgccaggcgggagagactgtccctcttttgaacctccccatgattttgcaggtcagggtcacccactctcccaggctccaggccctgcctctgggtctgagactgagtttctggtgctgttgctctgagttattttttgtgatctgggaagaggagaagtgtaggggcctacctgacatgaggggagtccaatctcagccctgctttttattagctttgtcactctagacaaactacttatcctcattgagtctcaggctttctgtggatcagatgttgaacttgtgccttacatcaaggctgtaatatttgaatgagtttgatgtctgaacatcgtaactgttcagtgtaatttgaaatcctttttttctcctgaaatggctagttattttagttcttgtgaggcagctttctgccccattttcaaagctctgaatcttagagtctcaagtaaaaaggttcaatttggaataaacatcactaaacctggcttcctctctcaggagcacggtctgaatctgcacagagcaagatgctgagtggagtcgggggctttgtgctgggcctgctcttccttggggccgggctgttcatctacttcaggaatcagaaaggtgaggagcctttgggagctggctctctccataagcttttctggaggaggaactatggctttgctgaggttagttctcagtatatgagtggccctgaataaagcctttctttccccaaacggctctaatgtcctgctaatccagaaatcatcagtgcatggttactatgtgaaagcataatagcttgtggcctgcagagacgagaggaaggttaacaagtaggggtcctctggtttgagatcctggagcaaattaaggaagagccactaaggctaatggaattacactggatcctgtgacagacacttcaggcttcatgggtcacatggtctgtttctgctcctttctgccctggttggtgtgggttgtggtgttagagaaatctcaggtgggagatctggggctgggacattgtgttggaggacagatttgcttcaataacttttaagtgtatatcttttcctctttttcccaggacactctggacttcagccaacaggtaataccttttaatcctcttttagaaacagacacagtttccctagtgagaggtgaagccagctggacttctgggtggggtggggacttggagaacttttcttacaagaggtttctttttttttttttttttgagacggagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaattttttgtatttttagtagagacggggtttcaccgttttagccaggatggtctcgatctcctgacctcgtgatccgcccgcctcggcctcccaaagtgctgggattacaggtgtgagccaccgcgcccggcctacaagaggtttctaaatgcaccaatcagtgctttgtaaaaacacaccaataggttctctgtggctagctggatgtttgtaaaatggaccaattcgcactctgtaaaatagaccaatcagcactctttaaaatggaccaatcagcacttttaaaatgggccaatcaccactctttaaaatggaccaatcagcactctttaaaatggaccaatctgcaggacatgggcagggacaaatacaggaataaaagctggccaccccaaccagcagtggcaacccactcaggtcctcttccctgctgtggaagttttgttcttttgatcttcacaataaatcttgctgctgcccactctttgggtccctgccgcctttaagagctgtaacactcactgtgaaggtctgcaggaagtcagcgagaccacaaacccaccggaaggaacaaactcaggacacaccagaatgatggtagaggtgataaggcatgagacagaaataataggaaagactttggatccaaatttctgatcaggcaatttacaccaaaattcctcctctccacttagaaaaggcctgtgctctgcgggactattggctcaggggagactcaggaacttctttttcttcttcctgcagtgctctcatctgagcccttgaaagaggggaaaagaaactgttagtagagccaggttgaaaacaacactctcctctgtcttttgcaggattcctgagctgaaatgcagatgaccacattcaaggaagaactttctgccccggctttgcaggatgaaaagctttcctgcttggcagttattcttccacaagagagggctttctcaggacctggttgctactggttcggcaactgcagaaaatgtcctcccttgtggcttcctcagctcctgcccttggcctgaagtcccagcattgatggcagcgcctcatcttcaacttttgtgctcccctttgcctaa

SEQ ID NO: 30 - DNA, 5153 bp, synthetic construct
  misc_feature 1..5153: note = Description of sequence: Gene: HLA- DRB3
  source 1..5153: mol_type = other DNA
taagtctgagtgtcatttcttcaatgggacggagcgggtgcggttcctggagagatacttccataaccaggaggagttcgtgcgcttcgacagcgacgtgggggagtaccgggcggtgacggagctggggcggcctgtcgccgagtcctggaacagccagaaggacctcctggagcagaagcggggccaggtggacaattactgcagacacaactacggggttgtggagagcttcacagtgcagcggcgaggtgagcatgtcaggggcggggcctgggtccctgtgagctggaaatctgagagtgtgtgtgtgtgtatgtgtgtatgtgtgtgtgtgagagagagagacagagaggaagagagagacagagagagggagcgcgccatctgtgagcatttagaatcctctcaatcccgagcaagcagttctgaaggcacaggtgtgtgtgtagagtgtggatttgtctgtgtctgtgtgcctgttgtgggaggggaagcagaagggggctgcttcttatccttggagacttctgtggagaggtgaaaagggaggtgggtgcgggcggctggagagagaggagaccttgattgtcctgggtccttagagatgcagggaaggaaagtgtaaggtgtgtgtgggtggggtgaaggtttaggggaggagagctgaggggtaaggaaggtttgggataatgtgaggaggccggttccagactgtccctggcacacacccttcatgtaatctctgaaataaaagtgcgtgctgtttgtaaaagcattagattaagttctaggggaattgagtagacctctaaggcacctctgaaacttctttaggtataaatttcttgctagttttttgttttttagtgtgtatatttttacatagtagaaatgacagtgaaactaacttttgaattaaagttttaacacagttactgtattattataatgctaatagttttctagtagttacatattattcttttatatataatagttgtgacacaacttagggatcactttcccctttgttgacctttattatgacattcaccaaaagttgaaaatgtatgtttctggttaatttttaatttatattttttcatttgaaaaaatgtggagaggtctgtttttcattcaaaagagttcttttgaattattttgaccgatttattggtcaattttaataactgctctaagaattccctattttatttggtaggtaatggacaatgatctactgcctaatatctctagggcttagtatttttctcagtgactttgtgggttctttgtactgtaagattattaacactttattgatatttgattcagcatttgctccagtttgtggtttttatgttgattttgaaaattcttttccatgttaagaatttgaacatttttatttaataaaatgtattgcaaaaattttattaatgatttacaatccatcttaaatctgccattttgtggtatttttgtctctaggtttctccttacttctaaaaacattgtatttattgagagtatgctaatgtcggggattttcctgggcataagcatcacaagtaatgagttccagaccctgccttaatccaaatgtgattctggaaagaaaaatcattttacaattggcctgataataattctgcttgtgttgcacgggggatacattgagcagctgaatgaaaatataagaactttcaaaactaaaatgacgctcctaaatccttctgtctgctttaggactcatgcttttctaggaacgtaaaaatttggagaatcatttctgtctgtcccaccttcccaggggcagaaccatttctgtggtgttctaaggtgtgagtgcatggcagcagtattcctaaaaattcatactctgttttctcatgtacccaactctgtccctttgtctatgtacattgctttaaatcatatttttctgtcacggtgtacaaggatgataaataggtgccaagtggagcacccaagtgtgatgagcccccttatggtggaatggagcgagaagctttctgacctcataaattgaaggctatcttcactcattatgtattttacgtaaattaatcctcatataaccccaagaggtaaattagtataattatccttcattgggggtgacaaagttgagacacagaagaatcaactcttccaggatcaaccagtaaaaggcagaccttggatttgaaccatgcaacctggctcagatatcagttttaattactacactctgtactttcaaaaatttgtaaacactttgacaatgcatgccaatttcaagctatgaagaagcaaacgcaatttttcacatctctcaaatctaatgggtcctcactatcaagattaaattccaggctgatgacactgtaaggccacatggccagctgtgctggaggcctgttcaaggtcagagcctgggtttgcagagaagcagacaaacaccaaaaccaggagaattactctgtcttcctgactcattccctctacctttttttctactagtccatcctcaggtgactgtgtatcctgcaaagacccagcccctgcagcaccacaacctcctggtctgttctgtgagtggtttctatccaggcagcattgaagtcaggtggttccggaatggccaggaagagaagactggggtggtgtccacaggcctgatccataatggagactggaccttccagaccctggtgatgctagaaacagttcctcggagtggagaggtttacacttgccaagtggagcacccaagcgtaacaagccctctcacagtggaatggagtgagcagctttctgatttcatacatttctcacccaccaagaaggggactgtgctaatccctgagtgtcaggtttctcctctcccacgtcctattttcatttgttccatgttctcatctccatcagcacaggtcactggggatagccctgtaacagtgtgtagaaacacctgtaccccctgtggaatcagtcatgcttttttttttttgaacctccccatgatgtcacaggtcagggtcacccgctctccccaagctccaggccctgcttttgggtctgagactgagtttctggtgctgctgctctgagttacttgttttgatccgggaagaggggaagcataggggcctacctgacatgaggggagtctaatctcagctctaccttttattagctctgtcactctagacaaactacttagcttcattgagtctcaggctttctgttgatcagatgttgaacgcttgccttacatcaatgctgtaatatttgaatgagtttgatgtcggaaccttgtaactgttcagtgtgatttgaaatcctttttttctcccgaaatggctagttattttagttcttgtggggcagccttcttccccattttcaaagctctgaatcttagagtctcaattaaagaggttcaatttggaataaacgtcactaaacctggcttcctctctcaggagcacggtctgaatctgcacagagcaagatgctgagtggagtcgggggctttgtgctgggcctgctcttccttggggccgggctgttcatctacttcaggaatcagaaaggtaaggagtctttggtagctggctctctccgtaggcttttctggaggaggaactatagctttgctgaggttagttctcagtatatgagtggccctgaataaagcctttctttccccaaacggctctaatgtcctgctaatccagaaatcatcagtgcatggttactatgtgaaagcataatagcttgtggcctgcagagacaagaggaaggttaacaagtaggggtcctttggtttgagatcttggagcaaattaaggaagagccactaaagttaatggaattacactggatcctgtgacagacacttcatgcttcatgggtcacatggtctgtttctgctcctctctgccctggttggtgtgggttttggtgttagagaactctccggtgggagatctgggactgggatattgtgttggaggacagatttgcttcaatatcttttaagtgtatatcttttcctctttttcccaggacactctggacttcagccaacaggtaataccttttaatcctcttttagacacagattcagtttctctagtgagaggtgaagccagctggacttctgggttgggtggggacttggagaacttttctgtcttacaagaggtttctaaatgcaccaatgagtgctctgtaaaaacacaccaatgagtgctctgtaaaaattgacactctgtggcttgctagatgtttgtaagatggaccaatcagcactctgtaaaatggaccaatccacactctgtaaaatggaccaatcagcactctttaaaatggaccaatcagcaggatacgggtggggacaaataagggaataaaagctggtcaccctagccagcaccggcaacctgcttaggtccttttctatgctgtggaaggtctgttctttcactcttcacaataaatcttgtgctcactctttgggtccgtgccacctttaagagctataacactcactgcaagggtctgtggcttcactcttgaagtcagccagaccgtgaacccaccggaaggaacaaactctggacacactagaatggtggtagaggtgataaggcatgagacagaaataataggaaagactttggatccaaatttctgatcaggcaatttacaccaaaactcctcctctccacttagaaaaggcctgtgctctgcaggactattggctctgggagactcaggaacttgtttttcttcttcctgcagtgttctcatctgagtccttcaaagagggggaaagaaacgttagtagagccaggttgaaaacaacactctcctctgtcttttgcaggattcctgagctgaagtgcagatgacaatttaaggaagaatcttctgccccagctttgcaggatgaaaagctttcccgcctggctgttattcttccacgagagagggctttctcaggacctggttgctactggttcagcaactgcagaaaatatcctcccttgtggcttcctcagctcctgcccttggcctgaagtcccagcattgatggcagcgcctcatcttcaacttttgtgctcccctttgcctaa

SEQ ID NO: 31 - DNA, 4930 bp, synthetic construct
  misc_feature 1..4930: note = Description of sequence: Gene: HLA- DRB3
  source 1..4930: mol_type = other DNA
taagtctgagtgtcatttcttcaatgggacggagcgggtgcggtacctggacagatacttccataaccaggaggagttcctgcgcttcgacagcgacgtgggggagtaccgggcggtgacggagctggggcggcctgtcgccgagtcctggaacagccagaaggacctcctggagcagaagcggggccgggtggacaattactgcagacacaactacggggttggtgagagcttcacagtgcagcggcgaggtgagcatgtcggggggcggggcctgggtccctgtgagctggaaatctgagagagagtgtgtgtgtgtgtgtgtgtgtgtgtgtgagagagagagagagagaggaagagacagagagagggagcgcgccatctgtgagcatttagaatcctctcaatcccgagcaagcagttatgaaggcacaggtgtgtgtgtagagtgtggattggtctgtgtctgtgtgcctgttgtgggaggggaagcaggagggggctgcttcttatccttggagacttctgtggagaggtgacaagggaggtgggtgcgggcggctggagagagaggagaccttgattgtcctgggtccttagagatgcagggaaggaaagtgtaaggtgtgtgtgggtggggtgaaggtttaggggaggagagctgaggggtaaggaaggtttgggataatgtgaggaggccggttccagactgtccctggcacacacccttcatgtaatctctgaaataaaagtgcgtgctgtttgtaaaagcattagattaagttctaggggaattgagtagacctctaaggcacctctgaaacttctttaggtataaatttcttgctagttttttgtttttctagtgtgtatatttttacatagtagaaatgacagtgaaactaacttttgaattaaagttttaacacagttactgtattattataatgctaatagttttctagtagttacatattattcttttatatataatagttgtgacacaacttagggatcactttcccctttgttgacctttattatgacattcaccaaaagttgaaaatgtatgtttctggttaatttttaatttatattttttcatttgaaaaaatgtggagaggtctgtttttcattcaaaagagttcttttgaattattttgaccgatttattggtcaattttaataactgctctaagaattccctattttatttggtaggtaatggacaatgatctactgcctaatatctctagggcttagtatttttctcagtgactttgtgggttctttgtactgtaagattattaacactttattgatatttgattcagcatttgctccagtttgtggtttgtatgttgattttgaaaattcttttccatgttaagaatttgaacatttttatttaataaaatgtattgcaaaaattttattaatgatttacaatccatcttaaatctgccattttgtggtatttttgtctctaggtttctccttacttctaaaaacattgtatttattgagagtatgctaatgtcggggattttcctgggcataagcaccacaagtaatgagttccagaccctgccttaatccaaatgtgattctggaaagaaaaatcattttacaattggcctgataataattctgcttgtgttgcacgggggatacattgagcagctgaatgaaaatataagaactttcaaaactaaaatgacgctcctaaatccttctgtctgctttaggactcatgcttttctaggaacgtaaaaatttggagaatcatttctgtctgtcccaccttcccaggggcagaaccatttctgtggtgttctaaggtgtgagtgcatggcagtagtattcctaaaaattcatactcggttttctcatgtacccaactctgtccctttgtctatgtacattgctttaaatcatatttttctgtcacggtgtacaaggatgataaataggtgccaagtggagcacccaagtgtgatgagcccccttacggtggaatggagtgagaagctttctgatctcataaattgaaggctatcttcagtcattatgtattttacataaattaatcctcatataaccccaagaggtaaattagtataattatccttcattgggggtgacaaagttgagacacagaagaatcaactcttccaggatcaaccagtaaaaggcagaccttggatttgaaccatgcaacctggctcaggtatcagttttaattactacactctgtactttcaaaaatttgtaaacactttgacaatgcatgccaatttcaagttatgaagaagcaaacgcaatttttcacatctctcaaatctaatgggtcctcactatcaagattaaattccaggctgatgacactgtaaggccacatggccagctgtgctggaggcctggtcaaggtcagagcctgggtttgcagagaagcagacaaacagccaaaccagcagaattactctgtcttcctgactcattccctctacctttttttctcctagtccatcctcaggtgactgtgtatcctgcaaagacccagcccctgcagcaccacaacctcctggtctgctctgtgagtggtttctatccaggcagcattgaagtcaggtggttccggaacggccaggaagagaaggctggggtggtgtccacgggcctgatccagaatggagactggaccttccagaccctggtgatgctagaaacagttcctcggagtggagaggtttacacttgccaagtggagcacccaagcgtaacgagcgctctcacagtggaatggagtgagcagctttctgatttcatacatttctcacccaccaagaaggggactgtgctaatccctgagtgtcaggtttctcctctcccacgtcctattttcatttgctccatgttctcatctccatcagcacaggtcactggggatagccctgtaacagtgtgtagaaacacctgtactccctgtggaatcagtcatgctttttttttttttgaacctccccatgatgtcacaggtcagggtcacccgatctccccaagctccaggccctgcttctgggtctgagactgagtttctggtgctgctgctctgagttacttgttttgatctgggaagaggggaagcataggggcctacctgacatgaggggagtccaatctcagctctgccttttattagctctgtcactctagacaaactacttagcttcactgagtctcaggctttctgtggatcagatgttgaacgcttgccttacatcaaggctgtaatatttgaatgagtttgatgtcggaaccttgtaactgttcagtgtgatttgaaatcctttttttctcccaaaatggctagttattttagttcttgtggggcagccttcttccccattttcaaagctctgaatcttagagtctcaattaaagaggttcaatttggaataagcatcactaaacctggcttcctctctcaggagcacggtctgaatctgcacagagcaagatgctgagtggagtcgggggctttgtgctgggcctgctcttccttggggccgggctgttcatctacttcaggaatcagaaaggtgaggagcctttggtagctgggcctttccataggcttttctgcaggaggaaatagagctttgctgaggttagttctcagtatatgagtggctctgaataaagcctttctttccccaaacggctctaatgtcctgctaatccagaaatcatcagtgcatggttactatgtgaaagcataatagcttgtggcctgcagagacaagaggaaggttaacaagtaggggtcctttggtttgagatcttggagcaaattaaggaagagacactaaagttaatggaattacactggatcctgtgacagacacttcatgcttcatgggtcacatggtctgtttctgctcctctctgccctggttggtgtgggttttggtgttagaactctccggtgggagatctgggactgtgatattgtgttggaggacagatttgcttcaatatcttttaagtgtatatcttttcctctttttcccaggacactctggacttcagccaacaggtaataccttttaatcctcttttagacacagattcagtttctccagtgagaggtgaagccagctggacttctgggttgggtggggacttggagaacttttctgtcttacaagaggtttctaaatgcaccaatgagtgctctgtaaaaacacaccaatgagtgctctgtaaaaattgacactctgtggctcgctagatgtttgtaagatggaccaatcagcactctgtaaaatggaccaatccacactctgtaaaatggaccaatcagcactctttaaaatggaccaatcagcaggatacgggcggggacaaataagggaataaaagctggtcaccctagccagcacctgcaacctgcttaggtccttttctatgctgtggaagttttgttcttttgatcttcacaataaatcttgtgctcactctttgggtccgtgccacctttaagagctataacactcactgcaagggtctgtggcttcactcttgaagtcagccagaccctgaacctaccggaaggaacaaactcaggacacactagaatgatggtagaggtgataaggcatgagacagaaataataggaaagactttggatccaaatttctgatcaggcaatttacaccaaaactcctcctctccacttagaaaaggcctgtgctctgcaggactattggctctgggagactcaggaacttgtttttcttcttcctgcagtgttctcatctgagtccttcaaagagggggaaagaaacgttagtagagccaggttgaaaacaacactctcctctgtcttttgcaggattcctgagctgaagtgcagatgacaattta

SEQ ID NO: 32 - DNA, 930 bp, synthetic construct
  misc_feature 1..930: note = Description of sequence: Gene: HLA-DRB4
  source 1..930: mol_type = other DNA
gttcgttgcaacaaattgatgagcaatgcttttttataatgccaactttgtacaaaaaagttggcatggtgtgtctgaagctccctggaggctcctgtatggcagcgctgacagtgacattgacggtgctgagctccccactggctttggctggggacacccaaccacgtttcttggagcaggctaagtgtgagtgtcatttcctcaatgggacggagcgagtgtggaacctgatcagatacatctataaccaagaggagtacgcgcgctacaacagtgacctgggggagtaccaggcggtgacggagctggggcggcctgacgctgagtactggaacagccagaaggacctcctggagcggaggcgggccgaggtggacacctactgcagatacaactacggggttgtggagagcttcacagtgcagcggcgagtccaacctaaggtgactgtgtatccttcaaagacccagcccctgcagcaccacaacctcctggtctgctctgtgaatggtttctatccaggcagcattgaagtcaggtggttccggaacggccaggaagagaaggctggggtggtgtccacaggcctgatccagaatggagactggaccttccagaccctggtgatgctggaaacagttcctcggagtggagaggtttacacctgccaagtggagcatccaagcatgatgagccctctcacggtgcaatggagtgcacggtctgaatctgcacagagcaagatgctgagtggagtcgggggctttgtgctgggcctgctcttccttgggacagggctgttcatctacttcaggaatcagaaaggacactctggacttcagccaacaggactcttgagctgcccaactttcttgtacaaagttggcattataagaaagcattgcttatcaatttgttgcaacgaac

SEQ ID NO: 33 - DNA, 5139 bp, synthetic construct
  misc_feature 1..5139: note = Description of sequence: Gene: HLA-DRB4
  source 1..5139: mol_type = other DNA
taagtgtgagtgtcatttcctcaatgggacggagcgagtgtggaacctgatcagatacatctataaccaagaggagtacgcgcgctacaacagtgacctgggggagtaccaggcggtgacggagctggggcggcctgacgctgagtactggaacagccagaaggacctcctggagcggaggcgggccgaggtggacacctactgcagatacaactacggggttgtggagagcttcacagtgcagcggcgaggtgagcatggtggagggcggggcctgggtccctgtgagcggggaatctgtgtatgtgtgtgtgtgtgtgtgtgtgtgagagagagagagagagagagagacagagaaagagagagagagcgtgcgccatctgtgagcatttagaatcctctctatcctttcttatggctgcgcagtattccctggtgtataggtgccacattgtcttaatccagtctatcattgatggacattagggttaattccaagtctttgctattgtgaatagtgtcagttcatgtcctttgtagggacatggatgcagctggaaaccatcattctcagcaaactatcacaaggacagaaaaccaaacaccacttgttctcactcataggtgggaattgaacaatgagaacacatggacacaggaaggggaacatcacacacaggggcctgtcgtggggtggggggagcagggagtgatagcattaggagatatacctaatgtaaatgactagttaatgggtgcagcataccaacatgacacatgtgtacatatgtaacaaacctgcacgttgtgcacatgtatcctagaacttaaagtataataataataattatatatatatatatatattagaaaaacaaacaaaaagaatcttctgtatcctgagcaaggagttctgaaggcacaggtgtgtgtagagtgtggatttgtctgtgtggctgttgtgggaggggaaacaggagggggctgcttcttattcttggaggcctctgtgcagaggtgacatgggaggtgggtgtagggggctggagagagaggagaccttgattgtcctgggtccttagagatgcagggaagggaagaataaggtgtgtgtggttggggtgaaggtttaggggaggagagctgaggagtgtggaaggtttgggataatgtgaggaggccagttccagactgtccctggcacacacccttcatgtaatctctgaaataaaagtgtgtgctgtttgtttgtaaaagcattagattaatttctaggggaattgaggagacctctgaggcacctctgaagcttctttaggtctaaatttcttgctagtttttgtttttttagggtgtatatttttacatagcagaagtgactgtgaaactaactttttgaattaaagttttaacacagttactattttattataatgctaatagttttctagtagttacatcttattcttttgtatataatagttgtgacacaacttacctcactttcccctttttgttgacctttattatgactttcaccaaaagttgaaaatgtatgtttctggttaatttttactttatatttttcatttgtaattcttttgaattattttgacctatttattggccaattataattactgctgtaagaattccctatttcatttggtagggaatgtacaatgatctactgtctaatatcttaagggcttagtatttttctcagtgactttgtgggttctttgtactgtaagattattaacactttattgatatttgattcagcattttctccagtttgtggtttgtatgtggattttgaaaagtattttccatgttaagaatttgaacatttttatttaataaaatatattgcgaaatttgtattaatgatttacaatcatcttaaatccaccattttgtggtattattgtctccaggtttctccttacttctaaaaaaaaactgtatttattgagagtatgctagtgtcagggattttcctgggcataagcaccccaagtgacgagtcccagacactgccttaatccaaatgtcattctggaaagaaaaatcattttacaatgataggcctaataataattaagcttgtgttgcacaggagatacattgatcagctaaatgtaaacgtaagaactttcaaaactaaaatgacattccttaatcattctctctgcgttaggactcatgcttttctaggaacgtaaaaatttgagaatcatttctgtctgtcccaccttcccaggggcagaaccatttctgtggtgttctaaggtgtgagtgcatggtggtagtattcccaaaaattcatactcggtttcctcatgtacccaactctgtccctttatctacccacattgctttaagtcatatttttctgtcacggtgtacaaggatgataaataggtgccaagtggagcacccaagtgtgatgagccccctcacagtggaatggagtgacaagctttctaacctcataaattgaaggctatcttcagtcattgttttatatattttacgtgcattaatcctcatataaccccaagaggtaaattagtataattatccttcattgtaggtgacaaagttgagacacagaagaatcaaaaaactcttccagcatcaaccagtaaaaggcagaccttggatttcaaccaggcaacatggctcagatatcagttttaattactacactctactgtcaaagatttgtaaacactttgacaatgcatgccaatttcaagctatgaagaaacaaacacaatttttcacaacatctctcaaatctaatgggtcctcactatcaagattaaattccaggctgatgacactgtaaggccacatggccagctgtgctggaggcctggtcaaggtcagagcctgggtttgcagagaagcagacaaacagccaaacaaggagacttactctgtcttcatgactcattcccactaccttttttttctcctagtccaacctaaggtgactgtgtatccttcaaagacccagcccctgcagcaccacaacctcctggtctgctctgtgaatggtttctatccaggcagcattgaagtcaggtggttccggaacggccaggaagagaaggctggggtggtgtccacaggcctgatccagaatggagactggaccttccagaccctggtgatgctggaaacagttcctcggagtggagaggtttacacctgccaagtggagcatccaagcatgatgagccctctcacagtgcaatggagttagcagctttctgacttcctaaatttctcacccaccaagaaggggactgtgctaatccctgagtgtcaggtttctcctctcccacatcctattttcatttgctccatgttctcatctccatcagcacaggtcactgggctactgggggtagccctgtaggtgtttctagaaacacctgtaccccctggggaatcagtcacgcctgccaggcgggagaggctgtccctcttttgaacctccccatgatgtcccagttcagggtcacccactctccccaggctccaggccctgcctctgggtctgagactgcgtttctggtgctgttgatctgagttgtttgttgtgagaagaggagaactctaggggccttcctgacatgaggggagtccaatctcaactctgccttttattagctctgtcactctagacaaactacttaacctcactgagactcaggctttctgttgatcagattttgaagttgcgccttacatcaaggctgtaatatttgaaatgagtttgatgcctggaccttgtaactgttacagtgtgatttgaaaaccgttttttcccccagaaatggctagttattttagttcttacagggcagccttctttcccattttgaaagctctgaatcttagggtctcaattacagaggttcaatttggaataaacactaaacctggcttcctctctcaggtgcacggtctgaatctgcacagagcaagatgctgagtggagtcgggggctttgtgctgggcctgctcttccttgggacagggctgttcatctacttcaggaatcagaaaggtgaggagcttttgggagctgaatctccataggcttttctggaggaggaactatgactttgctagggttagttctcagtatatcagtggccctggataaagcctttctttccccaaatgacctccaatgtcctgataatccagaaatcatcagtgcatggttactatgtcaaagcataatagcttgtggcctgcagagataagagaaaggttaacaagtagggattctttggttggagatcctggagcaaattaaggaagagccactaaggctaatgcaattacgctggatcctgtgacagacacctcatgcttcatgggtcacatggtctgtttctgttcctctctgccctggttggtgtgggttgtggtgttagagaaatctcaggtgggagatctggggctgggacattgtgttagaagacagatttgcttccatatcttttaagtgtatatcttttcctctttttcccaggacactctggacttcagccaacaggtaataccttttaatcctctttcagaaagagattcagtttccctagaatgatggcagaggtgataaggcatgagacagaaataataggaaagactttggatccagatttctgatcaggcaatttaccccagaactcctcctctccacttagaaaaggcctgtgctttgcaggatcattggctcagggagacttaggaacttgtttttcttctccctgcagtgctctcatctgagtcgttgaaagcggggaaaagaagttttagtaaagccaggtctgaaaacaatttcctctgtctctgcaggactcttgagctgaagtgcagatgaccacattcaaggaagaaccttctgccccagctttgcaagatgaaaagctttcccacttggctcttattcttccacaagagctttgtcaggaccaggttgttactggttcagcaactctgcagaaaatgtcctcccttgtggcttccttagctcctgttcttggcctgaagcctcacagctttgatggcagtgcctcatcttcaacttttgtgcttccctttacctaa

SEQ ID NO: 34 - DNA, 4422 bp, synthetic construct
  misc_feature 1..4422: note = Description of sequence: Gene: HLA-DRB5
  source 1..4422: mol_type = other DNA
cacgtttcttgcagcaggataagtatgagcgtcatttcttcaacgggacggagcgggtgcggttcctgcacagagacatctataaccaagaggaggacttgcgcttcgacagcgacgtgggggagtaccgggcggtgacggagctggggcggcctgacgctgagtactggaacagccagaaggacttcctggaagacaggcgcgccgcggtggacacctactgcagacacaactacggggttggtgagagcttcacagtgcagcggcgaggtgagcatggtggggggcggggcctgggtccttgtgagctgggaatctgagtgtgtgtgtgtgtgtgtgtgtgtgtgtgtgtgtgtgagagagagagagagaggaagagagagacagagagggagcgcgccatctgtgagcatttagaatcctctcaatcttgagcaaggacttctgaggacacaggtgtgtgtgtagagtgtggatttgtctttgtggcctttgtgggaggggaagcaggagggggcttcttcttatccttggaggcctctgtggggaggtgacatgggaggtgggtgcagggggctggagagagaggagaccttggttgtctcgtgtccttagagatgcagggaaggaaagtgtaaggtgtgtgtggttggggtgaaggtttaggggaggagagctgaggggtgtggaagatttgtgataatatgaggaggccagttccagactgtccctggcacccacccttcacgcagtctctgaaataaaagtgtgtggtgtttgtttgcaaaagcattagattaatttctaggggaattgaggagacctctgaggcacttctgaagcttctttaggtctaaatttcttgctattttttctgtttgttttcttagtgtgtatatttttacatagttgaaatgactgtgaaactaactttttgattaaaattttaatacactgttactattttattataatgctaatagttttctactacttacctattattcttttatatataatagttgtgacacaacttatctcactttcccctttgttgacctttattatgacattcaccaaaagttgaaaatatatgtttctggttattatttaatttatttttttatttggaattcttttgaattatcttgacctatttattgcccaattataattactgctctaagaattccctattgtatttggtaggtaatggacaatgatctattttctgttatctccagggcttagtatttttctcagtgattttgtgggttctttgtactgtaagattattaacactttattgatatttgattccacattttctccagtttttgatttgtatgttgattttgaaaattctatgttaagaatttgaacatttctgtttaataaaatatattccaaaacttttattaatggtttacaaaccatcttaaatctatcattttgtggtatttttgtctccaggtttctccttccttcttaaaaaaaatgtatttattgagagtctgctagtgttagggattttcctaggcataagcaccccaagtaatgagtcccagaccctgcctgccttgatccaaatgtcattctggaaagaaaaattattttacaatgatagtataataatagttatgcttgtttgcatgggagatgcattgatcagctaaatgtaaatgtaagaattttcaaaactaaaataactttccttaatccttctctctgctttatgactcatgctttgctgggaacttaaagattggagaaccatttctgtctgtcctaccttcccaggggcacaaccatttctgtgttgttctaaggtgtgagtgcatggcagtagtattcctaaacattcatactcagtctccttttgtaccctactcggtccctttatctatccacattactttaaatcatatttttctctcaagctgtacaaggatgataaataggtggcaagtggaacacccaagtgtgatgagccctctcacagtggaatggagtgagaagctttatgacctcataaattaaaggctatcttcagtcattgttttatatattttatgtgcattaatcctcatataaccccaagaggtaaattactataattatcctctattatgggtgagaaagttgagacacagaagaatcgaaaaactcttccagcatcaaccagtaaaaggcagaccttggatttgagccaggcaacctggctcaggtatcagttttaattactacactctgtactttcaaagacttgtaaacactttgacaatgcatcccaatttcaagtgatgaagaaacaaacacaatttttcacatctctcaaatctgatgagcccccactataaagactaaattccaggctgatgacactgtgaggcctcatggccagctgtgctggaggcctggtcaaggccagagcctgggtttacagagaagcagacaaacagccaaaaagggagattcactctgtcttcctgagtcattccctctacatttcctttctcctagttgagcctaaggtgactgtgtatcctgcaaggacccagaccctgcagcaccacaacctcctggtctgctctgtgaatggtttctatccaggcagcattgaagtcaggtggttccggaacagccaggaagagaaggctggggtggtgtccacaggcctgattcagaatggagactggaccttccagaccctggtgatgctggaaacagttcctcgaagtggagaggtttacacctgccaagtggagcacccaagcgtgacgagccctctcacagtggaatggagtgagcagctttctgacttcataaatttctcaaccaccaagaagcaaactttactaatccctgagtgtcaggtttctcatctcccacatcctattttcatttgctccatgttctcatctccattagcacaggtcactgggggtagccctgtatagtttctagaaacacctgtatcctctggggaagcagtcattcctggcaggaaggagaggctgtccctgttttgaacctccccatgatgtcacaggtcagggtcaccccctctcccagggctccagaccctgcctctgggtctgagactgtgtttctggtgctgttgatctgagttatttgttgtgatctgggaagaggagaagtgtaggggccttcctgacatgaggggagtccaatatcagctctgccttttattagctctgtcactctagacaaactatttaacctcattgagtctcaggctttctgtttatcagatgttgaagccgtgccttacatcagggctgtaatattagaatgaatttgatccctgaaacttgtaactgttcagtgtgatttgaaaaccttttttttctccagaaatggctagttattttagttcttacagagcagccttcttccccattttcaaagctatgaatattgcagggtctcaattaaaaaggttcaatttgggataaaaatcactaaacctggtttcctctcccaggagcacagtctgaatctgcacagagcaagatgctgagtggagtcgggggctttgtgctgggcctgctcttccttggggccgggctattcatctacttcaagaatcagaaaggtgaggagcctttggtagcggctctctccatagacttttccagaggaggaaatagggctttgctgaggttagttctcagtatatgagtggccctggataaagcctttctttccacaaatgacctccaatgccctgctaatccagaaatcattaatgcatggttactatgtcaaaagcataatagcttgtggcctgcagagataagagaaaggttaactagttaagggtcctttggtttgaaatcctggagcaaattaaggaagagccactaaggctaatgcaattacactggatcctgtgacagacatgtcatgcttcatgggtcacatggtctgtttctgctcctctctgccctggttggtgtgggttgtggtgttagagaaatctcaggtgggagatctgggactaggacattgtgttgggagaatagatttgcttccataccttttaagtgtatatcttttcctctttttcccagggcactctggacttcacccaacaggtaataccttttaatcctcttttagaaacagattcagttttcctagaatgatggcagaggtgataaggcatgagacagaaatagcaggaaagactttggatccaaattcctgatcaggcaatttataccaaaactcctcctctccacttaggcctgtgctctgcaggagtattggttcagggagacttaggaacttgtttttcttcttcctgcagtgctctcatctgagtccttgaaagagaggaaaagaagctgttagtggaaccaggtctgaaaacaacactttcctctctctctgcag

SEQ ID NO: 35 - DNA, 4656 bp, synthetic construct
  misc_feature 1..4656: note = Description of sequence: Gene: HLA-DRB5
  source 1..4656: mol_type = other DNA
taagtatgagtgtcatttcttcaacgggacggagcgggtgcggttcctgcacagaggcatctataaccaagaggagaacgtgcgcttcgacagcgacgtgggggagtaccgggcggtgacggagctggggcggcctgacgctgagtactggaacagccagaaggacatcctggagcaggcgcgggccgcggtggacacctactgcagacacaactacggggctgtggagagcttcacagtgcagcggcgaggtgagcatggtggggggcggggcctgggtccttgtgagctgggaatctgagtgtgtgtgtgtgtgtgtgtgtgtgtgtgtgagagagagagagagagagagaggaagagagagacagagagggagcgcgccatctgtgagcatttagaatcctctcaatcttgagcaaggacttctgaggacacaggtgtgtgtgtagagtgtggatttgtctttgtggcctttgtgggaggggaagcaggagggggcttcttcttatccttggaggcctctgtggggaggtgacatgggaggtgggtgcagggggctggagagagaggagaccttggttgtctcgtgtccttagagatgcagggaaggaaagtgtaaggtgtgtgtggttggggtgaaggtttaggggaggagagctgaggggtgtggaagatttgtgataatatgaggaggccagttccagactgtccctggcacccacccttcacgcagtctctgaaataaaagggtgtggtgtttgtttgcaaaagcattagattaatttctaggggaattgaagagacctctgaggcacctctgaagcttctttaggtctaaatttcttgctattttttctgtttgttttcttagtgtgtatatttttacatagttgaaatgactgtgaaactaactttttgattaaaattttaatacactgttactattttattataatgctaatagttttctactacttacctattattcttttatatataatagttgtgacacaacttatctcactttcccctttgttgacctttattatgacattcaccaaaagttgaaaatatatgtttctggttaatatttaatttatttttttatttggaattcttttgaattatcttgacctatttattgcccaattataattactgctctaagaattccctattgtatttggtaggtaatggacaatgatctattttctgttatctccagggcttagtatttttctcagtgattttgtgggttctttgtactgtaagattattaacactttattgatatttgattccacattttctccagtttttgatttgtatgttgattttgaaaattctatgttaagaatttgaacatttctgtttaataaaatatattccaaaacttttattaatggtttacaaaccatcttaaatctatcattttgtggtatttttgtctccaggtttctccttccttcttaaaaaaaatgtatttattgagagtctgctagtgttagggattttcctaggcataagcaccccaagtaatgagtcccagaccctgcctgccttgatccaaatgtcattctggaaagaaaaattattttacaatgatagtataataatagttatgcttgtttgcatgggagatgcattgatcagctaaatgtaaatgtaagaattttcaaaactaaaataactttccttaatccttctctctgctttatgactcatgctttcctgggaacttaaagattggagaaccatttctgtctgtcctaccttcccaggggcacaaccatttctgtggtgttctaaggtgtgagtgcatggcagtagtattcctaaacattcatactcagtctccttttgtaccctactcggtccctttatctatccacattactttaaatcatatttttctctcaagctgtacaaggatgataaataggtggcaagtggaacacccaagtgtgatgagccctctcacagtggaatggagtgagaagctttatgacctcataaattaaaggctatcttcagtcattgttttatatattttacgtgcattaatcctcatataaccccaagaggtaaattactataattatcctctattatgggtgagaaagttgagacacagaagaattgaaaaactcttccagcatcaaccagtaaaaggcagaccttggatttgagccaggcaacctggctcaggtatcagttttaattactacactctgtactttcaaagacttgtaaacactttgacaatgcatcccaatttcaagtgatgaagaaacaaacacaatttttcacatctctcaaatctgatgagcccccactataaagactaaattccaggctgatgacactgtgaggcctcatggccagctgtgctggaggcctggtcaaggccagagcctgggtttacagagaagcagacaaacagccaaaaagggagattcactctgtcttcctgagtcattccctctacatttcctttctcctagttgagcctaaggtgactgtgtatcctgcaaggacccagaccctgcagcaccacaacctcctggtctgctctgtgaatggtttctatccaggcagcattgaagtcaggtggttccggaacggccaggaagagaaggctggggtggtgtccacaggcctgattcagaatggagactggaccttccagattctggtgatgctggaaacagttcctcggagtggagaggtttacacctgccaagtggagcacccaagcgtgacgagccctctcacagtggaatggagtgagcagctttctgacttcataaatttctcaaccaccaagaagcaaactttactaatccctgagtgtcaggtttctcatctcccacatcctattttcatttgctccatgttctcatctccgttagcacaggtcactgggggtagccctgtatagtttctagaaacacctgtatcctctggggaagcagtcattcctggcaggaaggagaggctgtccctgttttgaacctccccatgatgtcacaggtcagggtcaccccctctcccagggctccagaccctgcctctgggtctgagactgtgtttctggtgctgttgatctgagttatttgttgtgatctgggaagaggagaagtgtaggggccttcctgacatgaggggagtccaatctcagctctgccttttattagttctgtcactctagacaaactatttaacctcattgagtctcaggctttctgtttatcagatgttgaagccgtgccttacatcagggctgtaatattagaatgaatttgatccctgaaacttgtaactgttcagtgtgatttgaaaaccttttttttctccagaaatggctagttattttagttcttacagagcagccttcttccccattttcaaagctatgaatattgcagggtctcaattaaaaaggttcaatttgggataaaaatcactaaacctggcttcctctcccaggagcacagtctgaatctgcacagagcaagatgctgagtggaatcgggggctttgtgctgggcctgctcttccttggggccgggctattcatctacttcaagaatcagaaaggtgaggagcctttggtagcggctctctccatagacttttccagaggaggaaatagggctttgctgaggttagttctcagtatatgagtggccctggataaagcctttctttccacaaatgacctccaatgccctgctaatccagaaatcattaatgcatggttactatgtcaaaagcataatagcttgtggcctgcagagataagagaaaggttaactagttaagggtcctttggtttgaaatcctggagcaaattaaggaagagccactaaggctaatgcaattacactggatcctgtgacagacatgtcatgcttcatgggtcacatggtctgtttctgctcctctctgccctggttggtgtgggttgtggtgttagagaaatctcaggtgggagatctgggactaggacattgtgttgggagaatagatttgcttccataccttttaagtgtatatcttttcctctttttcccagggcactctggacttcacccaacaggtaataccttttaatcctcttttagaaacagattcagttttcctagaatgatggcagaggtgataaggcatgagacagaaatagcaggaaagactttggatccaaattcctgatcaggcaatttataccaaaactcctcctctccacttaggcctgtgctctgcaggagtattggttcagggagacttaggaacttgtttttcttcttcctgcagtgctctcatctgagtccttgaaagagaggaaaagaagctgttagtggaaccaggtctgaaaacaacactttcctctctctctgcaggactcgtgagctgaagtgcagatgaccacattcaagggggaaccttctgccccagctttgcatgatgaaaagctttcctgcttggctcttattcttccacaagagaggactttctcaggccctggttgctaccggttcagcaactctgcagaaaatgtccatccttgtggcttcctcagctcctgcccttggcctgaagtcccagcattgatggcagtgcctcatcttcaactttagtgctcccctttaccta

SEQ ID NO: 36 - DNA, 32767 bp, synthetic construct
  misc_feature 1..32767: note = Description of sequence: Gene: CIITA
  source 1..32767: mol_type = other DNA
attcaccatatccgtttgttagccccactaagcaaggctgtttcaaattgactcagcatttcttatttgtcaatttctaccccactcctcacccggggacttctgaaaacagtgaaggcttcaaagtaatcaccttacaaagaagggcatcatcctcatttcacacatggggaaactgaggaccacagagaggaagtaaagaggccaaggtcacacacctgctactaagttccccgtccagtgctctttcagcaaaaatgcatgaggcacacaagtcatgtttccaaaccttcatttcagtaccaccttcatcatttttgtgttttccacataacactttactattatttatgacagggttttttttaaccactcaccttttttacttatctttcttttctttttctttaggagaggcaggatctcactctgttgcccaggctggagtgtatttcatgatcatagctcactgcagcctccaactcctgggcacactcgatcctcccacttcagcctccagagtagctgggactatagttgtgcaccatcatacatggctaatttttaaaaaatcatttgtagaaaaattagctggatgtaggagaatggcgtgaacccaggaggcggagcttgcagtgagccaagatagcgccactgcagtccagcctgggcgaaagagcgcgactccgtctcaaaaaaagaaaaaaagaaaagaaaagaaaaattagctggacatagtggcaggtgcctgtaatcccagctgctcgggaggctgaggcaggagaatcagttgaacccagaacccgggaggcggaggttgcagcgagccaagatcatgccattgcactctagcctggcaataagagtgaaactccgtctaaaaaaaaacaaaaacaaacaaaaaaaaccccaacaatttgtagacatagggtgtcactatgttgcccaggctagcctccaactcccggcttcaagcaatcctcctgcttcggcctcccaaaatgttggaattacaggcacaagccacctggcccagccatctactttatattcaaataaaactttacgtcccattataaagggaaaaaatggcaaaaacaggaggtaaccatttaacaagaaagcagagtgatgttagattatagcaagatactgttgactgtagaaggctctgaggctagagagctgctttctataaaacggagtgatcatatattagaagaggtgttaaagacatgttcacaccaagctgagacttcctccttgataccaccaggaggatgggcagagactggaaaagacactaactttctccctatgggagtcagtattatttagcatcactttggcgggtcaccccaaaccatctgactacaagggtaccatatttgggttaacactcttttggtataatttatgttttagtccaatgtcttgggatgaaaatgacaggtgggccacttatgatctccagagaaattcagggcaatttggtgtgggagtaggcatggtagaggagagcagcatctaagaagtccccagcagaggctctcagcttgtcttgaggcatctgggcggagggctatgatactggccccatcctgcagaaggtggcagatattggcagctggcaccagtgcggttccattgtgatcatcatttctgaacgtcagactgttgaaggttcccccaacagactttctgtgcaactttctgtcttcaccaaattcagtccacagtaaggaagtgaaattaatttcagaggtgtggggagggcttaagggagtgtggtaaaattagagggtgttcagaaacagaaatctgaccgcttggggccaccttgcagggagagtttttttgatgatccctcacttgtttctttgcatgttggcttagcttggcgggctcccaactggtgactggttagtgatgaggctagtgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcagagccccaaggtaaaaaggccgggaaagcatcttaatttagcgtgcagtctcagctggtcctgccattccagataaacagagaaaccattctgaattggggatgggggtgaggatgggaacaggagtctgtgtcctgctggggcaggccattggaagatgtgaaagagttgtctatttccttccaccggagggagacttcaggtcagccaggtgtctggagtatgaaccatgtatcagcaccgaaaggttctagaagtcagactttcgggcagtgtgtcactaactctcagcatgctggcctggctcggcccacagcaaggtcttctcgcctccctttgggtaaatactgaggggtgcctctgcaggacgggacctctgccagactccactccatacccagagaagcagggaaaccaaaattggagtcagccttgaggtgtagctgttgagccctcagcagctggggagagctggcggatgctgccctccccccagtttcctaatggtgttgtttaaaaagggtcaggggacgggggaacagatggtgggaagagcacagtgcagacacctggcaccggctctgaaggcagcatggcagctacaccgttggctgggaagggtgtgcccctgaagaagtcgtttacattctcgagtcaattttcctggagtgtacaatggacctgtgggaaagcctgtatgaaagggtaatgatgagggacctagcacagtgtccaatattttataggaactggaattgagctcataggagctcaattttattggcattgctgttgttggatggttaaaggggtggtatcccttttctcagactcccctgaaatgtatggtttgctttgaacccagagactgatgacaggtctgccggtgtggttgggtgcagccttaagttgctacgggaaagtgttggagggggagaagtcagaggtaaccttgccccctccctcaattccagatgaggaaattcaggcctgaaaagggaaagtgaccacctcaaagtctcatgccttggaggacccagcaggaatccaagacctctgaaaaggaccggcagggctcttgccacggctgggggtgtggtcatggtaacacaggttttccatccatggaaggtacctgagggattttctcttcctccctagggccagcatcagaggagtgaatagctcagttagctcatctcaggggccatgtgccctcggaggtggtttgccactttcacggttggactgagttggagagaaacagagacccacccaggggtggggacaagctccctgcaactcaggacttgcagatcacttgcccaagtggctccctagctcctggctcctggcccggggcctgggactctccccgaagtggggctggccactgtgaggaaccgactggaggcagggacctcttggatgccccaggcagttgggatgccacttctgataaagcacgtggtggccacagtaggtgcttggttgctccacagcctggcccgagctcagcgctgcagaaagaaagtgaaagggaaaaagaactgcggggaggcggggaggtaggatgaccagcggacgagctgccacagacttgccgcggccccagagctggcgggagggagaggccaccagcagcgcgcgcgggagcccggggaacagcggtaggtgaccaaagtctcctctgtaacccctaaggtcgggctgagaatcgaggctccgagactgtcagctacttgctcaaggtcacacagcaagtctgggaggatggggggatggaatatgcaaaatgtagggccgggaaacacctcgtttccagcatccccgcaacgactctgcgcgggaaccaggagccgggaacccggagcttggcttgctgtgcccagagctccggggccgtgggcgggtggcaggaaagcctggcggcagcttctgcagagaagccggagcgcagactgggagcgcggagcagacacactcccccggccacccttggccgactccgcgcgcccgggatcctgcagaggtgcgcgcccttcttgtacgccagactttggaccagggccgccgttccctgagcttcactttccctgttgggtcatattccatctctaactctggaatcttgggtattgggctctccaggcggggggccctgctcagggaggcagtagggagccaaacctttaaccagaggatgggataagtcctcaactctcgttgaacatcttggcgaaggtgtgtgttgttgggaggggtgggggagggatccccccggactgaaccgatctcttgatctctcacttctctacctcgctttggggccctgagtcacaccctctaaggagagaggctaaagcgccccggaaagccagcgtgcgaatgccggggtgggagtgggagattggatctccctggggtccaggaaagccggaatcggagccaccatgcttagcttagtctggaactcttaaaagccgcggtcctcctgagtcccacagcccctctccaccctaggtggcacaggagaggtggcaaaagcctagaagttcaaggcatggctccctccccagccgcagcctggagtgtctaactttggcaggaagtcttccgtttctgctccccactccagagaaaaaataaataaatacttctccggagtgagattaaggaaacaggtacttcttcctcttggagaaagaggagccaaaggaacttgactccaacaaatgatcaccttgcaaacccccggctcccttaggggatgacctggtctccaacaatctcagagcgtttggaggcagggtctttggagatgactgagtggggaatcccaggctccccacacatgaacatcacctgggatgatcaacctgttcaggatgtaggttcccgggctcacccccaggcccggttggctaggcctggggtgaggctgagatcctgcaggttaaaccatctatcccaggtgactccaatgttcgtttgtggggcaaaagtccctcaagtcagagacactgggaggcgctgatgtggtctcatctctttactctctccctgttacaaaacctctatcagaaaaggagtaccaggaggtgttttgttttgttttgcttaacgccacatagcaagcagtttgcagacgcaggatttgaaccctggtctactgagagcccagcccagtgcagcaagcagatgtgaacctccacaaatgcagggcaggtcccaggcagtcagagagaaacctgttgatacccagtctgtctgatggctggggcttggtttcatgtcctaagacccagcctcagataccttgagaagagcagaatcagaaattaacccaaggaaactctggattagttccaggcaatcagggaagtggctggagggaggtaggaaacttctggacttagcctcagatttgccttgagtcacactgacaagtgacttcccctctctgaggttcatctgtaaaatggataaaacatgagagcttgcttgatgatgtctgatgtgtacagaaaaagcactttagaaaaccaccaaagaatattcatgcattgggaataattattatagatcctaaagggattgtttagaaagatcagagaggggccgggagcagtggttcacacctgtaatcctagcactttgggaggccgaggcgggtggattgcttgaggccaggagttcaagaccagcctgggcaacctggagaaaccccaactctactaaaacacttttttgtgttttagccgggcatggtggtgcacacctgtaatctcaactacttgggaggctgaggcacgagaatcacttgaacccaagaggcggagtttacccgagaggtggaggttacccgagagctgagattgcaccactgcacgccagcctgggcaacagagcaagactgtctcaggaaaaaaaaaagagagagaaaaaagtcagagaggaaagcataggcagatgggaggtttccaaagaccaagacacacacagtcaaaagacagttcagaaaggggcattagttaccaccgattctcataggaaggacatagatagttttcagattccttcaatccttctgacatcaaggacaaagtctctgattttccctgatgtttgacagtgccctaatgcttgctaagatttctttttaacaaagtggtaaagaagctgagattattttagtaacattgtttctttttcaaccacaagtgatactgttggtttggttttgcttttccgtttatacatggaaaaaaaagttttctttttgtaaaatcatggtaaaatacatataacataaaatttaccattttaaccactttcaaatgtacatttcagtggcattaaatatttcaaactgttgtgcaacctccgctaccatccatctccagaactttttcatcctcccaaactgaaactctacccaataagcaataactccccactgccccttccacccagctcgtggtaactccaacttcctgtctctatgttttgggctactctagacaccgcctataaatagaattacgtgatatttgtctttttgtgcctggcttctttcacttagcataatgtcttccaggttcatccatgtcgtgccatgtgtaattcctttctatggcggaatagtattctaatatgtggatatcccatattttgttcatccattcatttgttgatagacacttgggttgttcctacctcttgactattgtgaataatcttgctaggtacatgggtggacaaatatctgtctgactccccgctttcaactctagaaagctttcttttaaaatcggtctatttaagtttctttaaaaagttaatttaagaaaaatactaaggtaaattgtagaacaggtgatatgtcaatatgaccccaaaaattgtaaagatggaagacgaatgcctgcagtttgggaaaccggatgagaggaaagacctggaagctattgtaaaatctatgctgagggtctggtgactactagaccaagggcatggcagcagagggcagctgaaagacttcaggaaggaaagatataagaattaagaaaggggcctggtgcagtggctcatgcctgtaattccatcaccttgggaggtcaaggcaggatgatggcttgagaccaggagttcaagaccagcctgggcaacagagcaagacctttttttaggaaaaaaatatataaattaataaaaataaatttttgaaaatacagattccaggccgggcatggtggctcacacctgtaaccccagcactttgggaggctgaggccggtggatcacctgggatcaggagttcgagaccagcctgggcaacatggtgaaacaccgtctctcctaaaaatacaaaattagccgggtgtagtggtgcatgcctgtaatcccagctactcggtaggctggtggaggagaatcgcctgaacccaggaggcggagattgcggtgagccgagatcacaccattgcactccagcctgggcaacaagagtgaaactccgtctcaaaaaaaatgaaagaaaatacagattcctgggcccggctgcagactccctgaatagaatcactgggggtgaggcccaggacgctgtgtttttaacacccttcccaggtaattctcactcacagccaagcctgccgatggctggactccgtagtcagatctcagcaagcaggactggctcgtggttcatcttggggtctccagcacccagcagagtgctggcggggggactgtgttaagtagagccggatgggtcaggactggagtgtggggcctggctttgctaactgcagtcacaagatcctcatcactccaccccctaccccagcaccagggtcaggttcccttccaggaaaaacttgttggactcctttccaggtcattgactgcagttaggagtggcatgcagaagggaggctatctggagctgggacagaccaggcaagcggggaagggaactcttatcccaaccctgcattctggacagggaccgaaggtctcctggctgcagctcatacagctcatcttcattctcatcctgggaccaccaggcccctcacccacaataactttaccctttactggcaaatgcacaagctggagatgatggcccattcgagttttaagattttccaagttgttatgaggtcagtgtcctcaatcgggacccagagaagataggtggcttgcctgaactttcagagggggcatctgtggtgtcttttgtggggtcctgtaggatttccacgtccctcttggaggaaaaatagaagtaagttgtctcaggtcagattgtctgggaagccaagtaggagatggagatttgcatataggaggtttactggggacattcacaggagggaacacccgaaggaaaagggaagaaagcagggttgggaagcagcaggagttgaggtgcaatgcagtcacaacagaggtcttagccaatcccatagggagcactcaagctgggacagcaaggaagctagaccttcacatccctcatctacctccgtcctcccatacaggcatagcctcccccatgatgaacactcagcaccagactgatacatttgttataatcaatgaacctacattgatgcatcattattacccaaggtccatggtttgtttacatgagggtttactcttggtgttgtacattctgtgggtttggacagatacatgatgtaatgtatatgacattatagtatcacacagactcatttcacttgtgactgagcttgtccttctttctttaacctgcctccaactgctgagaagccctttggctagggattgcagccaattttaatttgttttccactttcctgctctgaagttactttgtttgccagaggctggttggttcaaacccaaagggcagggagctgcaatggaaatccattcaacggtgctggaattgggatggcaaaaaggagcagcaggcaggtgtcagcagggagcagctctttgcatcccacagttcccagggagcttaccattctttcattcattcattcatcctttcatttatgagacaaagtctcactctgttgcccaggttggagtgcagtggcacagtcattgctcactgcagcctcaagctcctgagctcaagccatcctcctgcctcagcctcccaagtagccgagactacaagggcactccaccacacccagctaatttttgtatcttttatttctgaagagatggggtttcactatgttgcccaggctggtctcaaactcctggcctcaagtcatcctccctcctcagcctcccaaagtgctgggattacaggtgtgatctcagacagcttacctcttaatcacctttccatctgagctggcagcagagctgggaaagtgcacccaacaccccagatgctgcaactttagtgcttcctgctccttctctgtgtggaactggcctgtttgggtacccagggccagtcccctccaagaccctctgtagaaaaagaccctactgtacacctcaattccattgcaggacaggacataattgaatcctgcctctccctgccccagtcacccatccctcccaagccattatcttgggacctgcaagggcctccaagccaagggtggcaaatccaaataccaaaggggccataatgatgtaaaatgtgtccaggcagaaggcaatagggagtagtggggcctctggtaacaattgtgtgtactctatatagagacattgtgatggtgatttagacacacacacacttatattggcttgggaaacaaaacaaataggcctatcgactggattcaacttttgattttgcagccaagcccctgtggcacagctgaggagcttgcaggtcagaaaggggatttagagccaaccaatagcaatatctccagacctctcctgacacgaagctcatctgacctcctgcccccattacacacatacacatgcacacacatgcatgcaggcacatgcacgcacgtcttctagtctccctgccttgaggacaaatgatcagatcctgcaactgtttccaggcccataagcccctgagcattgcagtgttgctacatgccttagtttcctctccgtctcctgtgacctcctcgtgccactccaggaagtgaaagtcttgagtgagaccagtagtttctcttaagtcctctctctgtctcgttctcctgggatatctacacttggtttgcaaacagcaggctggagagtgaggggcttcttcctctacatccagctcctcccccatcctgctccaagtccagcatgttcctatctcttccccaggaccctgggctcttgggcacaaatcaagccatcaccagacagcctgtggggtggcaagagcagaggtttgtttctttgtgaacatctataggtaccaggctctgtgcaggccctattgttggtgagccatagaggtaccaacctcaccccatgaatcctcctttgtttcttttcttttcaacccctgggcctttaaaccttcgattaactgcccagtgcagcccaggtcatggtacactatgatctttggttcacatgagggttttctcttcattaatgtccctatttcccatactgggtgcccattctaatgcatttcaggtgtatctttctgtttgtatgtgctcttataaaacatgttttgccttttttttttttttttagatggagtctcactctgtcacccaggctgagttttctatttttagtagagatggggttttaccaccttggccaggctggtctcaaactcctgacctcaggtgatccacccgcctcggcctcccaaagtgctggaattacaggtgcgagtcaccacgcctggccacagtttcttacttttgcattctgtactatgtttttaggatctacttgccttgcttctgatgcatttgatctttgcttctaatcacggacattgttctctatggagtgcaggggttacttttcacctgtccacacccccagtgatggacacctaggtgacctccaactctctgtcaccacaaataacacagcagtaaccatcctcagatgggttctcctggggagttgtgggaacatttcttcaggatatatagacccaagagctgaattgccaggtggtaggatctacatatccttagatattctacatgatgccaggtcgttctccagagcggccccatccatctacattcccacaaacccgtgtaaccaacacttggcatttcccagatgtctaacttttgccagttgtacagtatcaagtgtcactttgctgttttatttttggagtttacatcttagtaccacttactcgctaagtcactttaggtaagtcacaacaacccagtgacataagcactgtgccaacaaaatgcagtcgattctgtgactcagagaggttgagtaacatttgcaaagtcacacagcaagtgtggaactaagatttgaacccagagcctgcacccttaaccactatgctattattatgttttgtctctctcctaaaatatcatctgggtctacattgcaagcctctttggcagggactgtgtggtaaacctctttggggccagtaaggaactgagctcagtgactttcttacggggtgctcagtgaatcattgttttacagctttagaggggaaaagtgagagatgtttcttagagtttatctggaggactacagggtgcatgagcaaaagcggaactcatttgagctactacccccacaaagaagctgtttggggtctggtcatggtagataggcattcctaggcccctggcttcccaacaccatttggcccccggaagcagcttgtcatgaggttggggagcaggttggtgttggggggggccttgggggaaaggaaaaggagaagactgccaggcaacgaggaatcagaatctcaaaaagactcaattgaaggggctccaacactcgttgaaacggaggctcagagggctaaggtaatgtactcaggatcaaaggcagagcagaaactcaaaccatgtctctaggcacacagttcagaggtcttcccattattcctcaggaggagaagttcatggtcaagagtagggaagtattccttctcaagctttttttttttttgtctgagacagagtcttgctctgtcaccgaggctggagtgcagtggcgtgatctcggctcactgcaacctccgcctcccaggttcaagcaattttcctgcctcagcctcccaagtagctgggattataggcacccaccaccacgcccagctcatttttgtatttttgcagagacggggtttcaccatgctggtcaggctggtcttgaactcctgacctcaagtaatccgccagcctcggcctctcaaagtgctaggattataggcgtgagccaccacccctggcctccttctcatgctctttaatcacagttcagagcagtgctggaggcttttaggggtagcaaaagagccttcttctccctttcagtgcctccttagaccctcaaagatagactccccgcctctgggaagaaaacatctaatgaactggacgtaatctcagcgcctgcgtctgtaaaatggctataataacaacaatctcccagagttgccgttaacagttgaaacagaatagtaaatgcaaggcatctagcacagccccagacacagagaaatctcccctggtcactctcacagaggtaagaagcacattaataataaaaatggtcatcacgcatattgtactcaccatgcagaagttgtgctgccttaagctactgttcattgagcacttactggtcgccagtcccctttctaagggctccatgcaacttaactcccctaatcctatgagttgccaattattatcccccactttacagataaggaaactgaggctccaagtggtttaaaaaatgaacttacccagattgtagtgcgttggtaggtagggataacctgaaagttgggaaatttcaccccaaagctcaccatctgagctccattcatctcccaccagctgagatggaacgttatgctgttcccagaagagatgcatgcactgatcttcagatagtttaagtctgagttagaacattgtggcgacacctgatttccctcgggggagagagaggtgaattgttatttcagacacctgtttagtgtctacacttagcctttcaaatcctgcatgcagtgtgaagtggggatgaaaataaaccccaaaaaggacttttgatggcaaaaagatcaggaatattttctcgagttggatgtggaaggtggtggcccagggggaagcacagtttcaaagaggaccttctaaaaatactgcccatttaccaaaaagacacagaccgcgcctctgccctgcccccacatggctcctcaaaaggagaaattgtcttgccacccacgccttccctcgccggggtgctggggggccaacagcaattcaaaaaggctatagagtgggctgagactttggagatgggtagtttggttcaaatctcagcccactaatccctctgagcctgtctcttcatctgaaaaacaggaatacaaatagctgatgtgtaagttagaccaaatggggtaatgcctacgagtcatgtcatttggtacctggcacttgctggacactgtgaattctagttctatatcattgccacggatgaagagaggctgccatgcgcagaaacatgctgagagctgggagaaggtgagctagagggatacagtccctgccctcagggagagctgaagggctctctctcctgccacgggatgatcagggccatcttttccctccatcccgctctggcaatgtggtttggagaaacagtccaatgaaggggcctttgaagcacttcctaagcatctatagacataggaaacttggctctggggatagaggaaggtcccatgccaagattcaagtagatcttggtgtgactgggaggcagctgtggcgttgtggggaaacctagagtcttgggcctgaatttcagctttgacaccctctaatgtaatgggcttcctttgactgtaggcttcccacatagctggcaccccacacagaactcccttggccttctgatgatcctgcaggacaatcttttttgttttttgttttttgttttttttttgagatggtatctcactttgtcgcccaggctggagtgcagtggcgcagtcttggttcactgcagcctctgcctcccaggttcaagcgattctcctgcctcagcctcccaagtagctgggattacagaagcacgccaccatgcctggctaattttgtatttttagtagagacgggatttcaccatgttgcccgggctggtctcaaactcctgacctcaagtgatctgcctgcctcggcctcccacagtggtgggctgcaaggcaatcttcattctccagaactaatattgggaaaactgatgttcagagaggtcaagtgacttggctaaggcatcacagccagaattcagatgcaagctcctaaaccacatgaccttggacaagccatttcactgaggcgtgtgaacctcctctaagctcaaatgggcataacactctaccctgccttttccccagagctttttgggaactcagatggtgtggaggatggaggcagaagtccactgtgagctgcaaatccttgatcacccaagaggaagtgcttaaggtgcaggcttcagtcttagggaagcaggaagtggggcgctgaaatgtgtattgggggcttctgggagggggtttcttcttgtccaggttgagccaatgacgcagatcttaggccctctggtgactttttgcccaggcaacttctcagtggtggtgatggggagcgtggccagaccatccaagctgccctgctggctgtgggggccttttttttttttctgagacacagtctggctctatcacccaggctggagtgcagtggtgcaatctcagttcactgcagccttgacacctccccaggctcaagctatcctcccacctcagcctcctgagtagctggttctacagatgcacatcaccacgcccagctaagttttgtgttatttgtagagatagggcttcaccatgttgcccaggctggtctcaaactcctggactgaggtgatactcctgccttggactcccaaagtgctggaatttctacagatgtgagccaccatgcccggcctatggggcccttcttgttcctatgaagtctgcagggtccccttatgcaaaagaacagaaagctcggcttcacaaagtttgacctcggaagaaatggaagggaaagggtccctttgtcactctcacaaacgtaagaagcaaactaataatgatggccatcacacatattatactcaccatgtagaagctgtgctgccttaaacttctgttcattcagcacttactgattgccagtcccattgctaagggctccgcgtgacttaactcccctaatccttgtgagttgccaattattattcgcattttatagatatggaaactgaggctccaagtggtttaaaaaatgaacttgcccaggttacagtgtgttggtaggtaggggtagtctcaaagttggtaaattttacgccaaagctcacgatacttgccttctttcccttctcaatttatttctctgactgtgtgcttaaccacacagcagtgtcatccagagtttgaagcagagctcatcaataagtgggggtgggctaaatgtctccaggctcctccctgcggtccctcccaccatcccccatctgtgataccacctgtgtctctatgcttgagcccagacaggacaagggtgatcatttcattgggaacattttgactggggggaaagagcaccagcctgggagtcttggcagggggagtgggggagaaggggcaggggcagccaccgacaggctatgaggtcttgtgcaagtcacgtccttcccggggtctcagtcttgtcatctgcagagtgaaggaaagaggtcaagcttgaaggtccctggcagacccaatgccctgtgagaaaactgagtttgggtaacttattattttatagatatggaattatcttatagaaatgttatagatatggaatcattttatagatatggcccaaagtccaaagtcacacaactcattcacggagttgctaggatcagtctggagctcttgtcacgagatcacactgcctcaaagaggaggcagattgctgcaatgtaggttcttaataaagagcatgggtggaatgggaggcgggcatcaggcaggagctggggcaaaggtgtcttttctcactgctctgagagaacagattttgtgcctgtgtccctgaccaaggggctgggagaggtggagcatctcacccccaaccatctcccagctttggccctgctccaggaaaagggcggtggccccatctttcagttctgcttcctctagtcatggttggctgggatctcgctgcaagccggttatgtttgggttcctgcagcccccgctcaaccgtcatgaacaagtgggcccagccagtgccccagcttggactggaaagatcattctctgtgacatccgggggtctgtgctgagcctctccccaggcagtgaaagctgtggatgcgtgaaagaaaatgggtctctgggccgggtgtggtggctcacacctgtaatcccagcactttgggaggccaaggcgggtggatcacctgaggtcgggagttcgaaaccagcctgaccaacatggtgaaaccccatctttactaaaaataaaaaaattagccaggcatggtggcgggtgcctgtaatgccagctacttgggaggttgaagcaggagaattgcttgaatccgggaggcagaggttgcagtgagccaagattgcaccactgcactccagcctgggcgacagagtgagactctgtctcaaaaaaaaaaagaaaaagaaaatgagtctcaggaatggggtggcaagttgagtgtgtgttttctcatgagcttactcttcaatgtattctcttctgggcccagaacagggcagtaggagcctccctctggtcttccagcttcagggccagctctcctcttacctcctccaggaagctccctctgtttctttccagccttcatctgcatgtcatgtgtggtgggttgaaaggtggccccccaaaagatatgtccatgtcaaatccctggaacctgtcagtgtgactttatttgggaaaagggtctttgcagatagaattaagttaaggggctgggtgcagtggctcatgcctgtaatcccagcactttgggaagctgagattggaggatcacctgaggtcaggagttcaagaccagcgtggccaacatggcaaaaccccatttctactaaaaatacaaaaattagctggtcatggtggtgagcacctgtaatcccagctactcaggagactggggcaggagaatcgcttgaactgggaggtggaggttgcagtgagccgatacagcgccactgcattccagcctgggcaacagagcaagcctctgtctcaaaagaaggcaaaaaaagaattaaagatcttgaaatgacatcatcctggattatctaggtgggtcctaaatccaatgacaagtgtctttataagagaaagagacttgctacatgaggagtaggtgatgtggccactaaagctgagtttagagtgatgcagccacaagaccacaagtcaaggatcaccagtagttcccagcagctagaggaggcaaggaagcattctcccctcgagcctccagaggcagcgtggccctgctgacaccttggcttcaacttctggcctccagaaccatgagagaataaagtgctgttgttctgagctacctggatgctggtaccttgtttcagcagccctgggaaatgaatataccatggaaacatggacctgaaccacggatcctggcaggagccttagagccacacagatgagccttcagcttggctttccccagcaagagctacatgatcttgaggaagctgcttgtcttctctgggtctcaagttcttcatctgccaatggagacaataatgcttacttcctaggttttaacagctttattgagatgtaattcacatatacaatttgcccacttaaaaagtacagttcatgcctgtaatcccaacactttgggaggccgaggagacaggcggatcacttgaggtcaggagtttgagaacagcctggccaaaatggtgaaacactgtctctactaaaaatgcaaaaattagccgggtgtggtggtgggtgcctgtaatcccagctgctcaggaggctgaggcagaagaatggcttgaacctgggaggcagacgttgcagtgagccaagattgcacccctgcactccagcctgggtgacagagactccgtcttaaaaaaaaaaaaaaaaaaaaaaaaaaaaaagtagagttcagtgttcttttgggaatactcagagagttgtgtgaccatctcccatccccacagtcaattttagcacatttcatcaccccctaaagaaactccagacccatcagcagttgttccccatttccttccaaaatccctctctgccaagccctaggcaaccactaatatgctttctttcttaaaaaaaaattaatagagataggcttttgctatgttgcccaggctggtcttgaacacctgggctcaagcgattctcctgtctcagcctcccaaagtgctgggatttcaggcatgagccaccacgccccacccctcctactttatgtctccatggatttgcctgttttggtcatttcaaataaatagaatcatacaatatgtgaccctttgtgtccggcttcttcacttagcattttttttaaggttcatccatgttccagcatgtatcagtgctttctccctttaatggtggaatactttattgtatagataagcaacattttgtttatccatttattttttggtggatatttgggctgtttttatattttggctattttgaattatccttctgtgaacattcgtgtataggtttttgtgtggatatatctggtttttcttgttttcttttgttttattaatacataatatgatacatatttatggggtatatgtgaatatttattacatgcatagaaggtataatgatcatgtcaggatatttgaggtatccacatttgggattgtttaaagattaaatgaaatagtgttaaaagtatttaatatgcccttcaacaaatgatgaggaaatcttagaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgtgccaccatggagttggggcccctagaaggtggctacctggagcttcttaacagcgatgctgaccccctgtgcctctaccacttctatgaccagatggacctggctggagaagaagagattgagctctactcaggtgggccctcctccctctggtctcttccggtatcccccacccctcagcttgctgtagagacggcaatcaggggaaattctggtccctgtcctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtccatacattatcatctggaatccttacaacaacaaaggaaatacatattattagtttctgtcttttacaaatgaggaaattgaggtttagagaggtgaaacgacctacccaagtttgcattagagatagtaatgtacagatctgaaattctaacctaggtctttctcgtttcaaagtctccatttttaaccacaaaaaaagaaagtgtctggggttggaagatttgctaaggcttaagcagagaagctaatttgtagccttaagaagagctacagctttcagacaagggcaggactcttggggagaggtggtggtggtgttgctgctagtggtttgttttttgggggagagacgtgggatagagaatctcttctcaccagcatcaccaccacctcatacagagcctatgttgaaatagaaaaaagtggcctttttctggaggatgcagcccctaccaggaagtaactgcttggctagcaaagtgcaagccggagtcctgcccccattttccttgttggcttgggcacctgtgtgcagtgaacaacctgcaaaactcttcttagtggtcttcttcctgccaacccaggaacagcagcattcagcagttctgtgcttgatccaaatgtggccaatttttccccagagcacagtgtattgaaaatcataccaaggaagaaaatggaggggactagtaattgttcatgtttcagaaattacagaactgagagcttgactgcttccttcccttcctggggatttacatgggggaagcagaagtgattggggctgaggacattcacatttcctcacaactggaggcagtcattaaggatttgggcatgtcataggatttgggtgtttttctccccccttagtggcatctccacaggcagcgaaagctgtctaagcaaataggtgaggcagtgcgttagtccattttttgttgcttaaaacaacatacctgacactgagtaatttataaagaaaaaaaatatttcttacagttatggaggctgagaagtccaaggttaggggctgcatctggtgagggccttcttactggtgaggactctctgcagagtcccaaggcagtgtggggcatcacgaggcaagaggtttcggtgtgatagtctctcttcctcttctcataaaaccactagtcttactcccacgacaacccattaacttattaacctgtttatccactaatccatgaacggattaacccattcatcaaggaaaagccccagtcacctcttaaaggccccacctctcaatactgccacataggggattacatttatacatgagttttggagaagacaaacattcacgtcataacaggcagcttcacccaagtctgctcctcaagtctgtgggttttctctctgcttctggaaggtgaagcccaggcctgggcaagtattcttttgcagcatcctgaaatatgaccttagcttctaaatgtgtaaaatggagataataatcacacccaccatcacgggatggttgtaagactgaataagttacaatgtgtaaagcatttagaaaaggaatttgcacagagtaagcattcagtagatgttaggtactaccccacaaccaccgttatcattattatcaccatcattattatcatcttcattttcatcaccttcattatggccatcattccattcctcagctcagaagccttctatggctccccattgcttataaaatcaagtcctaactttccccagcatgaaacttctaccaccgtcacctatctctcagggtttcctaaacatactctgaacaagtttcctcactctgccactgtgacccagaaagctatctgttctccttctcccagactctgcctcatctttcaaggcctggttccaggatcccttcctccaggaagccttccctggttgccctattccaccatacaccttttttctcggacttcatgtcatggtgcctatattccaagggctctgagccatgtacccctttatatagttaccactatttactgagtgcctactgtataccagctactgtgttggatgctctagatgtagaacctctaattatcaccatcgattcctgggtagtaggcattatttattcatcttacacagatgagaaaatggaggcccacagtggttaaataactagcccaagattgcacagctagtagggctagtggaaagtagaggtggaatttgaactcaaatcctgcagtaactctaccattctgccttgctcttctttgtagcagtagataagttttcatggatacatacctcatccttttgattagattaagggcccctggagtgtcagtgttcattcatttgtttgatcattcattcattcaacaaacatttcttgagtccccactgtgtgccaggcccagaggttccccagcccaaggcctggcacacagtgggccttcagttagaccttgttgattgactgcgcttttccttgtctgggcagcggaactggaccagtatgtcttccaggactcccagctggagggcctgagcaaggacattttcagtaagtttgtggtgggtggggaggtcttggctcagcctgcatttcctgccttgttccctggggggtgccctaatacctgacgaccattcattgatgggcagtcagacccctctccccaaggtggggacaatagagactcaccttgggctttcattgattgtgtgagttggtctctggtttttctcaaagtagagcacataggaccagatgaagtgatcggtgagagtatggagatgccagcagaagttgggcagaaaagtcagaaaagacgtgagtgagcccctccctgatccaacctagccttgcttgagacctggcctttccttgactccaaagcctgctgtgggtccaacttgcttccctcgctaagtcctgtctggttgggaggccctttaaaagccaacaggagccttaaaatgtacatctgattatttcatggccctgataaccctccaatggctacaaaatacatgccacaaggcctgtatggcccttcctccctctccaaacccactatgagacaccacacttcagccaccaccagcttctccactcctataacccatgtgccctttcaagcctcggggcctttgcagttgctatagtctctatctggaatgcccttcccccagttcttcccatggctgactcctttgaatctttctggtgttggctaaactgtcacctcttcctggaacccttctctgaccatccttccatgtagattagctcagttattctcaccttgtgtgtctttttccttgcagtttagcactcattaccatctggacatattttacgccttgctctcccactgtgaggacagggaccttgtctttcttgctcgtgactgtttccccagcatctagtgcagtgcctggtatgcagtagcacctcagtagatatctgttgaatgaaaacatctgtaaaatgggtgtaacagttaactgagtacttattatgggtctgaccatgtgtaagtcctgtatctatttattcagttcttaaacaggtgaatcgcacacagggtatgagattttaaaagtgcaaagaatattcagtgaaggctgggcgcagcggctcacacctgtaatcccagcagtttgggaggccaagggggacggatcacttgaggtcaggagtttgatacctgcctggccaacatggtgaaaccgcgtctctaccaaaaaatacaaaaattagccgggtgtggtggtgcacgcctgtaatcccagctactcgggaggctgaggcaggagaatcgcttgaacccaggaggtggaggttgcagtgagctaagatcacgccactgcactccagcctgggtggcagagtaagactctgtctcaaacaacaacaaccaaaagaatattcagtgaaaaaaaaatctccctcccactcctgttctgtagtccctcagttttgctcctatcttagtcaacctatgtgaccagcttcctttttgccttgtcagaaataaagcgagctcctgtatttgctttaattttccatgcaaatgaccacattttatttacactattctgcattttgctttcttcatttacttcattactccctgtctgggaggtcacgttccatattattgagggagaggacacggtcctatcttcagcagcttctttattttctttcttttcttgtttttcttctttatgttttcatttgataaatatttccccccaagattttattgtgaaagttttcggccaggcgtggtggctcacatctgtaatcccagcactttgggaggccaaggctggcagatcacctgaggtcaagagtttgagaccagcctggccaacatggtgaaaccccatctctactaaaaatacaaaaattagccaggcgtggtggcgaacacctgtaatcccagctaccagggaggctagggtgggagaatcgcttgaacctaagaagcagaggttgcagtgagccaagattgcgccagtgcactccagcctgggcaacagagctagactctgtttcaaaaaaaaaaaaaaatgttttcaagatacaataaaagtgaaagaatggtactatgaaggtccatatacccaccatgtaccattttgattctacaattaatgttgggttttattcatcttatcacatctctattcatctgtccatccctctatccatccacacacccattcattatatttttggtacatttcaatgtacatttcaaatgtaaattgcagatttacactcccctcgtgtacttcagcatcctttcttttaaggccatgcagtgtttcgttgggtagatgaaccataatttgtttaagcagtcccttgctaagggacacgtgggtttttaatctgttgctgtgagtgcttgatactcactcactgatttctggcatactcctgcaatgtaggaaccaccacccccatttcatagatgtgagatcaaaggttcagagaagctgcattgcaaaggcacacagctaggaagcggttcaaaagcaggttcctttgttttgttttgttttgttttttggctcaaacccatgttgtgtccccatttgtgactcggcctcttctgctgctacactgcctggtatggtttgagatggggtttgggttcaagccaaggagctggggttggaatgttagagcgaggggaggaaaatggacccccaagaccactacccagccttgaagttaaggccgtatagcctgctagagtcctgagccccttctggcttgggacatcctctccctggggcagctgatcacatgttttctctgcagccttcccagaggagcttccggcagacctgaagcactggaagccaggtgtgcagggcaggtgggctggggttgggaagggtggatgccttggggaggggatggaagagattgaactcctggcccaagtctgatggggatggtgcatggtgcagcccctgcccttctttgggtagaggctgagagcttggggtcccttagagtcctctaaggggctcacgactgtttgtggaaggtggtaggggcttggagctaacagattgttcataggttctattctgccccagctctccgtgtggaggccctagggtcctgctcagcatagctctcagagccaagtcacaaggagaggactgggggactgcctggcacagagcagttgctgatcaacacagctgcagccagggctgagaagatgacaagcatttcctctgtcaggagagacatccatgccactccagggccctccccatcccaggaaggcccctccaagcacccagtctctaacacagcccacttcctcacagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccagtatgttagggggcttggagagagtgggctttctccctcttgggaggtggataaggagttgacactaaggcagggactgtcaggaactggacctcacctctccccaaccctatcagtgtcactcacctctgccccagccagttttatccttggggcccccgtcagctcaatccctggcagagaacatgcctcagaaggaaacacatccgtgcgagcatcaggagggaatctccaccaattagagcattcacctgccagccttattcgttcatcagctcttcatggagccccttctctgtgccaactgctctgctaggtgctaagaattccatcagggcaggacagaaacagctactgctcttagggaaattagggccctttaggggggtcagacattaatcaaataagcaaaagcagaatcgcaaacacaggtgctatgcaagatcccacctcactgcctttgtctcttgcagtgcctttctccagttcctcgttgagctgcctgaatctccctgagggacccatccagtttgtccccaccatctccactctgccccatgggctctggcaaatctctgaggctggaacaggggtctccagtatattcatctaccatggtgagtgcggggcctggctccccgaccacctctccctcctacctgactgctccctgacctcatcctccccatactccatgcacttggcagtggtgccctagcaccttctcatgatcctccctccccagcacaactttctctgtccctatttcccaaaaatcaaagcagaagagcagagattcctgaggtggtctgggtgcatgaccaagagtggttgtgtggccttccatatgttgctttccccttctgacccttagtttttccttattttttttaaatgtttttgtttttaaattgcaacatcaatacatgctcactaataagctaatgacaagtgatgtttacaagaattcactgtctctctcactcttttagtatactgatgtgaatagtttggtgtgcatccttgcatagctttctctgatttgtcttgtttcctaatcagtaaaatggtccctactgacccaccacactgatgggttgtgacttgtctcagggaaggtgtgtttgggctgtcagggatgagaccaggaggatgcttccactcaagtccagagcatccattcctggttgcaagacagtttcagtataacgcactggtatctcagaagcagagaagcagttcctgatatctaggaaaatgagcactgaccacacagcaggaagagtgcaatagtagtacctgccctaagttccttatgagctaattgcacccatttcacagattggaaaagcaaggttccaggagctagatgtaactccagagctctttcttcagctctgtcctgaaatatcttcctttggaagttctactctcttctctccagacttcctgagctccacagcccagtttggagtaggggtgacccaagtgcatttctggaatcaatacctggttattctcacaccactctccacccccaatgtaggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacaggtaaggacccttagggcctgtgagaggtactagaagcaggatcgaggccctggggaaggaattgtctcaaataagatccacaagcaaagctgcctgtagggacaacaggtcatgtttaggggtcagtcgggacagggagggtctccagggagtggaggcatggttgtgggggaatggacagctagagaatccacatcccatccaaatacagccatcagccttcagctgcttgttacccagcagaaaagccagacaatgtggtcagatcttttgaagttgaaaaccctgatttttacatagatattgtgattttttaatttttattttattatttatttattgttgagatggagtctcactctgttgcccaggctagagtgcagtggtgcgatctcagatcactgcaacctccgccctacaggttcaagtgattctcctgcctcagcctccggagcagctggaattacaggtgtgcaccgtcacacccagccaatttttgtaatttcagtagagacaagtttaaccatgttggccaggctggtctcgaactcctgatctcaagtgatccacctgcctcgacctcccaaagtgctgggattacaggtgtgagccactgcacccagcctgatattgcgattttcttttttaaacatgagtgaaatttttaaaagtacagttggccaaagaaagacatatttgagttagatacagcctcaggccgctatcttataacctccatcttgggaaactcagcccatgaaggctgtaaggactaagtggcccagagggaggggggtccccaggggtaaccctcaccctaaatctggcacctgcttctccatctccagagcacaagacgtcccccacccaatgcccggcagctggagaggtctccaacaagcttccaaaatggcctggtgagtgatgcgggatctctctgccctgggtggtggagatggaagcccatatctggcttcacattgctcattcacttgacacttattcaaccccttctttgttggctcacacactcatttatttattcattcattcattcattcacttatttgattatgccatcatttcaccattcactccaccactccctcaaataatatttattgcccttcaataagcctgcactgggtgttaacacttcattgtcaggctgagagaatgaatgaggggcaagcgaatgatgaagtgagtgagtggatgaagtgagaagttgatgaggatgggagcaatggaaagtgggaataaaccagtgaataagagaattactaaattactaaatgcttactctcacatactggtctaagcatgttaggtatgctaaatcattaacatctcacacaatcccaatattatccccattttccagatgaggctactgaggcatggtgggatgtatagcatgctcagaattgctccagcaagtggtagaaccgggatttgaacccagacacgtgggctccactgtcttaactcttaacactccctcccatgacctccccatggtgagtgagtagtgatcaagcgaagaaaaagcttagtgagtaaatgaaggaatgagaaaagatggccatgcatggtgccttatgcctgtaatcccagcactttgggaggccaaggcaggtggatcacctgaggtcaggagttcgagaccagcctggccaacatagtgaaaccccgtctccattaaaaaatacaaaaattagccagacgtggtagtgggtgcctgtaatcccagctacttggcaggccgaggcaggagaatcgcctaaacccaggaggtggaggttgccgtgagctgagattgcgccagtgcactccagcctggggcacagggcaggatccatctcaaaaaaaaaaaagagagaaaaggtctaaaaggagtggacacttaaacaagagagacgatgagtgaaggaatgggtgtggaaatgagtgaactaatgaatgagtggtgggtgactgaatgaagcaaatgatgaagactgtatgggggccagatgtggtggctcacacctgtaatcccagcactttgggaggccaaggcggaacgattgcttgagcccaggagctcaagaccagcctgggcaaaatagtgagaacttatctccacaaaaaaacaaaaaagaaaaattagccaggcatggcagtgggcgcctgtagtcccagctactcaggaggctgaggtgggaggattgcttgagcctgtgaggttgaagctgtcgtggaccatgattgtgccattgcacgccagcctgggcgacatagtgagaccccatctctacaaaaaaatcaaaaaattatctgggcatggtgtcacatgtctgtggtcccagctacttgggaggtggagtcaggaggatcgcatgagcccaggaggttgaggttgcagcgagctgtgatcacaccactgcattccagcctgggcaaaaaagccagaccctgtctcaaaacaaaacaaaacaaacaaacaaaaaaactgtgtggggaacagatgtaaatgatggtggcagtgctggccttgtggtggctggccctggccctgcctctcacatacccccaccctgacacgcccctggcctttgcagagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacggcttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccctactcgggccatcggcggctgcctcggtggacaggaagcagaaggtgcttgcgaggtacctgaagcggctgcagccggggacactgcgggcgcggcagctgctggagctgctgcactgcgcccacgaggccgaggaggctggaatttggcagcacgtggtacaggagctccccggccgcctctcttttctgggcacccgcctcacgcctcctgatgcacatgtactgggcaagg

(19) United States
(12) Patent Application Publication
(10) Pub. No.: US 2026/0355388 A1
(43) Pub. Date: Nov. 12, 2026

(54) ENGINEERED CELLS AND IMPLANTABLE ELEMENTS FOR TREATMENT OF DISEASE

(71) Applicant: SIGILON THERAPEUTICS, INC., Cambridge, MA (US)

(72) Inventor: Mandar Bawadekar, Bedford, MA (US)

(21) Appl. No.: 19/120,522

(22) PCT Filed: Oct. 11, 2023
(86) PCT No.: PCT/US2023/034936

§ 371 Date: Apr. 11, 2025

Related U.S. Application Data
(60) Provisional application No. 63/415,271, filed on Oct. 11, 2022.

(51) Int. Cl. A61L 27/38; A61L 27/20; A61L 27/22; A61L 27/54; C08L 5/04; C12N 5/079

(52) U.S. Cl. CPC A61L 27/3813; A61L 27/20; A61L 27/227; A61L 27/54; C08L 5/04; C12N 5/0621; C12N 2510/00

(57) ABSTRACT

Described herein are engineered mammalian cells comprising a reduction in the level or function of a major histocompatibility complex (MHC) class I protein complex component, and, optionally, a MHC class II protein complex component and/or CIIT A, as well as and methods of making and using the same.

### CLAIM OF PRIORITY

The present application claims priority to U.S. Provisional Patent Application No. 63/415,271, filed Oct. 11, 2022.

### BACKGROUND

Treating chronic and genetic diseases by implanting cells engineered to produce a therapeutic substance capable of treating such diseases has exciting potential to improve the health of patients with such diseases. To fully achieve the potential of such therapies, the implanted cells must be capable of producing therapeutic levels of the desired therapeutic substance for several weeks, months or even longer without overstimulation of the host immune response.

### SUMMARY

Described herein are engineered mammalian cells comprising a reduced level or reduced function of a major histocompatibility complex (MHC) class I protein complex, as well as related devices (e.g., implantable elements), compositions, and methods of making and use thereof. In an embodiment, the engineered mammalian cell comprises a reduced level or reduced function of one or more of a protein selected from human leukocyte antigen (HLA) A, HLA-B, HLA-C, and beta-2-microglobulin (beta-2M). The reduced level or reduced function of the MHC class I protein complex may be due to a mutation in a component of the MHC class I protein complex or may be due to the silencing or knock down of a component of the MHC class I protein complex.

In one aspect, the present disclosure may also feature engineered mammalian cells further comprising a reduced level or reduced function of a MHC class II protein complex. In an embodiment, the engineered mammalian cell comprises a reduced level or reduced function of one or more of a protein selected from HLA-DP, HLA-DM, HLA-DOA, HLA-DOB, HLA-DQ, and HLA-DR. The reduced level or reduced function of the MHC class II protein complex may be due to a mutation in a component of the MHC class II protein complex or may be due to the silencing or knock down of a component of the MHC class II protein complex. In another aspect, the engineered mammalian cells described herein may also comprise a reduced level or reduced function of a class II major histocompatibility complex transactivator (CIITA).

In another aspect, the present disclosure features an implantable element comprising an engineered mammalian cell described herein, or a plurality of engineered mammalian cells. The engineered mammalian cells may comprise an embryonic stem cell (ESC) or an induced pluripotent stem cell (iPSC). The engineered mammalian cell may comprise a retinal pigment epithelial (RPE) cell, a CCD-33Lu cell, a MRC-5 cell, a MRC-9 cell, a MCF10a cell, or a cell derived therefrom. In an embodiment, the engineered mammalian cell comprises an engineered retinal pigment epithelial (RPE) cell (e.g., an engineered ARPE-19 cell), or is derived from a retinal pigment epithelial (RPE) cell (e.g., ARPE-19 cell). In an embodiment, implantable element comprises at least one cell-containing compartment which comprises the engineered mammalian cell or plurality of engineered mammalian cells described herein. In an embodiment, implantable element comprises one cell-containing compartment comprising the engineered mammalian cell or plurality of engineered mammalian cells described herein, and a second compartment surrounding the cell-containing compartment. In an embodiment, the implantable element further comprises at least one means for mitigating the foreign body response (FBR) when the implantable element is implanted into the subject (e.g., a compound of Formula (I) as described herein). In an embodiment, the implantable element comprises a polymer selected from alginate, hyaluronate, and chitosan. In an embodiment, the implantable element comprises a cell-containing compartment surrounded by a barrier compartment comprising an alginate hydrogel and optionally a compound of Formula (I) (e.g., a compound of Formula (I) described herein) disposed on the outer surface of the barrier compartment. In an embodiment, the implantable element is formulated for implantation into a subject (e.g., into the intraperitoneal (IP) space, the peritoneal cavity, the omentum, the lesser sac, the subcutaneous fat). In an embodiment, the implantable element is configured to shield the engineered mammalian cell or plurality of engineered mammalian cells from the recipient's immune system and mitigate the foreign body response (FBR) (as defined herein) to the implanted device. In an embodiment, the implantable element is capable of delivering a therapeutic agent (e.g., a protein) for a sustained time period (e.g., one to several months up to one to several years) after implant into a subject.

In another aspect, the present disclosure features a method of treating a disease or disorder in a subject, the method comprising administering to the subject an implantable element comprising an engineered mammalian cell described herein, or a plurality of engineered mammalian cells described herein, wherein the engineered mammalian cells or the plurality of engineered mammalian cells comprise a reduced level or reduced function of a MHC class I protein complex. In an embodiment, the engineered mammalian cells or the plurality of engineered mammalian cells further comprises a reduced level or reduced function of a MHC class II protein complex and/or a reduced level or reduced function of a CIITA. In an embodiment, the disease or disorder is a lysosomal storage disease. In an embodiment, the disease or disorder is a metabolic disease.

In an embodiment, an implantable element described herein, or a plurality of implantable elements described herein, is combined with a pharmaceutically acceptable excipient to prepare an implantable element preparation or a composition which may be administered to a subject (e.g., into the intraperitoneal cavity) in need of treatment with a therapeutic agent produced by the device. In an embodiment, the subject is a human, the engineered mammalian cells are derived from a human cell (e.g., an RPE cell, an ARPE-19 cell) and the implantable element preparation or composition is capable of continuously delivering an effective amount of a therapeutic agent to the subject for a sustained time period, e.g., at least any of 3 months, 6 months, one year, two years or longer.

### BRIEF DESCRIPTION OF THE DRAWINGS

FIG. 1 is graph showing that Beta-2M protein expression in IDUA-expressing ARPE-19 cells containing beta-2M shRNA was considerably lower (89% lower) compared to that in IDUA-expressing ARPE-19 cells containing the scrambled control shRNA.

FIG. 2 is a graph showing that beta-2M expression levels were decreased 99% in ARPE-19 cells using CRISPR and a beta-2M-targeting gRNA, compared to ARPE-19 cells modified using the scrambled gRNA.

FIGS. 3A-D are a set of graphs showing that reduction of beta-2M protein expression (FIG. 3B) in ARPE-19 cells with reduced beta-2M protein expression (FIG. 3A) results in decreased HLA Class I expression (FIG. 3D), compared to beta-2M expression in wild type ARPE-19 cells (FIG. 3C).

### DETAILED DESCRIPTION

The present disclosure features mammalian cells (e.g., human RPE cells) engineered to modulate the level or function of a major histocompatibility complex (MHC) class I protein complex or a component thereof (e.g., beta-2-microglobulin (beta-2M)). In an embodiment, the mammalian cells are engineered to reduce the expression of the MHC class I protein complex or a component thereof, e.g., beta-2M. The mammalian cells may be engineered to produce a lower functioning or non-functional variant of the MHC class I protein complex or a component thereof (e.g., beta-2M), or the expression of a MHC class I protein complex or component thereof (e.g., beta-2M) may be silenced or knocked down or knocked out. The present disclosure also features mammalian cells further engineered to modulate the level or function of the MHC class II protein complex or a component thereof, and/or the level or function of a class II major histocompatibility complex transactivator (CIITA). In an embodiment, the mammalian cells are engineered to reduce the expression of the MHC class II protein complex or a component thereof, and/or the level or function of CIITA. The mammalian cells may be engineered to produce a lower functioning or non-functional variant of the MHC class II protein complex or a component thereof, and/or CIITA, or the expression of the MHC class II protein complex or a component thereof, and/or CIITA may be silenced or knocked down or knocked out.

### ABBREVIATIONS AND DEFINITIONS

Throughout the detailed description and examples of the disclosure the following abbreviations will be used.

| | |
|---|---|
| CM-Alg | chemically modified alginate |
| CM-LMW-Alg | chemically modified, low molecular weight alginate |
| CM-LMW-Alg-101 | low molecular weight alginate, chemically modified with |
| | Compound 101 shown in Table 4 |
| CM-HMW-Alg | chemically modified, high molecular weight alginate |
| CM-HMW-Alg-101 | high molecular weight alginate, chemically modified with |
| | Compound 101 shown in Table 4 |
| CM-MMW-Alg | chemically modified, medium molecular weight alginate |
| CM-MMW-Alg-101 | medium molecular weight alginate, chemically modified with |
| | Compound 101 shown in Table 4 |
| HMW-Alg | high molecular weight alginate |
| MMW-Alg | medium molecular weight alginate |
| U-Alg | unmodified alginate |
| U-HMW-Alg | unmodified high molecular weight alginate |
| U-LMW-Alg | unmodified low molecular weight alginate |
| U-MMW-Alg | unmodified medium molecular weight alginate |
| 70:30 CM-Alg:U-Alg | 70:30 mixture (V:V) of a chemically modified alginate and an |
| | unmodified alginate, e.g., as described in WO2020069429. |

So that the disclosure may be more readily understood, certain technical and scientific terms used herein are specifically defined below. Unless specifically defined elsewhere in this document, all other technical and scientific terms used herein have the meaning commonly understood by one of ordinary skill in the art to which this disclosure belongs.

As used herein, including the appended claims, the singular forms of words such as “a,” “an,” and “the,” include their corresponding plural references unless the context clearly dictates otherwise.

“About” or “approximately” when used herein to modify a numerically defined parameter (e.g., amount of a therapeutic agent secreted by an engineered cell, a physical description of a device (e.g., hydrogel capsule) such as diameter, sphericity, number of cells encapsulated therein, the number of devices in a preparation), means that the recited numerical value is within an acceptable functional range for the defined parameter as determined by one of ordinary skill in the art, which will depend in part on how the numerical value is measured or determined, e.g., the limitations of the measurement system, including the acceptable error range for that measurement system. For example, “about” can mean a range of 20% above and below the recited numerical value. As a non-limiting example, a hydrogel capsule defined as having a diameter of about 1.5 millimeters (mm) and encapsulating about 5 million (M) cells may have a diameter of 1.2 to 1.8 mm and may encapsulate 4 M to 6 M cells. As another non-limiting example, a preparation of about 100 devices (e.g., hydrogel capsules) includes preparations having 80 to 120 devices. In some embodiments, the term “about” means that the modified parameter may vary by as much as 15%, 10% or 5% above and below the stated numerical value for that parameter.

“Acquire” or “acquiring” as used herein, refers to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., using a fluorescence microscope to acquire fluorescence microscopy data.

“Administer,” “administering,” or “administration,” as used herein, refer to implanting, absorbing, ingesting, injecting, placing, or otherwise introducing into a subject, an entity described herein (e.g., a device or a preparation of devices), or providing such an entity to a subject for administration.

“Afibrotic”, as used herein, means a compound or material that mitigates the foreign body response (FBR). For example, the amount of FBR in a biological tissue that is induced by implant into that tissue of a device (e.g., hydrogel capsule) comprising an afibrotic compound (e.g., a hydrogel capsule comprising a polymer covalently modified with a compound listed in Table 4) is lower than the FBR induced by implantation of an afibrotic-null reference device, i.e., a device that lacks any afibrotic compound, but is of substantially the same composition (e.g., same cell type(s)) and structure (e.g., size, shape, no. of compartments). In an embodiment, the degree of the FBR is assessed by the immunological response in the tissue containing the implanted device (e.g., hydrogel capsule), which may include, for example, protein adsorption, macrophages, multinucleated foreign body giant cells, fibroblasts, and angiogenesis, using assays known in the art, e.g., as described in WO 2017/075630, or using one or more of the assays/methods described Vegas, A., et al., Nature Biotechnol (supra), (e.g., subcutaneous cathepsin measurement of implanted capsules, Masson's trichrome (MT), hematoxylin or eosin staining of tissue sections, quantification of collagen density, cellular staining and confocal microscopy for macrophages (CD68 or F4/80), myofibroblasts (alpha-muscle actin, SMA) or general cellular deposition, quantification of 79 RNA sequences of known inflammation factors and immune cell markers, or FACS analysis for macrophage and neutrophil cells on retrieved devices (e.g., capsules) after 14 days in the intraperitoneal space of a suitable test subject, e.g., an immunocompetent mouse. In an embodiment, the FBR is assessed by measuring the levels in the tissue containing the implant of one or more biomarkers of immune response, e.g., cathepsin, TNF-α, IL-13, IL-6, G-CSF, GM-CSF, IL-4, CCL2, or CCL4. In some embodiments, the FBR induced by a device of the invention (e.g., a hydrogel capsule comprising an afibrotic compound disposed on its outer surface), is at least about 80%, about 85%, about 90%, about 95%, about 99%, or about 100% lower than the FBR induced by an FBR-null reference device, e.g., a device that is substantially identical to the test or claimed device except for lacking the means for mitigating the FBR (e.g., a hydrogel capsule that does not comprise an afibrotic compound but is otherwise substantially identical to the claimed capsule. In some embodiments, the FBR (e.g., level of a biomarker(s)) is measured after about 30 minutes, about 1 hour, about 6 hours, about 12 hours, about 1 day, about 2 days, about 3 days, about 4 days, about 1 week, about 2 weeks, about 1 month, about 2 months, about 3 months, about 6 months, or longer.

“Cell,” as used herein, refers to an engineered cell or a cell that is not engineered. In an embodiment, a cell is an immortalized cell, or an engineered cell derived from an immortalized cell. In an embodiment, the cell is a live cell, e.g., is viable as measured by any technique described herein or known in the art.

“Cell-binding peptide (CBP)”, as used herein, means a linear or cyclic peptide that comprises an amino acid sequence that is derived from the cell binding domain of a ligand for a cell-adhesion molecule (CAM) (e.g., that mediates cell-matrix junctions or cell-cell junctions). In an embodiment, the CBP is any of the CBPs described in international patent publication WO2020069429. In an embodiment, the CBP is a linear peptide comprising RGD and is less than 6 amino acids in length. In an embodiment, the CBP is a linear peptide that consists essentially of RGD or RGDSP.

“CBP-polymer”, as used herein, means a polymer comprising at least one cell-binding peptide molecule covalently attached to the polymer via a linker. In an embodiment, the polymer in a CBP-polymer is a synthetic or naturally-occurring polysaccharide, e.g., an alginate, e.g., a sodium alginate. In an embodiment, the linker is an amino acid linker (i.e., consists essentially of a single amino acid, or a peptide of several identical or different amino acids), which is joined via a peptide bond to the N-terminus or C-terminus of the CBP. In an embodiment, the CBP-polymer is any of the CBP-alginates defined in WO2020069429.

“Cell-binding substance (CBS)”, as used herein, means any chemical, biological, or other type of substance (e.g., a small organic compound, a peptide, a polypeptide) that is capable of mimicking at least one activity of a ligand for a cell-adhesion molecule (CAM) or other cell-surface molecule that mediates cell-matrix junctions or cell-cell junctions or other receptor-mediated signaling. In an embodiment, when present in a polymer composition encapsulating live cells, the CBS is capable of forming a transient or permanent bond or contact with one or more of the cells. In an embodiment, the CBS facilitates interactions between two or more live cells encapsulated in the polymer composition. In an embodiment, the presence of a CBS in a polymer composition encapsulating a plurality of cells (e.g., live cells) is correlated with one or both of increased cell productivity (e.g., expression of a therapeutic agent) and increased cell viability when the encapsulated cells are implanted into a test subject, e.g., a mouse. In an embodiment, the CBS is physically attached to one or more polymer molecules in the polymer composition. In an embodiment, the CBS is a cell-binding peptide, as defined herein or in WO2020069429.

“Conservatively modified variants” or conservative substitution”, as used herein, refers to a variant of a reference peptide or polypeptide that is identical to the reference molecule, except for having one or more conservative amino acid substitutions in its amino acid sequence. In an embodiment, a conservatively modified variant consists of an amino acid sequence that is at least 70%, 80%, 85%, 90%, 95%, 97%, 98% or 99% identical to the reference amino acid sequence. A conservative amino acid substitution refers to substitution of an amino acid with an amino acid having similar characteristics (e.g., charge, side-chain size, hydrophobicity/hydrophilicity, backbone conformation and rigidity, etc.) and which has minimal impact on the biological activity of the resulting substituted peptide or polypeptide. Conservative substitution tables of functionally similar amino acids are well known in the art, and exemplary substitutions grouped by functional features are set forth in Table 1 below.

TABLE 1
Exemplary conservative amino acid substitution groups.

| Feature | Conservative Amino Group |
|---|---|
| Charge/Polarity | His, Arg, Lys |
| | Asp, Glu |
| | Cys, Thr, Ser, Gly, Asn, Gln, Tyr |
| | Ala, Pro, Met, Leu, Ile, Val, Phe, Trp |
| Hydrophobicity | Asp, Glu, Asn, Gln, Arg, Lys |
| | Cys, Ser, Thr, Pro, Gly, His, Tyr |
| | Ala, Met, Ile Leu, Val, Phe, Trp |
| Structural/Surface | Asp, Glu, Asn, Aln, His, Arg, Lys |
| Exposure | Cys, Ser, Tyr, Pro, Ala, Gly, Trp, Tyr |
| | Met, Ile, Leu, Val, Phe |
| Secondary Structure | Ala, Glu, Aln, His, Lys, Met, Leu, Arg |
| Propensity | Cys, Thr, Ile, Val, Phe, Tyr, Trp |
| | Ser, Gly, Pro, Asp, Asn |
| Evolutionary | Asp, Glu |
| Conservation | His, Lys, Arg |
| | Asn, Gln |
| | Ser, Thr |
| | Leu, Ile, Val |
| | Phe, Tyr, Trp |
| | Ala, Gly |
| | Met, Cys |

“Consists essentially of”, and variations such as “consist essentially of” or “consisting essentially of” as used throughout the specification and claims, indicate the inclusion of any recited elements or group of elements, and the optional inclusion of other elements, of similar or different nature than the recited elements, that do not materially change the basic or novel properties of the specified molecule, composition, device, or method. As a non-limiting example, a therapeutic protein agent secreted by an engineered mammalian cell described herein that consists essentially of a recited amino acid sequence may also include one or more amino acids, including substitutions in the recited amino acid sequence, of one or more amino acid residues, which do not materially affect the relevant biological activity of the therapeutic protein agent, respectively.

“Derived from”, as used herein with respect to a cell or cells, refers to cells obtained from tissue, cell lines, or cells, which optionally are then cultured, passaged, immortalized, differentiated and/or induced, etc. to produce the derived cell(s).

“Device”, and “implantable element” as used herein, refers to any implantable object (e.g., a particle, a hydrogel capsule, an implant, a medical device), which contains an engineered cell or cells (e.g., live cells) capable of expressing and secreting a therapeutic agent following implant of the device, and has a configuration that supports the viability of the cells by allowing cell nutrients to enter the device. The terms “device”, and “implantable element” are used herein interchangeably.

“Differential volume,” as used herein, refers to a volume of one compartment within a device described herein that excludes the space occupied by another compartment(s). For example, the differential volume of the second (e.g., outer) compartment in a 2-compartment device with inner and outer compartments, refers to a volume within the second compartment that excludes space occupied by the first (inner) compartment.

“Effective amount” as used herein refers to an amount of any of the following: engineered cells secreting a protein, a device preparation producing the protein, or a component of a device (e.g., amount of a therapeutic agent co-expressed with another therapeutic agent by cells in the device, number of engineered cells in the device, amount of a CBS and/or afibrotic compound in the device) that is sufficient to elicit a desired biological response. In some embodiments, the term “effective amount” refers to the amount of a component of the device (e.g., number of cells in the device, the density of an afibrotic compound disposed on the surface and/or in a barrier compartment of the device, the density of a CBS in the cell-containing compartment.

In an embodiment, the desired biological response upon implant of the implantable element into a subject is a lower amount of pericapsular fibrotic overgrowth (PFO) compared with the amount of PFO observed for a control implantable element (e.g., defined as an otherwise identical implantable element except that the cell does not have the reduction in the MHC class I protein complex). An effective amount may comprise the amount of therapeutic agent secreted by the engineered mammalian cells described herein. An effective amount encompasses therapeutic and prophylactic treatment.

“Effective amount”, as used herein, refers to an amount of genetically modified cells (e.g., derived from human cells (e.g., epithelial cells)) producing an exogenous polypeptide or a device preparation producing the polypeptide that is sufficient to elicit a desired biological response. In an embodiment, the desired biological response is an increase in levels of the exogenous polypeptide within the cells, or for secreted polypeptides, in a tissue sample removed from a subject treated with (e.g., implanted with) the genetically modified cells, a device or a device preparation containing such cells. As will be appreciated by those of ordinary skill in this art, the effective amount may vary depending on such factors as the desired biological endpoint, the pharmacokinetics of the exogenous polypeptide, composition or device, the condition being treated, the mode of administration, and the age and health of the subject. An effective amount encompasses therapeutic and prophylactic treatment.

An “endogenous nucleic acid” as used herein, is a nucleic acid that occurs naturally in a subject cell.

An “endogenous polypeptide,” as used herein, is a polypeptide that occurs naturally in a subject cell.

“Engineered human cell” and “genetically modified human cell”, may be used interchangeably herein, and each term means a human cell (e.g., an epithelial cell) having a non-naturally occurring genetic alteration (e.g., in the cellular genome), and typically comprises an exogenous nucleic acid sequence (e.g., DNA or RNA) not present (or present at a different level than) in an otherwise similar human cell (e.g., epithelial cell) that is not engineered. In an embodiment, an engineered human cell (e.g., engineered RPE cell) comprises an exogenous nucleic acid encoding a polypeptide, e.g., a therapeutic protein. In an embodiment, the exogenous nucleic acid sequence is chromosomal (e.g., the exogenous nucleic acid sequence is an exogenous sequence disposed in endogenous chromosomal sequence) or is extra chromosomal (e.g., a non-integrated expression vector). In an embodiment, the exogenous nucleic acid sequence comprises an RNA sequence, e.g., an mRNA. In an embodiment, the exogenous nucleic acid sequence comprises a chromosomal or extra-chromosomal exogenous nucleic acid sequence that comprises a sequence which is expressed as RNA, e.g., mRNA or a regulatory RNA. In an embodiment, the exogenous nucleic acid sequence comprises a first chromosomal or extra-chromosomal exogenous nucleic acid sequence that modulates the conformation or expression of a second nucleic acid sequence the second nucleic acid sequence can be exogenous or endogenous. For example, an engineered cell can comprise an exogenous nucleic acid that controls the expression of an endogenous sequence. In an embodiment, the engineered cell comprises an exogenous nucleic acid sequence which comprises a codon optimized coding sequence for a polypeptide of interest and achieves higher expression of the polypeptide than a naturally-occurring coding sequence. The codon optimized coding sequence may be generated using a commercially available algorithm, e.g., GeneOptimizer (ThermoFisher Scientific), OptimumGene™ (GenScript, Piscataway, NJ USA), GeneGPS® (ATUM, Newark, CA USA), or Java Codon Adaptation Tool (JCat, www.jcat.de, Grote, A. et al., Nucleic Acids Research, Vol 33, Issue suppl 2, pp. W526-W531 (2005). In an embodiment, an engineered cell (e.g., engineered epithelial cell, e.g., engineered RPE cell, e.g., engineered ARPE-19 cell) is cultured from a monoclonal cell line. In some embodiments, the engineered cell is not an islet cell, as defined herein.

An “exogenous nucleic acid,” as used herein, is a nucleic acid that does not occur naturally in a subject cell.

An “exogenous polypeptide,” as used herein, is a polypeptide that is encoded by an exogenous nucleic acid in a subject cell. Reference to an amino acid position of a specific sequence means the position of said amino acid in a reference amino acid sequence, e.g., sequence of a full-length mature (after signal peptide cleavage) wild-type protein (unless otherwise stated), and does not exclude the presence of variations, e.g., deletions, insertions and/or substitutions at other positions in the reference amino acid sequence.

“Factor VII protein” or “FVII protein” as used herein, means a polypeptide that comprises the amino acid sequence of a naturally occurring factor VII protein or variant thereof that has a FVII biological activity, e.g., promoting blood clotting, as determined by an art-recognized assay, unless otherwise specified. Naturally occurring FVII exists as a single chain zymogen, a zymogen-like two-chain polypeptide and a fully activated two-chain form (FVIIa).

In some embodiments, reference to FVII includes single-chain and two-chain forms thereof, including zymogen-like and FVIIa. FVII proteins that may be produced by a genetically modified cell described herein (e.g., derived from a human epithelial cell line, e.g., the ARPE-19 cell line), include wild-type primate (e.g., human), porcine, canine, and murine proteins, as well as variants of such wild-type proteins, including fragments, mutants, variants with one or more amino acid substitutions and/or deletions. In some embodiments, a variant FVII protein is capable of being activated to the fully activated two-chain form (Factor VIIa) that has at least 50%, 75%, 90% or more (including >100%) of the activity of wild-type Factor VIIa. Variants of FVII and FVIIa are known, e.g., marzeptacog alfa (activated) (MarzAA) and the variants described in European Patent No. 1373493, U.S. Pat. Nos. 7,771,996, 9,476,037 and US published application No. US20080058255.

Factor VII biological activity may be quantified by an art recognized assay, unless otherwise specified. For example, FVII biological activity in a sample of a biological fluid, e.g., plasma, may be quantified by (i) measuring the amount of Factor Xa produced in a system comprising tissue factor (TF) embedded in a lipid membrane and Factor X (Persson et al., J. Biol. Chem. 272:19919-19924, 1997); (ii) measuring Factor X hydrolysis in an aqueous system; (iii) measuring its physical binding to TF using an instrument based on surface plasmon resonance (Persson, FEBS Letts. 413:359-363, 1997); or (iv) measuring hydrolysis of a synthetic substrate; and/or (v) measuring generation of thrombin in a TF-independent in vitro system. In an embodiment, FVII activity is assessed by a commercially available chromogenic assay (BIOPHEN FVII, HYPHEN BioMed Neuville sur Oise, France), in which the biological sample containing FVII is mixed with thromboplastin calcium, Factor X and SXa-11 (a chromogenic substrate specific for Factor Xa.

“Factor VIII protein” or “FVIII protein” as used herein, means a polypeptide that comprises the amino acid sequence of a naturally occurring factor VIII polypeptide or variant thereof that has an FVIII biological activity, e.g., coagulation activity, as determined by an art-recognized assay, unless otherwise specified. FVIII proteins that may be expressed by a genetically modified cell described herein (e.g., derived from a human epithelial cell line, e.g., the ARPE-19 cell line), include wild-type primate (e.g., human), porcine, canine, and murine proteins, as well as variants of such wild-type proteins, including fragments, mutants, variants with one or more amino acid substitutions and/or deletions, B-domain deletion (BDD) variants, single chain variants and fusions of any of the foregoing wild-type or variants with a half-life extending polypeptide. In an embodiment, the cells are engineered to encode a precursor factor VIII polypeptide (e.g., with the signal sequence) with a full or partial deletion of the B domain. In an embodiment, the cells are engineered to encode a single chain factor VIII polypeptide which contains a variant FVIII protein preferably has at least 50%, 75%, 90% or more (including >100%) of the coagulation activity of the corresponding wild-type factor VIII. Assays for measuring the coagulation activity of FVIII proteins include the one stage or two stage coagulation assay (Rizza et al., 1982, Coagulation assay of FVIII:C and FIXa in Bloom ed. The Hemophelias. NY Churchill Livingston 1992) or the chromogenic substrate FVIII:C assay (Rosen, S. 1984. Scand J Haematol 33:139-145, suppl.).

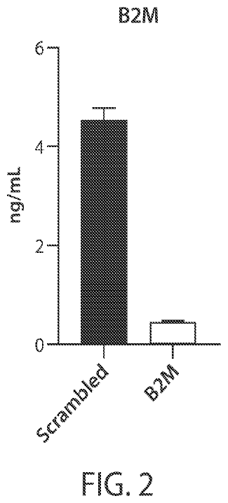

A number of FVIII-BDD variants are known, and include, e.g., variants with the full or partial B-domain deletions disclosed in any of the following U.S. Pat. No. 4,868,112 (e.g., col. 2, line 2 to col. 19, line 21 and table 2); U.S. Pat. No. 5,112,950 (e.g., col. 2, lines 55-68, FIG. 2, and example 1), U.S. Pat. No. 5,171,844 (e.g., col. 4, line 22 to col. 5, line 36); U.S. Pat. No. 5,543,502 (e.g., col. 2, lines 17-46); U.S. Pat. Nos. 5,595,886; 5,610,278; 5,789,203 (e.g., col. 2, lines 26-51 and examples 5-8); U.S. Pat. No. 5,972,885 (e.g., col. 1, lines 25 to col. 2, line 40); U.S. Pat. No. 6,048,720 (e.g., col. 6, lines 1-22 and example 1); U.S. Pat. Nos. 6,060,447; 6,228,620; 6,316,226 (e.g., col. 4, line 4 to col. 5, line 28 and examples 1-5); U.S. Pat. Nos. 6,346,513; 6,458,563 (e.g., col. 4, lines 25-53) and U.S. Pat. No. 7,041,635 (e.g., col. 2, line 1 to col. 3, line 19, col. 3, line 40 to col. 4, line 67, col. 7, line 43 to col. 8, line 26, and col. 11, line 5 to col. 13, line 39).

In some embodiments, a FVIII-BDD protein produced by a genetically modified cell described herein (e.g., derived from a human epithelial cell line, e.g., the ARPE-19 cell line) has one or more of the following deletions of amino acids in the B-domain: (i) most of the B domain except for amino-terminal B-domain sequences essential for intracellular processing of the primary translation product into two polypeptide chains (WO 91/09122); (ii) a deletion of amino acids 747-1638 (Hoeben R. C., et al. J Biol. Chem. 265 (13): 7318-7323 (1990)); amino acids 771-1666 or amino acids 868-1562 (Meulien P., et al. Protein Eng. 2(4):301-6 (1988); amino acids 982-1562 or 760-1639 (Toole et al., Proc. Natl. Acad. Sci. U.S.A. 83:5939-5942 (1986)); amino acids 797-1562 (Eaton et al., Biochemistry 25:8343-8347 (1986)); 741-1646 (Kaufman, WO 87/04187)), 747-1560 (Sarver et al., DNA 6:553-564 (1987)); amino acids 741-1648 (Pasek, WO 88/00831)), amino acids 816-1598 or 741-1689 (Lagner (Behring Inst. Mitt. (1988) No 82:16-25, EP 295597); a deletion that includes one or more residues in a furin protease recognition sequence, including any of the specific deletions recited in U.S. Pat. No. 9,956,269 at col. 10, line 65 to col. 11, line 36.

In other embodiments, a FVIII-BDD protein retains any of the following B-domain amino acids or amino acid sequences: (i) one or more N-linked glycosylation sites in the B-domain, e.g., residues 757, 784, 828, 900, 963, or optionally 943, first 226 amino acids or first 163 amino acids (Miao, H. Z., et al., Blood 103(a): 3412-3419 (2004), Kasuda, A., et al., J. Thromb. Haemost. 6: 1352-1359 (2008), and Pipe, S. W., et al., J.Thromb. Haemost. 9: 2235-2242 (2011).

In some embodiments, the FVIII-BDD protein is a single-chain variant generated by substitution or deletion of one or more amino acids in the furin protease recognition sequence LKRHQR that prevents proteolytic cleavage at this site, including any of the substitutions at the R1645 and/or R1648 positions described in U.S. Pat. Nos. 10,023,628, 9,394,353 and 9,670,267.

In some embodiments, any of the above FVIII-BDD proteins may further comprise one or more of the following variations: a F309S substitution to improve expression of the FVIII-BDD protein (Miao, H. Z., et al., Blood 103(a): 3412-3419 (2004); albumin fusions (WO 2011/020866); and Fc fusions (WO 04/101740).

All FVIII-BDD amino acid positions referenced herein refer to the positions in full-length human FVIII, unless otherwise specified.

“Factor IX protein” or “FIX protein”, as used herein, means a polypeptide that comprises the amino acid sequence of a naturally occurring factor IX protein or variant thereof that has a FIX biological activity, e.g., coagulation activity, as determined by an art-recognized assay, unless otherwise specified. FIX is produced as an inactive zymogen, which is converted to an active form by factor XIa excision of the activation peptide to produce a heavy chain and a light chain held together by one or more disulfide bonds. FIX proteins that may be produced by a genetically modified described herein (e.g., derived from an RPE cell line, e.g., the ARPE-19 cell line), include wild-type primate (e.g., human), porcine, canine, and murine proteins, as well as variants of such wild-type proteins, including fragments, mutants, variants with one or more amino acid substitutions and/or deletions and fusions of any of the foregoing wild-type or variant proteins with a half-life extending polypeptide. In an embodiment, cells are engineered to encode a full-length wild-type human factor IX polypeptide (e.g., with the signal sequence) or a functional variant thereof. A variant FIX protein preferably has at least 50%, 75%, 90% or more (including >100%) of the coagulation activity of wild-type factor VIX. Assays for measuring the coagulation activity of FIX proteins include the Biophen Factor IX assay (Hyphen BioMed) and the one stage clotting assay (activated partial thromboplastin time (aPTT), e.g., as described in EP 2 032 607, thrombin generation time assay (TGA) and rotational thromboelastometry, e.g., as described in WO 2012/006624.

A number of functional FIX variants are known and may be expressed by engineered cells encapsulated in a device described herein, including any of the functional FIX variants described in the following international patent publications: WO 02/040544 at page 4, lines 9-30 and page 15, lines 6-31; WO 03/020764 in Tables 2 and 3 at pages 14-24, and at page 12, lines 1-27; WO 2007/149406 at page 4, line 1 to page 19, line 11; WO 2007/149406 A2 at page 19, line 12 to page 20, line 9; WO 08/118507 at page 5, line 14 to page 6, line 5; WO 09/051717 at page 9, line 11 to page 20, line 2; WO 09/137254 at page 2, paragraph [006] to page 5, paragraph [011] and page 16, paragraph [044] to page 24, paragraph [057]; WO 09/130198 A2 at page 4, line 26 to page 12, line 6; WO 09/140015 at page 11, paragraph [0043] to page 13, paragraph [0053]; WO 2012/006624; WO 2015/086406.

In certain embodiments, the FIX polypeptide comprises a wild-type or variant sequence fused to a heterologous polypeptide or non-polypeptide moiety extending the half-life of the FIX protein. Exemplary half-life extending moieties include Fc, albumin, a PAS sequence, transferrin, CTP (28 amino acid C-terminal peptide (CTP) of human chorionic gonadotropin (hCG) with its 4 O-glycans), polyethylene glycol (PEG), hydroxyethyl starch (HES), albumin binding polypeptide, albumin-binding small molecules, or any combination thereof. An exemplary FIX polypeptide is the rFIXFc protein described in WO 2012/006624, which is an FIXFc single chain (FIXFc-sc) and an Fc single chain (Fc-sc) bound together through two disulfide bonds in the hinge region of Fc.

FIX variants also include gain and loss of function variants. An example of a gain of function variant is the “Padua” variant of human FIX, which has a L (leucine) at position 338 of the mature protein instead of an R (arginine) and has greater catalytic and coagulant activity compared to wild-type human FIX (Chang et al., J. Biol. Chem., 273:12089-94 (1998)). An example of a loss of function variant is an alanine substituted for lysine in the fifth amino acid position from the beginning of the mature protein, which results in a protein with reduced binding to collagen IV (e.g., loss of function).

“Islet cell”, as used herein, means a cell that comprises any naturally occurring or any synthetically created, or modified, cell that is intended to recapitulate, mimic or otherwise express, in part or in whole, the functions, in part or in whole, of the cells of the pancreatic islets of Langerhans. The term “islet cell” includes a glucose-responsive, insulin producing cell derived from a stem cell, e.g., from an induced pluripotent stem cell line.

“Polymer composition”, as used herein, is a composition (e.g., a solution, mixture) comprising one or more polymers. As a class, “polymers' includes homopolymers, heteropolymers, co-polymers, block polymers, block co-polymers and can be both natural and synthetic. Homopolymers contain one type of building block, or monomer, whereas co-polymers contain more than one type of monomer.

“Polypeptide”, as used herein, refers to a polymer comprising amino acid residues linked through peptide bonds and having at least two, and in some embodiments, at least 3, 4, 5, 10, 50, 75,100, 150 or 200 amino acid residues.

“Prevention,” “prevent,” and “preventing” as used herein refers to a treatment that comprises administering a composition (or preparation) of devices encapsulating genetically modified cells that express an exogenous polypeptide, prior to the onset of one or more symptoms of a disease or condition that is amenable to treatment with the exogenous polypeptide, to preclude the physical manifestation of the symptom(s). In some embodiments, “prevention,” “prevent,” and “preventing” require that signs or symptoms of a disease or condition have not yet developed or have not yet been observed.

“RPE cell” as used herein refers to a cell having one or more of the following characteristics: a) it comprises a retinal pigment epithelial cell (RPE) (e.g., cultured using an RPE cell line, e.g., the ARPE-19 cell line (ATCC© CRL-2302™)) or a cell derived or engineered therefrom, e.g., by stably transfecting cells cultured from the ARPE-19 cell line with an exogenous sequence that encodes a polypeptide of interest or inserting the exogenous sequence into one of the specific OCR insertion sites described herein, a cell derived from a primary cell culture of RPE cells, a cell isolated directly (without long term culturing, e.g., less than 5 or 10 passages or rounds of cell division since isolation) from naturally occurring RPE cells, e.g., from a human or other mammal, a cell derived from a transformed, an immortalized, or a long term (e.g., more than 5 or 10 passages or rounds of cell division) RPE cell culture; b) a cell that has been obtained from a less differentiated cell, e.g., a cell developed, programmed, or reprogramed (e.g., in vitro) into an RPE cell or a cell that is, except for any genetic engineering, substantially similar to one or more of a naturally occurring RPE cell or a cell from a primary or long term culture of RPE cells (e.g., the cell can be derived from an IPS cell); or c) a cell that has one or more of the following properties: i) it expresses one or more of the biomarkers CRALBP, RPE-65, RLBP, BEST1, or αB-crystallin; ii) it does not express one or more of the biomarkers CRALBP, RPE-65, RLBP, BEST1, or αB-crystallin; iii) it is naturally found in the retina and forms a monolayer above the choroidal blood vessels in the Bruch's membrane; or iv) it is responsible for epithelial transport, light absorption, secretion, and immune modulation in the retina; or v) it has been created synthetically, or modified from a naturally occurring cell, to have the same or substantially the same genetic content, and optionally the same or substantially the same epigenetic content, as an immortalized RPE cell line (e.g., the ARPE-19 cell line (ATCC® CRL-2302™)). Other exemplary strains of RPE cells include ARPE-19-SEAP-2-neo cells, RPE-J cells, and hTERT RPE-1 cells. In an embodiment, an RPE described herein is engineered, e.g., to have a new property, e.g., the cell is genetically modified by inserting at least one exogenous transcription unit into one or more of the OCR locations described herein.

“Sequence identity” or “percent identical”, when used herein to refer to two nucleotide sequences or two amino acid sequences, means the two sequences are the same within a specified region, or have the same nucleotides or amino acids at a specified percentage of nucleotide or amino acid positions within the specified when the two sequences are compared and aligned for maximum correspondence over a comparison window or designated region. Sequence identity may be determined using standard techniques known in the art including, but not limited to, any of the algorithms described in US Patent Application Publication No. 2017/02334455 A1. In an embodiment, the specified percentage of identical nucleotide or amino acid positions is at least about 80%, 85%, 90%, 91%, 92%, 93%, 94%, 95%, 96%, 97%, 98%, 99% or higher.

“Spherical” as used herein means a device (e.g., a hydrogel capsule or other particle) having a curved surface that forms a sphere (e.g., a completely round ball) or sphere-like shape, which may have waves and undulations, e.g., on the surface. Spheres and sphere-like objects can be mathematically defined by rotation of circles, ellipses, or a combination around each of the three perpendicular axes, a, b, and c. For a sphere, the three axes are the same length. Generally, a sphere-like shape is an ellipsoid (for its averaged surface) with semi-principal axes within 10%, or 5%, or 2.5% of each other. The diameter of a sphere or sphere-like shape is the average diameter, such as the average of the semi-principal axes.

“Spheroid”, as that term is used herein to refer to a device (e.g., a hydrogel capsule or other particle), means the device has (i) a perfect or classical oblate spheroid or prolate spheroid shape or (ii) has a surface that roughly forms a spheroid, e.g., may have waves and undulations and/or may be an ellipsoid (for its averaged surface) with semi-principal axes within 100% of each other.

“Subject” as used herein refers to a human or non-human animal. In an embodiment, the subject is a human (i.e., a male or female) of any age group, e.g., a pediatric human subject (e.g., infant, child, adolescent) or adult human subject (e.g., young adult, middle-aged adult, or senior adult)). In an embodiment, the subject is a non-human animal, for example, a mammal (e.g., a mouse, a dog, a primate (e.g., a cynomolgus monkey or a rhesus monkey). In an embodiment, the subject is a commercially relevant mammal (e.g., cattle, pig, horse, sheep, goat, cat, or dog) or a bird (e.g., a commercially relevant bird such as a chicken, duck, goose, or turkey). In certain embodiments, the animal is a mammal. The animal may be a male or female and at any stage of development. A non-human animal may be a transgenic animal.

“Treatment,” “treat,” and “treating” as used herein refers to one or more of reducing, reversing, alleviating, delaying the onset of, or inhibiting the progress of one or more of a symptom, manifestation, or underlying cause, of a disease (e.g., hemophilia A). In an embodiment, treating comprises reducing, reversing, alleviating, delaying the onset of, or inhibiting the progress of a symptom or condition associated with the disease. In an embodiment, treating comprises increasing levels of a therapeutic polypeptide in at least one tissue of a subject in need thereof, e.g., in one or more of plasma, liver, kidney and heart. In some embodiments, “treatment,” “treat,” and “treating” require that signs or symptoms associated with the disease or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., due to a history of symptoms and/or genetic or other susceptibility factors). Treatment may also be continued after symptoms have resolved, for example, to delay or prevent recurrence. In some embodiments, treatment comprises prevention and in other embodiments it does not “Wild-type” (wt) refers to the natural form, including sequence, of a polynucleotide, polypeptide or protein in a species. A wild-type form is distinguished from a mutant form of a polynucleotide, polypeptide or protein arising from genetic mutation(s).

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₆, C₃-C₄, C₄-C₆, C₄-C₅ and C₅-C₆ alkyl.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”), 1 to 10 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents, e.g., for instance from 1 to 5 substituents, 1 to 3 substituents, or 1 substituent.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”), 2 to 8 carbon atoms (“C₂-C₈ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”), 2 to 8 carbon atoms (“C₂-C₈ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂-C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR_{C}R_{D}, or the like, it will be understood that the terms heteroalkyl and —CH₂O or —NR_{C}R_{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. An alkylene, alkenylene, alkynylene, or heteroalkylene group may be described as, e.g., a C₁-C₆-membered alkylene, C₂-C₆-membered alkenylene, C₂-C₆-membered alkynylene, or C₁-C₆-membered heteroalkylene, wherein the term “membered” refers to the non-hydrogen atoms within the moiety. In the case of heteroalkylene groups, heteroatoms can also occupy either or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, the terms “arylene” and “heteroarylene,” alone or as part of another substituent, mean a divalent radical derived from an aryl and heteroaryl, respectively.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro [4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, piperazinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl or thiomorpholinyl-1,1-dioxide. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Amino” as used herein refers to the radical —NR⁷⁰R⁷¹, wherein R⁷⁰ and R⁷¹ are each independently hydrogen, C₁-C₈ alkyl, C₃-C₁₀ cycloalkyl, C₄-C₁₀ heterocyclyl, C₆-C₁₀ aryl, and C₅-C₁₀ heteroaryl. In some embodiments, amino refers to NH₂.

As used herein, “cyano” refers to the radical —CN.

As used herein, “halo” or “halogen,” independently or as part of another substituent, mean, unless otherwise stated, a fluorine (F), chlorine (C₁), bromine (Br), or iodine (I) atom.

As used herein, “hydroxy” refers to the radical —OH.

Alkyl, alkenyl, alkynyl, heteroalkyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds of Formula (I) described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

Compounds of Formula (I) described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; P may be in any isotopic form, including ¹⁶O and ¹⁸O; and the like.

The term “pharmaceutically acceptable salt” is meant to include salts of the active compounds that are prepared with relatively nontoxic acids or bases, depending on the particular substituents found on the compounds described herein. When compounds of Formula (I) used to prepare devices of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al, Journal of Pharmaceutical Science 66: 1-19 (1977)). Certain specific compounds used in the devices of the present disclosure (e.g., a particle, a hydrogel capsule) contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

Devices of the present disclosure may contain a compound of Formula (I) in a prodrug form. Prodrugs are those compounds that readily undergo chemical changes under physiological conditions to provide the compounds useful for preparing devices in the present disclosure. Additionally, prodrugs can be converted to useful compounds of Formula (I) by chemical or biochemical methods in an ex vivo environment.

Certain compounds of Formula (I) described herein can exist in unsolvated forms as well as solvated forms, including hydrated forms. In general, the solvated forms are equivalent to unsolvated forms and are encompassed within the scope of the present disclosure. Certain compounds of Formula (I) described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0.

The term “tautomer” as used herein refers to compounds that are interchangeable forms of a compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

The symbol “ [symbol] ” as used herein refers to a connection to an entity, e.g., a polymer (e.g., hydrogel-forming polymer such as alginate) or surface of an implantable device, e.g., a particle, a hydrogel capsule. The connection represented by “ [symbol] ” may refer to direct attachment to the entity, e.g., a polymer or an implantable element, may refer to linkage to the entity through an attachment group. An “attachment group,” as described herein, refers to a moiety for linkage of a compound of Formula (I) to an entity (e.g., a polymer or an implantable element (e.g., a device) as described herein), and may comprise any attachment chemistry known in the art. A listing of exemplary attachment groups is outlined in Bioconjugate Techniques (3^{rd} ed, Greg T. Hermanson, Waltham, MA: Elsevier, Inc, 2013), which is incorporated herein by reference in its entirety. In some embodiments, an attachment group comprises alkyl, alkenyl, alkynyl, heteroalkyl, cycloalkyl, heterocyclyl, aryl, heteroaryl, —C(O)—, —OC(O)—, —N(R^{C})—, —N(R^{C})C(O)—, —C(O)N(R^{C})—, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, wherein each of R^{A}, R^{C}, R^{D}, R^{F}, R^{G}, x and y is independently as described herein. In some embodiments, an attachment group comprises an amine, ketone, ester, amide, alkyl. In some embodiments, an attachment group is a cross-linker. In some embodiments, the [symbol] attachment group is —C(O)(C₁-C₆₁, and R¹ is as described herein [symbol] . In some embodiments, the attachment group is —C(O)(C₁-C₆-alkylene)-, wherein alkylene is substituted with 1-2 alkyl groups (e.g., 1-2 methyl groups). In some embodiments, the attachment group is —C(O)C(CH₃)₂—. In some embodiments, the attachment group is —C(O)(methylene)-, wherein alkylene is substituted with 1-2 alkyl groups (e.g., 1-2 methyl groups). In some embodiments, the attachment group is —C(O)CH(CH₃)—. In some embodiments, the attachment group is —C(O)C(CH₃)—.

### Engineered Mammalian Cells

The present disclosure provides an engineered mammalian cell capable of modulating the level or function of the MHC class I protein complex, and optionally, the MHC class II protein complex and/or CITA. In an embodiment, the engineered mammalian cell reduces a level or function of the MHC class I protein complex, and optionally, the MHC class II protein complex and/or CIITA.

The MHC class I protein complex is a class of molecules present on the surface of nucleated cells that inform the host's immune system of the status of a particular antigen as being self or non-self. The MHC class I molecules display peptide fragments of cytotoxic proteins on the cell surface, which trigger an immune response within the host if the cytotoxic protein is derived from a non-self-source. In general, the MHC class I molecules are heterodimeric proteins that consist of two polypeptide chains. The alpha chain is polymorphic, and is encoded by a human leukocyte antigen (1-1A) comprising one of HLA-A, HLA-B, or HLA-C. The beta chain comprises the beta-2-microglobulin (beta-2M) domain. The alpha and beta chain of each MHC class I molecule are noncovalently linked through the interaction of the beta-2M and one of the three plasma membrane-spanning domains of the alpha chain (alpha-3). The alpha chain also comprises two other domains: alpha-1 and alpha-2. In an embodiment, the engineered mammalian cell of the present disclosure comprises a reduced level or function in an MH1C class I protein complex or a component thereof, e.g., HLA-A, HLA-B, HLA-C, or beta-2M.

The groove between the alpha-1 and alpha-2 domains is the peptide-binding groove, in which peptides derived from cytosolic proteins are displayed. The groove comprises eight β-pleated sheets on the bottom and two α helices making up sides. The groove is flanked by tyrosine residues and creates closed ends that limit the size of peptides that can be bound within the groove. The peptide in the groove remains substantially bound there for the life of the class I molecule and is typically 8-9 amino acids in length. Self or foreign cytosolic proteins are degraded via the proteasome and transported into the lumen of the ER. In the ER, the peptides are loaded onto an MHC class 1 via the aid of a chaperone protein named tapasin. The peptide bound MHC class I is then transported to the cell's plasma membrane, where it presents the peptide to CD8+ T cell receptors (Becar M et al. (2022) Physiology. MHC Class 1. In: StatPearls [Internet], Treasure Island (FL): StatPearls Publishing; 2023 January-.)

In addition to its interaction with beta-2M, the plasma membrane-spanning alpha-3 domain interacts with the T cell receptor (TCR) co-receptor CD8, facilitating antigen-specific activation. Although binding of MHC class I to CD8 is about 100-fold weaker than binding of TCR to MHC class I, alpha-3-CD8 binding enhances the affinity ofTCR binding (Wooldridge et aL (2010)MHC Class I Molecules with Superenhanced CD8 Binding Properties Bypass the Requirement for Cognate TCR Recognition and Nonspecifically Activate CTLs, J. Immunol. 184:3357-3366).

Beta-2M is a non-glycosylated 12 kDa protein; one of its functions is to stabilize the MCI-C class I α-chain. Unlike the α-chain, the beta-2M does not span the membrane. The human beta-2M locus is on chromosome 15. The beta-2NM gene consists of4 exons and 3 introns. Circulating forms of beta-2M are present in the serum, urine, and other body fluids; thus, the non-covalently iIHC I-associated beta-2M can be exchanged with circulating beta-2M under physiological conditions. Beta-2M associates not only with the alpha chain of MHC class I molecules, but also with class I-like molecules such as CD1 (5 genes in humans), MR1, the neonatal Fc receptor (FcRn), and Qa-1 (a form of alloantigen).

In some embodiments, the engineered mammalian cell (e.g., ARPE-19) comprises a reduction in a level or function of an MHC class I protein complex or a component thereof, e.g., HLA-A, HLA-B, HLA-C, or beta-2M. In some embodiments, the engineered mammalian cell (e.g., ARPE-19) comprises a reduction in the alpha and/or beta chain of the MHC class I protein complex or component thereof. For example, in some embodiments, the engineered mammalian cell (e.g., ARPE-19) comprises a reduction in a level or function of the alpha-1 domain. In some embodiments, the level or function of the alpha-1 domain is reduced by about 10% (e.g., about 20%, 30%, 40%, 50%, 60%, 70%, 80%, 90%, or 100%). For example, in some embodiments, the level or function of the alpha-1 domain is reduced by about 20%. In some embodiments, the level or function of the alpha-1 domain is reduced by about 30%. In some embodiments, the level or function of the alpha-1 domain is reduced by about 40%. In some embodiments, the level or function of the alpha-1 domain is reduced by about 50%. In some embodiments, the level or function of the alpha-1 domain is reduced by about 60%. In sone embodiments, the level or function of the alpha-1 domain is reduced by about 70%. In some embodiments, the level or function of the alpha-1 domain is reduced by about 80%. In some embodiments, the level or function of the alpha-1 domain is reduced by about 90%. In some embodiments, the level or function of the alpha-1 domain is reduced by about 100%.

In some embodiments, the level or function of the alpha-1 domain is reduced by at least 5% (e.g., at least 10%, 20%, 30%, 40%, 50%, 60%, 70%, 80%, 90%, 95%, 99%, 99.9% or more). For example, in some embodiments, the level or function of the alpha-1 domain is reduced by at least 10%. In some embodiments, the level or function of the alpha-1 domain is reduced by at least 20%. In some embodiments, the level or function of the alpha-1 domain is reduced by at least 30%. In some embodiments, the level or function of the alpha-domain is reduced by at least 40% in some embodiments, the level or function of the alpha-1 domain is reduced by at least 50%. In some embodiments, the level or function of the alpha-1 domain is reduced by at least 60%. In some embodiments, the level or function of the alpha-1 domain is reduced by at least 70% In some embodiments, the level or function of the alpha-1 domain is reduced by at least 80%. In some embodiments, the level or function of the alpha-1 domain is reduced by at least 90%. In some embodiments, the level or function of the alpha-1 domain is reduced by at least 95%. In some embodiments, the level or function of the alpha-1 domain is reduced by at least 99%, In some embodiments, the level or function of the alpha-1 domain is reduced by at least 99.9%. In some embodiments, the level or function of the alpha-1 domain is reduced by more than 99.9%.

In some embodiments, the engineered mammalian cell (e.g., ARPE-19) comprises a reduction in a level or function of the alpha-2 domain. In some embodiments, the level or function of the alpha-2 domain is reduced by about 10% (e.g., about 20%, 30%, 40%, 50%, 60%, 70%, 80%, 90%, or 100%). For example, in some embodiments, the level or function of the alpha-2 domain is reduced by about 20%. In some embodiments, the level or function of the alpha-2 domain is reduced by about 30%. In some embodiments, the level or function of the alpha-2 domain is reduced by about 40%, In some embodiments, the level or function of the alpha-2 domain is reduced by about 50%. In some embodiments, the level or function of the alpha-2 domain is reduced by about 60%. In some embodiments, the level or function of the alpha-2 domain is reduced by about 70%. In some embodiments, the level or function of the alpha-2 domain is reduced by about 80%. In some embodiments, the level or function of the alpha-2 domain is reduced by about 90%. In some embodiments, the level or function of the alpha-2 domain is reduced by about 100%.

In some embodiments, the level or function of the alpha-2 domain is reduced by at least 5% (e.g., at least 10%, 20%, 30%, 40%, 50%, 60%, 70%, 80%, 90%, 95%, 99%, 99.9% or more). For example, in some embodiments, the level or function of the alpha-2 domain is reduced by at least 10%. In some embodiments, the level or function of the alpha-2 domain is reduced by at least 20%. In some embodiments, the level or function of the alpha-2 domain is reduced by at least 30%. In some embodiments, the level or function of the alpha-2 domain is reduced by at least 40%. In some embodiments, the level or function of the alpha-2 domain is reduced by at least 50%. In some embodiments, the level or function of the alpha-2 domain is reduced by at least 60%, In some embodiments, the level or function of the alpha-2 domain is reduced by at least 70%. In some embodiments, the level or function of the alpha-2 domain is reduced by at least 80%. In some embodiments, the level or function of the alpha-2 domain is reduced by at least 90%. In some embodiments, the level or function of the alpha-2 domain is reduced by at least 95%. In some embodiments, the level or function of the alpha-2 domain is reduced by at least 99%. In some embodiments, the level or function of the alpha-2 domain is reduced by at least 99.9%. In some embodiments, the level or function of the alpha-2 domain is reduced by more than 99.9%.

In some embodiments, the engineered mammalian cell (e.g., ARPE-19) comprises a reduction in a level or function of the alpha-3 domain. In some embodiments, the level or function of the alpha-3 domain is reduced by about 10% (e.g., about 20%, 30%, 40%, 50%, 60%, 70%, 80%, 90%, or 100%). For example, in some embodiments, the level or function of the alpha-3 domain is reduced by about 20% In some embodiments, the level or function of the alpha-3 domain is reduced by about 30%. In some embodiments, the level or function of the alpha-3 domain is reduced by about 40%. In some embodiments, the level or function of the alpha-3 domain is reduced by about 50%. In some embodiments, the level or function of the alpha-3 domain is reduced by about 60%. In some embodiments, the level or function of the alpha-3 domain is reduced by about 70%. In some embodiments, the level or function of the alpha-3 domain is reduced by about 80%. In some embodiments, the level or function of the alpha-3 domain is reduced by about 90%. In some embodiments, the level or function of the alpha-3 domain is reduced by about 100%.

In some embodiments, the level or function of the alpha-3 domain is reduced by at least 5% (e g., at least 10%, 20%, 30%, 40%, 50%, 60%, 70%, 80%, 90%, 95%, 99%, 99.9% or more). For example, in some embodiments, the level or function of the alpha-3 domain is reduced by at least 10%. In some embodiments, the level or function of the alpha-3 domain is reduced by at least 20%. In some embodiments, the level or function of the alpha-3 domain is reduced by at least 30%. In some embodiments, the level or function of the alpha-3 domain is reduced by at least 40%. In some embodiments, the level or function of the alpha-3 domain is reduced by at least 50%. In some embodiments, the level or function of the alpha-3 domain is reduced by at least 60%. In some embodiments, the level or function of the alpha-3 domain is reduced by at least 70%. In some embodiments, the level or function of the alpha-3 domain is reduced by at least 80%. In some embodiments, the level or function of the alpha-3 domain is reduced by at least 90%. In some embodiments, the level or function of the alpha-3 domain is reduced by at least 95%. In some embodiments, the level or function of the alpha-3 domain is reduced by at least 99%. In some embodiments, the level or function of the alpha-3 domain is reduced by at least 99.9%. In some embodiments, the level or function of the alpha-3 domain is reduced by more than 99.9%.

In some embodiments, the engineered mammalian cell (e.g., ARPE-19) comprises a reduction in a level or function of the beta-2M domain. In some embodiments, the level or function of the beta-2M domain is reduced by about 10% (e.g., about 20%, 30%, 40%, 50%, 60%, 70%, 80%, 90%, or 100%). For example, in some embodiments, the level or function of the beta-2M domain is reduced by about 20%. In some embodiments, the level or function of the beta-2M domain is reduced by about 30%. In some embodiments, the level or function of the beta-2M domain is reduced by about 40%. In some embodiments, the level or function of the beta-2M domain is reduced by about 50%. In some embodiments, the level or function of the beta-2M domain is reduced by about 60%. In some embodiments, the level or function of the beta-2M domain is reduced by about 70%. In some embodiments, the level or function of the beta-2M domain is reduced by about 80%. In some embodiments, the level or function of the beta-2M domain is reduced by about 90%. In some embodiments, the level or function of the beta-2M domain is reduced by about 100%.

In some embodiments, the level or function of the beta-2M domain is reduced by at least 5% (e.g., at least 10%, 20%, 30%, 40%, 50%, 60%, 70%, 80%, 90%, 95%, 99%, 99.9% or more). For example, in some embodiments, the level or function of the beta-2M domain is reduced by at least 10%. In some embodiments, the level or function of the beta-2M domain is reduced by at least 20%. In some embodiments, the level or function of the beta-2M domain is reduced by at least 30%. In some embodiments, the level or function of the beta-2M domain is reduced by at least 40%. In some embodiments, the level or function of the beta-2M domain is reduced by at least 50%. In some embodiments, the level or function of the beta-2M domain is reduced by at least 60%. In some embodiments, the level or function of the beta-2M domain is reduced by at least 70%. In some embodiments, the level or function of the beta-2M domain is reduced by at least 80%. In some embodiments, the level or function of the beta-2M domain is reduced by at least 90%. In some embodiments, the level or function of the beta-2M domain is reduced by at least 95%. In some embodiments, the level or function of the beta-2M domain is reduced by at least 99%. In some embodiments, the level or function of the beta-2M domain is reduced by at least 99.9%. In some embodiments, the level or function of the beta-2M domain is reduced by more than 99.9%.

In some embodiments, the reducing the level or function of substantially decreases, prevents, or inhibits, e.g., by 10%, 20%, 30%, 40%, 50%, 75%, 90%, 95%, or more, the interaction (e g., binding) of beta-2M to a MHC class I protein complex or component thereof or an MHC class I-like molecule or component (e.g., CD1, MR1, FcRn, and Qa-1). For example, in some embodiments, the reducing the level or function of substantially decreases, prevents, or inhibits the interaction (e.g., binding) of beta-2M to an MHC class I protein complex or component thereof. In some embodiments, the reducing the level or function of substantially decreases, prevents, or inhibits the interaction (e.g., binding) of beta-2M to the alpha-1 domain of an MHC class I protein. In some embodiments, the reducing the level or function of substantially decreases, prevents, or inhibits the interaction (e.g., binding) of beta-2M to the alpha-2 domain of an MHC class I protein. In some embodiments, the reducing the level or function of substantially decreases, prevents, or inhibits the interaction (e.g., binding) of beta-2M to the alpha-3 domain of an MHC class I protein. In some embodiments, the reducing the level or function of substantially decreases, prevents, or inhibits the interaction (e.g., binding) of beta-2M to an MHC class I-like molecule or component (e.g., CD1, MR1, FcRn, and Qa-1). In some embodiments, the reducing the level or function of substantially decreases, prevents, or inhibits the interaction (e.g., binding) of beta-2M to CDL In some embodiments, the reducing the level or function of substantially decreases, prevents, or inhibits the interaction (e.g., binding) of beta-2M to MR1, In some embodiments, the reducing the level or function of substantially decreases, prevents, or inhibits the interaction (e.g., binding) of beta-2M to FcRn. In some embodiments, the reducing the level or function of substantially decreases, prevents, or inhibits the interaction (e.g., binding) of beta-2M to Qa-1.

In some embodiments, the reducing the level or function of substantially decreases, prevents, or inhibits, e.g., by 10%, 20%, 30%, 40%, 50%, 75%, 90%, 95%, or more, the interaction (e.g., binding) of the alpha-3 domain to the TCR co-receptor CD8.

HLA-A interacts with calnexin, calreticulin, transporter associated with antigen processing (TAP), tapasin, the thiol-disulfide oxidoreductase ERp57 enzyme, and any cytosolic peptide bound within its peptide-binding groove. In some embodiments, the reducing the level or function of substantially decreases, prevents, or inhibits, e.g., by 10%, 20%, 30%, 40%, 50%, 75%, 90%, 95%, or more, the interaction (e.g., binding) of F-LA-A to calnexin, calreticulin, TAP, tapasin, an ERp57 enzyme, and/or any cytosolic peptide bound within its peptide-binding groove. For example, in some embodiments, the reducing the level or function of substantially decreases, prevents, or inhibits the interaction (e.g., binding) of ILA-A to calnexin. In some embodiments, the reducing the level or function of substantially decreases, prevents, or inhibits the interaction (e.g., binding) of HLA-A to calreticulin. In some embodiments, the reducing the level or function of substantially decreases, prevents, or inhibits the interaction (e.g., binding) of HLA-A to TAP. In some embodiments, the reducing the level or function of substantially decreases, prevents, or inhibits the interaction (e.g., binding) of HLA-A to TAP-1. In some embodiments, the reducing the level or function of substantially decreases, prevents, or inhibits the interaction (e.g., binding) of HLA-A to TAP-2. In some embodiments, the reducing the level or function of substantially decreases, prevents, or inhibits the interaction (e.g., binding) of HLA-A to tapasin. In some embodiments, the reducing the level or function of substantially decreases, prevents, or inhibits the interaction (e.g., binding) of HLA-A to ERp57. In some embodiments, the reducing the level or function of substantially decreases, prevents, or inhibits the interaction (e.g., binding) of HLA-A to a cytosolic peptide bound within its peptide-binding groove.

HLA-C interacts with killer cell immunoglobulin-like receptor 2DL1 (KIR2DL1) and the leukocyte immunoglobulin-like receptor family (e.g., leukocyte immunoglobulin-like receptor subfamily A member 1 (LILRA1) and LILRA3). In some embodiments, the reducing the level or function of substantially decreases, prevents, or inhibits, e.g., by 10%, 20%, 30%, 40%, 50%, 75%, 90%, 95%, or more, the interaction (e.g., binding) of ILA-C to KIR2DL1 and the leukocyte immunoglobulin-like receptor family (e.g., LILRA1 and LILRA3). For example, in some embodiments, the reducing the level or function of substantially decreases, prevents, or inhibits the interaction (e.g., binding) of HLA-C to KIR2DL1. In some embodiments, the reducing the level or function of substantially decreases, prevents, or inhibits the interaction (e.g., binding) of HLA-C to the leukocyte immunoglobulin-like receptor family. In some embodiments, the reducing the level or function of substantially decreases, prevents, or inhibits the interaction (e.g., binding) of HLA-C to LILRA1, In some embodiments, the reducing the level or function of substantially decreases, prevents, or inhibits the interaction (e.g., binding) of HLA-C to LILRA3.

In some embodiments, the reducing the level or function of the MHC class I protein complex or a component thereof, e.g., HLA-A, HLA-B, HLA-C, or beta-2M, results in reduced antigen presentation, thereby reducing and/or abrogating the recruitment of immune cells, e.g., T cells and NK cells.

The HLA-A gene is located on the short arm of chromosome 6 and encodes the larger, alpha-chain, constituent of HLA-A. Variation of the HLA-A alpha-chain is key to HLA function. This variation promotes genetic diversity in the population Since each HLA has a different affinity for peptides of certain structures, a greater variety of HLAs means that a greater variety of antigens can be presented on the cell surface, enhancing the likelihood that a subset of the population will be resistant to a given foreign invader. This decreases the likelihood that a single pathogen has the capability to wipe out the entire human population.

Each individual can express up to two types of HLA-A, one from each of their parents. Some individuals will inherit the same HLA-A from both parents, decreasing their individual HLA diversity; however, the majority of individuals will receive two different copies of HLA-A. This same pattern follows for all HLA groups (Fix et al. (1998). HLA Matching, Antibodies, and You. Kidney Transplantation: Past, Present, and Future. Lniversity of Michigan Medical Center/Stanford University). In other words, every single person can only express either one or two of the 2432 known HLA-A alleles.

The HLA-B gene is located on the short (p) arm of chromosome 6 at cytoband 21.3 and encodes the larger, alpha-chain, constituent of HLA-B. Similar to HLA-A, variation of HLA-B alpha-chain is key to HLA function. HLA-C is a locus on chromosome 6, which encodes for many HLA-C alleles that are Class-I MHC receptors. HLA-C, localized proximal to the HLA-B locus, is located on the distal end of the HLA region. Most HLA-C:B haplotypes are in strong linkage disequilibrium and many are as ancient as the human species itself.

In some embodiments, the reducing the level or function of the MHC class I protein complex or a component thereof, e.g., HLA-A, HLA-B, HLA-C, or beta-2M, comprises mutating one or more nucleotides in the nucleotide sequence of one or more genes selected from HLA-A, HLA-B, HLA-C, or beta-21V. A nucleotide mutation may comprise a nucleotide deletion, addition, and/or substitution. Such a mutation, as described herein, may result in a reduction in expression of the gene, e.g., by reducing, altering, or abrogating the transcription and/or splicing of the nucleotide sequence. For example, in some embodiments, the reducing the level or function of the MHC class I protein complex or a component thereof comprises mutating one or more nucleotides in the nucleotide sequence of the beta-2M gene. In some embodiments, the reducing the level or function of the MHC-C class I protein complex or a component thereof comprises mutating one or more nucleotides in the nucleotide sequence of the HLA-A gene. In some embodiments, the reducing the level or function of the MHC class I protein complex or a component thereof comprises mutating one or more nucleotides in the nucleotide sequence of the HLA-B gene. In some embodiments, the reducing the level or function of the MH C class I protein complex or a component thereof comprises mutating one or more nucleotides in the nucleotide sequence of the HLA-C gene.

In some embodiments, the nucleotide sequence of the HLA-A, HLA-B, HLA-C, and beta-2M genes comprises a nucleotide sequence provided in Table 5. In some embodiments, the nucleotide sequence of the HLA-A, HLA-B, HLA-C, and beta-2M genes comprises a sequence having at least 60% (e.g., at least 65%, 70%, 75%, 80%, 85%, 90%, 95%, 99%, 99.9%, or greater) sequence identity to a nucleotide sequence provided in Table 5. For example, in some embodiments, the nucleotide sequence of the HLA-A, HLA-B, HLA-C, and beta-2M genes comprises a sequence having at least 65% sequence identity to a nucleotide sequence provided in Table 5. In some embodiments, the nucleotide sequence of the HLA-A, HLA-B, HLA-C, and beta-2M genes comprises a sequence having at least 70% sequence identity to a nucleotide sequence provided in Table 5. In some embodiments, the nucleotide sequence of the HLA-A, HLA-B, HLA-C, and beta-2M genes comprises a sequence having at least 75% sequence identity to a nucleotide sequence provided in Table 5. In some embodiments, the nucleotide sequence of the HLA-A, HLA-B, HLA-C, and beta-2M genes comprises a sequence having at least 80% sequence identity to a nucleotide sequence provided in Table 5. In some embodiments, the nucleotide sequence of the HLA-A, HLA-B, HLA-C, and beta-2M genes comprises a sequence having at least 85% sequence identity to a nucleotide sequence provided in Table 5. In some embodiments, the nucleotide sequence of the HLA-A, HLA-B, HLA-C, and beta-2M genes comprises a sequence having at least 90% sequence identity to a nucleotide sequence provided in Table 5. In some embodiments, the nucleotide sequence of the HLA-A, HLA-B, HLA-C, and beta-2M genes comprises a sequence having at least 95% sequence identity to a nucleotide sequence provided in Table 5 In some embodiments, the nucleotide sequence of the HLA-A, HLA-B, HLA-C, and beta-2M genes comprises a sequence having at least 99% sequence identity to a nucleotide sequence provided in Table 5. In some embodiments, the nucleotide sequence of the HLA-A, HLA-B, HLA-C, and beta-2M genes comprises a sequence having at least 99.9% sequence identity to a nucleotide sequence provided in Table 5. In some embodiments, the nucleotide sequence of the HLA-A, HLA-B, LA-C, and beta-2M genes comprises a sequence having greater than 99.9% sequence identity to a nucleotide sequence provided in Table 5.

In some embodiments, the nucleotide sequence of the HLA-A, HLA-B, HLA-C, and beta-2M genes comprises a sequence having at least 60% (e.g., at least 65%, 70%, 75%, 80%, 85%, 90%, 95%, 99%, 99.9%, or greater) sequence homology to a nucleotide sequence provided in Table 5. For example, in some embodiments, the nucleotide sequence of the HLA-A, HLA-B, HIL A-C, and beta-2M genes comprises a sequence having at least 65% sequence homology to a nucleotide sequence provided in Table 5. In some embodiments, the nucleotide sequence of the HLA-A, HLA-B, HLA-C, and beta-2M genes comprises a sequence having at least 70% sequence homology to a nucleotide sequence provided in Table 5. In some embodiments, the nucleotide sequence of the HLA-A, HLA-B, HLA-C, and beta-2M genes comprises a sequence having at least 75% sequence homology to a nucleotide sequence provided in Table 5. In some embodiments, the nucleotide sequence of the HLA-A, HLA-B, HLA-C, and beta-2M genes comprises a sequence having at least 80% sequence homology to a nucleotide sequence provided in Table 5. In some embodiments, the nucleotide sequence of the HLA-A, HLA-B, HLA-C, and beta-2M genes comprises a sequence having at least 85% sequence homology to a nucleotide sequence provided in Table 5. In some embodiments, the nucleotide sequence of the HLA-A, HLA-B, HLA-C, and beta-2M genes comprises a sequence having at least 90% sequence homology to a nucleotide sequence provided in Table 5. In some embodiments, the nucleotide sequence of the HLA-A, HA-B, HA-C, and beta-2M genes comprises a sequence having at least 95% sequence homology to a nucleotide sequence provided in Table 5. In some embodiments, the nucleotide sequence of the HLA-A, HLA-B, HLA-C, and beta-2M genes comprises a sequence having at least 99% sequence homology to a nucleotide sequence provided in Table 5. In some embodiments, the nucleotide sequence of the HLA-A, HLA-B, HLA-C, and beta-2M genes comprises a sequence having at least 99.9% sequence homology to a nucleotide sequence provided in Table 5. In some embodiments, the nucleotide sequence of the HLA-A, HLA-B, HLA-C, and beta-2M genes comprises a sequence having greater than 99.9% sequence homology to a nucleotide sequence provided in Table 5.

In an embodiment, the engineered mammalian cell described herein (e.g., ARPE-19), comprises a reduction in the expression of a MHC class I component by about 0.05%, 0.1%, 0.5%, 0.75%, 1%, 2%, 3%, 4%, 5%, 6%, 7%, 8%, 9%, 10%, 15%, 20%, 25%, 30%, 35%, 40%, 45%, 50%, 55%, 60%, 65% 70%, 75% 80%, 85%, 90% 95%, 99%, or more, e.g., as compared to an engineered mammalian cell that is substantially identical to or identical to the engineered mammalian cell except that it does not comprise a reduction in the level of an MHC class I component. In an embodiment, the engineered mammalian cell described herein (e.g., ARPE-19), comprises a reduction in the expression of a MHC class I component between 1-25%, 5-25%, 10-25%, 25-50%, 25-75%, 50-75%, or 75-100%, e.g., as compared to an engineered mammalian cell that is substantially identical to or identical to the engineered mammalian cell except that it does not comprise a reduction in the level of an MHC class I component. In an embodiment, the engineered mammalian cell described herein (e.g., ARPE-19), comprises a reduction in the expression of a MHC class I component greater than about 50%, 75%, or 90%, e.g., as compared to an engineered mammalian cell that is substantially identical to or identical to the engineered mammalian cell except that it does not comprise a reduction in the level of an MHC class I component.

In an embodiment, the engineered mammalian cell described herein (e.g., ARPE-19), comprises a reduction in the function of a MHC class I component by about 0.05%, 0.1%, 0.5%, 0.75%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the function of an MHC class I component. In an embodiment, the engineered mammalian cell described herein (e.g., ARPE-19), comprises a reduction in the function of a MHC class I component between 1-25%, 5-25%, 10-25%, 25-50%, 25-75%, 50-75%, or 75-100%, e.g., as compared to an engineered mammalian cell that is substantially identical to or identical to the engineered mammalian cell except that it does not comprise a reduction in the function of an MHC class I component. In an embodiment, the engineered mammalian cell described herein (e.g., ARPE-19), comprises a reduction in the function of a MHC class I component greater than about 50%, 75%, or 90%, e.g., as compared to an engineered mammalian cell that is substantially identical to or identical to the engineered mammalian cell except that it does not comprise a reduction in the function of an MHC class I component.

In an embodiment, the engineered mammalian cell described herein (e.g., ARPE-19), comprises a reduction in the level or function of HLA-A by about 0.05%, 0.1%, 0.5%, 0.75%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the level or function of HLA-A.

In an embodiment, the engineered mammalian cell described herein (e.g., ARPE-19), comprises a reduction in the level or function of HLA-A between 1-25%, 5-25%, 10-25%, 25-50%, 25-75%, 50-75%, or 75-100%, e.g., as compared to an engineered mammalian cell that is substantially identical to or identical to the engineered mammalian cell except that it does not comprise a reduction in the level or function of HLA-A. In an embodiment, the engineered mammalian cell described herein (e.g., ARPE-19), comprises a reduction in the level or function of HLA-A greater than about 50%, 75%, or 90%, e.g., as compared to an engineered mammalian cell that is substantially identical to or identical to the engineered mammalian cell except that it does not comprise a reduction in the level or function of HLA-A.

In an embodiment, the engineered mammalian cell described herein (e.g., ARPE-19), comprises a reduction in the level or function of HLA-B by about 0.05%, 0.1%, 0.5%, 0.75%, 1%, 2%, 3%, 4%, 5%, 6, 7% 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the level or function of HLA-B. In an embodiment, the engineered mammalian cell described herein (e.g., ARPE-19), comprises a reduction in the level or function of HLA-B between 1-25%, 5-25%, 10-25%, 25-50%, 25-75%, 50-75%, or 75-100%, e.g., as compared to an engineered mammalian cell that is substantially identical to or identical to the engineered mammalian cell except that it does not comprise a reduction in the level or function of HLA-B. In an embodiment, the engineered mammalian cell described herein (e.g., ARPE-19), comprises a reduction in the level or function of HLA-B greater than about 50%, 75%, or 90%, e.g., as compared to an engineered mammalian cell that is substantially identical to or identical to the engineered mammalian cell except that it does not comprise a reduction in the level or function of HLA-B.

In an embodiment, the engineered mammalian cell described herein (e.g., ARPE-19), comprises a reduction in the level or function of HLA-C by about 0.05%, 0.1%, 0.5%, 0.75%, 1%, 2%, 3%, 4%, 5%, 6%, 7%, 8%, 9%, 10, 1%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the level or function of HLA-C. In an embodiment, the engineered mammalian cell described herein (e.g., ARPE-19), comprises a reduction in the level or function of HLA-C between 1-25%, 5-25%, 10-25%, 25-50%, 25-75%, 50-75%, or 75-100%, e.g., as compared to an engineered mammalian cell that is substantially identical to or identical to the engineered mammalian cell except that it does not comprise a reduction in the level or function of HLA-C. In an embodiment, the engineered mammalian cell described herein (e.g., ARPE-19), comprises a reduction in the level or function of HLA-C greater than about 50%, 75%, or 90%, e.g., as compared to an engineered mammalian cell that is substantially identical to or identical to the engineered mammalian cell except that it does not comprise a reduction in the level or function of HLA-C.

In an embodiment, the engineered mammalian cell described herein (e.g., ARPE-19), comprises a reduction in the level or function of beta-2M by about 0.05%, 0.1%, 0.5%, 0.75%, 1%, 2%, 3%, 4%, 5%, 6, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the level or function of beta-2M. In an embodiment, the engineered mammalian cell described herein (e.g., ARPE-19), comprises a reduction in the level or function of beta-2M between 1-25%, 5-25%, 10-25%, 25-50%, 25-75%, 50-75%, or 75-100%, e.g., as compared to an engineered mammalian cell that is substantially identical to or identical to the engineered mammalian cell except that it does not comprise a reduction in the level or function of beta-2M. In an embodiment, the engineered mammalian cell described herein (e.g., ARPE-19), comprises a reduction in the level or function of beta-2M greater than about 50%, 75%, or 90%, e.g., as compared to an engineered mammalian cell that is substantially identical to or identical to the engineered mammalian cell except that it does not comprise a reduction in the level or function of beta-2M.

In some embodiments, the MHC class I protein complex or a component thereof, e.g., HLA-A, HLA-B, HLA-C, or beta-2M, is encoded by one or more nucleotide sequences, or fragments thereof, provided in Table 5.

In some embodiments, the reduction in the level or function of the MHC class I protein complex or a component thereof, e.g., HLA-A, HLA-B, HLA-C, or beta-2M persists for at least 15 minutes (e.g., 30 minutes, 1 hour, 12 hours, 24 hours, 48 hours, 72 hours, 1 week, 1 month, or 1 year). For example, in some embodiments, the reduction in the level or function of the MHC class I protein complex or a component thereof persists for at least 30 minutes. In some embodiments, the reduction in the level or function of the MHC class I protein complex or a component thereof persists for at least 1 hour. In some embodiments, the reduction in the level or function of the MHC class I protein complex or a component thereof persists for at least 12 hours. In some embodiments, the reduction in the level or function of the MHC class I protein complex or a component thereof persists for at least 24 hours. In some embodiments, the reduction in the level or function of the MHC class I protein complex or a component thereof persists for at least 48 hours. In some embodiments, the reduction in the level or function of the MHC class I protein complex or a component thereof persists for at least 72 hours. In some embodiments, the reduction in the level or function of the MHC class I protein complex or a component thereof persists for at least 1 week. In some embodiments, the reduction in the level or function of the MHC class I protein complex or a component thereof persists for at least 1 month. In some embodiments, the reduction in the level or function of the MHC class I protein complex or a component thereof persists for at least 1 year.

The MHC class II protein complex is a class of molecules present on the surface of antigen-presenting cells within a subject, such as dendritic cells, mononuclear phagocytes, certain endothelial cells, and B cells. One key distinguishing feature between the MHC class II protein complex and the MHC class I protein complex is that the antigens presented by the MHC class II protein complexes are derived from extracellular proteins, unlike the cytosolic antigens presented by the MHC class I protein complexes. Like the MHC class I protein complexes, the MHC class II protein complexes are heterodimeric proteins that consist of two polypeptide chains, the alpha-chain and the beta-chain. Unlike the MHC class I protein complexes, the MHC class II protein complex's alpha-chain and beta-chain comprises homogeneous peptides. The alpha-peptide comprises the alpha-1 and beta-1 domains, which come together to make a membrane-distal peptide-binding groove, while the beta-peptide comprises the alpha-2 and beta-2 domains, which form a membrane-proximal immunoglobulin-like domain. The peptide-binding groove is made up of two α-helices walls and a β sheet. Because the antigen-binding groove of MHC class II molecules is open at both ends while the corresponding groove on class I molecules is closed at each end, the antigens presented by MHC class II molecules are longer, generally between 15 and 24 amino acid residues long.

Exemplary MHC class II protein complex components include HLA-DP, HLA-DM, HLA-DOA, HLA-DOB, HLA-DQ, and HLA-DR. In some embodiments, the engineered mammalian cell (e.g., ARPE-19) comprises a reduction in a level or function of an MHC class II protein complex or a component thereof, e.g., HLA-DP, HLA-DM, HLA-DOA, HLA-DOB, HLA-DQ, or HLA-DR. In some embodiments, the engineered mammalian cell (e.g., ARPE-19) comprises a reduction in the alpha and/or beta chain of the MHC class II protein complex or component thereof. For example, in some embodiments, the engineered mammalian cell (e.g., ARPE-19) comprises a reduction in a level or function of the alpha-1 domain. In some embodiments, the level or function of the alpha-1 domain is reduced by about 10% (e.g., about 20%, 30%, 40%, 50%, 60%, 70%, 80%, 90%, or 100%). For example, in some embodiments, the level or function of the alpha-1 domain is reduced by about 20%. In some embodiments, the level or function of the alpha-1 domain is reduced by about 30%. In some embodiments, the level or function of the alpha-1 domain is reduced by about 40%. In some embodiments, the level or function of the alpha-1 domain is reduced by about 50%. In some embodiments, the level or function of the alpha-1 domain is reduced by about 60%. In some embodiments, the level or function of the alpha-1 domain is reduced by about 70%. In some embodiments, the level or function of the alpha-1 domain is reduced by about 80%. In some embodiments, the level or function of the alpha-1 domain is reduced by about 90%. In some embodiments, the level or function of the alpha-1 domain is reduced by about 100%.

In some embodiments, the level or function of the alpha-1 domain is reduced by at least 5% (e.g., at least 10%, 20%, 30%, 40%, 50%, 60%, 70%, 80%, 90%, 95%, 99%, 99.9% or more). For example, in some embodiments, the level or function of the alpha-1 domain is reduced by at least 10%. In some embodiments, the level or function of the alpha-1 domain is reduced by at least 20%. In some embodiments, the level or function of the alpha-1 domain is reduced by at least 30%. In some embodiments, the level or function of the alpha-1 domain is reduced by at least 40%. In some embodiments, the level or function of the alpha-1 domain is reduced by at least 50%. In some embodiments, the level or function of the alpha-1 domain is reduced by at least 60%. In some embodiments, the level or function of the alpha-1 domain is reduced by at least 70%. In some embodiments, the level or function of the alpha-1 domain is reduced by at least 80%. In some embodiments, the level or function of the alpha-1 domain is reduced by at least 90%. In some embodiments, the level or function of the alpha-1 domain is reduced by at least 95%. In some embodiments, the level or function of the alpha-1 domain is reduced by at least 99%. In some embodiments, the level or function of the alpha-1 domain is reduced by at least 99.9%. In some embodiments, the level or function of the alpha-1 domain is reduced by more than 99.9%.

In some embodiments, the engineered mammalian cell (e.g., ARPE-19) comprises a reduction in a level or function of the alpha-2 domain. In some embodiments, the level or function of the alpha-2 domain is reduced by about 10% (e.g., about 20%, 30%, 40%, 50%, 60%, 70%, 80%, 90%, or 100%). For example, in some embodiments, the level or function of the alpha-2 domain is reduced by about 20%. In some embodiments, the level or function of the alpha-2 domain is reduced by about 30%. In some embodiments, the level or function of the alpha-2 domain is reduced by about 40%. In some embodiments, the level or function of the alpha-2 domain is reduced by about 50%. In some embodiments, the level or function of the alpha-2 domain is reduced by about 60%. In some embodiments, the level or function of the alpha-2 domain is reduced by about 70%. In some embodiments, the level or function of the alpha-2 domain is reduced by about 80%. In some embodiments, the level or function of the alpha-2 domain is reduced by about 90%. In some embodiments, the level or function of the alpha-2 domain is reduced by about 100%.

In some embodiments, the level or function of the alpha-2 domain is reduced by at least 5% (e.g., at least 10%, 20%, 30%, 40%, 50%, 60%, 70%, 80%, 90%, 95%, 99%, 99.9% or more). For example, in some embodiments, the level or function of the alpha-2 domain is reduced by at least 10%. In some embodiments, the level or function of the alpha-2 domain is reduced by at least 20%. In some embodiments, the level or function of the alpha-2 domain is reduced by at least 30%. In some embodiments, the level or function of the alpha-2 domain is reduced by at least 40%. In some embodiments, the level or function of the alpha-2 domain is reduced by at least 50%. In some embodiments, the level or function of the alpha-2 domain is reduced by at least 60%. In some embodiments, the level or function of the alpha-2 domain is reduced by at least 70%. In some embodiments, the level or function of the alpha-2 domain is reduced by at least 80%. In some embodiments, the level or function of the alpha-2 domain is reduced by at least 90%. In some embodiments, the level or function of the alpha-2 domain is reduced by at least 95%. In some embodiments, the level or function of the alpha-2 domain is reduced by at least 99%. In some embodiments, the level or function of the alpha-2 domain is reduced by at least 99.9%. In some embodiments, the level or function of the alpha-2 domain is reduced by more than 99.9%.

In some embodiments, the engineered mammalian cell (e.g., ARPE-19) comprises a reduction in a level or function of the beta-1 domain. In some embodiments, the level or function of the beta-1 domain is reduced by about 10% (e.g., about 20%, 30%, 40%, 50%, 60%, 70%, 80%, 90%, or 100%). For example, in some embodiments, the level or function of the beta-1 domain is reduced by about 20%. In some embodiments, the level or function of the beta-1 domain is reduced by about 30%. In some embodiments, the level or function of the beta-1 domain is reduced by about 40%. In some embodiments, the level or function of the beta-1 domain is reduced by about 50%. In some embodiments, the level or function of the beta-1 domain is reduced by about 60%. In some embodiments, the level or function of the beta-1 domain is reduced by about 70%. In some embodiments, the level or function of the beta-1 domain is reduced by about 80%. In some embodiments, the level or function of the beta-1 domain is reduced by about 90%. In some embodiments, the level or function of the beta-1 domain is reduced by about 100%.

In some embodiments, the level or function of the beta-1 domain is reduced by at least 5% (e.g., at least 10%, 20%, 30%, 40%, 50%, 60%, 70%, 80%, 90%, 95%, 99%, 99.9% or more). For example, in some embodiments, the level or function of the beta-1 domain is reduced by at least 10%. In some embodiments, the level or function of the beta-1 domain is reduced by at least 20%. In some embodiments, the level or function of the beta-1 domain is reduced by at least 30%. In some embodiments, the level or function of the beta-1 domain is reduced by at least 40%. In some embodiments, the level or function of the beta-1 domain is reduced by at least 50%. In some embodiments, the level or function of the beta-1 domain is reduced by at least 60%. In some embodiments, the level or function of the beta-1 domain is reduced by at least 70%. In some embodiments, the level or function of the beta-1 domain is reduced by at least 80%. In some embodiments, the level or function of the beta-1 domain is reduced by at least 90%. In some embodiments, the level or function of the beta-1 domain is reduced by at least 95%. In some embodiments, the level or function of the beta-1 domain is reduced by at least 99%. In some embodiments, the level or function of the beta-1 domain is reduced by at least 99.9%. In some embodiments, the level or function of the beta-1 domain is reduced by more than 99.9%.

In some embodiments, the engineered mammalian cell (e.g., ARPE-19) comprises a reduction in a level or function of the beta-2 domain. In some embodiments, the level or function of the beta-2 domain is reduced by about 10% (e.g., about 20%, 30%, 40%, 50%, 60%, 70%, 80%, 90%, or 100%). For example, in some embodiments, the level or function of the beta-2 domain is reduced by about 20%. In some embodiments, the level or function of the beta-2 domain is reduced by about 30%. In some embodiments, the level or function of the beta-2 domain is reduced by about 40%. In some embodiments, the level or function of the beta-2 domain is reduced by about 50%. In some embodiments, the level or function of the beta-2 domain is reduced by about 60%. In some embodiments, the level or function of the beta-2 domain is reduced by about 70%. In some embodiments, the level or function of the beta-2 domain is reduced by about 80%. In some embodiments, the level or function of the beta-2 domain is reduced by about 90%. In some embodiments, the level or function of the beta-2 domain is reduced by about 100%.

In some embodiments, the level or function of the beta-2 domain is reduced by at least 5% (e.g., at least 10%, 20%, 30%, 40%, 50%, 60%, 70%, 80%, 90%, 95%, 99%, 99.9% or more). For example, in some embodiments, the level or function of the beta-2 domain is reduced by at least 10%. In some embodiments, the level or function of the beta-2 domain is reduced by at least 20%. In some embodiments, the level or function of the beta-2 domain is reduced by at least 30%. In some embodiments, the level or function of the beta-2 domain is reduced by at least 40%. In some embodiments, the level or function of the beta-2 domain is reduced by at least 50%. In some embodiments, the level or function of the beta-2 domain is reduced by at least 60%. In some embodiments, the level or function of the beta-2 domain is reduced by at least 70%. In some embodiments, the level or function of the beta-2 domain is reduced by at least 80%. In some embodiments, the level or function of the beta-2 domain is reduced by at least 90%. In some embodiments, the level or function of the beta-2 domain is reduced by at least 95%. In some embodiments, the level or function of the beta-2 domain is reduced by at least 99%. In some embodiments, the level or function of the beta-2 domain is reduced by at least 99.9%. In some embodiments, the level or function of the beta-2 domain is reduced by more than 99.9%.

In some embodiments, the reducing the level or function of substantially decreases, prevents, or inhibits, e.g., by 10%, 20%, 30%, 40%, 50%, 75%, 90%, 95%, or more, the interaction (e.g., binding) of the alpha-chain of an MHC class II protein complex or component thereof to the beta-chain. For example, in some embodiments, the reducing the level or function of substantially decreases, prevents, or inhibits the interaction (e.g., binding) of the alpha-1 domain to the alpha-2 or beta-2 domain. In some embodiments, the reducing the level or function of substantially decreases, prevents, or inhibits the interaction (e.g., binding) of the beta-1 domain to the alpha-2 or beta-2 domain. In some embodiments, the reducing the level or function of substantially decreases, prevents, or inhibits the interaction (e.g., binding) of the alpha-2 domain to the alpha-1 or beta-1 domain. In some embodiments, the reducing the level or function of substantially decreases, prevents, or inhibits the interaction (e.g., binding) of the beta-2 domain to the alpha-1 domain or beta-1 domain. In some embodiments, the reducing the level or function of substantially decreases, prevents, or inhibits the interaction (e.g., binding) of the alpha-1 domain to the beta-1 domain. In some embodiments, the reducing the level or function of substantially decreases, prevents, or inhibits the interaction (e.g., binding) of the alpha-2 domain to the beta-2 domain.

HLA-DP is a protein/peptide-antigen receptor and graft-versus-host disease antigen that is composed of 2 subunits, DPα and DPβ. DPα and DPβ are encoded by two loci, HLA-DPA1 and HLA-DPB1, that are found in the MHC Class II (or HLA-D) region in the HLA complex on human chromosome 6. HLA-DP is an αβ-heterodimer cell-surface receptor. Each DP subunit (α-subunit, β-subunit) is composed of a α-helical N-terminal domain, an IgG-like β-sheet, a membrane spanning domain, and a cytoplasmic domain. The α-helical domain forms the sides of the peptide binding groove. The β-sheet regions form the base of the binding groove and the bulk of the molecule as well as the inter-subunit (non-covalent) binding region. Peptide-bound HLA-DP complexes interact with TCRs on CD4+ T-cells.

In some embodiments, the reducing the level or function of substantially decreases, prevents, or inhibits, e.g., by 10%, 20%, 30%, 40%, 50%, 75%, 90%, 95%, or more, the interaction (e.g., binding) of HLA-DP to a CD4+ T-cell TCR and/or any peptide (e.g., an antigenic peptide) bound within its peptide-binding groove. For example, in some embodiments, the reducing the level or function of substantially decreases, prevents, or inhibits the interaction (e.g., binding) of HLA-DP to a CD4+ T-cell TCR. In some embodiments, the reducing the level or function of substantially decreases, prevents, or inhibits the interaction (e.g., binding) of HLA-DP to a peptide bound within its peptide-binding groove.

HLA-DM, a non-classical MHC molecule, is an intracellular protein involved in the mechanism of antigen presentation and is encoded by the genes HLA-DMA and HLA-DMB. Like HLA-DP, the genes for HLA-DM are located in the MHCII region of the human chromosome 6. HLA-DM is a molecular chaperone that works in lysosomes and endosomes in cells of the immune system. It works in APCs like macrophages, dendritic cells, and B cells by interacting with MHC class II molecules (Arndt et al (2000). “Functional HLA-DM on the surface of B cells and immature dendritic cells”. The EMBO Journal. 19 (6): 1241-51. doi:10.1093/emboj/19.6.1241; Pashine et al (2003). “Interaction of HLA-DR with an acidic face of HLA-DM disrupts sequence-dependent interactions with peptides”. Immunity. 19 (2): 183-92. doi:10.1016/S1074-7613(03)00200-0). HLA-DM protects the MHC class II molecules from breaking down, and regulates which proteins or peptides bind to them as well. This regulates how and when a peptide acts as an antigen initiating an immune response. HLA-DM is required to release CLIP (a fragment resulting from cathepsin S or cathepsin D-mediated cleavage of CD74) from MHC class II molecules, to chaperone empty MHC molecules against denaturation, to facilitate antigen-antigen exchange (e.g., by releasing weakly bound peptides from the peptide-binding groove in order to load peptides with higher-affinity binding), and to control proper loading and release of peptides at the peptide-binding groove. To release peptides from the MHC groove, HLA-DM binds to the N terminus of the groove, altering its conformation and breaking hydrogen bonds such that the peptide that was interacting with the MHC groove can no longer bind and is ejected (Yin et al. (2015). “Evaluating the Role of HLA-DM in MHC Class II-Peptide Association Reactions”. Journal of Immunology. 195 (2): 706-16. doi:10.4049/jimmunol.1403190). HLA-DM assists in catalysis of peptide exchange not only in late endosomes traveling from the ER, but also on cell membranes and in early endosomes. Much of this pathway is still being researched, but it is known that HLA-DM can load exogenous peptides onto MHC class II molecules when they are being expressed on cell surfaces. Loading can also occur in early endosomes that are quickly recycled. HLA-DM does not have the capacity to bind peptides due to its lack of a deep peptide-binding groove—instead, it contains a shallow, negatively charged indent with two disulfide bonds.

HLA-DM also interacts heavily with chaperone protein HLA-DO, another non-classical MHC molecule. HLA-DO starts binding to DM in early endosomes, but is expressed less in late endosomes/lysosomes. The binding between HLA-DM and HLA-DO is less strong at low pH, but overall much stronger than HLA-DM binding to MHC molecules. Both HILA-DM and HLA-DO lack a transport signal N-terminus. Before encountering an antigen, DO acts as a chaperone of DM to stabilize it against denaturation and direct it into lysosomes. It binds in the same location to HLA-DM as MHC class II molecules bind, thereby preventing HLA-DM from binding to MHC class II molecules. This inhibits peptide exchange catalysis and keeps CLIP in the MHC groove until antigen-containing lysosome fuses with DM/DO/MHC containing lysosomes, prompting the degradation of HLA-DO molecules. The alpha-chain of HLA-DO (HLA-DOA) is encoded by the HLA-DOA gene and the beta-chain (HLA-DOB) is encoded by the HLA-DOB gene.

In some embodiments, the reducing the level or function of substantially decreases, prevents, or inhibits, e.g., by 10%, 20%, 30%, 40%, 50%, 75%, 90%, 95%, or more, the interaction (e.g., binding) of HLA-DM to CLIP, HLA-DO, and the beta-chain of MHC class II molecules. For example, in some embodiments, the reducing the level or function of substantially decreases, prevents, or inhibits the interaction (e.g., binding) of HLA-DM to CLIP. In some embodiments, the reducing the level or function of substantially decreases, prevents, or inhibits the interaction (e.g., binding) of HLA-DM to HLA-DO. In some embodiments, the reducing the level or function of substantially decreases, prevents, or inhibits the interaction (e.g., binding) of HLA-DO to HLA-DM. In some embodiments, the reducing the level or function of substantially decreases, prevents, or inhibits the interaction (e.g., binding) of HLA-DM to the beta-chain of MHC class II molecules.

In some embodiments, the reducing the level or function of substantially decreases, prevents, or inhibits, e.g., by 10%, 20%, 30%, 40%, 50%, 75%, 90%, 95%, or more, the interaction (e.g., binding) of HLA-DO to HLA-DM or any peptide bound within its peptide-binding groove. In some embodiments, the reducing the level or function of substantially decreases, prevents, or inhibits the interaction (e.g., binding) of HLA-DO to HLA-DM. In some embodiments, the reducing the level or function of substantially decreases, prevents, or inhibits the interaction (e.g., binding) of HLA-DO to a peptide bound within its peptide-binding groove.

HLA-DQ is a cell surface receptor protein found on antigen-presenting cells. It is an ap heterodimer of type MHC class II. The alpha- and beta-chains are encoded by two loci, HLA-DQA1 and HLA-DQB1, that are adjacent to each other on chromosome band 6p21.3. Both the alpha-chain and the beta-chain comprise a plethora of variants. A person often produces two α-chain and two β-chain variants and thus 4 isoforms of HLA-DQ. The HLA-DQ loci are in close genetic linkage to HLA-DR, and less closely linked to HLA-DP, the non-classical MHC class II molecules (HLA-DM and HLA-DO), and the MHC class I molecules.

Different isoforms of HLA-DQ can bind to and present different antigens to T-cells. In this process T-cells are stimulated to grow and can signal B-cells to produce antibodies. HLA-DQ functions in recognizing and presenting foreign antigens (proteins derived from potential pathogens). For example, peptide-bound HLA-DP complexes interact with TCRs on CD4+ T-cells. HLA-DQ is also involved in recognizing common self-antigens and presenting those antigens to the immune system in order to develop tolerance from a very young age. When tolerance to self-proteins is lost, HLA-DQ may become involved in autoimmune disease. Two autoimmune diseases in which HLA-DQ is involved are coeliac disease and type 1 diabetes. HLA-DQ mediates autoimmunity by skewing the TCR repertoire during thymic selection (Rubio et al. (2021). “HLA class II mediates type 1 diabetes risk by anti-insulin repertoire selection”. bioRxiv: 2021.09.06.458974. doi:10.1101/2021.09.06.458974). Carriers of risk serotypes such as HLA-DQ8 have a higher proportion of circulating T-cell receptors that may bind insulin, the primary autoantigen in type 1 diabetes.

In some embodiments, the reducing the level or function of substantially decreases, prevents, or inhibits, e.g., by 10%, 20%, 30%, 40%, 50%, 75%, 90%, 95%, or more, the interaction (e.g., binding) of ILA-DQ to CD4+ T-cell TCRs and/or any peptide bound within its peptide-binding groove. For example, in some embodiments, the reducing the level or function of substantially decreases, prevents, or inhibits the interaction (e.g., binding) of HLA-DQ to a CD4+ T-cell TCR. In some embodiments, the reducing the level or function of substantially decreases, prevents, or inhibits the interaction (e.g., binding) of HLA-DQ to a peptide bound within its peptide-binding groove.

HLA-DR is an αβ heterodimer, cell surface receptor, each subunit of which contains two extracellular domains, a membrane-spanning domain and a cytoplasmic tail. Both α and β chains are anchored in the membrane. The N-terminal domain of the mature protein forms an alpha-helix that constitutes the exposed part of the binding groove, the C-terminal cytoplasmic region interacts with the other chain forming a beta-sheet under the binding groove spanning to the cell membrane. The majority of the peptide contact positions are in the first 80 residues of each chain. HLA-DR is encoded by several loci and several [symbol] enes [symbol] f different function at each locus. The DR α-chain is encoded by the HLA-DRA locus. Unlike the other DR loci, functional variation in mature DRA gene products is absent. The DR β-chain is encoded by 4 loci, however no more than 3 functional loci are present in a single individual, and no more than two on a single chromosome (Marsh et al (2010). “Nomenclature for factors of the HLA system, 2010”. Tissue Antigens. 75 (4): 291-455. doi:10.1111/j.1399-0039.2010.01466.x). Sometimes an individual may only possess 2 copies of the same locus, DRB1. The HLA-DRB1 locus is ubiquitous and encodes a very large number of functionally variable gene products (HLA-DR1 to HLA-DR17). The HLA-DRB3 locus encodes HLA-DR52, is moderately variable, and is variably associated with certain HLA-DRB1 types. The HLA-DRB4 locus encodes HLA-DR53, has some variation, and is associated with certain HLA-DRB1 types. The HLA-DRB5 locus encodes HLA-DR51, which is typically invariable and is linked to the HLA-DR2 types. HLA-DR interacts with CD74, HLA-DM, CD4+ T-cell TCRs, and/or any peptide bound within its peptide-binding groove.

In some embodiments, the reducing the level or function of substantially decreases, prevents, or inhibits, e.g., by 10%, 20%, 30%, 40%, 50%, 75%, 90%, 95%, or more, the interaction (e.g., binding) of HLA-DR to CD74, HLA-DM, CD4+ T-cell TCRs, and/or any peptide bound within its peptide-binding groove. For example, in some embodiments, the reducing the level or function of substantially decreases, prevents, or inhibits the interaction (e.g., binding) of HLA-DR to CD74. In some embodiments, the reducing the level or function of substantially decreases, prevents, or inhibits the interaction (e.g., binding) of HLA-DR to HLA-DM. In some embodiments, the reducing the level or function of substantially decreases, prevents, or inhibits the interaction (e.g., binding) of HLA-DR to a CD4+ T-cell TCR. In some embodiments, the reducing the level or function of substantially decreases, prevents, or inhibits the interaction (e.g., binding) of HLA-DR to a peptide bound within its peptide-binding groove.

In some embodiments, the reducing the level or function of the MHC class II protein complex or a component thereof, e.g., HLA-DP, HLA-DM, HLA-DOA, HLA-DOB, HLA-DQ, or HLA-DR, results in reduced antigen presentation, thereby reducing and/or abrogating the recruitment of immune cells, e.g., T cells and NK cells.

In some embodiments, the reducing the level or function of the MHC class II protein complex or a component thereof, e.g., HLA-DP, HLA-DM, HLA-DOA, HLA-DOB, ILA-DQ, or HLA-DR, comprises mutating one or more nucleotides in the nucleotide sequence of one or more genes selected from HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5. A nucleotide mutation may comprise a nucleotide deletion, addition, and/or substitution. Such a mutation, as described herein, may result in a reduction in expression of the gene, e.g., by reducing, altering, or abrogating the transcription and/or splicing of the nucleotide sequence. For example, in some embodiments, the reducing the level or function of the MHC class II protein complex or a component thereof comprises mutating one or more nucleotides in the nucleotide sequence of HLA-DP. In some embodiments, the reducing the level or function of the MHC class II protein complex or a component thereof comprises mutating one or more nucleotides in the nucleotide sequence of HLA-DM. In some embodiments, the reducing the level or function of the MHC class II protein complex or a component thereof comprises mutating one or more nucleotides in the nucleotide sequence of HLA-DOA. In some embodiments, the reducing the level or function of the MHC class II protein complex or a component thereof comprises mutating one or more nucleotides in the nucleotide sequence of HLA-DOB. In some embodiments, the reducing the level or function of the MHC class II protein complex or a component thereof comprises mutating one or more nucleotides in the nucleotide sequence of HLA-DP. In some embodiments, the reducing the level or function of the MHC class II protein complex or a component thereof comprises mutating one or more nucleotides in the nucleotide sequence of HLA-DM In some embodiments, the reducing the level or function of the MHC class II protein complex or a component thereof comprises mutating one or more nucleotides in the nucleotide sequence of HLA-DOA. In some embodiments, the reducing the level or function of the MHC class II protein complex or a component thereof comprises mutating one or more nucleotides in the nucleotide sequence of HLA-DOB. In some embodiments, the reducing the level or function of the MHC class II protein complex or a component thereof comprises mutating one or more nucleotides in the nucleotide sequence of HLA-DQA1. In some embodiments, the reducing the level or function of the MHC class II protein complex or a component thereof comprises mutating one or more nucleotides in the nucleotide sequence of HLA-DQB1. In some embodiments, the reducing the level or function of the MHC class II protein complex or a component thereof comprises mutating one or more nucleotides in the nucleotide sequence of HLA-DRA. In some embodiments, the reducing the level or function of the MHC class II protein complex or a component thereof comprises mutating one or more nucleotides in the nucleotide sequence of HLA-DRBL. In some embodiments, the reducing the level or function of the MHC class II protein complex or a component thereof comprises mutating one or more nucleotides in the nucleotide sequence of HLA-DRB3. In some embodiments, the reducing the level or function of the MHC class II protein complex or a component thereof comprises mutating one or more nucleotides in the nucleotide sequence of HLA-DRB4. In some embodiments, the reducing the level or function of the MHC class II protein complex or a component thereof comprises mutating one or more nucleotides in the nucleotide sequence of HLA-DRB5.

In some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a nucleotide sequence provided in Table 5. In some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a sequence having at least 60% (e.g., at least 65%, 70%, 75%, 80%, 85%, 90%, 95%, 99%, 99.9%, or greater) sequence identity to a nucleotide sequence provided in Table 5. For example, in some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a sequence having at least 65% sequence identity to a nucleotide sequence provided in Table 5. In some embodiments, the nucleotide sequence of the HLA-DP, HLA-DM, HLA-DOA, HLA-DOB, HLA-DOA, HLA-DQB1, HLA-DRA, HLA-DRB1, HLA-DRB3, HLA-DRB4, and HLA-DRB5 genes comprises a sequence having at least 70% sequence identity to a nucleotide sequence provided in Table 5. In some embodiments, the nucleotide sequence of the HLA-DP, HLA-DM, HLA-DOA, HLA-DOB, HLA-DOA1, HLA-DOB1, HLA-DRA, HLA-DRB1, HLA-DRB3, HLA-DRB4, and HLA-DRB5 genes comprises a sequence having at least 75% sequence identity to a nucleotide sequence provided in Table 5. In some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a sequence having at least 80% sequence identity to a nucleotide sequence provided in Table 5. In some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a sequence having at least 85% sequence identity to a nucleotide sequence provided in Table 5. In some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a sequence having at least 90% sequence identity to a nucleotide sequence provided in Table 5. In some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a sequence having at least 95% sequence identity to a nucleotide sequence provided in Table 5. In some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a sequence having at least 99% sequence identity to a nucleotide sequence provided in Table 5. In some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a sequence having at least 99.9% sequence identity to a nucleotide sequence provided in Table 5. In some embodiments, the nucleotide sequence of the HLA-DP, HLA-DM, HLA-DOA, HLA-DOB, HLA-DOA1, HLA-DQB1, HLA-DRA, HLA-DRB1, HLA-DRB3, HLA-DRB4, and HLA-DRB5 genes comprises a sequence having greater than 99.9% sequence identity to a nucleotide sequence provided in Table 5.

In some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a sequence having at least 60% (e.g., at least 65%, 70%, 75%, 80%, 85%, 90%, 95%, 99%, 99.9%, or greater) sequence homology to a nucleotide sequence provided in Table 5. For example, in some embodiments, the nucleotide sequence of the HLA-DP, HLA-DM, HLA-DOA, HLA-DOB, HLA-DOA1, HLA-DOB1, HLA-DRA, HLA-DRB1, HLA-DRB3, HLA-DRB4, and HLA-DRB5 genes comprises a sequence having at least 65% sequence homology to a nucleotide sequence provided in Table 5. In some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a sequence having at least 70% sequence homology to a nucleotide sequence provided in Table 5. In some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a sequence having at least 75% sequence homology to a nucleotide sequence provided in Table 5. In some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a sequence having at least 80% sequence homology to a nucleotide sequence provided in Table 5. In some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a sequence having at least 85% sequence homology to a nucleotide sequence provided in Table 5. In some embodiments, the nucleotide sequence of the HLA-DP, HLA-DM, HLA-DOA, HLA-DOB, HLA-DQA1, HLA-DOB1, HLA-DRA, HLA-DRB1, HLA-DRB3, HLA-DRB4, and HLA-DRB5 genes comprises a sequence having at least 90% sequence homology to a nucleotide sequence provided in Table 5.

In some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a sequence having at least 95% sequence homology to a nucleotide sequence provided in Table 5. In some embodiments, the nucleotide sequence of the HLA-DP, HLA-DM, HLA-DOA, HLA-DOB, HLA-DOA, HLA-DQB1, HLA-DRA, HLA-DRB1, HLA-DRB3, HLA-DRB4, and HLA-DRB5 genes comprises a sequence having at least 99% sequence homology to a nucleotide sequence provided in Table 5. In some embodiments, the nucleotide sequence of the HLA-DP, HLA-DM, HLA-DOA, HLA-DOB, HLA-DQA1, HLA-DQB1, HLA-DRA, HLA-DRB1, HLA-DRB3, HLA-DRB4, and HLA-DRB5 genes comprises a sequence having at least 99.9% sequence homology to a nucleotide sequence provided in Table 5. In some embodiments, the nucleotide sequence of the HLA-DP, HLA-DM, HLA-DOA, HLA-DOB, HLA-DOA1, HLA-DQB1, HLA-DRA, HLA-DRB1, HLA-DRB3, HLA-DRB4, and HLA-DRB5 genes comprises a sequence having greater than 99.9% sequence homology to a nucleotide sequence provided in Table 5.

In an embodiment, the engineered mammalian cell of the present disclosure comprises a reduced level or function in an MHC class II protein complex or a component thereof, e.g., HLA-DP, HLA-DM, HLA-DOA, HLA-DOB, HLA-DQ, or ILA-DR. In an embodiment, the engineered mammalian cell described herein (e.g., ARPE-19), comprises a reduction in the expression of a MHC class II component by about 0.05%, 0.1%, 0.5%, 0.75%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the level of an MHC class II component. In an embodiment, the engineered mammalian cell described herein (e.g., ARPE-19), comprises a reduction in the expression of a MHC class II component between 1-25%, 5-25%, 10-25%, 25-50%, 25-75%, 50-75%, or 75-100%, e.g., as compared to an engineered mammalian cell that is substantially identical to or identical to the engineered mammalian cell except that it does not comprise a reduction in the level of an MHC class II component. In an embodiment, the engineered mammalian cell described herein (e.g., ARPE-19), comprises a reduction in the expression of a MHC class II component greater than about 50%, 75%, or 90%, e.g., as compared to an engineered mammalian cell that is substantially identical to or identical to the engineered mammalian cell except that it does not comprise a reduction in the level of an MHC class II component.

In an embodiment, the engineered mammalian cell described herein (e.g., ARPE-19), comprises a reduction in the function of a MHC class II component by about 0.05%, 0.1%, 0.5%, 0.75%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the function of an MHC class II component. In an embodiment, the engineered mammalian cell described herein (e.g., ARPE-19), comprises a reduction in the function of a MHC class II component between 1-25%, 5-25%, 10-25%, 25-50%, 25-75%, 50-75%, or 75-100%, e.g., as compared to an engineered mammalian cell that is substantially identical to or identical to the engineered mammalian cell except that it does not comprise a reduction in the function of an MHC class II component. In an embodiment, the engineered mammalian cell described herein (e.g., ARPE-19), comprises a reduction in the function of a MHC class I component greater than about 50%, 75%, or 90%, e.g., as compared to an engineered mammalian cell that is substantially identical to or identical to the engineered mammalian cell except that it does not comprise a reduction in the function of an MHC class II component.

In some embodiments, the MHC class II protein complex or a component thereof, e.g., HLA-DP, HLA-DM, HLA-DOA, HLA-DOB, HLA-DQ, or HLA-DR, is encoded by one or more nucleotide sequences, or fragments thereof, provided in Table 5.

In some embodiments, the reduction in the level or function of the MHC class II protein complex or a component thereof persists for at least 15 minutes (e.g., 30 minutes, 1 hour, 12 hours, 24 hours, 48 hours, 72 hours, 1 week, 1 month, or 1 year). For example, in some embodiments, the reduction in the level or function of the MHC class II protein complex or a component thereof persists for at least 30 minutes. In some embodiments, the reduction in the level or function of the MHC class II protein complex or a component thereof persists for at least 1 hour. In some embodiments, the reduction in the level or function of the MHC class II protein complex or a component thereof persists for at least 12 hours. In some embodiments, the reduction in the level or function of the MHC class II protein complex or a component thereof persists for at least 24 hours. In some embodiments, the reduction in the level or function of the MHC class II protein complex or a component thereof persists for at least 48 hours. In some embodiments, the reduction in the level or function of the MHC class II protein complex or a component thereof persists for at least 72 hours. In some embodiments, the reduction in the level or function of the MHC class II protein complex or a component thereof persists for at least 1 week. In some embodiments, the reduction in the level or function of the MHC class II protein complex or a component thereof persists for at least 1 month. In some embodiments, the reduction in the level or function of the MHC class II protein complex or a component thereof persists for at least 1 year.

The class II major histocompatibility complex transactivator (CIITA) is a gene involved in the regulation of expression of MHC class II protein complexes. The CIITA gene is located on chromosome 16 that encodes the CIITA protein, which plays a role in enhancing the transcription of MHC class I genes. The CIITA protein comprises an acidic transcriptional activation domain, 4 leucine-rich repeats, and a GTP binding domain. The protein uses GTP binding to facilitate its own transport into the nucleus. Once in the nucleus, the protein acts as a positive regulator of class II major histocompatibility complex gene transcription, and is often referred to as the “master control factor” for the expression of these genes (Harton et al. (2000). “Class II transactivator: mastering the art of major histocompatibility complex expression”. Molecular and Cellular Biology. 20 (17): 6185-94. doi:10.1128/MCB.20.17.6185-6194.2000; LeibundGut-Landmann et al. (2004). “Mini-review: Specificity and expression of CIITA, the master regulator of MHC class II genes”. European Journal of Immunology. 34 (6): 1513-25. doi:10.1002/eji.200424964). CIITA expression is induced by interferon gamma (Heuberger et al. (2021). “Why do intestinal epithelial cells express MHC class II?”. Immunology. 162 (4): 357-367. doi:10.1111/imm.13270).

In an embodiment, the engineered mammalian cell of the present disclosure comprises a reduced level or function in of a CIITA protein. In some embodiments, the engineered mammalian cell (e.g., ARPE-19) comprises a reduction in a level or function of a domain of a CIITA protein, wherein the domain is selected from the transcriptional activation domain, a leucine-rich repeat domain, and a GTP binding domain. For example, in some embodiments, the engineered mammalian cell (e.g., ARPE-19) comprises a reduction in a level or function of the transcriptional activation domain. In some embodiments, the level or function of the transcriptional activation domain is reduced by about 10% (e.g., about 20%, 30%, 40%, 50%, 60%, 70%, 80%, 90%, or 100%). For example, in some embodiments, the level or function of the transcriptional activation domain is reduced by about 20%. In some embodiments, the level or function of the transcriptional activation domain is reduced by about 30%. In some embodiments, the level or function of the transcriptional activation domain is reduced by about 40%. In some embodiments, the level or function of the transcriptional activation domain is reduced by about 50%. In some embodiments, the level or function of the transcriptional activation domain is reduced by about 60%. In some embodiments, the level or function of the transcriptional activation domain is reduced by about 70%. In some embodiments, the level or function of the transcriptional activation domain is reduced by about 80%. In some embodiments, the level or function of the transcriptional activation domain is reduced by about 90%. In some embodiments, the level or function of the transcriptional activation domain is reduced by about 100%.

In some embodiments, the level or function of the transcriptional activation domain is reduced by at least 5% (e.g., at least 10%, 20%, 30%, 40%, 50%, 60%, 70%, 80%, 90%, 95%, 99%, 99.9% or more). For example, in some embodiments, the level or function of the transcriptional activation domain is reduced by at least 10%. In some embodiments, the level or function of the transcriptional activation domain is reduced by at least 20%. In some embodiments, the level or function of the transcriptional activation domain is reduced by at least 30%. In some embodiments, the level or function of the transcriptional activation domain is reduced by at least 40%. In some embodiments, the level or function of the transcriptional activation domain is reduced by at least 50%. In some embodiments, the level or function of the transcriptional activation domain is reduced by at least 60%. In some embodiments, the level or function of the transcriptional activation domain is reduced by at least 70%. In some embodiments, the level or function of the transcriptional activation domain is reduced by at least 80%. In some embodiments, the level or function of the transcriptional activation domain is reduced by at least 90%. In some embodiments, the level or function of the transcriptional activation domain is reduced by at least 95%. In some embodiments, the level or function of the transcriptional activation domain is reduced by at least 99%. In some embodiments, the level or function of the transcriptional activation domain is reduced by at least 99.9%. In some embodiments, the level or function of the transcriptional activation domain is reduced by more than 99.9%.

In some embodiments, the engineered mammalian cell (e.g., ARPE-19) comprises a reduction in a level or function of a leucine-rich repeat domain. In some embodiments, the level or function of the leucine-rich repeat domain is reduced by about 10% (e.g., about 20%, 30%, 40%, 50%, 60%, 70%, 80%, 90%, or 100%). For example, in some embodiments, the level or function of the leucine-rich repeat domain is reduced by about 20%. In some embodiments, the level or function of the leucine-rich repeat domain is reduced by about 30%. In some embodiments, the level or function of the leucine-rich repeat domain is reduced by about 40%. In some embodiments, the level or function of the leucine-rich repeat domain is reduced by about 50%. In some embodiments, the level or function of the leucine-rich repeat domain is reduced by about 60%. In some embodiments, the level or function of the leucine-rich repeat domain is reduced by about 70%. In some embodiments, the level or function of the leucine-rich repeat domain is reduced by about 80%. In some embodiments, the level or function of the leucine-rich repeat domain is reduced by about 90%. In some embodiments, the level or function of the leucine-rich repeat domain is reduced by about 100%.

In some embodiments, the level or function of the leucine-rich repeat domain is reduced by at least 5% (e.g., at least 10%, 20%, 30%, 40%, 50%, 60%, 70%, 80%, 90%, 95%, 99%, 99.9% or more). For example, in some embodiments, the level or function of the leucine-rich repeat domain is reduced by at least 10%. In some embodiments, the level or function of the leucine-rich repeat domain is reduced by at least 20%. In some embodiments, the level or function of the leucine-rich repeat domain is reduced by at least 30%. In some embodiments, the level or function of the leucine-rich repeat domain is reduced by at least 40%. In some embodiments, the level or function of the leucine-rich repeat domain is reduced by at least 50%. In some embodiments, the level or function of the leucine-rich repeat domain is reduced by at least 60%. In some embodiments, the level or function of the leucine-rich repeat domain is reduced by at least 70%.

In some embodiments, the level or function of the leucine-rich repeat domain is reduced by at least 80%. In some embodiments, the level or function of the leucine-rich repeat domain is reduced by at least 90%. In some embodiments, the level or function of the leucine-rich repeat domain is reduced by at least 95%. In some embodiments, the level or function of the leucine-rich repeat domain is reduced by at least 99%. In some embodiments, the level or function of the leucine-rich repeat domain is reduced by at least 99.9%. In some embodiments, the level or function of the leucine-rich repeat domain is reduced by more than 99.9%.

In some embodiments, the engineered mammalian cell (e.g., ARPE-19) comprises a reduction in a level or function of the GTP binding domain. In some embodiments, the level or function of the GTP binding domain is reduced by about 10% (e.g., about 20%, 30%, 40%, 50%, 60%, 70%, 80%, 90%, or 100%). For example, in some embodiments, the level or function of the GTP binding domain is reduced by about 20%. In some embodiments, the level or function of the GTP binding domain is reduced by about 30%. In some embodiments, the level or function of the GTP binding domain is reduced by about 40%. In some embodiments, the level or function of the GTP binding domain is reduced by about 50%. In some embodiments, the level or function of the GTP binding domain is reduced by about 60%. In some embodiments, the level or function of the GTP binding domain is reduced by about 70%. In some embodiments, the level or function of the GTP binding domain is reduced by about 80%. In some embodiments, the level or function of the GTP binding domain is reduced by about 90%. In some embodiments, the level or function of the GTP binding domain is reduced by about 100%.

In some embodiments, the level or function of the GTP binding domain is reduced by at least 5% (e.g., at least 10%, 20%, 30%, 40%, 50%, 60%, 70%, 80%, 90%, 95%, 99%, 99.9% or more). For example, in some embodiments, the level or function of the GTP binding domain is reduced by at least 10%. In some embodiments, the level or function of the GTP binding domain is reduced by at least 20%. In some embodiments, the level or function of the GTP binding domain is reduced by at least 30%. In some embodiments, the level or function of the GTP binding domain is reduced by at least 40%. In some embodiments, the level or function of the GTP binding domain is reduced by at least 50%. In some embodiments, the level or function of the GTP binding domain is reduced by at least 60%. In some embodiments, the level or function of the GTP binding domain is reduced by at least 70%. In some embodiments, the level or function of the GTP binding domain is reduced by at least 80%. In some embodiments, the level or function of the GTP binding domain is reduced by at least 90%. In some embodiments, the level or function of the GTP binding domain is reduced by at least 95%. In some embodiments, the level or function of the GTP binding domain is reduced by at least 99%. In some embodiments, the level or function of the GTP binding domain is reduced by at least 99.9%. In some embodiments, the level or function of the GTP binding domain is reduced by more than 99.9%.

In some embodiments, the reducing the level or function of substantially decreases, prevents, or inhibits, e.g., by 10%, 20%, 30%, 40%, 50%, 75%, 90%, 95%, or more, the interaction (e.g., binding) of a CIITA protein domain to another CIITA protein domain. For example, in some embodiments, the reducing the level or function of substantially decreases, prevents, or inhibits the interaction (e.g., binding) of the transcriptional activation domain to a leucine-rich repeat domain or the GTP binding domain. In some embodiments, the reducing the level or function of substantially decreases, prevents, or inhibits the interaction (e.g., binding) of a leucine-rich repeat domain to the transcriptional activation domain, a leucine-rich repeat domain, or the GTP binding domain. In some embodiments, the reducing the level or function of substantially decreases, prevents, or inhibits the interaction (e.g., binding) of the GTP binding domain to the transcriptional activation domain or a leucine-rich repeat domain.

The CIITA protein interacts with Mitogen-activated protein kinase 1 (MAPK1), nuclear receptor coactivator 1 (NCOA1), DNA-binding protein RFX5 (RFX5), DNA-binding protein RFXANK (RFXANK), exportin 1 (XPO1), and Zinc finger, X-linked, duplicated family member C (ZXDC). In some embodiments, the reducing the level or function of substantially decreases, prevents, or inhibits, e.g., by 10%, 20%, 30%, 40%, 50%, 75%, 90%, 95%, or more, the interaction (e.g., binding) of CIITA to MAPK1, NCOA1, RFX5, RFXANK, XPO1, and/or ZXDC. For example, in some embodiments, the reducing the level or function of substantially decreases, prevents, or inhibits the interaction (e.g., binding) of CIITA to MAPK1. In some embodiments, the reducing the level or function of substantially decreases, prevents, or inhibits the interaction (e.g., binding) of CIITA to NCOA1. In some embodiments, the reducing the level or function of substantially decreases, prevents, or inhibits the interaction (e.g., binding) of CIITA to RFX5. In some embodiments, the reducing the level or function of substantially decreases, prevents, or inhibits the interaction (e.g., binding) of CIITA to RFXANK. In some embodiments, the reducing the level or function of substantially decreases, prevents, or inhibits the interaction (e.g., binding) of CIITA to XPO1. In some embodiments, the reducing the level or function of substantially decreases, prevents, or inhibits the interaction (e.g., binding) of CIITA to ZXDC.

In some embodiments, the reducing the level or function of the CIITA protein results in the reduction of the level of expression (e.g., by reducing the level of transcription) of a MHC class II protein complex or a component thereof, e.g., HLA-DP, HLA-DM, HLA-DOA, HLA-DOB, ILA-DQ, and HLA-DR.

In some embodiments, the reducing the level or function of the CIITA protein comprises mutating one or more nucleotides in the nucleotide sequence of the CIITA gene. A nucleotide mutation may comprise a nucleotide deletion, addition, and/or substitution. Such a mutation, as described herein, may result in a reduction in expression of the gene, e.g., by reducing, altering, or abrogating the transcription and/or splicing of the nucleotide sequence.

In some embodiments, the nucleotide sequence of the CIITA gene comprises a nucleotide sequence provided in Table 5. In some embodiments, the nucleotide sequence of the CIITA genes comprises a sequence having at least 60% (e.g., at least 65%, 70%, 75%, 80%, 85%, 90%, 95%, 99%, 99.9%, or greater) sequence identity to a nucleotide sequence provided in Table 5. For example, in some embodiments, the nucleotide sequence of the CIITA genes comprises a sequence having at least 65% sequence identity to a nucleotide sequence provided in Table 5. In some embodiments, the nucleotide sequence of the CIITA genes comprises a sequence having at least 70% sequence identity to a nucleotide sequence provided in Table 5. In some embodiments, the nucleotide sequence of the CIITA genes comprises a sequence having at least 75% sequence identity to a nucleotide sequence provided in Table 5. In some embodiments, the nucleotide sequence of the CIITA genes comprises a sequence having at least 80% sequence identity to a nucleotide sequence provided in Table 5. In some embodiments, the nucleotide sequence of the CIITA genes comprises a sequence having at least 85% sequence identity to a nucleotide sequence provided in Table 5. In some embodiments, the nucleotide sequence of the CIITA genes comprises a sequence having at least 90% sequence identity to a nucleotide sequence provided in Table 5. In some embodiments, the nucleotide sequence of the CIITA genes comprises a sequence having at least 95% sequence identity to a nucleotide sequence provided in Table 5. In some embodiments, the nucleotide sequence of the CIITA genes comprises a sequence having at least 99% sequence identity to a nucleotide sequence provided in Table 5. In some embodiments, the nucleotide sequence of the CIITA genes comprises a sequence having at least 99.9% sequence identity to a nucleotide sequence provided in Table 5. In some embodiments, the nucleotide sequence of the CIITA genes comprises a sequence having greater than 99.9% sequence identity to a nucleotide sequence provided in Table 5.

In some embodiments, the nucleotide sequence of the CIITA genes comprises a sequence having at least 60% (e.g., at least 65%, 70%, 75%, 80%, 85%, 90%, 95%, 99%, 99.9%, or greater) sequence homology to a nucleotide sequence provided in Table 5. For example, in some embodiments, the nucleotide sequence of the CIITA genes comprises a sequence having at least 65% sequence homology to a nucleotide sequence provided in Table 5. In some embodiments, the nucleotide sequence of the CIITA genes comprises a sequence having at least 70% sequence homology to a nucleotide sequence provided in Table 5. In some embodiments, the nucleotide sequence of the CIITA genes comprises a sequence having at least 75% sequence homology to a nucleotide sequence provided in Table 5. In some embodiments, the nucleotide sequence of the CIITA genes comprises a sequence having at least 80% sequence homology to a nucleotide sequence provided in Table 5. In some embodiments, the nucleotide sequence of the CIITA genes comprises a sequence having at least 85% sequence homology to a nucleotide sequence provided in Table 5. In some embodiments, the nucleotide sequence of the CIITA genes comprises a sequence having at least 90% sequence homology to a nucleotide sequence provided in Table 5. In some embodiments, the nucleotide sequence of the CIITA genes comprises a sequence having at least 95% sequence homology to a nucleotide sequence provided in Table 5. In some embodiments, the nucleotide sequence of the CIITA genes comprises a sequence having at least 99% sequence homology to a nucleotide sequence provided in Table 5. In some embodiments, the nucleotide sequence of the CIITA genes comprises a sequence having at least 99.9% sequence homology to a nucleotide sequence provided in Table 5. In some embodiments, the nucleotide sequence of the CIITA genes comprises a sequence having greater than 99.9% sequence homology to a nucleotide sequence provided in Table 5.

In an embodiment, the engineered mammalian cell described herein (e.g., ARPE-19), comprises a reduction in the expression of CIITA by about 0.05%, 0.1%, 0.5%, 0.75%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the level of CIITA. In an embodiment, the engineered mammalian cell described herein (e.g., ARPE-19), comprises a reduction in the expression of CIITA between 1-25%, 5-25%, 10-25%, 25-50%, 25-75%, 50-75%, or 75-100%, e.g., as compared to an engineered mammalian cell that is substantially identical to or identical to the engineered mammalian cell except that it does not comprise a reduction in the level of CIITA. In an embodiment, the engineered mammalian cell described herein (e.g., ARPE-19), comprises a reduction in the expression of CIITA greater than about 50%, 75%, or 90%, e.g., as compared to an engineered mammalian cell that is substantially identical to or identical to the engineered mammalian cell except that it does not comprise a reduction in the level of CIITA.

In an embodiment, the engineered mammalian cell described herein (e.g., ARPE-19), comprises a reduction in the function of CIITA by about 0.05%, 0.1%, 0.5%, 0.75%, 1%, 2%, 3%, 4%, 5% 6% 7%, 8%, 9%, 10%, 15%, 20%, 25%, 30%, 35% 40% 45%, 5%, %55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the function of CIITA. In an embodiment, the engineered mammalian cell described herein (e.g., ARPE-19), comprises a reduction in the function of CIITA between 1-25%, 5-25%, 10-25%, 25-50%, 25-75%, 50-75%, or 75-100%, e.g., as compared to an engineered mammalian cell that is substantially identical to or identical to the engineered mammalian cell except that it does not comprise a reduction in the function of CIITA. In an embodiment, the engineered mammalian cell described herein (e.g., ARPE-19), comprises a reduction in the function of CIITA greater than about 50%, 75%, or 90%, e.g., as compared to an engineered mammalian cell that is substantially identical to or identical to the engineered mammalian cell except that it does not comprise a reduction in the function of CIITA.

In some embodiments, the CIITA is encoded by a nucleotide sequence, or a fragment thereof, provided in Table 5.

In some embodiments, the reduction in the level or function of CIITA persists for at least 15 minutes (e.g., 30 minutes, 1 hour, 12 hours, 24 hours, 48 hours, 72 hours, 1 week, 1 month, or 1 year). For example, in some embodiments, the reduction in the level or function of CIITA persists for at least 30 minutes. In some embodiments, the reduction in the level or function of CIITA persists for at least 1 hour. In some embodiments, the reduction in the level or function of CIITA persists for at least 12 hours. In some embodiments, the reduction in the level or function of CIITA persists for at least 24 hours. In some embodiments, the reduction in the level or function of CIITA persists for at least 48 hours. In some embodiments, the reduction in the level or function of CIITA persists for at least 72 hours. In some embodiments, the reduction in the level or function of CIITA persists for at least 1 week. In some embodiments, the reduction in the level or function of CIITA persists for at least 1 month. In some embodiments, the reduction in the level or function of CIITA persists for at least 1 year.

In an embodiment, the engineered mammalian cell described herein (e.g., ARPE-19), comprises a reduction in the level or function of a MHC class I protein complex and a reduction in the level or function of a MHC class II protein complex and/or CIITA. In an embodiment, the engineered mammalian cell described herein (e.g., ARPE-19), comprises a reduction in the level or function of HLA-A and a reduction in the level or function of a MHC class II protein complex and/or CIITA. In an embodiment, the engineered mammalian cell described herein (e.g., ARPE-19), comprises a reduction in the level or function of HLA-B and a reduction in the level or function of a MHC class II protein complex and/or CIITA. In an embodiment, the engineered mammalian cell described herein (e.g., ARPE-19), comprises a reduction in the level or function of HLA-C and a reduction in the level or function of a MHC class II protein complex and/or CIITA. In an embodiment, the engineered mammalian cell described herein (e.g., ARPE-19), comprises a reduction in the level or function of beta-2M and a reduction in the level or function of a MHC class II protein complex and/or CIITA.

TABLE 5
Exemplary sequences

| SEQ ID | | |
|---|---|---|
| NO.: | Gene | Sequence |
| 1 | HLA-A | AGATTCTCCCCAGACGCCGAGGATGGCCGTCATGGCGCCCC |
| | | GAACCCTCCTCCTGCTACTCTCGGGGGCCCTGGCCCTGACCC |
| | | AGACCTGGGCGGGTGAGTGCGGGGTCGGGAGGGAAACCGCC |
| | | TCTGCGGGGAGAAGCAAGGGGCCCTCCTGGCGGGGGCGCAG |
| | | GACCGGGGGAGCCGCGCCGGGACGAGGGTCGGGCAGGTCTC |
| | | AGCCACTGCTCGCCCCCAGGCTCCCACTCCATGAGGTATTTC |
| | | TTCACATCCGTGTCCCGGCCCGGCCGCGGGGAGCCCCGCTTC |
| | | ATCGCCGTGGGCTACGTGGACGACACGCAGTTCGTGCGGTTC |
| | | GACAGCGACGCCGCGAGCCAGAGGATGGAGCCGCGGGCGC |
| | | CGTGGATAGAGCAGGAGGGGCCGGAGTATTGGGACCAGGA |
| | | GACACGGAATGTGAAGGCCCAGTCACAGACTGACCGAGTGG |
| | | ACCTGGGGACCCTGCGCGGCTACTACAACCAGAGCGAGGCC |
| | | GGTGAGTGACCCCGGCCGGGGGCGCAGGTCAGGACCCCTCA |
| | | TCCCCCACGGACGGGCCAGGTCGCCCACAGTCTCCGGGTCC |
| | | GAGATCCACCCCGAAGCCGCGGGACCCCGAGACCCTTGCCC |
| | | CGGGAGAGGCCCAGGCGCCTTTACCCGGTTTCATTTTCAGTT |
| | | TAGGCCAAAAATCCCCCCGGGTTGGTCGGGGCTGGGCGGGG |
| | | CTCGGGGGACTGGGCTGACCGCGGGGTCGGGGCCAGGTTCT |
| | | CACACCATCCAGATAATGTATGGCTGCGACGTGGGGTCGGA |
| | | CGGGCGCTTCCTCCGCGGGTACCGGCAGGACGCCTACGACG |
| | | GCAAGGATTACATCGCCCTGAACGAGGACCTGCGCTCTTGG |
| | | ACCGCGGCGGACATGGCGGCTCAGATCACCAAGCGCAAGTG |
| | | GGAGGCGGCCCATGAGGCGGAGCAGTTGAGAGCCTACCTGG |
| | | ATGGCACGTGCGTGGAGTGGCTCCGCAGATACCTGGAGAAC |
| | | GGGAAGGAGACGCTGCAGCGCACGGGTACCAGGGGCCACG |
| | | GGGCGCCTCCCTGATCGCCTGTAGATCTCCCGGGCTGGCCTC |
| | | CCACAAGGAGGGGAGACAATTGGGACCAACACTAGAATATC |
| | | ACCCTCCCTCTGGTCCTGAGGGAGAGGAATCCTCCTGGGTTC |
| | | CAGATCCTGTACCAGAGAGTGACTCTGAGGTTCCGCCCTGCT |
| | | CTCTGACACAATTAAGGGATAAAATCTCTGAAGGAGTGACG |
| | | GGAAGACGATCCCTCGAATACTGATGAGTGGTTCCCTTTGAC |
| | | ACCGGCAGCAGCCTTGGGCCCGTGACTTTTCCTCTCAGGCCT |
| | | TGTTCTCTGCTTCACACTCAATGTGTGTGGGGGTCTGAGTCC |
| | | AGCACTTCTGAGTCCCTCAGCCTCCACTCAGGTCAGGACCAG |
| | | AAGTCGCTGTTCCCTTCTCAGGGAATAGAAGATTATCCCAGG |
| | | TGCCTGTGTCCAGGCTGGTGTCTGGGTTCTGTGCTCTCTTCCC |
| | | CATCCCGGGTGTCCTGTCCATTCTCAAGATGGCCACATGCGT |
| | | GCTGGTGGAGTGTCCCATGACAGATGCAAAATGCCTGAATTT |
| | | TCTGACTCTTCCCGTCAGACCCCCCCAAGACACATATGACCC |
| | | ACCACCCCATCTCTGACCATGAGGCCACCCTGAGGTGCTGGG |
| | | CCCTGGGCTTCTACCCTGCGGAGATCACACTGACCTGGCAGC |
| | | GGGATGGGGAGGACCAGACCCAGGACACGGAGCTCGTGGA |
| | | GACCAGGCCTGCAGGGGATGGAACCTTCCAGAAGTGGGCGG |
| | | CTGTGGTGGTGCCTTCTGGAGAGGAGCAGAGATACACCTGC |
| | | CATGTGCAGCATGAGGGTCTGCCCAAGCCCCTCACCCTGAG |
| | | ATGGGGTAAGGAGGGAGATGGGGGTGTCATGTCTCTTAGGG |
| | | AAAGCAGGAGCCTCTCTGGAGACCTTTAGCAGGGTCAGGGC |
| | | CCCTCACCTTCCCCTCTTTTCCCAGAGCTGTCTTCCCAGCCCA |
| | | CCATCCCCATCGTGGGCATCATTGCTGGCCTGGTTCTCCTTG |
| | | GAGCTGTGATCACTGGAGCTGTGGTCGCTGCCGTGATGTGGA |
| | | GGAGGAAGAGCTCAGGTGGAGAAGGGGTGAAGGGTGGGGT |
| | | CTGAGATTTCTTGTCTCACTGAGGGTTCCAAGCCCCAGCTAG |
| | | AAATGTGCCCTGTCTCATTACTGGGAAGCACCGTCCACAATC |
| | | ATGGGCCTACCCAGTCTGGGCCCTGTGTGCCAGCACTTACTC |
| | | TTTTGTAAAGCACCTGTTAAAATGAAGGACAGATTTATCACC |
| | | TTGATTACGGCGGTGATGGGACCTGATCCCAGCAGTCACAA |
| | | GTCACAGGGGAAGGTCCCTGAGGACAGACCTCAGGAGGGCT |
| | | ATTGGTCCAGGACCCACACCTGCTTTCTTCATGTTTCCTGATC |
| | | CCGCCCTGGGTCTGCAGTCACACATTTCTGGAAACTTCTCTG |
| | | GGGTCCAAGACTAGGAGGTTCCTCTAGGACCTTAAGGCCCT |
| | | GGCTCCTTTCTGGTATCTCACAGGACATTTTCTTCTCACAGAT |
| | | AGAAAAGGAGGGAGTTACACTCAGGCTGCAAGTAAGTATGA |
| | | AGGAGGCTGATGCCTGAGGTCCTTGGGATATTGTGTTTGGGA |
| | | GCCCATGGGGGAGCCCACCCACCTCACAATTCCTCCTCTAGC |
| | | CACATCTTCTGTGGGATCTGACCAGGTTCTGTTTTTGTTCTAC |
| | | CCCAGGCAGTGACAGTGCCCAGGGCTCTGATGTGTCCCTCAC |
| | | AGCTTGTAAAGGTGAGAGCTTGGAGGACCTAATGTGTGTTG |
| | | GGTGTTGGGCGGAACAGTGGACACAGCTGTGCTATGGGGTT |
| | | TCTTTGCATTGGATGTATTGAGCATGCGATGGGCTGTTTAAG |
| | | GTGTGACCCCTCACTGTGATGGATATGAATTTGTTCATGAAT |
| | | ATTTTTTTCTATAGTGTGAGACAGCTGCCTTGTGTGGGACTG |
| | | AGAGGCAAGAGTTGTTCCTGCCCTTCCCTTTGTGACTTGAAG |
| | | AACCCTGACTTTGTTTCTGCAAAGGCACCTGCATGTGTCTGT |
| | | GTTCGTGTAGGCATAATGTGAGGAGGTGGGGAGACCACCCC |
| | | ACCCCCATGTCCACCATGACCCTCTTCCCACGCTGACCTGTG |
| | | CTCCCTCCCCAATCATCTTTCCTGTTCCAGAGAGGTGGGGCT |
| | | GAGGTGTCTCCATCTCTGTCTCAACTTCATGGTGCACTGAGC |
| | | TGTAACTTCTTCCTTCCCTATTAAAATTAGAACCTTAGTATAA |
| | | ATTTACTTTCTCAAATTCTTGCCATGAGAGGTTGATGAGTTA |
| | | ATTAAAGGAGAAGATTCCTAAAATTTGAGAGACAAAATAAA |
| | | TGGAAGACATGAGAA |
| 2 | HLA-A | GCTCCCACTCCATGAGGTATTTCTTCACATCCGTGTCCCGGC |
| | | CCGGCCGCGGGGAGCCCCGCTTCATCGCCGTGGGCTACGTG |
| | | GACTACACGCAGTTCGTGCGGTTCGACAGCGACGCCGCGAG |
| | | CCAGAGGATGGAGCCGCGGGCGCCGTGGATAGAGCAGGAG |
| | | GGGCCGGAGTATTGGGACCAGGAGACACGGAATGTGAAGGC |
| | | CCAGTCACAGACTGACCGAGTGGACCTGGGGACCCTGCGCG |
| | | GCTACTACAACCAGAGCGAGGCCNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNGGTTCTCACAC |
| | | CATCCAGATAATGTATGGCTGCGACGTGGGGTCGGACGGGC |
| | | GCTTCCTCCGCGGGTACCGGCAGGACGCCTACGACGGCAAG |
| | | GATTACATCGCCCTGAACGAGGACCTGCGCTCTTGGACCGCG |
| | | GCGGACATGGCGGCTCAGATCACCAAGCGCAAGTGGGAGGC |
| | | GGCCCATGAGGCGGAGCAGTTGAGAGCCTACCTGGATGGCA |
| | | CGTGCGTGGAGTGGCTCCGCAGATACCTGGAGAACGGGAAG |
| | | GAGACGCTGCAGCGCACGG |
| 3 | HLA-A | GCTCCCACTCCATGAGGTATTTCTACACCTCCGTGTCCCGGC |
| | | CCGGCCGCGGGGAGCCCCGCTTCATCGCCGTGGGCTACGTG |
| | | GACGACACGCAGTTCGTGCGGTTCGACAGCGACGCCGCGAG |
| | | CCAGAGGATGGAGCCGCGGGCGCCGTGGATAGAGCAGGAG |
| | | GGGCCGGAGTATTGGGACCGGAACACACGGAAAGTGAAGG |
| | | CCCAGTCACAGACTGACCGAGTGGACCTGGGGACCCTGCGC |
| | | GGCTACTACAACCAGAGCGAGGACNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNGGTTCTCACA |
| | | CCATCCAGAGGATGTATGGCTGCGACGTGGGGCCGGACGGG |
| | | CGCTTCCTCCGCGGGTACCAGCAGGACGCTTACGACGGCAA |
| | | GGATTACATCGCCCTGAACGAGGACCTGCGCTCTTGGACCGC |
| | | GGCGGACATGGCGGCTCAGATCACCCAGCGCAAGTGGGAGA |
| | | CGGCCCATGAGGCGGAGCAGTGGAGAGCCTACCTGGAGGGC |
| | | GCGTGCGTGGAGTGGCTCCGCAGATACCTGGAGAACGGGAA |
| | | GGAGACGCTGCAGCGCACGG |
| 4 | HLA-A | GCTCCCACTCCATGAGGTATTTCTACACTTCCGTGTCCCGGC |
| | | CCGGCCGCGGGGAGCCCCGCTTCATCGCCGTGGGCTACGTG |
| | | GACGACACGCAGTTCGTGCGGTTCGACAGCGACGCCGCGAG |
| | | CCAGAGGATGGAGCCGCGGGCGCCGTGGATAGAGCAGGAG |
| | | GGGCCGGAGTATTGGGACCGGAACACACGGAATGTGAAGGC |
| | | CCAGTCACAGACTGACCGAGTGGACCTGGGGACCCTGCGCG |
| | | GCTACTACAACCAGAGCGAGGCCNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNGGTTCTCACAC |
| | | CATCCAGATGATGTATGGCTGCGACGTGGGGTCGGACGGGC |
| | | GCTTCCTCCGCGGGTACCGGCAGGACGCCTACGACGGCAAG |
| | | GATTACATCGCCCTGAAAGAGGACCTGCGCTCTTGGACCGC |
| | | GGCGGACATGGCAGCTCAGACCACCAAGCACAAGTGGGAGG |
| | | CGGCCCCTGTGGCGGAGCAGTGGAGAGCCTACCTGGAGGGC |
| | | ACGTGCGTGGAGTGGCTCCGCAGATACCTGGAGAACGGGAA |
| | | GGAGACGCTGCAGCGCACGG |
| 5 | HLA-A | GCTCTCACTCCATGAGGTATTTCTTCACATCCGTGTCCCGGC |
| | | CCGGCCGCGGGGAGCCCCGCTTCATCGCAGTGGGCTACGTG |
| | | GACGACACGCAGTTCGTGCGGTTCGACAGCGACGCCGCGAG |
| | | CCAGAGGATGGAGCCGCGGGCGCCGTGGATAGAGCAGGAG |
| | | GGTCCGGAGTATTGGGACGGGGAGACACGGAAAGTGAAGG |
| | | CCCACTCACAGACTCACCGAGTGGACCTGGGGACCCTGCGC |
| | | GGCTACTACAACCAGAGCGAGGCCNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNGGTTCTCACA |
| | | CCGTCCAGAGGATGTATGGCTGCGACGTGGGGTCGGACTGG |
| | | CGCTTCCTCCGCGGGTACCACCAGTACGCCTACGACGGCAA |
| | | GGATTACATCGCCCTGAAAGAGGACCTGCGCTCTTGGACCG |
| | | CGGCGGACATGGCAGCTCAGACCACCAAGCACAAGTGGGAG |
| | | GCGGCCCATGTGGCGGAGCAGTTGAGAGCCTACCTGGAGGG |
| | | CACGTGCGTGGAGTGGCTCCGCAGATACCTGGAGAACGGGA |
| | | AGGAGGCGCTGCAGCGCACGG |
| 6 | HLA-B | ATGCGGGTCACGGCGCCCCGAACCGTCCTCCTGCTGCTCTGG |
| | | GGGGCAGTGGCCCTGACCGAGACCTGGGCCGGCTCCCACTC |
| | | CATGAGGTATTTCTACACCGCCATGTCCCGGCCCGGCCGCGG |
| | | GGAGCCCCGCTTCATTGCAGTGGGCTACGTGGACGACACCC |
| | | AGTTCGTGAGGTTCGACAGCGACGCCGCGAGTCCGAGGACG |
| | | GAGCCCCGGGCGCCATGGATAGAGCAGGAGGGGCCGGAGT |
| | | ATTGGGACCGGAACACACAGATCTTCAAGACCAACACACAG |
| | | ACTTACCGAGAGAACCTGCGGATCGCGCTCCGCTACTACAA |
| | | CCAGAGCGAGGCCGGGTCTCACACTTGGCAGACGATGTATG |
| | | GCTGCGACGTGGGGCCGGACGGGCGCCTCCTCCGCGGGCAT |
| | | AACCAGTACGCCTACGACGGCAAAGATTACATCGCCCTGAA |
| | | CGAGGACCTGAGCTCCTGGACCGCGGCGGACACCGCGGCTC |
| | | AGATCACCCAGCGCAAGTGGGAGGCGGCCCGTGAGGCGGAG |
| | | CAGCTGAGAGCCTACCTGGAGGGCCTGTGCGTGGAGGGGCT |
| | | CCGCAGACACCTGGAGAACGGGAAGGAGACGCTGCAGCGC |
| | | GCGGACCCCCCAAAGACACACGTGACCCACCACCCCGTCTC |
| | | TGACCATGAGGCCACCCTGAGGTGCTGGGCCCTGGGCTTCTA |
| | | CCCTGCGGAGATCACACTGACCTGGCAGCGGGATGGCGAGG |
| | | ACCAAACTCAGGACACTGAGCTTGTGGAGACCAGACCAGCA |
| | | GGAGATAGAACCTTCCAGAAGTGGGCAGCTGTGGTGGTGCC |
| | | TTCTGGAGAAGAGCAGAGATACACATGCCATGTACAGCATG |
| | | AGGGGCTGCCGAAGCCCCTCACCCTGAGATGGGAGCCATCT |
| | | TCCCAGTCCACCATCCCCATCGTGGGCATTGTTGCTGGCCTG |
| | | GCTGTCCTAGCAGTTGTGGTCATCGGAGCTGTGGTCGCTACT |
| | | GTGATGTGTAGGAGGAAGAGCTCAGGTGGAAAAGGAGGGA |
| | | GCTACTCTCAGGCTGCGTCCAGCGACAGTGCCCAGGGCTCTG |
| | | ATGTGTCTCTCACAGCTTG |
| 7 | HLA-B | GCTCCCACTCCATGAGGTATTTCCACACCTCCGTGTCCCGGC |
| | | CCGGCCGCGGGGAGCCCCGCTTCATCACCGTGGGCTACGTG |
| | | GACGACACGCTGTTCGTGAGGTTCGACAGCGACGCCACGAG |
| | | TCCGAGGAAGGAGCCGCGGGCGCCATGGATAGAGCAGGAG |
| | | GGGCCGGAGTATTGGGACCGGGAGACACAGATCTCCAAGAC |
| | | CAACACACAGACTTACCGAGAGAGCCTGCGGAACCTGCGCG |
| | | GCTACTACAACCAGAGCGAGGCCGNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNGGTCTCAC |
| | | CCCCTCCAGAGCATGTACGGCTGCGACGTGGGGCCGGACGG |
| | | GCGCCTCCTCCGCGGGCATAACCAGTACGCCTACGACGGCA |
| | | AGGATTACATCGCCCTGAACGAGGACCTGCGCTCCTGGACC |
| | | GCCGCGGACACGGCGGCTCAGATCACCCAGCGCAAGTGGGA |
| | | GGCGGCCCGTGTGGCGGAGCAGCTGAGAGCCTACCTGGAGG |
| | | GCGAGTGCGTGGAGTGGCTCCGCAGATACCTGGAGAACGGG |
| | | AAGGAGACGCTGCAGCGCGCGG |
| 8 | HLA-B | TGGTGTAGGAGAAGAGGGATCAGGACGAAGTCCCATGTCCC |
| | | GGACGGGGCTCTCAGGGTCTCAGGCTCCGAGGGCCGCGTCT |
| | | GCATTGGGGAGGCGCAGCGTTGGGGATTCCCCACTCCCACG |
| | | AGTTTCACTTCTTCTCCCAACCTATGTCGGGTCCTTCTTCCAG |
| | | GATACTCGTGACGCGTCCCCATTTCCCACTCCCATTGGGTGT |
| | | CGGGTGTCTAGAGAAGCCAATCAGTGTCGCCGGGGTCCCAG |
| | | TTCTAAAGTCCCCACGCACCCACCCGGACTCAGAGTCTCCTC |
| | | AGACACCGAGATGCGGGTCACGGCACCCCGAACCCTCCTCC |
| | | TGCTGCTCTGGGGGGCCCTGGCCCTGACCGAGACCTGGGCC |
| | | GGTGAGTGCGGGTCGGGAGGGAAATGGCCTCTGTGGGGAGG |
| | | AGCGAGGGGACCGCAGGCGGGGGCGCAGGACCCGGGGAGC |
| | | CGCGCCGGGAGGAGGGTCTGGCGGGTCTCAGCCCCTCCTCG |
| | | CCCCCAGGCTCCCACTCCATGAGGTATTTCTACACCGCCATG |
| | | TCCCGGCCCGGCCGCGGGGAGCCCCGCTTCATCGCAGTGGG |
| | | CTACGTGGACGACACGCAGTTCGTGAGGTTCGACAGCGACG |
| | | CCGCGAGTCCGAGAGAGGAGCCGCGGGCGCCGTGGATAGAG |
| | | CAGGAGGGGCCGGAGTATTGGGACCGGAACACACAGATCT |
| | | ACAAGGCCCAGGCACAGACTGACCGAGAGAGCCTGCGGAAC |
| | | CTGCGCGGCTACTACAACCAGAGCGAGGCCGGTGAGTGACC |
| | | CCGGCCCGGGGCGCAGGTCACGACTCCCCATCCCCCACGTA |
| | | CGGCCCGGGTCGCCCCGAGTCTCCGGGTCCGAGATCCGCCTC |
| | | CCTGAGGCCGCGGGACCCGCCCAGACCCTCGACCGGCGAGA |
| | | GCCCCAGGCGCGTTTACCCGGTTTCATTTTCAGTTGAGGCCA |
| | | AAATCCCCGCGGGTTGGTCGGGGCGGGGCGGGGCTCGGGGG |
| | | ACGGGGCTGACCGCGGGGCCGGGGCCAGGGTCTCACACTTG |
| | | GCAGACGATGTATGGCTGCGACCTGGGGCCGGACGGGCGCC |
| | | TCCTCCGCGGGCATAACCAGTTAGCCTACGACGGCAAGGAT |
| | | TACATCGCCCTGAACGAGGACCTGAGCTCCTGGACCGCGGC |
| | | GGACACCGCGGCTCAGATCACCCAGCGCAAGTGGGAGGCGG |
| | | CCCGTGAGGCGGAGCAGCTGAGAGCCTACCTGGAGGGCACG |
| | | TGCGTGGAGTGGCTCCGCAGATACCTGGAGAACGGGAAGGA |
| | | GACGCTGCAGCGCGCGGGTACCAGGGGCAGTGGGGAGCCTT |
| | | CCCCATCTCCTATAGGTCGCCGGGGATGGCCTCCCACGAGAA |
| | | GAGGAGGAAAATGGGATCAGCGCTAGAATGTCGCCCTCCCT |
| | | TGAATGGAGAATGGCATGAGTTTTCCTGAGTTTCCTCTGAGG |
| | | GCCCCCTCTTCTCTCTAGGACAATTAAGGGATGACGTCTCTG |
| | | AGGAAATGGAGGGGAAGACAGTCCCTAGAATACTGATCAGG |
| | | GGTCCTCTTTGACCCCTGCAGCAGCCTTGGGAACCGTGACTT |
| | | TTCCTCTCAGGCCTTGTTCTCTGCCTCACACTCAGTGTGTTTG |
| | | GGGCTCTGATTCCAGCACTTCTGAGTCACTTTACCTCCACTC |
| | | AGATCAGGAGCAGAAGTCCCTGTTCCCCGCTCAGAGACTCG |
| | | AACTTTCCAATGAATAGGAGATTATCCCAGGTGCCTGCGTCC |
| | | AGGCTGGTGTCTGGGTTCTGTGCCCCTTCCCCACCCCAGGTG |
| | | TCCTGTCCATTCTCAGGCTGGTCACATGGGTGGTCCTAGGGT |
| | | GTCCTATGAGAGATGCAAAGCGCCTGAATTTTCTGACTCTTC |
| | | CCATCAGACCCCCCAAAGACACACGTGACCCACCACCCCAT |
| | | CTCTGACCATGAGGCCACCCTGAGGTGCTGGGCCCTGGGCTT |
| | | CTACCCTGCGGAGATCACACTGACCTGGCAGCGGGATGGCG |
| | | AGGACCAAACTCAGGACACTGAGCTTGTGGAGACCAGACCA |
| | | GCAGGAGATAGAACCTTCCAGAAGTGGGCAGCTGTGGTGGT |
| | | GCCTTCTGGAGAAGAGCAGAGATACACATGCCATGTACAGC |
| | | ATGAGGGGCTGCCGAAGCCCCTCACCCTGAGATGGGGTAAG |
| | | GAGGGGGATGAGGGGTCATATCTCTTCTCAGGGAAAGCAGG |
| | | AGCCCTTCTGGAGCCCTTCAGCAGGGTCAGGGCCCCTCGTCT |
| | | TCCCCTCCTTTCCCAGAGCCATCTTCCCAGTCCACCATCCCCA |
| | | TCGTGGGCATTGTTGCTGGCCTGGCTGTCCTAGCAGTTGTGG |
| | | TCATCGGAGCTGTGGTCGCTACTGTGATGTGTAGGAGGAAG |
| | | AGCTCAGGTAGGGAAGGGGTGAGGGGTGGGGTCTGGGTTTT |
| | | CTTGTCCCACTGGGGGTTTCAAGCCCCAGGTAGAAGTGTTCC |
| | | CTGCCTCATTACTGGGAAGCAGCATCCACACAGGGGCTAAT |
| | | GCAGCCTGGGACCCTGTGTGCCAGCACTTACTCTTTTGTGCA |
| | | GCACATGTGACAATGAAGGACGGATGTATCACCTTGATGGT |
| | | TGTGGTGTTGGGGTCCTGATTTCAGCATTCATGAGTCAGGGG |
| | | AAGGTCCCTGCTAAGGACAGACCTTAGGAGGGCAGTTGGTC |
| | | CAGGACCCACACTTGCTTTCCTCGTGTTTCCTGATCCTGCCTT |
| | | GGGTCTGTAGTCATACTTCTGGAAATTCCTTTTGGGTCCAAG |
| | | ACGAGGAGGTTCCTCTAAGATCTCATGGCCCTGCTTCCTCCC |
| | | AGTCCCCTCACAGNACATTTTCTTCCCACAGGTGGAAAAGGA |
| | | GGGAGCTACTCTCAGGCTGCGTGTAAGTGGTGGGGGTGGGA |
| | | GTGTGGAGGAGCTCACCCACCCCATAATTCCTCCTGTCCCAC |
| | | GTCTCCTGCGGGCTCTGACCAGGTCCTGTTTTTGTTCTACTCC |
| | | AGCCAGCGACAGTGCCCAGGGCTCTGATGTGTCTCTCACAGC |
| | | TTGAAAAGGTGAGATTCTTGGGGTCTAGAGTGGGCGGGGGG |
| | | GGGTGGGGTGGGGAGGGGGCAGAGGGGAAAGGCCTGGGTA |
| | | ATGGAGATTCTTTGATTGGGATGTTTCGCGTGTGTGGTGGGC |
| | | TGTTTAGAGTGTCATCACTTACCATGACTAACCAGAATTTGT |
| | | TCATGACTGTTGTTTTCTGTAGCCTGAGACAGCTGTCTTGTG |
| | | AGGGACTGAGATGCAGGATTTCTTCACTCCTCCCCTTTGTGA |
| | | CTTCAAGAGCCTCTGGCATCTCTTTCTGCAAAGGCACCTGAA |
| | | TGTGTCTGCGTCCCTGTTAGCATAATGTGAGGAGGTGGAGAG |
| | | ACAGCCCACCCCCGTGTCCACTGTGACCCCTGTTCGCATGCT |
| | | GACCTGTGTTTCCTCCCCAGAAGCTTGCGGAAGGGCGAATTC |
| | | CAGCACACTGGCGGCCGTTACTAGTGGATCCGAGCTCGGTA |
| | | CCAAGCTTGATGCA |
| 9 | HLA-B | GATCAGGACGAAGTCCCAGGTCCCGGACGGGGCTCTCAGGG |
| | | TCTCAGGCTCCGAGGGCCGCGTCTGCAATGGGGAGGCGCAG |
| | | CGTTGGGGATTCCCCACTCCCCTGAGTTTCACTTCTTCTCCCA |
| | | ACTTGTGTCGGGTCCTTCTTCCAGGATACTCGTGACGCATCC |
| | | CCACTTCCCACTCCCATTGGGTGTCGGATATCTAGAGAAGCC |
| | | AATCAGCGTCGCCGGGGTCCCAGTTCTAAAGTCCCCACGCAC |
| | | CCACCCGGACTCAGAGTCTCCTCAGACGCCAAGATGCTGGTC |
| | | ATGGCGCCCCGAACCGTCCTCCTGCTGCTCTCGGCGGCCCTG |
| | | GCCCTGACCGAGACCTGGGCCGGTGAGTGCGGGTCGGGAGG |
| | | GAAATGGCCTCTGCCGGGAGGAGCGAGGGGACCGCAGGCG |
| | | GGGGCGCAGGACCTGAGGAGCCGCGCCGGGAGGAGGGTCG |
| | | GGCGGGTTTCAGCCCCTCCTCGCCCCCAGGCTCCCACTCCAT |
| | | GAGGTATTTCTACACCTCCGTGTCCCGGCCCGGCCGCGGGGA |
| | | GCCCCGCTTCATCTCAGTGGGCTACGTGGACGACACGCAGTT |
| | | CGTGAGGTTCGACAGCGACGCCGCGAGTCCGAGAGAGGAGC |
| | | CGCGGGCGCCGTGGATAGAGCAGGAGGGGCCGGAATATTGG |
| | | GACCGGAACACACAGATCTGCAAGACCAACACACAGACTGA |
| | | CCGAGAGAGCCTGCGGAACCTGCGCGGCTACTACAACCAGA |
| | | GCGAGGCCGGTGAGTGACCCCGGCCCGGGGCGCAGGTCACG |
| | | ACTCCCCATCCCCCACGGACGGCCCGGGTCGCCCCGAGTCTC |
| | | CGGGTCCGAGATCCGCCTCCCTGAGGCCGCGGGACCCGCCC |
| | | AGACCCTCGACCGGCGAGAGCCCCAGGCGCGTTTACCCGGT |
| | | TTCATTTTCAGTTGAGGCCAAAATCCCCGCGGGTTGGTCGGG |
| | | GCGGGGCGGGGCTCGGGGGGACTGGGCTGACCGCGGGGGC |
| | | GGGGCCAGGGTCTCACACCCTCCAGTGGATGTATGGCTGCG |
| | | ACGTGGGGCCGGACGGGCGCCTCCTCCGCGGGTATAACCAG |
| | | TTCGCCTACGACGGCAAGGATTACATCGCCCTGAACGAGGA |
| | | CCTGAGCTCCTGGACCGCGGCGGACACCGCGGCTCAGATCA |
| | | CCCAGCGCAAGTGGGAGGCGGCCCGTGAGGCGGAGCAGCG |
| | | GAGAGCCTACCTGGAGGGCACGTGCGTGGAGTGGCTCCGCA |
| | | GACACCTGGAGAACGGGAAGGAGACGCTGCAGCGCGCGGG |
| | | TACCAGGGGCAGTGGGGAGCCTTCCCCATCTCCTATAGGTCG |
| | | CCGGGGATGGCCTCCCACGAGAAGAAGAGGAAAATGGGATC |
| | | AGCGCTAGAATGTCGCCCTCCCTTGAATGGAGAATGGCATG |
| | | AGTTTTCCTGAGTTTCCTCTGAGGGCCCCCTCTTCTCTCTAGG |
| | | ACAATTAAGGGATGACGTCTCTGAGGAAATGGAGGGGAAGA |
| | | CAGTCCCTAGAATACTGATCAGGGGTCCCCTTTGACCCCTGC |
| | | AGCAGCCTTGGGAACCATGACTTTTCTTCTCAGGCCTTGTTC |
| | | TCTGCCTCACACTCAGTGTGTTTGGGGCTCTGATTCCAGCAC |
| | | TTCTGAGTCACTTTACCTCCACTCAGATCAGGAGCAGAAGTC |
| | | TCTGTTCCCCGCTCAGAGACTCGAACTTTCCAATGAATAGAT |
| | | TATCCCAGGTGCCTGCGTCCAGGCTGGTGTCTGGGTTCTGTG |
| | | TCCCTTCCCCACCCCAGGTGTCCTGTCCATTCTCAGGCTGGTC |
| | | ACATGGGTGGTCCTAGGGTGTCCCATGAGAGATGCAAAGCG |
| | | CCTGAATTTTCTGACTCTTCCCATCAGACCCCCCAAAGACAC |
| | | ATGTGACCCACCACCCCATCTCTGACCATGAGGCCACCCTGA |
| | | GGTGCTGGGCCCTGGGCTTCTACCCTGCGGAGATCACACTGA |
| | | CCTGGCAGCGGGATGGCGAGGACCAAACTCAGGACACCGAG |
| | | CTTGTGGAGACCAGACCAGCAGGAGACAGAACCTTCCAGAA |
| | | GTGGGCAGCTGTGGTGGTGCCTTCTGGAGAAGAGCAGAGAT |
| | | ACACATGCCATGTACAGCATGAGGGGCTGCCGAAGCCCCTC |
| | | ACCCTGAGATGGGGTAAGGAGGGGGATGAGGGGTCATATCT |
| | | CTTCTCAGGGAAAGCAGGAGCCCTTCAGCAGGGTCAGGGCC |
| | | CCTCATCTTCCCCTCCTTTCCCAGAGCCATCTTCCCAGTCCAC |
| | | CGTCCCCATCGTGGGCATTGTTGCTGGCCTGGCTGTCCTAGC |
| | | AGTTGTGGTCATCGGAGCTGTGGTCGCTGCTGTGATGTGTAG |
| | | GAGGAAGAGTTCAGGTAGGGAAGGGGTGAGGGGTGGGGTC |
| | | TGGGTTTTCTTGTCCCACTGGGGGTTTCAAGCCCCAGGTAGA |
| | | AGTGTTCCCTGCATCATTACTGGGAAGCAGCATGCACACAG |
| | | GGGCTAACGCAGCCTGGGACCCTGTGTGCCAGCACTTACTCT |
| | | TTTGTGCAGCACATGTGACAATGAAGGACGGATGTATCACCT |
| | | TGATGGTTGTGGTGTTGGGGTCCTGATTCCAGCATTCATGAG |
| | | TCAGGGGAAGGTCCCTGCTAAGGACAGACCTTAGGAGGGCA |
| | | GTTGGTCCAGGACCCACACTTGCTTTCCTCGTGTTTCCTGATC |
| | | CTGCCCTGGGTCTGTAGTCATACTTCTGGAAATTCCTTTTGG |
| | | GTCCAAGACTAGGAGGTTCCTCTAAGATCTCATGGCCCTGCT |
| | | TCCTCCCAGTCCCCTCACAGGACATTTTCTTCCCACAGGTGG |
| | | AAAAGGAGGGAGCTACTCTCAGGCTGCGTGTAAGTGGTGGG |
| | | GGTGGGAGTGTGGAGGAGCTCACCCACCCCATAATTCCTCCT |
| | | GTCCCACGTCTCCTGTGGGCTCTGACCAGGTCCTGTTTTTGTT |
| | | CTACTCCAGCCAGCGACAGTGCCCAGGGCTCTGATGTGTCTC |
| | | TCACAGCTTGAAAAGGTGAGATTCTTGGGGTCTAGAGTGGG |
| | | CGGGGGGGGGGGGAGGGGGCAGNGGGGAAAGGCCTGGGTA |
| | | ATGGAGATTCTTTGATTGGGATGTTTCGCGTGTGTGATGGGC |
| | | TGTTCAGAGTGTCATCACTTACCATGACTAACCAGAATTTGT |
| | | TCATGACTGTTGTTTTCTGTAGCCTGAGACAGCTGTCTTGTG |
| | | AGGGACTGAGATGCAGGATTTCTTCACGCCTCCCCTTTGTGA |
| | | CTTCAAGAGCCTCTGGCATCTCTTTCTGCAAAGGCACCTGAA |
| | | TGTGTCTGCGTCCCTGTTAGCATAATGTGAGGAGGTGGAGAG |
| | | ACAGCCCACCCTTGTGTCCACTGTGACCCCTGTTCCCATGCT |
| | | GACCTGTGTTTCCTCCCCAGTCATCTTTCTTGTTCCAGAGAGG |
| | | TGGGGCTGGATGTCTCCATCTCTGTCTCAACTTTATATGCACT |
| | | GAGCTGCAACTTCTTACTTCCCTACTGAAAATAAGAATCTGA |
| | | ATATAAATTTGTTTTCTCAAATATTTGCTATGAGAGGTTGAT |
| | | GGATTAATTAAATAAGTCAATTCCTGGAATTTGAGAGAGCA |
| | | AATAAAGACCTGAGAACCTTCCAGAATCTGCATGTTCGCTGT |
| | | GCTGAGTCTGTTGCAGGTGGGGTGTGGAGAAGGCTGTGGGG |
| | | GGCCAAGTGTGGACGGGGCCTGTGCCCATTTGGTGTTGAGTC |
| | | CATCATGGGCTTTATGTGGTTAGTCCTCAGTTGGGTCACCTT |
| | | CACTGCTCCATTGTCCTTGTCCCTTCAGTGGAAACTTGTCCA |
| | | GTGGGAGCTGTGACCACAGAGGCTCACACATCGCCCAGGGC |
| | | GGCCCCTGCACACGGGGGTCTCTGTGCATTCTGAGACAAATT |
| | | TTCAGAGCCATTCACCTCCTGCCCTGCTTCTAGAGCTCCTTTT |
| | | CTGCTCTGCTCTTCTGCCCTCTCTCCCTGCCCTGGTTCTAGTG |
| | | ATCTTGGTGCTGAATCCAATCCCAACTCATGAATCTGTAAAG |
| | | CAGAGTCTAATTTAGACTTACATTTGTCTGTGAAATTGGACC |
| | | CATCATCAAGGACTGTTCTTTCCTGAAGAGAGAACCTGATTG |
| | | TGTGCTGCAGTGTGCTGGGGCAGGGGGTGCGG |
| 10 | HLA-B | ATGCGGGTCACGGCGCCCCGAACCGTCCTCCTGCTGCTCTGG |
| | | GGGGCAGTGGCCCTGACCGAGACCTGGGCCGGTGAGTGCGG |
| | | GGTCGGGAGGGAAATGGCCTCTGTGGGGAGGAGCGAGGGG |
| | | ACCGCAGGCGGGGGCGCAGGACCTGAGGAGCCGCGCCGGG |
| | | AGGAGGGTCGGGCGGGTCTCAGCCCCTCCTCGCCCCCAGGC |
| | | TCCCACTCCATGAGGTATTTCTACACCGCCATGTCCCGGCCC |
| | | GGCCGCGGGGAGCCCCGCTTCATCGCAGTGGGCTACGTGGA |
| | | CGACACCCAGTTCGTGAGGTTCGACAGCGACGCCGCGAGTC |
| | | CGAGGACGGAGCCCCGGGCGCCATGGATAGAGCAGGAGGG |
| | | GCCGGAGTATTGGGACGGGGAGACGCGGAACATGAAGGCCT |
| | | CCGCGCAGACTTACCGAGAGAACCTGCGGATCGCGCTCCGC |
| | | TACTACAACCAGAGCGAGGCCGGTGAGTGACCCCGGCCCGG |
| | | GGCGCAGGTCACGACTCCCCATCCCCCACGTACGGCCCGGG |
| | | TCGCCCCGAGTCTCCGGGTCCGAGATCCGCCTCCCTGAGGCC |
| | | GCGGGACCCGCCCAGACCCTCGACCGGCGAGAGCCCCAGGC |
| | | GCGTTTACCCGGTTTCATTTTCAGTTGAGGCCAAAATCCCCG |
| | | CGGGTTGGTCGGGGCGGGGCGGGGCTCGGGGGACGGGGCTG |
| | | ACCGCGGGGCCGGGGCCAGGGTCTCACATCATCCAGAGGAT |
| | | GTATGGCTGCGACCTGGGGCCCGACGGGCGCCTCCTCCGCG |
| | | GGCATGACCAGTCCGCCTACGACGGCAAGGATTACATCGCC |
| | | CTGAACGAGGACCTGAGCTCCTGGACCGCGGCGGACACCGC |
| | | GGCTCAGATCACCCAGCGCAAGTGGGAGGCGGCCCGTGTGG |
| | | CGGAGCAGCTGAGAGCCTACCTGGAGGGCCTGTGCGTGGAG |
| | | TGGCTCCGCAGATACCTGGAGAACGGGAAGGAGACGCTGCA |
| | | GCGCGCGGGTACCAGGGGCAGTGGGGAGCCTTCCCCATCTC |
| | | CTATAGGTCGCCGGGGATGGCCTCCCACGAGAAGAGGAGGA |
| | | AAATGGGATCAGCGCTAGAATGTCGCCCTCCCTTGAATGGA |
| | | GAATGGCATGAGTTTTCCTGAGTTTCCTCTGAGGGCCCCCTC |
| | | TTCTCTCTAGGACAATTAAGGGATGACGTCTCTGAGGAAATG |
| | | GAGGGGAAGACAGTCCCTAGAATACTGATCAGGGGTCCCCT |
| | | TTGACCCCTGCAGCAGCCTTGGGAACCGTGACTTTTCCTCTC |
| | | AGGCCTTGTTCTCTGCCTCACACTCAGTGTGTTTGGGGCTCT |
| | | GATTCCAGCACTTCTGAGTCACTTTACCTCCACTCAGATCAG |
| | | GAGCAGAAGTCCCTGTTCCCCGCTCAGAGACTCGAACTTTCC |
| | | AATGAATAGGAGATTATCCCAGGTGCCTGCGTCCAGGCTGG |
| | | TGTCTGGGTTCTGTGCCCCTTCCCCACACCAGGTGTCCTGTCC |
| | | ATTCTCAGGCTGGTCACATGGGTGGTCCTAGGGTGTCCCATG |
| | | AGAGATGCAAAGCGCCTGAATTTTCTGACTCTTCCCATCAGA |
| | | CCCCCCAAAGACACACGTGACCCACCACCCCGTCTCTGACCA |
| | | TGAGGCCACCCTGAGGTGCTGGGCCCTGGGCTTCTACCCTGC |
| | | GGAGATCACACTGACCTGGCAGCGGGATGGCGAGGACCAAA |
| | | CTCAGGACACTGAGCTTGTGGAGACCAGACCAGCAGGAGAT |
| | | AGAACCTTCCAGAAGTGGGCAGCTGTGGTGGTGCCTTCTGG |
| | | AGAAGAGCAGAGATACACATGCCATGTACAGCATGAGGGGC |
| | | TGCCGAAGCCCCTCACCCTGAGATGGGGTAAGGAGGGGGAT |
| | | GAGGGGTCATATCTCTTCTCAGGGAAAGCAGGAGCCCTTCTG |
| | | GAGCCCTTCAGCAGGGTCAGGGCCCCTCGTCTTCCCCTCCTT |
| | | TCCCAGAGCCATCTTCCCAGTCCACCATCCCCATCGTGGGCA |
| | | TTGTTGCTGGCCTGGCTGTCCTAGCAGTTGTGGTCATCGGAG |
| | | CTGTGGTCGCTACTGTGATGTGTAGGAGGAAGAGCTCAG |
| 11 | HLA-C | ATGCGGGTCATGGCGCCCCGAACCCTCCTCCTGCTGCTCTCG |
| | | GGAGCCCTGGCCCTGACCGAGACCTGGGCCTNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNGCTCCCACTCCATGAGGTATTTCTACACC |
| | | GCTGTGTCCCGGCCCGGCCGCGGAGAGCCCCACTTCATCGCA |
| | | GTGGGCTACGTGGACGACACGCAGTTCGTGCGGTTCGACAG |
| | | CGACGCCGCGAGTCCAAGAGGGGAGCCGCGGGCGCCGTGGG |
| | | TGGAGCAGGAGGGGCCGGAGTATTGGGACCGGGAGACACA |
| | | GAACTACAAGCGCCAGGCACAGACTGACCGAGTGAACCTGC |
| | | GGAAACTGCGCGGCTACTACAACCAGAGCGAGGCCGNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNGGTCTCACATCATCCAGAGGATGT |
| | | ATGGCTGCGACCTGGGGCCCGACGGGCGCCTCCTCCGCGGG |
| | | CATGACCAGTTAGCCTACGACGGCAAGGATTACATCGCCCT |
| | | GAACGAGGACCTGCGCTCCTGGACCGCCGCGGACACGGCGG |
| | | CTCAGATCACCCAGCGCAAGTGGGAAGCGGCCCGTGAGGCG |
| | | GAGCAGCTGAGAGCCTACCTGGAGGGCACGTGCGTGGAGTG |
| | | GCTCCGCAGATACCTGGAGAACGGGAAGGAGACGCTGCAGC |
| | | GCGCGGNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNAACACCCAAAGA |
| | | CACACGTGACCCACCATCCCGTCTCTGACCATGAGGCCACCC |
| | | TGAGGTGCTGGGCCCTGGGCTTCTACCCTGCGGAGATCACAC |
| | | TGACCTGGCAGCGGGATGGCGAGGACCAAACTCAGGACACC |
| | | GAGCTTGTGGAGACCAGGCCAGCAGGAGATGGAACCTTCCA |
| | | GAAGTGGGCAGCTGTGGTGGTGCCTTCTGGAGAAGAGCAGA |
| | | GATACACGTGCCATGTGCAGCACGAGGGGCTGCCGGAGCCC |
| | | CTCACCCTGAGATGGGNNNNNNNNNNNNNNNNNNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNAG |
| | | CCATCTTCCCAGCCCACCATCCCCATCGTGGGCATCGTTGCT |
| | | GGCCTGGCTGTCCTGGCTGTCCTAGCTGTCCTAGGAGCTGTG |
| | | ATGGCTGTTGTGATGTGTAGGAGGAAGAGCTCAGNNNNNNN |
| | | NNNNNNNNNNNNNNNNNNNNNNNNNNNNNNNNNNNNNNN |
| | | NNNNNNNNNNNNNNNNNNNNNNNNNNNNNNNNNNNNNNN |
| | | NNNNNNNNNNNNNNNGTGGAAAAGGAGGGAGCTGCTCTCA |
| | | GGCTGCGTNNNNNNNNNNCCAGCAACAGTGCCCAGGGCTCT |
| | | GATGAGTCTCTCATCGCTTGTAAAGNNNNNNNNNNCCTGA |
| 12 | HLA-C | AGGAGAAGAGGGATCAGGACGAAGTCCCAGGTCCCGGGCG |
| | | GGGCTCTCAGGGTCTCAGGCTCCAAGGGCCGTGTCTGCACTG |
| | | GGGAGGCGCCGCGTTGAGGATTCTCCACTCCCCTGAGTTTCA |
| | | CTTCTTCTCCCAACCTGCGTCGGGTCCTTCTTCCTGAATACTC |
| | | ATGACGCGTCCCCAATTCCCACTCCCATTGGGTGTCGGGTTC |
| | | TAGAGAAGCCAATCAGCGTCTCCGCAGTCCCGGTTCTAAAGT |
| | | CCCCAGTCACCCACCCGGACTCGGATTCTCCCCAGACGCCGA |
| | | GATGCGGGTCATGGCGCCCCGAACCCTCATCCTGCTGCTCTC |
| | | GGGAGCCCTGGCCCTGACCGAGACCTGGGCCTGTGAGTGCG |
| | | GGGTTGGGAGGGAAACGGCCTCTGCGGAGAGGAGCGAGGG |
| | | GCCCGCCCGGCGAGGGCGCAGGACCCGGGGAGCCGCGCAG |
| | | GGAGGAGGGTCGGGCGGGTCTCAGCCCCTCCTCGCCCCCAG |
| | | GCTCCCACTCCATGAGGTATTTCTACACCGCCGTGTCCCGGC |
| | | CCGGCCGCGGAGAGCCCCGCTTCATCGCAGTGGGCTACGTG |
| | | GACGACACGCAGTTCGTGCGGTTCGACAGCGACGCCGCGAG |
| | | TCCAAGAGGGGAGCCGCGGGCGCCGTGGGTGGAGCAGGAG |
| | | GGGCCGGAGTATTGGGACCGGGAGACACAGAAGTACAAGC |
| | | GCCAGGCACAGACTGACCGAGTGAACCTGCGGAAACTGCGC |
| | | GGCTACTACAACCAGAGCGAGGCCGGTGAGTGACCCCGGCC |
| | | CGGGGCGCAGGTCACGACCCCTCCCCATCCCCCACGGACGG |
| | | CCCGGGTCGCCCCGAGTCTCCCGGTCTGAGATCCACCCCGAG |
| | | GCTGCGGAACCCGCCCAGACCCTCGGCCGGAGAGAGCCCCA |
| | | GTCACCTTTACCCGGTTTCATTTTCAGTTTAGGCCAAAATCCC |
| | | CGCGGGTTGGTCGGGGCTGGGGCGGGGCTCGCGGGACGGGG |
| | | CTGACCACGGGGGCGGGGCCAGGGTCTCACACCCTCCAGTG |
| | | GATGTATGGCTGCGACCTGGGGCCCGACGGGCGCCTCCTCC |
| | | GCGGGTATGACCAGTCCGCCTACGACGGCAAGGATTACATC |
| | | GCCCTGAACGAGGACCTGCGCTCCTGGACCGCCGCGGACAC |
| | | GGCGGCTCAGATCACCCAGCGCAAGTGGGAGGCGGCCCGTG |
| | | CGGCGGAGCAGCAGAGAGCCTACCTGGAGGGCACGTGCGTG |
| | | GAGTGGCTCCGCAGATACCTGGAGAACGGGAAGGAGACGCT |
| | | GCAGCGCGCGGGTACCAGGGGCAGTGGGGAGCCTTCCCCAT |
| | | CTCCTGTAGATCTCCCGGGATGGCCTCCCACGAGGAGGGGA |
| | | GGAAAATGGGATCAGCGCTAGAATATCGCCCTCCCTTGAAT |
| | | GGAGAATGGGATGAGTTTTCCTGAGTTTCCTCTGAGGGCCCC |
| | | CTCTGCTCTCTAGGACAATTAAGGGATGAAGTCCTTGAGGAA |
| | | ATGGAGGGGAAGACAGTCCCTGGAATACTGATCAGGGGTCC |
| | | CCTTTGACCACTTTGACCACTGCAGCAGCTGTGGTCAGGCTG |
| | | CTGACCTTTCTCTCAGGCCTTGTTCTCTGCCTCATGCTCAATG |
| | | TGTTTGAAGGTTTGATTCCAGCTTTTCTGAGTTCTTCAGCCTC |
| | | CACTCAGGTCAGGACCAGAAGTCGCTGTTCCTCCCTCAGAGA |
| | | CTAGAACTTTCCAATGAATAGGAGATTATCCCAGGTGCCTGT |
| | | GTCCAGGCTGGCGTCTGGGTTCTGTGCCCCCTTCCCCACCCC |
| | | AGGTGTCCTGTCCATTCTCAGGATGGTCACATGGGCGCTGTT |
| | | GGAGTGTCGCAAGAGAGATACAAAGTGTCTGAATTTTCTGA |
| | | CTCTTCCCGTCAGAACACCCAAAGACACACGTGACCCACCAT |
| | | CTCGTCTCTGACCATGAGGCCACCCTGAGGTGCTGGGCCCTG |
| | | GGCTTCTACCCTGCGGAGATCACACTGACCTGGCAGCGGGA |
| | | TGGCGAGGACCAAACTCAGGACACCGAGCTTGTGGAGACCA |
| | | GGCCAGCAGGAGATGGAACCTTCCAGAAGTGGGCAGCTGTG |
| | | GTGGTGCCTTCTGGAGAAGAGCAGAGATACACGTGCCATGT |
| | | GCAGCACGAGGGGCTGCCGGAGCCCCTCACCCTGAGATGGG |
| | | GTAAGGAGGGGGATGAGGGGTCATGTGTCTTCTCAGGGAAA |
| | | GCAGAAGTCCTGGAGCCCTTCAGCCGGGTCAGGGCTGAGGC |
| | | TTGGGGGTCAGGGCCCCTCACCTTCCCCTCCTTTCCCAGAGC |
| | | CATCTTCCCAGCCCACCATCCCCATCGTGGGCATCGTTGCTG |
| | | GCCTGGCTGTCCTGGCTGTCCTAGCTGTCCTAGGAGCTGTGG |
| | | TGGCTGTTGTTATGTGTAGGAGGAAGAGCTCAGGTAGGGAA |
| | | GGGGTGAGGAGTGGGGTCTGGGTTTTCTTGTCCCACTGGGAG |
| | | TTTCAAGCCCCAGGTAGAAGTGTGCCCCACCTCGTTACTGGA |
| | | AGCACCATCCACACATGGGCCATCCCAGCCTGGGACCCTGT |
| | | GTGCCAGCACTTACTCTGTTGTGAAGCACATGACAATGAAG |
| | | GACAGATGTATCACCTTGATGATTATGGTGTTGGGGTCCTTG |
| | | ATTCCAGCATTCATGAGTCAGGGGAAGGTCCCTGCTAAGGA |
| | | CAGACCTTAGGAGGGCAGTTGCTCCAGAACCCACAGCTGCT |
| | | TTCCCTGTGTTTCCTGATCCTGCCCTGGGTCTGCAGTCATAGT |
| | | TCTGGAAACTTCTCTTGGGTCCAAGACTAGGAGGTTCCCCTA |
| | | AGATCGCATGGCCCTGCCTCCTCCCTGTCCCCTCACAGGGCA |
| | | TTTTCTTCCCACAGGTGGAAAAGGAGGGAGCTGCTCTCAGGC |
| | | TGCGTGTAAGTGATGGCAGTGGGCGTGTGGAGGAGCTCACC |
| | | CACCCCATAATTCCTCTTGTCCCACATCTCCTGCGGGCTCTG |
| | | ACCAGGTCTTTTTTTTTGTTCTACCCCAGCCAGCAACAGTGC |
| | | CCAGGGCTCTGATGAGTCTCTCATCGCTTGTAAAGGTGAGAT |
| | | TCTGGGGAGCTGAAGTGGTCGGGGGTGGGGCAGAGGGAAA |
| | | AGGCCTAGGTAATGGGGATCCTTTGATTGGGACGTTTCGAAT |
| | | GTGTGGTGAGCTGTTCAGAGTGTCATCACTTACCATGACTGA |
| | | CCTGAATTTGTTCATGACTATTGTGTTCTGTAGCCTGAGACA |
| | | GCTGCCTGTGTGGGACTGAGATGCAGGATTTCTTCACACCTC |
| | | TCCTTTGTGACTTCAAGAGCCTCTGGCATCTCTTTCTGCAAA |
| | | GGCACCTGAATGCGTCTGCGTTCCTGTTAGCATAATGTGAGG |
| | | AGGTGGAGAGACAGCCCACCCCCGTGTCCAC |
| 13 | HLA-C | AGGAGAAGAGGGATCAGGACGAAGTCCCAGGTCCCGGGCG |
| | | GGGCTCTCAGGGTCTCAGGCTCCAAGGGCCGTGTCTGCACTG |
| | | GGGAGGCGCCGCGTTGAGGATTCTCCACTCCCCTGAGTTTCA |
| | | CTTCTTCTCCCAACCTGCGACGGGTCCTTCTTCCTGAATACTC |
| | | ATGACGCGTCCCCAATTCCCACTCCCATTGGGTGTCGGGTTC |
| | | TAGAGAAGCCAATCAGCGTCTCCGCAGTCCCGGTTCTAAAGT |
| | | CCCCAGTCACCCACCCGGACTCGGATTCTCCCCAGACGCCGA |
| | | GATGCGGGTCATGGCGCCCCGAACCCTCCTCCTGCTGCTCTC |
| | | GGGAGCCCTGGCCCTGACCGAGACCTGGGCCTGTGAGTGCG |
| | | GGGTTGGGAGGGAAACGGCCTCTGCGGAGAGGAGCGAGGG |
| | | GCCCGCCCGGCGAGGGCGCAGGACCCGGGGAGCCGCGCAG |
| | | GGAGGAGGGTCGGGCGGGTCTCAGCCCCTCCTCGCCCCCAG |
| | | GCTCCCACTCCATGAGGTATTTCTACACCGCTGTGTCCCGGC |
| | | CCGGCCGCGGAGAGCCCCACTTCATCGCAGTGGGCTACGTG |
| | | GACGACACGCAGTTCGTGCGGTTCGACAGCGACGCCGCGAG |
| | | TCCAAGAGGGGAGCCGCGGGCGCCGTGGGTGGAGCAGGAG |
| | | GGGCCGGAGTATTGGGACCGGGAGACACAGAACTACAAGCG |
| | | CCAGGCACAGACTGACCGAGTGAACCTGCGGAAACTGCGCG |
| | | GCTACTACAACCAGAGCGAGGCCGGTGAGTGACCCCGGCCC |
| | | GGGGCGCAGGTCACGACCCCTCCCCATCCCCCACGGACGGC |
| | | CCGGGTCGCCCCGAGTCTCCCGGTCTGAGATCCACCCCGAGG |
| | | CTGCGGAACCCGCCCAGACCCTCGACCGGAGAGAGCCCCAG |
| | | TCACCTTTACCCGGTTTCATTTTCAGTTTAGGCCAAAATCCCC |
| | | GCGGGTTGGTCGGGGCTGGGGCGGGGCTCGGGGGACGGGGC |
| | | TGACCACGGGGGCGGGGCCAGGGTCTCACATCATCCAGAGG |
| | | ATGTATGGCTGCGACCTGGGGCCCGACGGGCGCCTCCTCCGC |
| | | GGGCATGACCAGTTAGCCTACGACGGCAAGGATTACATCGC |
| | | CCTGAACGAGGACCTGCGCTCCTGGACCGCCGCGGACACGG |
| | | CGGCTCAGATCACCCAGCGCAAGTGGGAGGCGGCCCGTGAG |
| | | GCGGAGCAGCTGAGAGCCTACCTGGAGGGCACGTGCGTGGA |
| | | GTGGCTCCGCAGATACCTGGAGAACGGGAAGGAGACGCTGC |
| | | AGCGCGCGGGTACCAGGGGCAGTGGGGAGCCTTCCCTATCT |
| | | CCTGTAGATCTCCCGGGATGGCCTTCCACGAGGAGGGGAGG |
| | | AAAATGGGATCAGCGCTAGAATATCGCCCTCCCTTGAATGG |
| | | AGAATGGGATGAGTTTTCCTGAGTTTCCTCTGAGGGCCCCCT |
| | | CTGCTCTCTAGGACAATTAAGGGATGAAGTCCTTGAGGAAA |
| | | TGGAGGGGAAGACAGTCCCTGGAATACTGATCAGGGGTCCC |
| | | CTTTGACCACTTTGACCACTGCAGCAGCTGTGGTCAGGCTGC |
| | | TGACCTTTCTCTCAGGCCTTGTTCTCTGCCTCACGCTCAATGT |
| | | GTTTGAAGGTTTGATTCCAGCTTTTCTGAGTCCTTCGGCCTCC |
| | | ACTCAGGTCAGGACCAGAAGTCGCTGTTCCTCCCTCAGAGAC |
| | | TAGAACTTTCCAATGAATAGGAGATTATCCCAGGTGCCTGTG |
| | | TCCAGGCTGGCGTCTGGGTTCTGTGCCCCCTTCCCCACCCCA |
| | | GGTGTCCTGTCCATTCTCAGGATGGTCACATGGGCGCTGTTG |
| | | GAGTGTCGCAAGAGAGATACAAAGTGTCTGAATTTTCTGACT |
| | | CTTCCCGTCAGAACACCCAAAGACACACGTGACCCACCATC |
| | | CCGTCTCTGACCATGAGGCCACCCTGAGGTGCTGGGCCCTGG |
| | | GCTTCTACCCTGCGGAGATCACACTGACCTGGCAGCGGGAT |
| | | GGCGAGGACCAAACTCAGGACACCGAGCTTGTGGAGACCAG |
| | | GCCAGCAGGAGATGGAACCTTCCAGAAGTGGGCAGCTGTGG |
| | | TGGTGCCTTCTGGAGAAGAGCAGAGATACACGTGCCATGTG |
| | | CAGCACGAGGGGCTGCCGGAGCCCCTCACCCTGAGATGGGG |
| | | TAAGGAGGGGGATGAGGGGTCATGTGTCTTCTCAGGGAAAG |
| | | CAGAAGTCCTGGAGCCCTTCAGCTGGGTCAGGGCTGAGGCT |
| | | TGGGGGTCAGGGCCCCTCACCTTCCCCTCCTTTCCCAGAGCC |
| | | ATCTTCCCAGCCCACCATCCCCATCGTGGGCATCGTTGCTGG |
| | | CCTGGCTGTCCTGGCTGTCCTAGCTGTCCTAGGAGCTGTGAT |
| | | GGCTGTTGTGATGTGTAGGAGGAAGAGCTCAGGTAGGGAAG |
| | | GGGTGAGGAGTGGGGTCTGGGTTTTCTTGTCCCACTGGGAGT |
| | | TTCAAGCCCCAGGTAGAAGTGTGCCCCACCTCGTTACTGGAA |
| | | GCACCATCCACACATGGGCCATCCCAGCCTGGGACCCTGTGT |
| | | GCCAGCACTTACTCTGTTGTGAAGCACATGACAATGAAGGA |
| | | CAGATGTATCACCTTGATGATTATGGTGTTGGGGTCCTTGAT |
| | | TCCAGCATTCATGAGTCAGGGGAAGGTCCCTGCTAAGGACA |
| | | GACCTTAGGAGGGCAGTTGCTCCAGAACCCACAGCTGCTTTC |
| | | CCCGTGTTTCCTGATCCTGCCCTGGGTCTGCAGTCATAGTTCT |
| | | GGAAACTTCTCTTGGGTCCAAGACTAGGAGGTTCCCCTAAGA |
| | | TCGCATGGCCCTGCCTCCTCCCTGTCCCCTCACAGGGCATTTT |
| | | CTTCCCACAGGTGGAAAAGGAGGGAGCTGCTCTCAGGCTGC |
| | | GTGTAAGTGATGGCGGTGGGCGTGTGGAGGAGCTTACCCAC |
| | | CCCATAATTCCTCTTGTCCCACATCTCCTGCAGGCTCTGACC |
| | | AGGTCTTTTTTTTTGTTCTACCCCAGCCAGCAACAGTGCCCA |
| | | GGGCTCTGATGAGTCTCTCATCGCTTGTAAAGGTGAGATTCT |
| | | GGGGAGCTGAAGTGGTCGGGGGTGGGGCAGAGGGAAAAGG |
| | | CCTAGGTAATGGGGATCCTTTGATTGGGACGTTTCGAATGTG |
| | | TGGTGAGCTGTTCAGAGTGTCATCACTTACCATGACTGACCT |
| | | GAATTTGTTCATGACTATTGTGTTCTGTAGCCTGAGACAGCT |
| | | GCCTGTGTGGGACTGAGATGCAGGATTTCTTCACACCTCTCC |
| | | TTTGTGACTTCAAGAGCCTCTGGCATCTCTTTCTGCAAAGGC |
| | | ATCTGAATGTGTCTGCGTTCCTGTTAGCATAATGTGAGGAGG |
| | | TGGAGAGACAGCCCACCCCCGTGTCCAC |
| 14 | HLA-C | AGGAGAAGAGGGATCAGGACGAAGTCCCAGGTCCCGGGCG |
| | | GGGCTCTCAGGGTCTCAGGCTCCAAGGGCCGTGTCTGCATTG |
| | | GGGAGGCGCCGCGTTGGGGATTCTCCACTCCCCTGAGTTTCA |
| | | CTTCTCCCAACCTGCGTCGGGTCCTTCTTCCTGAATACTCATG |
| | | ACGCGTCCCCAATTCCCACTCCCATTGGGTGTCGGGTTCTAG |
| | | AGAAGCCAATCAGCGTCTCCGCAGTCCCGGTTCTAAAGTCCC |
| | | CAGTCACCCACCCGGACTCACATTCTCCCCAGAGGCCGAGAT |
| | | GCGGGTCATGGCGCCCCGAGCCCTCCTCCTGCTGCTCTCGGG |
| | | AGGCCTGGCCCTGACCGAGACCTGGGCCTGTGAGTGCGGGG |
| | | TTGGGAGGGAAGCGGCCTCTGCGGAGAGGAGCGAGGGGCCC |
| | | TCCCGGCGAGGGCGCAGGACCCGGGGAGCCGCGCAGGGAG |
| | | GTGGGTCGGGCGGGTCTCAGCCCCTCCTCGCCCCCAGGCTCC |
| | | CACTCCATGAGGTATTTCGACACCGCCGTGTCCCGGCCCGGC |
| | | CGCGGAGAGCCCCGCTTCATCTCAGTGGGCTACGTGGACGA |
| | | CACGCAGTTCGTGCGGTTCGACAGCGACGCCGCGAGTCCGA |
| | | GAGGGGAGCCGCGGGCGCCGTGGGTGGAGCAGGAGGGGCC |
| | | GGAGTATTGGGACCGGGAGACACAGAAGTACAAGCGCCAG |
| | | GCACAGGCTGACCGAGTGAGCCTGCGGAACCTGCGCGGCTA |
| | | CTACAACCAGAGCGAGGACGGTGAGTGACCCCGGCCCGGGG |
| | | CGCAGGTCACGACCCCTCCCCATCCCCCACGGACGGCCCGG |
| | | GTCGCCCAGAGTCTCCCCGTCTGAGATCCACCCCAAGGTGGA |
| | | TCTGCGGAACCCGCCCAGACCCTCGACCGGAGAGAGCCCCA |
| | | GTCGCCTTTACCCGGTTTCATTTTCGGTTTAGGCCAAAATCCC |
| | | CGCGGGTTGGTCGGGGCGGGGCGGGGCTCGGGGGACTGGGC |
| | | TGACCGCGGGGGGGGGCCAGGGTCTCACACCCTCCAGAGG |
| | | ATGTCTGGCTGCGACCTGGGGCCCGACGGGCGCCTCCTCCGC |
| | | GGGTATGACCAGTCCGCCTACGACGGCAAGGATTACATCGC |
| | | CCTGAACGAGGACCTGCGCTCCTGGACCGCCGCGGACACCG |
| | | CGGCTCAGATCACCCAGCGCAAGTTGGAGGCGGCCCGTGCG |
| | | GCGGAGCAGCTGAGAGCCTACCTGGAGGGCACGTGCGTGGA |
| | | GTGGCTCCGCAGATACCTGGAGAACGGGAAGGAGACGCTGC |
| | | AGCGCGCAGGTACCAGGGGCAGTGGGGAGCCTTCCCCATCT |
| | | CCTATAGATCTCCCGGGATGGCCTCCCACGAGGAGGGGAGG |
| | | AAAATGGGATCAGCACTGGAATATCGCCCTCCCTTGAATGG |
| | | AGAATGGCATGAGTTTTCCTGAGTTTCCTCTGAGGGCCCCCT |
| | | CTGCTCTCTAGGACAATTAAGGGATGAAGTCTCTGAGGAAA |
| | | TGGAGGGGAAGACAGTCCCTGGAATACTGATCAGGGGTCTC |
| | | CTTTGACCACTTTGACCACTGCAGCAGCTGTGGTCAGGCTGC |
| | | TGACCTTTCTCTCAGGCCTTGTTCTCTGCCTCACACTCAATGT |
| | | GTCTGAAGGTTTGATTCCAGCTTTTCTGAGTCCTGCAGCCTC |
| | | CACTCAGGTCAGGACCAGAAGTCGCTGTTCCTCCCTCAGAGA |
| | | CTAGAACTTTCCAATGAATAGGAGATTATCCCAGGTGCCTGT |
| | | GTCCAGGCTGGCGTCTGGGTTCTGTGCCGCCTTCCCCACCCC |
| | | AGGTGTCCTGTCCATTCTCAGGATGGTCACATGGGCGCTGCT |
| | | GGAGTGTCCCAAGAGAGATGCAAAGTGTCTGAATTTTCTGA |
| | | CTCTTCCCGTCAGAACCCCCAAAGACACACGTGACCCACCAC |
| | | CCCCTCTCTGACCATGAGGCCACCCTGAGGTGCTGGGCCCTG |
| | | GGCTTCTACCCTGCGGAGATCACACTGACCTGGCAGCGGGA |
| | | TGGGGAGGACCAGACCCAGGACACCGAGCTTGTGGAGACCA |
| | | GGCCAGCAGGAGATGGAACCTTCCAGAAGTGGGCAGCTGTG |
| | | GTGGTGCCTTCTGGACAAGAGCAGAGATACACGTGCCATAT |
| | | GCAGCACGAGGGGCTGCAAGAGCCCCTCACCCTGAGCTGGG |
| | | GTAAGGAGGGGAATGGGGGGTCACATCTCTTATCAGAGAAA |
| | | GCAGAAGTCCTTCTGGAGCCCTTCAGCCGGGTCAGGGCTGA |
| | | GGCTTGGGGGTCAGGGCCCCTCACCTTCTCCTCCTTTCCCAG |
| | | AGCCATCTTCCCAGCCCACCATCCCCATCATGGGCATCGTTG |
| | | CTGGCCTGGCTGTCCTGGTTGTCCTAGCTGTCCTTGGAGCTG |
| | | TGGTCACCGCTATGATGTGTAGGAGGAAGAGCTCAGGTAGG |
| | | GAAGGGGTGAAGAGCGGGGTCTGGGTTTTCTTGTCCCACTG |
| | | GGAGTTTCAAGCCCCAGGTAGAAGTGTGCCCCGCCTTGTTAC |
| | | TGGAAGCACCATCCACACATGGGCCATCCCAGCCTGGGACC |
| | | CTGTGTGCCAGCACTTACTCTTTTGTGAAGCACATGTGACAA |
| | | TGAAGGACGGATGTATCACCTTGATGATTATGGTGTTGGGGT |
| | | CCTGATTCCAGCATTCATGAGTCAGGGGAAGGTCCCTGCTAA |
| | | GGACAGACCTTAGGAGGGCAGTTGGTCCAGAACCCACAACT |
| | | GCTTTCCCCATGTTTCCTGATCCTGCCCTGGGTCTGCAGTCGT |
| | | AGTTCTGGAAACTTCTCTTGGGTCCAAGACTAGGAGGTTCCC |
| | | CTAAGATCACATGGCCCTGCCTCCTCCCAGTCCCCTCATAGG |
| | | GCATTTTCTTCCCACAGGTGGAAAAGGAGGGAGCTGCTCTCA |
| | | GGCTGCGTGTAAGTGATGGCGGCGGGCGTGTGGAGGAGCTC |
| | | ACCTACTCCATAATTCCTCTTGTCCCACATCTCCTGCGGGCTC |
| | | TGACCAGGTCTTTTTTTTTGTTCTACCCCAGGCAGCAACAGT |
| | | GCCCAGGGCTCTGATGAGTCTCTCATCACTTGTAAAGGTGAG |
| | | ATTCTGGGGAGCTGAAGTGGTCGGGGGTGGGGCAGAGGGAA |
| | | AAGGCCTGGGTAATGGGGATTCTTTGATTGGGACGTTTCGAG |
| | | TGTGTGGTGGGCCGTTCAGAGTGTCATCGCTTACCATGACTG |
| | | ACCTGAATTTGTTCATGACTATTGTGTTCTGTAGCCTGAGAC |
| | | AGCTGCCTGTGTGGGACTGAGATGCAGGATTTCTTCACACCT |
| | | CTCCTTTGTGACTTCAAGAGCCTCTGGCATCTCTTTCTGCAAA |
| | | GGCACCTGAATGTGTCTGCGTCCCTGTTAGCATAATGTGAGG |
| | | AGGTGGAGAGACAGCCCACCCCCGTGTCCAC |
| 15 | HLA-C | TCAGGCACACAGTGTGACAAAGATGCTTGGTGTAGGAGAAG |
| | | AGGAATCAGGACGAAGTCCCAGGTCCCGGGCGGGGCTCTCA |
| | | GGGTCTCAGGCTCCAAGGGCCGTGTCTGCACTGGGGAGGCG |
| | | CCGCGTTGGGGATTCTCCACTCCCCTGAGTTTCACTTCTTCTC |
| | | CCAACCTGCGTCGGGTCCTTCTTCCTGAATACTCATGACGCG |
| | | TCCCCAATTCCCACTCCCATTGGGTGTCGGGTTCTAGAGAAG |
| | | CCAATCAGCGTCTCCGCAGTCCCGGTTCTAAAGTCCCCAGTC |
| | | ACCCACCCGGACTCAGATTCTCCCCAGACGCCGAGATGCGG |
| | | GTCATGGCGCCCCGAACCCTCATCCTGCTGCTCTCGGGAGCC |
| | | CTGGCCCTGACCGAGACCTGGGCCGGTGAGTGCGGGGTTGG |
| | | GAGGGAAACGGCCTCTGGGGAGAGGAGCGAGGGGCCCGCC |
| | | CGGCGAGGGCGCAGGACCCGGGGAGCCGCGCAGGGAGGAG |
| | | GGTCGGGCGGGTCTCAGCCACTCCTCGTCCCCAGGCTCCCAC |
| | | TCCATGAGGTATTTCTCCACATCCGTGTCCTGGCCCGGCCGC |
| | | GGGGAGCCCCGCTTCATCGCAGTGGGCTACGTGGACGACAC |
| | | GCAGTTCGTGCGGTTCGACAGCGACGCCGCGAGTCCAAGAG |
| | | GGGAGCCGCGGGAGCCGTGGGTGGAGCAGGAGGGGCCGGA |
| | | GTATTGGGACCGGGAGACACAGAAGTACAAGCGCCAGGCAC |
| | | AGGCTGACCGAGTGAACCTGCGGAAACTGCGCGGCTACTAC |
| | | AACCAGAGCGAGGACGGTGAGTGACCCCGGCCCGGGGCGCA |
| | | GGTCACGACCCCTCCCCATCCCCCACGGACGGCCCGGGTCGC |
| | | CCCGAGTCTCCCCGTCTGAGATCCACCCCGAGGCTGCGGAAC |
| | | CCGCCCAGACCCTCGACCGGAGAGAGCCCCAGTCACCTTTA |
| | | CCCGGTTTCATTTTCAGTTTAGGCCAAAATCCCCGCGGGTTG |
| | | GTCGGGACTGGGGCGGGGCTCGGGGGACCGGGCTGACCACG |
| | | GGGGCGGGGCCAGGGTCTCACACCCTCCAGAGGATGTTTGG |
| | | CTGCGACCTGGGGCCGGACGGGCGCCTCCTCCGCGGGTATA |
| | | ACCAGTTCGCCTACGACGGCAAGGATTACATCGCCCTGAAC |
| | | GAGGATCTGCGCTCCTGGACCGCCGCGGACACGGCGGCTCA |
| | | GATCACCCAGCGCAAGTGGGAGGCGGCCCGTGAGGCGGAGC |
| | | AGCGGAGAGCCTACCTGGAGGGCACGTGCGTGGAGTGGCTC |
| | | CGCAGATACCTGGAGAACGGGAAGGAGACGCTGCAGCGCGC |
| | | GGGTACCAGGGGCAGTGGGGAGCCTTCCCCATCTCCCGTAG |
| | | ATCTCCCGGGATGGCCTCCCACGAGGAGGGGAGGAAAATGG |
| | | GATCAGCGCTAGAATATCGCCCTCCCTTGAATGGAGAATGG |
| | | GATGAGTTTTCCTGAGTTTCCTCTGAGGGCCCCCTCTGCTCTC |
| | | TAGGACAATTAAGGGATGAAGTCCTTGAGGAAATGGAGGGG |
| | | AAGACAGTCCCTGGAATACTGATCAGGGGTCCCCTTTGACCA |
| | | CTTTGACCACTGCAGCAGCTGTGGTCAGGCTGCTGACCTTTC |
| | | TCTCAGGCCTTGTTCTCTGCCTGACGCTCAATGTGTTTGAAG |
| | | GTTTGATTCCAGCTTTTCTGAGTCCTTCGGCCTCCACTCAGGT |
| | | CAGGACCAGAAGTCGCTGTTCCTCCCTCAGAGACTAGAACTT |
| | | TCCAATGAATAGGAGATTATCCCAGGTGCCTGTGTCCAGGCT |
| | | GGCGTCTGGGTTCTGTGCCCCCTTCCCCACCCCAGGTGTCCT |
| | | GTCCATTCTCAGGATGGTCACATGGGCGCTGTTGGAGTGTCG |
| | | CAAGAGAGATACAAAGTGTCTGAATTTTCTGACTCTTCCCAT |
| | | CAGAACACCCAAAGACACACGTGACCCACCATCCCGTCTCT |
| | | GACCATGAGGCCACCCTGAGGTGCTGGGCCCTGGGCTTCTAC |
| | | CCTGCGGAGATCACACTGACCTGGCAGTGGGATGGGGAGGA |
| | | CCAAACTCAGGACACCGAGCTTGTGGAGACCAGGCCAGCAG |
| | | GAGATGGAACCTTCCAGAAGTGGGCAGCTGTGGTGGTGCCT |
| | | TCTGGAGAAGAGCAGAGATACACGTGCCATGTTCAGCACGA |
| | | GGGGCTGCCGGAGCCCCTCACCCTGAGATGGAGTAAGGAGG |
| | | GGGATGAGGGGTGATGTGTCTTCTCAGGGAAAGCAGAAGTC |
| | | CTGGAGCCCTTCAGCCGGGTCAGGGCTGAGGCTTGGAGGTC |
| | | AGGGCCCCTCACCTTCCCCTCCTTTCCCAGAGCCGTCTTCCC |
| | | AGCCCACCATCCCCATCGTGGGCATCGTTGCTGGCCTGGCTG |
| | | TCCTGGCTGTCCTAGCTGTCCTAGGAGCTATGGTGGCTGTTG |
| | | TGATGTGTAGGAGGAAGAGCTCAGGTAGGGAAGGGGTGAG |
| | | GAGTGGGGTCTGGGTTTTCTTGTTCCACTGGGAGTTTCAAGC |
| | | CCCAGGTAGAAGTGTGCCCCACCTCGTTACTGGAAGCACCAT |
| | | CCACACATGGGCCATCCCAGCCTGGGACCCTGTGTGCCAGC |
| | | ACTTACTCTGTTGTGAAGCACATGACAATGAAGGACAGATG |
| | | TATCACCTTGATGATTATGGTGTTGGGGTCCTTGATTCCAGC |
| | | ATTCATGAGTCAGGGGAAGGTCCCTGCTAAGGACAGACCTT |
| | | AGGAGGGCAGTTGCTTCAGAACCCACAGCTGCTTTCCCCGTG |
| | | TTTCCTGATCCTGCCCTGGGTCTGCAGTCATAGTTCTGGAAA |
| | | CTTCTCTTGGGTCCAAGACTAGGAGGTTCCCCTAAGATCGCA |
| | | TGGCCCTGCCTCCTCCCTGTCCCCTCACAGGGCATTTTCTTCC |
| | | CACAGGTGGAAAAGGAGGGAGCTGCTCTCAGGCTGCGTGTA |
| | | AGTGATGGCGGTGGGCGTGTGGAGGAGCTCACCCACCCCAT |
| | | AATTCCTCTTGTCCCACATCTCCTGCGGGCTCTGACCAGGTC |
| | | TTTTTTTTTGTTCTACCCCAGCCAGCAACAGTGCCCAGGGCT |
| | | CTGATGAGTCTCTCATCGCTTGTAAAGGTGAGATTCTGGGGA |
| | | GCTGAAGTGGTCGGGGGTGGGGCAGAGGGAAAAGGCCTAG |
| | | GTAATGGGGATCCTTTGATTGGGACGTTTCGAATGTGTGGTG |
| | | AGCTGTTCAGAGTGTCATCACTTACCATGACTGACCTGAATT |
| | | TGTTCATGACTATTGTGTTCTGTAGCCTGAGACAGCTGCCTG |
| | | TGTGGGACTGAGATGCAGGATTTCTTCACACCTCTCCTTTGT |
| | | GACTTCAAGAGCCTCTGGCATCTCTTTCTGCAAAGGCATCTG |
| | | AATGTGTCTGCGTTCCTGTTAGCATAATGTGAGGAGGTGGAG |
| | | AGACAGCCCACCCCGGTGTCCACCGTGACCCCTGTCCCCACA |
| | | CTGACCTGTGTTCCCTCCC |
| 16 | Beta-2M | TTTCAGTGGCTGCTACTCGGCGCTTCAGTCGCGGTCGCTTCA |
| | | GTCGTCAGCATGGCTCGCTCGGTGACCCTGGTCTTTCTGGTG |
| | | CTTGTCTCACTGACCGGCCTGTATGCTATCCAGAGTGAGTGC |
| | | CTCTTTCCCCTCTCTTGGCATTAAATTTTTAGTTCTCCTTAGTT |
| | | CTCCTTCCCGACGGGATTGGCCAGGGCTGCGTCTCTCGGGAA |
| | | AGGAGTGGGCGTCCCGCTGCTTACAGCTTTGAGGCTACAGG |
| | | GTGGATGCGCCTGTTTGCGAGTGTGCTGACGGTCAGCGCTGA |
| | | AGATGGGGAAGGCTTCTCTTTTTCTCCTCTGCTGGCGGGAGC |
| | | CCCTAGACTCCCTGAGCGCAAAAGGAGGGTGGGGCTGCCTG |
| | | GAAAGTGCAGGGTGGAGGGCTATGGGCACGCGGAAGCTGA |
| | | GCCCCGACCACGGGAAGTCAGACCGCCGCTGCGCAGCTTCA |
| | | TTTGCACTCGCAGAGAGGCTCGATCCAACCGAAGTCGAGAG |
| | | AAAATGGGCTTGGGTCCCAGACTCTGCGATGTTTCCAAAGCT |
| | | TTCTGATCATTAACTTTGTATCTAGTAATAACCTTAAAGGTC |
| | | GCCGGGCAGTGGTGGCTCACGCCTTTCATGCCAGCACTTGGG |
| | | AGGCAGAGGTAGGCGGATTTCTGAATTCGAGGCCAGCCTGG |
| | | TCTACAGAGTGAGTTCCAGGACAGCCAGGGTTACACAAAGA |
| | | AACCCTGTCTCGAAAAAATAAATAAAGGTCTCTGGGTATGA |
| | | GGCTTATTGCAATGCTGAGGACCTTGTGAGCTTCTTAAGTTT |
| | | GAAAGTACGTCTACAATGTAAATGTTATGGTTCCCCGTGTAT |
| | | TAAGACAGGGAACTAGAGGAGGCATCGAGATTTAAAGTAAC |
| | | TTATTCACACGATAGACTAATGGCAGTCGGAGCTGGAATTG |
| | | AGGCCGATCAATGCCCAGGCCTTTGGACTGTAACCGCCATTT |
| | | GTTTCTAGAAGTGTCTAGGCCTTCCTGGGTCATGGTCTGTGA |
| | | AGCAGTCTGAGATGCCTTGGACCCTTGGTACCTCACACAGCG |
| | | CTTCCTTTTTGGCACACTGCCTGGTTCTTTCCGCGATAGAGCC |
| | | TCTGCTTTCAGTTTTGAGACAACTAAGACTTTGCCTTAAGTC |
| | | AAGGTGGTTATGAACTCTGGAGTGAGGATTATTTCAGTGTGG |
| | | TGACCAAGACTCGTGAGGATAACTAGTAACTACCAAAGTCG |
| | | TGGAGGTAGAAATATGGCAGAGACCAGATTTAAACCTGACC |
| | | CAACACTGGACTTAATTGTGGAAGGGATGGTTCTTGTCCAGA |
| | | GAATTCCAGGCATGATGAACCTGTGGCATTTAGCATGTTCTT |
| | | GTTTCCAAAACCTCAAATGCAAGGAAAAATGTGGTTTATGCT |
| | | AATTTAAATTTATGGGGACAAAAAGAATTCAAAGCTTTACTG |
| | | AGACCTTCATTCTAGCTGGCCACAGTTTTCAGGTTTTCCTCTC |
| | | CCTACTGCCTTCCATGGGTGAATAAGAGGCTGCTCTTCAGAG |
| | | ACCGTGCACACTGAATAGATGTGTATCTGGGAGTATATTATG |
| | | GCTGGGCATAGTTGTGCACATCTATAATCTCAGTATTTAGGA |
| | | GACTGAGGCAGGTGGATCTCTGAATTTGAGACCAACCTGAT |
| | | CTACAAAGTGCATCCAGGACAGAAAAACGCTGTCTTGAAAA |
| | | ACCAAAGAAAGAAAGAAAGAAAGAAAAGAAAGTGTTTTAT |
| | | AAGCCAAGTGTGTTGAGTCATGCTGTGACCTCAGAGCTGGG |
| | | GAGGCTGAGACAGGCGTTCAAGGCCAGCCTGGAAGACAGTC |
| | | AGACTGTATCTCAAATTAAATAATAAGCATATAAGTAAATG |
| | | AAGGGGAAAACACAAAAAGAGCCAGTGAAATGGCTCAGCA |
| | | GGTACAGGCGCTTACCACTAAGCCTGAGTGTATCTCAGATCC |
| | | CACTTGGTGGACGGAGAGAACTGACTTTTAAATCCACACAT |
| | | GAGCTACGTTTCGTGCATGTGCACACACACACACACACACA |
| | | CACACACACACACACACACATATATACTCTCTCAGTAAAAC |
| | | AATAATTTTTTTAAGAAAGTAGATATATACATTAGTGATACC |
| | | TTGGTATCAGATCTGCATATTCTACACAGTAGTACTCCTTGT |
| | | CATGTTGGTTGAGAAGCAGAAACAGAGTTCAAAGATGGCAG |
| | | TGGTATGGCTCAGCAGCTAAAAGTACCTGCCATGCAACCTG |
| | | ACCACCTGAGTTCATAGAACCCACGACAGAAGGAGGAGAAA |
| | | ACTGTCTCCTGAAAGCCATCCAATGTCTTCACAAGTACACCT |
| | | TTGGGAACTTGACTGTGTGTATGTGTGTGTGCACATACAAAT |
| | | AACAATGGAAAGAGTTTACATTATTTTCTTTCCATGTCCCTA |
| | | TAGCGTGCAGTAGCTATTCCCAACATGTTGAGCGACTTCAGC |
| | | TATCTGAAAGCCCCATATCCCCTTGCACACACTGCTTACTTC |
| | | CAGCAGAGTGCATTCTCAATTGTCATGGTCCTCACATCTCCC |
| | | TCTGGTCCTTTACCCATCCCAACTCAGTTGCTTAAAGCTTTCC |
| | | TAGCTTCTGTGGTATTTTAAATTTTGTTTGTTTTCTAGTTCAT |
| | | GAAACCAGAGGTTTGGTTTCAGAATGCAAACTCTGGGCTGG |
| | | AACAGAGAAACCCTGGGTTGACTCGCATAGGAACCTCATTA |
| | | GGGAGGAGCCAATGCTAACACCTGCCACCTGAGGGGTAATT |
| | | GCTCAGCTCTCAGCACTGGATCAGACATATGTGTTGGGAAGT |
| | | CTAGGGAGGAGCAATAAGCAAAAACGAAAGGGGATGAAAA |
| | | TAAGAAAGAAAAGTGAGAGGGGCTGACACAGCGCACAAGC |
| | | ACTGCGGGCTTTGATGTGGATACTGTGAAGGGTGTGCAGAA |
| | | TGGGATGTGACGTTTGGAAAGTTGGTGGGATTTATTAAGGG |
| | | AAGGGAGGGGCACAGTTCTTGAGAGCTTCCAATAATAAAAG |
| | | CTCCTTAAAAATCCACTGACAGAAGACACTGCTAAAAGCCA |
| | | GGTAGGGAAAACAGAAGGTACTCGTAGGATTTTGAGGAAAA |
| | | GATACTACGTTTTCAAAATGTGGGTAGACTTTGGGGGAAGC |
| | | AGATCACTTATCCAGAGTAGAAATGGAACAGGGAGAAATAG |
| | | AGGAACAAATGTAAGATGGTGCACGGTGCAGACTGAGCTCT |
| | | GTTTTCATCTGTCTTCCCCTGTGGCCCTCAGAAACCCCTCAA |
| | | ATTCAAGTATACTCACGCCACCCACCGGAGAATGGGAAGCC |
| | | GAACATACTGAACTGCTACGTAACACAGTTCCACCCGCCTCA |
| | | CATTGAAATCCAAATGCTGAAGAACGGGAAAAAAATTCCTA |
| | | AAGTAGAGATGTCAGATATGTCCTTCAGCAAGGACTGGTCTT |
| | | TCTATATCCTGGCTCACACTGAATTCACCCCCACTGAGACTG |
| | | ATACATACGCCTGCAGAGTTAAGCATGCCAGTATGGCCGAG |
| | | CCCAAGACCGTCTACTGGGGTAAGCCTCAAGTTCTTCCTTAC |
| | | TTTCTGGACGCTCCATCTGTGTGGACTTAAAACTGCTTTGCT |
| | | ATTTTAAAAACCTGCATACTGAGATTGTTAGAGAATACCCCC |
| | | AAAAGACTGATCTGTGGTGCCAGCAGAGACTTACACAGCAG |
| | | GCCAGGAACCAGCAGTTCCCTACCCACTGTCCCAGACTGCTT |
| | | CCCTCTTTCCTGAGCTTCCTCCAGCTTTCCTGGCAGCCTGGTG |
| | | CATACTAAGTGTCAAAGGGCATGCCAGGTAGGACAGGCCTT |
| | | GGTCTGTAACCCCTGCCTTCAGGTACCTACAATTCTGGACTG |
| | | TTCCAGGGCCTTGGCAAGAAGCTAGTAGACAGGAGTGGATC |
| | | TCTGGAAAGTTCTCAACAAGTACTCAAGGCTAGCAACAGCA |
| | | ACCTAGTCTATCTGCCTTACTGCTTACAACCCCTGGATGTCT |
| | | GTTAATATGTCGTAGTATAAAAGTAAAGTTTTGTCTCCATTT |
| | | TCTTTTTTCATAGATCGAGACATGTGATCAAGCATCATGATG |
| | | GTAGGTTTCTTGACTTTAAATAATGGGCCTTTGGGGGTGGGG |
| | | TGGAGCTGGCGGTAGAGCACATGCCTAGCGTGCATAAGGCC |
| | | TTGAGTCCTGGTCCCAGCACTGCAGGAAGAGAATTGTTTGAT |
| | | TCACTATCCTAGGAGCTGTTTATAGACAGCTCAAACATGATA |
| | | AGCATCACTGTATGAAAGACATTAATTAGGGTGGCTTGATCA |
| | | GCTACAGAAGGATCCTTTGGGGTCACATTCTTTTATCCTGTG |
| | | GACTGGCAGGAAGGAGGAACGTAGCCATGTCACTGGCCCTC |
| | | TAAAGGGAGAACAGCACCTTTGTGCATGCAGCAAGGCAGGT |
| | | AGGCAGGTAGGCAGGTAGGCAGGCGGGTGGTCAGTTACACA |
| | | GCCTGCATGGGTTTGCCGACCTTCTGTTTTAACCCTGCCAGTT |
| | | CTGCTGAGAAGGCCTGGGACGATGAGCTTGCTGTCTTTGTAC |
| | | AGCAGTGGCCTTGCTCCTCACCCAGAGGAGCCGAGTGACAG |
| | | AGCTCTGCGGGTACATCTTAGCCCTTTCAGTCTCTAGTTGGT |
| | | AGGCACTAGATTTACCTTCTGGAGGCTTCCGGACACTCAGGG |
| | | AAAGAAATGGACAGGGTTGTAACTTCATGTAAGGCACCGTC |
| | | ACTGATGTGTCAGAAGGAAGTTGAGGAGCGTGAGAGGGAAC |
| | | GTGGGTGTCTCTGTCAGGTGGAGTCTAGTGGTAGAAAATCCA |
| | | GCTTTTCGGTGAAATCCAGGGCCCTTGAGGCCAAAAGCTCAC |
| | | TCAAATCTTGTATTATATTTATTTCTAGGAAAGGGGAATTGA |
| | | TGAAGGGGGTGGGGATGGGTGATCTGCCCAGACAAGCAGTT |
| | | ACCAAATGGTGGAGCCAGAACTCACCTCCTTTAAGAAGGTG |
| | | ATTGTCCTACAAACTCATTTGATGGTGAGGTCTGGAATGTAA |
| | | ATAATGTAATGTTCGGCTAACCTTCTCCTTCTCTCCCTCCCTT |
| | | CCCCTTTTCTTTTCAGCTCTGAAGATTCATTTGAACCTGCTTA |
| | | ATTACAAATCCAGTTTCTAATATGCTATACAATTTATGCACG |
| | | CAGAAAGAAATAGCAATGTACACATCACCTTCTTTATATCTT |
| | | ACTTTAAATATTTTATGCATGTTTTCAAAAAATTGGAAATAT |
| | | CCTAGATAGCTGAGCAATAAATCTTCAATAAGTATTTTGATC |
| | | AGAATAATAAATATAATTTTAAGAACAATAGTTGATCATATG |
| | | CCAAACCCTCTGTACTTCTCATTACTTGGATGCAGTTACTCAT |
| | | CTTTGGTCTATCACAACATAAGTGACATACTTTCCTTTTGGTA |
| | | AAGCAAAGAGGCCTAATTGAAGTCTGTCACTGTGCCCAATG |
| | | CTTAGCAATTCTCACCCCCAACCCTGTGGCTACTTCTGCTTTT |
| | | GTTACTTTTACTAAAAATAAAAAAC |
| 17 | HLA-DP | TCCAGCCTAAGGTGAACGTTTCCCCCTCCAAGAAGGGGCCCC |
| | | TGCAGCACCACAACCTGCTTGTCTGCCACGTGACAGATTTCT |
| | | ACCCAGGCAGCATTCAAGTCCGATGGTTCCTGAATGGACAG |
| | | GAGGAAACAGCTGGGGTCGTGTCCACCAACCTGATCCGTAA |
| | | TGGAGACTGGACCTTCCAGATCCTGGTGATGCTGGAAATGA |
| | | CCCCCCAGCAGGGAGACGTCTACATCTGCCAAGTGGAGCAC |
| | | ACCAGCCTGGACAGTCCTGTCACCGTGGAGTGGAGTGAGTC |
| | | TCTGATGACCRTCTAGACCCCACCTCTGAAGAGCAGGGGACT |
| | | CTCTGGCTCTGGGGTCCACTCATCTTATCTTCTGCATCTATAC |
| | | CCTGGGGCCATGTCCAAACCCCATCTTTCTTCTATACCAGCT |
| | | CCTGAGCATAGTTTGAAGCCAGGGCAATGGAGACTTCCTGA |
| | | CCTTGGCTTAGGGGTTCCTGAAGATTCATAGTTCTCCCCCTT |
| | | GTCAGAGAATCTAGGGACACTGGCTGATCTCGAAACCCTCA |
| | | CACACAGGAACTGACCTCACACATAGGAACAGTTCTCTTCTT |
| | | TCAGCATTTTAGCCTCTTCTCAGGCATTTTGAGAGGCAACTT |
| | | CTAGAATCAGCATTTGCCACCTTGTTGAGGTCACACCCCTGT |
| | | TCCGGACATGAGGGTGGCTCTTTCTGAATTTCCTCTTAGCAA |
| | | GCTTTTTCCCCTGCACTGTCTTCATCCCGATATTCTGCATCAC |
| | | GCTCCAGAATCTCAGACAGGACATGAGTAGGGATGCAGCTG |
| | | GTGGAGGTGACACTAAACCTGGGTCTGTCCTTCCCAGAGGC |
| | | ACAGTCTGATTCTGCCCGGAGTAAGACATTGACGGGAGCTG |
| | | GGGGCTTCGTGCTGGGGCTCATCATCTGTGGAGTGGGCATCT |
| | | TCATGCACAGGAGGAGCAAGAAAGGTGAGAAAGCCTGCAA |
| | | GGTGAGCGGGACTTACCTTCCCCTGGCATATTCACACTTACT |
| | | CCATGATGAGGGTTCAGACAGAAAAGAAATGTCAGAAAGCT |
| | | CTAGAGACCACTGAAATCAGATAGTCGGGGAACAAACATGA |
| | | CCTATGGCGAGAGGGGGATCCCAGGCTGGGATCTTAATGCA |
| | | GCCAGATGCATGAGGTCCCAGGTGCTCAGACTCCTGCGGGG |
| | | CATCCATTGAGTGGTGGTCAATGGAATTTGGTGGGATGGAA |
| | | ATGTTTCTCTAACTATCTGAGGTGGTTTCAATGGCTGAATAC |
| | | ATAACCTTTCCTCTTTCATTTCAGTTCAACGAGGATCTGCATA |
| | | A |
| 18 | HLA-DP | CTTTTCCAGGGACGGCAGGAATGCTACGCGTTTAATGGGAC |
| | | ACAGCGCTTCCTGGAGAGATACATCTACAACCGGGAGGAGT |
| | | TCGTGCGCTTCGACAGCGACGTGGGGGAGTTCCGGGCGGTG |
| | | ACGGAGCTGGGGCGGCCTGAGGCGGAGTACTGGAACAGCCA |
| | | GAAGGACATCCTGGAGGAGGAGCGGGCAGTGCCGGACAGG |
| | | ATGTGCAGACACAACTACGAGCTGGGCGGGCCCATGACCCT |
| | | GCAG |
| 19 | HLA- | GGTGGCTTCGTGGCCCATGTGGAAAGCACCTGTCTGTTGGAT |
| | DM | GATGCTGGGACTCCAAAGGATTTCACATACTGCATCTTCTTC |
| | | AACAAGGATCTGCTGACCTGCTGGGATCCAGAGGAGAATAA |
| | | GATGGCCCCTTGCGAATTTGGGGTGCTGAATAGCTTGGCGAA |
| | | TGTCCTCTCACAGCACCTCAACCAAAAAGACACCCTGATGCA |
| | | GCGCTTGCGCAATGGGCTTCAGAATTGTGCCACACACACCCA |
| | | GCCCTTCTGGGGATCACTGACCAACAGGACAC |
| 20 | HLA- | CTCCTACTCCAATGTGGCCAGATGACCTGCAAAACCACACAT |
| | DM | TCCTGCACACAGTGTACTGCCAGGATGGGAGTCCCAGTGTG |
| | | GGACTCTCTGAGGCCTACGACGAGGACCAGCTTTTCTTCTTC |
| | | GACTTTTCCCAGAACACTCGGGTGCCTCGCCTGCCCGAATTT |
| | | GCTGACTGGGCTCAGGAACAGGGAGATGCTCCTGCCATTTTA |
| | | TTTGACAAAGAGTTCTGCGAGTGGATGATCCAGCAAATAGG |
| | | GCCAAAACTTGATGGGAAAATCCCGGTGTCCAGAGGGTTTC |
| | | CTATCGCTGAAGTGTTCACGCTGAAGCCCCTGGAGTTTGGCA |
| | | AGCCCAACACTTTGGTCTGTTTTGTCAGTAATCTCTTCCCACC |
| | | CATGCTGACAGTGAACTGGCACGATCATTCCATCCCTGTGGA |
| | | AGGATTTGGGCCTACTTTTGTCTCAGCTGTCGATGGACTCAG |
| | | CTTCCAGGCCTTTTCTTACTTAAACTTCACACCAGAACCTTCT |
| | | GACATTTTCTCCTGCATTGTGACTCACGAAATTGACCGCTAC |
| | | ACAGCAATTGCCTATTGGG |
| 21 | HLA- | GGGCATTTTATACTTAGAGTTTTTTTTGGCTTAATAGCTACAG |
| | DOA | GTGGCTTGTGGCTACTGCACAGGACAGCACAGTCCTTGAGG |
| | | AAGGGGATCATGTCCTGCTCTTCTGTGCTCCCAGAGCAGCAC |
| | | ACAGTGCCTAAGTCACATCAGGTGTCCTATAAACATGTATTT |
| | | AATGAAGAATATCCCCCAACACCTACCTGAACTTGTCATGCT |
| | | CCAAAGAACATGACAGATACAACTTTACCTCAAAGTCAGCA |
| | | TTTTAGACAATTTAGAACAACTGGATTTGTGTCATTTTTAAA |
| | | GGACACTTTAGTAAGTTTTTGTCGAATCTGTGTTTGCCTCATT |
| | | ATTAGACTCTAGTCCCAATAACAGTACCTCTCCCTAGAGTCA |
| | | GGGTTATCCACTTTATTCACTTTTATTTGTTATCATTGACTTA |
| | | TTTATTTAATAACGTGCTAGCGTTTAGGGGGAAAAGGAAGA |
| | | TTGTGTAACAAAGATTCAATCATCCATGATCCCTTCTTACAG |
| | | GCCCTTTCCATCTAGGAGGGAATGTGGACCATACATATACAT |
| | | TCCAAGAAAACAAATAATGAGGAAGCCCTGGTGAGACTGAA |
| | | AGGTGTTCTGAACTGGGTTGCTTGCTGGCTGTGGCTATGAGA |
| | | AGGTTCCTGGAACCTTCTGCTCTTGCCTCCCCGGTGTTGGCCT |
| | | TGAGCAATCACTGCCTGCTCCCTGCTGCAAAAAACGATGGC |
| | | AAGATCCTGATTGCCTGTCCAGCTGTTGAGGGGTGGGTGAG |
| | | AAAATCCAAGCTGAAATCCTGCTGGAGTGGGAGGAGCTGGG |
| | | GACTGCCCTAGTCATTGCCTCCTCTGCCACCCCAGCTATCAG |
| | | TGCTCCCTACGCCTCCTAGTGCTCCCACCCTGAAGTCCTGAC |
| | | CTGAAGTGACACCCAAAGGGTAGTGACTATGGTTTTGCGCTG |
| | | GCCTAGGACTCCAGCTGGCCCTCCTTTCTCTTTACACACATG |
| | | ACACCAACACAGATGGCCACTAGTGGGTCAAAATAATACCT |
| | | TTAGGGAGTGCCGACACCTCAAAAGAAAAACAGCAAACAGG |
| | | ACCATCTATGTCCTTGAAAGAAGTGGTAGAAAATATAGACC |
| | | AGCCGCTTAAAATGAGCATTAAAAGTGCACTTTAAAAGTCA |
| | | GAAGCTATTATAATATGAAAAAATAGAGAATATAGGGAAAA |
| | | CACCTAGAAGTTCTAGGTGTAAAAAATATATATAATAGTCA |
| | | AAGTAAAAGCCACATTAATGGGATAAATAGTGGGAGAGATG |
| | | CAACTGAAAAACAAAACAGAAAGATGGAGGACTACACTGA |
| | | GGAGCTAGTTCAGAACAAAAGAGGGAAGAATTGAGAAGTC |
| | | ATCAATAGAAAAGTCAGGGGAAAAGGAGGAGAGAATTATC |
| | | CAAATATAGATTATATGAGTTTCACAAAGACAAAAAATAGA |
| | | GGAGAAAAATAGTTAAAGAAATAAAAAGATGAATTCCCAGA |
| | | ATTAAAAGATAGAGAAGATCCATAATGTCTTACAGAGAGAA |
| | | GGAAAAACCCACTCTAGACACACTAGCAAAATTTAAAAATA |
| | | TCAAAGACAATGAGAAAATTCTAGAAGATTCCAGCATGAAA |
| | | GACTGACATGGGATTTTCTAACTGTGACATTGGATGCAAAAA |
| | | AAAAAATTTGGAATGATGTTATTTAGAATATTAAAGGGGAA |
| | | AATACTTCAAATAGTATGTAATACACAGCTAAACTTTATTCA |
| | | AAAGTGCTGGTATAATTTTAAAAAATTATCAAGCATACAAG |
| | | ACTTTAGAAGGTTTGCTATGCAAATTCCCACATCTGAAAGTT |
| | | TTTGGAAGAAGAATTCTAATCAGAGAACGCATTCTAGAAGG |
| | | TGCCAAAAGATATGAAAAGTAATGTTGACCAAATAATTTGG |
| | | TGAAGGAGAAAATATGTGAATAAATTTGAAAGCATGCTAAA |
| | | GTTCTTATCCAGTCAGAGGGAAGATGGAGAAGGTATCGGTA |
| | | ACACTTAAAAAAAAAAAAAGGCAATGAAGATTAAAAAAGA |
| | | AAACTTGGCCGGGTGTGATGGCTCACGCCTGTAATCCTAGCA |
| | | GTTTGTGAGGCCGAGCCAGGCAGATTGCCTGAGCTCAGGAG |
| | | GATAAGCCTGGGCAACACGGTGAAACCCCGTCTCTACTAAA |
| | | ATACAAAAAAAAAAAAAAAATAGCCAAGCATGGTGGCATG |
| | | CACCTGTAGTCCCAGCCACTCGGGAAGCTGAGGCAGGAGAA |
| | | TCGCTTGAACCCGGGAAGCAGAGGTTGCAGTGAGCCAGCCC |
| | | AGATCATGCCACTACACTCCAGCCTGGCAACAGAGCGAGAC |
| | | TCCGTCTCAAAAACAAACAAACAAACAAACAAAAAAACTTT |
| | | AAAAAATAGAAATATTGAAATAAATAAGGTATTACAAACAA |
| | | GCCCAAGTGCATCAATATTCACAATAAACACAATCAGAATG |
| | | CAGCAAAGACAGTGCCATGTGCTCACCAAACCATTATATCTT |
| | | CCTGCACACATAGGTAACGTACATTTTTCCATCCCCGTGCGC |
| | | CTATGTGGGGTCATTGTTTACTTCTTGCCAATGGAATGTGAG |
| | | CAGAGATGGCAAATAGTTTCTGGTCCAAAACCATGGAAACA |
| | | ATGTGCCTTTCCCAGGTTCTCTCTCCTACCTGCAAAGCTAGG |
| | | AGTTCATGAAGTGCTTTTCAATAATAGAGATGCAAAGTGGA |
| | | GCTTAGCCACCTTGAGGCATGTTTTGGAAAAGAGCTACAGA |
| | | GTGCCCAACCTGCCTTGAACTACACGTGGCTAAAAAATAAA |
| | | CCTGTGTTTTGTTAAACCACTGAGGCTTCAGTGTTTGTCTCTT |
| | | GCAGAAACAAGTTTGCTCACTTTGACTACCATATGAATTGAA |
| | | CATAGGTTAGAAGACAGAGATCGTCAGATTGAATCAGAAGC |
| | | AAAAATCTATATTTATGACATTTCAAGGGCTACATCTAAAGC |
| | | ATAAGTACAAAGGAAGAGTAAAAACAAAAGAGTAGAAAAA |
| | | GATACAGTGAACAAATACCCAAAGACAGCTGTAGAACTTAT |
| | | TACTATCAGACAAGACAGAGTAGGACAGAAAGATTCTCAGG |
| | | ACAAATTGGGAAAAGGCTCAGTTCACCAAGACTGTAATAAT |
| | | TCCCAAGAGTAAACACCTTAATAACCTAAATGCAGAGAGGG |
| | | CAAAAGTTGATTTAAAAATTAGAAACAAGTTGAAAAACCTA |
| | | CAGTCACAGTTAGAGATTTAAATATACCATTCATAGGCCAGG |
| | | AATTGTGGGGCATGCCTATAGTCCCAGCTACTCTGGGAGGCT |
| | | GAGGCAGGAGGATCGCTTAAGCCCAGCTGTTTGAGGCTGCA |
| | | GCGAGCCATGATTACTTCACTGCCCTCCAGCCTGAGCAACAG |
| | | AGCAAGGCCCTGTCTCTAAAAAATAAATAAATAAATGCACC |
| | | ACTCTTAGTTATGTATAGGCCAGTCCAAAGATAAAGAAAAC |
| | | CGTAATAAATTAGAATGACAAAATCCACATGCTTGATCCAAT |
| | | AAACATGTACATGTATAAAATTCTGCATCACATTTATTAGAG |
| | | CCCATGTGAAATATTTACAGAAGTTCACCACACTCCAAGCCA |
| | | TAGAGGATTATCATGTAATTAAATTATAAGTTAATAACAAAA |
| | | AACATACTTTAAAAAATCTATGTATGTTTTCAAATAAAAAAA |
| | | ACACCCGATACTCGACTGTTATTAACCAAAGAAGCAACATTC |
| | | TTTATCATGAGTTTTTCATGGCTTTCCCGTCTTTCATCTCATG |
| | | GCTCCAGCTCCCACCTCACTTGGGGGGAGGTGAAGGTGGCA |
| | | AGGGTCCTAACCAGGGCAGGACCAAAGCAGTGAAGACACTG |
| | | TTCATTCATGCGCAGGTGTCTCCTAGGCGCCTATGATGTGCT |
| | | AGGCATTACGTAGACACTGTGGACTAGAAACGAAAAAACTA |
| | | AGATTAGGGCTCCCCATAAACCGGGCCATGCAAATCAATTA |
| | | CTAGCTGTCATTTGATTAATTTGAAGTCAGAAGCTGTGTCTG |
| | | CCCCACTTAAAATAGGCATCCCTTATTCATTGAGGAAAGAGC |
| | | ATTGGAGCAGGTTTTACCAGTCCACAGACTACCCGAGAACC |
| | | ACCTGGGGAGCATGCTGAAAACACAGGTTCCCTGGCCCTGC |
| | | CTTGGAAAGGCTAATCTAACCCATTAAGTAAAGAGGAAACT |
| | | CAGGAACACGTATTATAACGAACCCCCCAGGTGATTCTCATT |
| | | TGGAACCAGAGACCCGCATGATTTCCTAGCTCCCTCCAAGTT |
| | | GAAAATCATCGATTTCATGGTCTTTGACACATGGCCTAATTT |
| | | GGTTAGATTTCTCTTTACACAGACGTTAGCTTTACGTTGTTGT |
| | | TGTTTTGTTTTGTTTTGTTTTTTAAATATATATACTGGACTAA |
| | | ATTCTCCCAGTGCCTCTTATTAGCCGAGCCCAACCAGAAGCC |
| | | AAAGGCAAGGGTGATGCCGTCCTCCAGCCTGTGTCAGCCTG |
| | | GAGGAGGTGCCCGGGTCCTGGAGGGACCAACAGAGAGGAC |
| | | CAGCACCTCTGCCCTCCTCGGGGAGTCCACGGGAGCTCTTTA |
| | | CACTTTGCTTTAGACCAAACAACTCCAACAATTACACATTTT |
| | | TTCTTACACAATGAAATTAAATAATAATAAAAGAAAAATAA |
| | | AATAACATTTTTGAAAGTGATTTAAAGTTTTAAGAGGTTTTT |
| | | ATTCCATTGTGTAGCCAATGTTTTCCTTTTAAAGATTATTGGT |
| | | AAAGTTTATTTTACTTTCTAACTCAAAATTTGTCCACGGAAA |
| | | CTTCTTGGGAAAGTAGGATTCCCTGCATCGCAGATGGACATA |
| | | GAGAGAGGATTTGTTGGAGCTCAGCTGCGCGGTGCTCTGAC |
| | | GGCCTTTTCTCTCTCTTCAGGGGGTCCCTTCTTGGAGGACGT |
| | | GGTAATAGTTGGTCCAAGCCCCTCCCATCCTCCAAAACTTCC |
| | | TCTGGCCTCTCCTGGGGGCCCAGTGAATCCTTCCACCTTCTC |
| | | ACCCCGGCTCTTTCCCTCTTTACCCAGCAACAGATACATTCA |
| | | CTCAGAGAATTTCTGTGATTGGCTGAAGACAGCAGGGGTCG |
| | | CCCCCATCCTCGAATCTGTTTTCTTCTTCTTTACCTCCGCCTT |
| | | GTTCCTGTCCTCACCACACGGACTGAGACTGATTTGATTAAA |
| | | GCACCAGAGTGTAATGGCCCTCAGAGCAGGGCTGGTCCTGG |
| | | GGTTCCACACCCTGATGACCCTCCTGAGCCCGCAGGAGGCA |
| | | GGGGCCACCAAGGGTGAGTGCGAGGGCGAGGAGGGTGCGG |
| | | CGGGGAGCAGAGATTTACGGAGTTGGGTTACATGAGGAGGT |
| | | GGCATGGAGGATGCTTGTTCCTCTCGCTCTCTGGTTTATGGG |
| | | CAACTTCCTTCACCAAGAGACACCCAATCCCCCTCATCTCTG |
| | | TCACATCCACTCTGGACCTAAATGAAGATGCAGCTCGGTCAG |
| | | CTGCGCAGGTGCCCCAGTCAGCCTTTGCTGACGTTCAGATTT |
| | | CTCCTCATTCCTTCCTCCTTCCTGAGACCCAAACCTCCACCCA |
| | | ACAGATGCCAGCAAGCACCCTGATTTCTCTACCACCCCTGGC |
| | | CGGGAATGTGCCCGATCAAGTCCAGTTCTGTTGCAGTATTTA |
| | | TGCCCATGCCGGTAGTTAACTATTTACCTGTCTTTGTTCTTCG |
| | | GGAGACATGAGCTGGGGTGCGGGTCTACAGATGGTTCATCT |
| | | TTTTTTTCTTTTATTTCCCTGGCCCCCCATTGTGCTGGGTGCA |
| | | TGCTAGTTCCTCAATAACTGTTGCTCAAACAACTTCATAGAG |
| | | TTCTACAAGAATTAAAACTTAATCCCTAACTTCCAGAAAACT |
| | | AGACAACAGTTATGGAAGAGCCACACTCAGTCATAATGCTC |
| | | TGAGATGGAGGAATTGGGACATGAACCTTGACTTCTGACTCC |
| | | TCGTCCAGTGCTCTTTGTAATGCCTTGAGTTGCCTCTCCCTAT |
| | | CCCCTTGGTCTCTGGGTCACTAACCTTAATTCTTACCCCTGCC |
| | | AGCCGTGGCCTGTTTCCCCTCACACCCACCTGCACTGCATCT |
| | | CTGTGCAAAGCTCCATACTCTCCTGTCCTTAATCATTCCTCCT |
| | | CATCCCACCCCCACAGTCCCACCAGCTCACAACACAGGGCCT |
| | | GACAACACAGCCAGGGCAGATGACCACAGCCAAGATTTAAA |
| | | TTCTGGAACCCCAAGCATGATTTTAGGCAGTCCCTCCTCTTC |
| | | CCATCCTGATATGCCAGACTGCACTGTCTCTGTGCCGACTGC |
| | | AGTGTGCTGGGATGAGCCTCTTTCTTCCTGTTCTCTCTCCCTT |
| | | TCTCTCTCCCAGGGCCAGCCCAGTGTCAGAACAGGACTCTGT |
| | | CCCCACACAGAACCCAGGACGGGGCCCAGGCTCAGGGACTC |
| | | AACAATCACATATTGTGGATGAGACAGACACATTTTTTTCTC |
| | | TCTCCTTGACCCTGAACTCGCCAAACACAGCTGACCACATGG |
| | | GCTCCTACGGACCCGCCTTCTACCAGTCTTACGGCGCCTCGG |
| | | GCCAGTTCACCCATGAATTTGATGAGGAACAGCTGTTCTCTG |
| | | TGGACCTGAAGAAAAGCGAGGCCGTGTGGCGTCTGCCTGAG |
| | | TTTGGTGACTTTGCCCGCTTTGACCCGCAGGGCGGGCTGGCC |
| | | GGCATCGCCGCAATCAAAGCCCATCTGGACATCCTGGTGGA |
| | | GCGCTCCAACCGCAGCAGAGCCATCAACGGTACCGGCCCTC |
| | | CCTCTGCCCACCCAGTCAGGCGGGAAGGTCCAGAGAAACTT |
| | | CCTCCCAGTTCCTAGGCTCCCATCACTCTGGGGCGCGCTCTC |
| | | AGCGCCCGCGCCTGTCATGCCCTGTTCCTTTCTTTCCCAGGA |
| | | GGCTCCAGGTCTTCCCAGACCCCTTTGGCACCCCTCTCCTTG |
| | | AGGAATGACACCTCTCACCCGGACTCCCGCCCAGGGACCAG |
| | | TCAAATATAGGAGCTCCTGGCGTCCCCACTCCCTCCCCAGTC |
| | | TCCTCTCCCTCTGTTTTCCTCCTCTCCTGCCCCAGTGGATACC |
| | | CCAGAGCATCCCCTGCCCACAGATGGCTACAAAGGGGGAAC |
| | | GTCCCTTAATCCCAGTCCTAGTAAGGCCCTGGGGTGAGGGAT |
| | | GAGCCTGTGGACTCAGGGCCTGTTCCTCCTAGTGCCTCCACG |
| | | GGTGACCGTGCTCCCCAAGTCTCGGGTGGAGCTGGGCCAGC |
| | | CCAACATCCTCATCTGCATCGTGGACAACATCTTCCCCCCTG |
| | | TGATCAATATCACCTGGCTGCGCAACGGCCAAACTGTCACTG |
| | | AGGGAGTGGCCCAGACCAGCTTCTATTCCCAGCCTGACCATT |
| | | TGTTCCGCAAGTTCCACTACCTGCCCTTCGTGCCCTCAGCCG |
| | | AGGACGTCTATGACTGCCAGGTGGAGCACTGGGGCCTGGAT |
| | | GCGCCACTCCTCAGGCATTGGGGTACGGAGCCCCCTCCCCAT |
| | | GCACCCTCCTGGCCCCAGGTTTCCTTTACTCTAGAATCCTTTC |
| | | ATATACCACCAACTCCTTCCTTTCTCTCCTAGAGCTCCAGGT |
| | | GCCTATTCCACCACCAGATGCCATGGAGACCCTGGTCTGTGC |
| | | CCTGGGCCTGGCCATCGGCCTGGTGGGCTTCCTCGTGGGCAC |
| | | CGTCCTCATCATCATGGGCACATATGTGTCCAGTGTCCCCAG |
| | | GTGCAGAGGCCCCGGGAGTCTGGGGGGTGGGGGAGGAAAG |
| | | TGGATGACTCTGAACAGGACGTGGGTGGAGAATCAGAGATT |
| | | CTGTTGTGGGGAAAGAAGTCAGAAAAGAAATGGGCAGGGA |
| | | GAAAAGAAGCAGAGGTGGGGTGAGAGAGTGAGGTTTTGGG |
| | | GGAGGTGGGCACTCAGAGATAGGATCCCAGCATATTGAAAT |
| | | TGAGCAACCTCGATCGTATGTTTTCTGCTATTTTAGGTAATG |
| | | ATCCTTCTGAGAGAAATGACTTGTGGGAGACACCCTGCAGA |
| | | TCCTCATGGGTTTGTGACAGCCCCTGCGTGCTCAGTGCCCTT |
| | | TAAGTGCATCCCGCTGTGCTGACTTTGAGTGGGATCAACATC |
| | | TGTCCTACGGGTCCCCTCTTTTTTGGCCCCAGTATTCATGGCA |
| | | GGGTTTGTTGGACACCTACTAGCTTCCCTTCCCATTCAACAC |
| | | ACACACACATTCTTGCTCTACCCAAAGCTCTGGCTGGCAGCA |
| | | CTAAATGCTTTGGTGGTGTTTGCACTGTGTCCTTTCCAGGCCT |
| | | TGGCCAGTTCTTCCAGGGGTGAGGCATGTGGTGCTGGGGATT |
| | | GGCAGCCATCCTGGGGCCCACACAGGTGTGTCTTGCTCCATT |
| | | TGGCCCATTGTGTGTTACTTTGTGAATGAGCCATTTCACATG |
| | | GACTTCATGAAATTTGCCTCCTGAGTTCAGGTTTACCCTGAA |
| | | AGGGATGCAGATTATCCTGTTCCTCACGACCCCCTCAGCTAA |
| | | CAACAGTTCTGAAGGGTGCTGGGACAGGACAGGCTCATGGG |
| | | GACTCCACTCCTGCCTGGGTTTACTCTGTATGAAGAGGCCAC |
| | | TGGTATCCTGCCATGATGTTATCTCCTTTTTCTACTTTTCCCT |
| | | AGAGTCCCATGCATGATAAAGAGAGGCCCAAGGCTTGGATA |
| | | AGGTGGCCACTTCCCTCAGTGGAGTCAGTCATGTTAGGTAGG |
| | | AGGTGGTAGAGTCGGTCTGCGAGGTATCTCGTAAGAGGGGA |
| | | GGTCCACCTAGACACACTCTAAATATGTGGCCTAGAAGATTT |
| | | TGGTCTACTTTTCTGTGAACAGAATTTAAAACATACAAAGAG |
| | | ATAAATCACCATACCACATAGTTTATGTCAGGACCAAAATG |
| | | AGCAATACAGATTACGGTTTTCAAACCAGAATGCACATAAG |
| | | AACTGCTTGGGATCCTTTTAAAAGTACAGGCATTGGCCTGGT |
| | | GCAGTGGCTCATTCCTGTAATCCCAGCACTTTGGGAGGCCAA |
| | | GGGGACAGGACTGCTTGAGGCCAAGAGGTGGAAACCATCTT |
| | | GGGCTACATAGAGAGACCCCATCTCTACAAAGAAAGATTTA |
| | | AAAATTAACCAGGCATGGTGGCTCGCACCTGTATTCCCAGCC |
| | | ACTGGGGAGGCTGAGGCCGGAGGAGTGCTTGAGCCCAGGAG |
| | | TTCAAGGCTGCAGTGAGCCAAGATTGCGCCACTGCACTCCA |
| | | GCCTAGGTGACAGAGTGAGACCCTGTCTCTAAATAAATAAA |
| | | TAAATAAAATATAAAAATAACAGTCATCACCCAGACCTACT |
| | | GAATTAGAATCTCGGGAGTGCAGGGGGCAGCAACAGGGAG |
| | | GCTGTCTTTTCTGAGATGGGGTCTCACTCTGTCACCAGGCTG |
| | | GAGTGCCATGGCATGATCTCAGCTCACTGCAACCTCCACCTC |
| | | CTGAGTTCAAGCCATTCTCCTGCCTCAGCCTCCTGAGTAGCT |
| | | GGGACTACAGGTGTGCGCCACTACACTCAGCTAATTTTTGTA |
| | | TTTTAAGTAGAGACGGGGTTTCATCATGTTGGCCAGGATGGC |
| | | CTCCATCTCTTGACCTCGTGATCCACCCACCTTCCCTCCCAAA |
| | | GTACTGGAATTACAGGCATTAGCCACTGTGCCCAGCCGAGG |
| | | CTGTCATTTTTAACCGGCTCTGGATGACTCTGATGCAGCCAT |
| | | CCTGGACCTTGGCTGTGGTCTGGTAACTGGAACCCAGTGACG |
| | | TAATCAGGTGCCATCGGGGGTCATGGGAAAGGGGGATCCCC |
| | | AAGGTCTGAGGTGGACTAGGAAGGCTTTCTGAAGAACCTGG |
| | | GTCTGTTAGGGCATCAGCCAATCAAGGTACAAGTAAATAGA |
| | | GGCAAAATGAGGGTTTGAACTGTGAGCAGTTGGTCCTGGAA |
| | | AAGAAAGAAACCAAGAGATTATGGGGACTCAATGGGCTTCT |
| | | TAAGAGAGAATAAGTTGAAATCAATGACCAGAAGACCCTGA |
| | | TGGAAGTGGAGGAGAATCATCTCAGGCAAACTTTTTGTGTGC |
| | | CAGTAACAGAAACCCTCTTTGTGTGATCACATGCAAAGTATA |
| | | GGATATTTGCAATATAGCCATGGGGAGGAGTGCAGGGCCCA |
| | | AGGGTAGATTTTAGCCAGGCCTCCCAGGAACAGAACTCGGA |
| | | TCCGAAAAGCCCAGAGAAGCTAGAGCTGCCCCTCCAACACT |
| | | CTCGGATCCACATGGTCTGTGTTCTCTAGACCCCCCTGCATG |
| | | TTAGCGGTGTTCTCTCTCTGTGGACTGACTGTCCTTCTCAGTG |
| | | AACATGTCCACCCGACAGCTCCTGAGTTTATATCATCTCAAC |
| | | CCTCACAACCCACAGAGGCTGTGTCTCCTAGTCACAGCTTTA |
| | | AATTACTGGAAAAATAAATGACTGGCCAAACTTGGAGCAGG |
| | | TGTCCATCCCAGCCCTGTGTAGTTAGAGCAGGAATCAAGATC |
| | | TCAACACAAATGTGGCTGCCAAGCACTCAGCCCCGGGGCGA |
| | | GGGGTCAAGTTCTTCTCAGAGAAAGAGGAATAAGTTGGTTC |
| | | TCAGAAGACATCACAAGATACGTGTGTACCCAACAATCTCT |
| | | GATCTCTGCTGATCTTTTGCTTAGACGTTAACTTGATGCATCA |
| | | TTGGAAAGGTGTTTCTCTCATCTCTGTCCTAAGGCTTGATAA |
| | | AGTCATTAAAATTGTGTTCTTTTGACTAAAGAAATATGCTTT |
| | | TTTTTTACTGTTGCATATACTACCCTGAAGTCACTGGAACTTC |
| | | TAGGAGTAATTCCAGAGCTTTTAGATTTATGCACCTGCGTGT |
| | | ATACTCACATTTGTTTCTAGTCTCAAGGTACGTAGTCTTTTAT |
| | | TTTAAAAAACAAGTTGTCCTCCTGCATTCTTGATCTTTCACTC |
| | | TCTTTTAGAACAGACATCCAGTAGTCTCCTGTCCTTTGTATCC |
| | | AAACTCTCCCTTTTTGCAGGGTCACCCTACAAATCATAAGCA |
| | | TGTCATCTCTCCTATATAACAAGAACATCAAATCCTCTGTCT |
| | | GTCCTTGACCCAGTCTCCCTTCCACCCAAGCTCCTGGTCACG |
| | | CTCTCTGAGAGTGTCTACGTGGACTGCCTCCAGATCCCCTCT |
| | | TCCCATCCACTCTCTTTCGGTTTTAATTTTTAACCAAATTATG |
| | | TTATAGTTTAAAGAGTCAAATATTTCTACAATATTTGCTACA |
| | | GAAATACCAGTTTCCAGCCCCATCTCCCACCATATCCTCACC |
| | | CTTACAGAAAATAACTTTCAATTGTTCTAATATGTTTTGTTGG |
| | | TATTAACCTCCCATCTCTAAATAACATGTTTGTGTTGCTACAT |
| | | CTAGATTTTTCAGCTTTAGGCTTTATCTCTTTACCTCCTGCTG |
| | | TGGAAGAGGGGGATTTAGATTTTTTTCATCCTCAAAGAACAT |
| | | CATGCCCCCTTTCCCATGCCCTTTCTTTTAATGTGACTATATT |
| | | GTAATTTTTACAATATAGTAAAAGTTACTGTGTTTACTATGTT |
| | | TCTATCAATATGACCATGTAATGGCAAACCATAGAGCAAGC |
| | | CATGCTCACTCTTCCTTTTCTAGACGACTTCGTTTTCTCTGGA |
| | | GTATATAGTTGTCCTGTTTTCCTTTGTGTGGCTGTATTTGTAC |
| | | TTATTATTAATGAATCACAAAATTCCTCACAGTACAATCAAA |
| | | CACATCAGGTATTCTGTCAATTTTATCACCAGATATAACTCT |
| | | CCCAGAACCTTCTGTCTGCTCCATTCTTAACTCCTTGCCCTTG |
| | | ATGATACAGCGGTCACTCTGAGATCACTCTTCGCCATCCTCG |
| | | TCAGGATTTACCTATCCTCCGAGTTGGATCTTCTGCTTCTTGT |
| | | ATCCAGTCATTGCCCTTTTGTGGTCTGCCTTATTTTAATGAGG |
| | | CACCTCTTCTTTCCACTTCCTGAGAAACAGTGCATATGAGGA |
| | | AAACTTTTTATGCTTCGTATGTCGGAAAATATATTTATTCTAC |
| | | TCTCACACTTGAATAATAGCTTCCACGGTTATAAAATTCAAG |
| | | ATTGGAAATCATTTCCATCAGAATTTTTAAGGCATTTCTCTAT |
| | | AATCCCTTAGCTTCCTGCGTTATTGTTGATAATCCAAAAGAT |
| | | ATTGTGTCATGAAACTTTGTACGTAATCGTTGTTGTTTTTTTC |
| | | TCTCAGAAGCTTATAGGATATCTGCTTTTTCCCTTCCTCCTTT |
| | | AAATTCTTATAGGTGCTCCATTACCTACAAGATGAAGACCCT |
| | | TCTCAAACCGTCCTCAGGGTATTTCTTTTTCTTTTTTTCTTTCC |
| | | TTTTTTTTTTTTTTTTGAGGTAGAGTCTCACTCTGTTACCCAA |
| | | GCTGGAGTGCAGTGGTGCAATCTCAGCTCACTGCAACCTCTG |
| | | TCTCCCAGGTTCAAGCAATTCTCCTGCCTCACCCTCCTGAGT |
| | | AGCTGGAATTACAGGCACCCCCCACCATGCCCGGCTAATTTT |
| | | ATTATTTTTAGTAGAGACCGGGTTTCACCATGTTGGCCAGGC |
| | | TGGTCTCGAACTTCTGACCTCAGGTGCTCCGCCTGCCTTGGC |
| | | CTCCCAAAGTGCTGGGATTACAGGCATGAGCCACCGCACCT |
| | | GGCCTATTTTAACCATATAATAGCTCCTCCTGAAAATGATCA |
| | | ATGCTTAAGTCACAGAAAACTGTTGTCGCATGTTTACTCCTC |
| | | CCTGCCTTTGCACAAACTATTCTCTGTGCTGGACCTGCCTTCC |
| | | TCCACCAACCAGTCAAAATTCTACCTGTCCTTCCA |
| 22 | HLA- | CACTAGGTGGGATCATGAGTTTGCTTTGGACACCCCAAATTC |
| | DOB | TAACTATTTCTTTTGTTTCTTACATCCTTTCCCTCTTCCCCAGC |
| | | CCCTTCCCCTCATGTTACACCTCTTGCTGGTTTGAGACGTCAA |
| | | TCACCACTGAGAAAGAATTAAACCAGTATTTTGAGCTGGCA |
| | | AAATTCTTAGCCTAGTACAATTCCTTCAATTAAACTGTAGCT |
| | | CAACAATGTGTTCTCTAAAAGTATGATTTAATATTCTACCTA |
| | | GAAACTCAGAATATTTTTCATAACTCCTCCAGGGCAGTGTGG |
| | | ATTATGTTGATGTGTTAGGAGGAAGTAGAAGGAAGAAAATG |
| | | AATTGAGATGTACGTTTTACTTCCATGTCAAAGTCACCAAAA |
| | | TAGCAGTTGAAGGGATATTTGTGTCAGGCAATAGGGAGAAT |
| | | AATAATTTTATGCCACCCTCAAAAAACAACCACACATACACC |
| | | CAAATTCTCTCACTCCTCAGAGAAAGGGGAAGGAAAGAAAA |
| | | GAGAGAAGCAAAATATGAGCTTGAGTGAAAAATCACAGAG |
| | | GAGCCTGTAGCTATTTAGGGAGGGCTGCTGGCTGAAGTCAG |
| | | GCAGGAAAAAAAGATCAAAACACCCTGCCCTATTTTTCAGG |
| | | CTCGTTCAAGTAGAAGACAAAAACATAAATACAGGAGAAAG |
| | | GAAGAAAACCACATCTTTTTCCTCTTGTCTCCCCAGAACTGA |
| | | ACAGTGTCTCTGATAAGCCCAGGCCCTTCTATCGTAGACACT |
| | | GACACTATGCACAGAAAATGACTCAAAAACGCTTCTAATGG |
| | | GGGTGAATCTGATGCTTCAGTTTATTTAAGATGTACCAGAGG |
| | | CCATCTAAGGAGATCCATAGCTTGCTAACTGAAGCTTATTGC |
| | | TTTCTCTTCTTAGTTCCCATGCCAGGCTCTATGCCATTCCTTG |
| | | CTTGTCATGTGAGATCACTGATTTCCTTTGGTTAGGTAGGAT |
| | | ATGATTTCCTTACAGAGATCTGTCCTGACACCAAACAGCTAT |
| | | GATGAAATTCCTTGGTTATTTTCCCTTTTTGTGTACCTTATCA |
| | | TTACCGGAATGCTAAAGCTGTAGGAATAAAGTTTCCCTGGCT |
| | | TCCCAAGAAATACAGTGTGACAGAAAAAGTATAGCCTAGGG |
| | | ACTTACTAGTTATGTGAACTTTGGTTCATTCCATCCTTACCTC |
| | | ATCTGTAAAAGGGAGATCATGATAGTATCTACTGCAAAGGG |
| | | TTTTGTGTGATGATTCTCTCTCTTTCTCTCTCTCTCATACACA |
| | | CACACGCACACATTTATATAATGCTTAGAAAATGAACTCATA |
| | | ATAAGCAATTGACAAACATTAGCTATTATTATGTAGGCAAGT |
| | | CAGATTTTAGAGTTTGTGAGCCTTAGACACATTTACAGAGAA |
| | | GAAAGAGCAGCCCTCCTAACTTTCTGGTCCAGCGCCATATCC |
| | | TCCACTTCCTCCCCATCCCCACATCCCTTGCCATTTATCAACC |
| | | CCCTCTCTCCTCTAAATCTAAATACAGGCCCCCGTTTGATCC |
| | | ATATTGTCTAGGCCCTTCCTCTCCTCTCCACATGGCCCTTCCT |
| | | TCAGCTCTGAGGGAAGCTGCAGAAGCCAGCCATGGTGCTGT |
| | | CTACAAAGAAGGGGACACACGCCTCTTCCACCCGCTCATGCT |
| | | GTTACTGCATCTGATCATCTCCGTGCCGTGTCCTTGTTCACTT |
| | | AGCCTGTGTTGAGTGTTTGTCTCCATTTCCAAATGCAATAAA |
| | | ACATCTGGGAAAGACTAAGGTAGGTGTGGGCAGGAAGAAG |
| | | GGAGGAAGTTAGACCCAGTGGCTTGAGTGCCCTCTGATGCCT |
| | | CCTTATCCTCGGCTCCACACAAGCCCTCGCCAGTGTGAGCTC |
| | | CACAGCCATCCACCTGGAGGAGGAGTACTCAAAACCAGGGT |
| | | CAAATGCCTTGTACTCGGGGGTCTACCAGTAAGCCTGTGGCC |
| | | AGCCTTCCACTTCATGAATCGTCACATTTACATGAGAGATGT |
| | | GGAGGGAGAGGGGTCAGCCTCCTAGCTCCTGTCCTGTAGCA |
| | | GTTAAGTCAAGTCAGGCCAGGCTGTGGTAACCAAGCCAGTA |
| | | CCTTCTTTAAAGAACAGTTGTTTCTCATTCATACAGAGTGTG |
| | | TTGTAGGTGTGAGTGATTCCCTGGGGCAGCTGCTCTTCATGG |
| | | TGACTCAGCTAATTAGACTGCTTTTCTACTGGGATTGTGCCA |
| | | TCTCAACACAAGGCTGTCTTGGTTGACTTGGTAGGGGAGGA |
| | | GGAGAAGAGTCATGCACGAGGAGTTAAATTCTTTGACCAGG |
| | | AACCTTTTGTTCACTTTCCATTGGTGAGAACGAGTCACACAG |
| | | CTCCAGGAGAGTAGGAAGTCTAGTTTCTCGTTTTCCATGGGC |
| | | CTAGGAATTAGAGGATGAACACTAGCGATGTCTTCCACGTCT |
| | | TGCTCTCTTTTTCTTTACCTGTTATAATCTCCCATGGAGAAGA |
| | | AATTATTTTTCTGATTGGTCTGATCCAGGCCTCTAACCAAGG |
| | | GCATTTACTAAGGGTCATAGAACAGTAAAAGGTTGCACAAT |
| | | TGCATCTCACCTCTCTTTTCTGAATCCTGCTTAATTAAAACTT |
| | | TAAAAACTCACTTACAGACCACTTCTTGCTCTTCTTAGTTATC |
| | | AAATATATTTTCTTCCAACTCCTTTTTGACAGATCTTGCCTTG |
| | | TTCTCAGCTGACTCCCTTGCAACCATCTAAATGGAACTTCCT |
| | | CATTCCCCTTTATCCCCTGCTAATCGTCCTGTCTTTATTCTTTT |
| | | ATCTGAAGAAGTGTACCCTTCTTCCCCCAAGCCTTATGCTCT |
| | | CTGTCTTAGTCCATTTGTGTTAGTGTAACAAGATACACGAGA |
| | | TAGGGTAATTTATGAAGAACAGAAATTTATTTCTCACAGTTC |
| | | TGGAAGCTAGAAGTTCAAGGTCATGGCCCTGGAAGATTTGA |
| | | TGCCTTGGCCATCGACCTGAAAAAAGTCACTGAAGTGAACTT |
| | | GTGGTTACCATGATACCCTGGGTTCATTCTTAACATGCACAG |
| | | TATAGGCAAATCAGGGGCACATTTCATGCTGAAAGATGATG |
| | | TGAAATGATGAGTAAACATTGCCCTTTAAATAATAATAACAT |
| | | AGATGGTGAATTACAATTTGCACTTTTGGTTTTATTTGCTTAA |
| | | AGATAGTGTTTTACTGTCTAGGTTTTACTGAAAGAATACTTA |
| | | ACTAAACACTTGAGTCAAATCATTCCAGTGTAACAGTCCTCA |
| | | CAAGGAGGAAGGCAGAAGGGCAAAAGTGCCAAGCTATTGC |
| | | CTCCAGCCCTTCTATTTTTATTGATTGACTGATTGATTGATTG |
| | | AGACAGGGCCTCGTTCTGTTACCCATGCTGGAGTACAGTGGT |
| | | GCATTCATGGTTCACTGCAACTTTGAACTTCTGGGCTCAAGT |
| | | AGTCTTCCCACCTCAGCCTCCAAAGTAACTAAGACCACAGGT |
| | | GCATGCCACCATGTTTATTTCTTTAATTTTTTAAATTTTATTT |
| | | GGACATGGGGGTCTCACTATGTTTTCCAGGCAGGTCTCTAAC |
| | | TCCTAGCCTCAAGCAATCTTCCTCCTCAGCCTCCCAAAGTGC |
| | | TGGGATTACAGGCATGAGCCATCATCCCTGCTCTCTCTAGCC |
| | | CTTTTTAAAGTCTCTAATCCCATTCAGGAGGCCTCTGCATTC |
| | | ATGACTTAATCACCTCCTAAAGGGCCCACCTTTTGATACTAT |
| | | CACATTGGTGATTAAATTGCAACACTTGAATTTTGAGTGACA |
| | | TTCAGACCACAGCATTTTGGAGAAAATGTCCCCTTTCTTCTC |
| | | TTTTTTCTCTTCAGTTTCTCCTTTCTACAGATACCTTGCACTTC |
| | | ACTTGCAACCTTGTTGAAGTCTCTCCTGTCTTAAAAGTTTCTT |
| | | TTCCCAAAACACCAGAACTCATTCCTTCTGTCTAACTGTAAC |
| | | TTTGTACTTGTTACCAACCTCTACCCATTTCTACCCCTGACTC |
| | | CCCAGCCTCTGGTAACTCCTAACGTACTCTTTACTTCGATGA |
| | | GGTCAACTTTTTTAGATTCCACATATGAGTGAAATCACGTAA |
| | | TATAGTCCTTCTGTTCTTGGCTTATTTCACTTAACATAGAGCC |
| | | CTCCAGGTTCACCCATGTTGTTGAAAATGACAGGATTTCATG |
| | | CTTTTTTATGGCGGAATAGTATTCCATCATGTATATATATCAC |
| | | ATACTAATTACCCTGATTTGATCAATACACAATGTATAGGTG |
| | | TGTTGGAACATCATGCTGTACCCCATAAATATGTACAATTAT |
| | | TACGTGTCAATTTAAAAAACCCAAGAAAAGTATTTTTTCTTA |
| | | GTGTTGTTTTTTTCTAAAGTGTATCCCATTTTTTCCTTTGTTTT |
| | | CACTTTCAAAATTCTTGAAAGACTGTTTTAATCTGCGGCCTTT |
| | | GTTTTCTTGTTTCCAATTCATTCTTTTGAAATATGGCTTCTAC |
| | | CTTTAACACTCTGCTGTCCTCTTGAAGGTTATTGGTGCCCTAC |
| | | TAACCATAAAACACAATAATGCTCTCTTAGATTGTATCCTGG |
| | | TGAGCCTTTTTGCAAAATTTTATTCTTTGAGAATTCTCTTTCC |
| | | ATGTTTTCTTTTCTTTTCTTTTTTTGTGGTCATTTTGGCTTATA |
| | | GGAAATCGTACTCTCCAAATGTTCTCTATGTCTTCTATTTCTT |
| | | CTCTGATAATCCCTTTACTAAATCTTCTTGAGGCATATGCTTC |
| | | AGAATGCTTAATTTACATACTCTTAATTCCTTCTTAATTTACA |
| | | CACTGCCTGTCGGCAATGTCAACACCCAATAAGAAGGGAGA |
| | | CCTATTGGTCAGAACAGGCCAGGGAATAGAAGAATACATAA |
| | | TAAACAGTCTGCCTAGTTCTTCTAGGGCCCCATAATATGTCA |
| | | AACATATATTTTTACTTCTTCTCCCAGCCCATTTTTAGTATAC |
| | | CTAAATTACTGTCAGTGATTCTGCAGGCAAACAATGGTTGAG |
| | | TTGTATGACACAACTTTGTGAAAGTATCCTACCCAGTACCTG |
| | | ATGATGCAAAACTCTTCTATCTTGATTGGTTGTCAATCTGAG |
| | | GAGTTTCCAATCCTGGGGAAGCCAGAAAAACAGCGATTTAT |
| | | ACTCTTAATGGGTACTTTCTGACTGAATTTTATGAGCTCATTC |
| | | TGAAGAGGCTGACGATTTTACTATCTCATTTTTTTCCTTTCTC |
| | | CAGAATGGGTTCTGGGTGGGTCCCCTGGGTGGTGGCTCTGCT |
| | | AGTGAATCTGACCCGACTGGATTCCTCCATGACTCAAGGCAC |
| | | AGACTCTCCAGGTAAGAACAGAGCAATTGTTTTTTTCCAGTG |
| | | TGTATGCAAGAATTGGCATGGGGGAGTGATGCCTTTCTTTGT |
| | | AAGTCCAGGCCACAGACCAGACTGGAAGTGGCTTTTGGTTTC |
| | | AAAGAACAGTGTTCTTCCCTTTGGCAGAAAGGTACGCCTTGC |
| | | CTCTTTACATGGGATGGACTTCATATACCAGAGCCACCTATT |
| | | CAAGGGGTAGGGAGGCAGGAAGAGGGAAACATTGTGTCTTG |
| | | TTTAGGATCCTTATTGTGTGTATCAACCTCAGTCAGTGCCTG |
| | | GGCGTGTTGAAGGCCTTGGCTTGGGTTCGAGCCTGCTGGGAG |
| | | AAACAACCTGCAGTAGGCTGGGTCACAGAGGCAATCTGTGA |
| | | TTTTTTGGTCAGGACACGGAAACAAATCTCAGTTGGGGTATA |
| | | TGTGGACAAATGAAACTGGAAACAAAGGTTGCTCCTTCTGTC |
| | | ATTTATTAAGCCACTATTATATTGTCAGAATTGTACTAAACA |
| | | GTTTTGAGAAGTAAGAGAAGTTGAATAGAATACATTGTCCTT |
| | | GTCCTCCGGCTACCAGGTACAAGTTACTTGTCACTGTTATTTT |
| | | TCTAGCACAGGTGACAGAATATGCAGCCATGAAGCAATGTG |
| | | AGATGAAAGCACATATTAATGAGCAGAAACAGGATGTAATG |
| | | TGCTAAGAACAGAATCCCCTTTGCATGTTAGTTTCATTAAAT |
| | | ACAAAAGAGGAACAAACCTGGCCAGGAGAGATCATTATTCT |
| | | CAGAGAATAGAAACCGCCCTGAGTTTATAATGTCCATTAAA |
| | | CAATACAACTGAAAAAAAAATCAGCACAGATGTTAAATGAT |
| | | GATGAAAAATTCAGATTTCCCCCCTGGTTTAGACTACTAGAG |
| | | GAAATAGAGAAGAGTATACATGCTGAGAAATTACAGGCTGG |
| | | AACTTCATCTGAAATTAGCTACTGAGTGAGGGATAAGTGGG |
| | | GTTCACCCAGGAAGGTCATTCTTATGGCTCAGTTCAGAGTTG |
| | | GAGGAGGCTTCTGAACTTAGAAAGGAAAGTAAATTACAACC |
| | | CAACATTAATAGCAATTATCTTTCAAGTCTTGACTTAGATGC |
| | | AATGTCTTCAGGACGTCCTTCCTGACTTACCTACATTATTAA |
| | | CTCCATTTGAATTTCCTTTTTATTGTAGTTGTTGTTCTTAAGT |
| | | GCATAGGATTGGTTTAATTTTACCCAATGAGTTCACAGCACA |
| | | TTGTAATTATTGGCAGTAGTGCAAGACTCTCTCGTCTCTTCTC |
| | | TTGCCTCCGTTCTCATTCTCTCCCCTCCCTAGAGAATCCATTC |
| | | TAAACGTGTCTGGTAAGTCTCTAAGTATGAGAGTGGCTTTTA |
| | | GAAATATGTAGCATTATTGCTCTTTATGTGTTTTTAAAATTTA |
| | | ATAAATGTCATTCTCTGTTGAATCCCATTCTGTTTCTTTTCTC |
| | | ACTTGACACTGTGCTTTTTGAGCATACTGAGGTCAAAGGCCT |
| | | CCTCTTAGATCCATGTGGTCTGACGTAATTTACCAGGCATGG |
| | | GTTTTCCCAGAGGAGGGGGCTGGTTCATGGTTTTGGTTTTGG |
| | | TTTTCCAGAAGATTTTGTGATTCAGGCAAAGGCTGACTGTTA |
| | | CTTCACCAACGGGACAGAAAAGGTGCAGTTTGTGGTCAGAT |
| | | TCATCTTTAACTTGGAGGAGTATGTACGTTTCGACAGTGATG |
| | | TGGGGATGTTTGTGGCATTGACCAAGCTGGGGCAGCCAGAT |
| | | GCTGAGCAGTGGAACAGCCGGCTGGATCTCTTGGAGAGGAG |
| | | CAGACAGGCCGTGGATGGGGTCTGTAGACACAACTACAGGC |
| | | TGGGCGCACCCTTCACTGTGGGGAGAAAAGGTGAGCTGGAA |
| | | GCTGAGGTCTGGCGGGGCTCAGGAATGTCCCCCATGTGAAC |
| | | CTGGCCATGGCTCTTCTTTCTTACAAGCAATTTTCTGCTTTAG |
| | | GATAAATGGTTGTCTGTGTAGATGTTCTGGCCCCAGCTGTGA |
| | | TATATTATCCTCACAAGTCAGCCACTGTGATCTTGGTCTCAG |
| | | ACCCCCAAGGTTCTCAGGGACTTCGAGGGCTATTGTGCCCTC |
| | | AAAGAGAAGCAGTAATTGTGGGAGTACCTCAGAAAGTCTAA |
| | | ATCCTCCTGACAGGCATTGACATACCCTGTTACTGATCTTGG |
| | | GGGCTGAGACTTGCCTATACTTTGTGTTCACTTGGGTGATCT |
| | | GGGAAAGAGATTAGACATAGTGATAGTCCCTAAAGAATCTC |
| | | CTGTCCCAGCTTGGTGGTTTTCTTTCACGGTGTCTCATTTTTC |
| | | CTCCCTTCCTAGTGCAACCAGAGGTGACAGTGTACCCAGAG |
| | | AGGACCCCACTCCTGCACCAGCATAATCTGCTGCACTGCTCT |
| | | GTGACAGGCTTCTATCCAGGGGATATCAAGATCAAGTGGTTC |
| | | CTGAATGGGCAGGAGGAGAGAGCTGGGGTCATGTCCACTGG |
| | | CCCTATCAGGAATGGAGACTGGACCTTTCAGACTGTGGTGAT |
| | | GCTAGAAATGACTCCTGAACTTGGACATGTCTACACCTGCCT |
| | | TGTCGATCACTCCAGCCTGCTGAGCCCTGTTTCTGTGGAGTG |
| | | GAGTGAGAATTAGTTTCTAGTACTCTCTGGGCCTGACTCAGG |
| | | ACTATACTGACTCAATACAGAGCCTGTGTCACTTCTGCGTTT |
| | | ATCTTGGTCACAACATGAATTATTCTTTCCCTTGATCTGGGA |
| | | CAGTCACAGAAACCAGAGTCCTTGGGTTAGGGTGGGAGAAA |
| | | ACATGGCAGATATCTATCCTCATATCTTCCAAGAAATGAGGA |
| | | GATCTAATCACCTCATTATGTGCTTCCAACCCTATGAACTGG |
| | | TGTCCTCTAATTCTTTGGTCTTAGTATTTAGGAGGCATTCTTA |
| | | TGGGCTGTGAGAATCTGTAACCGATGGGGGGTAACTCCATG |
| | | GGTGCCAACTTTGGTTTCGAAGAACCTTTTCTAAATTTATTTA |
| | | TTTTTCTCTAGCTAGCATTGGATTTGGTGTCTAGTACAGATTC |
| | | TGGGATTCCAAGAAAGTGCTTTAAATATTGGGATATTTTTAC |
| | | TAATTTAAAGACCTGTTTCCCATAGGAGCTCAGTCTGAATAT |
| | | TCTTGGAGAAAGATGCTGAGTGGCATTGCAGCCTTCCTACTT |
| | | GGGCTAATCTTCCTTCTGGTGGGAATCGTCATCCAGCTAAGG |
| | | GCTCAGAAAGGTAATGAGCCTGTGAGGAGTGCCCTGCCACC |
| | | TGTCCCAGACCTTCCCCACTCCCACCTTCCCTAACGTCAATG |
| | | ATCTGAGGCAAGGAAAGCTGATTGTGCCTCTCAGGGATCAC |
| | | CGGGATAATTTTTTTCTGAAGCTAGAAATGGGATAAGCAGA |
| | | GAGAGTGCTGACCTTGCCAGCCATTTGTTCTTCCCTCGGGAT |
| | | AATCATATTGGGTCCTAATTGGGGCAATCCATTCTTTTCTCG |
| | | ATTTCTTTCCAGGATATGTGAGGACGCAGATGTCTGGTAATG |
| | | AGGTAATGTCTCTTTTTCCTTGTCTTTGAGTGGCAGATCATTC |
| | | TCCCGGTTCTTTGGCCAGAGGGAGATGACATGGGGGTAGGG |
| | | AGGAGTAAGGTTGCTGCTGTCTGGATGGGACTGTCCCCTGAG |
| | | TCTCTGGAACGGCTGTGGGGGGTGGTGAGGCTGCCTCCTGA |
| | | GACCTTCATCACTGTGCCTCCAGGTCTCAAGAGCTGTTCTGC |
| | | TCCCTCAGTCATGCTAAGGTCCTCACTGAAGCTTCTCTCTCTG |
| | | GAGCCTGAAGTAGTGATGAGTAGTCTGGGCCCTGGGTGAGG |
| | | TAAAGGACATTCATGAGGTCAATGTTCTGGGAATAACTCTCT |
| | | TCCCTGATCCTTGGAGGAGCCCGAACTGATTCTGGAGCTCTG |
| | | TGTTCTGAGATCATGCATCTCCCACCCATCTGCCCTTCTCCCT |
| | | TCTACGTGTACATCATTAATCCCCATTGCCAAGGGCATTGTC |
| | | CAGAAACTCCCCTGAGACCTTACTCCTTCCAGCCCCAAATCA |
| | | TTTACTTTTCTGTGGTCCAGCCCTACTCCTATAAGTCATGATC |
| | | TCCAAAGCTTTCTGTCTTCCAACTGCAGTCTCCACAGTCTTCA |
| | | GAAGACAAATGCTCAGGTAGTCACTGTTTCCTTTTCACTGTT |
| | | TTTAAAAACCTTTTATTGTCAAATAAAATGGAGATACAAAAA |
| | | ATGTACATTTTAGTGAATTATTTAAGAAAAACCCCTGTAATC |
| | | AAGTCAAGGAACAGGACTTTGCCAGCTCCAGCGGAAGTCTC |
| | | TGTACGTCAGGCCAATCAAAGCCTCTCCTTCCCCTCGAAAGT |
| | | GACCATATCCTGATTTTATTGTAACCTCTTTCATGTCTTTGTA |
| | | GTCTAGTCCCCCAGGTATGTGTTCCTGGACGCCACAGCTTAG |
| | | TTGTCTTTTACGTCTCTCATACTTCACTGGTTTCTCCCCCATC |
| | | AGCCTTTTTTTCCCCCTTATGATTAATCTGTTGAAAAGCTCAA |
| | | TCCATTTGACCTGTAGAGTTTCCCACACTCTGGATTTTGCTGG |
| | | CTGCGTACACATGGTGCAGTGTAACACATCCTCTGCCTTCTG |
| | | TGCCTCCCGCAAATTGGCATCTGGATGCAGAGGCTGGATCA |
| | | GACTCTGGTTCAATCTTTTCCTTTGGAAAATCTATACGTGGT |
| | | GTTGTGGCCTTTCACCATGAGAGACATAATTTCCAGCTGTTT |
| | | CTTTTTGTGATGTTAGCAACCATTGATACTAATTGCTTAGGTC |
| | | TGTTAATTTATTGGGGATTGCTAAATGGTGGTATTCTGTCATT |
| | | TTTTCTTCATTTATATGCTTTATTAATGCTACAAAGAGACAGT |
| | | TCCCTTCATTTACTAGTGAAGAGACACTTCCCTCCACTTACT |
| | | AGTGGGCTACTTAGTGGTACAGTGGAAAGGCAGGATCAGTG |
| | | TTTGACTCTCCTTTTATTTACCAGTTTTCAAGTTAGCAAATTG |
| | | GTTTCCTACTATTAATAAAACCTACAGATGGCCAATCAGGTT |
| | | TTACTTTTCATTCTTTAAATAATTATAAACAGAAGGATTTAA |
| | | ATGTTTGATAGGTTTTACTCTATTGCAATTTTTATGCTTGTTA |
| | | AAGCTTATATCCTACATTTTTGGTGAGTGAAAACTTCTTCAA |
| | | GTGGCTAAAAATGAAACTGAGTTCTTTTGACGCAAATTTCTT |
| | | TTGATAATTTCTTTGTTATCTGGGATGACAACATGTTTCTGGT |
| | | TCATCTTGTTCATTTCTTGGCCAGACCTGGAGGTAGCCATTTT |
| | | TCCAGAAATCCCTGGTTTAGTTTAGTGGGAAATGACATTTAA |
| | | GACTATAATCCAGCGTTAGGGTGTTTTGTCCCACTGAACCCG |
| | | CTGCTGGAGTCACCTCAAAAGTGGCGTCAAGGGAGTTACCA |
| | | CAAGGAAGTTCCTGAAACCATTCTGAGATGGTGTGGGTTAG |
| | | GATTCAAAGAAAGAAGCACTAAATGCCAGGGTGATCAGTCC |
| | | AAAACATTTGTTTAGGGAATTTACCTACAGAGGGCTTCAGGA |
| | | GTCCTTGCAGACAGCAAGAGAAAAGGGATGTTCTGCCTAGG |
| | | TATGTCTGCAGCGAGGGGGTCAGGGTATGGAGCTTATATGA |
| | | AGGTTTAAGGAACCTGGCTCAGGGCTGGGACAAAGTTTCAG |
| | | TGTTTAGAGAAACAACCTAGATACGTTTATCAGTGCCTGGGA |
| | | GTGTTCAAGGCCTTGGCTTGGGTTCGAGCCTGCTGGGAAAAA |
| | | CCTGCAGCTGGCTGGGTCACAGAGGCATTCTGTGATTTTTCG |
| | | GTCAGGACATGGAAAGAAAGCAGGGAGAGGGGCAGTGGGG |
| | | GAGCCTAAATAGATAGGAATGGTCATTGTGTCCAGCCTGTTC |
| | | AATGGCAATAAAACATTTATACATGTATACATTTAAAAAAAT |
| | | CAGTCATCCAGGCACGGTGGCTCACGCCTGTAATCCCAGCAC |
| | | TTTGAGAGGCCGAGGCGGGCGAATCACGAGGTCAAGAGATT |
| | | GAGACCATCCTGGGCAACACGGTGAAACCCCGTCTCTACTA |
| | | AAAATACAAAAATTAGCTGGGTGTGGTGGCACGCGCCTGTA |
| | | GTCCCAGCTACTCGGGAGGCTGAGGCAAGAGAAGCGCTTGA |
| | | ACTTGGGAGGTGGAGGTTACAGTGAGCCAAGATTGTACCAC |
| | | TGCACTGCGGCCTGGTGACAGAGCAATACTCCATCTTAAAA |
| | | AAAAATCAGTCTTCTCTATATTACTTCTGCATCTCCT |
| 23 | HLA- | GTTCGTTGCAACAAATTGATGAGCAATGCTTTTTTATAATGC |
| | DQA1 | CAACTTTGTACAAAAAAGTTGGCACCATGATCCTAAACAAA |
| | | GCTCTGATGCTGGGGACCCTTGCCCTGACCACCGTGATGAGC |
| | | CCCTGTGGAGGTGAAGACATTGTGGCTGACCACGTCGCCTCT |
| | | TATGGTGTAAACTTGTACCAGTCTTACGGTCCCTCTGGCCAG |
| | | TACACCCATGAATTTGATGGAGATGAGCAGTTCTACGTGGAC |
| | | CTGGGGAGGAAGGAGACTGTCTGGTGTTTGCCTGTTCTCAGA |
| | | CAATTTAGATTTGACCCGCAATTTGCACTGACAAACATCGCT |
| | | GTCCTAAAACATAACTTGAACATCCTGATTAAACGCTCCAAC |
| | | TCTACTGCTGCTACCAATGAGGTTCCTGAGGTCACAGTGTTT |
| | | TCCAAGTCTCCCGTGACGCTGGGTCAGCCCAACACCCTCATC |
| | | TGTCTTGTGGACAACATCTTTCCTCCTGTGGTCAACATCACA |
| | | TGGCTGAGCAATGGGCACTCAGTCACAGAAGGTGTTTCTGA |
| | | GACCAGCTTCCTCTCCAAGAGTGATCATTCCTTCTTCAAGAT |
| | | CAGTTACCTCACCTTCCTCCCTTCTGCTGATGAGATTTATGAC |
| | | TGCAAGGTGGAGCACTGGGGCCTGGACGAGCCTCTTCTGAA |
| | | ACACTGGGAGCCTGAGATTCCAGCCCCTATGTCAGAGCTCAC |
| | | AGAGACTGTGGTCTGCGCCCTGGGATTGTCTGTGGGCCTCGT |
| | | GGGCATTGTGGTGGGCACTGTCTTCATCATCCGAGGCCTGCG |
| | | TTCAGTTGGTGCTTCCAGACACCAAGGGCCCTTGTTGCCAAC |
| | | TTTCTTGTACAAAGTTGGCATTATAAGAAAGCATTGCTTATC |
| | | AATTTGTTGCAACGAAC |
| 24 | HLA- | GACATGCACACACCAGAGAAGATTCCAATTTAGTGTCTTCCC |
| | DQA1 | TCTCTTCATAGAACAATTCCTCAAGTCCACTCTGAGTAGAGG |
| | | CTGCATCACAACAAGGGGATTGCCCTGTCTCCTTCCAGGGCT |
| | | CTTAATAGAAACTCTTCAACTAGTAACTGAGATGTCACCATG |
| | | GGGGATTTTTCTAATTGGCCAAAACCTGACTTGGCAGGGTTT |
| | | GGTTTGGGTGTCTTCAGATTTCCTTGTCTTGAGGTCCTCACAA |
| | | TTACTCTACAGCTCAGAACAGCAACTGCTGAGGCTGCCTTGG |
| | | GAAGAAGATGATCCTAAACAAAGCTCTGATGCTGGGGGCCC |
| | | TCGCCCTGACCACCGTGATGAGCCCTTGTGGAGGTGAAGAC |
| | | ATTGTGGGTGAGTGCGTGAGTGAGGAATGTTCTCTGGAGCTG |
| | | AAAAACAGTAAATTGAAGGAAAAGAGAGAAAGCGATTTGC |
| | | AGAGAAATTGTAGAGATTTCCTAAGACCCCTTTCAGTATTAA |
| | | GAGAATTAAAAATTATAGCTGTTCCTCCTTCAGGAAACCAGA |
| | | GCCCCAACCTACTCTTTTTGTTATGTATGCTTTTGTGTTCACT |
| | | AAGGATGCTATTCTGTTTATATTATATTCAGTGACTACAGCC |
| | | TGGAGGTCTCTATGTCATTCCATCATGATTGCCTCAAAAATT |
| | | AGTGAGGTTTCCATCAGTGGATAATTTTTTATTATTAAAAAT |
| | | GTATGAAGTGTCATTCTCAAATTTCCCTGAACAACTTTTGAA |
| | | GATTTTCGGATGTCTCCTGTAGTAGATCTTGGGGTCGTTCCA |
| | | TCAATTATATACTCTATAGATATTAAAAAAGTTGCCCGTTTC |
| | | TTTCTCTCAGACTTACTCACATTTCCACATGGGAACTGGCAC |
| | | AGGTGGGGAGTAGGTAAAGGAGTCCAGCAGGCTGAATGCCT |
| | | TCAACAATCATTTTACCACATGGTCCTCACTTACTCTCAGCT |
| | | GCCTCATATGTGTCACCTCACAAATAATCAAATAAAATGGGC |
| | | ATGTAGCTAAGCTTTGTAAATAGTGAAAACATGGATGTCAAT |
| | | TGTTTTTACATATTTCTATTACAGGTATAGCTTCACATTTCTT |
| | | TTCTTTAGCAAAATAAGGGATCCTTTTAGTTTAAAATTGAGA |
| | | AGTAGAAAAAATTGGTAAATTAAATCATTTTATTCTCAAATT |
| | | ATCAACCCAAATTACCTGTTCTTCACCTCATCTAATAAAGTC |
| | | CTATAAAAAGAAAAGTGGGCCAGACATGGTGGCTCATGCCT |
| | | GTAATCCCAGCACTTTGGGAGGCCGAAGCAGGAGGATCATT |
| | | TGAGCCTGGGAGTTTGAGACCAGCCTGGGCAACACAGCAAG |
| | | ACCTCATCTCTACCAAAAAATAAAATAAAAATTAGCCAGGC |
| | | ATGGTAGTGCATGCCTGTGGTGCCAGCTACTCAGAAGGCTGC |
| | | AGTGGGAGGAGCACTTGAGTCCAGGAGGTGGAAGCTGCAGT |
| | | GAGCCATGATGGCACCACTACACTCCAGCCAGGGCAACAGA |
| | | GAGAGACCCTGTCTCAAAAAGAAAGCGGAAAGAAAGAGAG |
| | | AAAGGAAGGAAAGAAGGAAAGAAGGAAGCAAGGAAGGAG |
| | | AAAGGGAAGGGAAGAAAGAAGAAAGAAAGAAAGAAAACA |
| | | GAAGGAAGGAAGCACAGATTAATTATTTGGTCTCTTAGTCTC |
| | | CTCTGCCTTTGTCGTCCATCTCTTCCCACCTCTCTTCATGCAT |
| | | TCCTTTCTCCCTCTTCCCTTTCAGGATCCATCTCTGACTCCCT |
| | | GCTCCTTTATAGAGATGGACATGAGTTTGTAAAACAAAAGTT |
| | | GAAAAGTCAGATAGTTAAAAGGGGAAGTAAACTGGAAGGT |
| | | ACTCTAAACTTTCACAACCTTATTAACCGTGGCAGCTCCCAT |
| | | TCTGATTTTGTTCAGCAGTGGAAGTTTCACCCTCTCCTCCAG |
| | | AGCGCTTGGCTTCTTTGTTCCAAATTTCCTTTCTTCAGCCTCA |
| | | CACCAGAGTGCCCTGGTCAGGCTCAGCTCATCCATTAGGCAC |
| | | AATGTGGGCAGTGCAGGGGAACCTCCATACTGTAAAGCCAC |
| | | ATGAGAATGTTTTAACTCCTTTTAAAATTATAAAAAAATGAA |
| | | ATTGTAGAGCCTAAGAAAATGTTTTAACTTTTAATTCAGCCT |
| | | ATATTATATTGTCTTTATACCAATTCAGTCATAAAATATAATT |
| | | TTCCATATTTTTATGGAGGAAGGCGTCCACACAAGCAAGAGT |
| | | GCTTGGGGCTCACATGTCAGAACGCATCCCTGATCATGGCTG |
| | | ATCCTGACCTTCGTGTGGTTCTGCTAACTATGTGCCTGTCAGT |
| | | CTTCCCCAAAATCTATGTGGTCCTCAAATATAACAACTGTCA |
| | | TTCAATACACATGTTTGAGCACCCAGTGAGCTAAGTTTTAAG |
| | | GATTCAAAGATGAAAAGTCATGCTGTCTCCCCTGCAGAGGG |
| | | TGCTCAGACTAGTGATGGAAACAGTATGGGATGAAAGAAAG |
| | | CAGAAGGCCATTGCTGAGCAGGCAGTGGACTCAGCAGAGGC |
| | | TGAAACTATACAAGTGACTTGGTTCCAGCTGGGCCAGCAGG |
| | | ATAACCAGACGAAAAGAAGGATTGCATATATTCCATATATA |
| | | TTTATGTTTGAACAAAGAGTCAAGGTTTATTGCAAGGATAAG |
| | | GAGGCTTTGTTGGTGGCCTGTTAAGACCATCCAGCGTGGTCA |
| | | TACTGGATAGGGAAGAAGGTGAGCTGGAAGAGGGATAGAC |
| | | AAACTTGGATGGCCAGATGTTGAGATGGAGGAGCTGGAGGT |
| | | CATAACGTGGTCAAAAACATGTTGATGAGAGGACTTAGCTA |
| | | CAAAGTTGTTAACTTAAGCAGAAACCTCAAGGATTGATTTTA |
| | | TGATTTCTCCAGGAAGTCCTAAAAGATAATTTCATTTCAGGG |
| | | AGGAAAACAACAGACCACTGCAAAGACCAGGAACATGAAA |
| | | GGATAATGTAGTTTGGTTTGCTTGGCAGATACTTGTGAAAGA |
| | | TGTTGGACTGTAAGGCTGTCAATATCCTCCTCGCAGAACTTA |
| | | CTACAGTACATTGTATCTGCTCCCTTACCTACCTGACTCTCCC |
| | | ACTATTCAGTTTGTTCCTTAATGGTAGACCATGCCTGATTGG |
| | | TGTTTTACACTTCCCCTGCTATGTCTGATACTTGTGGATGCTC |
| | | AGAAAGTGGGGAAGGAAGGAAAGATACGATGGTAAAAGGC |
| | | TTACACATGTCTTGACCAGAATGTTCAGTTTGGCTCATTTGG |
| | | CTGGAGTCATACTGCATGGCTGCCATTCTGCTCTGGCATCCT |
| | | CAGAGAAGCACACTGCCCATTAGAGGAAAAAGGGTGAATAT |
| | | AAATGTTGAGTCAGAACACTGCAGACATTTAGTAACCTCCTT |
| | | CAGAGGAAAAAAAGGGTGGGGGGAATGACAGAAATCCAAA |
| | | AACTAGTAGAGCTTCCACTTTTTCATTTCAGAAGAAATCAGT |
| | | TACTCTCCTCTAAGGACCATTACTATTAACAAAACAGAGACC |
| | | TTAGAAGGAAGCATTATTTATTTATCATATATTTTGTAATGTT |
| | | ATTACCGTTCTTGTTATACTCTTTCTTATACCCTACCATTGTT |
| | | AGCAGAAATTATTTTAAATTAATAAGATCCTGCATGCTTTTC |
| | | CTTTTTCTAAAAAAAGAAAGATCTCTGTGTAGAATGTCCTGT |
| | | TCTGAGCCAGTCCTGAGAGGAAAGGAAGTATAATCAATTTG |
| | | TTATTAACTGATGAAAGAATTAAGTGAAAGATAAACCTTAG |
| | | GAAGCAGAGGGAAGTTAATCTATGACTAAGAAAGTTAAGTA |
| | | CTCTGATAACTCATTCATTCCTTCTTCTGTTCATTTACATTAT |
| | | TTAATCACAAGTCCATGATGTGCCAGGCACTCAGGAAATAG |
| | | TGAAAATCGGACACGCGATATTCTGCCCTTGTGTAGCACACA |
| | | CTGTAGTGGGAAAGAAAGTGCACTTTTAACTGGACAACTAT |
| | | CAACACGAAGAGGGGAGGAAGCAGGGGCTGGAAATGTCCA |
| | | CAGACTTTGCCAAAGACAAAGCCCATAATATTTGAAAGTCA |
| | | GTTTCTTCCATCATTTTGTGTATTAAGGTTTTTTATTCTCCTGT |
| | | TCTCTGCCTTCCTGCTTGTCATCTTCACTCATCAGCTGACCAC |
| | | GTTGCCTCTTACGGTGTAAACTTGTACCAGTCTTACGGTCCC |
| | | TCTGGCCAGTTCACCCATGAATTTGATGGAGACGAGGAGTTC |
| | | TATGTGGACCTGGAGAGGAAGGAGACTGTCTGGAAGTTGCC |
| | | TCTGTTCCACAGACTTAGATTTGACCCGCAATTTGCACTGAC |
| | | AAACATCGCTGTGCTAAAACATAACTTGAACATCCTGATTAA |
| | | ACGCTCCAACTCTACCGCTGCTACCAATGGTATGTGTCCACC |
| | | ATTCTGCCTTTCTTTACTGATCTATCCCTTTATACCAAGTTTC |
| | | ATTATTTTCTTTCCAAGAGGTCCCCAGATCTTCTCATGGCAAT |
| | | TGCTGAAATTTTATCATTTCTCATCTCTAAAATCACATATCCC |
| | | CATGTAATACAAGGGTCTTTCCATTATGCATTCATTAAATCA |
| | | TTCTAGGAGATGTCTCATCAACCTCCTACTTTATTAAACATG |
| | | CCCACAGAGAGAAGGGCACAGGAGTAAAGCAGAGGCAATG |
| | | TGTCATTGCTCCCAAGTAGAAGGTAAATAAGGCCTCTTTGAC |
| | | CAGCAGGAGAGGAAATGCTGGTAGGAAGACTCTTCCAGGAT |
| | | GTAATGCAGAAGCTCAGGGCAGAGCTATTCACACTTCACAC |
| | | CAGTGCTGTTTCCTCACCATAGAGGTTCCTGAGGTCACAGTG |
| | | TTTTCCAAGTCTCCCGTGACACTGGGTCAGCCCAACACCCTC |
| | | ATCTGTCTTGTGGACAACATCTTTCCTCCTGTGGTCAACATC |
| | | ACCTGGCTGAGCAATGGGCACTCAGTCACAGAAGGTGTTTCT |
| | | GAGACCAGCTTCCTCTCCAAGAGTGATCATTCCTTCTTCAAG |
| | | ATCAGTTACCTCACCTTCCTCCCTTCTGCTGATGAGATTTATG |
| | | ACTGCAAGGTGGAGCACTGGGGCCTGGATGAGCCTCTTCTG |
| | | AAACACTGGGGTAAGGATGAGTTTCACCATTTTTTGATGCTT |
| | | TCTTGTCTGTCAAGTTCAGAACTTCCTGCCTTTTACTCTATGT |
| | | CCCAAAACTTGTTTTCCACACTTCATGAGTTTCTTTTATCTTT |
| | | TTTTTTTTTTGAAAGAATTAAGCAACAAAAGCACAGATTTAT |
| | | TAAAAAAGAAAGTACACTCCACAGGGTGGGAGCAGGCCTGC |
| | | CACTTCATGGGTTTCTAATAACAGACTTCACTCTCCTCCCTG |
| | | AGCCAGGGGCCTTGAGTCTTTGCAGAGCCAACCCTCCACCCC |
| | | ATCCCATCCCTCACACATGCACATGAGCACACTCTGCATTCT |
| | | GACCTCAACAACTTCACTTCCACAGAGCCTGAGATTCCAGCA |
| | | CCTATGTCAGAGCTCACAGAGACTGTGGTCTGTGCCCTGGGG |
| | | TTGTCTGTGGGCCTCGTGGGCATTGTGGTGGGGACCGTCTTG |
| | | ATCATCCGAGGCCTGCGTTCAGTTGGTGCTTCCAGACACCAA |
| | | GGGCCCTTGTGAATCCCATCCTGAAAAAGAAGGTAAGTTTG |
| | | AGATTTGTTAGAGCTGAAGCTGCAGGAAGGAAAGTGGGAGG |
| | | AGGCTGTGGACATGAATGTGGTTGAAAGTTGTAGGGGAATT |
| | | GGG |
| 25 | HLA- | CACGGGCCGGCGGGAACTGGAGGTCGCGCGGGCGGTTCCAC |
| | DQB1 | AGCTCCGGGCCGGGTCAGGGCGGCGGCTGCGGGGTGGCCGG |
| | | GCTGGGGCCGGGCCGGGGCCGGACTGACCGGCCGGTGATTC |
| | | CCCGCAGAGGATTTCGTGTACCAGTTTAAGGCCATGTGCTAC |
| | | TTCACCAACGGGACGGAGCGCGTGCGTTATGTGACCAGATA |
| | | CATCTATAACCGAGAGGAGTACGCACGCTTCGACAGCGACG |
| | | TGGAGGTGTACCGGGCGGTGACGCCGCTGGGGCCGCCTGAC |
| | | GCCGAGTACTGGAACAGCCAGAAGGAAGTCCTGGAGAGGAC |
| | | CCGGGCGGAGTTGGACACGGTGTGCAGACACAACTACCAGT |
| | | TGGAGCTCCGCACGACCTTGCAGCGGCGAGGTGAGCGTCGT |
| | | CGCCCCGCTGCGAGGCCCACTCTTGGCCGGGGCCCCGAGTCT |
| | | CTGCGCTAGGAGGGACGAGGGGGGCGAAGCCTCTGGAACCT |
| | | GAGCCGCGTTCGTTCCACCCCAGGGGACAGGAGTTGGCGGC |
| | | GTGGGTGGTGGGGCAGGTGCATAGGAGGGGCAGGGACCTAG |
| | | GGGCAGAGCAGGGGTACAGGTAGAGTTGGTCAAACTGCCTA |
| | | GTTTTGCCCCAGCCTTCCCGTCCGTCAGCCTCGCCCTCTGCTG |
| | | TGCACGTTCTCGCCTCGTGCCTTATGCGTTTGCCTCCTCGTGC |
| | | CTTGCCTTTGCTAAGCAGTCCTCTCTGCCCCCAATTTCTGCCC |
| | | TCTTCCCCTGCCCGCCCGCCCCGCTAGCACTGCCCCACCCAG |
| | | CAAGGTCCACGTGCACTGCTCGCGCCGCAGGAAGCTTCAGG |
| | | CTTGGCCTGGTGGAGTTAGGGCTGCTCCACAACTGTGCGCAG |
| | | GGCATCCAGCAATTACAGTTGTGAAATAAGATATTTTGACTT |
| | | TTGGCTTCAAATTATTATTCATCGTAATTCCGTTTTCTTAAAT |
| | | GGCTCTCATTCATGGCGGAGCTCTTTGAGGTGAGAGTGTTTT |
| | | AATCATTTTATGGCCGGTACCTGACACATTGACTGGCATGTG |
| | | GTATAAGCTCAATAATCTTCTGTTAAATTAATGAATAAATGT |
| | | GCTCAGCTGCCAATCCACTTAGGCTCAAGAAAAACCAGAGG |
| | | TAAACAGAACCTTAAAAATGGACTTTTATTAATTATTTTCTA |
| | | TCATTTTGCTTAATTCTTTAAAGTAAACTCTTATTGACTTGGA |
| | | TCTAAATAGTTTGTGAATACAAAGTCTGAGGAAAAAAGTGT |
| | | TTGCTAAAAATAAAAACAACACTTGAATGATGTTTGTAAGG |
| | | CAGTTTTAATTTCTTAGAAAAGCTGAACAAATGGCACAATGC |
| | | AAAGAGCAGAAGTTTTGGAATAAATAGATTGAAGCCATTAA |
| | | ATTATTGGATAAAAATAGTTTCAGGTTGCTTTTGGCCTAGGT |
| | | TCTCCCCTCCCCCCATCACTATCCACTTCAGGAATAAACATT |
| | | CTGAAAGTTAATTTTACCCATATAGTGAGCACTTATTTCTAA |
| | | GTTGCCTTATCAAATACCATCTATGTTATGTCATTTAATCTCG |
| | | CAGTTGCCTGTGCATTAGAGATTAGCATCACCACTTTATATA |
| | | TCCTAATATTAGTACATGATAAACACTTTAAGTAATCAGCCC |
| | | ACAAGTACTCACCAAGACCTTAAGCCTCCCAAAGTACACAA |
| | | CATTCTTACGTTCCTCACTACACATCTGTAGAGTCAAAGGGA |
| | | CATAAAGCCTTGTTAAAGCCAGTTTTGACCAGAAGCAGCAA |
| | | TGGTCTCTTCCTGTTTGATCTCCATGTTAATGGGACAAAATG |
| | | ATACTTTCAAGGCATTGAAAATTTATGATTAATCAATCAAAT |
| | | TTATGATTAATCAATCCCTAGTCTGACTCCAGTGAGATTCAC |
| | | AAAACTTTCAGTTTACTTTATACTCCCTTGCCTTCTTTTGACT |
| | | CACATCATAGTGCCGGCAAGTACTTATATTTTTGCTATTTCA |
| | | GTTCTATTTCCATAAAATTTATTTTATCATCTTTTCTCATAAA |
| | | TTTGTGCCCTCTATTTTTACTCCCAATCTGTGTAAGATGAACA |
| | | AATCTTATAAGACCACATAGCTGACTGTGATTTCAGGTGGAC |
| | | TCCAGGAAGGAGAACCAAAGAAAACTTTCAGTCTTTTTAAG |
| | | ATGAACAAATCTTGTAAGTCCCCACATAGCTGACTGTGATTT |
| | | CAGGTGGACTCCAGGAAGGAGAACCAAAGAAAAGTTCAAGT |
| | | CCAAGCAGAAACCGTGATTCCTTCCGGATGATGGCTCATGA |
| | | GTGCCATTTAATTGGGGTGCCACCTGCTGTCCTCAGCAAATC |
| | | CCAGCTATATGTATATGTTCGCATTACAGGCTCATTAACCTA |
| | | GGCTGACCTCTGCAAGGATCTCAGAATATTTTCTACAGAGAA |
| | | CATACATGATAATATCTGATTTTAGGACAAAAAAGTAATTCT |
| | | CAATAGCAAGGGAATGGAGTAGGGTAGACAGCTAGTAATTA |
| | | AACTCACTTGTGTGTTAAAAATAAATTAAGGAAAAAAGAAA |
| | | ATGAGAGAACATATTACTAAATAAAGAAAGCATACATTAAA |
| | | TATTTACTATAGTTTTACACTAAGAGAATAAAGGAAATGCAA |
| | | TAAAGTGGCCTGAAAGGTAAAGGATGAGATGTGTAAAAGAG |
| | | GCGGGGAAAGATGTGTCATTTTTTTTTTACTATGAGCAGCAA |
| | | TCTGAGAAGATAAAGGAATCGAGTTATGGGCAGACATGATG |
| | | TTTGATCAGTGTTATTTGTTTTCAAGGCCTGCCTACTTTTTTT |
| | | TCAAATATTACAAACTTTTGAAGTCACATTCTTTTTGTTTTTT |
| | | GCTGTCTGTTACTAGATCGCACATTCTGTAAAGGCAGGGACC |
| | | ATGGTATGTTGTTTATCTTTGGATTCTCAGTGATTGTCATATT |
| | | TATGTTTGTTGAATGAATCTTAATCCAAGACTTGGGCTCCAG |
| | | GTATCTTTCCATTCTGGTTCCAAGGAGGGACCTTCCTCACAG |
| | | CAGGCGTGCTGTGTGGTCTCACATCTCACTCCTATATCTTTCC |
| | | CTGTCTGTTACTGCCCTCAGTGGAGCCCACAGTGACCATCTC |
| | | CCCATCCAGGACAGAGGCCCTCAACCACCACAACCTGCTGG |
| | | TCTGCTCAGTGACAGATTTCTATCCAGCCCAGATCAAAGTCC |
| | | GGTGGTTTCGGAATGACCAGGAGGAGACAACCGGAGTTGTG |
| | | TCCACCCCCCTTATTAGGAACGGTGACTGGACCTTCCAGATC |
| | | CTGGTGATGCTGGAAATGACTCCCCAGCATGGAGACGTCTA |
| | | CACCTGCCACGTGGAGCACCCCAGCCTCCAGAACCCCATCA |
| | | CCGTGGAGTGGCGTAAGGGGATATTGAGTTTCTGTTACTATG |
| | | GGCCCCACAAGACAAAGGGCAGAGCTCATTCTGACCCATCC |
| | | CTTCCCATCTCTTATCCCTGATGTCACTACTGAGCTGGGAAT |
| | | CACAGGAGACTAGAGCACTTCTTCCTCCATGGCAAGTGCATC |
| | | AGAAGAATCCTGATCTCATCACCTTTCCAGATGCTAGGGAAA |
| | | TTACTCTACATACTGTTTCTCTGGATCCCAGTCCTGATAGCTC |
| | | GGAGGGACTGATCATTAGGGCTGGTGACTGGGATCTTAGGG |
| | | TTTAAGGTTATGGATGAGCTCCTGAGGAGTGGAGATCTGCTT |
| | | CTCCACTCTCTCACCTACTCACTGTACCCAAGGACCTATTGG |
| | | CTGGCCTTTCCCCTCCCTTAGGGGTGGTCTGAATGGAGGACT |
| | | AGTTTCCTTTGACGCCTTCACCTCCTGAATCTCAGACTGGAC |
| | | TTCAGCTCCTCAGCAGGGATGCTATGGGGTGTGGGGACAAA |
| | | CACTGACACTCAGGCTCTGCTTCTTAGGGGCTCAGTCTGAAT |
| | | CTGCCCAGAGCAAGATGCTGAGTGGCATTGGAGGCTTCGTG |
| | | CTGGGGCTCATCTTCCTCGGGCTGGGCCTTATTATCCATCAC |
| | | AGGAGTCAGAAAGGTGAGGAACCCCAAGGGAAAAGGGGAA |
| | | GATGGGCTGTGACCCAGACCCTCTGTTCAGAGAGGTCCTGTC |
| | | TCTAGATGTGGCTCTTTCCTCCTGACCCCGAGAGGAAGAAAG |
| | | CTGAGCTGGAGGTGGGAGGAGACAGGACAAGATTGGAGGA |
| | | GGCATTGGAATCTGATTTTACTAGTTGAAAGGTAGCCCTGTC |
| | | ACACAGCTCACTGATAGAGCTTATTGAAGGACATCCTTACCT |
| | | TTCATCATTGTCTCATTGGCTCCTTTCCAAAAGCTTCCTCCAT |
| | | TAAGAGGGTCAGAGCCTTGGCCTCCTTGCCTTCTAGTGACAA |
| | | TTTCCTTTGTTTTAGGGGATTTTAAATTAGGGTGCTTAAGGCC |
| | | TTGAAGGACATGGGTGGGAAGAGAATATAACTCTAATTAAG |
| | | TCACATGTGTCATTTTCCATTGGGGTGAGAGAGTGGCTGTTT |
| | | GTGTAATGAGACCTTTCTCTGCATAACTTCCTTTTGTAAGAC |
| | | CTCAAGGGCCTCCACCAGCAGGTAATATTTCAGCCGTGATCC |
| | | AGTGTGGGGAGGGCACAGATGTAAGAGGGAAGAGCATGAG |
| | | CTGAGTGTACCTGACCACAGTGGTCTCCGTTCATGGTATATT |
| | | TGCTGCTATGAGGATCAAGACTTAGGGGCGAAGTTTGCCAG |
| | | TTTCTAGGAATCTCCAGAGGCTGTTCCCCAGAACCAAGCCTT |
| | | AACTTTGGTGGCATCTTCCTGTGAAATGTGAAGCCAGAACCA |
| | | CAGCTTAAATGTTAGACACTAGGATGATGCCCACTTTGTGCC |
| | | ACATGTTGGTGGCTACTGCCTGTAGGCATTTTCCAGTGACTG |
| | | AAAGAGGCTGCTAGTGGTAGGGATGAGGTATCATCCAATTT |
| | | CCTAAAAAGACCGAACCCTTCATATTCCCCAGAAGAGTAAC |
| | | AGCTGTTCCCCCACCTCCCTCACATCTGCATCAAGCTGAAGT |
| | | TCTGTGTCTTCATGAGCTGATTTCACCTTTGCACAGATCTTGG |
| | | GGGAGGTGATGACAATACACTCTGGACCTCAGCTTTGTCTGT |
| | | CTGAAGCTGCAGGGGGCCCCTGAGGGGTGGGGGAGATGGCA |
| | | GGCCCACCAGCGTACCCTGTGCTGATCATCCCTCTTCTCTCTT |
| | | CTCCAGGGCTCCTGCACTGACTCCTGAG |
| 26 | HLA- | TGTACCAGTTTAAGGCCATGTGCTACTTCACCAACGGGACGG |
| | DQB1 | AGCGCGTGCGTTATGTGACCAGATACATCTATAACCGAGAG |
| | | GAGTACGCACGCTTCGACAGCGACGTGGGGGTGTATCGGGC |
| | | GGTGACGCCGCTGGGGCCGCCTGACGCCGAGTACTGGAACA |
| | | GCCAGAAGGAAGTCCTGGAGAGGACCCGGGCGGAGTTGGAC |
| | | ACGGTGTGCAGACACAACTAC |
| 27 | HLA- | TCACTAATGTGCTTCAGGTATATCCCTGTCTAGAAGTCAGAT |
| | DRA | TGGGGTTAAAGAGTCTGTCCGTGATTGACTAACAGTCTTAAA |
| | | TACTTGATTTGTTGTTGTTGTTGTCCTGTTTGTTTAAGAACTT |
| | | TACTTCTTTATCCAATGAACGGAGTATCTTGTGTCCTGGACC |
| | | CTTTGCAAGAACCCTTCCCCTAGCAACAGATGCGTCATCTCA |
| | | AAATATTTTTCTGATTGGCCAAAGAGTAATTGATTTGCATTT |
| | | TAATGGTCAGACTCTATTACACCCCACATTCTCTTTTCTTTTA |
| | | TTCTTGTCTGTTCTGCCTCACTCCCGAGCTCTACTGACTCCCA |
| | | AAAGAGCGCCCAAGAAGAAAATGGCCATAAGTGGAGTCCCT |
| | | GTGCTAGGATTTTTCATCATAGCTGTGCTGATGAGCGCTCAG |
| | | GAATCATGGGCTATCAAAGGTAGGTGCTGAGGGAATGAAAT |
| | | CTGGGACGATAGACTACGAAGCATTGGAGAAAAGACCTATG |
| | | GACATTTGGAAGATAATGTGTGGAGTGAAAGAATAGTGTGA |
| | | CAGGTATTATGTGGTCTCGACAGAAAGTATAACAAATTGTG |
| | | GTTTGGTGGAGTTCTTCCCTCACCACAAACTGAAGTAAGTCA |
| | | AATTTGGTTTAGAGGGTCAAAACTGAGTTGTGTATTGATGAA |
| | | TAGCACGGTCCTGCTACAAGCCAAACTGGGGGTGGGGGTGG |
| | | GGGTGGGGGAGGAAGAATATTTTCTGGCAAGCATTAACAAG |
| | | TTATATTTCTGGGCTTTAATTATTCTTTCTGGAAAATTAGTAA |
| | | AATTAAAAACTAAAAACCACACATAGTTTTGCTAGAATTAA |
| | | ATGAAAAAAAAAGTTATTAGCCCTGTTCTTATCTGAATACAT |
| | | GATACAGTAGTTATTTTTTGGAGTGTAAATCCTGTCGGTATA |
| | | TATTGAGCACATATATTGTGTTGAAGATTACTAGAAGGAAA |
| | | AGTCATCAAAAAGCAACAATTTACCCCAGGAAAAGGGGAGG |
| | | GAAGGCATGCTGATATGAGTTGCCTCATGGGACAGTGATAG |
| | | CCATTCCCTGCCTTCCCATCTCCATGGTACAGCAGATCTTAT |
| | | ATCATGTTAACTTAGTAATATTTCCAAGAGAGTAGAAAAATA |
| | | AGTAAGGAAATGGGGAATCTGATATTATTGTCTCTCATCTCC |
| | | AGAGCAACATTGGTGCTGTTGTAAAGATGTACTGTAGAAAA |
| | | GTATTCTTCACCCAGCGTGACCCCCACAGAAGGTGTCAGGTA |
| | | GACTTGAAATAAGCAAAGTAATAACCCAGCTCCCATACCCA |
| | | TAGTGGCAATTGTAGATTTCTATTGCCCCAAAAGAGCCATAC |
| | | ATAGGGATACTTACCTAGAAAGACAGAGGATCTTCCCTTGGT |
| | | TTGTGAAGAGGCAGCTAGTATATTTGTGTGTGTTTGCATAGA |
| | | TGCAAACGGTAAATAAATTCCTAGGTTTATCAATACACAGTC |
| | | AAACATTAAAATCTCTCATCTTGGCTGGGCACGGTGGCTCAC |
| | | GCCTGTAATCCCAGCACTTTGGGAGGCCGAGGCGGGCGGAT |
| | | CACGAGGTCAAGAGATCGAGACCGTCCTGGGCAACATGGTG |
| | | AAACCCCGTCTCTACTAAAAATACAAAAAATTAGCTGGGTA |
| | | TGGTGGCACACGCCTGTAGTCCCAGCTACTCGGGAGGCTGA |
| | | GGCAGGAGGATTGCTTGAGCCCGGGAGGCGGAGGTTGCAGT |
| | | GAGCTGAGATGGTGCCACTGCACTCCAGCCTGGCGATAGAG |
| | | CAAGACTCCGTCTCAAACAACCAAACCAAAACAAAACAAAA |
| | | TATCTCACCTTATCTTTGAAGACTAAGGAAAAAAAAAATCTC |
| | | CCACTCATCGATACACTCCACAGAGGCAGCATACTCTCCCAG |
| | | TGTAGCTTTCTCTTTTCATGTTCATTATTCCCTTGGTGTTGGTT |
| | | ATTCTCAATGTCAATCGTAACAGAACATCTTCCATAATAACA |
| | | GTCCCAATTTAAGGAGCATTAAGATAAAAGGTGGAATTGCC |
| | | AAGGTCAATCCAGACGAGAACCTTCTCATAGAGGTAACCAC |
| | | CGTGTGGGTTTGGATGCTGGGAAGCAGGGGGACTATGACGC |
| | | TACAAGGTCTCAGTCTTAATTTTTGGAGTACTTCAGTCCCCA |
| | | GGTATATTTTCCATAGATTTGGCCCTTAAATAAAAAGAAGCT |
| | | TCTGACTCTAAAATGTAAACAGTGCTTGTTACAGTCTTGTTG |
| | | ATATATTAAGAAATTACTCACCTTATCTCATTTAATCTTAAA |
| | | AACAAACCCCTGACAGGATCAAAACCACAGCAGGACTACAT |
| | | AATAGGAAAACTATACATAAATAGGTAGAATAATCTGCTCA |
| | | GGATCACTAGGTAAGTTGCTGAATAAGAATTCAAGATGTTTT |
| | | TGATCCCAGAGTTTAAAACCCAACCTTTCAAACAGTGTTTCC |
| | | TTCTTCTTAGAGTACAATGTTCTGAGAAAGAGATCCTCTGGA |
| | | ATTCTGGCCTAAGTGTATTTAATGCCCGGGTAAAGAAAGTGA |
| | | GAGAACATTTCTCTTTAGGGGCTGCTGCTGGATTTCTAAAAA |
| | | GAAAATAATTTCTCAGCTAGTAACATGGAGCCAAACAACAG |
| | | CTTCACAAGACTCTGGGTTCTTTAGCCCTCATCTCCTTCAATC |
| | | CACCCTCTTTATAACCAGTCCTTCTTGTTTTTCCCCTCCCAGC |
| | | TTTGTTCAGCAGCATGCCCTTCACCCAGACCTTGTCTTGTCAC |
| | | TCATCCCTACTCGCCATCATTCTTTCATTCCTCTTGGCCCAAT |
| | | CTCTCTCCACCACTTCCTGCCTACATGTATGTAGGTTATTCAT |
| | | TTCCCTCTCTTGATTCCCCCCACCCAACTCTCTTTCTCCATTT |
| | | CTTGCCTTTCAGAAGAACATGTGATCATCCAGGCCGAGTTCT |
| | | ATCTGAATCCTGACCAATCAGGCGAGTTTATGTTTGACTTTG |
| | | ATGGTGATGAGATTTTCCATGTGGATATGGCAAAGAAGGAG |
| | | ACGGTCTGGCGGCTTGAAGAATTTGGACGATTTGCCAGCTTT |
| | | GAGGCTCAAGGTGCATTGGCCAACATAGCTGTGGACAAAGC |
| | | CAACCTGGAAATCATGACAAAGCGCTCCAACTATACTCCGA |
| | | TCACCAATGGTACCTCCCTCTCTGCTGCACTCCTGGACATGG |
| | | GAATCCATAGTTTGAAAGTAGTTGCTTCAGCTCTTTGTGTTA |
| | | GATTATTGTAACTGATTTTCCCTCCAAGGGCCTAACCTTGCC |
| | | ATTAACAAGCCCCAAATTCTCATGCCAGAGGTCTGAGAACTT |
| | | TATGGGTTTGATCCTATCTTGTTGTGCTCAAGTCTTGTCTCTG |
| | | TCATCCATGGTCTCCTACGAAGTCATTGCCCTAAGTTCATGC |
| | | TAGGGGAGCCAGAAGGGAAGTCCTTGGATATCTTATACCTC |
| | | AATATTGGCTCAATTTCTTGGGGAGGGGGTGCTGTCAGAGAT |
| | | TGTTATCTGAGGATGTGACATAGATTTCTCAGGGCACAATTT |
| | | CAACTACTTTTTCAGCTTTAGGGTTTTTAGATACGTTTGTACC |
| | | ACAATTGAGCATGGGAGGGAGAGGGGTGAGCCTAAGCAGTG |
| | | ATGGCTGATTTCTGTCACGTCTGTCATGTGTCCCCCAGTACCT |
| | | CCAGAGGTAACTGTGCTCACGAACAGCCCTGTGGAACTGAG |
| | | AGAGCCCAACGTCCTCATCTGTTTCATCGACAAGTTCACCCC |
| | | ACCAGTGGTCAATGTCACGTGGCTTCGAAATGGAAAACCTG |
| | | TCACCACAGGAGTGTCAGAGACAGTCTTCCTGCCCAGGGAA |
| | | GACCACCTTTTCCGCAAGTTCCACTATCTCCCCTTCCTGCCCT |
| | | CAACTGAGGACGTTTACGACTGCAGGGTGGAGCACTGGGGC |
| | | TTGGATGAGCCTCTTCTCAAGCACTGGGGTATGGACCAACAC |
| | | TCAATCTCCTTTATTTCAAGGTTTCCTCCTATGATGCTTGTGT |
| | | GAAACTCGGTGTTCTAACTGTTTCATAATATCTGCTACAATT |
| | | AATATAACTGTCTTCTCCTACTATCCAGCTTCCTCCTTTTTTT |
| | | AATCTGTAATTCTCTCAATACATCATTCTGTCTTCCTCTTCTT |
| | | TAATCTATGAATAACTTTTCTCTTTATTAAGAACCCTACATTT |
| | | GATTCTGAGTGTTACTTCTTCCCACACTCATTACCATGTACTC |
| | | TGCCTTATCTCCCCCCAGAGTTTGATGCTCCAAGCCCTCTCCC |
| | | AGAGACTACAGAGAACGTGGTGTGTGCCCTGGGCCTGACTG |
| | | TGGGTCTGGTGGGCATCATTATTGGGACCATCTTCATCATCA |
| | | AGGGAGTGCGCAAAAGCAATGCAGCAGAACGCAGGGGGCC |
| | | TCTGTAAGGCACATGGAGGTGAGTTAGGTGTGGTCAGAGGA |
| | | AGACATATATGGAGATATCTGAGGGAGGAAAACAGGGTGGG |
| | | GAAAGGAAATGTAATGCATTTAAGAGACAAGGTAGGAACAG |
| | | ATGTGGCTCTTGATTTCTCTTTGCTAGAATGAATCAGACATT |
| | | GGTATCATCTGGTATCCCAAAGCTTCAGGGTCTGTCATCCCT |
| | | TTCTATAGACGGGCACCTTGATCACGGCTCCAGTCTTAGAAA |
| | | TCATCTCCAGTACCTAAAACCATTGTTTCACATTAGAATACT |
| | | GAGTCTAGGGATCTAGAAAATACATTAGAATATGGAGTCTA |
| | | GGGATCTAGAAAATACTGAGTCTAGGGATCTAGAAAAATAA |
| | | GCCTCAAGATTTGGGCACATCCTAGCTTGTATTTCCTGGGGC |
| | | AGGTCATCAGTTCAGAAGCATTTCCAGATCCTGGCTCCTTTC |
| | | AGGTTAGGGTCAATTCATTGCATGAAATGGGAATCTCTTAGA |
| | | GGCCAATGCCTGCTTTTGCTTCTTTAGTCTCAAATGTAGTATG |
| | | AGAAACTCTAAAAAAAGGTAAAGCATGGTTGCTTATTATGTT |
| | | CAGTTGGAGAGTAGGAACTAACTGTATACAGTTAGTTCATGT |
| | | TGGAAAGGTTAGATGAACATTGAAAGAATTTTGCAAAGTCA |
| | | AAGGATTAAGAGAGAAGAGGAAGGAATCTGAAGCAAGGAG |
| | | CTCAAAACGGATCTTAAATTCCTTGGTAACTATGTGTGTCTT |
| | | GCTATAGGTGATGGTGTTTCTTAGAGAGAAGATCACTGAAG |
| | | AAACTTCTGCTTTAATGACTTTACAAAGCTGGCAATATTACA |
| | | ATCCTTGACCTCAGTGAAAGCAGTCATCTTCAGCGTTTTCCA |
| | | GCCCTATAGCCACCCCAAGTGTGGTTATGCCTCCTCGATTGC |
| | | TCCGTACTCTAACATCTAGCTGGCTTCCCTGTCTATTGCCTTT |
| | | TCCTGTATCTATTTTCCTCTATTTCCTATCATTTTATTATCACC |
| | | ATGCAATGCCTCTGGAATAAAACATACAGGAGTCTGTCTCTG |
| | | CTATGGAATGCCCCATGGGGCATCTCTTGTGTACTTATTGTTT |
| | | AAGGTTTCCTCAAACTGTGATTTTTCTGAACACAATAAACTA |
| | | TTTTGATGATCTTGGGTGGAATTTTTGGTGTTTAAGCCAGTTC |
| | | TTTGGGTGGCGGTGGGGGGTGGGGAGTCGGTCCTGGGGAAT |
| | | ATATGTGATCCTTTCCCGGTAAAATATCTGAATGTTGAATTT |
| | | ATCTTATAAATTCTAGAATTCATCAGACATATCCCGGTTCAT |
| | | TTGGGCTTGGTCTCATTTTGTGCATCTGCAGGCAACCCTCTTG |
| | | TTGTGGTCTAGTCCTCATCAGGAAAACCTAAAGTGGGGTTGG |
| | | TTTGTTGGGAGATCTCTA |
| 28 | HLA- | TACGTCTGAGTGTCATTTCTTCAATGGGACGGAGCGGGTGCG |
| | DRB1 | GTTCCTGGACAGATACTTCCATAACCAGGAGGAGTTCGTGCG |
| | | CTTCGACAGCGACGTGGGGGAGTACCGGGCGGTGACGGAGC |
| | | TGGGGCGGCCTGCTGCGGAGCACTGGAACAGCCAGAAGGAC |
| | | CTCCTGGAGCGGAGGCGGGCCGAGGTGGACACCTATTGCAG |
| | | ACACAACTACGGGGTTGTGGAGAGCTTCACAGTGCAGCGGC |
| | | GAGGTGAGCGCGGCGCGGGGCGGGGCCTGAGTCCCTGTGAG |
| | | CTGGGAATCTGAGTGTGTGTGTGTGTGTGTGTGTGTGTGTGT |
| | | GTGTGTGTGTGTGTGTGTGTGTGTGTGAGAGAGAGAGAGAG |
| | | AGAGAGAGACAGAGAGAGAGAGAGCGCCATCTGTGAGCAT |
| | | TTAGAATCCTCTCTATCCTGAGCAAGGAGTTCTGAGGGCACA |
| | | GGTGTGTGTGTAGAGTGTGGATTTGTCTGTGTCTGTGAGGCT |
| | | GTTGTGGGAGGGGAGGCAGGAGGGGGCTGCTTCTTATTCTT |
| | | GGAGGACTCTGTGGGGAGGTGACAAGGGAGGTGGGTGCGG |
| | | GCGGCTGGAGAGAGAGGTGACCTTGATTGTCCTGGGTCCTTA |
| | | GAGATGCAGGGAAGGGAAATGTAAGGGGTGTGTGGTTGGGG |
| | | TGAAGGTTTAGGGGAGGAGAGCTGAGGGGTAAGGAAGGTTT |
| | | GGGATAATGTGAGGAGGCCGGTTCCAGACTGTCCCTGGCAC |
| | | ACACCCTTCATGTAATCTCTGAAATAAAAGTGTGTGCTGTTT |
| | | GTTTGTAAAAGCATTAGATTAATTTCTAGGGGAATTGAGGAG |
| | | ACCTCTGAGGCATCTCTGAAGCTTCTTTAGGTCTAAATTTCTT |
| | | GCTAGTTTTTTGTTTTTTATTGTGTATATTTTTACATAGTAGA |
| | | AATGACTGTGAAACTAACTTTTTGAATTAAAGTTTTAACACA |
| | | GTTACTATTTTATTATAATGCTAATAGTTTTCTAGTAGTTACA |
| | | TATTATTCTTTTATATATAATAGTTGTGACACAACTTACCTCA |
| | | CTTTCCCCTTTGTTGACCTTTATTATGACATTCACCAAAAGTT |
| | | GAAAATGTATGTTTCTGGTTAATTTTTAATTTATATTTTTTTC |
| | | ATTTATAATTCTTTTGAATTATTTTGACCTATTTATTGGCCAG |
| | | TTTTAATAACTGCTGTAAGAATTCCCTATTGTATTTGGTAGG |
| | | GAATGGACAATGATCTACTGCCTAATATCTCGAGGGCTTAGT |
| | | ATTTTTCTCAGTGACTTTGTGGGTTCTTTGTACTGTGAGATTA |
| | | TTAACACTTTATTGATATTTGATTCAGCATTTGCTCCAGTTTG |
| | | TGGTTTGTATGTTGATTTTGAAAATTCTTTTCCATGTTAAGAA |
| | | TTTGAACATTTTTATATAATAAAATATGTTGCAAAATTTTTAT |
| | | TAATGATTTACAATCCATCTTAAATCTGCCATTTTGTGGTATT |
| | | GTTGTCTCCAGGTTTCTCCTTACTTCTAAAAAAAATTGCATTT |
| | | ATTGAGAGTCTGCTAGTGTTAGGGATTTTCCTGGGCATAAGC |
| | | ACCCCAAGTGACGAGTCCCAGACACTGCCTTAATCCAAATGT |
| | | GATTCTGGAAAGAAAAATCATTTTACAATGATAGGCCTAAT |
| | | AATAATTAAGCTTGTGTTGCATGGGAGATGCATTGATCAGCT |
| | | AAATGTAAATATAAGAACTTTCAAAACTAAAATGACGTTCCT |
| | | TAATCCTTCTCTCTGCTTTATGAATCATGCTTTTCTGGGAAAG |
| | | TAAAAATTTGGAGAATCATTTCTGTCTGTCCCACCTTCCCAG |
| | | GGGCAGAACCATTTCTGTAGTGTTCTAAGGTGTGAGTGCATG |
| | | GCAGTAGTATTCCTAAAAATTCATATTCGGTTTCGTCATGTA |
| | | CCCAACTCTGTCCCGTTATCTATCCACATTGTTTTAAATCATA |
| | | TATTTCTGTCAAGGTGTACAAGGATGATAAATAGGTGCCAA |
| | | GTGGAGCACCCAAGTGTGATGAGCCCCCTCACAGTGGAATG |
| | | GAGTGTGAAGCTTTATGACCTCATAAATTGAAGGTTATCTTC |
| | | AGTCATTGTTTTATATATTTTACATGCATTAATCCTCATATAA |
| | | TCCCAAGAGGTAAATTAGTATAATTATCCTTCATTATAGGTG |
| | | ACAAAGTTGAGACACAGAAGAATCAAACTCTTAAGGCAGAC |
| | | CTTGGATTTGAACCAGGCAACCTGGCTCAGATATCCGTTTTA |
| | | ATTACTACACTCTGTACTTTCAAAGATTTGTAAACACTTTGA |
| | | CAATGCATGACAATTTCAAGCTATGAAGAAACAAACACAAT |
| | | TTTTCACAATATCTCTCAAATCTAATGGGTCCTCACTATCAA |
| | | GATTAAGTTCCAGGCTGATGACACTGTAAGGCCACATGGCC |
| | | AGCTGTGCTGGAGGCCTGGTCAAGGTCAGAGCCTGGGTTTG |
| | | CAGAGAAGCAGACAAACAGCCAAACAAGGAGACTTACTCTG |
| | | TCTTCATGACTCATTCCCTCTACCTTTTTTCTCCTAGTCCATC |
| | | CTAAGGTGACTGTGTATCCTTCAAAGACCCAGCCCCTGCAGC |
| | | ACCACAACCTGCTGGTCTGTTCTGTGAGTGGTTTCTATCCAG |
| | | GCAGCATTGAAGTCAGGTGGTTCCGGAATGGCCAGGAAGAG |
| | | AAGACTGGGGTGGTGTCCACAGGCCTGATCCACAATGGAGA |
| | | CTGGACCTTCCAGACCCTGGTGATGCTGGAAACAGTTCCTCG |
| | | GAGTGGAGAGGTTTACACCTGCCAAGTGGAGCACCCAAGCG |
| | | TGACAAGCCCTCTCACAGTGGAATGGAGTGAGCAGCTTTCTG |
| | | ACTTCATAAATTTCTCACCCACCAAGAAGGGGACTGTGCTCA |
| | | TCCCTGAGTGTCAGGTTTCTCCTCTCCGACATCCTATTTTCAT |
| | | TTGCTCCATGTTCTCATCTCCATCAGCACAGGTCACTGGGGG |
| | | TAGCCCTGTAGGTGTTTCTAGAAACACCTGTACCTCCTGGAG |
| | | AAGCAGTCTCGCCTGCCAGGCAGGAGAGGCTGTCCCTCTTTT |
| | | GAACCTCCCCATGATGTCACAGGTCAGGGTCACCCACCCTCC |
| | | CCGGGCTCCAGGCACTGCCTCTGGGTCTGAGACTGAGTTTCT |
| | | GGTGCTGTTGATCTGAGTTATTTGTTGTGATCTGGGAAGAGG |
| | | AGAAGTGTAGGGGCCTTCCTGACATGAGGGGAGTCCAATCT |
| | | CAGCTCTGCCTTTTATTAGCTCTGTCACTCTAGACAAACTACT |
| | | TAGCCTCATTGAGTCTCAGGCTTTCTGTGGATCAGATGTTGA |
| | | ACTCTTGCCTTACATCAAGGCTGTAATATTTGAATGAGTTTG |
| | | ATGTCTGAACCTTGTAACTGTTCAGTGTGATTTGAAATCCTTT |
| | | TTTTCTCCAGAAATGGCTAGTTATTTTAGTTCTTGTGGGGCA |
| | | GACTTCTTCCCCATTTTCAAAGCTCTGAATCTTAGAGTCTCA |
| | | ATTAAAGAGGTTCAATTTGGAATAAGCATCACTAAACCTGG |
| | | CTTCCTCTCTCAGGAGCACGGTCTGAATCTGCACAGAGCAAG |
| | | ATGCTGAGTGGAGTCGGGGGCTTTGTGCTGGGCCTGCTCTTC |
| | | CTTGGGGCCGGGCTGTTCATCTACTTCAGGAATCAGAAAGGT |
| | | GAGGAGCCTTTGGTAGCTGGCTCTCTCCATAGGCTTTTCTGG |
| | | AGGAGGAACTATGGCTTTGCTGAGGTTAGTTCTCAGTATATG |
| | | AGTGGCCCTGAATAAAGCCTTTCTTTCCCCAAACGGCTCTAA |
| | | TGTCCTGCTAATCCAGAAATCATCAGTGCATGGTTACTATGT |
| | | GAAAGCATAATAGCTTGTGGCCTGCAGAGACAAGAGGAAGG |
| | | TTAACAAGTAGGGGTCCTTTGGTTTGAGATCTTGGAGCAGAT |
| | | TAAGGAAGAGCCACTAAGACTAATGGAATTACACTGGATCC |
| | | TGTGACAGACACTTCACCCTTCATGGGTCACATGGTCTGTTT |
| | | CTGCTCCTCTCTGCCCTGGCTGGTGTGGGTTGTAGTGACAGA |
| | | GAACTCTCCGGTGGGAGATCTGGGGCTGGGACATTGTGTTG |
| | | GAAGACAGATTTGCTTCCATAAATTTTAAGTGTATATATTTT |
| | | CCTCTTTTTCCCAGGACACTCTGGACTTCAGCCAAGAGGTAA |
| | | TACCTTTTAATCCTCTTTTAGAAACAGATACGGTTTCCCTAGT |
| | | GAGAGGTGAAGCCAGCTGGACTTCTGGGTCGGGTAGGGACT |
| | | TGCAGAACTTTTCTGTCTTAGGAGAGGTTTCTAAATGCACCA |
| | | ATCAGTGCTCTGTAAAAACACACCCATTGGCACTCTGTGGCT |
| | | AGATAGATGTTTGTAAAATGGACTAATCAGCACTCTGTAAA |
| | | ATGGAGCAATCCACACTCTGTAAAATGGGCCAATCAATGCT |
| | | CTTTAAAATGGACCAATCAGCAGGACATGGGCGGGGACAAA |
| | | TAAGGGAATACAAGCTGGCCACCCCAGCCAGCAGCAGCAAC |
| | | CCGCTCAGGTCGCCTTCCATGCTGTGGAAGCTTTGTTCTTTTG |
| | | CTCTTCACAATAAATCTTGCTGTTGCTCACTCTTCGGGTCTGT |
| | | GCCACCTTTAAGAGCTGTAACACTCACTGTGAAGATTCGCGG |
| | | CTTCATTCTTGAAGTCAGCAAAACCACGAACCCACCGGAAG |
| | | GAACAAACTCTGGACACACTAGAATTGATGGTAGAGGTGAT |
| | | AAGGCATGAGACAGAAATAATAGGAAAGACTTTGGATCCAA |
| | | ATTTCTGATCAGGCAATTTACACCAAAACTCCTCCTCTCCAC |
| | | TTAGAAAAGGCCTGTGCTCTGTGGGACTATTGGCTCTGGGAG |
| | | ACTCAGGAACTTGTTTTTCTTCTTCCTGCAGTGCTCTCATCTG |
| | | AGTCCCTGAAAGAGAGGAAAAGAAACTGTTAGTAGAGTCAG |
| | | GTTGAAAACAACACTCTCCTCTGTCTTTTGCAGGATTCCTGA |
| | | GCTGAAGTGCAGATGACACATTCAAAGAAGAACTTTCTGCC |
| | | CCAGCTTTGCAGGATGAAAAGCTTTTCCTCCTGGCTGTTATT |
| | | CTTCCACAAGAGAGGGCTTTCTCAGGACCTGGTTGCTACTGG |
| | | TTCAGCAACTGCAGAAAATGTCCTCCCTTGTGGCTTCCTCAG |
| | | CTCCTGCCCTTGGCCTGAAGTCCCAGCATTGGTGGCAGCGCC |
| | | TCATCTTCAACTTTTGTGCTCCCCTTTGCCTAA |
| 29 | HLA- | GAGTGTCATTTCTTCAATGGGACGGAGCGGGTGCGGTTCCTG |
| | DRB1 | GACAGATACTTCTATAACCAGGAGGAGTCCGTGCGCTTCGA |
| | | CAGCGACGTGGGGGAGTACCGGGCGGTGACGGAGCTGGGGC |
| | | GGCCTGACGCTGAGTACTGGAACAGCCAGAAGGACCTCCTG |
| | | GAAGACAGGCGCGCCGCGGTGGACACCTACTGCAGACACAA |
| | | CTACGGGGTTGGTGAGAGCTTCACAGTGCAGCGGCGAGGTG |
| | | AGCATGGTGGGGGGCGGGGCCTGGGTCCTTGTGAGCTGGGA |
| | | ATCTGAGTGTGTGTGTGTGTGTGTGTGTGTGTGTGTGTGTGT |
| | | GAGAGAGAGAGAGAGAGGAAGAGAGAGAGAGAGCGCCATC |
| | | TGTGAGCATTTAGAATCCTCTCAGTCCGGAGCAAGCAGTTCT |
| | | GAGAGCACAGGTGTGTGTGTAAAGTGTGGATTTGTCTGTGTC |
| | | TGTGTGTCTGTTGTGGGAGGGGAGGCAGGAGGGGGCTGTTT |
| | | CTTATCCTTGGAGACCTCTGTGGGGAGGTGACAAGGGAGGT |
| | | GGGTGCTGGGGGCTGGAGAGAGAGGAGACCTTGATTATCTC |
| | | GGTCCTTAGAGATGCAGGGAAGGGAAATGTAAGGGGTGTGT |
| | | GGTTGGGGTGAAGGTTTAGGGGAGGACTGCTGAGGGGTAAG |
| | | GCAGGTTTGGGATAATGTGAGGAGGCCAGTTCCAGACTCTC |
| | | CCTGGCATACACCCTTCGTGTAATCTCTGAATTAAAAGTGTG |
| | | TGCTGTTTGTTTGTAAAAGCAATAGATTAATTTCTAGAGGAA |
| | | CTGAGTAGACCTCTGAGGCACCTCTGAAGCTTCATTAGATCT |
| | | AAATTTCTTCCTAGTTTTTTGTTTTTTTCAGTGTGTATATTTTT |
| | | ACATAGTAGAAATGACTGTGAAACTAACTTTTTGAATTAAAG |
| | | TTTTGACACGGTTACTATTTTATTATAATGCTGGTAGTTTTCT |
| | | AGTAGTTACATATTATTCTTTTATATATAATATTTGTGACACA |
| | | ACTCACCTCACTTTCCCGTTTGTTGACCTTTATTATGACATTC |
| | | ACCAGAAGTTGAAATTGTGTGTTTCTGGTTAATTTTTAATTTA |
| | | TATTTTTTATTTGTAATTCCTTTGAATTATTTTGCCCTATTTAT |
| | | TGGCCGATTATAATTATTGCTCTAAGAATTCCCTATTGTATTT |
| | | GGTAGGTAATGGACAATGATCTACGGTCTAATATCTTGAGG |
| | | GCTTAGTATTTTTCTCAGTGACTTTCTGCGTTCTTTGTACTAT |
| | | AAGATTATTAACACTTTATTGATATTTGATCCAGCATTTGCTC |
| | | CAGTTTGTGGTTTGTATGTGGATTTTGAAAATTCTTTTCCATG |
| | | TTAAGAATTTGAACATTTTTATTTAATAAAATATATTGCAAC |
| | | ATTTTTATTAATGATCTACAATCCATCTGAAATCTGCCATTTT |
| | | GTGGCATTGTTGTCTCCAGGTTTCTCCTTACTTCTAAAAAAAT |
| | | AGTTGTATTTATTGAGAGTATGCTAGTGTCGGGGATTTTCCT |
| | | GGGCATAAGCACCCCAAGTAACGAGTCCCAGACACTGCCTT |
| | | AATCCAAATGTGATTCTGGAAAGAAAAATCATTTTACAATG |
| | | ATAGGCCTAATAATAATTATGCTTGTGTTGCACGGGAGATGC |
| | | ATTGATCAGCTAAATGTAAATATAAGAACTTTCAAAACTTAA |
| | | ATGACGTTCCCTAATCCTTCTCTCTGCTTCAGGACTCATGCTT |
| | | TTCTAGGAAAGTAAAAATTTGGAGAATCATTTCTGTCTGTCC |
| | | CACCTTCCCAGGGGCAGAACAATTTCTGTTGTGTTCTAAGGT |
| | | GTGAGTGCATGGGAGTAGTATTCCTAAAATTCATACTTGGTT |
| | | TCCTCATGTACCCAACTCTGTCCCTTTATCTATGCATATTGCT |
| | | TTAAATCATATTTTTCTGTCAAGGTGTACAAGGATGATAAAT |
| | | AGGTGCCAAGTGGAGCACTCAAGTGTGATGAGTGCCCTCAC |
| | | AGTGGAATGGAGTGAGAAGCTTTCTGACCTCATAAACTGAA |
| | | GGCTATCTTCAGTCATTGTTTTATATATTTTACGTGCATTAAT |
| | | CCTCATATAACCCCAAGAGGTAAATTAGTATAATTATCCTTC |
| | | ATTGTAGGTGACAAAGTTGAGACACAGAAGAATCAAAAAAC |
| | | TCTTCCAGGATCAACCAGTAAAAGGCAGACCTTGGATTCGA |
| | | ACCAGGCAACCTGGCTCAAATATCAGTTTTAATTACTACACT |
| | | CTGTACTTTCCAAGATTTGTAAACAGTTTGACAATGCATGCC |
| | | GATTTAAAACTATGAAGAAACAAACACAATTTTTCACAACA |
| | | CCTCTCAAATCTAATGGGTCCTCACTGTCAAGATTAAATTCC |
| | | AGGCTGATGACACTGTAAGGTCACATGGCCAGCTGTGCTAT |
| | | AGGCCTGGTCAAGGCCAGAGCCTGGGTTTGCAGAGAAGCAG |
| | | ACACACAGCCAAACCAGGAGACTTACTCTGTCTTCCTGACTC |
| | | ATTCCCTCTACGTTGTTTTTCTCCTAGTCCAACCTAAGGTGAC |
| | | TGTATATCCTTCAAAGACCCAGCCCCTGCAGCACCACAACCT |
| | | CCTGGTCTGCTCTGTGAGTGGTTTCTATCCAGGCAGCATTGA |
| | | AGTCAGGTGGTTCCTGAACGGCCAGGAAGAGAAGGCTGGGA |
| | | TGGTGTCCACAGGCCTGATCCAGAATGGAGACTGGACCTTCC |
| | | AGACCCTGGTGATGCTGGAAACAGTTCCTCGAAGTGGAGAG |
| | | GTTTACACCTGCCAAGTGGAGCACCCAAGCGTGACAAGCCC |
| | | TCTCACAGTGGAATGGAGTGAGCAGCTTTCTGACTTCCTAAA |
| | | TTTCTCACCCACTAAGAAGGGGACCGTGCTAATCCCTGAGTG |
| | | TCAGGTTCCTCCTCTCCCACATCCTATTTTAATTTGCTCCATG |
| | | TTCTCATCTCCATCAGCACAGGTCACTGGGGGTAGCCCTGTA |
| | | GGTGTTTCTAGAAACACCTGTACCTCCTGGAGAAGCAGTCTC |
| | | GCCTGCCAGGCGGGAGAGACTGTCCCTCTTTTGAACCTCCCC |
| | | ATGATTTTGCAGGTCAGGGTCACCCACTCTCCCAGGCTCCAG |
| | | GCCCTGCCTCTGGGTCTGAGACTGAGTTTCTGGTGCTGTTGC |
| | | TCTGAGTTATTTTTTGTGATCTGGGAAGAGGAGAAGTGTAGG |
| | | GGCCTACCTGACATGAGGGGAGTCCAATCTCAGCCCTGCTTT |
| | | TTATTAGCTTTGTCACTCTAGACAAACTACTTATCCTCATTGA |
| | | GTCTCAGGCTTTCTGTGGATCAGATGTTGAACTTGTGCCTTA |
| | | CATCAAGGCTGTAATATTTGAATGAGTTTGATGTCTGAACAT |
| | | CGTAACTGTTCAGTGTAATTTGAAATCCTTTTTTTCTCCTGAA |
| | | ATGGCTAGTTATTTTAGTTCTTGTGAGGCAGCTTTCTGCCCCA |
| | | TTTTCAAAGCTCTGAATCTTAGAGTCTCAAGTAAAAAGGTTC |
| | | AATTTGGAATAAACATCACTAAACCTGGCTTCCTCTCTCAGG |
| | | AGCACGGTCTGAATCTGCACAGAGCAAGATGCTGAGTGGAG |
| | | TCGGGGGCTTTGTGCTGGGCCTGCTCTTCCTTGGGGCCGGGC |
| | | TGTTCATCTACTTCAGGAATCAGAAAGGTGAGGAGCCTTTGG |
| | | GAGCTGGCTCTCTCCATAAGCTTTTCTGGAGGAGGAACTATG |
| | | GCTTTGCTGAGGTTAGTTCTCAGTATATGAGTGGCCCTGAAT |
| | | AAAGCCTTTCTTTCCCCAAACGGCTCTAATGTCCTGCTAATC |
| | | CAGAAATCATCAGTGCATGGTTACTATGTGAAAGCATAATA |
| | | GCTTGTGGCCTGCAGAGACGAGAGGAAGGTTAACAAGTAGG |
| | | GGTCCTCTGGTTTGAGATCCTGGAGCAAATTAAGGAAGAGC |
| | | CACTAAGGCTAATGGAATTACACTGGATCCTGTGACAGACA |
| | | CTTCAGGCTTCATGGGTCACATGGTCTGTTTCTGCTCCTTTCT |
| | | GCCCTGGTTGGTGTGGGTTGTGGTGTTAGAGAAATCTCAGGT |
| | | GGGAGATCTGGGGCTGGGACATTGTGTTGGAGGACAGATTT |
| | | GCTTCAATAACTTTTAAGTGTATATCTTTTCCTCTTTTTCCCA |
| | | GGACACTCTGGACTTCAGCCAACAGGTAATACCTTTTAATCC |
| | | TCTTTTAGAAACAGACACAGTTTCCCTAGTGAGAGGTGAAGC |
| | | CAGCTGGACTTCTGGGTGGGGTGGGGACTTGGAGAACTTTTC |
| | | TTACAAGAGGTTTCTTTTTTTTTTTTTTTTTGAGACGGAGTCT |
| | | CGCTCTGTCGCCCAGGCTGGAGTGCAGTGGCGGGATCTCGG |
| | | CTCACTGCAAGCTCCGCCTCCCGGGTTCACGCCATTCTCCTG |
| | | CCTCAGCCTCCCAAGTAGCTGGGACTACAGGCGCCCGCCACT |
| | | ACGCCCGGCTAATTTTTTGTATTTTTAGTAGAGACGGGGTTT |
| | | CACCGTTTTAGCCAGGATGGTCTCGATCTCCTGACCTCGTGA |
| | | TCCGCCCGCCTCGGCCTCCCAAAGTGCTGGGATTACAGGTGT |
| | | GAGCCACCGCGCCCGGCCTACAAGAGGTTTCTAAATGCACC |
| | | AATCAGTGCTTTGTAAAAACACACCAATAGGTTCTCTGTGGC |
| | | TAGCTGGATGTTTGTAAAATGGACCAATTCGCACTCTGTAAA |
| | | ATAGACCAATCAGCACTCTTTAAAATGGACCAATCAGCACTT |
| | | TTAAAATGGGCCAATCACCACTCTTTAAAATGGACCAATCAG |
| | | CACTCTTTAAAATGGACCAATCTGCAGGACATGGGCAGGGA |
| | | CAAATACAGGAATAAAAGCTGGCCACCCCAACCAGCAGTGG |
| | | CAACCCACTCAGGTCCTCTTCCCTGCTGTGGAAGTTTTGTTCT |
| | | TTTGATCTTCACAATAAATCTTGCTGCTGCCCACTCTTTGGGT |
| | | CCCTGCCGCCTTTAAGAGCTGTAACACTCACTGTGAAGGTCT |
| | | GCAGGAAGTCAGCGAGACCACAAACCCACCGGAAGGAACA |
| | | AACTCAGGACACACCAGAATGATGGTAGAGGTGATAAGGCA |
| | | TGAGACAGAAATAATAGGAAAGACTTTGGATCCAAATTTCT |
| | | GATCAGGCAATTTACACCAAAATTCCTCCTCTCCACTTAGAA |
| | | AAGGCCTGTGCTCTGCGGGACTATTGGCTCAGGGGAGACTC |
| | | AGGAACTTCTTTTTCTTCTTCCTGCAGTGCTCTCATCTGAGCC |
| | | CTTGAAAGAGGGGAAAAGAAACTGTTAGTAGAGCCAGGTTG |
| | | AAAACAACACTCTCCTCTGTCTTTTGCAGGATTCCTGAGCTG |
| | | AAATGCAGATGACCACATTCAAGGAAGAACTTTCTGCCCCG |
| | | GCTTTGCAGGATGAAAAGCTTTCCTGCTTGGCAGTTATTCTT |
| | | CCACAAGAGAGGGCTTTCTCAGGACCTGGTTGCTACTGGTTC |
| | | GGCAACTGCAGAAAATGTCCTCCCTTGTGGCTTCCTCAGCTC |
| | | CTGCCCTTGGCCTGAAGTCCCAGCATTGATGGCAGCGCCTCA |
| | | TCTTCAACTTTTGTGCTCCCCTTTGCCTAA |
| 30 | HLA- | TAAGTCTGAGTGTCATTTCTTCAATGGGACGGAGCGGGTGCG |
| | DRB3 | GTTCCTGGAGAGATACTTCCATAACCAGGAGGAGTTCGTGC |
| | | GCTTCGACAGCGACGTGGGGGAGTACCGGGCGGTGACGGAG |
| | | CTGGGGCGGCCTGTCGCCGAGTCCTGGAACAGCCAGAAGGA |
| | | CCTCCTGGAGCAGAAGCGGGGCCAGGTGGACAATTACTGCA |
| | | GACACAACTACGGGGTTGTGGAGAGCTTCACAGTGCAGCGG |
| | | CGAGGTGAGCATGTCAGGGGCGGGGCCTGGGTCCCTGTGAG |
| | | CTGGAAATCTGAGAGTGTGTGTGTGTGTATGTGTGTATGTGT |
| | | GTGTGTGAGAGAGAGAGACAGAGAGGAAGAGAGAGACAGA |
| | | GAGAGGGAGCGCGCCATCTGTGAGCATTTAGAATCCTCTCA |
| | | ATCCCGAGCAAGCAGTTCTGAAGGCACAGGTGTGTGTGTAG |
| | | AGTGTGGATTTGTCTGTGTCTGTGTGCCTGTTGTGGGAGGGG |
| | | AAGCAGAAGGGGGCTGCTTCTTATCCTTGGAGACTTCTGTGG |
| | | AGAGGTGAAAAGGGAGGTGGGTGCGGGCGGCTGGAGAGAG |
| | | AGGAGACCTTGATTGTCCTGGGTCCTTAGAGATGCAGGGAA |
| | | GGAAAGTGTAAGGTGTGTGTGGGTGGGGTGAAGGTTTAGGG |
| | | GAGGAGAGCTGAGGGGTAAGGAAGGTTTGGGATAATGTGAG |
| | | GAGGCCGGTTCCAGACTGTCCCTGGCACACACCCTTCATGTA |
| | | ATCTCTGAAATAAAAGTGCGTGCTGTTTGTAAAAGCATTAGA |
| | | TTAAGTTCTAGGGGAATTGAGTAGACCTCTAAGGCACCTCTG |
| | | AAACTTCTTTAGGTATAAATTTCTTGCTAGTTTTTTGTTTTTT |
| | | AGTGTGTATATTTTTACATAGTAGAAATGACAGTGAAACTAA |
| | | CTTTTGAATTAAAGTTTTAACACAGTTACTGTATTATTATAAT |
| | | GCTAATAGTTTTCTAGTAGTTACATATTATTCTTTTATATATA |
| | | ATAGTTGTGACACAACTTAGGGATCACTTTCCCCTTTGTTGA |
| | | CCTTTATTATGACATTCACCAAAAGTTGAAAATGTATGTTTC |
| | | TGGTTAATTTTTAATTTATATTTTTTCATTTGAAAAAATGTGG |
| | | AGAGGTCTGTTTTTCATTCAAAAGAGTTCTTTTGAATTATTTT |
| | | GACCGATTTATTGGTCAATTTTAATAACTGCTCTAAGAATTC |
| | | CCTATTTTATTTGGTAGGTAATGGACAATGATCTACTGCCTA |
| | | ATATCTCTAGGGCTTAGTATTTTTCTCAGTGACTTTGTGGGTT |
| | | CTTTGTACTGTAAGATTATTAACACTTTATTGATATTTGATTC |
| | | AGCATTTGCTCCAGTTTGTGGTTTTTATGTTGATTTTGAAAAT |
| | | TCTTTTCCATGTTAAGAATTTGAACATTTTTATTTAATAAAAT |
| | | GTATTGCAAAAATTTTATTAATGATTTACAATCCATCTTAAA |
| | | TCTGCCATTTTGTGGTATTTTTGTCTCTAGGTTTCTCCTTACTT |
| | | CTAAAAACATTGTATTTATTGAGAGTATGCTAATGTCGGGGA |
| | | TTTTCCTGGGCATAAGCATCACAAGTAATGAGTTCCAGACCC |
| | | TGCCTTAATCCAAATGTGATTCTGGAAAGAAAAATCATTTTA |
| | | CAATTGGCCTGATAATAATTCTGCTTGTGTTGCACGGGGGAT |
| | | ACATTGAGCAGCTGAATGAAAATATAAGAACTTTCAAAACT |
| | | AAAATGACGCTCCTAAATCCTTCTGTCTGCTTTAGGACTCAT |
| | | GCTTTTCTAGGAACGTAAAAATTTGGAGAATCATTTCTGTCT |
| | | GTCCCACCTTCCCAGGGGCAGAACCATTTCTGTGGTGTTCTA |
| | | AGGTGTGAGTGCATGGCAGCAGTATTCCTAAAAATTCATACT |
| | | CTGTTTTCTCATGTACCCAACTCTGTCCCTTTGTCTATGTACA |
| | | TTGCTTTAAATCATATTTTTCTGTCACGGTGTACAAGGATGA |
| | | TAAATAGGTGCCAAGTGGAGCACCCAAGTGTGATGAGCCCC |
| | | CTTATGGTGGAATGGAGCGAGAAGCTTTCTGACCTCATAAAT |
| | | TGAAGGCTATCTTCACTCATTATGTATTTTACGTAAATTAATC |
| | | CTCATATAACCCCAAGAGGTAAATTAGTATAATTATCCTTCA |
| | | TTGGGGGTGACAAAGTTGAGACACAGAAGAATCAACTCTTC |
| | | CAGGATCAACCAGTAAAAGGCAGACCTTGGATTTGAACCAT |
| | | GCAACCTGGCTCAGATATCAGTTTTAATTACTACACTCTGTA |
| | | CTTTCAAAAATTTGTAAACACTTTGACAATGCATGCCAATTT |
| | | CAAGCTATGAAGAAGCAAACGCAATTTTTCACATCTCTCAAA |
| | | TCTAATGGGTCCTCACTATCAAGATTAAATTCCAGGCTGATG |
| | | ACACTGTAAGGCCACATGGCCAGCTGTGCTGGAGGCCTGTTC |
| | | AAGGTCAGAGCCTGGGTTTGCAGAGAAGCAGACAAACACCA |
| | | AAACCAGGAGAATTACTCTGTCTTCCTGACTCATTCCCTCTA |
| | | CCTTTTTTTCTACTAGTCCATCCTCAGGTGACTGTGTATCCTG |
| | | CAAAGACCCAGCCCCTGCAGCACCACAACCTCCTGGTCTGTT |
| | | CTGTGAGTGGTTTCTATCCAGGCAGCATTGAAGTCAGGTGGT |
| | | TCCGGAATGGCCAGGAAGAGAAGACTGGGGTGGTGTCCACA |
| | | GGCCTGATCCATAATGGAGACTGGACCTTCCAGACCCTGGTG |
| | | ATGCTAGAAACAGTTCCTCGGAGTGGAGAGGTTTACACTTGC |
| | | CAAGTGGAGCACCCAAGCGTAACAAGCCCTCTCACAGTGGA |
| | | ATGGAGTGAGCAGCTTTCTGATTTCATACATTTCTCACCCAC |
| | | CAAGAAGGGGACTGTGCTAATCCCTGAGTGTCAGGTTTCTCC |
| | | TCTCCCACGTCCTATTTTCATTTGTTCCATGTTCTCATCTCCA |
| | | TCAGCACAGGTCACTGGGGATAGCCCTGTAACAGTGTGTAG |
| | | AAACACCTGTACCCCCTGTGGAATCAGTCATGCTTTTTTTTTT |
| | | TTGAACCTCCCCATGATGTCACAGGTCAGGGTCACCCGCTCT |
| | | CCCCAAGCTCCAGGCCCTGCTTTTGGGTCTGAGACTGAGTTT |
| | | CTGGTGCTGCTGCTCTGAGTTACTTGTTTTGATCCGGGAAGA |
| | | GGGGAAGCATAGGGGCCTACCTGACATGAGGGGAGTCTAAT |
| | | CTCAGCTCTACCTTTTATTAGCTCTGTCACTCTAGACAAACTA |
| | | CTTAGCTTCATTGAGTCTCAGGCTTTCTGTTGATCAGATGTTG |
| | | AACGCTTGCCTTACATCAATGCTGTAATATTTGAATGAGTTT |
| | | GATGTCGGAACCTTGTAACTGTTCAGTGTGATTTGAAATCCT |
| | | TTTTTTCTCCCGAAATGGCTAGTTATTTTAGTTCTTGTGGGGC |
| | | AGCCTTCTTCCCCATTTTCAAAGCTCTGAATCTTAGAGTCTCA |
| | | ATTAAAGAGGTTCAATTTGGAATAAACGTCACTAAACCTGG |
| | | CTTCCTCTCTCAGGAGCACGGTCTGAATCTGCACAGAGCAAG |
| | | ATGCTGAGTGGAGTCGGGGGCTTTGTGCTGGGCCTGCTCTTC |
| | | CTTGGGGCCGGGCTGTTCATCTACTTCAGGAATCAGAAAGGT |
| | | AAGGAGTCTTTGGTAGCTGGCTCTCTCCGTAGGCTTTTCTGG |
| | | AGGAGGAACTATAGCTTTGCTGAGGTTAGTTCTCAGTATATG |
| | | AGTGGCCCTGAATAAAGCCTTTCTTTCCCCAAACGGCTCTAA |
| | | TGTCCTGCTAATCCAGAAATCATCAGTGCATGGTTACTATGT |
| | | GAAAGCATAATAGCTTGTGGCCTGCAGAGACAAGAGGAAGG |
| | | TTAACAAGTAGGGGTCCTTTGGTTTGAGATCTTGGAGCAAAT |
| | | TAAGGAAGAGCCACTAAAGTTAATGGAATTACACTGGATCC |
| | | TGTGACAGACACTTCATGCTTCATGGGTCACATGGTCTGTTT |
| | | CTGCTCCTCTCTGCCCTGGTTGGTGTGGGTTTTGGTGTTAGAG |
| | | AACTCTCCGGTGGGAGATCTGGGACTGGGATATTGTGTTGGA |
| | | GGACAGATTTGCTTCAATATCTTTTAAGTGTATATCTTTTCCT |
| | | CTTTTTCCCAGGACACTCTGGACTTCAGCCAACAGGTAATAC |
| | | CTTTTAATCCTCTTTTAGACACAGATTCAGTTTCTCTAGTGAG |
| | | AGGTGAAGCCAGCTGGACTTCTGGGTTGGGTGGGGACTTGG |
| | | AGAACTTTTCTGTCTTACAAGAGGTTTCTAAATGCACCAATG |
| | | AGTGCTCTGTAAAAACACACCAATGAGTGCTCTGTAAAAATT |
| | | GACACTCTGTGGCTTGCTAGATGTTTGTAAGATGGACCAATC |
| | | AGCACTCTGTAAAATGGACCAATCCACACTCTGTAAAATGG |
| | | ACCAATCAGCACTCTTTAAAATGGACCAATCAGCAGGATAC |
| | | GGGTGGGGACAAATAAGGGAATAAAAGCTGGTCACCCTAGC |
| | | CAGCACCGGCAACCTGCTTAGGTCCTTTTCTATGCTGTGGAA |
| | | GGTCTGTTCTTTCACTCTTCACAATAAATCTTGTGCTCACTCT |
| | | TTGGGTCCGTGCCACCTTTAAGAGCTATAACACTCACTGCAA |
| | | GGGTCTGTGGCTTCACTCTTGAAGTCAGCCAGACCGTGAACC |
| | | CACCGGAAGGAACAAACTCTGGACACACTAGAATGGTGGTA |
| | | GAGGTGATAAGGCATGAGACAGAAATAATAGGAAAGACTTT |
| | | GGATCCAAATTTCTGATCAGGCAATTTACACCAAAACTCCTC |
| | | CTCTCCACTTAGAAAAGGCCTGTGCTCTGCAGGACTATTGGC |
| | | TCTGGGAGACTCAGGAACTTGTTTTTCTTCTTCCTGCAGTGTT |
| | | CTCATCTGAGTCCTTCAAAGAGGGGGAAAGAAACGTTAGTA |
| | | GAGCCAGGTTGAAAACAACACTCTCCTCTGTCTTTTGCAGGA |
| | | TTCCTGAGCTGAAGTGCAGATGACAATTTAAGGAAGAATCTT |
| | | CTGCCCCAGCTTTGCAGGATGAAAAGCTTTCCCGCCTGGCTG |
| | | TTATTCTTCCACGAGAGAGGGCTTTCTCAGGACCTGGTTGCT |
| | | ACTGGTTCAGCAACTGCAGAAAATATCCTCCCTTGTGGCTTC |
| | | CTCAGCTCCTGCCCTTGGCCTGAAGTCCCAGCATTGATGGCA |
| | | GCGCCTCATCTTCAACTTTTGTGCTCCCCTTTGCCTAA |
| 31 | HLA- | TAAGTCTGAGTGTCATTTCTTCAATGGGACGGAGCGGGTGCG |
| | DRB3 | GTACCTGGACAGATACTTCCATAACCAGGAGGAGTTCCTGC |
| | | GCTTCGACAGCGACGTGGGGGAGTACCGGGCGGTGACGGAG |
| | | CTGGGGCGGCCTGTCGCCGAGTCCTGGAACAGCCAGAAGGA |
| | | CCTCCTGGAGCAGAAGCGGGGCCGGGTGGACAATTACTGCA |
| | | GACACAACTACGGGGTTGGTGAGAGCTTCACAGTGCAGCGG |
| | | CGAGGTGAGCATGTCGGGGGGCGGGGCCTGGGTCCCTGTGA |
| | | GCTGGAAATCTGAGAGAGAGTGTGTGTGTGTGTGTGTGTGTG |
| | | TGTGTGAGAGAGAGAGAGAGAGAGGAAGAGACAGAGAGAG |
| | | GGAGCGCGCCATCTGTGAGCATTTAGAATCCTCTCAATCCCG |
| | | AGCAAGCAGTTATGAAGGCACAGGTGTGTGTGTAGAGTGTG |
| | | GATTGGTCTGTGTCTGTGTGCCTGTTGTGGGAGGGGAAGCAG |
| | | GAGGGGGCTGCTTCTTATCCTTGGAGACTTCTGTGGAGAGGT |
| | | GACAAGGGAGGTGGGTGCGGGCGGCTGGAGAGAGAGGAGA |
| | | CCTTGATTGTCCTGGGTCCTTAGAGATGCAGGGAAGGAAAG |
| | | TGTAAGGTGTGTGTGGGTGGGGTGAAGGTTTAGGGGAGGAG |
| | | AGCTGAGGGGTAAGGAAGGTTTGGGATAATGTGAGGAGGCC |
| | | GGTTCCAGACTGTCCCTGGCACACACCCTTCATGTAATCTCT |
| | | GAAATAAAAGTGCGTGCTGTTTGTAAAAGCATTAGATTAAG |
| | | TTCTAGGGGAATTGAGTAGACCTCTAAGGCACCTCTGAAACT |
| | | TCTTTAGGTATAAATTTCTTGCTAGTTTTTTGTTTTTCTAGTGT |
| | | GTATATTTTTACATAGTAGAAATGACAGTGAAACTAACTTTT |
| | | GAATTAAAGTTTTAACACAGTTACTGTATTATTATAATGCTA |
| | | ATAGTTTTCTAGTAGTTACATATTATTCTTTTATATATAATAG |
| | | TTGTGACACAACTTAGGGATCACTTTCCCCTTTGTTGACCTTT |
| | | ATTATGACATTCACCAAAAGTTGAAAATGTATGTTTCTGGTT |
| | | AATTTTTAATTTATATTTTTTCATTTGAAAAAATGTGGAGAG |
| | | GTCTGTTTTTCATTCAAAAGAGTTCTTTTGAATTATTTTGACC |
| | | GATTTATTGGTCAATTTTAATAACTGCTCTAAGAATTCCCTAT |
| | | TTTATTTGGTAGGTAATGGACAATGATCTACTGCCTAATATC |
| | | TCTAGGGCTTAGTATTTTTCTCAGTGACTTTGTGGGTTCTTTG |
| | | TACTGTAAGATTATTAACACTTTATTGATATTTGATTCAGCAT |
| | | TTGCTCCAGTTTGTGGTTTGTATGTTGATTTTGAAAATTCTTT |
| | | TCCATGTTAAGAATTTGAACATTTTTATTTAATAAAATGTATT |
| | | GCAAAAATTTTATTAATGATTTACAATCCATCTTAAATCTGC |
| | | CATTTTGTGGTATTTTTGTCTCTAGGTTTCTCCTTACTTCTAA |
| | | AAACATTGTATTTATTGAGAGTATGCTAATGTCGGGGATTTT |
| | | CCTGGGCATAAGCACCACAAGTAATGAGTTCCAGACCCTGC |
| | | CTTAATCCAAATGTGATTCTGGAAAGAAAAATCATTTTACAA |
| | | TTGGCCTGATAATAATTCTGCTTGTGTTGCACGGGGGATACA |
| | | TTGAGCAGCTGAATGAAAATATAAGAACTTTCAAAACTAAA |
| | | ATGACGCTCCTAAATCCTTCTGTCTGCTTTAGGACTCATGCTT |
| | | TTCTAGGAACGTAAAAATTTGGAGAATCATTTCTGTCTGTCC |
| | | CACCTTCCCAGGGGCAGAACCATTTCTGTGGTGTTCTAAGGT |
| | | GTGAGTGCATGGCAGTAGTATTCCTAAAAATTCATACTCGGT |
| | | TTTCTCATGTACCCAACTCTGTCCCTTTGTCTATGTACATTGC |
| | | TTTAAATCATATTTTTCTGTCACGGTGTACAAGGATGATAAA |
| | | TAGGTGCCAAGTGGAGCACCCAAGTGTGATGAGCCCCCTTA |
| | | CGGTGGAATGGAGTGAGAAGCTTTCTGATCTCATAAATTGA |
| | | AGGCTATCTTCAGTCATTATGTATTTTACATAAATTAATCCTC |
| | | ATATAACCCCAAGAGGTAAATTAGTATAATTATCCTTCATTG |
| | | GGGGTGACAAAGTTGAGACACAGAAGAATCAACTCTTCCAG |
| | | GATCAACCAGTAAAAGGCAGACCTTGGATTTGAACCATGCA |
| | | ACCTGGCTCAGGTATCAGTTTTAATTACTACACTCTGTACTTT |
| | | CAAAAATTTGTAAACACTTTGACAATGCATGCCAATTTCAAG |
| | | TTATGAAGAAGCAAACGCAATTTTTCACATCTCTCAAATCTA |
| | | ATGGGTCCTCACTATCAAGATTAAATTCCAGGCTGATGACAC |
| | | TGTAAGGCCACATGGCCAGCTGTGCTGGAGGCCTGGTCAAG |
| | | GTCAGAGCCTGGGTTTGCAGAGAAGCAGACAAACAGCCAAA |
| | | CCAGCAGAATTACTCTGTCTTCCTGACTCATTCCCTCTACCTT |
| | | TTTTTCTCCTAGTCCATCCTCAGGTGACTGTGTATCCTGCAAA |
| | | GACCCAGCCCCTGCAGCACCACAACCTCCTGGTCTGCTCTGT |
| | | GAGTGGTTTCTATCCAGGCAGCATTGAAGTCAGGTGGTTCCG |
| | | GAACGGCCAGGAAGAGAAGGCTGGGGTGGTGTCCACGGGCC |
| | | TGATCCAGAATGGAGACTGGACCTTCCAGACCCTGGTGATG |
| | | CTAGAAACAGTTCCTCGGAGTGGAGAGGTTTACACTTGCCA |
| | | AGTGGAGCACCCAAGCGTAACGAGCGCTCTCACAGTGGAAT |
| | | GGAGTGAGCAGCTTTCTGATTTCATACATTTCTCACCCACCA |
| | | AGAAGGGGACTGTGCTAATCCCTGAGTGTCAGGTTTCTCCTC |
| | | TCCCACGTCCTATTTTCATTTGCTCCATGTTCTCATCTCCATC |
| | | AGCACAGGTCACTGGGGATAGCCCTGTAACAGTGTGTAGAA |
| | | ACACCTGTACTCCCTGTGGAATCAGTCATGCTTTTTTTTTTTT |
| | | TGAACCTCCCCATGATGTCACAGGTCAGGGTCACCCGATCTC |
| | | CCCAAGCTCCAGGCCCTGCTTCTGGGTCTGAGACTGAGTTTC |
| | | TGGTGCTGCTGCTCTGAGTTACTTGTTTTGATCTGGGAAGAG |
| | | GGGAAGCATAGGGGCCTACCTGACATGAGGGGAGTCCAATC |
| | | TCAGCTCTGCCTTTTATTAGCTCTGTCACTCTAGACAAACTAC |
| | | TTAGCTTCACTGAGTCTCAGGCTTTCTGTGGATCAGATGTTG |
| | | AACGCTTGCCTTACATCAAGGCTGTAATATTTGAATGAGTTT |
| | | GATGTCGGAACCTTGTAACTGTTCAGTGTGATTTGAAATCCT |
| | | TTTTTTCTCCCAAAATGGCTAGTTATTTTAGTTCTTGTGGGGC |
| | | AGCCTTCTTCCCCATTTTCAAAGCTCTGAATCTTAGAGTCTCA |
| | | ATTAAAGAGGTTCAATTTGGAATAAGCATCACTAAACCTGG |
| | | CTTCCTCTCTCAGGAGCACGGTCTGAATCTGCACAGAGCAAG |
| | | ATGCTGAGTGGAGTCGGGGGCTTTGTGCTGGGCCTGCTCTTC |
| | | CTTGGGGCCGGGCTGTTCATCTACTTCAGGAATCAGAAAGGT |
| | | GAGGAGCCTTTGGTAGCTGGGCCTTTCCATAGGCTTTTCTGC |
| | | AGGAGGAAATAGAGCTTTGCTGAGGTTAGTTCTCAGTATATG |
| | | AGTGGCTCTGAATAAAGCCTTTCTTTCCCCAAACGGCTCTAA |
| | | TGTCCTGCTAATCCAGAAATCATCAGTGCATGGTTACTATGT |
| | | GAAAGCATAATAGCTTGTGGCCTGCAGAGACAAGAGGAAGG |
| | | TTAACAAGTAGGGGTCCTTTGGTTTGAGATCTTGGAGCAAAT |
| | | TAAGGAAGAGACACTAAAGTTAATGGAATTACACTGGATCC |
| | | TGTGACAGACACTTCATGCTTCATGGGTCACATGGTCTGTTT |
| | | CTGCTCCTCTCTGCCCTGGTTGGTGTGGGTTTTGGTGTTAGAA |
| | | CTCTCCGGTGGGAGATCTGGGACTGTGATATTGTGTTGGAGG |
| | | ACAGATTTGCTTCAATATCTTTTAAGTGTATATCTTTTCCTCT |
| | | TTTTCCCAGGACACTCTGGACTTCAGCCAACAGGTAATACCT |
| | | TTTAATCCTCTTTTAGACACAGATTCAGTTTCTCCAGTGAGA |
| | | GGTGAAGCCAGCTGGACTTCTGGGTTGGGTGGGGACTTGGA |
| | | GAACTTTTCTGTCTTACAAGAGGTTTCTAAATGCACCAATGA |
| | | GTGCTCTGTAAAAACACACCAATGAGTGCTCTGTAAAAATTG |
| | | ACACTCTGTGGCTCGCTAGATGTTTGTAAGATGGACCAATCA |
| | | GCACTCTGTAAAATGGACCAATCCACACTCTGTAAAATGGA |
| | | CCAATCAGCACTCTTTAAAATGGACCAATCAGCAGGATACG |
| | | GGCGGGGACAAATAAGGGAATAAAAGCTGGTCACCCTAGCC |
| | | AGCACCTGCAACCTGCTTAGGTCCTTTTCTATGCTGTGGAAG |
| | | TTTTGTTCTTTTGATCTTCACAATAAATCTTGTGCTCACTCTT |
| | | TGGGTCCGTGCCACCTTTAAGAGCTATAACACTCACTGCAAG |
| | | GGTCTGTGGCTTCACTCTTGAAGTCAGCCAGACCCTGAACCT |
| | | ACCGGAAGGAACAAACTCAGGACACACTAGAATGATGGTAG |
| | | AGGTGATAAGGCATGAGACAGAAATAATAGGAAAGACTTTG |
| | | GATCCAAATTTCTGATCAGGCAATTTACACCAAAACTCCTCC |
| | | TCTCCACTTAGAAAAGGCCTGTGCTCTGCAGGACTATTGGCT |
| | | CTGGGAGACTCAGGAACTTGTTTTTCTTCTTCCTGCAGTGTTC |
| | | TCATCTGAGTCCTTCAAAGAGGGGGAAAGAAACGTTAGTAG |
| | | AGCCAGGTTGAAAACAACACTCTCCTCTGTCTTTTGCAGGAT |
| | | TCCTGAGCTGAAGTGCAGATGACAATTTA |
| 32 | HLA- | GTTCGTTGCAACAAATTGATGAGCAATGCTTTTTTATAATGC |
| | DRB4 | CAACTTTGTACAAAAAAGTTGGCATGGTGTGTCTGAAGCTCC |
| | | CTGGAGGCTCCTGTATGGCAGCGCTGACAGTGACATTGACG |
| | | GTGCTGAGCTCCCCACTGGCTTTGGCTGGGGACACCCAACCA |
| | | CGTTTCTTGGAGCAGGCTAAGTGTGAGTGTCATTTCCTCAAT |
| | | GGGACGGAGCGAGTGTGGAACCTGATCAGATACATCTATAA |
| | | CCAAGAGGAGTACGCGCGCTACAACAGTGACCTGGGGGAGT |
| | | ACCAGGCGGTGACGGAGCTGGGGCGGCCTGACGCTGAGTAC |
| | | TGGAACAGCCAGAAGGACCTCCTGGAGCGGAGGCGGGCCGA |
| | | GGTGGACACCTACTGCAGATACAACTACGGGGTTGTGGAGA |
| | | GCTTCACAGTGCAGCGGCGAGTCCAACCTAAGGTGACTGTG |
| | | TATCCTTCAAAGACCCAGCCCCTGCAGCACCACAACCTCCTG |
| | | GTCTGCTCTGTGAATGGTTTCTATCCAGGCAGCATTGAAGTC |
| | | AGGTGGTTCCGGAACGGCCAGGAAGAGAAGGCTGGGGTGGT |
| | | GTCCACAGGCCTGATCCAGAATGGAGACTGGACCTTCCAGA |
| | | CCCTGGTGATGCTGGAAACAGTTCCTCGGAGTGGAGAGGTTT |
| | | ACACCTGCCAAGTGGAGCATCCAAGCATGATGAGCCCTCTC |
| | | ACGGTGCAATGGAGTGCACGGTCTGAATCTGCACAGAGCAA |
| | | GATGCTGAGTGGAGTCGGGGGCTTTGTGCTGGGCCTGCTCTT |
| | | CCTTGGGACAGGGCTGTTCATCTACTTCAGGAATCAGAAAG |
| | | GACACTCTGGACTTCAGCCAACAGGACTCTTGAGCTGCCCAA |
| | | CTTTCTTGTACAAAGTTGGCATTATAAGAAAGCATTGCTTAT |
| | | CAATTTGTTGCAACGAAC |
| 33 | HLA- | TAAGTGTGAGTGTCATTTCCTCAATGGGACGGAGCGAGTGTG |
| | DRB4 | GAACCTGATCAGATACATCTATAACCAAGAGGAGTACGCGC |
| | | GCTACAACAGTGACCTGGGGGAGTACCAGGCGGTGACGGAG |
| | | CTGGGGCGGCCTGACGCTGAGTACTGGAACAGCCAGAAGGA |
| | | CCTCCTGGAGCGGAGGCGGGCCGAGGTGGACACCTACTGCA |
| | | GATACAACTACGGGGTTGTGGAGAGCTTCACAGTGCAGCGG |
| | | CGAGGTGAGCATGGTGGAGGGCGGGGCCTGGGTCCCTGTGA |
| | | GCGGGGAATCTGTGTATGTGTGTGTGTGTGTGTGTGTGTGAG |
| | | AGAGAGAGAGAGAGAGAGAGACAGAGAAAGAGAGAGAGA |
| | | GCGTGCGCCATCTGTGAGCATTTAGAATCCTCTCTATCCTTTC |
| | | TTATGGCTGCGCAGTATTCCCTGGTGTATAGGTGCCACATTG |
| | | TCTTAATCCAGTCTATCATTGATGGACATTAGGGTTAATTCC |
| | | AAGTCTTTGCTATTGTGAATAGTGTCAGTTCATGTCCTTTGTA |
| | | GGGACATGGATGCAGCTGGAAACCATCATTCTCAGCAAACT |
| | | ATCACAAGGACAGAAAACCAAACACCACTTGTTCTCACTCA |
| | | TAGGTGGGAATTGAACAATGAGAACACATGGACACAGGAAG |
| | | GGGAACATCACACACAGGGGCCTGTCGTGGGGTGGGGGGAG |
| | | CAGGGAGTGATAGCATTAGGAGATATACCTAATGTAAATGA |
| | | CTAGTTAATGGGTGCAGCATACCAACATGACACATGTGTAC |
| | | ATATGTAACAAACCTGCACGTTGTGCACATGTATCCTAGAAC |
| | | TTAAAGTATAATAATAATAATTATATATATATATATATATTA |
| | | GAAAAACAAACAAAAAGAATCTTCTGTATCCTGAGCAAGGA |
| | | GTTCTGAAGGCACAGGTGTGTGTAGAGTGTGGATTTGTCTGT |
| | | GTGGCTGTTGTGGGAGGGGAAACAGGAGGGGGCTGCTTCTT |
| | | ATTCTTGGAGGCCTCTGTGCAGAGGTGACATGGGAGGTGGG |
| | | TGTAGGGGGCTGGAGAGAGAGGAGACCTTGATTGTCCTGGG |
| | | TCCTTAGAGATGCAGGGAAGGGAAGAATAAGGTGTGTGTGG |
| | | TTGGGGTGAAGGTTTAGGGGAGGAGAGCTGAGGAGTGTGGA |
| | | AGGTTTGGGATAATGTGAGGAGGCCAGTTCCAGACTGTCCCT |
| | | GGCACACACCCTTCATGTAATCTCTGAAATAAAAGTGTGTGC |
| | | TGTTTGTTTGTAAAAGCATTAGATTAATTTCTAGGGGAATTG |
| | | AGGAGACCTCTGAGGCACCTCTGAAGCTTCTTTAGGTCTAAA |
| | | TTTCTTGCTAGTTTTTGTTTTTTTAGGGTGTATATTTTTACATA |
| | | GCAGAAGTGACTGTGAAACTAACTTTTTGAATTAAAGTTTTA |
| | | ACACAGTTACTATTTTATTATAATGCTAATAGTTTTCTAGTAG |
| | | TTACATCTTATTCTTTTGTATATAATAGTTGTGACACAACTTA |
| | | CCTCACTTTCCCCTTTTTGTTGACCTTTATTATGACTTTCACC |
| | | AAAAGTTGAAAATGTATGTTTCTGGTTAATTTTTACTTTATAT |
| | | TTTTCATTTGTAATTCTTTTGAATTATTTTGACCTATTTATTGG |
| | | CCAATTATAATTACTGCTGTAAGAATTCCCTATTTCATTTGGT |
| | | AGGGAATGTACAATGATCTACTGTCTAATATCTTAAGGGCTT |
| | | AGTATTTTTCTCAGTGACTTTGTGGGTTCTTTGTACTGTAAGA |
| | | TTATTAACACTTTATTGATATTTGATTCAGCATTTTCTCCAGT |
| | | TTGTGGTTTGTATGTGGATTTTGAAAAGTATTTTCCATGTTAA |
| | | GAATTTGAACATTTTTATTTAATAAAATATATTGCGAAATTT |
| | | GTATTAATGATTTACAATCATCTTAAATCCACCATTTTGTGGT |
| | | ATTATTGTCTCCAGGTTTCTCCTTACTTCTAAAAAAAAACTGT |
| | | ATTTATTGAGAGTATGCTAGTGTCAGGGATTTTCCTGGGCAT |
| | | AAGCACCCCAAGTGACGAGTCCCAGACACTGCCTTAATCCA |
| | | AATGTCATTCTGGAAAGAAAAATCATTTTACAATGATAGGCC |
| | | TAATAATAATTAAGCTTGTGTTGCACAGGAGATACATTGATC |
| | | AGCTAAATGTAAACGTAAGAACTTTCAAAACTAAAATGACA |
| | | TTCCTTAATCATTCTCTCTGCGTTAGGACTCATGCTTTTCTAG |
| | | GAACGTAAAAATTTGAGAATCATTTCTGTCTGTCCCACCTTC |
| | | CCAGGGGCAGAACCATTTCTGTGGTGTTCTAAGGTGTGAGTG |
| | | CATGGTGGTAGTATTCCCAAAAATTCATACTCGGTTTCCTCA |
| | | TGTACCCAACTCTGTCCCTTTATCTACCCACATTGCTTTAAGT |
| | | CATATTTTTCTGTCACGGTGTACAAGGATGATAAATAGGTGC |
| | | CAAGTGGAGCACCCAAGTGTGATGAGCCCCCTCACAGTGGA |
| | | ATGGAGTGACAAGCTTTCTAACCTCATAAATTGAAGGCTATC |
| | | TTCAGTCATTGTTTTATATATTTTACGTGCATTAATCCTCATA |
| | | TAACCCCAAGAGGTAAATTAGTATAATTATCCTTCATTGTAG |
| | | GTGACAAAGTTGAGACACAGAAGAATCAAAAAACTCTTCCA |
| | | GCATCAACCAGTAAAAGGCAGACCTTGGATTTCAACCAGGC |
| | | AACATGGCTCAGATATCAGTTTTAATTACTACACTCTACTGT |
| | | CAAAGATTTGTAAACACTTTGACAATGCATGCCAATTTCAAG |
| | | CTATGAAGAAACAAACACAATTTTTCACAACATCTCTCAAAT |
| | | CTAATGGGTCCTCACTATCAAGATTAAATTCCAGGCTGATGA |
| | | CACTGTAAGGCCACATGGCCAGCTGTGCTGGAGGCCTGGTC |
| | | AAGGTCAGAGCCTGGGTTTGCAGAGAAGCAGACAAACAGCC |
| | | AAACAAGGAGACTTACTCTGTCTTCATGACTCATTCCCACTA |
| | | CCTTTTTTTTCTCCTAGTCCAACCTAAGGTGACTGTGTATCCT |
| | | TCAAAGACCCAGCCCCTGCAGCACCACAACCTCCTGGTCTGC |
| | | TCTGTGAATGGTTTCTATCCAGGCAGCATTGAAGTCAGGTGG |
| | | TTCCGGAACGGCCAGGAAGAGAAGGCTGGGGTGGTGTCCAC |
| | | AGGCCTGATCCAGAATGGAGACTGGACCTTCCAGACCCTGG |
| | | TGATGCTGGAAACAGTTCCTCGGAGTGGAGAGGTTTACACCT |
| | | GCCAAGTGGAGCATCCAAGCATGATGAGCCCTCTCACAGTG |
| | | CAATGGAGTTAGCAGCTTTCTGACTTCCTAAATTTCTCACCC |
| | | ACCAAGAAGGGGACTGTGCTAATCCCTGAGTGTCAGGTTTCT |
| | | CCTCTCCCACATCCTATTTTCATTTGCTCCATGTTCTCATCTC |
| | | CATCAGCACAGGTCACTGGGCTACTGGGGGTAGCCCTGTAG |
| | | GTGTTTCTAGAAACACCTGTACCCCCTGGGGAATCAGTCACG |
| | | CCTGCCAGGCGGGAGAGGCTGTCCCTCTTTTGAACCTCCCCA |
| | | TGATGTCCCAGTTCAGGGTCACCCACTCTCCCCAGGCTCCAG |
| | | GCCCTGCCTCTGGGTCTGAGACTGCGTTTCTGGTGCTGTTGA |
| | | TCTGAGTTGTTTGTTGTGAGAAGAGGAGAACTCTAGGGGCCT |
| | | TCCTGACATGAGGGGAGTCCAATCTCAACTCTGCCTTTTATT |
| | | AGCTCTGTCACTCTAGACAAACTACTTAACCTCACTGAGACT |
| | | CAGGCTTTCTGTTGATCAGATTTTGAAGTTGCGCCTTACATC |
| | | AAGGCTGTAATATTTGAAATGAGTTTGATGCCTGGACCTTGT |
| | | AACTGTTACAGTGTGATTTGAAAACCGTTTTTTCCCCCAGAA |
| | | ATGGCTAGTTATTTTAGTTCTTACAGGGCAGCCTTCTTTCCCA |
| | | TTTTGAAAGCTCTGAATCTTAGGGTCTCAATTACAGAGGTTC |
| | | AATTTGGAATAAACACTAAACCTGGCTTCCTCTCTCAGGTGC |
| | | ACGGTCTGAATCTGCACAGAGCAAGATGCTGAGTGGAGTCG |
| | | GGGGCTTTGTGCTGGGCCTGCTCTTCCTTGGGACAGGGCTGT |
| | | TCATCTACTTCAGGAATCAGAAAGGTGAGGAGCTTTTGGGA |
| | | GCTGAATCTCCATAGGCTTTTCTGGAGGAGGAACTATGACTT |
| | | TGCTAGGGTTAGTTCTCAGTATATCAGTGGCCCTGGATAAAG |
| | | CCTTTCTTTCCCCAAATGACCTCCAATGTCCTGATAATCCAG |
| | | AAATCATCAGTGCATGGTTACTATGTCAAAGCATAATAGCTT |
| | | GTGGCCTGCAGAGATAAGAGAAAGGTTAACAAGTAGGGATT |
| | | CTTTGGTTGGAGATCCTGGAGCAAATTAAGGAAGAGCCACT |
| | | AAGGCTAATGCAATTACGCTGGATCCTGTGACAGACACCTC |
| | | ATGCTTCATGGGTCACATGGTCTGTTTCTGTTCCTCTCTGCCC |
| | | TGGTTGGTGTGGGTTGTGGTGTTAGAGAAATCTCAGGTGGGA |
| | | GATCTGGGGCTGGGACATTGTGTTAGAAGACAGATTTGCTTC |
| | | CATATCTTTTAAGTGTATATCTTTTCCTCTTTTTCCCAGGACA |
| | | CTCTGGACTTCAGCCAACAGGTAATACCTTTTAATCCTCTTTC |
| | | AGAAAGAGATTCAGTTTCCCTAGAATGATGGCAGAGGTGAT |
| | | AAGGCATGAGACAGAAATAATAGGAAAGACTTTGGATCCAG |
| | | ATTTCTGATCAGGCAATTTACCCCAGAACTCCTCCTCTCCAC |
| | | TTAGAAAAGGCCTGTGCTTTGCAGGATCATTGGCTCAGGGA |
| | | GACTTAGGAACTTGTTTTTCTTCTCCCTGCAGTGCTCTCATCT |
| | | GAGTCGTTGAAAGCGGGGAAAAGAAGTTTTAGTAAAGCCAG |
| | | GTCTGAAAACAATTTCCTCTGTCTCTGCAGGACTCTTGAGCT |
| | | GAAGTGCAGATGACCACATTCAAGGAAGAACCTTCTGCCCC |
| | | AGCTTTGCAAGATGAAAAGCTTTCCCACTTGGCTCTTATTCT |
| | | TCCACAAGAGCTTTGTCAGGACCAGGTTGTTACTGGTTCAGC |
| | | AACTCTGCAGAAAATGTCCTCCCTTGTGGCTTCCTTAGCTCC |
| | | TGTTCTTGGCCTGAAGCCTCACAGCTTTGATGGCAGTGCCTC |
| | | ATCTTCAACTTTTGTGCTTCCCTTTACCTAA |
| 34 | HLA- | CACGTTTCTTGCAGCAGGATAAGTATGAGCGTCATTTCTTCA |
| | DRB5 | ACGGGACGGAGCGGGTGCGGTTCCTGCACAGAGACATCTAT |
| | | AACCAAGAGGAGGACTTGCGCTTCGACAGCGACGTGGGGGA |
| | | GTACCGGGCGGTGACGGAGCTGGGGCGGCCTGACGCTGAGT |
| | | ACTGGAACAGCCAGAAGGACTTCCTGGAAGACAGGCGCGCC |
| | | GCGGTGGACACCTACTGCAGACACAACTACGGGGTTGGTGA |
| | | GAGCTTCACAGTGCAGCGGCGAGGTGAGCATGGTGGGGGGC |
| | | GGGGCCTGGGTCCTTGTGAGCTGGGAATCTGAGTGTGTGTGT |
| | | GTGTGTGTGTGTGTGTGTGTGTGTGTGAGAGAGAGAGAGAG |
| | | AGGAAGAGAGAGACAGAGAGGGAGCGCGCCATCTGTGAGC |
| | | ATTTAGAATCCTCTCAATCTTGAGCAAGGACTTCTGAGGACA |
| | | CAGGTGTGTGTGTAGAGTGTGGATTTGTCTTTGTGGCCTTTG |
| | | TGGGAGGGGAAGCAGGAGGGGGCTTCTTCTTATCCTTGGAG |
| | | GCCTCTGTGGGGAGGTGACATGGGAGGTGGGTGCAGGGGGC |
| | | TGGAGAGAGAGGAGACCTTGGTTGTCTCGTGTCCTTAGAGAT |
| | | GCAGGGAAGGAAAGTGTAAGGTGTGTGTGGTTGGGGTGAAG |
| | | GTTTAGGGGAGGAGAGCTGAGGGGTGTGGAAGATTTGTGAT |
| | | AATATGAGGAGGCCAGTTCCAGACTGTCCCTGGCACCCACC |
| | | CTTCACGCAGTCTCTGAAATAAAAGTGTGTGGTGTTTGTTTG |
| | | CAAAAGCATTAGATTAATTTCTAGGGGAATTGAGGAGACCT |
| | | CTGAGGCACTTCTGAAGCTTCTTTAGGTCTAAATTTCTTGCTA |
| | | TTTTTTCTGTTTGTTTTCTTAGTGTGTATATTTTTACATAGTTG |
| | | AAATGACTGTGAAACTAACTTTTTGATTAAAATTTTAATACA |
| | | CTGTTACTATTTTATTATAATGCTAATAGTTTTCTACTACTTA |
| | | CCTATTATTCTTTTATATATAATAGTTGTGACACAACTTATCT |
| | | CACTTTCCCCTTTGTTGACCTTTATTATGACATTCACCAAAAG |
| | | TTGAAAATATATGTTTCTGGTTATTATTTAATTTATTTTTTTA |
| | | TTTGGAATTCTTTTGAATTATCTTGACCTATTTATTGCCCAAT |
| | | TATAATTACTGCTCTAAGAATTCCCTATTGTATTTGGTAGGT |
| | | AATGGACAATGATCTATTTTCTGTTATCTCCAGGGCTTAGTA |
| | | TTTTTCTCAGTGATTTTGTGGGTTCTTTGTACTGTAAGATTAT |
| | | TAACACTTTATTGATATTTGATTCCACATTTTCTCCAGTTTTT |
| | | GATTTGTATGTTGATTTTGAAAATTCTATGTTAAGAATTTGA |
| | | ACATTTCTGTTTAATAAAATATATTCCAAAACTTTTATTAATG |
| | | GTTTACAAACCATCTTAAATCTATCATTTTGTGGTATTTTTGT |
| | | CTCCAGGTTTCTCCTTCCTTCTTAAAAAAAATGTATTTATTGA |
| | | GAGTCTGCTAGTGTTAGGGATTTTCCTAGGCATAAGCACCCC |
| | | AAGTAATGAGTCCCAGACCCTGCCTGCCTTGATCCAAATGTC |
| | | ATTCTGGAAAGAAAAATTATTTTACAATGATAGTATAATAAT |
| | | AGTTATGCTTGTTTGCATGGGAGATGCATTGATCAGCTAAAT |
| | | GTAAATGTAAGAATTTTCAAAACTAAAATAACTTTCCTTAAT |
| | | CCTTCTCTCTGCTTTATGACTCATGCTTTGCTGGGAACTTAAA |
| | | GATTGGAGAACCATTTCTGTCTGTCCTACCTTCCCAGGGGCA |
| | | CAACCATTTCTGTGTTGTTCTAAGGTGTGAGTGCATGGCAGT |
| | | AGTATTCCTAAACATTCATACTCAGTCTCCTTTTGTACCCTAC |
| | | TCGGTCCCTTTATCTATCCACATTACTTTAAATCATATTTTTC |
| | | TCTCAAGCTGTACAAGGATGATAAATAGGTGGCAAGTGGAA |
| | | CACCCAAGTGTGATGAGCCCTCTCACAGTGGAATGGAGTGA |
| | | GAAGCTTTATGACCTCATAAATTAAAGGCTATCTTCAGTCAT |
| | | TGTTTTATATATTTTATGTGCATTAATCCTCATATAACCCCAA |
| | | GAGGTAAATTACTATAATTATCCTCTATTATGGGTGAGAAAG |
| | | TTGAGACACAGAAGAATCGAAAAACTCTTCCAGCATCAACC |
| | | AGTAAAAGGCAGACCTTGGATTTGAGCCAGGCAACCTGGCT |
| | | CAGGTATCAGTTTTAATTACTACACTCTGTACTTTCAAAGAC |
| | | TTGTAAACACTTTGACAATGCATCCCAATTTCAAGTGATGAA |
| | | GAAACAAACACAATTTTTCACATCTCTCAAATCTGATGAGCC |
| | | CCCACTATAAAGACTAAATTCCAGGCTGATGACACTGTGAG |
| | | GCCTCATGGCCAGCTGTGCTGGAGGCCTGGTCAAGGCCAGA |
| | | GCCTGGGTTTACAGAGAAGCAGACAAACAGCCAAAAAGGG |
| | | AGATTCACTCTGTCTTCCTGAGTCATTCCCTCTACATTTCCTT |
| | | TCTCCTAGTTGAGCCTAAGGTGACTGTGTATCCTGCAAGGAC |
| | | CCAGACCCTGCAGCACCACAACCTCCTGGTCTGCTCTGTGAA |
| | | TGGTTTCTATCCAGGCAGCATTGAAGTCAGGTGGTTCCGGAA |
| | | CAGCCAGGAAGAGAAGGCTGGGGTGGTGTCCACAGGCCTGA |
| | | TTCAGAATGGAGACTGGACCTTCCAGACCCTGGTGATGCTGG |
| | | AAACAGTTCCTCGAAGTGGAGAGGTTTACACCTGCCAAGTG |
| | | GAGCACCCAAGCGTGACGAGCCCTCTCACAGTGGAATGGAG |
| | | TGAGCAGCTTTCTGACTTCATAAATTTCTCAACCACCAAGAA |
| | | GCAAACTTTACTAATCCCTGAGTGTCAGGTTTCTCATCTCCC |
| | | ACATCCTATTTTCATTTGCTCCATGTTCTCATCTCCATTAGCA |
| | | CAGGTCACTGGGGGTAGCCCTGTATAGTTTCTAGAAACACCT |
| | | GTATCCTCTGGGGAAGCAGTCATTCCTGGCAGGAAGGAGAG |
| | | GCTGTCCCTGTTTTGAACCTCCCCATGATGTCACAGGTCAGG |
| | | GTCACCCCCTCTCCCAGGGCTCCAGACCCTGCCTCTGGGTCT |
| | | GAGACTGTGTTTCTGGTGCTGTTGATCTGAGTTATTTGTTGTG |
| | | ATCTGGGAAGAGGAGAAGTGTAGGGGCCTTCCTGACATGAG |
| | | GGGAGTCCAATATCAGCTCTGCCTTTTATTAGCTCTGTCACT |
| | | CTAGACAAACTATTTAACCTCATTGAGTCTCAGGCTTTCTGT |
| | | TTATCAGATGTTGAAGCCGTGCCTTACATCAGGGCTGTAATA |
| | | TTAGAATGAATTTGATCCCTGAAACTTGTAACTGTTCAGTGT |
| | | GATTTGAAAACCTTTTTTTTCTCCAGAAATGGCTAGTTATTTT |
| | | AGTTCTTACAGAGCAGCCTTCTTCCCCATTTTCAAAGCTATG |
| | | AATATTGCAGGGTCTCAATTAAAAAGGTTCAATTTGGGATAA |
| | | AAATCACTAAACCTGGTTTCCTCTCCCAGGAGCACAGTCTGA |
| | | ATCTGCACAGAGCAAGATGCTGAGTGGAGTCGGGGGCTTTG |
| | | TGCTGGGCCTGCTCTTCCTTGGGGCCGGGCTATTCATCTACTT |
| | | CAAGAATCAGAAAGGTGAGGAGCCTTTGGTAGCGGCTCTCT |
| | | CCATAGACTTTTCCAGAGGAGGAAATAGGGCTTTGCTGAGG |
| | | TTAGTTCTCAGTATATGAGTGGCCCTGGATAAAGCCTTTCTT |
| | | TCCACAAATGACCTCCAATGCCCTGCTAATCCAGAAATCATT |
| | | AATGCATGGTTACTATGTCAAAAGCATAATAGCTTGTGGCCT |
| | | GCAGAGATAAGAGAAAGGTTAACTAGTTAAGGGTCCTTTGG |
| | | TTTGAAATCCTGGAGCAAATTAAGGAAGAGCCACTAAGGCT |
| | | AATGCAATTACACTGGATCCTGTGACAGACATGTCATGCTTC |
| | | ATGGGTCACATGGTCTGTTTCTGCTCCTCTCTGCCCTGGTTGG |
| | | TGTGGGTTGTGGTGTTAGAGAAATCTCAGGTGGGAGATCTG |
| | | GGACTAGGACATTGTGTTGGGAGAATAGATTTGCTTCCATAC |
| | | CTTTTAAGTGTATATCTTTTCCTCTTTTTCCCAGGGCACTCTG |
| | | GACTTCACCCAACAGGTAATACCTTTTAATCCTCTTTTAGAA |
| | | ACAGATTCAGTTTTCCTAGAATGATGGCAGAGGTGATAAGG |
| | | CATGAGACAGAAATAGCAGGAAAGACTTTGGATCCAAATTC |
| | | CTGATCAGGCAATTTATACCAAAACTCCTCCTCTCCACTTAG |
| | | GCCTGTGCTCTGCAGGAGTATTGGTTCAGGGAGACTTAGGA |
| | | ACTTGTTTTTCTTCTTCCTGCAGTGCTCTCATCTGAGTCCTTG |
| | | AAAGAGAGGAAAAGAAGCTGTTAGTGGAACCAGGTCTGAA |
| | | AACAACACTTTCCTCTCTCTCTGCAG |
| 35 | HLA- | TAAGTATGAGTGTCATTTCTTCAACGGGACGGAGCGGGTGC |
| | DRB5 | GGTTCCTGCACAGAGGCATCTATAACCAAGAGGAGAACGTG |
| | | CGCTTCGACAGCGACGTGGGGGAGTACCGGGCGGTGACGGA |
| | | GCTGGGGCGGCCTGACGCTGAGTACTGGAACAGCCAGAAGG |
| | | ACATCCTGGAGCAGGCGCGGGCCGCGGTGGACACCTACTGC |
| | | AGACACAACTACGGGGCTGTGGAGAGCTTCACAGTGCAGCG |
| | | GCGAGGTGAGCATGGTGGGGGGCGGGGCCTGGGTCCTTGTG |
| | | AGCTGGGAATCTGAGTGTGTGTGTGTGTGTGTGTGTGTGTGT |
| | | GTGAGAGAGAGAGAGAGAGAGAGAGGAAGAGAGAGACAG |
| | | AGAGGGAGCGCGCCATCTGTGAGCATTTAGAATCCTCTCAAT |
| | | CTTGAGCAAGGACTTCTGAGGACACAGGTGTGTGTGTAGAG |
| | | TGTGGATTTGTCTTTGTGGCCTTTGTGGGAGGGGAAGCAGGA |
| | | GGGGGCTTCTTCTTATCCTTGGAGGCCTCTGTGGGGAGGTGA |
| | | CATGGGAGGTGGGTGCAGGGGGCTGGAGAGAGAGGAGACC |
| | | TTGGTTGTCTCGTGTCCTTAGAGATGCAGGGAAGGAAAGTGT |
| | | AAGGTGTGTGTGGTTGGGGTGAAGGTTTAGGGGAGGAGAGC |
| | | TGAGGGGTGTGGAAGATTTGTGATAATATGAGGAGGCCAGT |
| | | TCCAGACTGTCCCTGGCACCCACCCTTCACGCAGTCTCTGAA |
| | | ATAAAAGGGTGTGGTGTTTGTTTGCAAAAGCATTAGATTAAT |
| | | TTCTAGGGGAATTGAAGAGACCTCTGAGGCACCTCTGAAGC |
| | | TTCTTTAGGTCTAAATTTCTTGCTATTTTTTCTGTTTGTTTTCT |
| | | TAGTGTGTATATTTTTACATAGTTGAAATGACTGTGAAACTA |
| | | ACTTTTTGATTAAAATTTTAATACACTGTTACTATTTTATTAT |
| | | AATGCTAATAGTTTTCTACTACTTACCTATTATTCTTTTATAT |
| | | ATAATAGTTGTGACACAACTTATCTCACTTTCCCCTTTGTTGA |
| | | CCTTTATTATGACATTCACCAAAAGTTGAAAATATATGTTTC |
| | | TGGTTAATATTTAATTTATTTTTTTATTTGGAATTCTTTTGAA |
| | | TTATCTTGACCTATTTATTGCCCAATTATAATTACTGCTCTAA |
| | | GAATTCCCTATTGTATTTGGTAGGTAATGGACAATGATCTAT |
| | | TTTCTGTTATCTCCAGGGCTTAGTATTTTTCTCAGTGATTTTG |
| | | TGGGTTCTTTGTACTGTAAGATTATTAACACTTTATTGATATT |
| | | TGATTCCACATTTTCTCCAGTTTTTGATTTGTATGTTGATTTT |
| | | GAAAATTCTATGTTAAGAATTTGAACATTTCTGTTTAATAAA |
| | | ATATATTCCAAAACTTTTATTAATGGTTTACAAACCATCTTA |
| | | AATCTATCATTTTGTGGTATTTTTGTCTCCAGGTTTCTCCTTC |
| | | CTTCTTAAAAAAAATGTATTTATTGAGAGTCTGCTAGTGTTA |
| | | GGGATTTTCCTAGGCATAAGCACCCCAAGTAATGAGTCCCA |
| | | GACCCTGCCTGCCTTGATCCAAATGTCATTCTGGAAAGAAAA |
| | | ATTATTTTACAATGATAGTATAATAATAGTTATGCTTGTTTGC |
| | | ATGGGAGATGCATTGATCAGCTAAATGTAAATGTAAGAATT |
| | | TTCAAAACTAAAATAACTTTCCTTAATCCTTCTCTCTGCTTTA |
| | | TGACTCATGCTTTCCTGGGAACTTAAAGATTGGAGAACCATT |
| | | TCTGTCTGTCCTACCTTCCCAGGGGCACAACCATTTCTGTGG |
| | | TGTTCTAAGGTGTGAGTGCATGGCAGTAGTATTCCTAAACAT |
| | | TCATACTCAGTCTCCTTTTGTACCCTACTCGGTCCCTTTATCT |
| | | ATCCACATTACTTTAAATCATATTTTTCTCTCAAGCTGTACAA |
| | | GGATGATAAATAGGTGGCAAGTGGAACACCCAAGTGTGATG |
| | | AGCCCTCTCACAGTGGAATGGAGTGAGAAGCTTTATGACCTC |
| | | ATAAATTAAAGGCTATCTTCAGTCATTGTTTTATATATTTTAC |
| | | GTGCATTAATCCTCATATAACCCCAAGAGGTAAATTACTATA |
| | | ATTATCCTCTATTATGGGTGAGAAAGTTGAGACACAGAAGA |
| | | ATTGAAAAACTCTTCCAGCATCAACCAGTAAAAGGCAGACC |
| | | TTGGATTTGAGCCAGGCAACCTGGCTCAGGTATCAGTTTTAA |
| | | TTACTACACTCTGTACTTTCAAAGACTTGTAAACACTTTGAC |
| | | AATGCATCCCAATTTCAAGTGATGAAGAAACAAACACAATT |
| | | TTTCACATCTCTCAAATCTGATGAGCCCCCACTATAAAGACT |
| | | AAATTCCAGGCTGATGACACTGTGAGGCCTCATGGCCAGCT |
| | | GTGCTGGAGGCCTGGTCAAGGCCAGAGCCTGGGTTTACAGA |
| | | GAAGCAGACAAACAGCCAAAAAGGGAGATTCACTCTGTCTT |
| | | CCTGAGTCATTCCCTCTACATTTCCTTTCTCCTAGTTGAGCCT |
| | | AAGGTGACTGTGTATCCTGCAAGGACCCAGACCCTGCAGCA |
| | | CCACAACCTCCTGGTCTGCTCTGTGAATGGTTTCTATCCAGG |
| | | CAGCATTGAAGTCAGGTGGTTCCGGAACGGCCAGGAAGAGA |
| | | AGGCTGGGGTGGTGTCCACAGGCCTGATTCAGAATGGAGAC |
| | | TGGACCTTCCAGATTCTGGTGATGCTGGAAACAGTTCCTCGG |
| | | AGTGGAGAGGTTTACACCTGCCAAGTGGAGCACCCAAGCGT |
| | | GACGAGCCCTCTCACAGTGGAATGGAGTGAGCAGCTTTCTG |
| | | ACTTCATAAATTTCTCAACCACCAAGAAGCAAACTTTACTAA |
| | | TCCCTGAGTGTCAGGTTTCTCATCTCCCACATCCTATTTTCAT |
| | | TTGCTCCATGTTCTCATCTCCGTTAGCACAGGTCACTGGGGG |
| | | TAGCCCTGTATAGTTTCTAGAAACACCTGTATCCTCTGGGGA |
| | | AGCAGTCATTCCTGGCAGGAAGGAGAGGCTGTCCCTGTTTTG |
| | | AACCTCCCCATGATGTCACAGGTCAGGGTCACCCCCTCTCCC |
| | | AGGGCTCCAGACCCTGCCTCTGGGTCTGAGACTGTGTTTCTG |
| | | GTGCTGTTGATCTGAGTTATTTGTTGTGATCTGGGAAGAGGA |
| | | GAAGTGTAGGGGCCTTCCTGACATGAGGGGAGTCCAATCTC |
| | | AGCTCTGCCTTTTATTAGTTCTGTCACTCTAGACAAACTATTT |
| | | AACCTCATTGAGTCTCAGGCTTTCTGTTTATCAGATGTTGAA |
| | | GCCGTGCCTTACATCAGGGCTGTAATATTAGAATGAATTTGA |
| | | TCCCTGAAACTTGTAACTGTTCAGTGTGATTTGAAAACCTTT |
| | | TTTTTCTCCAGAAATGGCTAGTTATTTTAGTTCTTACAGAGCA |
| | | GCCTTCTTCCCCATTTTCAAAGCTATGAATATTGCAGGGTCT |
| | | CAATTAAAAAGGTTCAATTTGGGATAAAAATCACTAAACCT |
| | | GGCTTCCTCTCCCAGGAGCACAGTCTGAATCTGCACAGAGCA |
| | | AGATGCTGAGTGGAATCGGGGGCTTTGTGCTGGGCCTGCTCT |
| | | TCCTTGGGGCCGGGCTATTCATCTACTTCAAGAATCAGAAAG |
| | | GTGAGGAGCCTTTGGTAGCGGCTCTCTCCATAGACTTTTCCA |
| | | GAGGAGGAAATAGGGCTTTGCTGAGGTTAGTTCTCAGTATAT |
| | | GAGTGGCCCTGGATAAAGCCTTTCTTTCCACAAATGACCTCC |
| | | AATGCCCTGCTAATCCAGAAATCATTAATGCATGGTTACTAT |
| | | GTCAAAAGCATAATAGCTTGTGGCCTGCAGAGATAAGAGAA |
| | | AGGTTAACTAGTTAAGGGTCCTTTGGTTTGAAATCCTGGAGC |
| | | AAATTAAGGAAGAGCCACTAAGGCTAATGCAATTACACTGG |
| | | ATCCTGTGACAGACATGTCATGCTTCATGGGTCACATGGTCT |
| | | GTTTCTGCTCCTCTCTGCCCTGGTTGGTGTGGGTTGTGGTGTT |
| | | AGAGAAATCTCAGGTGGGAGATCTGGGACTAGGACATTGTG |
| | | TTGGGAGAATAGATTTGCTTCCATACCTTTTAAGTGTATATC |
| | | TTTTCCTCTTTTTCCCAGGGCACTCTGGACTTCACCCAACAGG |
| | | TAATACCTTTTAATCCTCTTTTAGAAACAGATTCAGTTTTCCT |
| | | AGAATGATGGCAGAGGTGATAAGGCATGAGACAGAAATAG |
| | | CAGGAAAGACTTTGGATCCAAATTCCTGATCAGGCAATTTAT |
| | | ACCAAAACTCCTCCTCTCCACTTAGGCCTGTGCTCTGCAGGA |
| | | GTATTGGTTCAGGGAGACTTAGGAACTTGTTTTTCTTCTTCCT |
| | | GCAGTGCTCTCATCTGAGTCCTTGAAAGAGAGGAAAAGAAG |
| | | CTGTTAGTGGAACCAGGTCTGAAAACAACACTTTCCTCTCTC |
| | | TCTGCAGGACTCGTGAGCTGAAGTGCAGATGACCACATTCA |
| | | AGGGGGAACCTTCTGCCCCAGCTTTGCATGATGAAAAGCTTT |
| | | CCTGCTTGGCTCTTATTCTTCCACAAGAGAGGACTTTCTCAG |
| | | GCCCTGGTTGCTACCGGTTCAGCAACTCTGCAGAAAATGTCC |
| | | ATCCTTGTGGCTTCCTCAGCTCCTGCCCTTGGCCTGAAGTCCC |
| | | AGCATTGATGGCAGTGCCTCATCTTCAACTTTAGTGCTCCCC |
| | | TTTACCTA |
| 36 | CIITA | ATTCACCATATCCGTTTGTTAGCCCCACTAAGCAAGGCTGTT |
| | | TCAAATTGACTCAGCATTTCTTATTTGTCAATTTCTACCCCAC |
| | | TCCTCACCCGGGGACTTCTGAAAACAGTGAAGGCTTCAAAG |
| | | TAATCACCTTACAAAGAAGGGCATCATCCTCATTTCACACAT |
| | | GGGGAAACTGAGGACCACAGAGAGGAAGTAAAGAGGCCAA |
| | | GGTCACACACCTGCTACTAAGTTCCCCGTCCAGTGCTCTTTC |
| | | AGCAAAAATGCATGAGGCACACAAGTCATGTTTCCAAACCT |
| | | TCATTTCAGTACCACCTTCATCATTTTTGTGTTTTCCACATAA |
| | | CACTTTACTATTATTTATGACAGGGTTTTTTTTAACCACTCAC |
| | | CTTTTTTACTTATCTTTCTTTTCTTTTTCTTTAGGAGAGGCAG |
| | | GATCTCACTCTGTTGCCCAGGCTGGAGTGTATTTCATGATCA |
| | | TAGCTCACTGCAGCCTCCAACTCCTGGGCACACTCGATCCTC |
| | | CCACTTCAGCCTCCAGAGTAGCTGGGACTATAGTTGTGCACC |
| | | ATCATACATGGCTAATTTTTAAAAAATCATTTGTAGAAAAAT |
| | | TAGCTGGATGTAGGAGAATGGCGTGAACCCAGGAGGCGGAG |
| | | CTTGCAGTGAGCCAAGATAGCGCCACTGCAGTCCAGCCTGG |
| | | GCGAAAGAGCGCGACTCCGTCTCAAAAAAAGAAAAAAAGA |
| | | AAAGAAAAGAAAAATTAGCTGGACATAGTGGCAGGTGCCTG |
| | | TAATCCCAGCTGCTCGGGAGGCTGAGGCAGGAGAATCAGTT |
| | | GAACCCAGAACCCGGGAGGCGGAGGTTGCAGCGAGCCAAG |
| | | ATCATGCCATTGCACTCTAGCCTGGCAATAAGAGTGAAACTC |
| | | CGTCTAAAAAAAAACAAAAACAAACAAAAAAAACCCCAAC |
| | | AATTTGTAGACATAGGGTGTCACTATGTTGCCCAGGCTAGCC |
| | | TCCAACTCCCGGCTTCAAGCAATCCTCCTGCTTCGGCCTCCC |
| | | AAAATGTTGGAATTACAGGCACAAGCCACCTGGCCCAGCCA |
| | | TCTACTTTATATTCAAATAAAACTTTACGTCCCATTATAAAG |
| | | GGAAAAAATGGCAAAAACAGGAGGTAACCATTTAACAAGA |
| | | AAGCAGAGTGATGTTAGATTATAGCAAGATACTGTTGACTGT |
| | | AGAAGGCTCTGAGGCTAGAGAGCTGCTTTCTATAAAACGGA |
| | | GTGATCATATATTAGAAGAGGTGTTAAAGACATGTTCACACC |
| | | AAGCTGAGACTTCCTCCTTGATACCACCAGGAGGATGGGCA |
| | | GAGACTGGAAAAGACACTAACTTTCTCCCTATGGGAGTCAG |
| | | TATTATTTAGCATCACTTTGGCGGGTCACCCCAAACCATCTG |
| | | ACTACAAGGGTACCATATTTGGGTTAACACTCTTTTGGTATA |
| | | ATTTATGTTTTAGTCCAATGTCTTGGGATGAAAATGACAGGT |
| | | GGGCCACTTATGATCTCCAGAGAAATTCAGGGCAATTTGGTG |
| | | TGGGAGTAGGCATGGTAGAGGAGAGCAGCATCTAAGAAGTC |
| | | CCCAGCAGAGGCTCTCAGCTTGTCTTGAGGCATCTGGGCGGA |
| | | GGGCTATGATACTGGCCCCATCCTGCAGAAGGTGGCAGATA |
| | | TTGGCAGCTGGCACCAGTGCGGTTCCATTGTGATCATCATTT |
| | | CTGAACGTCAGACTGTTGAAGGTTCCCCCAACAGACTTTCTG |
| | | TGCAACTTTCTGTCTTCACCAAATTCAGTCCACAGTAAGGAA |
| | | GTGAAATTAATTTCAGAGGTGTGGGGAGGGCTTAAGGGAGT |
| | | GTGGTAAAATTAGAGGGTGTTCAGAAACAGAAATCTGACCG |
| | | CTTGGGGCCACCTTGCAGGGAGAGTTTTTTTGATGATCCCTC |
| | | ACTTGTTTCTTTGCATGTTGGCTTAGCTTGGCGGGCTCCCAAC |
| | | TGGTGACTGGTTAGTGATGAGGCTAGTGATGAGGCTGTGTGC |
| | | TTCTGAGCTGGGCATCCGAAGGCATCCTTGGGGAAGCTGAG |
| | | GGCACGAGGAGGGGCTGCCAGACTCCGGGAGCTGCTGCCTG |
| | | GCTGGGATTCCTACACAATGCGTTGCCTGGCTCCACGCCCTG |
| | | CTGGGTCCTACCTGTCAGAGCCCCAAGGTAAAAAGGCCGGG |
| | | AAAGCATCTTAATTTAGCGTGCAGTCTCAGCTGGTCCTGCCA |
| | | TTCCAGATAAACAGAGAAACCATTCTGAATTGGGGATGGGG |
| | | GTGAGGATGGGAACAGGAGTCTGTGTCCTGCTGGGGCAGGC |
| | | CATTGGAAGATGTGAAAGAGTTGTCTATTTCCTTCCACCGGA |
| | | GGGAGACTTCAGGTCAGCCAGGTGTCTGGAGTATGAACCAT |
| | | GTATCAGCACCGAAAGGTTCTAGAAGTCAGACTTTCGGGCA |
| | | GTGTGTCACTAACTCTCAGCATGCTGGCCTGGCTCGGCCCAC |
| | | AGCAAGGTCTTCTCGCCTCCCTTTGGGTAAATACTGAGGG |
| | | GTGCCTCTGCAGGACGGGACCTCTGCCAGACTCCACTCCATA |
| | | CCCAGAGAAGCAGGGAAACCAAAATTGGAGTCAGCCTTGAG |
| | | GTGTAGCTGTTGAGCCCTCAGCAGCTGGGGAGAGCTGGCGG |
| | | ATGCTGCCCTCCCCCCAGTTTCCTAATGGTGTTGTTTAAAAA |
| | | GGGTCAGGGGACGGGGGAACAGATGGTGGGAAGAGCACAG |
| | | TGCAGACACCTGGCACCGGCTCTGAAGGCAGCATGGCAGCT |
| | | ACACCGTTGGCTGGGAAGGGTGTGCCCCTGAAGAAGTCGTT |
| | | TACATTCTCGAGTCAATTTTCCTGGAGTGTACAATGGACCTG |
| | | TGGGAAAGCCTGTATGAAAGGGTAATGATGAGGGACCTAGC |
| | | ACAGTGTCCAATATTTTATAGGAACTGGAATTGAGCTCATAG |
| | | GAGCTCAATTTTATTGGCATTGCTGTTGTTGGATGGTTAAAG |
| | | GGGTGGTATCCCTTTTCTCAGACTCCCCTGAAATGTATGGTT |
| | | TGCTTTGAACCCAGAGACTGATGACAGGTCTGCCGGTGTGGT |
| | | TGGGTGCAGCCTTAAGTTGCTACGGGAAAGTGTTGGAGGGG |
| | | GAGAAGTCAGAGGTAACCTTGCCCCCTCCCTCAATTCCAGAT |
| | | GAGGAAATTCAGGCCTGAAAAGGGAAAGTGACCACCTCAAA |
| | | GTCTCATGCCTTGGAGGACCCAGCAGGAATCCAAGACCTCT |
| | | GAAAAGGACCGGCAGGGCTCTTGCCACGGCTGGGGGTGTGG |
| | | TCATGGTAACACAGGTTTTCCATCCATGGAAGGTACCTGAGG |
| | | GATTTTCTCTTCCTCCCTAGGGCCAGCATCAGAGGAGTGAAT |
| | | AGCTCAGTTAGCTCATCTCAGGGGCCATGTGCCCTCGGAGGT |
| | | GGTTTGCCACTTTCACGGTTGGACTGAGTTGGAGAGAAACA |
| | | GAGACCCACCCAGGGGTGGGGACAAGCTCCCTGCAACTCAG |
| | | GACTTGCAGATCACTTGCCCAAGTGGCTCCCTAGCTCCTGGC |
| | | TCCTGGCCCGGGGCCTGGGACTCTCCCCGAAGTGGGGCTGG |
| | | CCACTGTGAGGAACCGACTGGAGGCAGGGACCTCTTGGATG |
| | | CCCCAGGCAGTTGGGATGCCACTTCTGATAAAGCACGTGGT |
| | | GGCCACAGTAGGTGCTTGGTTGCTCCACAGCCTGGCCCGAGC |
| | | TCAGCGCTGCAGAAAGAAAGTGAAAGGGAAAAAGAACTGC |
| | | GGGGAGGCGGGGAGGTAGGATGACCAGCGGACGAGCTGCC |
| | | ACAGACTTGCCGCGGCCCCAGAGCTGGCGGGAGGGAGAGGC |
| | | CACCAGCAGCGCGCGCGGGAGCCCGGGGAACAGCGGTAGGT |
| | | GACCAAAGTCTCCTCTGTAACCCCTAAGGTCGGGCTGAGAAT |
| | | CGAGGCTCCGAGACTGTCAGCTACTTGCTCAAGGTCACACA |
| | | GCAAGTCTGGGAGGATGGGGGGATGGAATATGCAAAATGTA |
| | | GGGCCGGGAAACACCTCGTTTCCAGCATCCCCGCAACGACT |
| | | CTGCGCGGGAACCAGGAGCCGGGAACCCGGAGCTTGGCTTG |
| | | CTGTGCCCAGAGCTCCGGGGCCGTGGGCGGGTGGCAGGAAA |
| | | GCCTGGCGGCAGCTTCTGCAGAGAAGCCGGAGCGCAGACTG |
| | | GGAGCGCGGAGCAGACACACTCCCCCGGCCACCCTTGGCCG |
| | | ACTCCGCGCGCCCGGGATCCTGCAGAGGTGCGCGCCCTTCTT |
| | | GTACGCCAGACTTTGGACCAGGGCCGCCGTTCCCTGAGCTTC |
| | | ACTTTCCCTGTTGGGTCATATTCCATCTCTAACTCTGGAATCT |
| | | TGGGTATTGGGCTCTCCAGGCGGGGGGCCCTGCTCAGGGAG |
| | | GCAGTAGGGAGCCAAACCTTTAACCAGAGGATGGGATAAGT |
| | | CCTCAACTCTCGTTGAACATCTTGGCGAAGGTGTGTGTTGTT |
| | | GGGAGGGGTGGGGGAGGGATCCCCCCGGACTGAACCGATCT |
| | | CTTGATCTCTCACTTCTCTACCTCGCTTTGGGGCCCTGAGTCA |
| | | CACCCTCTAAGGAGAGAGGCTAAAGCGCCCCGGAAAGCCAG |
| | | CGTGCGAATGCCGGGGTGGGAGTGGGAGATTGGATCTCCCT |
| | | GGGGTCCAGGAAAGCCGGAATCGGAGCCACCATGCTTAGCT |
| | | TAGTCTGGAACTCTTAAAAGCCGCGGTCCTCCTGAGTCCCAC |
| | | AGCCCCTCTCCACCCTAGGTGGCACAGGAGAGGTGGCAAAA |
| | | GCCTAGAAGTTCAAGGCATGGCTCCCTCCCCAGCCGCAGCCT |
| | | GGAGTGTCTAACTTTGGCAGGAAGTCTTCCGTTTCTGCTCCC |
| | | CACTCCAGAGAAAAAATAAATAAATACTTCTCCGGAGTGAG |
| | | ATTAAGGAAACAGGTACTTCTTCCTCTTGGAGAAAGAGGAG |
| | | CCAAAGGAACTTGACTCCAACAAATGATCACCTTGCAAACC |
| | | CCCGGCTCCCTTAGGGGATGACCTGGTCTCCAACAATCTCAG |
| | | AGCGTTTGGAGGCAGGGTCTTTGGAGATGACTGAGTGGGGA |
| | | ATCCCAGGCTCCCCACACATGAACATCACCTGGGATGATCA |
| | | ACCTGTTCAGGATGTAGGTTCCCGGGCTCACCCCCAGGCCCG |
| | | GTTGGCTAGGCCTGGGGTGAGGCTGAGATCCTGCAGGTTAA |
| | | ACCATCTATCCCAGGTGACTCCAATGTTCGTTTGTGGGGCAA |
| | | AAGTCCCTCAAGTCAGAGACACTGGGAGGCGCTGATGTGGT |
| | | CTCATCTCTTTACTCTCTCCCTGTTACAAAACCTCTATCAGAA |
| | | AAGGAGTACCAGGAGGTGTTTTGTTTTGTTTTGCTTAACGCC |
| | | ACATAGCAAGCAGTTTGCAGACGCAGGATTTGAACCCTGGT |
| | | CTACTGAGAGCCCAGCCCAGTGCAGCAAGCAGATGTGAACC |
| | | TCCACAAATGCAGGGCAGGTCCCAGGCAGTCAGAGAGAAAC |
| | | CTGTTGATACCCAGTCTGTCTGATGGCTGGGGCTTGGTTTCA |
| | | TGTCCTAAGACCCAGCCTCAGATACCTTGAGAAGAGCAGAA |
| | | TCAGAAATTAACCCAAGGAAACTCTGGATTAGTTCCAGGCA |
| | | ATCAGGGAAGTGGCTGGAGGGAGGTAGGAAACTTCTGGACT |
| | | TAGCCTCAGATTTGCCTTGAGTCACACTGACAAGTGACTTCC |
| | | CCTCTCTGAGGTTCATCTGTAAAATGGATAAAACATGAGAGC |
| | | TTGCTTGATGATGTCTGATGTGTACAGAAAAAGCACTTTAGA |
| | | AAACCACCAAAGAATATTCATGCATTGGGAATAATTATTATA |
| | | GATCCTAAAGGGATTGTTTAGAAAGATCAGAGAGGGGCCGG |
| | | GAGCAGTGGTTCACACCTGTAATCCTAGCACTTTGGGAGGCC |
| | | GAGGCGGGTGGATTGCTTGAGGCCAGGAGTTCAAGACCAGC |
| | | CTGGGCAACCTGGAGAAACCCCAACTCTACTAAAACACTTTT |
| | | TTGTGTTTTAGCCGGGCATGGTGGTGCACACCTGTAATCTCA |
| | | ACTACTTGGGAGGCTGAGGCACGAGAATCACTTGAACCCAA |
| | | GAGGCGGAGTTTACCCGAGAGGTGGAGGTTACCCGAGAGCT |
| | | GAGATTGCACCACTGCACGCCAGCCTGGGCAACAGAGCAAG |
| | | ACTGTCTCAGGAAAAAAAAAAGAGAGAGAAAAAAGTCAGA |
| | | GAGGAAAGCATAGGCAGATGGGAGGTTTCCAAAGACCAAG |
| | | ACACACACAGTCAAAAGACAGTTCAGAAAGGGGCATTAGTT |
| | | ACCACCGATTCTCATAGGAAGGACATAGATAGTTTTCAGATT |
| | | CCTTCAATCCTTCTGACATCAAGGACAAAGTCTCTGATTTTC |
| | | CCTGATGTTTGACAGTGCCCTAATGCTTGCTAAGATTTCTTTT |
| | | TAACAAAGTGGTAAAGAAGCTGAGATTATTTTAGTAACATT |
| | | GTTTCTTTTTCAACCACAAGTGATACTGTTGGTTTGGTTTTGC |
| | | TTTTCCGTTTATACATGGAAAAAAAAGTTTTCTTTTTGTAAA |
| | | ATCATGGTAAAATACATATAACATAAAATTTACCATTTTAAC |
| | | CACTTTCAAATGTACATTTCAGTGGCATTAAATATTTCAAAC |
| | | TGTTGTGCAACCTCCGCTACCATCCATCTCCAGAACTTTTTCA |
| | | TCCTCCCAAACTGAAACTCTACCCAATAAGCAATAACTCCCC |
| | | ACTGCCCCTTCCACCCAGCTCGTGGTAACTCCAACTTCCTGT |
| | | CTCTATGTTTTGGGCTACTCTAGACACCGCCTATAAATAGAA |
| | | TTACGTGATATTTGTCTTTTTGTGCCTGGCTTCTTTCACTTAG |
| | | CATAATGTCTTCCAGGTTCATCCATGTCGTGCCATGTGTAAT |
| | | TCCTTTCTATGGCGGAATAGTATTCTAATATGTGGATATCCC |
| | | ATATTTTGTTCATCCATTCATTTGTTGATAGACACTTGGGTTG |
| | | TTCCTACCTCTTGACTATTGTGAATAATCTTGCTAGGTACATG |
| | | GGTGGACAAATATCTGTCTGACTCCCCGCTTTCAACTCTAGA |
| | | AAGCTTTCTTTTAAAATCGGTCTATTTAAGTTTCTTTAAAAAG |
| | | TTAATTTAAGAAAAATACTAAGGTAAATTGTAGAACAGGTG |
| | | ATATGTCAATATGACCCCAAAAATTGTAAAGATGGAAGACG |
| | | AATGCCTGCAGTTTGGGAAACCGGATGAGAGGAAAGACCTG |
| | | GAAGCTATTGTAAAATCTATGCTGAGGGTCTGGTGACTACTA |
| | | GACCAAGGGCATGGCAGCAGAGGGCAGCTGAAAGACTTCAG |
| | | GAAGGAAAGATATAAGAATTAAGAAAGGGGCCTGGTGCAGT |
| | | GGCTCATGCCTGTAATTCCATCACCTTGGGAGGTCAAGGCAG |
| | | GATGATGGCTTGAGACCAGGAGTTCAAGACCAGCCTGGGCA |
| | | ACAGAGCAAGACCTTTTTTTAGGAAAAAAATATATAAATTA |
| | | ATAAAAATAAATTTTTGAAAATACAGATTCCAGGCCGGGCA |
| | | TGGTGGCTCACACCTGTAACCCCAGCACTTTGGGAGGCTGAG |
| | | GCCGGTGGATCACCTGGGATCAGGAGTTCGAGACCAGCCTG |
| | | GGCAACATGGTGAAACACCGTCTCTCCTAAAAATACAAAAT |
| | | TAGCCGGGTGTAGTGGTGCATGCCTGTAATCCCAGCTACTCG |
| | | GTAGGCTGGTGGAGGAGAATCGCCTGAACCCAGGAGGCGGA |
| | | GATTGCGGTGAGCCGAGATCACACCATTGCACTCCAGCCTG |
| | | GGCAACAAGAGTGAAACTCCGTCTCAAAAAAAATGAAAGAA |
| | | AATACAGATTCCTGGGCCCGGCTGCAGACTCCCTGAATAGA |
| | | ATCACTGGGGGTGAGGCCCAGGACGCTGTGTTTTTAACACCC |
| | | TTCCCAGGTAATTCTCACTCACAGCCAAGCCTGCCGATGGCT |
| | | GGACTCCGTAGTCAGATCTCAGCAAGCAGGACTGGCTCGTG |
| | | GTTCATCTTGGGGTCTCCAGCACCCAGCAGAGTGCTGGCGGG |
| | | GGGACTGTGTTAAGTAGAGCCGGATGGGTCAGGACTGGAGT |
| | | GTGGGGCCTGGCTTTGCTAACTGCAGTCACAAGATCCTCATC |
| | | ACTCCACCCCCTACCCCAGCACCAGGGTCAGGTTCCCTTCCA |
| | | GGAAAAACTTGTTGGACTCCTTTCCAGGTCATTGACTGCAGT |
| | | TAGGAGTGGCATGCAGAAGGGAGGCTATCTGGAGCTGGGAC |
| | | AGACCAGGCAAGCGGGGAAGGGAACTCTTATCCCAACCCTG |
| | | CATTCTGGACAGGGACCGAAGGTCTCCTGGCTGCAGCTCATA |
| | | CAGCTCATCTTCATTCTCATCCTGGGACCACCAGGCCCCTCA |
| | | CCCACAATAACTTTACCCTTTACTGGCAAATGCACAAGCTGG |
| | | AGATGATGGCCCATTCGAGTTTTAAGATTTTCCAAGTTGTTA |
| | | TGAGGTCAGTGTCCTCAATCGGGACCCAGAGAAGATAGGTG |
| | | GCTTGCCTGAACTTTCAGAGGGGGCATCTGTGGTGTCTTTTG |
| | | TGGGGTCCTGTAGGATTTCCACGTCCCTCTTGGAGGAAAAAT |
| | | AGAAGTAAGTTGTCTCAGGTCAGATTGTCTGGGAAGCCAAG |
| | | TAGGAGATGGAGATTTGCATATAGGAGGTTTACTGGGGACA |
| | | TTCACAGGAGGGAACACCCGAAGGAAAAGGGAAGAAAGCA |
| | | GGGTTGGGAAGCAGCAGGAGTTGAGGTGCAATGCAGTCACA |
| | | ACAGAGGTCTTAGCCAATCCCATAGGGAGCACTCAAGCTGG |
| | | GACAGCAAGGAAGCTAGACCTTCACATCCCTCATCTACCTCC |
| | | GTCCTCCCATACAGGCATAGCCTCCCCCATGATGAACACTCA |
| | | GCACCAGACTGATACATTTGTTATAATCAATGAACCTACATT |
| | | GATGCATCATTATTACCCAAGGTCCATGGTTTGTTTACATGA |
| | | GGGTTTACTCTTGGTGTTGTACATTCTGTGGGTTTGGACAGA |
| | | TACATGATGTAATGTATATGACATTATAGTATCACACAGACT |
| | | CATTTCACTTGTGACTGAGCTTGTCCTTCTTTCTTTAACCTGC |
| | | CTCCAACTGCTGAGAAGCCCTTTGGCTAGGGATTGCAGCCAA |
| | | TTTTAATTTGTTTTCCACTTTCCTGCTCTGAAGTTACTTTGTTT |
| | | GCCAGAGGCTGGTTGGTTCAAACCCAAAGGGCAGGGAGCTG |
| | | CAATGGAAATCCATTCAACGGTGCTGGAATTGGGATGGCAA |
| | | AAAGGAGCAGCAGGCAGGTGTCAGCAGGGAGCAGCTCTTTG |
| | | CATCCCACAGTTCCCAGGGAGCTTACCATTCTTTCATTCATTC |
| | | ATTCATCCTTTCATTTATGAGACAAAGTCTCACTCTGTTGCCC |
| | | AGGTTGGAGTGCAGTGGCACAGTCATTGCTCACTGCAGCCTC |
| | | AAGCTCCTGAGCTCAAGCCATCCTCCTGCCTCAGCCTCCCAA |
| | | GTAGCCGAGACTACAAGGGCACTCCACCACACCCAGCTAAT |
| | | TTTTGTATCTTTTATTTCTGAAGAGATGGGGTTTCACTATGTT |
| | | GCCCAGGCTGGTCTCAAACTCCTGGCCTCAAGTCATCCTCCC |
| | | TCCTCAGCCTCCCAAAGTGCTGGGATTACAGGTGTGATCTCA |
| | | GACAGCTTACCTCTTAATCACCTTTCCATCTGAGCTGGCAGC |
| | | AGAGCTGGGAAAGTGCACCCAACACCCCAGATGCTGCAACT |
| | | TTAGTGCTTCCTGCTCCTTCTCTGTGTGGAACTGGCCTGTTTG |
| | | GGTACCCAGGGCCAGTCCCCTCCAAGACCCTCTGTAGAAAA |
| | | AGACCCTACTGTACACCTCAATTCCATTGCAGGACAGGACAT |
| | | AATTGAATCCTGCCTCTCCCTGCCCCAGTCACCCATCCCTCC |
| | | CAAGCCATTATCTTGGGACCTGCAAGGGCCTCCAAGCCAAG |
| | | GGTGGCAAATCCAAATACCAAAGGGGCCATAATGATGTAAA |
| | | ATGTGTCCAGGCAGAAGGCAATAGGGAGTAGTGGGGCCTCT |
| | | GGTAACAATTGTGTGTACTCTATATAGAGACATTGTGATGGT |
| | | GATTTAGACACACACACACTTATATTGGCTTGGGAAACAAA |
| | | ACAAATAGGCCTATCGACTGGATTCAACTTTTGATTTTGCAG |
| | | CCAAGCCCCTGTGGCACAGCTGAGGAGCTTGCAGGTCAGAA |
| | | AGGGGATTTAGAGCCAACCAATAGCAATATCTCCAGACCTC |
| | | TCCTGACACGAAGCTCATCTGACCTCCTGCCCCCATTACACA |
| | | CATACACATGCACACACATGCATGCAGGCACATGCACGCAC |
| | | GTCTTCTAGTCTCCCTGCCTTGAGGACAAATGATCAGATCCT |
| | | GCAACTGTTTCCAGGCCCATAAGCCCCTGAGCATTGCAGTGT |
| | | TGCTACATGCCTTAGTTTCCTCTCCGTCTCCTGTGACCTCCTC |
| | | GTGCCACTCCAGGAAGTGAAAGTCTTGAGTGAGACCAGTAG |
| | | TTTCTCTTAAGTCCTCTCTCTGTCTCGTTCTCCTGGGATATCT |
| | | ACACTTGGTTTGCAAACAGCAGGCTGGAGAGTGAGGGGCTT |
| | | CTTCCTCTACATCCAGCTCCTCCCCCATCCTGCTCCAAGTCCA |
| | | GCATGTTCCTATCTCTTCCCCAGGACCCTGGGCTCTTGGGCA |
| | | CAAATCAAGCCATCACCAGACAGCCTGTGGGGTGGCAAGAG |
| | | CAGAGGTTTGTTTCTTTGTGAACATCTATAGGTACCAGGCTC |
| | | TGTGCAGGCCCTATTGTTGGTGAGCCATAGAGGTACCAACCT |
| | | CACCCCATGAATCCTCCTTTGTTTCTTTTCTTTTCAACCCCTG |
| | | GGCCTTTAAACCTTCGATTAACTGCCCAGTGCAGCCCAGGTC |
| | | ATGGTACACTATGATCTTTGGTTCACATGAGGGTTTTCTCTTC |
| | | ATTAATGTCCCTATTTCCCATACTGGGTGCCCATTCTAATGC |
| | | ATTTCAGGTGTATCTTTCTGTTTGTATGTGCTCTTATAAAACA |
| | | TGTTTTGCCTTTTTTTTTTTTTTTTAGATGGAGTCTCACTCTGT |
| | | CACCCAGGCTGAGTTTTCTATTTTTAGTAGAGATGGGGTTTT |
| | | ACCACCTTGGCCAGGCTGGTCTCAAACTCCTGACCTCAGGTG |
| | | ATCCACCCGCCTCGGCCTCCCAAAGTGCTGGAATTACAGGTG |
| | | CGAGTCACCACGCCTGGCCACAGTTTCTTACTTTTGCATTCT |
| | | GTACTATGTTTTTAGGATCTACTTGCCTTGCTTCTGATGCATT |
| | | TGATCTTTGCTTCTAATCACGGACATTGTTCTCTATGGAGTGC |
| | | AGGGGTTACTTTTCACCTGTCCACACCCCCAGTGATGGACAC |
| | | CTAGGTGACCTCCAACTCTCTGTCACCACAAATAACACAGCA |
| | | GTAACCATCCTCAGATGGGTTCTCCTGGGGAGTIGTGGGAAC |
| | | ATTTCTTCAGGATATATAGACCCAAGAGCTGAATTGCCAGGT |
| | | GGTAGGATCTACATATCCTTAGATATTCTACATGATGCCAGG |
| | | TCGTTCTCCAGAGCGGCCCCATCCATCTACATTCCCACAAAC |
| | | CCGTGTAACCAACACTTGGCATTTCCCAGATGTCTAACTTTT |
| | | GCCAGTTGTACAGTATCAAGTGTCACTTTGCTGTTTTATTTTT |
| | | GGAGTTTACATCTTAGTACCACTTACTCGCTAAGTCACTTTA |
| | | GGTAAGTCACAACAACCCAGTGACATAAGCACTGTGCCAAC |
| | | AAAATGCAGTCGATTCTGTGACTCAGAGAGGTTGAGTAACA |
| | | TTTGCAAAGTCACACAGCAAGTGTGGAACTAAGATTTGAAC |
| | | CCAGAGCCTGCACCCTTAACCACTATGCTATTATTATGTTTT |
| | | GTCTCTCTCCTAAAATATCATCTGGGTCTACATTGCAAGCCT |
| | | CTTTGGCAGGGACTGTGTGGTAAACCTCTTTGGGGCCAGTAA |
| | | GGAACTGAGCTCAGTGACTTTCTTACGGGGTGCTCAGTGAAT |
| | | CATTGTTTTACAGCTTTAGAGGGGAAAAGTGAGAGATGTTTC |
| | | TTAGAGTTTATCTGGAGGACTACAGGGTGCATGAGCAAAAG |
| | | CGGAACTCATTTGAGCTACTACCCCCACAAAGAAGCTGTTTG |
| | | GGGTCTGGTCATGGTAGATAGGCATTCCTAGGCCCCTGGCTT |
| | | CCCAACACCATTTGGCCCCCGGAAGCAGCTTGTCATGAGGTT |
| | | GGGGAGCAGGTTGGTGTTGGGGGGGGCCTTGGGGGAAAGGA |
| | | AAAGGAGAAGACTGCCAGGCAACGAGGAATCAGAATCTCA |
| | | AAAAGACTCAATTGAAGGGGCTCCAACACTCGTTGAAACGG |
| | | AGGCTCAGAGGGCTAAGGTAATGTACTCAGGATCAAAGGCA |
| | | GAGCAGAAACTCAAACCATGTCTCTAGGCACACAGTTCAGA |
| | | GGTCTTCCCATTATTCCTCAGGAGGAGAAGTTCATGGTCAAG |
| | | AGTAGGGAAGTATTCCTTCTCAAGCTTTTTTTTTTTTTGTCTG |
| | | AGACAGAGTCTTGCTCTGTCACCGAGGCTGGAGTGCAGTGG |
| | | CGTGATCTCGGCTCACTGCAACCTCCGCCTCCCAGGTTCAAG |
| | | CAATTTTCCTGCCTCAGCCTCCCAAGTAGCTGGGATTATAGG |
| | | CACCCACCACCACGCCCAGCTCATTTTTGTATTTTTGCAGAG |
| | | ACGGGGTTTCACCATGCTGGTCAGGCTGGTCTTGAACTCCTG |
| | | ACCTCAAGTAATCCGCCAGCCTCGGCCTCTCAAAGTGCTAGG |
| | | ATTATAGGCGTGAGCCACCACCCCTGGCCTCCTTCTCATGCT |
| | | CTTTAATCACAGTTCAGAGCAGTGCTGGAGGCTTTTAGGGGT |
| | | AGCAAAAGAGCCTTCTTCTCCCTTTCAGTGCCTCCTTAGACC |
| | | CTCAAAGATAGACTCCCCGCCTCTGGGAAGAAAACATCTAA |
| | | TGAACTGGACGTAATCTCAGCGCCTGCGTCTGTAAAATGGCT |
| | | ATAATAACAACAATCTCCCAGAGTTGCCGTTAACAGTTGAA |
| | | ACAGAATAGTAAATGCAAGGCATCTAGCACAGCCCCAGACA |
| | | CAGAGAAATCTCCCCTGGTCACTCTCACAGAGGTAAGAAGC |
| | | ACATTAATAATAAAAATGGTCATCACGCATATTGTACTCACC |
| | | ATGCAGAAGTTGTGCTGCCTTAAGCTACTGTTCATTGAGCAC |
| | | TTACTGGTCGCCAGTCCCCTTTCTAAGGGCTCCATGCAACTT |
| | | AACTCCCCTAATCCTATGAGTTGCCAATTATTATCCCCCACTT |
| | | TACAGATAAGGAAACTGAGGCTCCAAGTGGTTTAAAAAATG |
| | | AACTTACCCAGATTGTAGTGCGTTGGTAGGTAGGGATAACCT |
| | | GAAAGTTGGGAAATTTCACCCCAAAGCTCACCATCTGAGCTC |
| | | CATTCATCTCCCACCAGCTGAGATGGAACGTTATGCTGTTCC |
| | | CAGAAGAGATGCATGCACTGATCTTCAGATAGTTTAAGTCTG |
| | | AGTTAGAACATTGTGGCGACACCTGATTTCCCTCGGGGGAG |
| | | AGAGAGGTGAATTGTTATTTCAGACACCTGTTTAGTGTCTAC |
| | | ACTTAGCCTTTCAAATCCTGCATGCAGTGTGAAGTGGGGATG |
| | | AAAATAAACCCCAAAAAGGACTTTTGATGGCAAAAAGATCA |
| | | GGAATATTTTCTCGAGTTGGATGTGGAAGGTGGTGGCCCAG |
| | | GGGGAAGCACAGTTTCAAAGAGGACCTTCTAAAAATACTGC |
| | | CCATTTACCAAAAAGACACAGACCGCGCCTCTGCCCTGCCCC |
| | | CACATGGCTCCTCAAAAGGAGAAATTGTCTTGCCACCCACGC |
| | | CTTCCCTCGCCGGGGTGCTGGGGGGCCAACAGCAATTCAAA |
| | | AAGGCTATAGAGTGGGCTGAGACTTTGGAGATGGGTAGTTT |
| | | GGTTCAAATCTCAGCCCACTAATCCCTCTGAGCCTGTCTCTT |
| | | CATCTGAAAAACAGGAATACAAATAGCTGATGTGTAAGTTA |
| | | GACCAAATGGGGTAATGCCTACGAGTCATGTCATTTGGTACC |
| | | TGGCACTTGCTGGACACTGTGAATTCTAGTTCTATATCATTG |
| | | CCACGGATGAAGAGAGGCTGCCATGCGCAGAAACATGCTGA |
| | | GAGCTGGGAGAAGGTGAGCTAGAGGGATACAGTCCCTGCCC |
| | | TCAGGGAGAGCTGAAGGGCTCTCTCTCCTGCCACGGGATGA |
| | | TCAGGGCCATCTTTTCCCTCCATCCCGCTCTGGCAATGTGGTT |
| | | TGGAGAAACAGTCCAATGAAGGGGCCTTTGAAGCACTTCCT |
| | | AAGCATCTATAGACATAGGAAACTTGGCTCTGGGGATAGAG |
| | | GAAGGTCCCATGCCAAGATTCAAGTAGATCTTGGTGTGACTG |
| | | GGAGGCAGCTGTGGCGTTGTGGGGAAACCTAGAGTCTTGGG |
| | | CCTGAATTTCAGCTTTGACACCCTCTAATGTAATGGGCTTCC |
| | | TTTGACTGTAGGCTTCCCACATAGCTGGCACCCCACACAGAA |
| | | CTCCCTTGGCCTTCTGATGATCCTGCAGGACAATCTTTTTTGT |
| | | TTTTTGTTTTTTGTTTTTTTTTTGAGATGGTATCTCACTTTGTC |
| | | GCCCAGGCTGGAGTGCAGTGGCGCAGTCTTGGTTCACTGCA |
| | | GCCTCTGCCTCCCAGGTTCAAGCGATTCTCCTGCCTCAGCCT |
| | | CCCAAGTAGCTGGGATTACAGAAGCACGCCACCATGCCTGG |
| | | CTAATTTTGTATTTTTAGTAGAGACGGGATTTCACCATGTTG |
| | | CCCGGGCTGGTCTCAAACTCCTGACCTCAAGTGATCTGCCTG |
| | | CCTCGGCCTCCCACAGTGGTGGGCTGCAAGGCAATCTTCATT |
| | | CTCCAGAACTAATATTGGGAAAACTGATGTTCAGAGAGGTC |
| | | AAGTGACTTGGCTAAGGCATCACAGCCAGAATTCAGATGCA |
| | | AGCTCCTAAACCACATGACCTTGGACAAGCCATTTCACTGAG |
| | | GCGTGTGAACCTCCTCTAAGCTCAAATGGGCATAACACTCTA |
| | | CCCTGCCTTTTCCCCAGAGCTTTTTGGGAACTCAGATGGTGT |
| | | GGAGGATGGAGGCAGAAGTCCACTGTGAGCTGCAAATCCTT |
| | | GATCACCCAAGAGGAAGTGCTTAAGGTGCAGGCTTCAGTCT |
| | | TAGGGAAGCAGGAAGTGGGGCGCTGAAATGTGTATTGGGGG |
| | | CTTCTGGGAGGGGGTTTCTTCTTGTCCAGGTTGAGCCAATGA |
| | | CGCAGATCTTAGGCCCTCTGGTGACTTTTTGCCCAGGCAACT |
| | | TCTCAGTGGTGGTGATGGGGAGCGTGGCCAGACCATCCAAG |
| | | CTGCCCTGCTGGCTGTGGGGGCCTTTTTTTTTTTTCTGAGACA |
| | | CAGTCTGGCTCTATCACCCAGGCTGGAGTGCAGTGGTGCAAT |
| | | CTCAGTTCACTGCAGCCTTGACACCTCCCCAGGCTCAAGCTA |
| | | TCCTCCCACCTCAGCCTCCTGAGTAGCTGGTTCTACAGATGC |
| | | ACATCACCACGCCCAGCTAAGTTTTGTGTTATTTGTAGAGAT |
| | | AGGGCTTCACCATGTTGCCCAGGCTGGTCTCAAACTCCTGGA |
| | | CTGAGGTGATACTCCTGCCTTGGACTCCCAAAGTGCTGGAAT |
| | | TTCTACAGATGTGAGCCACCATGCCCGGCCTATGGGGCCCTT |
| | | CTTGTTCCTATGAAGTCTGCAGGGTCCCCTTATGCAAAAGAA |
| | | CAGAAAGCTCGGCTTCACAAAGTTTGACCTCGGAAGAAATG |
| | | GAAGGGAAAGGGTCCCTTTGTCACTCTCACAAACGTAAGAA |
| | | GCAAACTAATAATGATGGCCATCACACATATTATACTCACCA |
| | | TGTAGAAGCTGTGCTGCCTTAAACTTCTGTTCATTCAGCACT |
| | | TACTGATTGCCAGTCCCATTGCTAAGGGCTCCGCGTGACTTA |
| | | ACTCCCCTAATCCTTGTGAGTTGCCAATTATTATTCGCATTTT |
| | | ATAGATATGGAAACTGAGGCTCCAAGTGGTTTAAAAAATGA |
| | | ACTTGCCCAGGTTACAGTGTGTTGGTAGGTAGGGGTAGTCTC |
| | | AAAGTTGGTAAATTTTACGCCAAAGCTCACGATACTTGCCTT |
| | | CTTTCCCTTCTCAATTTATTTCTCTGACTGTGTGCTTAACCAC |
| | | ACAGCAGTGTCATCCAGAGTTTGAAGCAGAGCTCATCAATA |
| | | AGTGGGGGTGGGCTAAATGTCTCCAGGCTCCTCCCTGCGGTC |
| | | CCTCCCACCATCCCCCATCTGTGATACCACCTGTGTCTCTATG |
| | | CTTGAGCCCAGACAGGACAAGGGTGATCATTTCATTGGGAA |
| | | CATTTTGACTGGGGGGAAAGAGCACCAGCCTGGGAGTCTTG |
| | | GCAGGGGGAGTGGGGGAGAAGGGGCAGGGGCAGCCACCGA |
| | | CAGGCTATGAGGTCTTGTGCAAGTCACGTCCTTCCCGGGGTC |
| | | TCAGTCTTGTCATCTGCAGAGTGAAGGAAAGAGGTCAAGCT |
| | | TGAAGGTCCCTGGCAGACCCAATGCCCTGTGAGAAAACTGA |
| | | GTTTGGGTAACTTATTATTTTATAGATATGGAATTATCTTATA |
| | | GAAATGTTATAGATATGGAATCATTTTATAGATATGGCCCAA |
| | | AGTCCAAAGTCACACAACTCATTCACGGAGTTGCTAGGATC |
| | | AGTCTGGAGCTCTTGTCACGAGATCACACTGCCTCAAAGAG |
| | | GAGGCAGATTGCTGCAATGTAGGTTCTTAATAAAGAGCATG |
| | | GGTGGAATGGGAGGCGGGCATCAGGCAGGAGCTGGGGCAA |
| | | AGGTGTCTTTTCTCACTGCTCTGAGAGAACAGATTTTGTGCC |
| | | TGTGTCCCTGACCAAGGGGCTGGGAGAGGTGGAGCATCTCA |
| | | CCCCCAACCATCTCCCAGCTTTGGCCCTGCTCCAGGAAAAGG |
| | | GCGGTGGCCCCATCTTTCAGTTCTGCTTCCTCTAGTCATGGTT |
| | | GGCTGGGATCTCGCTGCAAGCCGGTTATGTTTGGGTTCCTGC |
| | | AGCCCCCGCTCAACCGTCATGAACAAGTGGGCCCAGCCAGT |
| | | GCCCCAGCTTGGACTGGAAAGATCATTCTCTGTGACATCCGG |
| | | GGGTCTGTGCTGAGCCTCTCCCCAGGCAGTGAAAGCTGTGG |
| | | ATGCGTGAAAGAAAATGGGTCTCTGGGCCGGGTGTGGTGGC |
| | | TCACACCTGTAATCCCAGCACTTTGGGAGGCCAAGGCGGGT |
| | | GGATCACCTGAGGTCGGGAGTTCGAAACCAGCCTGACCAAC |
| | | ATGGTGAAACCCCATCTTTACTAAAAATAAAAAAATTAGCC |
| | | AGGCATGGTGGCGGGTGCCTGTAATGCCAGCTACTTGGGAG |
| | | GTTGAAGCAGGAGAATTGCTTGAATCCGGGAGGCAGAGGTT |
| | | GCAGTGAGCCAAGATTGCACCACTGCACTCCAGCCTGGGCG |
| | | ACAGAGTGAGACTCTGTCTCAAAAAAAAAAAGAAAAAGAA |
| | | AATGAGTCTCAGGAATGGGGTGGCAAGTTGAGTGTGTGTTTT |
| | | CTCATGAGCTTACTCTTCAATGTATTCTCTTCTGGGCCCAGA |
| | | ACAGGGCAGTAGGAGCCTCCCTCTGGTCTTCCAGCTTCAGGG |
| | | CCAGCTCTCCTCTTACCTCCTCCAGGAAGCTCCCTCTGTTTCT |
| | | TTCCAGCCTTCATCTGCATGTCATGTGTGGTGGGTTGAAAGG |
| | | TGGCCCCCCAAAAGATATGTCCATGTCAAATCCCTGGAACCT |
| | | GTCAGTGTGACTTTATTTGGGAAAAGGGTCTTTGCAGATAGA |
| | | ATTAAGTTAAGGGGCTGGGTGCAGTGGCTCATGCCTGTAATC |
| | | CCAGCACTTTGGGAAGCTGAGATTGGAGGATCACCTGAGGT |
| | | CAGGAGTTCAAGACCAGCGTGGCCAACATGGCAAAACCCCA |
| | | TTTCTACTAAAAATACAAAAATTAGCTGGTCATGGTGGTGAG |
| | | CACCTGTAATCCCAGCTACTCAGGAGACTGGGGCAGGAGAA |
| | | TCGCTTGAACTGGGAGGTGGAGGTTGCAGTGAGCCGATACA |
| | | GCGCCACTGCATTCCAGCCTGGGCAACAGAGCAAGCCTCTG |
| | | TCTCAAAAGAAGGCAAAAAAAGAATTAAAGATCTTGAAATG |
| | | ACATCATCCTGGATTATCTAGGTGGGTCCTAAATCCAATGAC |
| | | AAGTGTCTTTATAAGAGAAAGAGACTTGCTACATGAGGAGT |
| | | AGGTGATGTGGCCACTAAAGCTGAGTTTAGAGTGATGCAGC |
| | | CACAAGACCACAAGTCAAGGATCACCAGTAGTTCCCAGCAG |
| | | CTAGAGGAGGCAAGGAAGCATTCTCCCCTCGAGCCTCCAGA |
| | | GGCAGCGTGGCCCTGCTGACACCTTGGCTTCAACTTCTGGCC |
| | | TCCAGAACCATGAGAGAATAAAGTGCTGTTGTTCTGAGCTAC |
| | | CTGGATGCTGGTACCTTGTTTCAGCAGCCCTGGGAAATGAAT |
| | | ATACCATGGAAACATGGACCTGAACCACGGATCCTGGCAGG |
| | | AGCCTTAGAGCCACACAGATGAGCCTTCAGCTTGGCTTTCCC |
| | | CAGCAAGAGCTACATGATCTTGAGGAAGCTGCTTGTCTTCTC |
| | | TGGGTCTCAAGTTCTTCATCTGCCAATGGAGACAATAATGCT |
| | | TACTTCCTAGGTTTTAACAGCTTTATTGAGATGTAATTCACAT |
| | | ATACAATTTGCCCACTTAAAAAGTACAGTTCATGCCTGTAAT |
| | | CCCAACACTTTGGGAGGCCGAGGAGACAGGCGGATCACTTG |
| | | AGGTCAGGAGTTTGAGAACAGCCTGGCCAAAATGGTGAAAC |
| | | ACTGTCTCTACTAAAAATGCAAAAATTAGCCGGGTGTGGTG |
| | | GTGGGTGCCTGTAATCCCAGCTGCTCAGGAGGCTGAGGCAG |
| | | AAGAATGGCTTGAACCTGGGAGGCAGACGTTGCAGTGAGCC |
| | | AAGATTGCACCCCTGCACTCCAGCCTGGGTGACAGAGACTC |
| | | CGTCTTAAAAAAAAAAAAAAAAAAAAAAAAAAAAAAGTAG |
| | | AGTTCAGTGTTCTTTTGGGAATACTCAGAGAGTTGTGTGACC |
| | | ATCTCCCATCCCCACAGTCAATTTTAGCACATTTCATCACCC |
| | | CCTAAAGAAACTCCAGACCCATCAGCAGTTGTTCCCCATTTC |
| | | CTTCCAAAATCCCTCTCTGCCAAGCCCTAGGCAACCACTAAT |
| | | ATGCTTTCTTTCTTAAAAAAAAATTAATAGAGATAGGCTTTT |
| | | GCTATGTTGCCCAGGCTGGTCTTGAACACCTGGGCTCAAGCG |
| | | ATTCTCCTGTCTCAGCCTCCCAAAGTGCTGGGATTTCAGGCA |
| | | TGAGCCACCACGCCCCACCCCTCCTACTTTATGTCTCCATGG |
| | | ATTTGCCTGTTTTGGTCATTTCAAATAAATAGAATCATACAA |
| | | TATGTGACCCTTTGTGTCCGGCTTCTTCACTTAGCATTTTTTT |
| | | TAAGGTTCATCCATGTTCCAGCATGTATCAGTGCTTTCTCCCT |
| | | TTAATGGTGGAATACTTTATTGTATAGATAAGCAACATTTTG |
| | | TTTATCCATTTATTTTTTGGTGGATATTTGGGCTGTTTTTATA |
| | | TTTTGGCTATTTTGAATTATCCTTCTGTGAACATTCGTGTATA |
| | | GGTTTTTGTGTGGATATATCTGGTTTTTCTTGTTTTCTTTTGTT |
| | | TTATTAATACATAATATGATACATATTTATGGGGTATATGTG |
| | | AATATTTATTACATGCATAGAAGGTATAATGATCATGTCAGG |
| | | ATATTTGAGGTATCCACATTTGGGATTGTTTAAAGATTAAAT |
| | | GAAATAGTGTTAAAAGTATTTAATATGCCCTTCAACAAATGA |
| | | TGAGGAAATCTTAGAATCTGCTCAGACTCCTTCAGTTTACAT |
| | | ATTAGGAAACTGAGGCACAGAAAGGAGCAGAGACTTGCTCA |
| | | AGTCCACCCAAAGCAGTAGAGCATTGTGGTTAAATGCAGGA |
| | | CTTCAGTCAGACTGTCTGGGTTCAAATCCTGGTTCCACTTGG |
| | | ACATGGGTTTCCTTACATAAATCACTTCACCTCTCTGAGCCT |
| | | CAGTTTTCTCATATGCAAAGTGAGGATAATAATAATACCTTC |
| | | CTTACATGGTTACTGATATGAGTATTAAATGTGCCAGCTCAT |
| | | GTGCCTGGCGTATAGGAGGTGCTTTATAAACCTTAGCTGTTA |
| | | CCACTCATGGCATTGCCAAATGTGGGACGGGTCTCCTGACTC |
| | | TCTGGTGTGAGATTGATGGAATCCACACTTTCCAGTTCCCTT |
| | | TTCTACCTCCTGGGTATCTTCTCATATGGTTGTAAGTTCCTTG |
| | | GAGGAAGGGAATGTGGCTTGCTCTCTCCACCACGCTGAGCA |
| | | TATAAGAGGTGCTGAATGAGCGCTTTTATTCACTCCTCTCAT |
| | | CCCCAGCCCTCACCAGCTGGGAGTTGTTGTAGGTGTCAATTT |
| | | TCTGCCTCTTTCCAACACCCTGTGAGGTGACTGAGCATTGTC |
| | | TTCCCTCCCAGGCAGCTCACAGTGTGCCACCATGGAGTTGGG |
| | | GCCCCTAGAAGGTGGCTACCTGGAGCTTCTTAACAGCGATGC |
| | | TGACCCCCTGTGCCTCTACCACTTCTATGACCAGATGGACCT |
| | | GGCTGGAGAAGAAGAGATTGAGCTCTACTCAGGTGGGCCCT |
| | | CCTCCCTCTGGTCTCTTCCGGTATCCCCCACCCCTCAGCTTGC |
| | | TGTAGAGACGGCAATCAGGGGAAATTCTGGTCCCTGTCCTCC |
| | | CGTCAGCACCACGGACAGCTCCCACGTCTGTGGGACGCTCTC |
| | | TGCAGATGGGGATGATCTCCCAGCCCTGCCCCGCCTCTCCCT |
| | | CGTTCCCCACCAGCCCTCTTTCCAGAAATTTCCTTCTTCATCC |
| | | AAGGGACTTTTCCTCCCAGAACCCGACACAGACACCATCAA |
| | | CTGCGACCAGTTCAGCAGGCTGTTGTGTGACATGGAAGGTG |
| | | ATGAAGAGACCAGGGAGGCTTATGCCAATATCGGTGAGGAA |
| | | GCACCTGAGCCCAGAAAAGGACAATCAAGGGCAAGAGTTCT |
| | | TTGCTGCCACTTGTCAATATCACCCATTCATCATGAGCCACG |
| | | TCAGTCCCCTCCCACAGAAATCATTGCAAGGGGGATGCGGA |
| | | GCAATGGCTGGAGGAACGGAGACTCCAGGGAAGAGAGGGG |
| | | AGATGGAGGCCAGTGGGGGAAATAGGCCCCTTCACTAATGA |
| | | CCACCAAGAAAACAAAATCTCATGTTTACATCCTCCACCTCC |
| | | ATTTCTATACGCATTTCTGCTTCTTGCTCTTCTGTCCATCCTTT |
| | | CTACAAAGCCCATACCATACACCCCTTTCCCTTTTCCTCCCA |
| | | GCTCCTTAGCCAAGCTACTCTAGTATTTGTAATAACTAGCAT |
| | | TTACTGGATACTCATAGTATGCTCATTGCTGTCCATACATTAT |
| | | CATCTGGAATCCTTACAACAACAAAGGAAATACATATTATTA |
| | | GTTTCTGTCTTTTACAAATGAGGAAATTGAGGTTTAGAGAGG |
| | | TGAAACGACCTACCCAAGTTTGCATTAGAGATAGTAATGTAC |
| | | AGATCTGAAATTCTAACCTAGGTCTTTCTCGTTTCAAAGTCT |
| | | CCATTTTTAACCACAAAAAAAGAAAGTGTCTGGGGTTGGAA |
| | | GATTTGCTAAGGCTTAAGCAGAGAAGCTAATTTGTAGCCTTA |
| | | AGAAGAGCTACAGCTTTCAGACAAGGGCAGGACTCTTGGGG |
| | | AGAGGTGGTGGTGGTGTTGCTGCTAGTGGTTTGTTTTTTGGG |
| | | GGAGAGACGTGGGATAGAGAATCTCTTCTCACCAGCATCAC |
| | | CACCACCTCATACAGAGCCTATGTTGAAATAGAAAAAAGTG |
| | | GCCTTTTTCTGGAGGATGCAGCCCCTACCAGGAAGTAACTGC |
| | | TTGGCTAGCAAAGTGCAAGCCGGAGTCCTGCCCCCATTTTCC |
| | | TTGTTGGCTTGGGCACCTGTGTGCAGTGAACAACCTGCAAAA |
| | | CTCTTCTTAGTGGTCTTCTTCCTGCCAACCCAGGAACAGCAG |
| | | CATTCAGCAGTTCTGTGCTTGATCCAAATGTGGCCAATTTTT |
| | | CCCCAGAGCACAGTGTATTGAAAATCATACCAAGGAAGAAA |
| | | ATGGAGGGGACTAGTAATTGTTCATGTTTCAGAAATTACAGA |
| | | ACTGAGAGCTTGACTGCTTCCTTCCCTTCCTGGGGATTTACA |
| | | TGGGGGAAGCAGAAGTGATTGGGGCTGAGGACATTCACATT |
| | | TCCTCACAACTGGAGGCAGTCATTAAGGATTTGGGCATGTCA |
| | | TAGGATTTGGGTGTTTTTCTCCCCCCTTAGTGGCATCTCCACA |
| | | GGCAGCGAAAGCTGTCTAAGCAAATAGGTGAGGCAGTGCGT |
| | | TAGTCCATTTTTTGTTGCTTAAAACAACATACCTGACACTGA |
| | | GTAATTTATAAAGAAAAAAAATATTTCTTACAGTTATGGAGG |
| | | CTGAGAAGTCCAAGGTTAGGGGCTGCATCTGGTGAGGGCCT |
| | | TCTTACTGGTGAGGACTCTCTGCAGAGTCCCAAGGCAGTGTG |
| | | GGGCATCACGAGGCAAGAGGTTTCGGTGTGATAGTCTCTCTT |
| | | CCTCTTCTCATAAAACCACTAGTCTTACTCCCACGACAACCC |
| | | ATTAACTTATTAACCTGTTTATCCACTAATCCATGAACGGAT |
| | | TAACCCATTCATCAAGGAAAAGCCCCAGTCACCTCTTAAAG |
| | | GCCCCACCTCTCAATACTGCCACATAGGGGATTACATTTATA |
| | | CATGAGTTTTGGAGAAGACAAACATTCACGTCATAACAGGC |
| | | AGCTTCACCCAAGTCTGCTCCTCAAGTCTGTGGGTTTTCTCTC |
| | | TGCTTCTGGAAGGTGAAGCCCAGGCCTGGGCAAGTATTCTTT |
| | | TGCAGCATCCTGAAATATGACCTTAGCTTCTAAATGTGTAAA |
| | | ATGGAGATAATAATCACACCCACCATCACGGGATGGTTGTA |
| | | AGACTGAATAAGTTACAATGTGTAAAGCATTTAGAAAAGGA |
| | | ATTTGCACAGAGTAAGCATTCAGTAGATGTTAGGTACTACCC |
| | | CACAACCACCGTTATCATTATTATCACCATCATTATTATCATC |
| | | TTCATTTTCATCACCTTCATTATGGCCATCATTCCATTCCTCA |
| | | GCTCAGAAGCCTTCTATGGCTCCCCATTGCTTATAAAATCAA |
| | | GTCCTAACTTTCCCCAGCATGAAACTTCTACCACCGTCACCT |
| | | ATCTCTCAGGGTTTCCTAAACATACTCTGAACAAGTTTCCTC |
| | | ACTCTGCCACTGTGACCCAGAAAGCTATCTGTTCTCCTTCTC |
| | | CCAGACTCTGCCTCATCTTTCAAGGCCTGGTTCCAGGATCCC |
| | | TTCCTCCAGGAAGCCTTCCCTGGTTGCCCTATTCCACCATAC |
| | | ACCTTTTTTCTCGGACTTCATGTCATGGTGCCTATATTCCAAG |
| | | GGCTCTGAGCCATGTACCCCTTTATATAGTTACCACTATTTA |
| | | CTGAGTGCCTACTGTATACCAGCTACTGTGTTGGATGCTCTA |
| | | GATGTAGAACCTCTAATTATCACCATCGATTCCTGGGTAGTA |
| | | GGCATTATTTATTCATCTTACACAGATGAGAAAATGGAGGCC |
| | | CACAGTGGTTAAATAACTAGCCCAAGATTGCACAGCTAGTA |
| | | GGGCTAGTGGAAAGTAGAGGTGGAATTTGAACTCAAATCCT |
| | | GCAGTAACTCTACCATTCTGCCTTGCTCTTCTTTGTAGCAGTA |
| | | GATAAGTTTTCATGGATACATACCTCATCCTTTTGATTAGATT |
| | | AAGGGCCCCTGGAGTGTCAGTGTTCATTCATTTGTTTGATCA |
| | | TTCATTCATTCAACAAACATTTCTTGAGTCCCCACTGTGTGCC |
| | | AGGCCCAGAGGTTCCCCAGCCCAAGGCCTGGCACACAGTGG |
| | | GCCTTCAGTTAGACCTTGTTGATTGACTGCGCTTTTCCTTGTC |
| | | TGGGCAGCGGAACTGGACCAGTATGTCTTCCAGGACTCCCA |
| | | GCTGGAGGGCCTGAGCAAGGACATTTTCAGTAAGTTTGTGGT |
| | | GGGTGGGGAGGTCTTGGCTCAGCCTGCATTTCCTGCCTTGTT |
| | | CCCTGGGGGGTGCCCTAATACCTGACGACCATTCATTGATGG |
| | | GCAGTCAGACCCCTCTCCCCAAGGTGGGGACAATAGAGACT |
| | | CACCTTGGGCTTTCATTGATTGTGTGAGTTGGTCTCTGGTTTT |
| | | TCTCAAAGTAGAGCACATAGGACCAGATGAAGTGATCGGTG |
| | | AGAGTATGGAGATGCCAGCAGAAGTTGGGCAGAAAAGTCAG |
| | | AAAAGACGTGAGTGAGCCCCTCCCTGATCCAACCTAGCCTTG |
| | | CTTGAGACCTGGCCTTTCCTTGACTCCAAAGCCTGCTGTGGG |
| | | TCCAACTTGCTTCCCTCGCTAAGTCCTGTCTGGTTGGGAGGC |
| | | CCTTTAAAAGCCAACAGGAGCCTTAAAATGTACATCTGATTA |
| | | TTTCATGGCCCTGATAACCCTCCAATGGCTACAAAATACATG |
| | | CCACAAGGCCTGTATGGCCCTTCCTCCCTCTCCAAACCCACT |
| | | ATGAGACACCACACTTCAGCCACCACCAGCTTCTCCACTCCT |
| | | ATAACCCATGTGCCCTTTCAAGCCTCGGGGCCTTTGCAGTTG |
| | | CTATAGTCTCTATCTGGAATGCCCTTCCCCCAGTTCTTCCCAT |
| | | GGCTGACTCCTTTGAATCTTTCTGGTGTTGGCTAAACTGTCA |
| | | CCTCTTCCTGGAACCCTTCTCTGACCATCCTTCCATGTAGATT |
| | | AGCTCAGTTATTCTCACCTTGTGTGTCTTTTTCCTTGCAGTTT |
| | | AGCACTCATTACCATCTGGACATATTTTACGCCTTGCTCTCCC |
| | | ACTGTGAGGACAGGGACCTTGTCTTTCTTGCTCGTGACTGTT |
| | | TCCCCAGCATCTAGTGCAGTGCCTGGTATGCAGTAGCACCTC |
| | | AGTAGATATCTGTTGAATGAAAACATCTGTAAAATGGGTGT |
| | | AACAGTTAACTGAGTACTTATTATGGGTCTGACCATGTGTAA |
| | | GTCCTGTATCTATTTATTCAGTTCTTAAACAGGTGAATCGCA |
| | | CACAGGGTATGAGATTTTAAAAGTGCAAAGAATATTCAGTG |
| | | AAGGCTGGGCGCAGCGGCTCACACCTGTAATCCCAGCAGTT |
| | | TGGGAGGCCAAGGGGGACGGATCACTTGAGGTCAGGAGTTT |
| | | GATACCTGCCTGGCCAACATGGTGAAACCGCGTCTCTACCAA |
| | | AAAATACAAAAATTAGCCGGGTGTGGTGGTGCACGCCTGTA |
| | | ATCCCAGCTACTCGGGAGGCTGAGGCAGGAGAATCGCTTGA |
| | | ACCCAGGAGGTGGAGGTTGCAGTGAGCTAAGATCACGCCAC |
| | | TGCACTCCAGCCTGGGTGGCAGAGTAAGACTCTGTCTCAAAC |
| | | AACAACAACCAAAAGAATATTCAGTGAAAAAAAAATCTCCC |
| | | TCCCACTCCTGTTCTGTAGTCCCTCAGTTTTGCTCCTATCTTA |
| | | GTCAACCTATGTGACCAGCTTCCTTTTTGCCTTGTCAGAAAT |
| | | AAAGCGAGCTCCTGTATTTGCTTTAATTTTCCATGCAAATGA |
| | | CCACATTTTATTTACACTATTCTGCATTTTGCTTTCTTCATTTA |
| | | CTTCATTACTCCCTGTCTGGGAGGTCACGTTCCATATTATTGA |
| | | GGGAGAGGACACGGTCCTATCTTCAGCAGCTTCTTTATTTTC |
| | | TTTCTTTTCTTGTTTTTCTTCTTTATGTTTTCATTTGATAAATA |
| | | TTTCCCCCCAAGATTTTATTGTGAAAGTTTTCGGCCAGGCGT |
| | | GGTGGCTCACATCTGTAATCCCAGCACTTTGGGAGGCCAAG |
| | | GCTGGCAGATCACCTGAGGTCAAGAGTTTGAGACCAGCCTG |
| | | GCCAACATGGTGAAACCCCATCTCTACTAAAAATACAAAAA |
| | | TTAGCCAGGCGTGGTGGCGAACACCTGTAATCCCAGCTACC |
| | | AGGGAGGCTAGGGTGGGAGAATCGCTTGAACCTAAGAAGCA |
| | | GAGGTTGCAGTGAGCCAAGATTGCGCCAGTGCACTCCAGCC |
| | | TGGGCAACAGAGCTAGACTCTGTTTCAAAAAAAAAAAAAAA |
| | | TGTTTTCAAGATACAATAAAAGTGAAAGAATGGTACTATGA |
| | | AGGTCCATATACCCACCATGTACCATTTTGATTCTACAATTA |
| | | ATGTTGGGTTTTATTCATCTTATCACATCTCTATTCATCTGTC |
| | | CATCCCTCTATCCATCCACACACCCATTCATTATATTTTTGGT |
| | | ACATTTCAATGTACATTTCAAATGTAAATTGCAGATTTACAC |
| | | TCCCCTCGTGTACTTCAGCATCCTTTCTTTTAAGGCCATGCAG |
| | | TGTTTCGTTGGGTAGATGAACCATAATTTGTTTAAGCAGTCC |
| | | CTTGCTAAGGGACACGTGGGTTTTTAATCTGTTGCTGTGAGT |
| | | GCTTGATACTCACTCACTGATTTCTGGCATACTCCTGCAATG |
| | | TAGGAACCACCACCCCCATTTCATAGATGTGAGATCAAAGG |
| | | TTCAGAGAAGCTGCATTGCAAAGGCACACAGCTAGGAAGCG |
| | | GTTCAAAAGCAGGTTCCTTTGTTTTGTTTTGTTTTGTTTTTTG |
| | | GCTCAAACCCATGTTGTGTCCCCATTTGTGACTCGGCCTCTTC |
| | | TGCTGCTACACTGCCTGGTATGGTTTGAGATGGGGTTTGGGT |
| | | TCAAGCCAAGGAGCTGGGGTTGGAATGTTAGAGCGAGGGGA |
| | | GGAAAATGGACCCCCAAGACCACTACCCAGCCTTGAAGTTA |
| | | AGGCCGTATAGCCTGCTAGAGTCCTGAGCCCCTTCTGGCTTG |
| | | GGACATCCTCTCCCTGGGGCAGCTGATCACATGTTTTCTCTG |
| | | CAGCCTTCCCAGAGGAGCTTCCGGCAGACCTGAAGCACTGG |
| | | AAGCCAGGTGTGCAGGGCAGGTGGGCTGGGGTTGGGAAGGG |
| | | TGGATGCCTTGGGGAGGGGATGGAAGAGATTGAACTCCTGG |
| | | CCCAAGTCTGATGGGGATGGTGCATGGTGCAGCCCCTGCCCT |
| | | TCTTTGGGTAGAGGCTGAGAGCTTGGGGTCCCTTAGAGTCCT |
| | | CTAAGGGGCTCACGACTGTTTGTGGAAGGTGGTAGGGGCTT |
| | | GGAGCTAACAGATTGTTCATAGGTTCTATTCTGCCCCAGCTC |
| | | TCCGTGTGGAGGCCCTAGGGTCCTGCTCAGCATAGCTCTCAG |
| | | AGCCAAGTCACAAGGAGAGGACTGGGGGACTGCCTGGCACA |
| | | GAGCAGTTGCTGATCAACACAGCTGCAGCCAGGGCTGAGAA |
| | | GATGACAAGCATTTCCTCTGTCAGGAGAGACATCCATGCCAC |
| | | TCCAGGGCCCTCCCCATCCCAGGAAGGCCCCTCCAAGCACCC |
| | | AGTCTCTAACACAGCCCACTTCCTCACAGCTGAGCCCCCCAC |
| | | TGTGGTGACTGGCAGTCTCCTAGTGGGACCAGTGAGCGACT |
| | | GCTCCACCCTGCCCTGCCTGCCACTGCCTGCGCTGTTCAACC |
| | | AGGAGCCAGCCTCCGGCCAGATGCGCCTGGAGAAAACCGAC |
| | | CAGATTCCCAGTATGTTAGGGGGCTTGGAGAGAGTGGGCTTT |
| | | CTCCCTCTTGGGAGGTGGATAAGGAGTTGACACTAAGGCAG |
| | | GGACTGTCAGGAACTGGACCTCACCTCTCCCCAACCCTATCA |
| | | GTGTCACTCACCTCTGCCCCAGCCAGTTTTATCCTTGGGGCC |
| | | CCCGTCAGCTCAATCCCTGGCAGAGAACATGCCTCAGAAGG |
| | | AAACACATCCGTGCGAGCATCAGGAGGGAATCTCCACCAAT |
| | | TAGAGCATTCACCTGCCAGCCTTATTCGTTCATCAGCTCTTC |
| | | ATGGAGCCCCTTCTCTGTGCCAACTGCTCTGCTAGGTGCTAA |
| | | GAATTCCATCAGGGCAGGACAGAAACAGCTACTGCTCTTAG |
| | | GGAAATTAGGGCCCTTTAGGGGGGTCAGACATTAATCAAAT |
| | | AAGCAAAAGCAGAATCGCAAACACAGGTGCTATGCAAGATC |
| | | CCACCTCACTGCCTTTGTCTCTTGCAGTGCCTTTCTCCAGTTC |
| | | CTCGTTGAGCTGCCTGAATCTCCCTGAGGGACCCATCCAGTT |
| | | TGTCCCCACCATCTCCACTCTGCCCCATGGGCTCTGGCAAAT |
| | | CTCTGAGGCTGGAACAGGGGTCTCCAGTATATTCATCTACCA |
| | | TGGTGAGTGCGGGGCCTGGCTCCCCGACCACCTCTCCCTCCT |
| | | ACCTGACTGCTCCCTGACCTCATCCTCCCCATACTCCATGCA |
| | | CTTGGCAGTGGTGCCCTAGCACCTTCTCATGATCCTCCCTCC |
| | | CCAGCACAACTTTCTCTGTCCCTATTTCCCAAAAATCAAAGC |
| | | AGAAGAGCAGAGATTCCTGAGGTGGTCTGGGTGCATGACCA |
| | | AGAGTGGTTGTGTGGCCTTCCATATGTTGCTTTCCCCTTCTGA |
| | | CCCTTAGTTTTTCCTTATTTTTTTTAAATGTTTTTGTTTTTAAA |
| | | TTGCAACATCAATACATGCTCACTAATAAGCTAATGACAAGT |
| | | GATGTTTACAAGAATTCACTGTCTCTCTCACTCTTTTAGTATA |
| | | CTGATGTGAATAGTTTGGTGTGCATCCTTGCATAGCTTTCTCT |
| | | GATTTGTCTTGTTTCCTAATCAGTAAAATGGTCCCTACTGAC |
| | | CCACCACACTGATGGGTTGTGACTTGTCTCAGGGAAGGTGTG |
| | | TTTGGGCTGTCAGGGATGAGACCAGGAGGATGCTTCCACTC |
| | | AAGTCCAGAGCATCCATTCCTGGTTGCAAGACAGTTTCAGTA |
| | | TAACGCACTGGTATCTCAGAAGCAGAGAAGCAGTTCCTGAT |
| | | ATCTAGGAAAATGAGCACTGACCACACAGCAGGAAGAGTGC |
| | | AATAGTAGTACCTGCCCTAAGTTCCTTATGAGCTAATTGCAC |
| | | CCATTTCACAGATTGGAAAAGCAAGGTTCCAGGAGCTAGAT |
| | | GTAACTCCAGAGCTCTTTCTTCAGCTCTGTCCTGAAATATCTT |
| | | CCTTTGGAAGTTCTACTCTCTTCTCTCCAGACTTCCTGAGCTC |
| | | CACAGCCCAGTTTGGAGTAGGGGTGACCCAAGTGCATTTCTG |
| | | GAATCAATACCTGGTTATTCTCACACCACTCTCCACCCCCAA |
| | | TGTAGGTGAGGTGCCCCAGGCCAGCCAAGTACCCCCTCCCA |
| | | GTGGATTCACTGTCCACGGCCTCCCAACATCTCCAGACCGGC |
| | | CAGGCTCCACCAGCCCCTTCGCTCCATCAGCCACTGACCTGC |
| | | CCAGCATGCCTGAACCTGCCCTGACCTCCCGAGCAAACATG |
| | | ACAGGTAAGGACCCTTAGGGCCTGTGAGAGGTACTAGAAGC |
| | | AGGATCGAGGCCCTGGGGAAGGAATTGTCTCAAATAAGATC |
| | | CACAAGCAAAGCTGCCTGTAGGGACAACAGGTCATGTTTAG |
| | | GGGTCAGTCGGGACAGGGAGGGTCTCCAGGGAGTGGAGGCA |
| | | TGGTTGTGGGGGAATGGACAGCTAGAGAATCCACATCCCAT |
| | | CCAAATACAGCCATCAGCCTTCAGCTGCTTGTTACCCAGCAG |
| | | AAAAGCCAGACAATGTGGTCAGATCTTTTGAAGTTGAAAAC |
| | | CCTGATTTTTACATAGATATTGTGATTTTTTAATTTTTATTTT |
| | | ATTATTTATTTATTGTTGAGATGGAGTCTCACTCTGTTGCCCA |
| | | GGCTAGAGTGCAGTGGTGCGATCTCAGATCACTGCAACCTCC |
| | | GCCCTACAGGTTCAAGTGATTCTCCTGCCTCAGCCTCCGGAG |
| | | CAGCTGGAATTACAGGTGTGCACCGTCACACCCAGCCAATTT |
| | | TTGTAATTTCAGTAGAGACAAGTTTAACCATGTTGGCCAGGC |
| | | TGGTCTCGAACTCCTGATCTCAAGTGATCCACCTGCCTCGAC |
| | | CTCCCAAAGTGCTGGGATTACAGGTGTGAGCCACTGCACCC |
| | | AGCCTGATATTGCGATTTTCTTTTTTAAACATGAGTGAAATTT |
| | | TTAAAAGTACAGTTGGCCAAAGAAAGACATATTTGAGTTAG |
| | | ATACAGCCTCAGGCCGCTATCTTATAACCTCCATCTTGGGAA |
| | | ACTCAGCCCATGAAGGCTGTAAGGACTAAGTGGCCCAGAGG |
| | | GAGGGGGGTCCCCAGGGGTAACCCTCACCCTAAATCTGGCA |
| | | CCTGCTTCTCCATCTCCAGAGCACAAGACGTCCCCCACCCAA |
| | | TGCCCGGCAGCTGGAGAGGTCTCCAACAAGCTTCCAAAATG |
| | | GCCTGGTGAGTGATGCGGGATCTCTCTGCCCTGGGTGGTGGA |
| | | GATGGAAGCCCATATCTGGCTTCACATTGCTCATTCACTTGA |
| | | CACTTATTCAACCCCTTCTTTGTTGGCTCACACACTCATTTAT |
| | | TTATTCATTCATTCATTCATTCACTTATTTGATTATGCCATCA |
| | | TTTCACCATTCACTCCACCACTCCCTCAAATAATATTTATTGC |
| | | CCTTCAATAAGCCTGCACTGGGTGTTAACACTTCATTGTCAG |
| | | GCTGAGAGAATGAATGAGGGGCAAGCGAATGATGAAGTGA |
| | | GTGAGTGGATGAAGTGAGAAGTTGATGAGGATGGGAGCAAT |
| | | GGAAAGTGGGAATAAACCAGTGAATAAGAGAATTACTAAAT |
| | | TACTAAATGCTTACTCTCACATACTGGTCTAAGCATGTTAGG |
| | | TATGCTAAATCATTAACATCTCACACAATCCCAATATTATCC |
| | | CCATTTTCCAGATGAGGCTACTGAGGCATGGTGGGATGTATA |
| | | GCATGCTCAGAATTGCTCCAGCAAGTGGTAGAACCGGGATT |
| | | TGAACCCAGACACGTGGGCTCCACTGTCTTAACTCTTAACAC |
| | | TCCCTCCCATGACCTCCCCATGGTGAGTGAGTAGTGATCAAG |
| | | CGAAGAAAAAGCTTAGTGAGTAAATGAAGGAATGAGAAAA |
| | | GATGGCCATGCATGGTGCCTTATGCCTGTAATCCCAGCACTT |
| | | TGGGAGGCCAAGGCAGGTGGATCACCTGAGGTCAGGAGTTC |
| | | GAGACCAGCCTGGCCAACATAGTGAAACCCCGTCTCCATTA |
| | | AAAAATACAAAAATTAGCCAGACGTGGTAGTGGGTGCCTGT |
| | | AATCCCAGCTACTTGGCAGGCCGAGGCAGGAGAATCGCCTA |
| | | AACCCAGGAGGTGGAGGTTGCCGTGAGCTGAGATTGCGCCA |
| | | GTGCACTCCAGCCTGGGGCACAGGGCAGGATCCATCTCAAA |
| | | AAAAAAAAAGAGAGAAAAGGTCTAAAAGGAGTGGACACTT |
| | | AAACAAGAGAGACGATGAGTGAAGGAATGGGTGTGGAAAT |
| | | GAGTGAACTAATGAATGAGTGGTGGGTGACTGAATGAAGCA |
| | | AATGATGAAGACTGTATGGGGGCCAGATGTGGTGGCTCACA |
| | | CCTGTAATCCCAGCACTTTGGGAGGCCAAGGCGGAACGATT |
| | | GCTTGAGCCCAGGAGCTCAAGACCAGCCTGGGCAAAATAGT |
| | | GAGAACTTATCTCCACAAAAAAACAAAAAAGAAAAATTAGC |
| | | CAGGCATGGCAGTGGGCGCCTGTAGTCCCAGCTACTCAGGA |
| | | GGCTGAGGTGGGAGGATTGCTTGAGCCTGTGAGGTTGAAGC |
| | | TGTCGTGGACCATGATTGTGCCATTGCACGCCAGCCTGGGCG |
| | | ACATAGTGAGACCCCATCTCTACAAAAAAATCAAAAAATTA |
| | | TCTGGGCATGGTGTCACATGTCTGTGGTCCCAGCTACTTGGG |
| | | AGGTGGAGTCAGGAGGATCGCATGAGCCCAGGAGGTTGAGG |
| | | TTGCAGCGAGCTGTGATCACACCACTGCATTCCAGCCTGGGC |
| | | AAAAAAGCCAGACCCTGTCTCAAAACAAAACAAAACAAACA |
| | | AACAAAAAAACTGTGTGGGGAACAGATGTAAATGATGGTGG |
| | | CAGTGCTGGCCTTGTGGTGGCTGGCCCTGGCCCTGCCTCTCA |
| | | CATACCCCCACCCTGACACGCCCCTGGCCTTTGCAGAGCCGG |
| | | TGGAGCAGTTCTACCGCTCACTGCAGGACACGTATGGTGCCG |
| | | AGCCCGCAGGCCCGGATGGCATCCTAGTGGAGGTGGATCTG |
| | | GTGCAGGCCAGGCTGGAGAGGAGCAGCAGCAAGAGCCTGG |
| | | AGCGGGAACTGGCCACCCCGGACTGGGCAGAACGGCAGCTG |
| | | GCCCAAGGAGGCCTGGCTGAGGTGCTGTTGGCTGCCAAGGA |
| | | GCACCGGCGGCCGCGTGAGACACGAGTGATTGCTGTGCTGG |
| | | GCAAAGCTGGTCAGGGCAAGAGCTATTGGGCTGGGGCAGTG |
| | | AGCCGGGCCTGGGCTTGTGGCCGGCTTCCCCAGTACGACTTT |
| | | GTCTTCTCTGTCCCCTGCCATTGCTTGAACCGTCCGGGGGAT |
| | | GCCTATGGCCTGCAGGATCTGCTCTTCTCCCTGGGCCCACAG |
| | | CCACTCGTGGCGGCCGATGAGGTTTTCAGCCACATCTTGAAG |
| | | AGACCTGACCGCGTTCTGCTCATCCTAGACGGCTTCGAGGAG |
| | | CTGGAAGCGCAAGATGGCTTCCTGCACAGCACGTGCGGACC |
| | | GGCACCGGCGGAGCCCTGCTCCCTCCGGGGGCTGCTGGCCG |
| | | GCCTTTTCCAGAAGAAGCTGCTCCGAGGTTGCACCCTCCTCC |
| | | TCACAGCCCGGCCCCGGGGCCGCCTGGTCCAGAGCCTGAGC |
| | | AAGGCCGACGCCCTATTTGAGCTGTCCGGCTTCTCCATGGAG |
| | | CAGGCCCAGGCATACGTGATGCGCTACTTTGAGAGCTCAGG |
| | | GATGACAGAGCACCAAGACAGAGCCCTGACGCTCCTCCGGG |
| | | ACCGGCCACTTCTTCTCAGTCACAGCCACAGCCCTACTTTGT |
| | | GCCGGGCAGTGTGCCAGCTCTCAGAGGCCCTGCTGGAGCTT |
| | | GGGGAGGACGCCAAGCTGCCCTCCACGCTCACGGGACTCTA |
| | | TGTCGGCCTGCTGGGCCGTGCAGCCCTCGACAGCCCCCCCGG |
| | | GGCCCTGGCAGAGCTGGCCAAGCTGGCCTGGGAGCTGGGCC |
| | | GCAGACATCAAAGTACCCTACAGGAGGACCAGTTCCCATCC |
| | | GCAGACGTGAGGACCTGGGCGATGGCCAAAGGCTTAGTCCA |
| | | ACACCCACCGCGGGCCGCAGAGTCCGAGCTGGCCTTCCCCA |
| | | GCTTCCTCCTGCAATGCTTCCTGGGGGCCCTGTGGCTGGCTC |
| | | TGAGTGGCGAAATCAAGGACAAGGAGCTCCCGCAGTACCTA |
| | | GCATTGACCCCAAGGAAGAAGAGGCCCTATGACAACTGGCT |
| | | GGAGGGCGTGCCACGCTTTCTGGCTGGGCTGATCTTCCAGCC |
| | | TCCCGCCCGCTGCCTGGGAGCCCTACTCGGGCCATCGGCGGC |
| | | TGCCTCGGTGGACAGGAAGCAGAAGGTGCTTGCGAGGTACC |
| | | TGAAGCGGCTGCAGCCGGGGACACTGCGGGCGCGGCAGCTG |
| | | CTGGAGCTGCTGCACTGCGCCCACGAGGCCGAGGAGGCTGG |
| | | AATTTGGCAGCACGTGGTACAGGAGCTCCCCGGCCGCCTCTC |
| | | TTTTCTGGGCACCCGCCTCACGCCTCCTGATGCACATGTACT |
| | | GGGCAAGGCCTTGGAGGCGGCGGGCCAAGACTTCTCCCTGG |
| | | ACCTCCGCAGCACTGGCATTTGCCCCTCTGGATTGGGGAGCC |
| | | TCGTGGGACTCAGCTGTGTCACCCGTTTCAGGTGGGGTGAGG |
| | | GGCTTGGGGAAGAGACATCCTTGTGTTGGGCATTAACTGCG |
| | | GTCTTGGTGCCAAGCCCAGTGCTCTGTGGGGTCTCTTTTAGT |
| | | ATGCAGAGCAGCCGGGTGGGGCAGAATGGATTGTCTCCATT |
| | | TTTAAGATGAGGATGTTGAGGCTCAGAGAGGGGCAGCCACT |
| | | TGCCACACAGCAAGTGAGAGGCAATGGCATTCTCCCAGTCA |
| | | ATATTTGAAGGCCCGCCATGTGCCAGTCACTGGGGTATGTCT |
| | | AGAATCTGAGACTGACCTGGGCTCAAATTTGTTTTATTCTTT |
| | | CCACCCCCTGAGCACGCCACCGTTTTCTTATGCTAAGAGTAA |
| | | AGCCATGGCCTCCCCTTGGACTCTCTGCCTCCATTCTCTCCTC |
| | | TTCCACTCCATTTTGTATTCAGCAACCAGACCAATCTTCTCA |
| | | GAACTTGAATCTGATTGTATCCCATCCCTGCTTACAATCCTTC |
| | | AGGGACACTCCACCACTGTCAGGATGAAGGCTAAATTCCTT |
| | | AATTTGGTTTCATTAAGTCGGTCTGCAATCTGCTTGAGCATTT |
| | | CAGCTTAATCGCCAGAGGATTGCTTCCATATTTCCCCCTAAA |
| | | CATACTTTACCCAAGCTGTAAGATCCTACATAATTGTGCCAA |
| | | TAATTTAGCAGTGAGCTTCCTGGTAGCCGAAGCAAAAAGGG |
| | | AAAGAAAACCACTGTGTGAGTTGTGAGAAAGTAGGAATCAA |
| | | TAAAGGCTGGAGTGGTCATGGAAGGCTTTCTGGGAAAGGTA |
| | | GAGGTTGAGCTAAGGAAAGAAAGTATTTTAATAGGTAGGAG |
| | | GACCCTTCATGGAGCTGCCCTTCCATTAAGGTCTAGCCTGGT |
| | | CACCGTGCCTGGGTCTGAGGCCCTCCCTCCACAGGGCTGCCT |
| | | TGAGCGACACAGTGGCGCTGTGGGAGTCCCTGCGGCAGCAT |
| | | GGGGAGACCAAGCTACTTCAGGCAGCAGAGGAGAAGTTCAC |
| | | CATCGAGCCTTTCAAAGCCAAGTCCCTGAAGGATGTGGAAG |
| | | ACCTGGGAAAGCTTGTGCAGACTCAGAGGTGAGAGGAGAGG |
| | | CGGATGGGAGGTGGTTCACGCCATGCAGGTTGAGGACATGT |
| | | AGGACCCATTCTCAAGTTTGTCATGGGCATTTCCAGTGCCCC |
| | | CTGTCCTCTGGGACACCCCATGCCCTCTTTCTGGGCAATGGC |
| | | TAACCAGAGTCTCTCACTGCACTCGCTGCCCAGGCGGAGCCT |
| | | CTAGAATTGAAAACTTTGCTGCATTCAAAACCTGCCATCATG |
| | | TGCCCAAGTCTTAGCTAAAGCCATGGAGAACCTCTCCCCTTC |
| | | TAGGAGTTAACAAGGATGCCTTGTGCACCTAGTAACAGAAG |
| | | TGACACCTACTGCCACCCTTTGAGGCATTTAGGACAACTGTT |
| | | GTCCCAAATTTATAGATGGCAGAGCTAAGGTCCAGAAGGCA |
| | | GACAAAACTTGATTACAGAACAAGTTAACTGTCTTTCATTCA |
| | | TTCCTTCATCACACATTGTTTGAGCGCCTCTTAAGTGCTGTCT |
| | | GGGATAGGTACTGGTAAGTGCCTAATCTGAGGCTCATTTCCA |
| | | TGAAAGATACAGTCCTGAGATCTTTTATTTTTATTTTTATTAT |
| | | TTCTTAAAGACAGGGTCTCGCTGTGTCTACCAGGCTAGAATA |
| | | CAGTAGTGCGATCATAGCTTACTGTAGCTTCGGAACTCCTGA |
| | | GCTCAAGCGATCCTCCTGCCTCAGCCTCCCAAGTAGCTGGGA |
| | | TTATAGGTGCACGTCATCACACCGGATGATTTTTTAAGTTTTT |
| | | GTAGACACAGGGATCTTGCTATGTTGCCCAGGCTGGTCTTGA |
| | | ACTCCTGGCCTCAAGCAATCCTCCTGCCTCAGCCTCCCAAAG |
| | | TGCTGGGACTACAGTCACGAGGCACCGCACCCAGACAGATC |
| | | TTTTTTTTTAAGAGGGGGACAACAGTTGCCCCAAGGAAAGC |
| | | AATCCCCCTGGGGAATTTGGGGCACTGGGGCAGCCATCATG |
| | | GGACCCATGGGTAAGGGCTCAGTGACAGCAGTGCCTGCTCC |
| | | CCCTAACATTGCCTGTTCTCTCCAGGACGAGAAGTTCCTCGG |
| | | AAGACACAGCTGGGGAGCTCCCTGCTGTTCGGGACCTAAAG |
| | | AAACTGGAGTTTGCGTAAGCAAAGGGGTGGATTGTCTTGTG |
| | | GGTCTGCGCAAGGTTTCCCCTGCAGCATTAGCTGGGCTTGTA |
| | | CCACCTGAATGGAGATAGATCTCCAGAATCATTCTTACCCTC |
| | | TTGCCCACCACTTTGGAAATAGCTTCCAGCAGCTGGAAAGTG |
| | | ACCAGTGTGGGCAAATGCTGGAGGTTCTCAGATTTGAAGCT |
| | | AATGGACCCGTTTGCTGGTTAACATGGTCATTCCCATCACTC |
| | | ATTCAACAGACGTATTGAGCACTTGAATGACTGAGGCTGCTG |
| | | TCCTCATGAGCTGACAGTCTAATCAACTCCCCTCTGGAGGAG |
| | | GTCAGGGGGACTTCCTGGAGGAGGTGATATCTGCACTGAAG |
| | | CTTCAGTGAGGATAAGGAATGAGCTGAGTGGGGAGAGGGGA |
| | | GTAAGAAAGGCTTTCCAGGAATTGTAATTTGGCCAGGGATC |
| | | AGAAAAGGCCCAATGTGTCCCCAACCTTCCATAGGAGGATG |
| | | CCACTATGACTATGGAGTCCTTTTCCCTCCTGTGGCCAGGAA |
| | | CTGCACAACGTCCCTTGTGCCTGAGCTTGTTTACTTCCACAG |
| | | CCAAGGGATGGCCTCCCCAGGGAGAATCTTTCCCCCATTTTG |
| | | AAATTCCCAGGAGCTTTTGATGTGCAGCTAGGATGAAAAAG |
| | | CAACCCTACTTGCCTGGGACTATCCTGGTTTGAAAACTGAAA |
| | | GTCTTGCATCCCAAGAAGCCCCTTAGTCCCAGGCAAACTGGG |
| | | ACAGTTGGTTGCTCTATGTGTGGCCAGGGCTGGGGGCCACAC |
| | | CCTAGGCAGTGTTGCTGGGGTGACAGAAATGGAGACCTAGG |
| | | CAAGGCTCTAGGGAGTCCAGTTGTGTCTGGGTCACTGAGGA |
| | | GGGGCAGCCCACCATACATGGGCCCCAAGAAAGCACAATAA |
| | | TTATTATATTTAGATCTGTCAGAATATAGCTTTTCTTTCTTTC |
| | | TTTCCTTTCTTCTCTCTTTCTTTTTCTTTCTTTCTTCTTTCTTTC |
| | | TTTCTCTCTTTCTTTCCTCTTTCTTTCCTTTCCCTTCCTTTTCTT |
| | | TCCTTCCCTCCCTCCCTCCTTCCTTCCTTCCCTCCCTCCCTTCT |
| | | TCCCCCTTTTCTTCTTTTTCTTTCTCTCTGTTTCTTTCTTTCTTT |
| | | CTCTCTCTCTCTCCCTTCCTTCCTCCCTCCCTATCTCTCTCTTT |
| | | CTTTCTTTCTTTCCTTCTTTTCTTTCTTTCCTTTCTCTCCTTTCT |
| | | CTTTCTTTCTTTCCATTCTCTTTCTTTCTCTTTCCTTCCCTCCCT |
| | | TCCTTCTCTTCCTTCCTTCTTTTTCCTTTCCTTTTCTTTCCTTTT |
| | | CTTTTCTTTCTTGAGGCAGAGTCTTACTCTGTCCCTCAGGCTG |
| | | GAGTTCAGTGGCACAATCACAGCTCACCACAGCCTCAACCTC |
| | | CCTAGGCTCAAGTGATCCTCCCACCTCAGCCTCCCTAGTAGC |
| | | TGGGACTACAGGCATGCCCCACCTGACTAATTTTTGTATTTT |
| | | TTGTAGAGACAGGATTTCACCATGTTGCCCAGTCTGGTCTCG |
| | | AGCTCCTGGGCTCAAGCAATCCTCCCACCTCAGCCTCCCAAA |
| | | GTGCTAGGATTACAGGTGCGAGCTCCCGCACCTGGCCAGAA |
| | | CAGGCTTTTAAATTTGCAGCTCACCTTCCATCTCAATTATCTC |
| | | AGGCCACCCTCCTCTACAGGCATTGTCATCATCTCCCCATTG |
| | | TACAAATGGCAATGCTGAGAACAAGAGACTTTAAATTGTTT |
| | | ATCCAAAGTCATAAAGGTCAGGTCAGCCTGGCCCTAAGCTC |
| | | CTTCCACAGAAAAAGCTAAATGAATGTTTGTTCCTTGGTAGG |
| | | GGTCTAGATCATCTAATGATTAGGAAGGAGTTGTTATGCCTG |
| | | TGGTATATTCGGTGCTGTTTTTTTTTGGAGATGGGGTCTTGCT |
| | | CTGTCACCCAGGCTAGAATGCAGTGGTATGATCTTGGCTCAC |
| | | TGCAATCTCCACCTCTCGGTTCAAGCAATTTTCCTGCCTCAG |
| | | CCTCCCGAGTAGCTGGGATTACAGGTGTACACCACTACACCT |
| | | GGCTAATTTTTGTATTTTTAGTAGAGACGGGCTTTAACCATG |
| | | TTGGCCAGGCTGGTCTTGAACTCCTGGCCTCAGATGTTCCTC |
| | | CCACCTCAGCCTCCCAAAGTGCTGGGATTACAGGTGTAAGCC |
| | | ACCATGCCCAGCTTATAATTCAGTGCATTTTATATATAATCT |
| | | CATTAATCTCAAGAGCTACTGTGGAAGACAGACAGTCATCA |
| | | TCTTCCCCATTTTACAGATGAGGAGACGGAGGCCTGGTGAG |
| | | GTTAAGTGACTTGCCCGTGGTCCCCCAGCTGGTCACCCACAA |
| | | GCTATCAGCTCTCCCTGATCACTGCTCCCCTGTGCCCCAGAA |
| | | AAAAGCCAGTGTCTCTTGTTTTGAACAGAGACAAGGTGCTA |
| | | AATTTGTCCAGAGGTAAAAATGATGCTGAAAAGGGGAAATG |
| | | TCTGAAAACAATAACTGAGGGCCCTTTCTGGCCTTAAATTAT |
| | | TTTCAGCTGAAAAAGCCCCTAGTTCAAGATCGGAACTGCGCT |
| | | CTGGGGAACAGCGGCCATGGGGTGGAACAGCCTAGAGCCGG |
| | | GTGATCAGCCACCTGGAGACCGGAAGCCCCCGAGGAGGTTC |
| | | TGAGGAAGGAAGGAAGGAAGGAAAAGACTATTTCTGGCATT |
| | | TTCTCAGGCTTCTACTGTGCCCAGGCACCCTGTAGACCTCAT |
| | | CTGTCTGTCAAATCAGGGTTCTTAGCCACTCACAACCCTACA |
| | | GGGACTTCGTTTGTACACAGAGTCCTTCCCCAGTTCAGTCCC |
| | | CACTCACCTGTTCACCCTTATCTCCTACACTCGCTGGGTGCTC |
| | | TCTCCATTCCAGCCACAGGGCCTCCTTGCTGGCTTTCAACCT |
| | | CCTAGGCATGCTTCTGCCTCCAGGCCTTTGCACCGGCTGTGC |
| | | CCTCTGCCTGGAATACTTCTCCCCAGATATCTGCATGGCTCA |
| | | TGCCTTCACCTCCTCTGGTCTTTACTCAGTGAGGCCTTTTCTT |
| | | TTCTTTCTTTCCCTGTTTCTTTCTTTCTTTCTTTCCCTCCTTTCT |
| | | TTCTTTCTTTCCCTTTCTTTCTTTCTTTTTCCTTTTTTCTTTTTC |
| | | TTTTTTTGAGGTGGAGTCTCGCTCTGTCACCCAGGCTGGAGT |
| | | GCAGTGGCACAATCTCGACTCACTGCAACCTCCTCCTCCCAA |
| | | GTCCAAGTGATTCTCTTGCCTCAGCCTCCCGAGTAGCTGGGA |
| | | CTACAGGCACATGTCACCACATCTGGCTAATTTTTTGTATTTT |
| | | TAGTAGAGACGGGGTTTCACCGTGTTAGCCAGGATGGTCTCA |
| | | ATCTTCTGACCTCGTGATCCCCCCACCTCGGCCTCCCAAAGT |
| | | GCTGGGATTACAGGCGTGAGCCACCACACCCGGCCTCAGTG |
| | | AGGTCTTTTCTGACCCTCACAGTTAAAAATAAAACTCTGGCC |
| | | AGGCGCAGTGGCTTATGCCTGTAATCCCAGCACTTTGGGAGG |
| | | CCGAGGCAGGTGGATCACAAGGCCAGGAGTTTGAGACCAGC |
| | | CTGGCCAACATAGTGAAACCCCATCTCTACTAAATATACAAA |
| | | ACAAATTAGCTGGGCATGGTGGCAGGTGCCTGTAGTCACAG |
| | | CTACTTGGGAGGCTGAGGCAGGAGAATCATTTGAAACCGGG |
| | | AGGCGGAGGTTGCGGTGAGCTGAGATCACGCCACTGCAGTC |
| | | CAGCTTGGATGACAGAGTGAGACCCCATCTCAAAAATAAAT |
| | | AAATAAATAAATAAATAGAACTCCATTCCCCTCATGGTTAAA |
| | | AAAAAAACTCCATCCCCCTCACCACTGTATATTTCCATATGA |
| | | TTCATTGTCTCAGACATCAGCTTGCTTTCCGATTTCTTTGATT |
| | | ATCTGTCTATCTTCCCCCCTGGAATGTCAGCACCCAGAAGGC |
| | | AGGGATTTTCGTCTGTTTGGCCCACAACTTTCCTCCCTGGGC |
| | | CTGGCACATAGTAGGCAAGCGGTCAATATTTAACAACGAAT |
| | | GCCTTAATCCAAGAACCCCCATAAGATTGGGGTGAAATATTT |
| | | GAGGAGATCAAGACTTAAGGGTGAAGGGATTTGCCAAGGCC |
| | | TTTAGCAGGGAGCCAAATGCCAGATCCTGGAGTCAAACTCT |
| | | GATCCTGTGGCCTTTTGCTGAGGCCAGTGTTCTCAGACTTCA |
| | | GGATTTCAGAGGCCGAGAACATTTCGGAATGAATTCGAGTG |
| | | ACCGTGCGCGGTTACTGTTTTTTAATGTTTATCAAGGATGCT |
| | | CATTCAAACAAACAAATAACAACAAAATACCCACTGCTGGC |
| | | ACCATAATTTTACCAAAGAATGATCATTCTAATATTGAAAAA |
| | | GTTCAAAGGACATAATTGCAGAATAAAAGACAGCTCTTTAT |
| | | ATGGAATGCATTTAACCATATTTTTAAACTAAACGGCCCTT |
| | | CGTTGTCCTTATTTTTCTCATCCCCTTGAAGTCTGCTCTAAAT |
| | | GTTGATCCAGGCTGTGTGACCCTGTGTGAGCTATTTGATCGC |
| | | CTAGTGCCTCAGTTTCCCTATATGTAAAATGGAGATAAGAGC |
| | | CCATAGCTCAGAGTCTTGAGGAGTAAATGAGATCTAGGCCG |
| | | AGGGAAGGGCCTAGCATGCCCCTGGCTTATAATAAATGCTC |
| | | AACAAATGGTTCCTTTTGTTATTAAAATGCAAAGGCATATTA |
| | | GTCAACCACCTGGTTTACCACTGGCAGTGGTCGTCAGCTGGC |
| | | ATTTCAGGACCCCTTCCCCAGGCTGCGGCGGGAGAAGGGTA |
| | | TCTGTGGATGTTGGGAAGAGTAAGAACTGGCTCCCTACTTAG |
| | | AGACAAAAGCTGGGGAGCCCCAAGATGGGAGCCCAACTTAC |
| | | CTCTTTTTCTTTTCTTTTTTTGTTTTTTTTCTTTTTTGAGACAG |
| | | GGTCTCGCTCTTACCCAGGCTGGAGTGCAGTGGTGTGATCAT |
| | | GGCTCACTGCATCCTCCACCTCCTGGGCTCAAGCGATGCTTC |
| | | CACCTCAGCCTCCTAAGAAGCTGAGACCACAGGACACACCA |
| | | CCACACTCGGCTAATTATTGTGTTTTTTGTAGAGACTGAGTC |
| | | TCACTATGTTGCCCAAGCTGGTCTTGAACTCCTGGGCTCAAG |
| | | CAGTCCTCTTACCTTGGCCTCCCAAAATGCTGGAATTACAGA |
| | | CATGAGCCACTGTTCCCAGCCTCAACTGGCCTCTTATGGGGG |
| | | GCTATGAAATCTACCCCAAGCAGAAGAGATTGGGGTCCTAC |
| | | GTGGAAGGGTTGCAGGGACCTAAGAGGCTGGGGTGGAAGG |
| | | GAAGAGGAGGGGCCTCAGACAGGACCTGACTTCTGTGCCTT |
| | | GTCTCTGCCCCACCCTGGGGCTGAATGAGGGGCTGTGACTGT |
| | | GACTGGAGGTCTTACCCTTGCTCTTTGCCTCCTAGGCTGGGC |
| | | CCTGTCTCAGGCCCCCAGGCTTTCCCCAAACTGGTGCGGATC |
| | | CTCACGGCCTTTTCCTCCCTGCAGCATCTGGAGTGAGTATAG |
| | | ACTCTGGGACCCCTTCCTCTCAACATCTGGGTGCAGTGCTGT |
| | | GATCATAGCTCACTGCAGCCTCGAATTCCTGGCCTCAAGTAG |
| | | TCCTCCTGCCTCAGCCTCCTGAGTAGCTGGGACCACAGGTGC |
| | | ATGCTACAGTGCCCAGCAACCTCTGGGTTTTGACAACCCCAA |
| | | ATCCTGCCCCCAAGGCACCTGCAGAAAGAAAAGCCCTCACT |
| | | TTGTCCCTTTTTTTCTCTCTTCCGCCTACTGCATCCTCAATGT |
| | | GGCTGGCACATGAGTGATGAGATGACGCCACATCAGATGGG |
| | | AATCCCCAGTGACAAAAAAAGTTGTAGATCCTTTCGTGACCT |
| | | TTGAAACTCCTTTCCTCACAACCTCATTTGCTGCTCTCAGCAA |
| | | TTCAGCTGGGGAGGGATTGGGGAGCATAGTCCTACTTTACA |
| | | GTGGAGGTTAAGGAAGTTGCCAAGCCTCAGGGGCTTAGTTC |
| | | CTAAGAAGCTAAACAGGGATTCAAGTCCATCATGAGTTAGA |
| | | CTTTGCCCTCTGGCTGCCTGGTTGGAGCCCCCAGGTGATGAA |
| | | GTCCTCGTCTGAAAAGCTCAGGAATGTGCCGGCTGCCTATGG |
| | | TGTATTAGAGCTGGGGGGTGGGAAGGGCAGGTGCCAGGGCT |
| | | GAGGAGGCCCTCACTGGGATGGGAAGGGTCAGATGGCCCCA |
| | | GGACGCTAGCTGATGGCCCCCATCTGATTCCACCTGCAGCCT |
| | | GGATGCGCTGAGTGAGAACAAGATCGGGGACGAGGGTGTCT |
| | | CGCAGCTCTCAGCCACCTTCCCCCAGCTGAAGTCCTTGGAAA |
| | | CCCTCAAGTGAGTGAGCTGGGCCTGCCCTTCCTGCTGAATCG |
| | | GGCCCCCAAAGTCCGGCTGACTTTTTCAAAATTAATTTAAAT |
| | | TTGTTTTTTTAGACAAGGGCTCGCTGTGTCACCCAGGCTAGA |
| | | ATATAGTGCTATGATCATACCTCTGCAGCCTTGAACTCCTGG |
| | | CCTCAAGGAATCTCCTCACCTCCGCTTCCCAAAGTGCTGGGA |
| | | TTATAGGTGTGACTTACCATGCCCAGCCCCAGTGGATTCATC |
| | | CATTCACTCATTCACCTATTCAATCAGCCATTCATCCTTCCAT |
| | | TCAATGATTCATCCATTCACTCATGCAGTCATTTCTTTACTCC |
| | | CTCATCCAACCACTCACTCAATCATTTCTTTACTTACCCAACC |
| | | ATTTATTAATTTAATCAACAAATATTTAAGGAGGACCTACTA |
| | | TGTGCAGGCACCTTTCTAGGCCCTGGAGACCCAGCTTTAAAC |
| | | AAAACAAATAAATCCCCTGACCTCAGGAATCTTAAATTCTAG |
| | | TGAGGAGACACAATGAACAAGTAAACAAGTCGGTAATTATA |
| | | AAGTATACAGTAATGAAAATAATGTTTGGTGACAGAAAATT |
| | | ATACTGGGGGGTGAAATAGGAGAGATAGATTAGGATTTATC |
| | | AATAAAGCATTATTATCAAAACAGTAAAACTTTTTAAAAACT |
| | | GTTCTGGGCTCTGGGAAGTTGGAGATGGATAAGATACAGGA |
| | | GGATCAATGTGATTTTTTTTCTTTTTTTCAGACAAGGTCTCGC |
| | | TCTGTCACCCAGGGTGGAGTGCAGTGGTGTAATCATGGCTCA |
| | | CTGCAGCCTCAATCTCCTAGGCTCAAGCAATCCTCCCATCTC |
| | | AACCTTCTGAGTAGCTGGGACAGGTGCATGCCACCACGTCCC |
| | | GCTAATTTTTGTATTTTTTTGTAGAGACGTGGTTTCACCACGT |
| | | TGCCCAGGCTGGTCTTGAACTTCTGAGCTCAAGCCATCCTCC |
| | | TGCCTCTGCCTCCCAAAGTACTGGGATTACAGGCATGAGTCA |
| | | CTGTGCCTGGTTCAATGGGATTTTTTTTTTTTTTTTTTGACAC |
| | | GGAGTCTCGCTCTGTAGCCCAGGCTGGAGTGCAGTGGTACG |
| | | ATCTATTTATTGCAAACTTCACCTCCTGGGTTCAAGCAATTCT |
| | | CACACCTCAGCCTCCCAAGTAGCTGGAATTACAGGCATGCA |
| | | CCACTACACCCAACTAATTTTTGTATTTTTAGTAGAGACGGG |
| | | GTTTCACCAGGTTGGCCAGGCTGGTCTTGAACTCCTGACCTC |
| | | AAGTGATCCGCCCACCTCAGCCTCCCAAAGTGCTGGGATTAC |
| | | CGGCATGAGCCACCACGCCCAGTTCAACATGATTTTTTACTG |
| | | AACCTAGAGGATATCAAAATCTTCCCACACTCCCCCTCACAA |
| | | CCAAGGTCTTAGGATGGCCAAATAATTTACCGGAATTTACTT |
| | | AGCAATTTCCTTAATTGTAGGACTGTGGGACTTCTCCAGTAA |
| | | TTTCTAGTGTCCTAGGTAACACTGTAAGCTAGATTTCTTCTCC |
| | | TTAGTCCTTCCGTCCCCCTCTTTCTTACCCGCCACCCCACTTA |
| | | AGTATTTATTGAGCACCTACTATGTGCCTGGCAGTGCTCTAG |
| | | GCACAGGGATACAGTAATGAGCAAAACAGACACAGCCCTGC |
| | | TCCATGTGAAGCCCACAACCCGGCAGAAAGGATTACACAGC |
| | | CATTAAACAAATAATGCCAACACTTATTTCATTATAGTGCTG |
| | | AAAAGTGGCGCAGAGGGAAAAGAGGGGATGTTGTAAAGTC |
| | | ATGGGACAGGTGGGGGCAGGAGGGTGCACTCTCCCTCCCAT |
| | | CCACCTCCTCATCCCTGCCCTCCCACTTGGCCACAGGACCTA |
| | | ACAGTATCCTCCTATTTACCGAGAGGTAGCTTAAGTCTGTCG |
| | | ACAGCGCCATTCACAGCCTATGACCCAGAACTCTCCTTGAGG |
| | | TCAAAGTGAGGCATGCAAGTTTGGTCCTGAGCCCTCCCCCTC |
| | | ACTGTGTCCCCGCAGTCTGTCCCAGAACAACATCACTGACCT |
| | | GGGTGCCTACAAACTCGCCGAGGCCCTGCCTTCGCTCGCTGC |
| | | ATCCCTGCTCAGGCTAAGGTGAGTGGGGCCCCGGATACCGG |
| | | TCAGGTGCTGAGCTGGGGGGCTGCAGAGCGCAGGGAACCCA |
| | | CATCGTGCTGGCTGCAGGGGACACTGAGACCCTAGAAGCAA |
| | | TCACCACAGCCCTGAACAAAAGGATTAGCGGGACGTGGTGA |
| | | AAGAAACTCTGAGCAAGTCACTTATTCATTCCTAGCCCCTGT |
| | | TTTCTTACCTGGAAAATGGGAACCATCCTAATGGTCTATGTA |
| | | AGCACTTGTCACAATGCCAGGTTCTGTTATGCAATAAATATT |
| | | GCTCCTTTTCCCCCAGGGATGGCGGGACCAGGCTTTTTCTGG |
| | | AACCTAGGGGTGGTGGCTTCTGGAAGGCTAACCACGCACGT |
| | | CAGCTTTTGCCGGCCTTGTCACTTACATGCCGGTCAGTGTTTC |
| | | ACTGCCACCTTCTGGTAGGCCTTGGCATAGCACCTCTTGCTT |
| | | TTGAGTGACTTCCATCCAGGCCCTGGTGTGGTCCCAGAGGTA |
| | | ATGAGCGGACTGAGCTTGGACCAGAGCCCCAGTCATCCAAC |
| | | TCAGCATTACCAGCTCTTCTTACTCGCCAGACTGAGAGAAAC |
| | | TGCTCTCCTCTTCGCCAGAGCTCTTCACTTACTGAGGACAAC |
| | | CAGAGTCAGATTCCCAGGAAAGATGGCATTTGGTCCCTTATT |
| | | ATTTATTTATTTTTAATTTTTTTAATTTCTGTTTTTGAGATGGA |
| | | GTCTCGCTCTGTCACCCAGGCTGGAGTGAAGTGGTGTGATCT |
| | | CAGCTCACTGCAACTTCTGCCCCCCCCCAGGTTCAAGCTATT |
| | | CGCCTGCCTCAGTGCTCCGAGTAGCTGGGATTATAGGCGCCC |
| | | ACCACCACGTCCAGCTAGTTTTTATATTTTTAGTAGAGACAG |
| | | GGTTTCGCCATGTTGGCCAGGCTGGTCTCAAACTCCTGATCT |
| | | CAGGTGATCCACCTGCCTCGGCCTCCCAAAGTGAGTACTAGG |
| | | ATTACAGGCGTGAGCCACCATGCCCGGCCAACTTATTTATTT |
| | | ATTTATTTATTTATTTATTTGGAGATGGGGTCTCACCCAGGTT |
| | | GTAGTGCAGGGGTGCAATGATGGCTCACTACAGCCTCGACT |
| | | ACCTGGACTGAAGCGAACCTCCCACCTCAGCCTTCCAAGTAG |
| | | CTGGGTCCTATACCCTCCTGAGCCTTGTTAATTTCTTCCCAGC |
| | | ATGATCACTGTTTTACAATATTTTATTTATGTAGGTGCTTACT |
| | | TCTTGCTATGCAAAGAAACTCAGAAGCAAAAATAGAATCTG |
| | | TCTGGTTCATAATTGTGCTAAAAACTAGAGATACAAGTATGA |
| | | ACTAGACAGATGTCGTTCTTGCTTTTGAGTTTCTTTGCGTAGT |
| | | AGGAATTAGGCACATACATAAATAAAATATTGTAAAACAGT |
| | | GATTCATGCTGGGAAGGAAACTAACAAGCCCCAGGAGGGTA |
| | | CAGGACTTCTGGGGAGATGATTCTTCCGTGGGAAGCTCTAGG |
| | | GGAAAAGGACTCTAGGAAGAAAGAACAGCAGGTGCAAAGG |
| | | CCCTAAGTGGGAAAAGGGGCTGTGTTTTAAGGGTCAGAAGG |
| | | GCCACTGTGGCTGTGTGTAGTGAATGAGGGACAGAGGCTGG |
| | | GCAATGGTGGAAGTAGGGGAGGTCAGGGGAATCCAGATCAC |
| | | ACAGGGAGGCCCTTGTGGGTCACAAGAGGTATTTGCAGTTTT |
| | | TCAGTGTGAATGGAAAAGTAGGTGGGCCTGGGAAGGCGACA |
| | | CTTGATCTGATTTACACTTTTCTTTTTTCTTTTCTTTTCTTTTT |
| | | GAGACCAAGTCTCACTTTATTGCCCAGGCTAAGGTGCAGTGG |
| | | CATGATCTCAGCTCACTGCAAGCACTGCCTCCTGGGTTCAAG |
| | | TGATTCTCATGCCTCAGCCTCCCAAGTAGCTGGGATTACAGA |
| | | TGCCCGCCACCACACCAGGCTATTTTTGTGTTTTCAGTAGAG |
| | | ACAGGGCTTCGCCATGTGGCCCAGACTGGTCTTGAAATCCTG |
| | | GGCCCAAGCGATCCACCCACCTCAGCCTTCCAAAGTGCTGG |
| | | GATTACGGGTGTGAGCCACCATGCCCAGCCTGATTTACCCTT |
| | | TAAGAAACATCATATTTCATGGAATCTAAGATGCCATCCATT |
| | | GTAAGACCCCAATATGAGGCCAAAATGCAAACTGTGACACG |
| | | CTATTGGTTATAAGACACATCCTGATCTCAATGATATTCAAA |
| | | TGGTGACATATATGTCTTGGCATCGATGAAATATGGTCAGAT |
| | | TACTCTCCCTGTGGGGTGGAGGAAGGGTCTCCAGGGTTCAG |
| | | AGATTGCAAAACCCCCATCTAGTTTCTGCACTGGTACCGGCC |
| | | GACCCGTGATGTGAGTTGATTTAGCCTGAATCCACTTGGCAG |
| | | CAGTGTAAACCTGCTGCATTTATTTATTTTTTTCTTTTTTTTTC |
| | | GAGACAGAGTTTCGCTCTTGTTGCCAAGGCTGGAGTGCAGTC |
| | | TCAGTTCACTGCAACTCCGCCTCCCGAGTTCAAGCAATTCTC |
| | | CTGCCTCAGCCTCTCAAGTAGCTGGGAGTATAGGCATGTGCC |
| | | ATCACACCCGGCTAATTTTGTATTTTTAGTAGAGACGGGGTT |
| | | TCTCCATGTTGGTCAGGCTGGTCTCAAACTCCCGACCTTAGG |
| | | TGATCCACCCGCCTCGGCCTCCAAAGTGTTAGGATTACAGGC |
| | | GTGAGCCACTGCACCTGCTGCTTTTATATGCAGCCCTGCAAG |
| | | GGAGTTCTGAGGCTGAATCATGGCAAAGTCATGGCCCTGAA |
| | | ATCATACCCGCAGCAGCACCTGACCTGTTAAAAGGCTGCTTG |
| | | GTGTCGAGTAAACAGCTGAAGTTGGCATCAGTGACACTCAT |
| | | CATGCTATTTTCACTGCTACTAATGCTGGTTGTGAATATGCT |
| | | GCAGAAACTGGAGCTGGTCCAGTGACTGGCTTTTTCTCTTCC |
| | | TGCCCCCAGTGCTGGTCACACCCTCTATGTTAACTCATGCGA |
| | | CCCTCACCAATGTTCCTGTGATGTAGTCAGAACCATAGCTAA |
| | | CATTTAGTTCATTCTAGGCACCAAGGCATATTCTAAATGTTA |
| | | TATCAGCATTGATTCATTTACTCCTCCCCACAATGACTCTATC |
| | | ATGCCCATTTTACAGATGGGCAAACTGAGTCTCAGAAGAGTT |
| | | GAGTAGCTTGCCCAAGGTCACATAGGCAGTAAGAGGCATAG |
| | | CTGGGATTTGAACTTGGGAAGTTGTTCTGACAGTTTCCACAC |
| | | AGTAAGGCAAGAAATGACTTCCTTTTAGTCCTGCTGTTTTCC |
| | | TGGGCTCACTGTTTGACCACAGAGAAGTGGTCTCAACTCTCT |
| | | GGGTCTTGGTTTGCCTGTCTTTAAAATGGGGCAGTAATCCCA |
| | | GTAGGTGGTGACTCCCACGGGGCTGACCTCCAGGCTGCAGG |
| | | GGACACTGAGATCCTAGAAGCAATCACCACAGCCCTGAACA |
| | | AGAGAACTAGTGGGACCTGGGGACAGAAACTTTGAGCAAGT |
| | | CACTTATTCATTCCTAGCCTCTATTTTCATATCTAGAAAATGG |
| | | GAACCATCCCAATGGCCTATGTAAGTACTTGGCACAATGCCA |
| | | GGCTCTGTTGTGCAATAAATATTGGTTCCTTCCCCCAGGGAT |
| | | AGTGGGACCAGGCTTTTTCTGGAATCTAGGGATGGTGGCTTC |
| | | TGGAAGGCTGACCATGCACAGGCCTCCAATCCCTCCCCCTGG |
| | | CCTCTGTTTCCGACAGCTTGTACAATAACTGCATCTGCGACG |
| | | TGGGAGCCGAGAGCTTGGCTCGTGTGCTTCCGGACATGGTGT |
| | | CCCTCCGGGTGATGGAGTGAGTGTGGGAGTCTGGGCGGTGG |
| | | GTGGCTCAGCCCGGGGTGGGAGACACTGAAGTCTCTCCCTG |
| | | GTGTCCTGGAAGAGCTGGATGTGGGGGTGGCCTTGGTCTGG |
| | | AGCTGGGGAGTCCCAAGGGCCAGGCCCCAAGGTGAGTTTCT |
| | | CTTGCCAGCGTCCAGTACAACAAGTTCACGGCTGCCGGGGC |
| | | CCAGCAGCTCGCTGCCAGCCTTCGGAGGTGTCCTCATGTGGA |
| | | GACGCTGGCGTAAGTCCAGGCAACCCTGGTGGGTGGAGAAC |
| | | AACTCACTCCCCAGGCGTGTGGCCCAGTGTGACCCCGGAGCT |
| | | CAAATCATGCTCTTCCCTTCACCAATCTGCAACCCTGGGTGA |
| | | GCAGACACTTCCCCCTCTGGCTTCAGCTTCTTCATCTGTAAA |
| | | GTGGGGACAGTAATAATATCCACCCAAGAGGTTTTGTGAGG |
| | | CTTAAACTAATCAAGCCCTACAAAGCTCTTATAACAACAGTA |
| | | GCTGGCACACAAAAATGCCAATAAATGCTATATTAGGCTGA |
| | | ACCATATGAAATTGCTGACATCCACCCATTTTTGACCTGTAA |
| | | AATTGGCAATTTCATATGGTTCAGGTTTATCTTTTATTAAGTC |
| | | ACCATGATGTCCAATACTGGTTACTTTATATTCTTTCAAAGC |
| | | ACTTTTATCTCCACAATCACTCACCTCTTCTAAGACGCACAG |
| | | GGTGGGTCTTACAAGACCCATTTTACAGATGAGGAAAATAA |
| | | AGCACAGAGCAGTTAACTAACCTTTCTGGGGTCACACAGCA |
| | | AGTCAGCTGCAGAACCATAAAGGAATCTCGGGCCTCCTAGG |
| | | CTTCTCCTTTTCCCCATGACCCACACCGGTCGGTATCTTGCCA |
| | | GGGGAAAAGTCCCCAACGTTCTAGGCTGGGTGGAAGGAGGG |
| | | ATTTGGGGGCAGCTGTCACTGGGGCCCCAGGCCGCCCTCTCT |
| | | CCTCTAACCTGGCTCTGAGTCCCATCCCCCCTTGCAGGATGT |
| | | GGACGCCCACCATCCCATTCAGTGTCCAGGAACACCTGCAA |
| | | CAACAGGATTCACGGATCAGCCTGAGATGATCCCAGCTGTG |
| | | CTCTGGACAGGGTAACCAGGGTGGGCTTGGGAGGGGAGAGC |
| | | CGCAGTGGGTTGGGGGCAGTGTCCTTGTGAAGGTGGCATTC |
| | | AAAAAATGTGGGCGGGACACAGGTGGGGCTAGGCCACCACC |
| | | CTTGGACGCATGCGTCATCAGAGACATCCCCTCATCTCCACC |
| | | CTGGGCTCGGTGGAGCTGTCCTCCAGGCTTTGCGAGCTTGGT |
| | | CCCTGTGGTCAGGCTTAGAGATCAGGGGACAGGAAAGTTGG |
| | | CCATCCACATCCCACAGCCTCAGTGCTTGGAAGAGCTTCCTT |
| | | TGGGGACTCCAAGCCTTCCCAGCTGCTGTTTCGGCTTGGTGG |
| | | CTGCCCTGATGCTCCGGGTTTGTCTCAGATGAACTTGCTTGA |
| | | CAAGTCTCCTGCTCCTCACTATGAAGATCACTGTCCCCCAGC |
| | | CCTGTGCTCCCCGCACTGTGCTGCACGTCCACCTCCATTCCA |
| | | CTGCCCCTCCCATCCCCCCATCTTGATAGCACCCTTCCCAGG |
| | | TGTCAAGCTGCCCCTCCTAGAGTGTCCTGCCTAAACCCCCTC |
| | | TCCTGGCTCCTCCCGCTACAGCATGTTCTCTGAGGACACTAA |
| | | CCACGCTGGACCTTGAACTGGGTACTTGTGGACACAGCTCTT |
| | | CTCCAGGCTGTATCCCATGAGCCTCAGCATCCTGGCACCCGG |
| | | CCCCTGCTGGTTCAGGGTTGGCCCCTGCCCGGCTGCGGAATG |
| | | AACCACATCTTGCTCTGCTGACAGACACAGGCCCGGCTCCAG |
| | | GCTCCTTTAGCGCCCAGTTGGGTGGATGCCTGGTGGCAGCTG |
| | | CGGTCCACCCAGGAGCCCCGAGGCCTTCTCTGAAGGACATT |
| | | GCGGACAGCCACGGCCAGGCCAGAGGGAGTGACAGAGGCA |
| | | GCCCCATTCTGCCTGCCCAGGCCCCTGCCACCCTGGGGAGAA |
| | | AGTACTTCTTTTTTTTTATTTTTAGACAGAGTCTCACTGTTGC |
| | | CCAGGCTGGCGTGCAGTGGTGCGATCTGGGTTCACTGCAACC |
| | | TCCGCCTCTTGGGTTCAAGCGATTCTTCTGCTTCAGCCTCCCG |
| | | AGTAGCTGGGACTACAGGCACCCACCATCATGTCTGGCTAAT |
| | | TTTTCATTTTTAGTAGAGACAGGGTTTTGCCATGTTGGCCAG |
| | | GCTGGTCTCAAACTCTTGACCTCAGGTGATCCACCCACCTCA |
| | | GCCTCCCAAAGTGCTGGGATTACAAGCGTGAGCCACTGCAC |
| | | CGGGCCACAGAGAAAGTACTTCTCCACCCTGCTCTCCGACCA |
| | | GACACCTTGACAGGGCACACCGGGCACTCAGAAGACACTGA |
| | | TGGGCAACCCCCAGCCTGCTAATTCCCCAGATTGCAACAGGC |
| | | TGGGCTTCAGTGGCAGCTGCTTTTGTCTATGGGACTCAATGC |
| | | ACTGACATTGTTGGCCAAAGCCAAAGCTAGGCCTGGCCAGA |
| | | TGCACCAGCCCTTAGCAGGGAAACAGCTAACGGGACACTAA |
| | | TGGGGCGGTGAGAGGGGAACAGACTGGAAGCACAGCTTCAT |
| | | TTCCTGTGTCTTTTTTCACTACATTATAAATGTCTCTTTAATG |
| | | TCACAGGCAGGTCCAGGGTTTGAGTTCATACCCTGTTACCAT |
| | | TTTGGGGTACCCACTGCTCTGGTTATCTAATATGTAACAAGC |
| | | CACCCCAAATCATAGTGGCTTAAAACAACACTCACATTTATT |
| | | CTGCTCACATATCTGTCATTTGAGCAGGGCTCAGCGGGGACA |
| | | GCTCCTTCTGTCCTACTCTGTGTCAGGTGGGGCAGCTTGAGG |
| | | GTTGGGCTGGTGTCACCTGAAGACTCATTCTTCTGTACGTCT |
| | | GACAGGCAATGCTGGCTGTTGGCTGGGGGCCTCAGTGCCAC |
| | | TACGGAATAGTTGGCTAGGACCCCTCCATGTGGGCTAGTTGG |
| | | GCTTCCTCATAGTATGGTGGCTGGGTTGGAGGGTGTCCCAAA |
| | | AAGAAAGGAGGGGATAGAGAGAGACCACTTTTCATAACCTA |
| | | GCCTTAGAAGTCACACAGTATTACTTCTGCTACATATATATG |
| | | TTTTAAGAGGCAGGGTCTCACTCTGTCGCCCAGTCTGGAATG |
| | | CAGTGGTATGATCACGGCTCACTGCAGCCTCAACCTCCTGGG |
| | | CTAAGTGATCCTCCCACCTCAGCCTCCCGAATAGCTGGGACT |
| | | ACAGGTGTGAGTCACCAAGCCCAGTTAATCTTTAGTTTTATT |
| | | TTTGTAGAGCCAGGGTCTCACTATGTTGCCCAGGCAGGTCTT |
| | | GAACTCCTGGCCTCAAGTGATTCTCCTGCCTCAGCCTCCCAA |
| | | AGTGCTGGGATTACAGGTGTGAACCACCACACCCAGCCCAC |
| | | TTCTGCCATATTCTGTTGGCCAGTGTGACAAGGATTGCTACT |
| | | GTCCTACCCACCCTCCTTTCACCACATGTGCACATGCACGTG |
| | | TGTGCACGTACACACACATACACACACACGCGTGCACACAC |
| | | CAGAGCCCACCTTGGCTCAAGTCCTCTTTTCTGAGAGGACTT |
| | | TTCTTTGTGGCTTCCTAAAATTCAGTGGAAAATTAATTGTTTG |
| | | GGGATGAGAAAGGTTGAAGCACCAAAAGCTTACCAAGGGG |
| | | AATGTTTGCCTCTGCCTCTGACACACAGCTCTGTCCTGGGAG |
| | | TGAGCTGGTTTTTAGCGGAGACGGAGTCCCACCTTGGCTGCA |
| | | GGGAGTCCGAAAGGGACTTGGAAGCTCCGCTTTCTACCCAG |
| | | CGAGCTGCCGGCACGACTGTTGCTTTTCACTCGGGCATAGTC |
| | | TGCTCAGAAGCCCCAATTCAAGACATCTTAGCTTACTCCTGG |
| | | TGGCAGTGGGAGCTGCCTGTTCAGCTCCAGCTCACCAGCCCC |
| | | AGTGCCCACAGGATCAGTCTGATTCCCAAGCTCTGCTCCTCT |
| | | CCCCAGCAAGTGAGAGCTGGGTGTCAAGAGGGTCTGAGGAA |
| | | TCCAGACCCGGCTGCGTGGTTGATGATCTTTGCAGACAGGCA |
| | | AGCACCTCCTGCCTCGAACTGGCTGCAAAGGGTATCAGGTG |
| | | CTGCCATCCAGGTTCAGCTTGTAAATAGCTCAGGTGTCACCC |
| | | TGCAAGGGATCCCTGCACTTGCAGACTCTACAGAGGCCATG |
| | | GGCCTCCTGTGTGTGCGGCTACAGGAGTGAGCACTGAGGTG |
| | | TGCTCTGATCATCCACTGTGCCATGTGCCAGGTTTTTGGTCTG |
| | | ACCCACTGCTTTTGGTCTGTGTGATGATGAAATGAGGTCAGA |
| | | GCCTGCAGTTATTAACACTAACGATTTGTTAATGATAGCTAC |
| | | TGTTTATTATTTTATTTAATTTTTTTGAGACAGAGTTTTCACT |
| | | CTTATTGCCCACGCTGGAGTGCAGTGGCATGATCTCGGCTCA |
| | | CTGCAACCTCTGCCTCCTGGGTTCAAGCAATTCTCCCACCTC |
| | | AGCCTCCCAAGTAGCTGGGATTACAGGCATGCACCACCACG |
| | | CCTGGCTAATTTTTGTATTTTTAGTAGAGATGGAGTTTCACC |
| | | ATGTTGGCCAGGCTGGTAATCTGCCTGCCTTGGCCTCCCAAA |
| | | GTGCTGGGATTACAGGGGTGAGCCACCGCACCCGGCCACTA |
| | | CTGTTTATTTAATGAGCAGATACATGCAAAGCCCTTATCACA |
| | | GTGCAAGGGAAATTCAAAGCGCTCAGAAAGTATTAGCTCAA |
| | | TAAGTGATGACTGTGTGCCAGACACTGTGCTAAACTCCTACT |
| | | CAAGAGGGATAAGAGTCTAGGGGCAAGTGGCAG |

Gene manipulations and modifications to a mammalian cell or an engineered mammalian cell may be carried out using any known method in the art, including gene silencing, gene knock downs, gene knock outs, and gene editing techniques. For example, a gene mutant may be generated using a targeted genome editing technique at a desired site(s) in the target OCRs. The targeted genome editing technique may be any technique known in the art, e.g., techniques that employ site directed nucleases such as CRISPR-Cas, zinc finger nucleases, transcription activator-like effector nucleases (TALENs), and meganucleases.

The engineered mammalian cells described herein may be derived from a variety of different mammalian cell types (e.g., human cells), including adipose cells, epidermal cells, epithelial cells, endothelial cells, fibroblast cells, embryonic stem cells, induced pluripotent stem cells, mesenchymal stem cells, pericytes, keratinocyte cells, subtypes of any of the foregoing and cells derived from any of the foregoing. Exemplary cell types include the cell types recited in WO 2017/075631. In some embodiments, the cells are derived from a cell-line shown in Table 2 below.

TABLE 2
Exemplary cell lines

| | | Germ | |
|---|---|---|---|
| Cell Line | Cell Type | Layer | Commercial Source |
| ARPE-19 | Epithelial (Retinal) | Ectoderm | ATCC (CRL-2302) |
| BJ | Fibroblast (Foreskin) | Ectoderm | ATCC (CRL-2522) |
| CCD-841- | Epithelial (Colon) | Endoderm | ATCC (CRL-1790) |
| CoN | | | |
| HaCat | Keratinocyte | Ectoderm | Addexbio (T0020001) |
| HHSEC | Endothelial (Hepatic Sinusoidal) | Endoderm | Sciencellonline.com (#5000) |
| Huv-EC-C | Endothelial (Embryonic umbilical) | Mesoderm | ATCC (CRL-1730) |
| MCF-10A | Epithelial (Mammary Gland) | Ectoderm | ATCC (CRL-10317) |
| MRC-5 | Fibroblast (Lung) | Mesoderm | ATCC (CCL-171) |
| MSC, human | Mesenchyme (Bone Marrow) | Mesoderm | ATCC (PCS-500-012) |
| MSC, mouse | Mesenchyme (Bone Marrow) | Mesoderm | Cyagen (MU BMX-01001) |
| WS-1 | Fibroblast (Skin) | Ectoderm | ATCC (CRL-1502) |
| 293F | Epithelial (Embryonic Kidney) | Mesoderm | Thermo Fisher (R790007) |

In an embodiment, any of the engineered mammalian cells described herein is derived from an RPE cell, e.g., an ARPE-19 cell. In an embodiment, an engineered RPE cell (e.g., an engineered ARPE-19 cell) comprises any of the expression cassettes, transposons and polynucleotides described herein.

Engineered mammalian cells for use in devices, compositions and methods described herein, e.g., as a plurality of engineered cells contained or encapsulated in a hydrogel capsule, may be in various stages of the cell cycle. In some embodiments, at least one engineered cell in the plurality of engineered cells is undergoing cell division. Cell division may be measured using any known method in the art, e.g., as described in DeFazio A et al (1987) J Histochem Cytochem 35:571-577 and Dolbeare F et al (1983) Proc Natl Acad Sci USA 80:5573-5577, each of which is incorporated by reference in its entirety. In an embodiment at least 1, 2, 3, 4, 5, 10, or 20% of the cells are undergoing cell division, e.g., as determined by 5-ethynyl-2′deoxyuridine (EdU) assay or 5-bromo-2′-deoxyuridine (BrdU) assay. In some embodiments, cell proliferation is visualized or quantified by microscopy (e.g., fluorescence microscopy (e.g., time-lapse or evaluation of spindle formation) or flow cytometry. In some embodiments, none of the engineered cells in the plurality of engineered cells are undergoing cell division and are quiescent. In an embodiment, less than 1, 2, 3, 4, 5, 10, or 20% of the cells are undergoing cell division, 5-ethynyl-2′deoxyuridine (EdU) assay, 5-bromo-2′-deoxyuridine (BrdU) assay, microscopy (e.g., fluorescence microscopy (e.g., time-lapse or evaluation of spindle formation), or flow cytometry.

In an embodiment, at least 50%, 60%, 70%, 80%, 90% or more of the engineered cells in the plurality are viable. Cell viability may be measured using any known method in the art, e.g., as described in Riss, T. et al (2013) “Cell Viability Assays” in Assay Guidance Manual (Sittapalam, G. S. et al, eds). For example, cell viability may be measured or quantified by an ATP assay, 5-ethynyl-2′deoxyuridine (EdU) assay, 5-bromo-2′-deoxyuridine (BrdU) assay. In some embodiments, cell viability is visualized or quantified by microscopy (e.g., fluorescence microscopy (e.g., time-lapse or evaluation of spindle formation) or flow cytometry. In an embodiment, at least 80% of the engineered cells in the plurality are viable, e.g., as determined by an ATP assay, a 5-ethynyl-2′deoxyuridine (EdU) assay, a 5-bromo-2′-deoxyuridine (BrdU) assay, microscopy (e.g., fluorescence microscopy (e.g., time-lapse or evaluation of spindle formation), or flow cytometry.

Any of the parameters described herein may be assessed using standard techniques known to one of skill in the art, such as histology, microscopy, and various functional assays.

In some embodiments, the exogenous transcription unit encodes a therapeutic polypeptide (e.g., a protein), such as a clotting factor, growth factor, hormone, enzyme, cytokine (e.g., a pro-inflammatory cytokine or an anti-inflammatory cytokine), cytokine receptor, chimeric protein, fusion protein or lipoprotein. The polypeptide encoded by the exogenous transcription unit may have a naturally occurring amino acid sequence or may contain a variant of the naturally occurring sequence. The variant can be a non-naturally occurring or naturally occurring amino acid substitution, mutation, deletion or addition relative to the reference (e.g., naturally occurring) sequence. The naturally occurring amino acid sequence may be a polymorphic variant. The naturally occurring amino acid sequence can be a human or a non-human amino acid sequence. In some embodiments, the naturally occurring amino acid sequence is a human sequence. In some embodiments, the therapeutic polypeptide has about 2, 3, 4, 5, 6, 7, 8, 9, 10, 12, 14, 16, 18, 20, 25, 30, 35, 40, 45, or less than 50 amino acids. In some embodiments, the polypeptide has an average molecular weight of 5 kD, 10 kD, 25 kD, 50 kD, 100 kD, 150 kD, 200 kD, 250 kD, 500 kD, or more.

In some embodiments, the polypeptide is a hormone. Exemplary hormones include anti-diuretic hormone (ADH), oxytocin, growth hormone (GH), prolactin, growth hormone-releasing hormone (GHRH), thyroid stimulating hormone (TSH), thyrotropin-release hormone (TRH), adrenocorticotropic hormone (ACTH), follicle-stimulating hormone (FSH), luteinizing hormone (LH), luteinizing hormone-releasing hormone (LHRH), thyroxine, calcitonin, parathyroid hormone (PTH), aldosterone, cortisol, epinephrine, glucagon, insulin, estrogen, progesterone, and testosterone. In some embodiments, the polypeptide is insulin (e.g., insulin A-chain, insulin B-chain, or proinsulin). In some embodiments, the polypeptide is a growth hormone, such as human growth hormone (hGH), recombinant human growth hormone (rhGH), bovine growth hormone, methionine-human growth hormone, des-phenylalanine human growth hormone, and porcine growth hormone.

In some embodiments, the polypeptide is a growth factor, e.g., vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and -II (IGF-I and IGF-II).

In some embodiments, the polypeptide is a clotting factor or a coagulation factor, e.g., a blood clotting factor or a blood coagulation factor. In some embodiments, the polypeptide is involved in coagulation, i.e., the process by which blood is converted from a liquid to solid or gel. Exemplary clotting factors and coagulation factors include Factor I (e.g., fibrinogen), Factor II (e.g., prothrombin), Factor III (e.g., tissue factor), Factor V (e.g., proaccelerin, labile factor), Factor VI, Factor VII (e.g., stable factor, proconvertin), Factor VIII (e.g., antihemophilic factor A), Factor VIIIC, Factor IX (e.g., antihemophilic factor B), Factor X (e.g., Stuart-Prower factor), Factor XI (e.g., plasma thromboplastin antecedent), Factor XII (e.g., Hagerman factor), Factor XIII (e.g., fibrin-stabilizing factor), von Willebrand factor (vWF), prekallikrein, heparin cofactor II, high molecular weight kininogen (e.g., Fitzgerald factor), antithrombin III, and fibronectin. In some embodiments, the polypeptide is an anti-clotting factor, such as Protein C.

In some embodiments, the polypeptide is an immunoglobulin chain (heavy or light chain) or fragment thereof, comprising at least one immunoglobulin variable domain sequence, and optionally comprising an immunoglobulin Fc region. In an embodiment, the polypeptide a full-length immunoglobulin chain.

In some embodiments, the polypeptide is a cytokine or a cytokine receptor, or a chimeric protein including cytokines or their receptors, including, for example tumor necrosis factor alpha and beta, their receptors and their derivatives, renin, lipoproteins, colchicine; corticotrophin; vasopressin; somatostatin; lypressin; pancreozymin; leuprolide; alpha-1-antitrypsin; atrial natriuretic factor; lung surfactant; a plasminogen activator other than a tissue-type plasminogen activator (t-PA), for example a urokinase; bombesin; thrombin; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; chorionic gonadotropin; a microbial protein, such as beta-lactamase; DNase; inhibin; activin; receptors for hormones or growth factors; integrin; protein A or D; rheumatoid factors; platelet-derived growth factor (PDGF); epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; an interferon such as interferon-alpha (e.g., interferon.alpha.2A), -beta, -gamma, -lambda and consensus interferon; colony stimulating factors (CSFs), e.g., M-CSF, GM-CSF, and G-CSF; interleukins (ILs), e.g., IL-1, IL-2 to IL-10; superoxide dismutase; T-cell receptors; surface membrane proteins; decay accelerating factor; transport proteins; homing receptors; addressins; fertility inhibitors such as the prostaglandins; fertility promoters; regulatory proteins; antibodies (including fragments thereof) and chimeric proteins, such as immunoadhesins. Suitable polypeptides may be native or recombinant and include, e.g., fusion proteins.

Examples of a polypeptide that may be encoded by the exogenous transcription unit also include CCL1, CCL2 (MCP-1), CCL3 (MIP-1α), CCL4 (MIP-1β), CCL5 (RANTES), CCL6, CCL7, CCL8, CCL9 (CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1 (KC), CXCL2 (SDF1a), CXCL3, CXCL4, CXCL5, CXCL6, CXCL7, CXCL8 (IL8), CXCL9, CXCL10, CXCL11, CXCL12, CXCL13, CXCL14, CXCL15, CXCL16, CXCL17, CX3CL1, XCL1, XCL2, TNFA, TNFB (LTA), TNFC (LTB), TNFSF4, TNFSF5 (CD40LG), TNFSF6, TNFSF7, TNFSF8, TNFSF9, TNFSF10, TNFSF11, TNFSF13B, EDA, IL2, IL15, IL4, IL13, IL7, IL9, IL21, IL3, IL5, IL6, IL11, IL27, IL30, IL31, OSM, LIF, CNTF, CTF1, IL12a, IL12b, IL23, I127, IL35, IL14, IL16, I132, IL34, IL10, IL22, IL19, IL20, IL24, I126, I129, IFNL1, IFNL2, IFNL3, IL28, IFNA1, IFNA2, IFNA4, IFNA5, IFNA6, IFNA7, IFNA8, IFNA10, IFNA13, IFNA14, IFNA16, IFNA17, IFNA21, IFNB1, IFNK, IFNW1, IFNG, ILIA (IL1F1), IL1B (IL1F2), IL1Ra (IL1F3), IL1F5 (IL36RN), IL1F6 (IL36A), IL1F7 (IL37), IL1F8 (IL36B), IL1F9 (IL36G), ILIF10 (IL38), IL33 (ILIF11), IL18 (IL1G), IL17, KITLG, IL25 (IL17E), CSF1 (M-CSF), CSF2 (GM-CSF), CSF3 (G-CSF), SPP1, TGFB1, TGFB2, TGFB3, CCL3L1, CCL3L2, CCL3L3, CCL4L1, CCL4L2, IL17B, IL17C, IL17D, IL17F, AIMP1 (SCYE1), MIF, Areg, BC096441, Bmp1, Bmp10, Bmp15, Bmp2, Bmp3, Bmp4, Bmp5, Bmp6, Bmp7, Bmp8a, Bmp8b, C1qtnf4, Ccl21a, Ccl27a, Cd70, Cer1, Cklf, Clef1, Cmtm2a, Cmtm2b, Cmtm3, Cmtm4, Cmtm5, Cmtm6, Cmtm7, Cmtm8, Crlf1, Ctf2, Ebi3, Edn1, Fam3b, Fas1, Fgf2, Flt31, Gdf10, Gdf11, Gdf15, Gdf2, Gdf3, Gdf5, Gdf6, Gdf7, Gdf9, Gm12597, Gm13271, Gm13275, Gm13276, Gm13280, Gm13283, Gm2564, Gpil, Grem1, Grem2, Grn, Hmgb1, Ifna11, Ifna12, Ifna9, Ifnab, Ifne, Il17a, Il23a, II25, II31, Iltifb, Inhba, Lefty1, Lefty2, Mstn, Nampt, Ndp, Nodal, Pf4, Pglyrp1, Prl7d1, Scg2, Scgb3a1, Slurp1, Spp1, Thpo, Tnfsf10, Tnfsf11, Tnfsf12, Tnfsf13, Tnfsf13b, Tnfsf14, Tnfsf15, Tnfsf18, Tnfsf4, Tnfsf8, Tnfsf9, Tslp, Vegfa, Wnt1, Wnt2, Wnt5a, Wnt7a, Xcl1, epinephrine, melatonin, triiodothyronine, a prostaglandin, a leukotriene, prostacyclin, thromboxane, islet amyloid polypeptide, müllerian inhibiting factor or hormone, adiponectin, corticotropin, angiotensin, vasopressin, arginine vasopressin, atriopeptin, brain natriuretic peptide, calcitonin, cholecystokinin, cortistatin, enkephalin, endothelin, erythropoietin, follicle-stimulating hormone, galanin, gastric inhibitory polypeptide, gastrin, ghrelin, glucagon, glucagon-like peptide-1, gonadotropin-releasing hormone, hepcidin, human chorionic gonadotropin, human placental lactogen, inhibin, somatomedin, leptin, lipotropin, melanocyte stimulating hormone, motilin, orexin, oxytocin, pancreatic polypeptide, pituitary adenylate cyclase-activating peptide, relaxin, renin, secretin, somatostatin, thrombopoietin, thyrotropin, thyrotropin-releasing hormone, vasoactive intestinal peptide, androgen, alpha-glucosidase (also known as acid maltase), glycogen phosphorylase, glycogen debrancher enzyme, phosphofructokinase, phosphoglycerate kinase, phosphoglycerate mutase, lactate dehydrogenase, carnitine palymityl transferase, carnitine, and myoadenylate deaminase.

In some embodiments, the polypeptide is a replacement therapy or a replacement protein.

In some embodiments, the replacement therapy or replacement protein is a clotting factor or a coagulation factor, e.g., Factor VII, Factor VIII or Factor IX.

In some embodiments, the replacement therapy or replacement protein is an enzyme, e.g., alpha-galactosidase A (GLA), alpha-L-iduronidase (IDUA), glucocerebrosidase, or N-sulfoglucosamine sulfohydrolase (SGSH). In an embodiment, the engineered mammalian cell comprises an exogenous nucleic acid encoding the IDUA.

In an embodiment, the engineered mammalian cells are not islet cells, as defined herein. In an embodiment, the engineered mammalian cells have one or more of the following characteristics: (i) are not capable of producing insulin (e.g., insulin A-chain, insulin B-chain, or proinsulin) in an amount effective to treat diabetes or another disease or condition that may be treated with insulin; (ii) not capable of producing insulin in a glucose-responsive manner; or (iii) not derived from an induced pluripotent stem cell that was engineered or differentiated into insulin-producing pancreatic beta cells.

### Features of Implantable Elements

An engineered mammalian cell described herein or a plurality of such cells may be incorporated into an implantable element for use in treating a disease or disorder in a subject, as well as for reducing the level of pericapsular fibrotic overgrowth on the implantable element upon implantation in a subject.

An implantable element of the present disclosure comprises at least one barrier that prevents immune cells from contacting cells contained inside the device. At least a portion of the barrier needs to be sufficiently porous to allow a therapeutic agent expressed and secreted by the cells to exit the device. A variety of device configurations known in the art are suitable.

The device (e.g., particle) can have any configuration and shape appropriate for supporting the viability and productivity of the contained cells after implant into the intended target location. As non-limiting examples, device shapes may be cylinders, rectangles, disks, ovoids, stellates, or spherical. The device can be comprised of a mesh-like or nested structure. In some embodiments, a device is capable of preventing materials over a certain size from passing through a pore or opening. In some embodiments, a device (e.g., particle) is capable of preventing materials greater than 50 kD, 75 kD, 100 kD, 125 kD, 150 kD, 175 kD, 200 kD, 250 kD, 300 kD, 400 kD, 500 kD, 750 kD, or 1,000 kD from passing through.

In an embodiment, the device is a macroencapsulation device. Nonlimiting examples of macrodevices are described in: WO 2019/068059, WO 2019/169089, U.S. Pat. Nos. 9,526,880, 9,724,430 and 8,278,106; European Patent No. EP742818B1, and Sang, S. and Roy, S., Biotechnol. Bioeng. 113(7):1381-1402 (2016).

In an embodiment, the device is a macrodevice having one or more cell-containing compartments. A device with two or more cell-containing compartments may be configured to produce two or more proteins, e.g., cells expressing a first therapeutic agent would be placed in one compartment and cells expressing a different protein (e.g., a therapeutic protein) would be placed in a separate compartment. WO 2018/232027 describes a device with multiple cell-containing compartments formed in a micro-fabricated body and covered by a porous membrane.

In an embodiment, the device is configured as a thin, flexible strand as described in U.S. Pat. No. 10,493,107. This strand comprises a substrate, an inner polymeric coating surrounding the substrate and an outer hydrogel coating surrounding the inner polymeric coating. The protein-expressing cells are positioned in the outer coating.

In some embodiments, a device (e.g., particle) has a largest linear dimension (LLD), e.g., mean diameter, or size that is at least about 0.5 millimeter (mm), preferably about 1.0 mm, about 1.5 mm or greater. In some embodiments, a device can be as large as 10 mm in diameter or size.

For example, a device or particle described herein is in a size range of 0.5 mm to 10 mm, 1 mm to 10 mm, 1 mm to 8 mm, 1 mm to 6 mm, 1 mm to 5 mm, 1 mm to 4 mm, 1 mm to 3 mm, 1 mm to 2 mm, 1 mm to 1.5 mm, 1.5 mm to 8 mm, 1.5 mm to 6 mm, 1.5 mm to 5 mm, 1.5 mm to 4 mm, 1.5 mm to 3 mm, 1.5 mm to 2 mm, 2 mm to 8 mm, 2 mm to 7 mm, 2 mm to 6 mm, 2 mm to 5 mm, 2 mm to 4 mm, 2 mm to 3 mm, 2.5 mm to 8 mm, 2.5 mm to 7 mm, 2.5 mm to 6 mm, 2.5 mm to 5 mm, 2.5 mm to 4 mm, 2.5 mm to 3 mm, 3 mm to 8 mm, 3 mm to 7 mm, 3 mm to 6 mm, 3 mm to 5 mm, 3 mm to 4 mm, 3.5 mm to 8 mm, 3.5 mm to 7 mm, 3.5 mm to 6 mm, 3.5 mm to 5 mm, 3.5 mm to 4 mm, 4 mm to 8 mm, 4 mm to 7 mm, 4 mm to 6 mm, 4 mm to 5 mm, 4.5 mm to 8 mm, 4.5 mm to 7 mm, 4.5 mm to 6 mm, 4.5 mm to 5 mm, 5 mm to 8 mm, 5 mm to 7 mm, 5 mm to 6 mm, 5.5 mm to 8 mm, 5.5 mm to 7 mm, 5.5 mm to 6 mm, 6 mm to 8 mm, 6 mm to 7 mm, 6.5 mm to 8 mm, 6.5 mm to 7 mm, 7 mm to 8 mm, or 7.5 mm to 8 mm.

In some embodiments, a device of the disclosure (e.g., particle, capsule) comprises at least one pore or opening, e.g., to allow for the free flow of materials. In some embodiments, the mean pore size of a device is between about 0.1 μm to about 10 μm. For example, the mean pore size may be between 0.1 μm to 10 m, 0.1 μm to 5 m, 0.1 m to 2 μm, 0.15 μm to 10 m, 0.15 μm to 5 μm, 0.15 μm to 2 μm, 0.2 μm to 10 μm, 0.2 μm to 5 μm, 0.25 μm to 10 μm, 0.25 μm to μm, 0.5 μm to 10 μm, 0.75 m to 10 μm, 1 m to 10 μm, 1 m to 5 μm, 1 m to 2 μm, 2 m to 10 m, 2 μm to 5 m, or 5 m to 10 μm. In some embodiments, the mean pore size of a device is between about 0.1 μm to 10 μm. In some embodiments, the mean pore size of a device is between about 0.1 μm to 5 m. In some embodiments, the mean pore size of a device is between about 0.1 μm to 1 μm.

In some embodiments, the device comprises a semi-permeable, biocompatible membrane surrounding the genetically modified cells that are encapsulated in a polymer composition (e.g., an alginate hydrogel). The membrane pore size is selected to allow oxygen and other molecules important to cell survival and function to move through the semi-permeable membrane while preventing immune cells from traversing through the pores. In an embodiment, the semi-permeable membrane has a molecular weight cutoff of less than 1000 kD or between 50-700 kD, 70-300 kD, or between 70-150 kD, or between 70 and 130 kD.

In an embodiment, the device may contain a cell-containing compartment that is surrounded with a barrier compartment formed from a cell-free biocompatible material, such as the core-shell microcapsules described in Ma, M et al., Adv. Healthc Mater., 2(5):667-672 (2012). Such a barrier compartment could be used with or without the semi-permeable membrane.

Cells in the cell-containing compartment(s) of a device of the disclosure may be encapsulated in a polymer composition. The polymer composition may comprise one or more hydrogel-forming polymers. In addition to the polymer composition in the cell-containing compartment(s), the device (e.g., macrodevice, particle, hydrogel capsule) may comprise or be formed from materials such as metals, metallic alloys, ceramics, polymers, fibers, inert materials, and combinations thereof. A device may be completely made up of one type of material, or may comprise other materials within the cell-containing compartment and any other compartments.

In some embodiments, the device comprises a metal or a metallic alloy. In an embodiment, one or more of the compartments in the device (e.g., the first compartment, the second compartment, or all compartments) comprises a metal or a metallic alloy. Exemplary metallic or metallic alloys include comprising titanium and titanium group alloys (e.g., nitinol, nickel titanium alloys, thermo-memory alloy materials), platinum, platinum group alloys, stainless steel, tantalum, palladium, zirconium, niobium, molybdenum, nickel-chrome, chromium molybdenum alloys, or certain cobalt alloys (e.g., cobalt-chromium and cobalt-chromium-nickel alloys, e.g., ELGILOY® and PHYNOX®). For example, a metallic material may be stainless steel grade 316 (SS 316L) (comprised of Fe, <0.3% C, 16-18.5% Cr, 10-14% Ni, 2-3% Mo, <2% Mn, <1% Si, <0.45% P, and <0.03% S). In metal-containing devices, the amount of metal (e.g., by % weight, actual weight) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, the device comprises a ceramic. In an embodiment, one or more of the compartments in the device (e.g., the first compartment, the second compartment, or all compartments) comprises a ceramic. Exemplary ceramic materials include oxides, carbides, or nitrides of the transition elements, such as titanium oxides, hafnium oxides, iridium oxides, chromium oxides, aluminum oxides, and zirconium oxides. Silicon based materials, such as silica, may also be used. In ceramic-containing devices, the amount of ceramic (e.g., by % weight, actual weight) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, the device has two hydrogel compartments, in which the inner, cell-containing compartment is completely surrounded by the second, outer (e.g., barrier) compartment. In an embodiment, the inner boundary of the second compartment forms an interface with the outer boundary of the first compartment. In such embodiments, the thickness of the second (outer) compartment means the average distance between the outer boundary of the second compartment and the interface between the two compartments, e.g., the average of the distances measured at each of the thinnest and thickest points visually observed in the outer compartment. In some embodiments (e.g., the device is about 1.5 mm in diameter), the thinnest and thickest distances for the outer compartment are between 25 and 110 micrometers (μm) and between 270 and 480 m, respectively. In some embodiments, the thickness of the outer compartment is greater than about 10 nanometers (nm), preferably 100 nm or greater and can be as large as 1 millimeter (mm). For example, the thickness (e.g., average distance) of the outer compartment in a hydrogel capsule device described herein may be 10 nm to 1 mm, 100 nm to 1 mm, 500 nm to 1 millimeter, 1 micrometer (μm) to 1 mm, 1 μm to 1 mm, 1 μm to 500 μm, 1 μm to 250 μm, 1 μm to 1 mm, 5 m to 500 m, 5 μm to 250 μm, 10 μm to 1 mm, 10 m to 500 μm, or 10 μm to 250 μm. In some embodiments, the thickness (e.g., average distance) of the outer compartment is 100 nm to 1 mm, between 1 μm and 1 mm, between 1 μm and 500 μm or between 5 μm and 1 mm. In some embodiments, the thickness (e.g., average distance) of the outer compartment is between about 50 μm and about 100 μm. In some embodiments (e.g., the device is about 1.5 mm in diameter), the thickness of the outer compartment (e.g., average distance) is between about 180 m and 260 m or between about 310 μm and 440 m.

In some embodiments of a two-compartment hydrogel capsule device, the mean pore size of the cell-containing inner compartment and the outer compartment is substantially the same. In some embodiments, the mean pore size of the inner compartment and the second compartment differ by about 1.5%, 2%, 5%, 7.5%, 10, 1%, 20%, 25%, 30%, 35%, 40%, 45%, 50%, 55%, 60%, 65%, 70%, 75%, or more. In some embodiments, the mean pore size of the device (e.g., mean pore size of the first compartment and/or mean pore size of the second compartment) is dependent on a number of factors, such as the material(s) within each compartment and the presence and density of a compound of Formula (I).

In some embodiments, the polymer composition in the cell-containing compartment(s) comprises a polysaccharide or other hydrogel-forming polymer (e.g., alginate, hyaluronate or chondroitin). In some embodiments, the polymer is an alginate, which is a polysaccharide made up of β-D-mannuronic acid (M) and α-L-guluronic acid (G). In some embodiments, the alginate has a low molecular weight (e.g., approximate molecular weight of <75 kD) and G:M ratio≥1.5, (ii) a medium molecular weight alginate, e.g., has approximate molecular weight of 75-150 kDa and G:M ratio ≥1.5, (iii) a high molecular weigh alginate, e g., has an approximate MW of 150 kDa-250 kDa and G:M ratio≥1.5, (iv) or a blend of two or more of these alginates. In some embodiments, the cell-containing compartment(s) further comprises at least one cell-binding substance (CBS), e.g., a cell-binding peptide (CBP) or cell-binding polypeptide (CBPP) described in WO2020069429.

In some embodiments, the cell-containing compartment(s) comprises an alginate covalently modified with a linker-cell-binding peptide moiety, e.g., GRGD or GRGDSP. In an embodiment, the cell-binding peptide density in the cell-containing compartment(s) (% nitrogen as determined by combustion analysis, e.g., as described in WO2020198695) to be at least 0.05%, 0.1%, 0.2% or 0.3% but less than 4%, 3%, 2% or 1%. In an embodiment, the total density of the linker-CBP in a cell containing compartment is about 0.1 to about 1.0 micromoles of the CBP per g of CBP-polymer (e.g., a MM4W-alginate covalently modified with GRGD or GRGDSP in solution as determined by a quantitative peptide conjugation assay, e.g., an assay described in WO2020198695. In an embodiment, the linker-CBP is GRGDSP and the alginate has a molecular weight of 75 kDa to 150 kDa and a G:M ratio of greater than or equal to 1.5. In an embodiment, the cell-containing compartment also comprises an unmodified alginate with a molecular weight of 75 kDa to 150 kDa and a G:M ratio of greater than or equal to 1.5.

The device may form part of a plurality of substantially the same devices in a preparation (e.g., composition). In some embodiments, the devices (e.g., particles, hydrogel capsules) in the preparation have a mean diameter or size between about 0.5 mm to about 8 mm. In some embodiments, the mean diameter or size of devices in the preparation is between about 0.5 mm to about 4 mm or between about 0.5 mm to about 2 mm. In some embodiments, the devices in the preparation are two-compartment hydrogel capsules and have a mean diameter or size of about 0.7 mm to about 1.3 mm or about 1.2 mm to about 1.8 mm.

In some embodiments, the surface of the device comprises a compound capable of mitigating the FBR upon implant into a subject, an afibrotic compound as described herein below. For devices comprising a barrier compartment surrounding the cell-containing compartment, the afibrotic compound may covalently modify a polymer disposed throughout the barrier compartment and optionally throughout the cell-containing compartment.

In some embodiments, one or more compartments in a device comprises an afibrotic polymer, e.g., an afibrotic compound of Formula (I) covalently attached to a polymer. In an embodiment, some or all the monomers in the afibrotic polymer are modified with the same compound of Formula (I). In some embodiments, some or all the monomers in the afibrotic polymer are modified with different compounds of Formula (I). In some embodiments in which the device is a 2-compartment hydrogel capsule, the afibrotic polymer is present only in the outer, barrier compartment.

One or more compartments in a device may comprise an unmodified polymer that is the same or different than the polymer in any afibrotic polymer that is present in the device. In an embodiment, the first compartment, second compartment or all compartments in the device comprise the unmodified polymer.

Each of the modified and unmodified polymers in the device may be a linear, branched, or cross-linked polymer, or a polymer of selected molecular weight ranges, degree of polymerization, viscosity or melt flow rate. Branched polymers can include one or more of the following types: star polymers, comb polymers, brush polymers, dendronized polymers, ladders, and dendrimers. A polymer may be a thermoresponsive polymer, e.g., gel (e.g., becomes a solid or liquid upon exposure to heat or a certain temperature) or a photocrosslinkable polymer. Exemplary polymers include polystyrene, polyethylene, polypropylene, polyacetylene, poly(vinyl chloride) (PVC), polyolefin copolymers, poly(urethane)s, polyacrylates and polymethacrylates, polyacrylamides and polymethacrylamides, poly(methyl methacrylate), poly(2-hydroxyethyl methacrylate), polyesters, polysiloxanes, polydimethylsiloxane (PDMS), polyethers, poly(orthoester), poly(carbonates), poly(hydroxyalkanoate)s, polyfluorocarbons, PEEK®, Teflon® (polytetrafluoroethylene, PTFE), PEEK, silicones, epoxy resins, Kevlar®, Dacron® (a condensation polymer obtained from ethylene glycol and terephthalic acid), polyethylene glycol, nylon, polyalkenes, phenolic resins, natural and synthetic elastomers, adhesives and sealants, polyolefins, polysulfones, polyacrylonitrile, biopolymers such as polysaccharides and natural latex, collagen, cellulosic polymers (e.g., alkyl celluloses, etc.), polyethylene glycol and 2-hydroxyethyl methacrylate (HEMA), polysaccharides, poly(glycolic acid), poly(L-lactic acid) (PLLA), poly(lactic glycolic acid) (PLGA), a polydioxanone (PDA), or racemic poly(lactic acid), polycarbonates, (e.g., polyamides (e.g., nylon)), fluoroplastics, carbon fiber, agarose, alginate, chitosan, and blends or copolymers thereof. In polymer-containing devices, the amount of a polymer (e.g., by % weight of the device, actual weight of the polymer) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, one or more of the modified and unmodified polymers in the device comprises a polyethylene. Exemplary polyethylenes include ultra-low-density polyethylene (ULDPE) (e.g., with polymers with densities ranging from 0.890 to 0.905 g/cm³, containing comonomer); very-low-density polyethylene (VLDPE) (e.g., with polymers with densities ranging from 0.905 to 0.915 g/cm³, containing comonomer); linear low-density polyethylene (LLDPE) (e.g., with polymers with densities ranging from 0.915 to 0.935 g/cm³, contains comonomer); low-density polyethylene (LDPE) (e.g., with polymers with densities ranging from about 0.915 to 0.935 g/m³); medium density polyethylene (MDPE) (e.g., with polymers with densities ranging from 0.926 to 0.940 g/cm³, may or may not contain comonomer); high-density polyethylene (HDPE) (e.g., with polymers with densities ranging from 0.940 to 0.970 g/cm³, may or may not contain comonomer) and polyethylene glycol.

In some embodiments, one or more of the modified and unmodified polymers in the device comprises a polypropylene. Exemplary polypropylenes include homopolymers, random copolymers (homophasic copolymers), and impact copolymers (heterophasic copolymers), e.g., as described in McKeen, Handbook of Polymer Applications in Medicine and Medical Devices, 3—Plastics Used in Medical Devices, (2014):21-53.

In some embodiments, one or more of the modified and unmodified polymers in the device comprises a polypropylene. Exemplary polystyrenes include general purpose or crystal (PS or GPPS), high impact (HIPS), and syndiotactic (SPS) polystyrene.

In some embodiments, one or more of the modified and unmodified polymers comprises a comprises a thermoplastic elastomer (TPE). Exemplary TPEs include (i) TPA—polyamide TPE, comprising a block copolymer of alternating hard and soft segments with amide chemical linkages in the hard blocks and ether and/or ester linkages in the soft blocks; (ii) TPC—co-polyester TPE, consisting of a block copolymer of alternating hard segments and soft segments, the chemical linkages in the main chain being ester and/or ether; (iii) TPO—olefinic TPE, consisting of a blend of a polyolefin and a conventional rubber, the rubber phase in the blend having little or no cross-linking; (iv) TPS—styrenic TPE, consisting of at least a triblock copolymer of styrene and a specific diene, where the two end blocks (hard blocks) are polystyrene and the internal block (soft block or blocks) is a polydiene or hydrogenated polydiene; (v) TPU—urethane TPE, consisting of a block copolymer of alternating hard and soft segments with urethane chemical linkages in the hard blocks and ether, ester or carbonate linkages or mixtures of them in the soft blocks; (vi) TPV—thermoplastic rubber vulcanizate consisting of a blend of a thermoplastic material and a conventional rubber in which the rubber has been cross-linked by the process of dynamic vulcanization during the blending and mixing step; and (vii) TPZ—unclassified TPE comprising any composition or structure other than those grouped in TPA, TPC, TPO, TPS, TPU, and TPV.

In some embodiments, the unmodified polymer is an unmodified alginate. In some embodiments, the alginate is a high guluronic acid (G) alginate, and comprises greater than about 50%, 55%, 60%, 65%, 70%, 75%, 80%, 85%, 90%, or more guluronic acid (G). In some embodiments, the alginate is a high mannuronic acid (M) alginate, and comprises greater than about 50%, 55%, 60%, 65%, 70%, 75%, 80%, 85%, 90%, or more mannuronic acid (M). In some embodiments, the ratio of M:G is about 1. In some embodiments, the ratio of M:G is less than 1. In some embodiments, the ratio of M:G is greater than 1. In an embodiment, the unmodified alginate has a molecular weight of 150 kDa-250 kDa and a G:M ratio of ≥1.5.

In some embodiments, the afibrotic polymer comprises an alginate chemically modified with a Compound of Formula (I). The alginate in the afibrotic polymer may be the same or different than any unmodified alginate that is present in the device. In an embodiment, the density of the Compound of Formula (I) in the afibrotic alginate (e.g., amount of conjugation) is between about 4.0% and about 8.0%, between about 5.0% and about 7.0%, or between about 6.0% and about 7.0% nitrogen (e.g., as determined by combustion analysis for percent nitrogen). In an embodiment, the amount of Compound 101 produces an increase in % N (as compared with the unmodified alginate) of about 0.5% to 2% 2% to 4% N, about 4% to 6% N, about 6% to 8%, or about 8% to 10% N), where % N is determined by combustion analysis and corresponds to the amount of Compound 101 in the modified alginate.

In other embodiments, the density (e.g., concentration) of the Compound of Formula (I) (e.g., Compound 101) in the afibrotic alginate is defined as the % w/w, e.g., % of weight of amine/weight of afibrotic alginate in solution (e.g., saline) as determined by a suitable quantitative amine conjugation assay (e.g. by an assay described in WO2020069429), and in certain embodiments, the density of a Compound of Formula (I) (e.g., Compound 101) is between about 1.0% w/w and about 3.0% w/w, between about 1.3% w/w and about 2.5% w/w or between about 1.5% w/w and 2.2% w/w.

In alginate-containing devices, the amount of modified and unmodified alginates (e.g., by % weight of the device, actual weight of the alginate) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

The alginate in an afibrotic polymer can be chemically modified with a compound of Formula (I) using any suitable method known in the art. For example, the alginate carboxylic acid moiety can be activated for coupling to one or more amine-functionalized compounds to achieve an alginate modified with a compound of Formula (I). The alginate polymer may be dissolved in water (30 mL/gram polymer) and treated with 2-chloro-4,6-dimethoxy-1,3,5-triazine (0.5 eq) and N-methylmorpholine (1 eq). To this mixture may be added a solution of the compound of Formula (I) in acetonitrile (0.3M). The reaction may be warmed to 55 °C for 16 h, then cooled to room temperature and gently concentrated via rotary evaporation, then the residue may be dissolved, e.g., in water. The mixture may then be filtered, e.g., through a bed of cyano-modified silica gel (Silicycle) and the filter cake washed with water. The resulting solution may then be dialyzed (10,000 MWCO membrane) against water for 24 hours, e.g., replacing the water twice. The resulting solution can be concentrated, e.g., via lyophilization, to afford the desired chemically modified alginate.

In an embodiment, modified polymers described herein may be covalently bound to a photoactive crosslinker. A photoactive crosslinkers is a moiety that is activated upon exposure to light. The light may comprise any wavelength of light, from infrared to x-ray energy. In some embodiments, the light comprises ultraviolet light (e.g., between 360 nm to 400 nm, e.g., 370 nm to 390 nm, e.g., 380 nm to 400 nm, e.g., 390 nm to 400 nm). In some embodiments, the light comprises visible light (e.g., between 400 nm to 700 nm). Photoactive crosslinkers often include at least one unsaturated functional group capable of undergoing free radical polymerization. In an embodiment, a photoactive crosslinker comprises an alkenyl group (e.g, C2-C12 alkenyl, C2-C8 alkenyl). In an embodiment, a photoactive crosslinker comprises an alkynyl group (e.g, C2-C12 alkynyl, C2-C8 alkynyl). Moieties that may be activated upon exposure to irradiation include aromatic groups, alkenyl groups, alkynyl groups, and azide groups. Exemplary alkenyl compounds that may act as photoactive crosslinkers include alkenoic acids such as acrylate, methacrylate, acrylamide, and methacrylamide and their corresponding acid chlorides and anhydrides. Other exemplary alkenyl compounds include enols (e.g., 2-propen-1-ol), alkenyl halides (such as allyl chloride, and the like), organometallic alkenyl compounds (such as vinyl magnesium bromide), aryl compounds (e.g., styrene). Exemplary photoactive crosslinkers include acrylate, methacrylate, ethylene glycol dimethylacrylate, divinylbenzene, 1,3-diisopropyl benzene, and N,N′-methylenebisacrylamide. In an embodiment, the photoactive crosslinker is a bifunctional crosslinker, i.e., has two reactive functional groups. In an embodiment, the photoactive covalent crosslinker has both alkenyl and amide functional groups. In an embodiment, the photoactive crosslinker has both alkenyl and carboxylate functional groups. In an embodiment, the photoactive crosslinker has both alkenyl and amide functional groups.

In an embodiment, the modified polymers described herein comprise a photoactive crosslinker having the structure of Formula (IV).

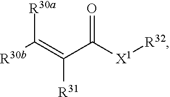

or a pharmaceutically acceptable salt or tautomer thereof, wherein X¹ is absent, O, NR³³, or C(R^{34a})(R^{34b}); each of R^{30a}, R^{30b}, R³¹, R³², R³³, R^{34a}, and R^{34b} is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In an embodiment, X¹ is O, each of R^{30a}, R^{30b}, R³¹, and R³² is hydrogen, and R³² is heteroalkyl (e.g., propylamine, e.g., —CH₂CH₂CH₂NH₂). In an embodiment, X¹ is O, each of R^{30a}, R^{30b}, R³¹, and R³² is hydrogen, and R³² is heteroalkyl (e.g., ethylamine, e.g., —CH₂CH₂NH₂). In an embodiment, the photoactive crosslinker of Formula (IV) is methacrylate. In an embodiment X¹ is absent; R³² is halo (e.g., chloro); and each of R^{30a}, R^{30b} and R³¹ is hydrogen. In an embodiment, the photoactive crosslinker of Formula (IV) is acryloyl chloride.

In an embodiment, X¹ is NR³³ (e.g., NH), and each of R^{30a}, R^{30b}, R³¹, and R³² is hydrogen. In an embodiment, the photoactive crosslinker of Formula (IV) is acrylamide.

In an embodiment, the modified polymers described herein comprise a photoactive crosslinker having the structure of Formula (IV-a):

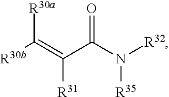

or a pharmaceutically acceptable salt or tautomer thereof, wherein each of R^{30a}, R^{30b}, R³¹, R³² and R³⁵ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In an embodiment, the modified polymers described herein comprise a photoactive crosslinker having the structure of Formula (IV-b):

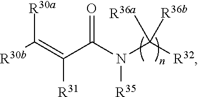

or a pharmaceutically acceptable salt or tautomer thereof, wherein each of R^{30a}, R^{3b}, R³¹, R³², R^{36a}, and R^{36b} is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; R³⁵ is hydrogen, alkyl, heteroalkyl, halo, cyano, nitro, amino, cycloalkyl, heterocyclyl, aryl, or heteroaryl; and n is 1, 2, 3, 4, 5, or 6.

In an embodiment, the modified polymers described herein comprise a photoactive crosslinker having the structure of Formula (IV-c):

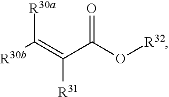

or a pharmaceutically acceptable salt or tautomer thereof, wherein each of R^{30a}, R^{30b}, and R³¹ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; R³² is alkyl, alkenyl, alkynyl, heteroalkyl,—C(O)OR^{A1}, —C(O)R^{B1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In an embodiment, the modified polymers described herein comprise a photoactive crosslinker having the structure of Formula (IV-d):

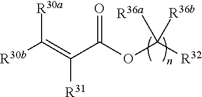

or a pharmaceutically acceptable salt or tautomer thereof, wherein each of R^{30a}, R^{30b}, R³¹, R³², R^{36a}, and R^{36b} is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; R³² is alkyl, alkenyl, alkynyl, heteroalkyl, —C(O)OR^{A1}, —C(O)R^{B1}, cycloalkyl, heterocyclyl, aryl, or heteroaryl; each R^{A1}, R^{B1}, R^{C1}, R^{D11}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; and n is 1, 2, 3, 4, 5, or 6.

Photoactive crosslinkers may be used alone or, preferably in the presence of a photoinitiator. A “photoinitiator,” as used herein, refers to a molecule capable of absorbing radiation e.g., light e.g., photons, and forming a reactive species in an excited state. A variety of free radical initiators, as can readily be identified by those of skill in the art, can be employed in the practice of the present invention. In an embodiment, a photoinitiator is an ultraviolent (UV) photoinitiator. Exemplary UV photoinitiators include lithium phenyl-2,4,i-trimethylbenzoylphosphinate (LAP), camphorquinone, benzoin menthyl ether, 1-hydroxy-cyclohexyl-phenyl-ketone (i e., Irgacure 184), 2-hydroxy-2-methyl-1-phenyl-1-propanone (i.e. Darocur 1173) 2-hydroxy-1-[4-(2-hydroxyethoxy)phenyl]-2-methylpropan-1-one (i.e., Irgacure 2959), 2-benzyl-2-(dimethyamino)-1-(4-morpholin-4-ylphenyl)butan-1-one (i.e., Irgacure 369), 2-methyl-1-(4-methylsulfanylphenyl)2-morpholin-4-ylpropan-1-one (i.e., Irgacure 907) diphenyl(2,4,6-trimethylbenzoyl)phosphine oxide (e.g., Darocur TPO), benzoin ethyl ether, benzophenone, 9,10-anthraquinone, ethyl-4-N,N-dimethylaminobenzoate, diphenyliodonium chloride, and water soluble derivatives thereof. For visible light polymerization, a system of dye and cocatalyst may be used. Exemplary visible light photoinitiators include 2-(2,4,5,7-tetrabromo-3-hydroxy-6-oxoxanthen-9-yl)benzoic acid (i.e., Eosin Y), erythrosine, riboflavin, rose Bengal, methylene blue, and thionine. A small amount of a comonomer can optionally be added to the crosslinking reaction to increase the polymerization rates. Examples of suitable comonomers include vinyl pyrrolidinone, acrylamide, methacrylamide, acrylic acid, methacrylic acid, sodium acrylate, sodium methacrylate, hydroxyethyl acrylate, hydroxyethyl methacrylate (HEMA), ethylene glycol diacrylate, ethylene glycol dimethacrylate, pentaerythritol triacrylate, pentaerythritol trimethacrylate, trimethylol propane triacrylate, trimethylol propane trimethacrylate, tripropylene glycol diacrylate, tripropylene glycol dimethacrylate, glyceryl acrylate, glyceryl methacrylate, and the like. In some embodiments, the photoinitiator is a thermally activated photoinitiator.

A photoactive crosslinker may be used in the presence of a single photoinitiator or a plurality of photoinitiators. The plurality of photoinitiators may include 2, 3, 4, 5, 6, 7, 8, or more photoinitiators. In an embodiment, the covalent crosslinking moiety is present on the polysaccharide polymer at a density of at least 1%, e.g., 2%, 3%, 4%, 5%, 6%, 7%, 8%, 9%, 10%, or more, e.g., as determined by LC-UV assay.

The photoactive crosslinker may be covalently bound to a polysaccharide, e.g., an alginate. The modified polysaccharide polymer, e.g., modified alginate polymer, may be capable of being crosslinked to another polymer. In an embodiment, the polysaccharide polymer is modified with more than one type of photoactive crosslinker.

In an embodiment, the modified polysaccharide is a compound of Formula (V):

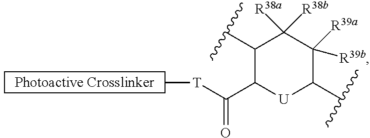

or a pharmaceutically acceptable salt or tautomer thereof, wherein each of T and U is independently C(R⁴⁰)(R⁴¹), O, or N(R⁴²); each of R^{30a}, R^{38b}, R^{39a}, R^{39b}, R⁴⁰, R⁴¹,and R⁴² is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR_{E1}, cycloalkyl, heterocyclyl, aryl, or heteroaryl; each of R³² and R³⁵ is hydrogen, alkyl, heteroalkyl, halo, cyano, nitro, amino, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; and the photoactive crosslinker has the structure of Formula (IV), (IV-a), (IV-b), (IV-c), or (IV-d).

In an embodiment, the photoactive crosslinker of Formula (V) has the structure of Formula (V-a):

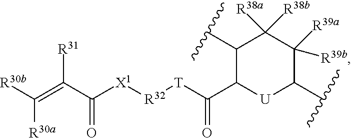

or a pharmaceutically acceptable salt or tautomer thereof, wherein each of T and U is independently C(R⁴⁰)(R⁴¹), O, or N(R⁴²); each of R^{30a}, R^{30b}, R³¹, R³², R^{38a}, R^{38b}, R^{39a}, R^{39b}, R⁴⁰, R⁴¹, and R⁴² is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In an embodiment, the modified polymer described herein has the structure of Formula (V-b):

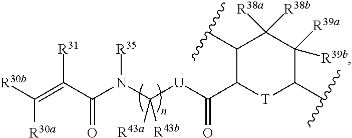

or a pharmaceutically acceptable salt or tautomer thereof, wherein each of U and T is independently C(R⁴⁰)(R⁴¹), O, or N(R⁴²); each of R^{30a}, R^{30b}, R³¹, R³⁵, R^{38a}, R^{38b}, R^{39a}, R^{39b}, R⁴⁰, R⁴¹, R⁴², R^{43a}, and R^{43b} is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; and n is 1, 2, 3, 4, 5, or 6.

In an embodiment, the modified polymer described herein has the structure of Formula (V-c):

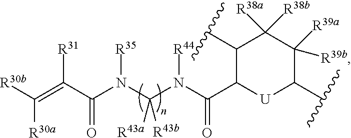

or a pharmaceutically acceptable salt or tautomer thereof, wherein U is C(R⁴⁰)(R⁴¹), O, or N(R⁴²); each of R^{30a}, R^{30b}, R³¹, R³⁵, R^{38a}, R^{38b}, R^{39a}, R^{39b}, R⁴⁰, R⁴¹, R⁴², R^{43a}, R^{43b} and R⁴⁴ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl; and n is 1, 2, 3, 4, 5, or 6.

In an embodiment, the modified polymer described herein has the structure of Formula (V-d):

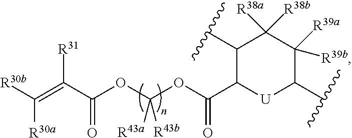

or a pharmaceutically acceptable salt or tautomer thereof, wherein U is C(R⁴⁰)(R⁴¹), O, or N(R⁴²); each of R^{30a}, R^{30b}, R³¹, R^{38a}, R^{38b}, R^{39a}, R^{39b}, R⁴⁰, R⁴¹, R⁴², R^{43a}, and R^{43b} is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; and n is 1, 2, 3, 4, 5, or 6.

In an embodiment, the modified polymer described herein comprises the structure of Formula (VI):

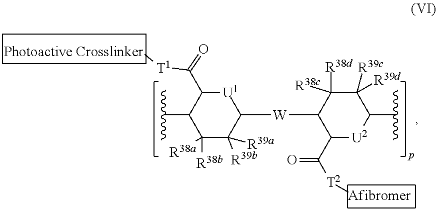

or a pharmaceutically acceptable salt or tautomer thereof, wherein each of W, T¹, T², U¹, and U² is independently C(R⁴⁰)(R⁴¹), O, or N(R⁴²); each of R^{38a}, R^{38b}, R^{38c}, R^{38d} R^{39a}, R^{39b}, R³⁹, R^{39b}, R⁴, R⁴¹, and R⁴² is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl; p is an integer from 1-100; the afibromer has the structure of Formula (I) or a subgenera of Formula (I) as described herein.

In an embodiment, the device comprises at least one cell-containing compartment, and in some embodiments contains two, three, four or more cell-containing compartments. In an embodiment, each cell-containing compartment comprises a plurality of cells (e.g., live cells) and the cells in at least one of the compartments are capable of expressing and secreting a mammalian protein when the device is implanted into a subject.

In an embodiment, all the cells in a cell-containing compartment are derived from a single parental cell-type or a mixture of at least two different parental cell types. In an embodiment, all of the cells in a cell-containing compartment are derived from the same parental cell type. In devices with two or more cell-containing compartments, the cells and the protein(s) produced thereby may be the same or different in each cell-containing compartment. In some embodiments, all of the cell-containing compartments are surrounded by a single barrier compartment. In some embodiments, the barrier compartment is substantially cell-free.

In an embodiment, cells to be incorporated into a device described herein, e.g., a hydrogel capsule, are prepared in the form of a cell suspension prior to being encapsulated within the device. The cells in the suspension may take the form of single cells (e.g., from a monolayer cell culture), or provided in another form, e.g., disposed on a microcarrier (e.g., a bead or matrix) or as a three-dimensional aggregate of cells (e.g., a cell cluster or spheroid). The cell suspension can comprise multiple cell clusters (e.g., as spheroids) or microcarriers.

In an embodiment, the implantable element described herein comprises a plurality of engineered mammalian cells (e.g., engineered ARPE-19 cells), e.g., at a particular cell density. For example, the implantable element may comprise a plurality of engineered mammalian cells at greater than 1 million cells per mL, 2.5 million cells per mL, 5 million cells per mL, 7.5 million cells per mL, 10 million cells per mL, 15 million cells per mL, 20 million cells per mL, 25 million cells per mL, 30 million cells per mL, 40 million cells per mL, 50 million cells per mL, or more. In an embodiment, the implantable element comprises a plurality of engineered ARPE-19 cells capable of expressing a protein (e.g., a hormone, a blood clotting factor, an antibody, or an enzyme) with a cell density of between 1-5 million cells per mL, 5-10 million cells per mL, or 10-20 million cells per mL. In an embodiment, the implantable element comprises a plurality of engineered ARPE-19 cells capable of expressing a protein (e.g., insulin) with a cell density of between 1-5 million cells per mL, 5-10 million cells per mL, or 10-20 million cells per mL. In an embodiment, the implantable element comprises a plurality of engineered ARPE-19 cells capable of expressing a protein (e.g., IDUA) with a cell density of between 1-5 million cells per mL, 5-10 million cells per mL, or 10-20 million cells per mL. In an embodiment, the implantable element comprises a plurality of engineered ARPE-19 cells capable of expressing a protein (e.g., insulin) with a cell density of between 1-5 million cells per mL, 5-10 million cells per mL, or 10-20 million cells per mL. In an embodiment, the implantable element comprises a plurality of engineered ARPE-19 cells capable of expressing a protein (e.g., insulin) with a cell density of between 1-5 million cells per mL, 5-10 million cells per mL, or 10-20 million cells per mL.

A device (e.g., capsule, particle) may comprise one or more exogenous agents that are not expressed by the cells, and may include, e.g., a nucleic acid (e.g., an RNA or DNA molecule), a protein (e.g., a hormone, an enzyme (e.g., glucose oxidase, kinase, phosphatase, oxygenase, hydrogenase, reductase) antibody, antibody fragment, antigen, or epitope)), an active or inactive fragment of a protein or polypeptide, a small molecule, or drug. In an embodiment, the device is configured to release such an exogenous agent.

In an embodiment, the implantable element described herein results in a lower amount of pericapsular fibrotic overgrowth (PFO) when implanted into a mammalian host than compared with implanting a control implantable element (e.g., defined as an otherwise identical implantable element except that the cell does not have the reduction in the MHC class I complex). In an embodiment, the implantable element described herein comprises an engineered mammalian cell that remains capable of expressing the therapeutic agent for at least any of two months, three months, four months, or longer following implant of the implantable element into a mammalian subject. In an embodiment, the implantable element described comprises an engineered mammalian cell expressing a therapeutic agent detectable in the plasma of a mammalian subject for at least any of two months, three months, four months, or longer following implant of the implantable element into the subject.

### Afibrotic (e.g., FBR-Mitigating) Compounds

In some embodiments, the devices described herein comprise at least one compound of Formula (I):

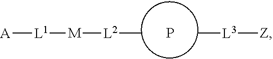

or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₁-C₆-alkenylene)-, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is absent, cycloalkyl, heterocyclyl, or heteroaryl, each of which is optionally substituted by one or more R⁴;
Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})CFv^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶.
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1}), cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

In some embodiments, the compound of Formula (I) is a compound of Formula (I-a):

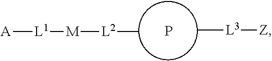

or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D})—N(R^{C})C(O)(C₁-C₆- alkylene)-, —N(R^{C})C(O)(C₁-C₆-alkenylene)-, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is heteroaryl optionally substituted by one or more R⁴;
Z is alkyl, alkenyl, alkynyl, heteroalkyl, cycloalkyl, heterocyclyl, aryl, or heteroaryl, each of which is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶; each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is2, 3, or 4.

In some embodiments, for Formulas (I) and (I-a), A is alkyl, alkenyl, alkynyl, heteroalkyl, cycloalkyl, heterocyclyl, aryl, heteroaryl, —O—, —C(O)O—, —C(O)—, —OC(O)—, —N(R^{C})C(O)—, —N(R^{C})C(O)(C₁-C₆-alkylene)-, —N(R^{C})C(O)(C₁-C₆-alkenylene)-, or —N(R^{C})—. In some embodiments, A is alkyl, alkenyl, alkynyl, heteroalkyl, cycloalkyl, heterocyclyl, aryl, heteroaryl, —O—, —C(O)O—, —C(O)—, —OC(O)—, or —N(R^{C})—. In some embodiments, A is alkyl, alkenyl, alkynyl, heteroalkyl, —O—, —C(O)O—, —C(O)—, —OC(O)—, or N(R^{C})—. In some embodiments, A is alkyl, —O—, —C(O)O—, —C(O)—, —OC(O), or —N(R^{C})—. In some embodiments, A is —N(R^{C})C(O)—, —N(R^{C})C(O)(C₁-C₆-alkylene)-, or —N(R^{C})C(O)(C₁-C₆-alkenylene)-. In some embodiments, A is —N(R^{C})—. In some embodiments, A is —N(R^{C})—, and R^{C} an R^{D} is independently hydrogen or alkyl. In some embodiments, A is —NH—. In some embodiments, A is —N(R^{C})C(O)(C₁-C₆-alkylene)-, wherein alkylene is substituted with R¹. In some embodiments, A is —N(R^{C})C(O)(C₁-C₆-alkylene)-, and Rt is alkyl (e.g., methyl). In some embodiments, A is —NHC(O)C(CH₃)₂—. In some embodiments, A is —N(R^{C})C(O)(methylene)-, and R¹ is alkyl (e.g., methyl). In some embodiments, A is —NHC(O)CH(CH₃)—. In some embodiments, A is —NHC(O)C(CH₃)—.

In some embodiments, for Formulas (I) and (I-a), L¹ is a bond, alkyl, or heteroalkyl. In some embodiments, L¹ is a bond or alkyl. In some embodiments, L¹ is a bond. In some embodiments, L¹ is alkyl. In some embodiments, L¹ is C₁-C₆ alkyl. In some embodiments, L¹ is —CH₂—, —CH(CH₃)—, —CH₂CH₂CH₂, or —CH₂CH₂—. In some embodiments, L¹ is —CH₂— or —CH₂CH₂—.

In some embodiments, for Formulas (I) and (I-a), L³ is a bond, alkyl, or heteroalkyl. In some embodiments, L³ is a bond. In some embodiments, L³ is alkyl. In some embodiments, L³ is C₁-C₁₂ alkyl. In some embodiments, L³ is C₁-C₆ alkyl. In some embodiments, L³ is —CH₂—. In some embodiments, L³ is heteroalkyl. In some embodiments, L³ is C₁-C₁₂ heteroalkyl, optionally substituted with one or more R² (e.g., oxo). In some embodiments, L³ is C₁-C₆ heteroalkyl, optionally substituted with one or more R² (e.g., oxo). In some embodiments, L³ is —C(O)OCH₂—, —CH₂(OCH₂CH₂)₂—, —CH₂(OCH₂CH₂)₃—, CH₂CH₂O—, or —CH₂O—. In some embodiments, L³ is —CH₂O—.

In some embodiments, for Formulas (I) and (I-a), M is absent, alkyl, heteroalkyl, aryl, or heteroaryl. In some embodiments, for Formulas (I) and (I-a), M is absent, alkyl, heteroalkyl, aryl, or heteroaryl. In some embodiments, M is heteroalkyl, aryl, or heteroaryl. In some embodiments, M is absent. In some embodiments, M is alkyl (e.g., C₁-C₆ alkyl). In some embodiments, M is —CH₂—. In some embodiments, M is heteroalkyl (e.g., C₁-C₆ heteroalkyl). In some embodiments, M is (—OCH₂CH₂—)_{z}, wherein z is an integer selected from 1 to 10. In some embodiments, z is an integer selected from 1 to 5. In some embodiments, M is —(OCH₂)₂—, (—OCH₂CH₂—)₂, (—OCH₂CH₂—)₃, (—OCH₂CH₂—)₄, or (—OCH₂CH_{2 5}. In some embodiments, M is —OCH₂CH₂—, (—OCH₂CH₂—)₂, (—OCH₂CH₂—)₃, or (—OCH₂CH₂—)₄. In some embodiments, M is (—OCH₂—)₃. In some embodiments, M is aryl. In some embodiments, M is phenyl. In some embodiments, M is unsubstituted phenyl. In some embodiments, M is

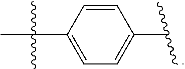 [SMILES: *c1ccc(*)cc1]

In some embodiments, M is

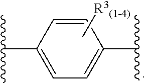

In some embodiments, M is phenyl substituted with 1-4 R³ (e.g., 1 R³). In some embodiments, R³ is CF₃.

In some embodiments, for Formulas (I) and (I-a), P is absent, heterocyclyl, or heteroaryl. In some embodiments, for Formulas (I) and (I-a), P is absent, heterocyclyl, or heteroaryl. In some embodiments, P is absent. In some embodiments, for Formulas (I) and (I-a), P is a tricyclic, bicyclic, or monocyclic heteroaryl. In some embodiments, P is a monocyclic heteroaryl. In some embodiments, P is a nitrogen-containing heteroaryl. In some embodiments, P is a monocyclic, nitrogen-containing heteroaryl. In some embodiments, P is a 5-membered heteroaryl. In some embodiments, P is a 5-membered nitrogen-containing heteroaryl. In some embodiments, P is tetrazolyl, imidazolyl, pyrazolyl, or triazolyl, or pyrrolyl. In some embodiments, P is imidazolyl. In some embodiments, P is 1,2,3-triazolyl. In some embodiments, P is

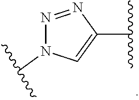 [SMILES: *c1cn(*)nn1]

In some embodiments, P is

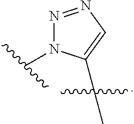 [SMILES: *c1cnnn1*]

In some embodiments, P is

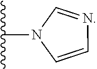 [SMILES: *n1ccnc1]

In some embodiments, P is heterocyclyl. In some embodiments, P is heterocyclyl. In some embodiments, P is a 5-membered heterocyclyl. In some embodiments, P is imidazolidinonyl. In some embodiments, P is

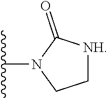 [SMILES: *N1CCNC1=O]

In some embodiments, P is thiomorpholinyl-1,1-dioxidyl.

In some embodiments, P is

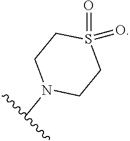 [SMILES: *N1CCS(=O)(=O)CC1]

In some embodiments, for Formulas (I) and (I-a), Z is alkyl, heteroalkyl, cycloalkyl, heterocyclyl, aryl, or heteroaryl. In some embodiments, for Formulas (I) and (I-a), Z is alkyl, heteroalkyl, cycloalkyl, heterocyclyl, aryl, or heteroaryl. In some embodiments, Z is heterocyclyl. In some embodiments, Z is monocyclic or bicyclic heterocyclyl, 5-membered heterocyclyl, or 6-membered heterocyclyl. In some embodiments, Z is a 6-membered oxygen-containing heterocyclyl. In some embodiments, Z is tetrahydropyranyl. In some embodiments, Z is

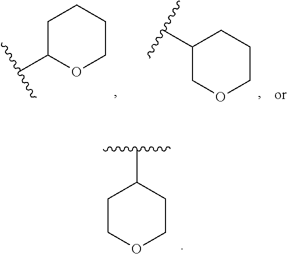 [SMILES: *C1CCCCO1 ; *C1CCCOC1 ; *C1CCOCC1]

In some embodiments, Z is a 4-membered oxygen-containing heterocyclyl. In some embodiments, Z is

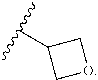 [SMILES: *C1COC1]

In some embodiments, Z is a bicyclic oxygen-containing heterocyclyl. In some embodiments, Z is a bicyclic oxygen-containing heterocyclyl. In some embodiments, Z is phthalic anhydridyl. In some embodiments, Z is a sulfur-containing heterocyclyl

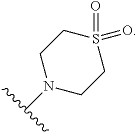 [SMILES: *N1CCS(=O)(=O)CC1]

In some embodiments, Z is a 6-membered sulfur-containing heterocyclyl

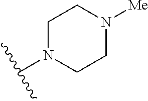 [SMILES: *N1CCN(C)CC1]

In some embodiments, Z is a 6-membered heterocyclyl containing a nitrogen atom and a sulfur atom. In some embodiments, Z is thiomorpholinyl-1,1-dioxidyl. In some embodiments, Z is

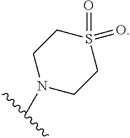 [SMILES: *N1CCS(=O)(=O)CC1]

In some embodiments, Z is a nitrogen-containing heterocyclyl. In some embodiments, Z is a 6-membered nitrogen-containing heterocyclyl. In some embodiments, Z is

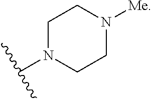 [SMILES: *N1CCN(C)CC1]

In some embodiments, Z is a bicyclic heterocyclyl. In some embodiments, Z is a bicyclic heterocyclyl. In some embodiments, Z is a bicyclic nitrogen-containing heterocyclyl, optionally substituted with one or more R⁵. In some embodiments, Z is 2-oxa-7-azaspiro[3.5]nonanyl

In some embodiments, Z is

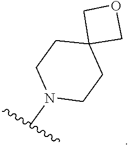 [SMILES: *N1CCC2(CC1)COC2]

In some embodiments, Z is 1-oxa-3,8-diazaspiro[4.5]decan-2-one. In some embodiments, Z is

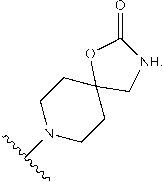 [SMILES: *N1CCC2(CC1)CNC(=O)O2]

In some embodiments, for Formulas (I) and (I-a), Z is aryl. In some embodiments, Z is monocyclic aryl. In some embodiments, Z is phenyl. In some embodiments, Z is monosubstituted phenyl (e.g., with 1 R⁵). In some embodiments, Z is monosubstituted phenyl, wherein the 1 R⁵ is a nitrogen-containing group. In some embodiments, Z is monosubstituted phenyl, wherein the 1 R⁵ is NH₂. In some embodiments, Z is monosubstituted phenyl, wherein the 1 R⁵ is an oxygen-containing group. In some embodiments, Z is monosubstituted phenyl, wherein the 1 R⁵ is an oxygen-containing heteroalkyl. In some embodiments, Z is monosubstituted phenyl, wherein the 1 R⁵ is OCH₃. In some embodiments, Z is monosubstituted phenyl, wherein the 1 R⁵ is in the ortho position. In some embodiments, Z is monosubstituted phenyl, wherein the 1 R⁵ is in the meta position. In some embodiments, Z is monosubstituted phenyl, wherein the 1 R⁵ is in the para position.

In some embodiments, for Formulas (I) and (I-a), Z is alkyl. In some embodiments, Z is C₁-C₁₂ alkyl. In some embodiments, Z is C₁-C₁₀ alkyl. In some embodiments, Z is C₁-C₈ alkyl. In some embodiments, Z is C₁-C₈ alkyl substituted with 1-5 R⁵. In some embodiments, Z is C₁-C₈ alkyl substituted with 1 R⁵. In some embodiments, Z is C₁-C₈ alkyl substituted with 1 R⁵, wherein R⁵ is alkyl, heteroalkyl, halogen, oxo, —OR^{A1}, —C(O)OR^{A1}, —C(O)R^{B1}, —OC(O)R^{B1}, or —N(R^{C1})(R^{D1}). In some embodiments, Z is C₁-C₈ alkyl substituted with 1 R⁵, wherein R⁵ is —OR^{A1} or —C(O)OR^{A1}. In some embodiments, Z is C₁-C₈ alkyl substituted with 1 R⁵, wherein R⁵ is —OR^{A1} or —C(O)OH. In some embodiments, Z is —CH₃.

In some embodiments, for Formulas (I) and (I-a), Z is heteroalkyl. In some embodiments, Z is C₁-C₁₂ heteroalkyl. In some embodiments, Z is C₁-C₁₀ heteroalkyl. In some embodiments, Z is C₁-C₈ heteroalkyl. In some embodiments, Z is C₁-C₆ heteroalkyl. In some embodiments, Z is a nitrogen-containing heteroalkyl optionally substituted with one or more R⁵. In some embodiments, Z is a nitrogen and sulfur-containing heteroalkyl substituted with 1-5 R⁵. In some embodiments, Z is N-methyl-2-(methylsulfonyl)ethan-1-aminyl.

In some embodiments, Z is —OR^{A} or —C(O)OR^{A}. In some embodiments, Z is —OR^{A} (e.g., —OH or —OCH₃). In some embodiments, Z is —OCH₃. In some embodiments, Z is —C(O)OR^{A} (e.g., —C(O)OH).

In some embodiments, Z is hydrogen.

In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound of Formula (I) is a compound of Formula (I-b):

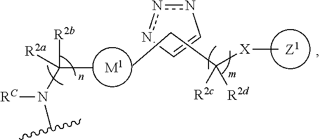

or a pharmaceutically acceptable salt thereof, wherein Ring M¹ is cycloalkyl, heterocyclyl, aryl, or heteroaryl, each of which is optionally substituted with 1-5 R³; Ring Z¹ is cycloalkyl, heterocyclyl, aryl or heteroaryl, optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl, or each of R^{2a} and R^{2b} or R^{2c} and R^{2d} is taken together to form an oxo group; X is absent, N(R¹⁰)(R¹¹), O, or S, R^{C} is hydrogen, alkyl, alkenyl, alkynyl, heteroalkyl, cycloalkyl, heterocyclyl, aryl, or heteroaryl, wherein each of alkyl, alkenyl, alkynyl, heteroalkyl, cycloalkyl, heterocyclyl, aryl, or heteroaryl is optionally substituted with 1-6 R⁶; each R³, R⁵ and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each of R¹⁰ and R¹¹ is independently hydrogen, alkyl, alkenyl, alkynyl, heteroalkyl, —C(O)OR^{A1}, —C(O)R^{B1}, —OC(O)R^{B1}, —C(O)N(R^{C1}), cycloalkyl, heterocyclyl or heteroaryl; each R^{A1}, R^{B1}, R^{C}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; each m and n is independently 1, 2, 3, 4, 5, or 6; and “ [symbol] ” refers to a connection to an attachment group or a polymer described herein. In some embodiments, for each R³ and R⁵, each alkyl, alkenyl, alkynyl, heteroalkyl, cycloalkyl, heterocyclyl, aryl, or heteroaryl is optionally and independently substituted with halogen, oxo, cyano, cycloalkyl, or heterocyclyl.

In some embodiments, the compound of Formula (I-b) is a compound of Formula (I-b-i):

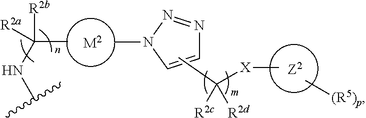

or a pharmaceutically acceptable salt thereof, wherein Ring M² is aryl or heteroaryl optionally substituted with one or more R³; Ring Z² is cycloalkyl, heterocyclyl, aryl, or heteroaryl; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; X is absent, O, or S; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; or two R⁵ are taken together to form a 5-6 membered ring fused to Ring Z²; each R^{A1} and R^{B1} is independently hydrogen, alkyl, or heteroalkyl; m and n are each independently 1, 2, 3, 4, 5, or 6; p is 0, 1, 2, 3, 4, 5, or 6, and “ [symbol] ” refers to a connection to an attachment group or a polymer described herein.

In some embodiments, the compound of Formula (I-b-i) is a compound of Formula (I-b-ii):

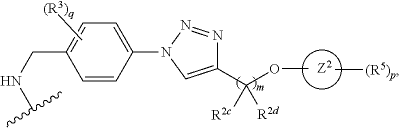

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R and taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-c):

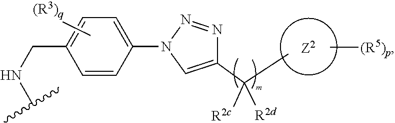

or a pharmaceutically acceptable salt thereof, wherein Ring Z² is cycloalkyl, heterocyclyl aryl or heteroaryl; each of R^{2c} and R^{2d} is independently hydrogen, alkyl, or heteroalkyl, or R^{2c} and R^{2d} is taken together to form an oxo group; each R³ and R⁵ is independently alkyl, heteroalkyl, halogen, oxo, —OR, —C(O)OR, or —C(O)R^{B1}, wherein each alkyl and heteroalkyl is optionally substituted with halogen; each R^{A1} and R^{B1} is independently hydrogen, alkyl, or heteroalkyl; m is 1, 2, 3, 4, 5, or 6; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-d):

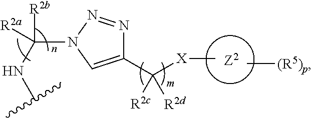

or a pharmaceutically acceptable salt thereof, wherein Ring Z² is cycloalkyl, heterocyclyl aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-e):

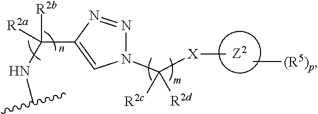

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-f):

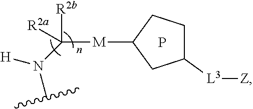

or a pharmaceutically acceptable salt thereof, wherein M is alkyl optionally substituted with one or more R³; Ring P is heteroaryl optionally substituted with one or more R⁴; L³ is alkyl or heteroalkyl optionally substituted with one or more R²; Z is alkyl, heteroalkyl, cycloalkyl, heterocyclyl, aryl, or heteroaryl, each of which is optionally substituted with one or more R⁵; each of R^{2a} and R^{2b} is independently hydrogen, alkyl, or heteroalkyl, or R^{2a} and R^{2b} is taken together to form an oxo group; each R², R³, R⁴, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (II):

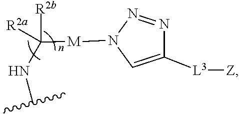

or a pharmaceutically acceptable salt thereof, wherein M is a bond, alkyl or aryl, wherein alkyl and aryl is optionally substituted with one or more R³; L³ is alkyl or heteroalkyl optionally substituted with one or more R²; Z is hydrogen, alkyl, heteroalkyl, cycloalkyl, heterocyclyl, aryl, heteroaryl or —OR, wherein alkyl, cycloalkyl, heterocyclyl, aryl, and heteroaryl is optionally substituted with one or more R⁵; R^{A} is hydrogen; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (II) is a compound of Formula (II-a):

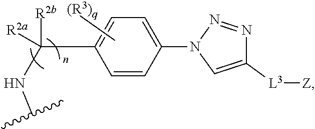

or a pharmaceutically acceptable salt thereof, wherein L³ is alkyl or heteroalkyl, each of which is optionally substituted with one or more R²; Z is hydrogen, alkyl, heteroalkyl or —OR^{A}, heteroalkyl are optionally substituted with one or more R⁵; each of R^{2a} and R^{2b} is independently hydrogen, alkyl, or heteroalkyl, or each of R² and R^{2b} is taken together to form an oxo group each R², R³, and R⁵ is independently heteroalkyl, halogen, oxo, —OR^{A1}, —C(O)OR^{A1}; R^{A} is hydrogen; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (III):

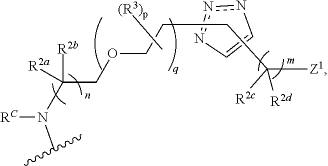

or a pharmaceutically acceptable salt thereof, wherein Z¹ is alkyl, alkenyl, alkynyl, heteroalkyl, cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl; or each of R^{2a} and R^{2b} or R^{2c} and R^{2d} is taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; q is an integer from 0 to 25; and “ [symbol] ” refers to a connection to an attachment group or a polymer described herein.

In some embodiments, the compound of Formula (III) is a compound of Formula (III-a):

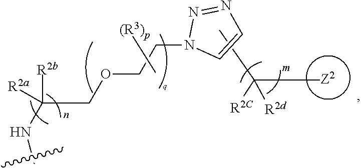

or a pharmaceutically acceptable salt thereof, wherein Ring Z² is cycloalkyl, heterocyclyl, aryl, or heteroaryl; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; and “ [symbol] ” refers to a connection to an attachment group or a polymer described herein.

In some embodiments, the compound of Formula (III-a) is a compound of Formula (III-b):

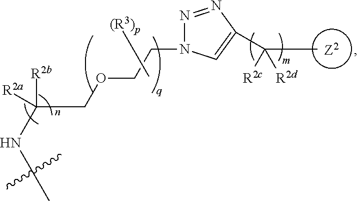

or a pharmaceutically acceptable salt thereof, wherein Ring Z² is cycloalkyl, heterocyclyl, aryl, or heteroaryl, each of which is optionally substituted with 1-5 R⁵ each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n 1, 2, 3, 4, 5, or 6; o and p are each independently 0, 1, 2, 3, 4, or 5; q is an integer from 0 to 25; and “ [symbol] ” refers to a connection to an attachment group or a polymer described herein.

In some embodiments, the compound of Formula (III-a) is a compound of Formula (III-c):

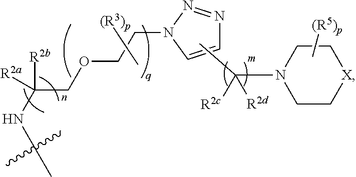

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4; q is an integer from 0 to 25; x is 0, 1, or 2; and “ [symbol] ” refers to a connection to an attachment group or a polymer described herein.

In some embodiments, the compound of Formula (III-c) is a compound of Formula (III-d):

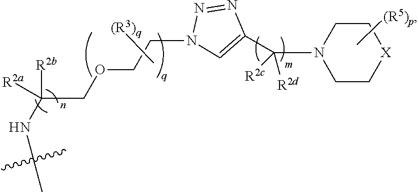

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{1d} is independently hydrogen, alkyl, heteroalkyl, or halo; or R^{2a} and R^{2b} or R^{2c} and R^{1d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4; q is an integer from 0 to 25; x is 0, 1, or 2; and “ [symbol] ” refers to a connection to an attachment group or a polymer described herein.

In some embodiments, the compound is a compound of Formula (I). In some embodiments, L² is a bond and P and L³ are independently absent.

In some embodiments, the compound is a compound of Formula (I-a). In some embodiments of Formula (II-a), L² is a bond, P is heteroaryl, L³ is a bond, and Z is hydrogen. In some embodiments, P is heteroaryl, L³ is heteroalkyl, and Z is alkyl. In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound is a compound of Formula (I-b). In some embodiments, P is absent, L¹ is —NHCH₂, L² is a bond, M is aryl (e.g., phenyl), L³ is —CH₂O, and Z is heterocyclyl (e.g., a nitrogen-containing heterocyclyl, e.g., thiomorpholinyl-1,1-dioxide). In some embodiments, the compound of Formula (I-b) is Compound 116.

In some embodiments of Formula (I-b), P is absent, L¹ is —NHCH₂, L² is a bond, M is absent, L³ is a bond, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-b) is Compound 105.

In some embodiments, the compound is a compound of Formula (I-b-i). In some embodiments of Formula (I-b-i), each of R^{2a} and R^{2b} is independently hydrogen or CH₃, each of R^{2c} and R^{2d} is independently hydrogen, m is 1 or 2, n is 1, X is O, p is 0, M² is phenyl optionally substituted with one or more R³, R³ is —CF₃, and Z² is heterocyclyl (e.g., an oxygen-containing heterocyclyl, e.g., tetrahydropyranyl, tetrahydrofuranyl, oxetanyl, or oxiranyl). In some embodiments, the compound of Formula (I-b-i) is Compound 100, Compound 106, Compound 107, Compound 108, Compound 109, or Compound 111.

In some embodiments, the compound is a compound of Formula (I-b-ii). In some embodiments of Formula (I-b-ii), each of R^{2a}, R^{2b}, R^{2c}, and R^{2d} is independently hydrogen, q is 0, p is 0, m is 1, and Z² is heterocyclyl (e.g., an oxygen-containing heterocyclyl, e.g., tetrahydropyranyl). In some embodiments, the compound of Formula (I-b-ii) is Compound 100.

In some embodiments, the compound is a compound of Formula (I-c). In some embodiments of Formula (I-c), each of R^{2c} and R^{2d} is independently hydrogen, m is 1, p is 1, q is 0, R⁵ is —CH₃, and Z is heterocyclyl (e.g., a nitrogen-containing heterocyclyl, e.g., piperazinyl). In some embodiments, the compound of Formula (I-c) is Compound 113.

In some embodiments, the compound is a compound of Formula (I-d). In some embodiments of Formula (I-d), each of R^{2a}, R^{2b}, R^{2c}, and R^{2d} is independently hydrogen, m is 1, n is 3, X is O, p is 0, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-d) is Compound 110 or Compound 114.

In some embodiments, the compound is a compound of Formula (I-f). In some embodiments of Formula (I-f), each of R^{2a} and R^{2b} is independently hydrogen, n is 1, M is —CH₂—, P is a nitrogen-containing heteroaryl (e.g., imidazolyl), L³ is —C(O)OCH₂—, and Z is CH₃. In some embodiments, the compound of Formula (I-f) is Compound 115.

In some embodiments, the compound is a compound of Formula (II-a). In some embodiments of Formula (II-a), each of R^{2a} and R^{2b} is independently hydrogen, n is 1, q is 0, L³ is —CH₂(OCH₂CH₂)₂, and Z is —OCH₃. In some embodiments, the compound of Formula (II-a) is Compound 112.

In some embodiments of Formula (II-a), each of R^{2a} and R^{2b} is independently hydrogen,
n is 1, L³ is a bond or —CH₂, and Z is hydrogen or —OH. In some embodiments, the compound of Formula (II-a) is Compound 103 or Compound 104.

In some embodiments, the compound is a compound of Formula (III). In some embodiments of Formula (III), each of R^{2a}, R^{2b}, R^{2c}, and R^{2d} is independently hydrogen, m is 1, n is 2, q is 3, p is 0, R^{C} is hydrogen, and Z¹ is heteroalkyl optionally substituted with R⁵ (e.g., —N(CH₃)(CH₂CH₂)S(O)₂CH₃). In some embodiments, the compound of Formula (III) is Compound 120.

In some embodiments, the compound is a compound of Formula (III-b). In some embodiments of Formula (III-b), each of R^{2a}, R^{2b}, R^{2c}, and R^{2d} is independently hydrogen, m is 0, n is 2, q is 3, p is 0, and Z² is aryl (e.g., phenyl) substituted with 1 R⁵ (e.g., —NH₂). In some embodiments, the compound of Formula (III-b) is Compound 102.

In some embodiments, the compound is a compound of Formula (III-b). In some embodiments of Formula (III-b), each of R^{2a}, R^{2b}, R^{2c}, and R^{2d} is independently hydrogen, m is 1, n is 2, q is 3, p is 0, R^{C} is hydrogen, and Z² is heterocyclyl (e.g., a nitrogen-containing heterocyclyl, e.g., a nitrogen-containing Spiro heterocyclyl, e.g., 2-oxa-7-azaspiro[3.5]nonanyl). In some embodiments, the compound of Formula (III-b) is Compound 121.

In some embodiments, the compound is a compound of Formula (III-d). In some embodiments of Formula (III-d), each of R^{2a}, R^{2b}, R^{2c}, and R^{2d} is independently hydrogen, m is 1, n is 2, q is 1, 2, 3, or 4, p is 0, and X is S(O)₂. In some embodiments of Formula (III-d), each of R^{2a} and R^{2b} is independently hydrogen, m is 1, n is 2, q is 1, 2, 3, or 4, p is 0, and X is S(O)₂.

In some embodiments, the compound of Formula (III-d) is Compound 101, Compound 117, Compound 118, or Compound 119.

In some embodiments, the compound is a compound of Formula (I-b), (I-d), or (I-e). In some embodiments, the compound is a compound of Formula (I-b), (I-d), or (II). In some embodiments, the compound is a compound of Formula (I-b), (I-d), or (I-f). In some embodiments, the compound is a compound of Formula (I-b), (I-d), or (III).

In some embodiments, the compound of Formula (I) is not a compound disclosed in WO2012/112982, WO2012/167223, WO2014/153126, WO2016/019391, WO 2017/075630, US2012-0213708, US 2016-0030359 or US 2016-0030360.

In some embodiments, the compound of Formula (I) comprises a compound shown in Table 4, or a pharmaceutically acceptable salt thereof. In some embodiments, the exterior surface and/or one or more compartments within a device described herein comprises a small molecule compound shown in Table 4, or a pharmaceutically acceptable salt thereof.

TABLE 4
Exemplary afibrotic (FBR-mitigating) compounds

| Compound No. | Structure |
|---|---|
| 100 | 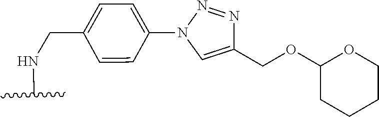 [SMILES: *NCc1ccc(-n2cc(COC3CCCCO3)nn2)cc1] |
| 101 | 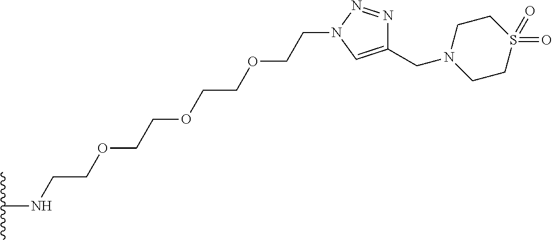 [SMILES: *NCCOCCOCCOCCn1cc(CN2CCS(=O)(=O)CC2)nn1] |
| 102 | 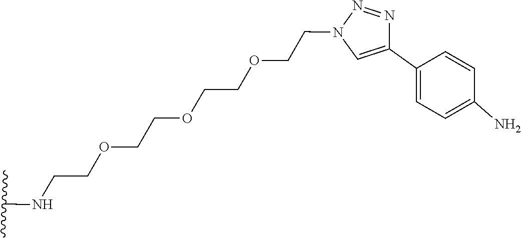 [SMILES: *NCCOCCOCCOCCn1cc(-c2ccc(N)cc2)nn1] |
| 103 | 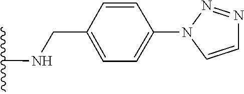 [SMILES: *NCc1ccc(-n2ccnn2)cc1] |
| 104 | 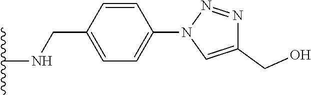 [SMILES: *NCc1ccc(-n2cc(CO)nn2)cc1] |
| 105 | 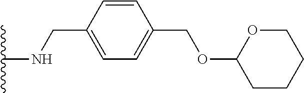 [SMILES: *NCc1ccc(COC2CCCCO2)cc1] |
| 106 | 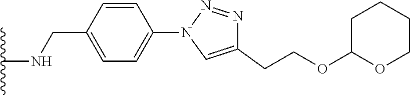 [SMILES: *NCc1ccc(-n2cc(CCOC3CCCCO3)nn2)cc1] |
| 107 | 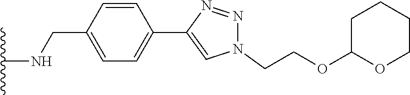 [SMILES: *NCc1ccc(-c2cn(CCOC3CCCCO3)nn2)cc1] |
| 108 | 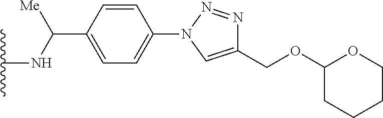 [SMILES: *NC(C)c1ccc(-n2cc(COC3CCCCO3)nn2)cc1] |
| 109 | 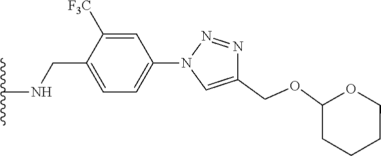 [SMILES: *NCc1ccc(-n2cc(COC3CCCCO3)nn2)cc1C(F)(F)F] |
| 110 | 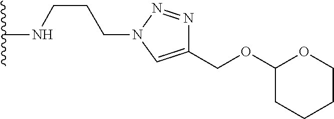 [SMILES: *NCCCn1cc(COC2CCCCO2)nn1] |
| 111 | 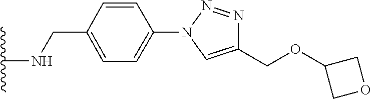 [SMILES: *NCc1ccc(-n2cc(COC3COC3)nn2)cc1] |
| 112 | 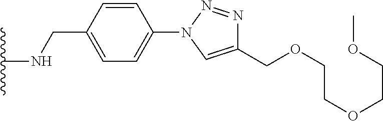 [SMILES: *NCc1ccc(-n2cc(COCCOCCOC)nn2)cc1] |
| 113 | 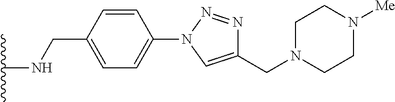 [SMILES: *NCc1ccc(-n2cc(CN3CCN(C)CC3)nn2)cc1] |
| 114 | 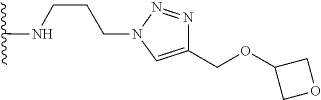 [SMILES: *NCCCn1cc(COC2COC2)nn1] |
| 115 | 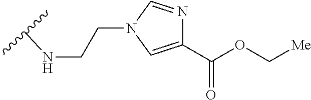 [SMILES: *NCCn1cnc(C(=O)OCC)c1] |
| 116 | 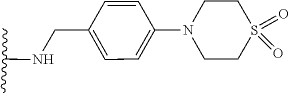 [SMILES: *NCc1ccc(N2CCS(=O)(=O)CC2)cc1] |
| 117 | 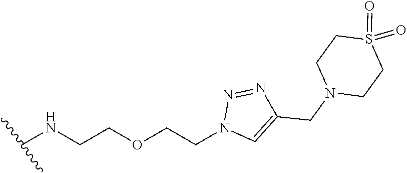 [SMILES: *NCCOCCn1cc(CN2CCS(=O)(=O)CC2)nn1] |
| 118 | 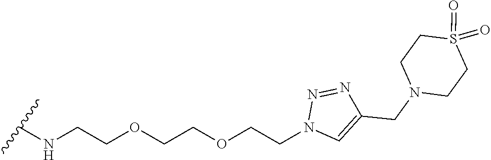 [SMILES: *NCCOCCOCCn1cc(CN2CCS(=O)(=O)CC2)nn1] |
| 119 | 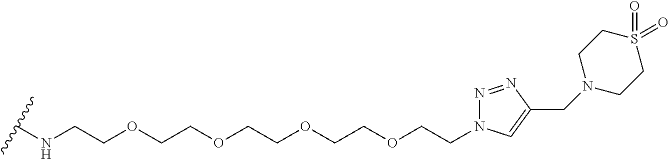 [SMILES: *NCCOCCOCCOCCOCCn1cc(CN2CCS(=O)(=O)CC2)nn1] |
| 120 | 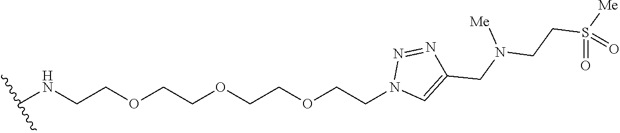 [SMILES: *NCCOCCOCCOCCn1cc(CN(C)CCS(C)(=O)=O)nn1] |
| 121 | 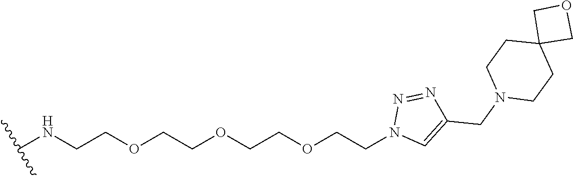 [SMILES: *NCCOCCOCCOCCn1cc(CN2CCC3(CC2)COC3)nn1] |
| 122 | 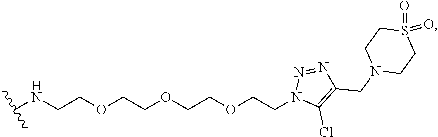 [SMILES: *NCCOCCOCCOCCn1nnc(CN2CCS(=O)(=O)CC2)c1Cl] |
| 123 | 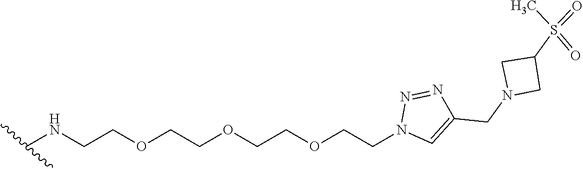 [SMILES: *NCCOCCOCCOCCn1cc(CN2CC(S(C)(=O)=O)C2)nn1] |

Conjugation of any of the compounds in Table 4 to a polymer (e.g., an alginate) may be performed as described in Example 2 of WO 2019/195055 or any other suitable chemical reaction.

In some embodiments, the compound is a compound of Formula (I) (e.g., Formulas (I-a), (I-b), (I-c), (I-d), (I-e), (I-f), (II), (II-a), (III), (III-a), (III-b), (III-c), or (III-d)), or a pharmaceutically acceptable salt thereof and is selected from:

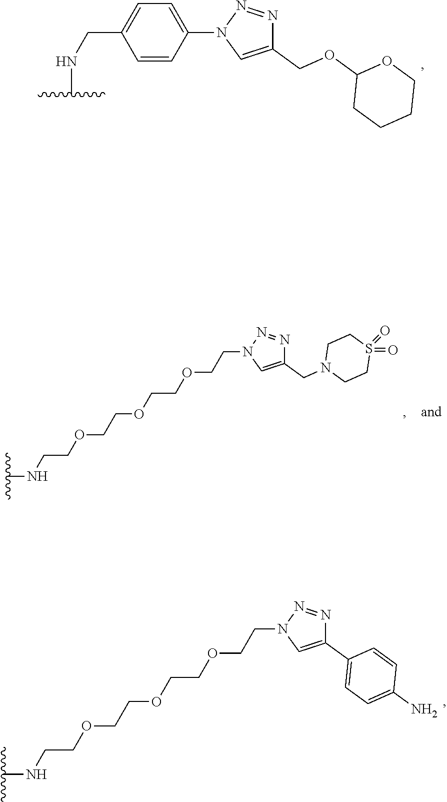 [SMILES: *NCc1ccc(-n2cc(COC3CCCCO3)nn2)cc1 ; *NCCOCCOCCOCCn1cc(CN2CCS(=O)(=O)CC2)nn1 ; *NCCOCCOCCOCCn1cc(-c2ccc(N)cc2)nn1]

or a pharmaceutically acceptable salt thereof.

In some embodiments, the device described herein comprises the compound of

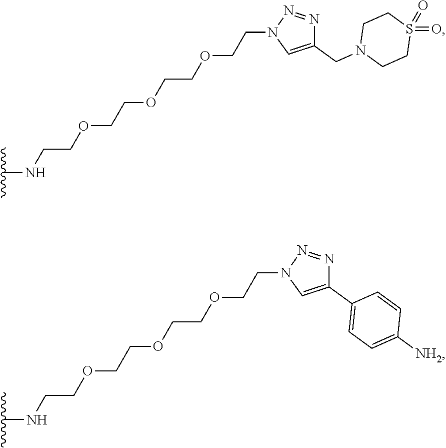 [SMILES: *NCCOCCOCCOCCn1cc(CN2CCS(=O)(=O)CC2)nn1 ; *NCCOCCOCCOCCn1cc(-c2ccc(N)cc2)nn1]

or a pharmaceutically acceptable salt of either compound.

In some embodiments, a compound of Formula (I) (e.g., Compound 101 in Table 4) is covalently attached to an alginate (e.g., an alginate with approximate MW<75 kDa, G:M ratio ≥1.5) at a conjugation density of at least 2.0% and less than 9.0%, or 3.0% to 8.0%, 4.0-7.0, 5.0 to 7.0, or 6.0 to 7.0 or about 6.8 as determined by combustion analysis for percent nitrogen as described in WO 2020/069429. In an embodiment, the conjugation density of Compound 101 in the modified alginate is determined by quantitative free amine analysis, e.g., as described in WO2020198695, wherein the determined conjugation density is 1.0% w/w to 3.0% w/w, 1.3% w/w to 2.8% w/w, 1.3% w/w to 2.6% w/w, 1.5% w/w to 2.4% w/w, 1.5% w/w to 2.2% w/w, or 1.7% w/w to 2.2% w/w.

A device, device preparation or device composition may be configured for implantation, or is implanted or disposed, into or onto any site or part of the body. In some embodiments, the implantable device or device preparation is configured for implantation into the peritoneal cavity (e.g., the lesser sac, also known as the omental bursa or bursalis omentum). A device, device preparation or device composition may be implanted in the peritoneal cavity (e.g., the omentum, e.g., the lesser sac) or disposed on a surface within the peritoneal cavity (e.g., omentum, e.g., lesser sac) via injection or catheter. Additional considerations for implantation or disposition of a device, device preparation or device composition into the omentum (e.g., the lesser sac) are provided in M. Pellicciaro et al. (2017) CellR4 5(3):e2410.

### Device Manufacture

Engineered mammalian cell (e.g., an engineered ARPE-19 cell)s for use in manufacturing a device, e.g., an implantable element, described herein may be generated and cultured using methods known in the art. For example, stably-transfected ARPE-19 cells may be cultured in vitro substantially as described in WO2020198695.

Compounds of Formula (I) and alginates modified with such compounds may be obtained using procedures known in the art, e.g., substantially as those described in WO2020198695.

Alginate solutions for making two-compartment hydrogel capsules may be obtained using procedures known in the art, e.g., substantially as described in WO2020198695.

Two-compartment hydrogel capsules encapsulating engineered mammalian cells described herein may be generated using procedure known in the art, e.g., substantially as described in WO2020198696.

### Methods of Treatment

Described herein are methods for preventing or treating a disease, disorder, or condition in a subject by administering to the subject an implantable element comprising an engineered mammalian cell (e.g., an engineered ARPE-19 cell), wherein the engineered mammalian cell comprises (i) a reduction in the level or function of a MHC class I protein complex, and optionally, a MHC class II protein complex and/or CIITA, and (ii) an exogenous nucleic acid encoding a therapeutic agent that, e.g., treats the disease, disorder or condition. In some embodiments, following administration, the engineered mammalian cell comprises a reduction in antigenicity or immunogenicity, e.g., as compared to an to an engineered mammalian cell that is substantially identical to or identical to the engineered mammalian cell except that it does not comprise a reduction in the level or function of a MHC class I protein complex, and optionally, a MHC class II protein complex and/or CIITA. In some embodiments, the reduction in antigenicity or immunogenicity comprises a reduction in the (a) release of particles or components of an engineered mammalian cell described herein into the subject's bloodstream, and/or (b) antigen presentation on the subject's cells comprising particles or components of engineered mammalian cells described herein, e.g., as compared to an to an engineered mammalian cell that is substantially identical to or identical to the engineered mammalian cell except that it does not comprise a reduction in the level or function of a MHC class I protein complex, and optionally, a MHC class II protein complex and/or CIITA. Such reductions can be characterized by standard methods known in the art, e.g., by obtaining a blood sample from a subject and quantifying, e.g., protein expression and/or RNA expression.

The cells may be administered by implanting into the subject an implantable element containing the cells as described herein, or a preparation of such devices. In an embodiment, the implantable element or preparation of implantable elements is implanted (e.g., via laparoscopy) into the intraperitoneal space, e.g., the greater sac of the peritoneal cavity. In an embodiment, the engineered mammalian cells are engineered RPE cells, and the method comprises administering (e.g., implanting) an effective amount of a composition of two-compartment alginate hydrogel capsules which comprise the engineered RPE cells and a cell-binding polymer described herein in the inner compartment and comprise a Compound of Formula (I), e.g., Compound 101, on the outer capsule surface. In some embodiments, the method of treatment directly or indirectly reduces or alleviates at least one symptom of the disease, disorder, or condition and/or the method prevents or slows the onset of the disease, disorder, or condition. In some embodiments, the subject is a human.

In some embodiments, the disease, disorder, or condition affects a system of the body, e.g., the nervous system (e.g., peripheral nervous system (PNS) or central nervous system (CNS)), vascular system, skeletal system, respiratory system, endocrine system, lymph system, reproductive system, or gastrointestinal tract. In some embodiments, the disease, disorder, or condition affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis.

In some embodiments, the disease, disorder or condition is a neurodegenerative disease, diabetes, a heart disease, an autoimmune disease, a cancer, a liver disease, a lysosomal storage disease, a blood clotting disorder or a coagulation disorder, an orthopedic condition, an amino acid metabolism disorder.

In some embodiments, the disease, disorder or condition is a neurodegenerative disease. Exemplary neurodegenerative diseases include Alzheimer's disease, Huntington's disease, Parkinson's disease (PD) amyotrophic lateral sclerosis (ALS), multiple sclerosis (MS) and cerebral palsy (CP), dentatorubro-pallidoluysian atrophy (DRPLA), neuronal intranuclear hyaline inclusion disease (NIHID), dementia with Lewy bodies, Down's syndrome, Hallervorden-Spatz disease, prion diseases, argyrophilic grain dementia, cortocobasal degeneration, dementia pugilistica, diffuse neurofibrillary tangles, Gerstmann-Straussler-Scheinker disease, Jakob-Creutzfeldt disease, Niemann-Pick disease type 3, progressive supranuclear palsy, subacute sclerosing panencephalitis, spinocerebellar ataxias, Pick's disease, and dentatorubral-pallidoluysian atrophy.

In some embodiments, the disease, disorder, or condition is an autoimmune disease, e.g., scleroderma, multiple sclerosis, lupus, or allergies.

In some embodiments, the disease is a liver disease, e.g., hepatitis B, hepatitis C, cirrhosis, NASH.

In some embodiments, the disease, disorder, or condition is cancer. Exemplary cancers include leukemia, lymphoma, melanoma, lung cancer, brain cancer (e.g., glioblastoma), sarcoma, pancreatic cancer, renal cancer, liver cancer, testicular cancer, prostate cancer, or uterine cancer.

In some embodiments, the disease, disorder, or condition is an orthopedic condition. Exemplary orthopedic conditions include osteoporosis, osteonecrosis, Paget's disease, or a fracture.

In some embodiments, the disease, disorder or condition is a lysosomal storage disease. Exemplary lysosomal storage diseases include Gaucher disease (e.g., Type I, Type II, Type III), Tay-Sachs disease, Fabry disease, Farber disease, Mucopolysaccharidosis type I (MPS I) (also known as Hurler syndrome), Hunter syndrome, lysosomal acid lipase deficiency, Niemann-Pick disease, Salla disease, Sanfilippo syndrome (also known as mucopolysaccharidosis type IIIA (MPS3A)), multiple sulfatase deficiency, Maroteaux-Lamy syndrome, metachromatic leukodystrophy, Krabbe disease, Scheie syndrome, Hurler-Scheie syndrome, Sly syndrome, hyaluronidase deficiency, Pompe disease, Danon disease, gangliosidosis, or Morquio syndrome.

In some embodiments, the disease, disorder, or condition is a blood clotting disorder or a coagulation disorder. Exemplary blood clotting disorders or coagulation disorders include hemophilia (e.g., hemophilia A or hemophilia B), Von Willebrand disease, thrombocytopenia, uremia, Bernard-Soulier syndrome, Factor XII deficiency, vitamin K deficiency, or congenital afibrinogenimia.

In some embodiments, the disease, disorder, or condition is an amino acid metabolism disorder, e.g., phenylketonuria, tyrosinemia (e.g., Type 1 or Type 2), alkaptonuria, homocystinuria, hyperhomocysteinemia, maple syrup urine disease.

In some embodiments, the disease, disorder, or condition is a fatty acid metabolism disorder, e.g., hyperlipidemia, hypercholesterolemia, galactosemia.

In some embodiments, the disease, disorder, or condition is a purine or pyrimidine metabolism disorder, e.g., Lesch-Nyhan syndrome.

In some embodiments, the disease, disorder, or condition is diabetes (e.g., Type I or Type II diabetes). In some embodiments, the disease, disorder or condition is not diabetes. In some embodiments, the disease, disorder or condition is not Type I diabetes. In some embodiments, the disease, disorder or condition is not Type II diabetes.

### ENUMERATED EMBODIMENTS

1. An implantable element comprising an engineered mammalian cell, wherein:
   (i) the engineered mammalian cell comprises a reduction in the level or function of a major histocompatibility complex (MHC) class I protein complex; and
   (ii) the engineered mammalian cell comprises an exogenous nucleic acid encoding a therapeutic agent.
2. The implantable element of embodiment 1, wherein the MHC class I protein complex comprises one or more of:
   (i) human leukocyte antigen (HLA) A;
   (ii) HLA-B;
   (iii) HLA-C; and
   (iv) beta-2-microglobulin (beta-2M).
3. The implantable element of embodiment 2, comprising (i).
4. The implantable element of any one of embodiments 2-3, comprising (ii).
5. The implantable element of any one of embodiments 2-4, comprising (iii).
6. The implantable element of any one of embodiments 2-5, comprising (iv).
7. The implantable element of any one of the preceding embodiments, wherein the engineered mammalian cell comprises a mutation resulting in the reduction of expression of a component of the MHC class I complex.
8. The implantable element of any one of the preceding embodiments, wherein the engineered mammalian cell comprises a lower-functioning or non-functioning variant of a component of the MHC class I component.
9. The implantable element of any one of the preceding embodiments, wherein expression of a component of the MHC class I complex is silenced or knocked down.
10. The implantable element of any one of the preceding embodiments, wherein the engineered mammalian cell comprises a reduction in the level of a MHC class I component by about 0.05%, 0.1%, 0.5%, 0.75%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the level of an MHC class I component.
11. The implantable element of any one of the preceding embodiments, wherein the engineered mammalian cell comprises a reduction in the level of the MHC class I component HLA-A by about 0.05%, 0.1%, 0.5%, 0.75%1, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the level of the MHC class I component HLA-A.
12. The implantable element of any one of the preceding embodiments, wherein the engineered mammalian cell comprises a reduction in the level of the MHC class I component HLA-B by about 0.05%, 0.1%, 0.5%, 0.75%1, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the level of the MHC class I component HLA-B.
13. The implantable element of any one of the preceding embodiments, wherein the engineered mammalian cell comprises a reduction in the level of the MHC class I component HLA-C by about 0.05%, 0.1%, 0.5%, 0.75%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the level of the MHC class I component HLA-C.
14. The implantable element of any one of the preceding embodiments, wherein the engineered mammalian cell comprises a reduction in the level of the MHC class I component beta-2M by about 0.05%, 0.1%, 0.5%, 0.75%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the level of the MHC class I component beta-2M.
15. The implantable element of any one of the preceding embodiments, wherein the engineered mammalian cell comprises a reduction in the level of a MHC class I component between 1-25%, 5-25%, 10-25%, 25-50%, 25-75%, or 50-75%, e.g., as compared to an engineered mammalian cell that is substantially identical to or identical to the engineered mammalian cell except that it does not comprise a reduction in the level of an MHC class I component.
16. The implantable element of any one of the preceding embodiments, wherein the engineered mammalian cell comprises a reduction in the level of a MHC class I component between 1-25%, e.g., as compared to an engineered mammalian cell that is substantially identical to or identical to the engineered mammalian cell except that it does not comprise a reduction in the level of an MHC class I component.
17. The implantable element of any one of the preceding embodiments, wherein the engineered mammalian cell comprises a reduction in the level of a MHC class I component between 5-25%, e.g., as compared to an engineered mammalian cell that is substantially identical to or identical to the engineered mammalian cell except that it does not comprise a reduction in the level of an MHC class I component.
18. The implantable element of any one of the preceding embodiments, wherein the engineered mammalian cell comprises a reduction in the level of a MHC class I component between 10-25%, e.g., as compared to an engineered mammalian cell that is substantially identical to or identical to the engineered mammalian cell except that it does not comprise a reduction in the level of an MHC class I component.
19. The implantable element of any one of the preceding embodiments, wherein the engineered mammalian cell comprises a reduction in the level of a MHC class I component between 25-50%, e.g., as compared to an engineered mammalian cell that is substantially identical to or identical to the engineered mammalian cell except that it does not comprise a reduction in the level of an MHC class I component.
20. The implantable element of any one of the preceding embodiments, wherein the engineered mammalian cell comprises a reduction in the level of a MHC class I component between 25-75%, e.g., as compared to an engineered mammalian cell that is substantially identical to or identical to the engineered mammalian cell except that it does not comprise a reduction in the level of an MHC class I component.
21. The implantable element of any one of the preceding embodiments, wherein the engineered mammalian cell comprises a reduction in the level of a MHC class I component between 50-75%, e.g., as compared to an engineered mammalian cell that is substantially identical to or identical to the engineered mammalian cell except that it does not comprise a reduction in the level of an MHC class I component.
22. The implantable element of any one of the preceding embodiments, wherein the engineered mammalian cell comprises a reduction in the level of a MHC class I component of greater than 50%, greater than 75%, or greater than 90%, e.g., as compared to an engineered mammalian cell that is substantially identical to or identical to the engineered mammalian cell except that it does not comprise a reduction in the level of an MHC class I component.
23. The implantable element of any one of the preceding embodiments, wherein the engineered mammalian cell comprises a reduction in the level of a MHC class I component of greater than 50%, e.g., as compared to an engineered mammalian cell that is substantially identical to or identical to the engineered mammalian cell except that it does not comprise a reduction in the level of an MHC class I component.
24. The implantable element of any one of the preceding embodiments, wherein the engineered mammalian cell comprises a reduction in the level of a MHC class I component of greater than 75%, e.g., as compared to an engineered mammalian cell that is substantially identical to or identical to the engineered mammalian cell except that it does not comprise a reduction in the level of an MHC class I component.
25. The implantable element of any one of the preceding embodiments, wherein the engineered mammalian cell comprises a reduction in the level of a MHC class I component of greater than 90%, e.g., as compared to an engineered mammalian cell that is substantially identical to or identical to the engineered mammalian cell except that it does not comprise a reduction in the level of an MHC class I component.
26. The implantable element of any one of the preceding embodiments, wherein the engineered mammalian cell comprises a reduction in the function of a MHC class I component by about 0.05%, 0.1%, 0.5%, 0.75%, 1%, 2%, 3%, 4%, 5%, 6%, 7%, 8%, 9%, 10%, 15%, 20%, 25%, 30%, 35%, 40%, 45, 50%, 55%, 60%, 65%, 70%, 75%, 80%, 85% 90%, 95%, 99%, or more, e.g., as compared to an engineered mammalian cell that is substantially identical to or identical to the engineered mammalian cell except that it does not comprise a reduction in the function of an MHC class I component.
27. The implantable element of any one of the preceding embodiments, wherein the engineered mammalian cell comprises a reduction in the function of HLA-A by about 0.05%, 0.1%, 0.5%, 0.75%, 1%, 2%, 3%, 4%, 5%, 6%, 7%, 8%, 9% 10% 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the function of HLA-A.
28. The implantable element of any one of the preceding embodiments, wherein the engineered mammalian cell comprises a reduction in the function of HLA-B by about 0.05%, 0.1%, 0.5%, 0.75%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the function of HLA-B.
29. The implantable element of any one of the preceding embodiments, wherein the engineered mammalian cell comprises a reduction in the function of HLA-C by about 0.05%, 0.1%, 0.5%, 0.75%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the function of HLA-C.
30. The implantable element of any one of the preceding embodiments, wherein the engineered mammalian cell comprises a reduction in the function of beta-2M by about 0.05%, 0.1%, 0.5%, 0.75%, 1%, 2%, 3%, 4%, 5%, 6%, 7%, 8%, 9%, 10%, 15% 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the function of beta-2M.
31. The implantable element of any one of the preceding embodiments, wherein the engineered mammalian cell comprises a reduction in the function of a MHC class I component between 1-25%, 5-25%, 10-25%, 25-50%, 25-75%, 50-75%, or 75-100%, e.g., as compared to an engineered mammalian cell that is substantially identical to or identical to the engineered mammalian cell except that it does not comprise a reduction in the function of an MHC class I component.
32. The implantable element of any one of the preceding embodiments, wherein the engineered mammalian cell comprises a reduction in the function of a MHC class I component between 1-25%, e.g., as compared to an engineered mammalian cell that is substantially identical to or identical to the engineered mammalian cell except that it does not comprise a reduction in the function of an MHC class I component.
33. The implantable element of any one of the preceding embodiments, wherein the engineered mammalian cell comprises a reduction in the function of a MHC class I component between 5-25%, e.g., as compared to an engineered mammalian cell that is substantially identical to or identical to the engineered mammalian cell except that it does not comprise a reduction in the function of an MHC class I component.
34. The implantable element of any one of the preceding embodiments, wherein the engineered mammalian cell comprises a reduction in the function of a MHC class I component between 10-25%, e.g., as compared to an engineered mammalian cell that is substantially identical to or identical to the engineered mammalian cell except that it does not comprise a reduction in the function of an MHC class I component.
35. The implantable element of any one of the preceding embodiments, wherein the engineered mammalian cell comprises a reduction in the function of a MHC class I component between 25-50%, e.g., as compared to an engineered mammalian cell that is substantially identical to or identical to the engineered mammalian cell except that it does not comprise a reduction in the function of an MHC class I component.
36. The implantable element of any one of the preceding embodiments, wherein the engineered mammalian cell comprises a reduction in the function of a MHC class I component between 25-75%, e.g., as compared to an engineered mammalian cell that is substantially identical to or identical to the engineered mammalian cell except that it does not comprise a reduction in the function of an MHC class I component.
37. The implantable element of any one of the preceding embodiments, wherein the engineered mammalian cell comprises a reduction in the function of a MHC class I component between 50-75%, e.g., as compared to an engineered mammalian cell that is substantially identical to or identical to the engineered mammalian cell except that it does not comprise a reduction in the function of an MHC class I component.
38. The implantable element of any one of the preceding embodiments, wherein the engineered mammalian cell comprises a reduction in the function of a MHC class I component between 75-100%, e.g., as compared to an engineered mammalian cell that is substantially identical to or identical to the engineered mammalian cell except that it does not comprise a reduction in the function of an MHC class I component.
39. The engineered mammalian cell of any one of the preceding embodiments, wherein the engineered mammalian cell comprises a reduction in the function of a MHC class I component of greater than 50%, greater than 75% or greater than 90%, e.g., as compared to an engineered mammalian cell that is substantially identical to or identical to the engineered mammalian cell except that it does not comprise a reduction in the function of an MHC class I component.
40. The engineered mammalian cell of any one of the preceding embodiments, wherein the engineered mammalian cell comprises a reduction in the function of a MHC class I component of greater than 50%, e.g., as compared to an engineered mammalian cell that is substantially identical to or identical to the engineered mammalian cell except that it does not comprise a reduction in the function of an MHC class I component.
41. The engineered mammalian cell of any one of the preceding embodiments, wherein the engineered mammalian cell comprises a reduction in the function of a MHC class I component of greater than 75%, e.g., as compared to an engineered mammalian cell that is substantially identical to or identical to the engineered mammalian cell except that it does not comprise a reduction in the function of an MHC class I component.
42. The engineered mammalian cell of any one of the preceding embodiments, wherein the engineered mammalian cell comprises a reduction in the function of a MHC class I component of greater than 90%, e.g., as compared to an engineered mammalian cell that is substantially identical to or identical to the engineered mammalian cell except that it does not comprise a reduction in the function of an MHC class I component.
43. The implantable element of any one of the preceding embodiments, wherein the engineered mammalian cell further comprises a reduction in the level or function of a MHC class II complex.
44. The implantable element of embodiment 43, wherein the MHC class II complex comprises one or more of:
   (v) human leukocyte antigen (HLA) DP;
   (vi) HLA-DM;
   (vii) HLA-DOA;
   (viii) HLA-DOB;
   (ix) HLA-DQ; and
   (x) HLA-DR.
45. The implantable element of embodiment 44, wherein the MHC class II complex comprises (v).
46. The implantable element of any one of embodiments 44-45, wherein the MHC class II complex comprises (vi).
47. The implantable element of any one of embodiments 44-46, wherein the MHC class II complex comprises (vii).
48. The implantable element of any one of embodiments 44-47, wherein the MHC class II complex comprises (viii).
49. The implantable element of any one of embodiments 44-48, wherein the MHC class II complex comprises (ix).
50. The implantable element of any one of embodiments 44-49, wherein the MHC class II complex comprises (x).
51. The implantable element of any one of embodiments 44-50, wherein the cell comprises a reduction in the function or expression of a class II major histocompatibility complex transactivator (CIITA).
52. The implantable element of any one of the preceding embodiments, wherein the engineered mammalian cell is a human cell.
53. The implantable element of any one of the preceding embodiments, wherein the engineered mammalian cell comprises an embryonic stem cell (ESC) or an induced pluripotent stem cell (iPSC).
54. The implantable element of any one of the preceding embodiments, wherein the engineered mammalian cell comprises an ESC.
55. The implantable element of any one of the preceding embodiments, wherein the engineered mammalian cell comprises an iPSC.
56. The implantable element of any one of the preceding embodiments, wherein the engineered mammalian cell comprises a retinal pigment epithelial (RPE) cell, a CCD-33Lu cell, a MRC-5 cell, a MRC-9 cell, a MCF10a cell, or a cell derived therefrom.
57. The implantable element of any one of the preceding embodiments, wherein the engineered mammalian cell comprises an engineered RPE cell (e.g., an engineered ARPE-19 cell).
58. The implantable element of any one of the preceding embodiments, wherein the engineered mammalian cell comprises a CCD-33Lu cell
59. The implantable element of any one of the preceding embodiments, wherein the engineered mammalian cell comprises a MRC-5 cell.
60. The implantable element of any one of the preceding embodiments, wherein the engineered mammalian cell comprises a MRC-9 cell.
61. The implantable element of any one of the preceding embodiments, wherein the engineered mammalian cell comprises a MCF10a cell.
62. The implantable element of any one of the preceding embodiments, wherein the exogenous nucleotide sequence is extrachromosomal.
63. The implantable element of any one of the preceding embodiments, wherein the exogenous nucleotide sequence is inserted into at least one location in the genome of the mammalian cell.
64. The implantable element of any one of the preceding embodiments, comprising at least one cell-containing compartment which comprises the engineered mammalian cell of any one of the preceding embodiments.
65. The implantable element of any one of the preceding embodiments, further comprising at least one means for mitigating the foreign body response (FBR) when the implantable element is implanted into the subject.
66. The implantable element of any one of embodiments 64-65, wherein the engineered mammalian cell(s) in the at least one cell-containing compartment is derived from an ARPE-19 cell and encapsulated by a polymer composition.
67. The implantable element of embodiment 66, wherein the polymer composition comprises a polymer selected from alginate, hyaluronate, and chitosan.
68. The implantable element of any one of embodiments 66-67, wherein the polymer composition comprises alginate.
69. The implantable element of any one of embodiments 66-67, wherein the polymer composition comprises hyaluronate.
70. The implantable element of any one of embodiments 66-67, wherein the polymer composition comprises chitosan.
71. The implantable element of embodiment 68, wherein the alginate is a high guluronic acid (G) alginate or a high mannuronic acid (M) alginate.
72. The implantable element of embodiment 68, wherein the alginate is a high G alginate.
73. The implantable element of embodiment 68, wherein the alginate is a high M alginate.
74. The implantable element of any one of embodiments 66-73, wherein the polymer composition comprises at least one polymer covalently modified with a peptide.
75. The implantable element of embodiment 74, wherein the peptide comprises, consists essentially of, or consists of GRGDSP, GGRGDSP, or GGGRGDSP.
76. The implantable element of embodiment 75, wherein the peptide comprises, consists essentially of, or consists of GRGDSP.
77. The implantable element of embodiment 75, wherein the peptide comprises, consists essentially of, or consists of GGRGDSP.
78. The implantable element of embodiment 75, wherein the peptide comprises, consists essentially of, or consists of GGGRGDSP.
79. The implantable element of any one of embodiments 66-78, wherein the cell-containing compartment is surrounded by a barrier compartment comprising an alginate hydrogel and optionally a compound of Formula (I) (e.g., a compound of Formula (I) described herein) disposed on the outer surface of the barrier compartment.
80. The implantable element of any one of embodiments 66-79, wherein the polymer composition comprises an alginate covalently modified with a peptide, wherein the peptide consists essentially of or consists of GRGDSP or GGRGDSP, and wherein the barrier compartment comprises an alginate chemically modified with

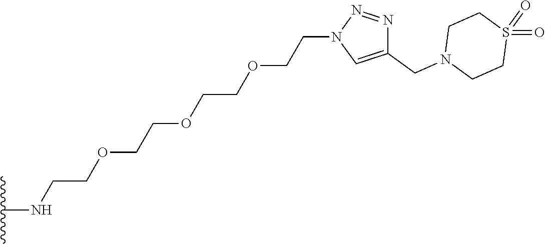 [SMILES: *NCCOCCOCCOCCn1cc(CN2CCS(=O)(=O)CC2)nn1]

or a pharmaceutically acceptable salt thereof.
81. The implantable element of embodiment 80, wherein the polymer composition comprises an alginate covalently modified with a peptide, wherein the peptide consists essentially of or consists of GRGDSP, and wherein the barrier compartment comprises an alginate chemically modified with

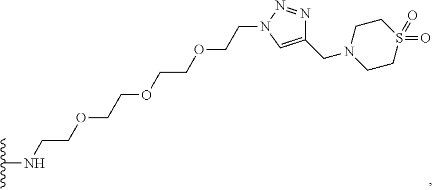 [SMILES: *NCCOCCOCCOCCn1cc(CN2CCS(=O)(=O)CC2)nn1]

or a pharmaceutically acceptable salt thereof.
82. The implantable element of embodiment 80, wherein the polymer composition comprises an alginate covalently modified with a peptide, wherein the peptide consists essentially of or consists of GGRGDSP, and wherein the barrier compartment comprises an alginate chemically modified with

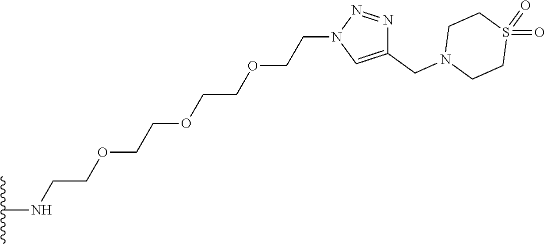 [SMILES: *NCCOCCOCCOCCn1cc(CN2CCS(=O)(=O)CC2)nn1]

or a pharmaceutically acceptable salt thereof.
83. The implantable element of any one of the preceding embodiments, wherein the implantable element is spherical.
84. The implantable element of any one of the preceding embodiments, wherein the implantable element comprises a two-compartment hydrogel capsule.
85. The implantable element of any one of the preceding embodiments, wherein the implantable element is spherical with a diameter of about 0.75 mm to about 2 mm.
86. The implantable element of any one of the preceding embodiments, wherein the therapeutic agent is a protein, e.g., a hormone, a blood clotting factor, an antibody, or an enzyme.
87. The implantable element of any one of the preceding embodiments, wherein the therapeutic agent is a protein.
88. The implantable element of any one of the preceding embodiments, wherein the therapeutic agent is a hormone.
89. The implantable element of any one of the preceding embodiments, wherein the therapeutic agent is a blood clotting factor.
90. The implantable element of any one of the preceding embodiments, wherein the therapeutic agent is an antibody.
91. The implantable element of any one of the preceding embodiments, wherein the therapeutic agent is an enzyme.
92. The implantable element of any one of the preceding embodiments, comprising:
   (i) an engineered ARPE cell capable of reducing the expression of beta-2M; and
   (ii) a polymer composition comprising an alginate covalently modified with one or more of:
   (a) a compound of Formula (I) (e.g., as described herein); and
   (b) a peptide.
93. The implantable element of any one of the preceding embodiments, comprising:
   (i) an engineered ARPE cell capable of reducing the expression of beta-2M; and
   (ii) a polymer composition comprising an alginate covalently modified with a compound of Formula (I) (e.g., as described herein).
94. The implantable element of any one of the preceding embodiments, comprising:
   (i) an engineered ARPE cell capable of reducing the expression of beta-2M; and
   (ii) a polymer composition comprising an alginate covalently modified with a peptide.
95. The implantable element of embodiment 92, wherein the engineered ARPE cell is further capable of reducing the expression of CIITA.
96. The implantable element of any one of the preceding embodiments, comprising:
   (i) an engineered ARPE cell capable of reducing the expression of beta-2M;
   (ii) a polymer composition comprising an alginate covalently modified with one or more of:
   (a)

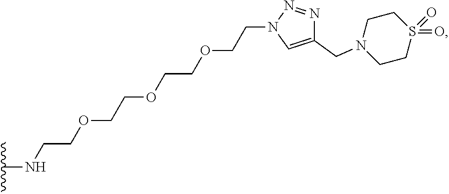 [SMILES: *NCCOCCOCCOCCn1cc(CN2CCS(=O)(=O)CC2)nn1]

pr a pharmaceutically acceptable salt thereof, and
(b) a peptide comprising or consisting of GRGDSP or GGRGDSP.
97. The implantable element of any one of the preceding embodiments, comprising:
   (i) an engineered ARPE cell capable of reducing the expression of beta-2M;
   (ii) a polymer composition comprising an alginate covalently modified with one or more of:
   (a)

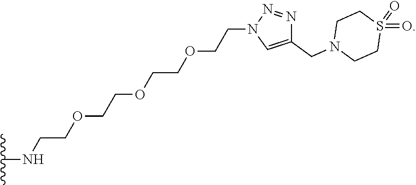 [SMILES: *NCCOCCOCCOCCn1cc(CN2CCS(=O)(=O)CC2)nn1]

or a pharmaceutically acceptable salt thereof, and
(b) a peptide comprising or consisting of GRGDSP.
98. The implantable element of any one of the preceding embodiments, comprising:
   (i) an engineered ARPE cell capable of reducing the expression of beta-2M;
   (ii) a polymer composition comprising an alginate covalently modified with one or more of:
   (a)

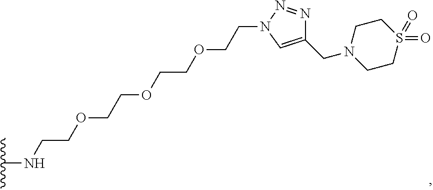 [SMILES: *NCCOCCOCCOCCn1cc(CN2CCS(=O)(=O)CC2)nn1]

or a pharmaceutically acceptable salt thereof, and
(b) a peptide comprising or consisting of GGRGDSP.
99. The implantable element of embodiment 96, wherein the engineered ARPE cell is further capable of reducing the expression of CIITA.
100. The implantable element of any one of the preceding embodiments, formulated for implantation into a subject (e.g., into the intraperitoneal (IP) space, the peritoneal cavity, the omentum, the lesser sac, the subcutaneous fat).
101. The implantable element of any one of the preceding embodiments, formulated for implantation into a subject (e.g., into the intraperitoneal (IP) space).
102. The implantable element of any one of the preceding embodiments, formulated for implantation into a subject (e.g., into the peritoneal cavity).
103. The implantable element of any one of the preceding embodiments, formulated for implantation into a subject (e.g., into the omentum).
104. The implantable element of any one of the preceding embodiments, formulated for implantation into a subject (e.g., into the lesser sac).
105. The implantable element of any one of the preceding embodiments, formulated for implantation into a subject (e.g., into the subcutaneous fat).
106. The implantable element of any one of the preceding embodiments, formulated for implantation into the IP space of a subject.
107. A preparation of implantable elements, wherein each implantable element in the preparation is an implantable element of any one of embodiments 1-106.
108. A method of treating a disease or disorder in a subject, the method comprising administering to the subject an implantable element of any one of embodiments 1-106 or the preparation of embodiment 107, thereby treating the disease or disorder in the subject.
109. The method of embodiment 108, wherein the disease or disorder is a lysosomal storage disease or a metabolic disorder.
110. The method of embodiment 109, wherein the disease or disorder is a lysosomal storage disease.
111. The method of embodiment 109, wherein the disease or disorder is a metabolic disorder.
112. The method any one of embodiments 108-111, wherein the subject is a human.

### EXAMPLES

In order that the disclosure described herein may be more fully understood, the following examples are set forth. The examples described in this application are offered to illustrate the engineered cells, implantable devices, and compositions and methods provided herein and are not to be construed in any way as limiting their scope.

### Example 1: Culturing Exemplary Engineered ARPE-19 Cells for Encapsulation

Exemplary engineered ARPE-19 cells comprising one or more genetic modifications as a stably integrated exogenous transcription unit as described herein may be cultured to produce a composition of cells suitable for encapsulation in two compartment hydrogel capsules. Cells are grown in complete growth medium (DMEM:F12 with 10% FBS) in 150 cm² cell culture flasks or CellSTACK® Culture Chambers (Corning Inc., Corning, NY). To passage cells, the medium in the culture flask is aspirated, and the cell layer is briefly rinsed with phosphate buffered saline (pH 7.4, 137 mM NaCl, 2.7 mM KCl, 8 mM Na₂HPO₄, and 2 mM KH₂PO₄, Gibco). 5-10 mL of 0.05% (w/v) trypsin/0.53 mM EDTA solution (“TrypsinEDTA”) is added to the flask, and the cells are observed under an inverted microscope until the cell layer is dispersed, usually between 3-5 minutes. To avoid clumping, cells are handled with care and hitting or shaking the flask during the dispersion period is minimized. If the cells do not detach, the flasks are placed at 37 °C to facilitate dispersal. Once the cells disperse, 10 mL complete growth medium is added and the cells are aspirated by gentle pipetting. The cell suspension is transferred to a centrifuge tube and spun down at approximately 125×g for 5-10 minutes to remove TrypsinEDTA. The supernatant is discarded, and the cells are resuspended in fresh growth medium. Appropriate aliquots of cell suspension are added to new culture vessels, which are incubated at 37 °C. The medium is renewed weekly.

### Example 2. Reduced Beta-2M Protein Expression by a Beta-2M-Targeting shRNA in IDUA-Expressing ARPE-19 Cells

ARPE-19 cells that had been genetically modified to stably express human IDUA were transduced with shRNA-containing lentiviral particles at a MOI of 100. The shRNA contained either a beta-2M targeting sequence or a scrambled sequence as a negative control. The beta-2M shRNA sequence consisted of AAGTGGAGCATTCAGACTTGTCTTTCAGC.

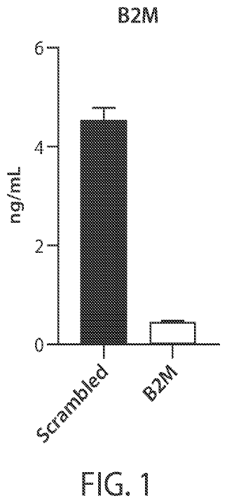

Following transductions, the cells underwent selection with puromycin (1 μg/mL). After selection, the modified cell lines were expanded and characterized using protein specific ELISAs and the results are shown in FIG. 1. Beta-2M protein expression in the IDUA-expressing ARPE-19 cells containing the beta-2M shRNA was 89% lower than in the IDUA-expressing ARPE-19 cells containing the scrambled control shRNA.

### Example 3. Reduced Beta-2M Protein Expression by CRISPR and a Beta-2M-Targeting Guide RNA in ARPE-19 Cells

Unmodified ARPE-19 cells were transduced with lentiviral particles containing a beta-2M-targeting gRNA or a control gRNA with a scrambled sequence and a mCherry fluorescent marker. Following selection with hygromycin, the cells were expanded, and underwent a further transfection with a Cas9/GFP expressing plasmid. Cas9-GFP expressing cells were sorted using flow cytometry to remove the untransfected population. The resulting cells were expanded and characterized by protein-specific ELISA. As shown in FIG. 2, beta-2M expression levels were decreased 99% in the ARPE-19 cells modified with the beta-2M-targeting gRNA compared to ARPE-19 cells modified using the scrambled gRNA.

### Example 4. Reduction of Beta-2M Protein Expression Results in Decreased HLA Class I Expression

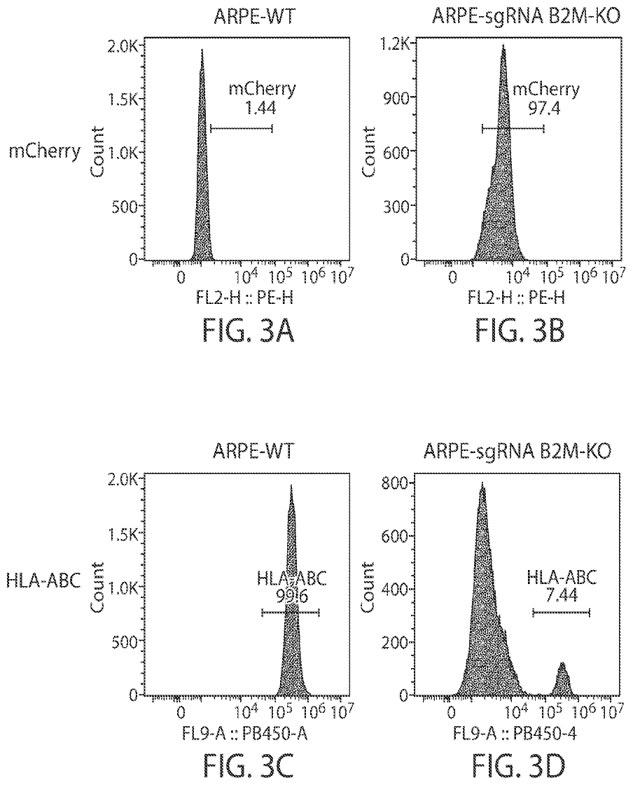

Wild-type ARPE-19 cells and the ARPE19 cells with reduced beta-2M protein expression from previous examples were characterized using flow cytometry. Cells were stained with a fluorescently labelled antibody that recognizes HLA Class 1 ABC. mCherry fluorescence (representative of beta-2M gRNA) and HLA-ABC antibody fluorescence were recorded. 97% of the cells transduced with the B2M-targeting gRNA expressed mCherry (FIG. 3B) compared to 1.4% of the wild type (WT) ARPE-19 cells (FIG. 3A). 99% of the WT cells were stained with the HLA-ABC antibody (FIG. 3C) compared to 7.4% of the cells targeted with beta-2M-targeting gRNA (FIG. 3D). These data confirm targeting beta-2M leads to a corresponding decrease in HLA Class 1 expression on the cell surface.

### Example 5: Preparation of Exemplary Modified Polymers

Chemically-modified Polymer. A polymeric material may be chemically modified with a compound of Formula (I) (or pharmaceutically acceptable salt thereof) prior to formation of a device described herein (e.g., a hydrogel capsule). For example, in the case of alginate, the alginate carboxylic acid is activated for coupling to one or more amine-functionalized compounds to achieve an alginate modified with an afibrotic compound, e.g., a compound of Formula (I). The alginate polymer is dissolved in water (30 mL/gram polymer) and treated with 2-chloro-4,6-dimethoxy-1,3,5-triazine (0.5 eq) and N-methylmorpholine (1 eq). To this mixture is added a solution of the compound of interest (e.g., Compound 101 shown in Table 4) in acetonitrile (0.3M).

The amounts of the compound and coupling reagent added depends on the desired concentration of the compound bound to the alginate, e.g., conjugation density. A medium conjugation density of Compound 101 typically ranges from 2% to 5% N, while a high conjugation density of Compound 101 typically ranges from 5.1% to 8% N. To prepare a solution of low molecular weight alginate, chemically modified with a medium conjugation density of Compound 101 (CM-LMW-Alg-101-Medium polymer), the dissolved unmodified low molecular weight alginate (approximate MW<75 kDa, G:M ratio≥1.5) is treated with 2-chloro-4,6-dimethoxy-1,3,5-triazine (5.1 mmol/g alginate) and N-methylmorpholine (10.2 mmol/g alginate) and Compound 101 (5.4 mmol/g alginate). To prepare a solution of low molecular weight alginate, chemically modified with a high conjugation density of Compound 101 (CM-LMW-Alg-101-High polymer), the dissolved unmodified low-molecular weight alginate (approximate MW<75 kDa, G:M ratio≥1.5) is treated with 2-chloro-4,6-dimethoxy-1,3,5-triazine (5.1 mmol/g alginate) and N-methylmorpholine (10.2 mmol/g alginate) and Compound 101 (10.5 mmol/g alginate).

The reaction is warmed to 55 °C for 16 h, then cooled to room temperature and gently concentrated via rotary evaporation, then the residue is dissolved in water. The mixture is filtered through a bed of cyano-modified silica gel (Silicycle) and the filter cake is washed with water. The resulting solution is then extensively dialyzed (10,000 MWCO membrane) and the alginate solution is concentrated via lyophilization to provide the desired chemically-modified alginate as a solid or is concentrated using any technique suitable to produce a chemically modified alginate solution with a viscosity of 25 cP to 35 cP.

The conjugation density of a chemically modified alginate is measured by combustion analysis for percent nitrogen. The sample is prepared by dialyzing a solution of the chemically modified alginate against water (10,000 MWCO membrane) for 24 hours, replacing the water twice followed by lyophilization to a constant weight.

CBP-Alginates. A polymeric material may be covalenty modified with a cell-binding peptide prior to formation of a device described herein (e.g., a hydrogel capsule described herein) using methods known in the art, see, e.g., Jeon 0, et al., Tissue Eng Part A. 16:2915-2925 (2010) and Rowley, J. A. et al., Biomaterials 20:45-53 (1999).

For example, in the case of alginate, an alginate solution (1%, w/v) is prepared with 50 mM of 2-(N-morpholino)-ethanesulfonic acid hydrate buffer solution containing 0.5M NaCl at pH 6.5, and sequentially mixed with N-hydroxysuccinimide and 1-ethyl-3-[3-(dimethylamino)propyl]carbodiimide (EDC). The molar ratio of N-hydroxysuccinimide to EDC is 0.5:1.0. The peptide of interest is added to the alginate solution. The amounts of peptide and coupling reagent added depends on the desired concentration of the peptide bound to the alginate, e.g., peptide conjugation density. By increasing the amount of peptide and coupling reagent, higher conjugation density can be obtained. After reacting for 24 h, the reaction is purified by dialysis against ultrapure deionized water (diH2O) (MWCO 3500) for 3 days, treated with activated charcoal for 30 min, filtered (0.22 mm filter), and concentrated to the desired viscosity.

The conjugation density of a peptide-modified alginate is measured by combustion analysis for percent nitrogen. The sample is prepared by dialyzing a solution of the chemically modified alginate against water (10,000 MWCO membrane) for 24 hours, replacing the water twice followed by lyophilization to a constant weight.

### Example 5: Preparation of Exemplary Alginate Solutions for Making Hydrogel Capsules

70:30 mixture of chemically-modified and unmodified ainate. A low molecular weight alginate (PRONOVA™ VLVG alginate, NovaMatrix, Sandvika, Norway, cat. #4200506, approximate molecular weight <75 kDa; G:M ratio ≥15) is chemically modified with Compound 101 to produce chemically modified low molecular weight alginate (CM-LMW-Alg-101) solution with a viscosity of 25 cp to 35 cP and a conjugation density of 5.1% to 8% N, as determined by combustion analysis for percent nitrogen. A solution of high molecular weight unmodified alginate (U-HMW-Alg) is prepared by dissolving unmodified alginate (PRONOVA™ SLG100, NovaMatrix, Sandvika, Norway, cat. #4202106, approximate molecular weight of 150 kDa-250 kDa) at 3% weight to volume in 0.9% saline. The CM-LMW-Alg solution is blended with the U-HMW-Alg solution at a volume ratio of 70% CM-LMW-Alg to 30% U-HMW-Alg (referred to herein as a 70:30 CM-Alg:UM-Alg solution).

Unmodified alginate solution. An unmodified medium molecular weight alginate (SLG20, NovaMatrix, Sandvika, Norway, cat. #4202006, approximate molecular weight of 75-150 kDa), is dissolved at 1.4% weight to volume in 0.9% saline to prepare a U-MMW-Alg solution.

Unmodified alginate solution. An unmodified medium molecular weight alginate (SLG20, NovaMatrix, Sandvika, Norway, cat. #4202006, approximate molecular weight of 75-150 kDa), is dissolved at 1.4% weight to volume in 0.9% saline to prepare a U-MMW-Alg solution.

Alginate Solution Comprising Cell Binding Sites. A solution of SLG20 alginate is modified with a peptide consisting of GRGDSP as described above and concentrated to a viscosity of about 100 cP. The amount of the peptide and coupling reagent used are selected to achieve a target peptide conjugation density of about 0.2 to 0.3, as measured by combustion analysis.

### Example 6: Formation of Exemplary Two-Compartment Hydrogel Capsules

Suspensions of engineered mammalian cells as single cells are encapsulated in two-compartment hydrogel capsules according to the protocols described below.

Immediately before encapsulation, a desired volume of a composition comprising the cells (e.g., from a culture of the cells as described in Example 1) are centrifuged at 1,400 r.p.m. for 1 min and washed with calcium-free Krebs-Henseleit (KH) Buffer (4.7 mM KCl, 25 mM HEPES, 1.2 mM KH₂PO₄, 1.2 mM MgSO₄×7H₂O, 135 mM NaCl, pH≈7.4, ≈290 mOsm). After washing, the cells are centrifuged again and all of the supernatant is aspirated. The cell pellet is resuspended in the GRGDSP-modified alginate solution described in Example 3 at a desired cell density (e.g., about 50 to 150 million suspended single cells per ml alginate solution).

Prior to fabricating hydrogel capsules, buffers and alginate solutions are sterilized by filtration through a 0.2-μm filter using aseptic processes.

To prepare two-compartment hydrogel millicapsules of about 1.5 mm diameter, an electrostatic droplet generator is set up as follows: an ES series 0-100-kV, 20-watt high-voltage power generator (EQ series, Matsusada, NC, USA) is connected to the top and bottom of a coaxial needle (inner lumen of 22G, outer lumen of 18G, Ramd-Hart Instrument Co., Succasunna, NJ, USA). The inner lumen is attached to a first 5-ml Luer-lock syringe (BD, NJ, USA), which is connected to a syringe pump (Pump 11 Pico Plus, Harvard Apparatus, Holliston, MA, USA) that is oriented vertically. The outer lumen is connected via a luer coupling to a second 5-ml Luer-lock syringe which is connected to a second syringe pump (Pump 11 Pico Plus) that is oriented horizontally. A first alginate solution containing the genetically modified cells (as single cells) suspended in a GRGDSP-modified alginate solution is placed in the first syringe and a cell-free alginate solution comprising a mixture of a chemically-modified alginate and unmodified alginate is placed in the second syringe. The two syringe pumps move the first and second alginate solutions from the syringes through both lumens of the coaxial needle and single droplets containing both alginate solutions are extruded from the needle into a glass dish containing a cross-linking solution. The settings of each Pico Plus syringe pump are 12.06 mm diameter and the flow rates of each pump are adjusted to achieve a flow rate ratio of 1:1 for the two alginate solutions. Thus, with the total flow rate set at 10 ml/h, the flow rate for each alginate solution is about 5 mL/h. Control (empty) capsules are prepared in the same manner except that the alginate solution used for the inner compartment is a cell-free solution.

After extrusion of the desired volumes of alginate solutions, the alginate droplets are crosslinked for five minutes in a cross-linking solution which contain 25 mM HEPES buffer, 20 mM BaCl₂, 0.2M mannitol and 0.01% of poloxamer 188. Capsules that fall to the bottom of the crosslinking vessel are collected by pipetting into a conical tube. After the capsules settle in the tube, the crosslinking buffer is removed, and capsules are washed four times in HEPES buffer, two times in 0.9% saline, and two times in culture media and stored in an incubator at 37 °C.

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present disclosure, as defined in the following claims.

1. An implantable element comprising an engineered mammalian cell, wherein:
(i) the engineered mammalian cell comprises a reduction in the level or function of a major histocompatibility complex (MHC) class I protein complex; and
(ii) the engineered mammalian cell comprises an exogenous nucleic acid encoding a therapeutic agent;
wherein the implantable element comprises a polymer and a compound of Formula (I): 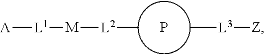
or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₁-C₆-alkenylene)-, —N(R^{C})N(R^{D})—, —NCN—, —C(═N(R^{C})(R^{D}))O—, —S—, —S(O)ₓ, —OS(O)ₓ, —N(R^{C})S(O)ₓ, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is absent, cycloalkyl, heterocyclyl, or heteroaryl, each of which is optionally substituted by one or more R⁴;
Z is hydrogen, alkyl, alkenyl, alkynyl, heteroalkyl, —OR^{A}, —C(O)R^{A}, —C(O)OR^{A}, —C(O)N(R^{C})(R^{D}), N(R^{C})CFv^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

2. The implantable element of claim 1, wherein the MHC class I protein complex comprises one or more of:
(i) human leukocyte antigen (HLA) A;
(ii) HLA-B;
(iii) HLA-C; and
(iv) beta-2-microglobulin (beta-2M).

3. The implantable element of claim 2, comprising (i).

4. The implantable element of any one of claims 2-3, comprising (ii).

5. The implantable element of any one of claims 2-4, comprising (iii).

6. The implantable element of any one of claims 2-5, comprising (iv).

7. The implantable element of any one of the preceding claims, wherein the engineered mammalian cell comprises a mutation resulting in the reduction of expression of a component of the MHC class I complex, e.g., as compared to a reference standard.

8. The implantable element of any one of the preceding claims, wherein the engineered mammalian cell comprises a lower-functioning or non-functioning variant of a component of the MHC class I component, e.g., as compared to a reference standard.

9. The implantable element of any one of the preceding claims, wherein expression of a component of the MHC class I complex is silenced or knocked down, e.g., as compared to a reference standard.

10. The implantable element of any one of the preceding claims, wherein the engineered mammalian cell comprises a reduction in the level of a MHC class I component by about 0.05%, 0.1%, 0.5%, 0.75%, %, 21%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the level of an MHC class I component, e.g., as compared to a reference standard.

11. The implantable element of any one of the preceding claims, wherein the engineered mammalian cell comprises a reduction in the level of a MHC class I component between 1-25%, 5-25%, 10-25%, 25-50%, 25-75%, or 50-75%, e.g., as compared to an engineered mammalian cell that is substantially identical to or identical to the engineered mammalian cell except that it does not comprise a reduction in the level of an MHC class I component, e.g., as compared to a reference standard.

12. The implantable element of any one of the preceding claims, wherein the engineered mammalian cell comprises a reduction in the level of a MHC class I component of greater than 50%, greater than 75% or greater than 90%, e.g., as compared to an engineered mammalian cell that is substantially identical to or identical to the engineered mammalian cell except that it does not comprise a reduction in the level of an MHC class I component, e.g., as compared to a reference standard.

13. The implantable element of any one of the preceding claims, wherein the engineered mammalian cell comprises a reduction in the function of a MHC class I component by about 0.05%, 0.1%, 0.5%, 0.75%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, e.g., as compared to an engineered mammalian cell that is substantially identical to or identical to the engineered mammalian cell except that it does not comprise a reduction in the function of an MHC class I component, e.g., as compared to a reference standard.

14. The implantable element of any one of the preceding claims, wherein the engineered mammalian cell comprises a reduction in the function of a MHC class I component between 1-25%, 5-25%, 10-25%, 25-50%, 25-75%, 50-75%, or 75-100%, e.g., as compared to an engineered mammalian cell that is substantially identical to or identical to the engineered mammalian cell except that it does not comprise a reduction in the function of an MHC class I component, e.g., as compared to a reference standard.

15. The implantable element of any one of the preceding claims, wherein the engineered mammalian cell comprises a reduction in the function of a MHC class I component of greater than 50%, greater than 75% or greater than 90%, e.g., as compared to an engineered mammalian cell that is substantially identical to or identical to the engineered mammalian cell except that it does not comprise a reduction in the function of an MHC class I component, e.g., as compared to a reference standard.

16. The implantable element of any one of the preceding claims, wherein the engineered mammalian cell further comprises a reduction in the level or function of a MHC class II complex.

17. The implantable element of claim 16, wherein the MHC class II complex comprises one or more of:
(v) human leukocyte antigen (HLA) DP;
(vi) HLA-DM;
(vii) HLA-DOA;
(viii) HLA-DOB;
(ix) HLA-DQ; and
(x) HLA-DR.

18. The implantable element of claim 17, wherein the MHC class II complex comprises (v).

19. The implantable element of any one of claims 17-18, wherein the MHC class II complex comprises (vi).

20. The implantable element of any one of claims 17-19, wherein the MHC class II complex comprises (vii).

21. The implantable element of any one of claims 17-20, wherein the MHC class II complex comprises (viii).

22. The implantable element of any one of claims 17-21, wherein the MHC class II complex comprises (ix).

23. The implantable element of any one of claims 17-22, wherein the MHC class II complex comprises (x).

24. The implantable element of any one of claims 17-23, wherein the cell comprises a reduction in the function or expression of a class II major histocompatibility complex transactivator (CIITA).

25. The implantable element of any one of the preceding claims, wherein the engineered mammalian cell is a human cell.

26. The implantable element of any one of the preceding claims, wherein the engineered mammalian cell comprises an embryonic stem cell (ESC) or an induced pluripotent stem cell (iPSC).

27. The implantable element of any one of the preceding claims, wherein the engineered mammalian cell comprises a retinal pigment epithelial (RPE) cell, a CCD-33Lu cell, a MRC-5 cell, a MRC-9 cell, a MCF10a cell, or a cell derived therefrom.

28. The implantable element of any one of the preceding claims, wherein the engineered mammalian cell comprises an engineered retinal pigment epithelial (RPE) cell (e.g., an engineered ARPE-19 cell).

29. The implantable element of any one of the preceding claims, wherein the exogenous nucleotide sequence is extrachromosomal.

30. The implantable element of any one of the preceding claims, wherein the exogenous nucleotide sequence is inserted into at least one location in the genome of the mammalian cell.

31. The implantable element of any one of the preceding claims, comprising at least one cell-containing compartment which comprises the engineered mammalian cell of any one of claims 1-30.

32. The implantable element of any one of the preceding claims, wherein the polymer is selected from alginate, hyaluronate, and chitosan.

33. The implantable element of claim 32, wherein the polymer comprises alginate.

34. The implantable element of any one of claims 32-33, wherein the alginate is a high guluronic acid (G) alginate or a high mannuronic acid (M) alginate.

35. The implantable element of any one of the preceding claims, wherein the polymer comprises at least one polymer covalently modified with a peptide.

36. The implantable element of claim 35, wherein the peptide comprises, consists essentially of or consists of GRGDSP, GGRGDSP or GGGRGDSP.

37. The implantable element of any one of claims 31-36, wherein the cell-containing compartment is surrounded by a barrier compartment comprising an alginate hydrogel and optionally a compound of Formula (I) (e.g., a compound of Formula (I) described herein) disposed on the outer surface of the barrier compartment.

38. The implantable element of any one of claims 31-37, wherein the polymer comprises an alginate covalently modified with a peptide, wherein the peptide consists essentially of or consists of GRGDSP or GGRGDSP, and wherein the barrier compartment comprises an alginate chemically modified with 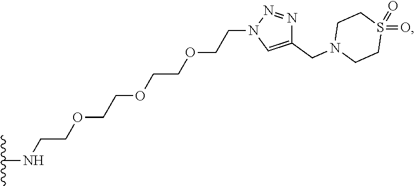 [SMILES: *NCCOCCOCCOCCn1cc(CN2CCS(=O)(=O)CC2)nn1]
or a pharmaceutically acceptable salt thereof.

39. The implantable element of any one of the preceding claims, wherein the implantable element is spherical.

40. The implantable element of any one of the preceding claims, wherein the implantable element comprises a two-compartment hydrogel capsule.

41. The implantable element of any one of the preceding claims, wherein the implantable element is spherical with a diameter of about 0.75 mm to about 2 mm.

42. The implantable element of any one of the preceding claims, wherein the therapeutic agent is a protein, e.g., a hormone, a blood clotting factor, an antibody, or an enzyme.

43. The implantable element of any one of the preceding claims, comprising:
(i) an engineered ARPE cell capable of reducing the expression of beta-2M;
(ii) a polymer composition comprising an alginate covalently modified with one or more of:
(a) a compound of Formula (I) (e.g., as described herein); and
(b) a peptide.

44. The implantable element of claim 43, wherein the engineered ARPE cell is further capable of reducing the expression of CIITA.

45. The implantable element of any one of the preceding claims, comprising:
(i) an engineered ARPE cell capable of reducing the expression of beta-2M;
(ii) a polymer composition comprising an alginate covalently modified with one or more of:
(a) 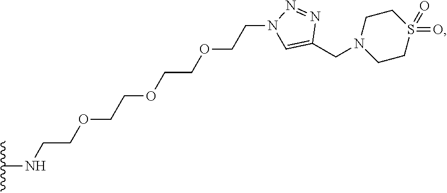 [SMILES: *NCCOCCOCCOCCn1cc(CN2CCS(=O)(=O)CC2)nn1]
or a pharmaceutically acceptable salt thereof, and
(b) a peptide comprising or consisting of GRGDSP or GGRGDSP.

46. The implantable element of claim 47, wherein the engineered ARPE cell is further capable of reducing the expression of CIITA.

47. The implantable element of any one of the preceding claims, formulated for implantation into a subject (e.g., into the intraperitoneal (IP) space, the peritoneal cavity, the omentum, the lesser sac, the subcutaneous fat).

48. The implantable element of any one of the preceding claims, formulated for implantation into the IP space of a subject.

49. A preparation of implantable elements, wherein each implantable element in the preparation is an implantable element of any one of claims 1-48.

50. A composition for use in treating a disease or disorder in a subject comprising an implantable element of any one of claims 1-48 or the preparation of claim 49.

51. The composition for use of claim 50, wherein the disease or disorder is a lysosomal storage disease or a metabolic disorder.

52. The composition for use of any one of claims 50-51, wherein the subject is a human.
